(19) United States
(12) Patent Application Publication
(10) Pub. No.: US 2013/0266566 A1
(43) Pub. Date: Oct. 10, 2013

(54) METHODS OF INHIBITING FIBROSIS USING ANTI-PAI-1 ANTIBODIES

(71) Applicants: Ian Anderson, Washington, DC (US); Anthony Coyle, Boston, MA (US); Nancy Noble, Salt Lake City, UT (US)

(72) Inventors: Ian Anderson, Washington, DC (US); Anthony Coyle, Boston, MA (US); Nancy Noble, Salt Lake City, UT (US)

(73) Assignee: Abbott Laboratories, Abbott Park, IL (US)

(21) Appl. No.: 13/848,197

(22) Filed: Mar. 21, 2013

Related U.S. Application Data
(63) Continuation of application No. 13/571,646, filed on Aug. 10, 2012, now abandoned.
Application No. 13/571,646 is a continuation of application No. 13/333,447, filed on Dec. 21, 2011, now abandoned.
Application No. 13/333,447 is a continuation of application No. 13/099,090, filed on May 2, 2011, now abandoned.
(60) Provisional application No. 61/330,692, filed on May 3, 2010.
(60) Provisional application No. 61/330,584, filed on May 3, 2010.

(51) Int. Cl. C07K 16/18

(52) U.S. Cl. CPC C07K 16/18
USPC 424/135.1; 424/133.1; 424/178.1; 530/395; 530/350; 424/172.1; 424/142.1

(57) ABSTRACT

Provided are anti-PAI-1 antibodies or antibody fragments and methods of using them.

### CROSS-REFERENCE TO RELATED APPLICATION

This application is a continuation of U.S. patent application Ser. No. 13/571,646, filed on Aug. 10, 2012, which is a continuation of U.S. patent application Ser. No. 13/333,447, filed on Dec. 21, 2011, and a continuation of U.S. patent application Ser. No. 13/099,090, filed on May 2, 2011. This application also claims priority to U.S. Provisional Application Ser. No. 61/330,692 filed on May 3, 2010; and U.S. Provisional Application Ser. No. 61/330,584 filed on May 3, 2010. The entire contents of each of the foregoing applications are incorporated herein by reference.

### FIELD OF THE DISCLOSURE

The present disclosure relates to antibody molecules which bind plasminogen activator inhibitor 1 (PAI-1). The antibodies are useful for inhibiting fibrosis. The antibodies are useful for treating diseases and conditions caused or exacerbated by fibrosis, such as systemic lupus erythromatosus (SLE), scleroderma, pulmonary fibrosis (wherein the disease is not idiopathic pulmonary fibrosis (IPF)), diabetic nephropathy, lupus nephritis, graft versus host disease, glomerulonephritis, focal segmental glomerulosclerosis, membranous nephropathy, mesangial proliferative glomerulonephritis, membranoproliferative glomerulonephritis, renal fibrosis, and COPD.

### BACKGROUND OF THE DISCLOSURE

The fibrinolytic system plays important roles in physiologic events such as thrombolysis. The fibrinolytic system is activated when plasminogen activator (PA) converts plasminogen to plasmin. Tissue plasminogen activator converts plasminogen (i.e., the precursor of plasmin) to plasmin. Plasmin converts fibrin to a fibrin degradation product by breaking it down. Plasminogen activator inhibitor-1 (PAI-1) is a major regulatory component of the plasminogen-plasmin system. PAI-1 is a serine protease inhibitor, and is the principal physiologic inhibitor of both tissue type plasminogen activator (t-PA) and urokinase type plasminogen activator (u-PA).

### SUMMARY OF THE DISCLOSURE

There is a need for improved therapies for treating a variety of conditions caused or exacerbated, in whole or in part, by fibrosis and/or inflammation. The present disclosure provides methods for treating such diseases and conditions using anti-PAI-1 antagonistic agents. Anti-PAI-1 antibodies for use in the claimed methods have favorable properties, such as specificity for human PAI-1, cross-reactivity with species used as disease models, affinity, ability to inhibit PAI-1 function without inhibiting binding to vitronectin, etc. Moreover, anti-PAI-1 antibodies for use in the claimed methods have functional properties that make them particularly suitable for use in treating fibrotic conditions, such as the ability to stimulate plasmin-mediated activation of MMP-1.

In a first aspect, the disclosure provides a method of treating a disease or condition in a subject in need thereof, such as a subject having a disease or condition selected from one or more of: systemic lupus erythromatosus (SLE), scleroderma, pulmonary fibrosis, diabetic nephropathy, lupus nephritis, graft versus host disease, glomerulonephritis, focal segmental glomerulosclerosis, membranous nephropathy, mesangial proliferative glomerulonephritis, membranoproliferative glomerulonephritis, renal fibrosis, and COPD. In one embodiment, the disease does not include IPF. The method comprises administering to said subject an effective amount of a human or chimeric antibody or antibody fragment, wherein the antibody or antibody fragment immunospecifically binds to human PAI-1 and inhibits PAI-1 activity.

In a related aspect, the disclosure provides a method of treating a disease or condition in a subject in need thereof, such as a subject having a disease or condition selected from one or more of: systemic lupus erythromatosus (SLE), scleroderma, pulmonary fibrosis, wherein pulmonary fibrosis does not include IPF, diabetic nephropathy, lupus nephritis, graft versus host disease, glomerulonephritis, focal segmental glomerulosclerosis, membranous nephropathy, mesangial proliferative glomerulonephritis, membranoproliferative glomerulonephritis, renal fibrosis, and COPD. The method comprises administering to said subject an effective amount of a human or chimeric antibody or antibody fragment, wherein the antibody or antibody fragment immunospecifically binds to human PAI-1, and can stimulate plasmin-mediated activation of MMP-1.

In a related aspect, the disclosure provides a method of inhibiting fibrosis in a patient in need thereof. The method comprises administering an effective amount of any of the antibodies of the disclosure to, for example, inhibit PAI-1 activity and stimulate plasmin-mediated activation of MMP-1 in said patient. Exemplary patients include, for example, patients having systemic lupus erythromatosus (SLE), scleroderma, pulmonary fibrosis, wherein pulmonary fibrosis does not include IPF, diabetic nephropathy, lupus nephritis, graft versus host disease, glomerulonephritis, focal segmental glomerulosclerosis, membranous nephropathy, mesangial proliferative glomerulonephritis, membranoproliferative glomerulonephritis, renal fibrosis, and COPD.

The following features describe certain embodiments of any of the foregoing aspects and embodiments of the disclosure. In other words, the following features describe certain embodiments that may describe any antibodies for use in the claimed methods of the disclosure, as well as other features of the claimed methods. Such embodiments may be present alone or in any combination, and the disclosure contemplates all suitable combinations of aspects and embodiments of the disclosure.

In certain embodiments, the antibody or antibody fragment is administered as a composition comprising a purified antibody or antibody fragment and a pharmaceutically acceptable carrier. In certain embodiments, the composition is a pyrogen-free composition. In certain embodiments, the antibody or antibody fragment is a human antibody or antibody fragment. In certain embodiments, the antibody or antibody fragment is a monoclonal antibody

In certain embodiments, the antibody or antibody fragment has two or more of the following characteristics (“The Desired Characteristics”):

(a) affinity (K_{D}) between 5 pm to 200 pM for active human PAI-1, as assessed by surface plasmon resonance;

(b) immunospecifically binds to a human PAI-1:vitronectin complex, but does not disrupt the binding of human PAI-1 to vitronectin;

(c) immunospecifically binds to glycosylated human PAI-1;

(d) does not immunospecifically bind to human PAI-2 or human PAI-3;

(e) inhibits the binding of human PAI-1 to tPA by at least 50%;

(f) inhibits the binding of human PAI-1 to tPA with an IC₅₀ of about 5 nM or less;

(g) inhibits the binding of human PAI-1 to uPA by at least 50%;

(h) immunospecifically binds human PAI-1, mouse PAI-1, and rat PAI-1;

(i) immunospecifically binds human PAI-1, mouse PAI-1, and PAI-1 from at least one non-human primate;

(j) immunospecifically binds cynomolgus PAI-1;

(k) immunospecifically binds to a human PAI-1 epitope chosen from SEQ ID NOs: 156-158;

(l) specifically binds to the reactive center loop of PAI-1 that interacts with tPA and uPA;

(m) preferentially binds to active human PAI-1 over latent human PAI-1;

(n) binds to the same epitope as Antibody 8;

(o) immunospecifically binds to human PAI-1 with an affinity at least one order of magnitude greater than its affinity for mouse and/or rat PAI-1;

(p) can reduce the level of VCAM-1 in dermal tissues;

(q) reduces the level of TNF-alpha in dermal tissues;

(r) stimulates plasmin-mediated activation of MMP-1.

In certain embodiments, the antibody or antibody fragment has at least any number of these characteristics, such as at least 3, 4, 5, 6, 7, 8, 9, 10, 11, 12, 13, 14, 15, 16. Of course, such antibodies may optionally have additional charateristics.

In certain embodiments, the antibody or antibody fragment immunospecifically binds cynomolgus PAI-1 with approximately the same affinity as human PAI-1.

In certain embodiments, the antibody or antibody fragment can reduce the level of VCAM-1 in dermal tissues; can reduce the level of TNF-alpha in dermal tissues; and can stimulate plasmin-mediated activation of MMP-1.

In certain embodiments, the antibody or antibody fragment is a human or chimeric antibody or antibody fragment that immunospecifically binds to human PAI-1 and inhibits PAI-1 activity, and has at least one or more (e.g., at least 1, 2, 3) of the following characteristics:

affinity (K_{D}) between 5 pm to 200 pM for active human PAI-1, as assessed by surface plasmon resonance;

immunospecifically binds to a human PAI-1:vitronectin complex, but does not disrupt the binding of human PAI-1 to vitronectin;

specifically binds to the reactive center loop of PAI-1 that interacts with tPA and uPA.

In certain embodiments, the antibody or antibody fragment has an affinity (K_{D}) of between about 5 pM and 125 pM for active human PAI-1, as assessed by surface plasmon resonance.

In certain embodiments, the antibody or antibody fragment has an affinity (K_{D}) of less than or equal to about 100 pM for active human PAI-1, as assessed by surface plasmon resonance.

In certain embodiments, the antibody or antibody fragment has an affinity (K_{D}) of less than or equal to 50 pM for active human PAI-1, as assessed by surface plasmon resonance.

In certain embodiments, the antibody or antibody fragment is one or more of: a monoclonal antibody; a human antibody; a chimeric antibody; a single-chain Fv (scFv); an Fab fragment; an F(ab′) fragment; an intrabody; and a synthetic antibody.

In certain embodiments, the antibody or antibody fragment is conjugated to a detectable substance or a therapeutic agent.

In certain embodiments, the method comprises administering a composition comprising an anti-PAI-1 antibody, such as a pyrogen-free composition. In certain embodiments, the composition is a sterile composition.

In certain embodiments, the method comprises administering the antibody or antibody fragment (or composition) systemically. In certain embodiments, systemic administration is intravenous or subcutaneous administration.

In certain embodiments, the subject in need thereof is a subject having SLE, and the method comprises treating SLE. In certain embodiments, treating SLE comprises treating lupus nephritis associated with SLE.

In certain embodiments, the subject in need thereof is a subject having scleroderma, and the method comprises treating scleroderma. In certain embodiments, treating comprises treating digital ulcers associated with scleroderma.

In certain embodiments, the subject in need thereof is a subject having lupus nephritis, and the method comprises treating lupus nephritis.

In certain embodiments, the subject in need thereof is a subject having diabetic nephropathy, and the method comprises treating diabetic nephropathy.

In certain embodiments, the subject in need thereof is a subject having COPD.

In certain embodiments, the antibody or antibody fragment is administered as part of a therapeutic regimen along with one or more medicaments or other treatment modalities appropriate for the disease or condition.

In certain embodiments, treatment with the antibody or antibody fragment delays the need for or decreases the frequency of dialysis. In certain embodiments, treatment with the antibody or antibody fragment delays the need for a kidney or lung transplant.

In certain embodiments, the subject is human.

The following embodiments also apply to any of the foregoing aspects and embodiments of the disclosure and point out particular antibodies and antibody fragments suitable, for example, for use in the claimed methods. Such antibodies and antibody fragments are, in certain embodiments, exemplary of anti-PAI-1 antibodies suitable for use in the claimed methods.

In certain embodiments, the antibody or antibody fragment has a particular sequence of the CDRs of the VH and/or VL. By way of example, the antibody or antibody fragment may comprise

a VH CDR1 having an amino acid sequence identical to or comprising 1, 2, or 3 amino acid residue substitutions relative to SEQ ID NO: 113;

a VH CDR2 having an amino acid sequence identical to or comprising 1, 2, or 3 amino acid residue substitutions relative to SEQ ID NO: 114;

a VH CDR3 having an amino acid sequence identical to or comprising 1, 2, or 3 amino acid residue substitutions relative to SEQ ID NO: 115;

a VL CDR1 having an amino acid sequence identical to or comprising 1, 2, or 3 amino acid residue substitutions relative to SEQ ID NO:;

a VL CDR2 having an amino acid sequence identical to or comprising 1, 2, or 3 amino acid residue substitutions relative to SEQ ID NO: 117; and

a VL CDR3 having an amino acid sequence identical to or comprising 1, 2, or 3 amino acid residue substitutions relative to SEQ ID NO: 118.

By way of further example, the antibody or antibody fragment may have a sequence of any CDR1, CDR2, CDR3 of the VH or VL of the antibodies set forth in Table 2, 16, and the Sequence Listing.

In certain embodiments, the antibody or antibody fragments comprise one or more substitutions, insertions, or deletions. In certain embodiments, an amino acid substitution is a conservative substitution. In certain embodiments, an amino acid substitution is a non-conservative substitution.

In certain embodiments, the antibody or antibody fragment for use in the claimed methods immuno specifically binds to human PAI-1, inhibits human PAI-1 activity and comprises:

a VH CDR1 having an amino acid sequence identical to or comprising 1, 2, or 3 amino acid residue substitutions relative to SEQ ID NO: 113 or 187;

a VH CDR2 having an amino acid sequence identical to or comprising 1, 2, or 3 amino acid residue substitutions relative to SEQ ID NO: 161;

a VH CDR3 having an amino acid sequence identical to or comprising 1, 2, or 3 amino acid residue substitutions relative to SEQ ID NO: 171;

a VL CDR1 having an amino acid sequence identical to or comprising 1, 2, or 3 amino acid residue substitutions relative to SEQ ID NO: 176;

a VL CDR2 having an amino acid sequence identical to or comprising 1, 2, or 3 amino acid residue substitutions relative to SEQ ID NO: 117; and

a VL CDR3 having an amino acid sequence identical to or comprising 1, 2, or 3 amino acid residue substitutions relative to SEQ ID NO: 118 or 186.

In certain embodiments, the antibody or antibody fragment comprises one or more amino acid substitutions, wherein said amino acid substitutions are at one or more amino acid residues selected from among the members of the group consisting of:

Kabat residue 31 of the VH CDR1;

Kabat residue 53 of the VH CDR2;

Kabat residue 55 of the VH CDR2;

Kabat residue 60 of the VH CDR2;

Kabat residue 63 of the VH CDR2;

Kabat residue 64 of the VH CDR2;

Kabat residue 65 of the VH CDR2;

Kabat residue 96 of the VH CDR3; and

Kabat residue 100C of the VH CDR3.

In another embodiment, the antibody or antibody fragment comprises one or more amino acid substitutions, wherein said amino acid substitutions are at one or more amino acid residues selected from among the members of the group consisting of:

Kabat residue 24 of the VL CDR1;

Kabat residue 28 of the VL CDR1;

Kabat residue 31 of the VL CDR1;

Kabat residue 32 of the VL CDR1;

Kabat residue 50 of the VL CDR2;

Kabat residue 52 of the VL CDR2; and

Kabat residue 93 of the VL CDR3.

In another embodiment, the antibody or antibody fragment comprises one or more amino acid substitutions, wherein the amino acid substitutions comprise one or more substitutions selected from among the members of the group consisting of:

Gly at Kabat residue 31 of the VH CDR1;

Thr or Ala at Kabat residue 53 of the VH CDR2;

Pro, Gly, Ser, or Val at Kabat residue 55 of the VH CDR2;

Ser at Kabat residue 60 of the VH CDR2;

Leu at Kabat residue 63 of the VH CDR2;

Arg at Kabat residue 64 of the VH CDR2;

Ser at Kabat residue 65 of the VH CDR2;

Arg at Kabat residue 96 of the VH CDR3;

Arg at Kabat residue 100C of the VH CDR3;

Gln at Kabat residue 24 of the VL CDR1;

Ser at Kabat residue 28 of the VL CDR1;

Arg at Kabat residue 31 of the VL CDR1;

Ser, Glu, Gln, Thr, Asn, Phe, or Ala at Kabat residue 32 of the VL CDR1;

Arg at Kabat residue 50 of the VL CDR2;

Thr at Kabat residue 52 of the VL CDR2; and

Asp at Kabat residue 93 of the VL CDR3.

In another embodiment, the VH CDR1 comprises an amino acid sequence selected from SEQ ID NO: 113 or SEQ ID NO: 159.

In another embodiment, the VH CDR2 comprises an amino acid sequence chosen from: SEQ ID NOs: 114 and 160-169.

In another embodiment, the VH CDR3 comprises an amino acid sequence chosen from: SEQ ID NOs: 115 and 170-171.

In another embodiment, the VL CDR1 comprises an amino acid sequence chosen from: SEQ ID NOs: and 172-182.

In another embodiment, the VL CDR2 comprises an amino acid sequence chosen from: SEQ ID NOs: 117 and 183-184.

In another embodiment, the VH CDR3 comprises an amino acid sequence selected from SEQ ID NO: 118 or SEQ ID NO: 185.

The sequence of these CDRs may be present in any combination.

In another embodiment, the human or chimeric antibody or antibody fragment for use in the claimed method immunospecifically binds to human PAI-1, inhibits human PAI-1 activity and comprises:

a VH CDR1 having the amino acid sequence XYAIS, wherein X is a neutral amino acid residue;

a VH CDR2 having the amino acid sequence GIIPX¹FX²TANYX³QKX⁴X⁵X⁶, wherein
X1 is a neutral amino acid residue;
X2 is a neutral or acidic amino acid residue;
X3 is a neutral amino acid residue;
X⁴ is a neutral amino residue;
X⁵ is a neutral or basic amino acid residue;
X⁶ is a neutral amino acid residue; and

a VH CDR3 having the amino acid sequence EX¹RQWLEGX²FDY, wherein
X¹ is a basic amino acid residue;
X² is a basic amino acid residue.

In certain embodiments, the antibody or antibody fragment comprises:

a VH CDR1 having the amino acid sequence XYAIS, wherein X is an amino acid chosen from Ser, Thr, Gly, Ala, Val, Leu, and Ile.

In certain embodiments, the antibody or antibody fragment comprises:

a VH CDR2 having the amino acid sequence GIIPX¹FX²TANYX³QKX⁴X⁵X⁶, wherein
X1 is an amino acid chosen from Ile, Ser, Thr, Gly, Ala, Val, and Leu;
X2 is an amino acid chosen from Asp, Asn, Glu, Gln, Gly, Ala, Val, Leu, Ile, Ser, Thr, and Pro;
X3 is an amino acid chosen from Ala, Ser, Thr, Gly, Val, Leu, and Ile;
X⁴ is an amino acid chosen from Phe, Leu, Ile, Ala, Ser, Thr, Gly, Ala, and Val;
X⁵ is an amino acid chosen from Gln, Asn, Arg, Lys, and His;
X⁶ is an amino acid chosen from Gly, Ser, Thr, Ala, Val, Leu, and Ile.

In certain embodiments, the antibody or antibody fragment comprises:

a VH CDR3 having the amino acid sequence EX¹RQWLEGX²FDY, wherein
X¹ is an amino acid chosen from Lys, Arg, and His;
X² is an amino acid chosen from Lys, Arg, and His.

In certain embodiments, the antibody or antibody fragment comprises:

a VH CDR1 having the amino acid sequence XYAIS, wherein X is an amino acid chosen from Ser, Thr, Gly, Ala, Val, Leu, and Ile;

a VH CDR2 having the amino acid sequence GIIPX¹FX²TANYX³QKX⁴X⁵X⁶, wherein
X1 is an amino acid chosen from Ile, Ser, Thr, Gly, Ala, Val, and Leu;
X2 is an amino acid chosen from Asp, Asn, Glu, Gln, Gly, Ala, Val, Leu, Ile, Ser, Thr, and Pro;
X3 is an amino acid chosen from Ala, Ser, Thr, Gly, Ala, Val, Leu, and Ile;
X⁴ is an amino acid chosen from Phe, Leu, Ile, Ala, Ser, Thr, Gly, Ala, and Val;
X⁵ is an amino acid chosen from Gln, Asn, Arg, Lys, and His;
X⁶ is an amino acid chosen from Gly, Ser, Thr, Ala, Val, Leu, and Ile; and

a VH CDR3 having the amino acid sequence EX¹RQWLEGX²FDY, wherein
X¹ is an amino acid chosen from Lys, Arg, and His;
X² is an amino acid chosen from Lys, Arg, and His.

In certain embodiments, the antibody or antibody fragment comprises:

a VL CDR1 having the amino acid sequence X¹ASEX²IYX³X⁴LA, wherein
X¹ is an amino acid chosen from Arg, Lys, His, Gln, and Asn;
X² is an amino acid chosen from Gly, Ser, Thr, Ala, Val, Leu, and Ile;
X³ is an amino acid chosen from Lys, Arg, and His;
X⁴ is an amino acid chosen from Asp, Glu, Asn, and Gln;

a VL CDR2 having the amino acid sequence X¹AX²SLAS, wherein
X¹ is an amino acid chosen from Lys, Arg, and His;
X² is an amino acid chosen from Gly, Ser, Thr, Ala, Val, Leu, and Ile; and

a VL CDR3 having the amino acid sequence QQYSXYPLT, wherein X is an amino acid chosen from Asn, Asp, Glu, and Gln.

In another embodiment, the human or chimeric antibody or antibody fragment for use in the claimed immuno specifically binds to human PAI-1, inhibits human PAI-1 activity and comprises:

a VL CDR1 having the amino acid sequence X¹ASEX²IYX³X⁴LA, wherein
X¹ is a basic or neutral amino acid residue;
X² is a neutral amino acid residue;
X³ is a basic amino acid residue;
X⁴ is an acidic or neutral amino acid residue;

a VL CDR2 having the amino acid sequence X¹AX²SLAS, wherein
X¹ is a basic amino acid residue;
X² is a neutral amino acid residue; and

a VL CDR3 having the amino acid sequence QQYSXYPLT, wherein X is an acidic or neutral amino acid residue.

In certain embodiments, the antibody or antibody fragment comprises:

a VL CDR1 having the amino acid sequence X¹ASEX²IYX³X⁴LA, wherein
X¹ is an amino acid chosen from Arg, Lys, His, Gln, and Asn;
X² is an amino acid chosen from Gly, Ser, Thr, Ala, Val, Leu, and Ile;
X³ is an amino acid chosen from Lys, Arg, and His;
X⁴ is an amino acid chosen from Asp, Glu, Asn, Gln, Ser, Thr, Tyr, Trp, Phe, Ala, and Gly.

In certain embodiments, the antibody or antibody fragment comprises

a VL CDR2 having the amino acid sequence X¹AX²SLAS, wherein
X¹ is an amino acid chosen from Lys, Arg, and His;
X² is an amino acid chosen from Gly, Ser, Thr, Ala, Val, Leu, and Ile.

In certain embodiments, the antibody or antibody fragment comprises a VL CDR3 having the amino acid sequence QQYSXYPLT, wherein X is an amino acid chosen from Asn, Asp, Glu, and Gln.

In certain embodiments, the antibody or antibody fragment of any of claims 36-39, wherein the antibody or antibody fragment comprises:

a VL CDR1 having the amino acid sequence X¹ASEX²IYX³X⁴LA, wherein
X¹ is an amino acid chosen from Arg, Lys, His, Gln, and Asn;
X² is an amino acid chosen from Gly, Ser, Thr, Ala, Val, Leu, and Ile;
X³ is an amino acid chosen from Lys, Arg, and His;
X⁴ is an amino acid chosen from Asp, Glu, Asn, Gln, Ser, Thr, Tyr, Trp, Phe, Ala, and Gly;

a VL CDR2 having the amino acid sequence X¹AX²SLAS, wherein
X¹ is an amino acid chosen from Lys, Arg, and His;
X² is an amino acid chosen from Gly, Ser, Thr, Ala, Val, Leu, and Ile; and

a VL CDR3 having the amino acid sequence QQYSXYPLT, wherein X is an amino acid chosen from Asn, Asp, Glu, and Gln.

In certain embodiments, the antibody or antibody fragment comprises:

a VH CDR1 having the amino acid sequence XYAIS, wherein X is an amino acid chosen from Ser, Thr, Gly, Ala, Val, Leu, and Ile;

a VH CDR2 having the amino acid sequence GIIPX¹FX²TANYX³QKX⁴X⁵X⁶, wherein
X1 is an amino acid chosen from Ile, Ser, Thr, Gly, Ala, Val, and Leu;
X2 is an amino acid chosen from Asp, Asn, Glu, Gln, Gly, Ala, Val, Leu, Ile, Ser, Thr, and Pro;
X3 is an amino acid chosen from Ala, Ser, Thr, Gly, Ala, Val, Leu, and Ile;
X⁴ is an amino acid chosen from Phe, Leu, Ile, Ala, Ser, Thr, Gly, Ala, and Val;
X⁵ is an amino acid chosen from Gln, Asn, Arg, Lys, and His;
X⁶ is an amino acid chosen from Gly, Ser, Thr, Ala, Val, Leu, and Ile; and

a VH CDR3 having the amino acid sequence EX¹RQWLEGX²FDY, wherein
X¹ is an amino acid chosen from Lys, Arg, and His;
X² is an amino acid chosen from Lys, Arg, and His.

In certain embodiments, the antibody or antibody fragment comprises:

a VH CDR1 having the amino acid sequence XYAIS, wherein X is an amino acid chosen from Ser and Gly.

In certain embodiments, the antibody or antibody fragment comprises:

a VH CDR2 having the amino acid sequence GIIPX¹FX²TANYX³QKX⁴X⁵X⁶, wherein
X1 is an amino acid chosen from Ile, Thr, and Ala;
X2 is an amino acid chosen from Asp, Gly, Val, Ser, and Pro;
X3 is an amino acid chosen from Ala and Ser;
X⁴ is an amino acid chosen from Phe and Leu;
X⁵ is an amino acid chosen from Gln and Arg;
X⁶ is an amino acid chosen from Gly and Ser.

In certain embodiments, the antibody or antibody fragment comprises:

a VH CDR3 having the amino acid sequence EX¹RQWLEGX²FDY, wherein
X¹ is an amino acid chosen from Lys and Arg;
X² is an amino acid chosen from Arg, and His.

In certain embodiments, the antibody or antibody fragment comprises:

a VL CDR1 having the amino acid sequence X¹AX³X⁴LA, wherein
X¹ is an amino acid chosen from Arg and Gln;
X² is an amino acid chosen from Gly and Ser;
X³ is an amino acid chosen from Arg and His;
X⁴ is an amino acid chosen from Glu, Asn, Gln, Ser, Thr, Trp, Phe, and Ala.

In certain embodiments, the antibody or antibody fragment comprises:

a VL CDR2 having the amino acid sequence X¹AX²SLAS, wherein
X¹ is an amino acid chosen from Lys and Arg;
X² is an amino acid chosen from Ser and Thr.

In certain embodiments, the antibody or antibody fragment comprises:

a VL CDR3 having the amino acid sequence QQYSXYPLT, wherein X is an amino acid chosen from Asn and Asp.

In another embodiment, the human or chimeric antibody or antibody fragment for use in the claimed methods immunospecifically binds to human PAI-1, inhibits human PAI-1 activity and comprises:

a VH CDR1 having the amino acid sequence XYAIS, wherein X is a neutral amino acid residue;

a VH CDR2 having the amino acid sequence GIIPX¹FX²TANYX³QKX⁴X⁵X⁶, wherein
X1 is a neutral amino acid residue;
X2 is a neutral or acidic amino acid residue;
X3 is a neutral amino acid residue;
X⁴ is a neutral amino residue;
X⁵ is a neutral or basic amino acid residue;
X⁶ is a neutral amino acid residue;

a VH CDR3 having the amino acid sequence EX¹RQWLEGX²FDY, wherein
X¹ is a basic amino acid residue;
X² is a basic amino acid residue;

a VL CDR1 having the amino acid sequence X¹ASEX²IYX³X⁴LA, wherein
X¹ is a basic or neutral amino acid residue;
X² is a neutral amino acid residue;
X³ is a basic amino acid residue;
X⁴ is an acidic or neutral amino acid residue;

a VL CDR2 having the amino acid sequence X¹AX²SLAS, wherein
X¹ is a basic amino acid residue;
X² is a neutral amino acid residue; and

a VL CDR3 having the amino acid sequence QQYSXYPLT, wherein X is an acidic or neutral amino acid residue.

In certain embodiments, the antibody or antibody fragment comprises

a VH CDR1 having the amino acid sequence XYAIS, wherein X is an amino acid chosen from Ser, Thr, Gly, Ala, Val, Leu, and Be;

a VH CDR2 having the amino acid sequence GIIPX¹FX²TANYX³QKX⁴X⁵X⁶, wherein
X1 is an amino acid chosen from Ile, Ser, Thr, Gly, Ala, Val, and Leu;
X2 is an amino acid chosen from Asp, Asn, Glu, Gln, Gly, Ala, Val, Leu, Ile, Ser, Thr, and Pro;
X3 is an amino acid chosen from Ala, Ser, Thr, Gly, Ala, Val, Leu, and Ile;
X⁴ is an amino acid chosen from Phe, Leu, Ile, Ala, Ser, Thr, Gly, Ala, and Val;
X⁵ is an amino acid chosen from Gln, Asn, Arg, Lys, and His;
X⁶ is an amino acid chosen from Gly, Ser, Thr, Ala, Val, Leu, and Ile;

a VH CDR3 having the amino acid sequence EX¹RQWLEGX²FDY, wherein
X¹ is an amino acid chosen from Lys, Arg, and His;
X² is an amino acid chosen from Lys, Arg, and His;

a VL CDR1 having the amino acid sequence X¹ASEX²IYX³X⁴LA, wherein
X¹ is an amino acid chosen from Arg, Lys, His, Gln, and Asn;
X² is an amino acid chosen from Gly, Ser, Thr, Ala, Val, Leu, and Ile;
X³ is an amino acid chosen from Lys, Arg, and His;
X⁴ is an amino acid chosen from Asp, Glu, Asn, Gln, Ser, Thr, Tyr, Trp, Phe, Ala, and Gly;

a VL CDR2 having the amino acid sequence X¹AX²SLAS, wherein
X¹ is an amino acid chosen from Lys, Arg, and His;
X² is an amino acid chosen from Gly, Ser, Thr, Ala, Val, Leu, and Ile; and

a VL CDR3 having the amino acid sequence QQYSXYPLT, wherein X is an amino acid chosen from Asn, Asp, Glu, and Gln.

In certain embodiments, the antibody or antibody fragment comprises

a VH CDR1 having the amino acid sequence XYAIS, wherein X is an amino acid chosen from Ser and Gly;

a VH CDR2 having the amino acid sequence GIIPX¹FX²TANYX³QKX⁴X⁵X⁶, wherein
X1 is an amino acid chosen from Ile, Thr, and Ala;
X2 is an amino acid chosen from Asp, Gly, Val, Ser, and Pro;
X3 is an amino acid chosen from Ala and Ser;
X⁴ is an amino acid chosen from Phe and Leu;
X⁵ is an amino acid chosen from Gln and Arg;
X⁶ is an amino acid chosen from Gly and Ser;

a VH CDR3 having the amino acid sequence EX¹RQWLEGX²FDY, wherein
X¹ is an amino acid chosen from Lys and Arg;
X² is an amino acid chosen from Arg, and His;

a VL CDR1 having the amino acid sequence X¹ASEX²IYX³X⁴LA, wherein
X¹ is an amino acid chosen from Arg and Gln;
X² is an amino acid chosen from Gly and Ser;
X³ is an amino acid chosen from Arg and His;
X⁴ is an amino acid chosen from Glu, Asn, Gln, Ser, Thr, Trp, Phe, and Ala;

a VL CDR2 having the amino acid sequence X¹AX²SLAS, wherein
X¹ is an amino acid chosen from Lys and Arg;
X² is an amino acid chosen from Ser and Thr;

a VL CDR3 having the amino acid sequence QQYSXYPLT, wherein X is an amino acid chosen from Asn and Asp.

In certain embodiments, the antibody or antibody fragment comprises:

a VH CDR1 having the amino acid sequence XYAIS, wherein X is a neutral amino acid residue;

a VH CDR2 having the amino acid sequence GIIPX¹FX²TANYX³QKX⁴X⁵X⁶, wherein
X1 is a neutral amino acid residue;
X2 is a neutral or acidic amino acid residue;
X3 is a neutral amino acid residue;
X⁴ is a neutral amino residue;
X⁵ is a neutral or basic amino acid residue;
X⁶ is a neutral amino acid residue;

a VH CDR3 having the amino acid sequence EX¹RQWLEGX²FDY, wherein
X¹ is a basic amino acid residue;
X² is a basic amino acid residue;

a VL CDR1 having the amino acid sequence X¹ASEX²IYX³X⁴LA, wherein
X¹ is a basic or neutral amino acid residue;
X² is a neutral amino acid residue;
X³ is a basic amino acid residue;
X⁴ is an acidic or neutral amino acid residue;

a VL CDR2 having the amino acid sequence X¹AX²SLAS, wherein
X¹ is a basic amino acid residue;
X² is a neutral amino acid residue; and

a VL CDR3 having the amino acid sequence QQYSXYPLT, wherein X is an acidic or neutral amino acid residue.

In certain embodiments, the antibody or antibody fragment comprises

a VH CDR1 having the amino acid sequence XYAIS, wherein X is an amino acid chosen from Ser, Thr, Gly, Ala, Val, Leu, and Be;

a VH CDR2 having the amino acid sequence GIIPX¹FX²TANYX³QKX⁴X⁵X⁶, wherein
X1 is an amino acid chosen from Ile, Ser, Thr, Gly, Ala, Val, and Leu;
X2 is an amino acid chosen from Asp, Asn, Glu, Gln, Gly, Ala, Val, Leu, Ile, Ser, Thr, and Pro;
X3 is an amino acid chosen from Ala, Ser, Thr, Gly, Ala, Val, Leu, and Ile;
X⁴ is an amino acid chosen from Phe, Leu, Ile, Ala, Ser, Thr, Gly, Ala, and Val;
X⁵ is an amino acid chosen from Gln, Asn, Arg, Lys, and His;
X⁶ is an amino acid chosen from Gly, Ser, Thr, Ala, Val, Leu, and Ile;

a VH CDR3 having the amino acid sequence EX¹RQWLEGX²FDY, wherein
X¹ is an amino acid chosen from Lys, Arg, and His;
X² is an amino acid chosen from Lys, Arg, and His;

a VL CDR1 having the amino acid sequence X¹ASEX²IYX³X⁴LA, wherein
X¹ is an amino acid chosen from Arg, Lys, His, Gln, and Asn;
X² is an amino acid chosen from Gly, Ser, Thr, Ala, Val, Leu, and Ile;
X³ is an amino acid chosen from Lys, Arg, and His;
X⁴ is an amino acid chosen from Asp, Glu, Asn, Gln, Ser, Thr, Tyr, Trp, Phe, Ala, and Gly;

a VL CDR2 having the amino acid sequence X¹AX²SLAS, wherein
X¹ is an amino acid chosen from Lys, Arg, and His;
X² is an amino acid chosen from Gly, Ser, Thr, Ala, Val, Leu, and Ile; and

a VL CDR3 having the amino acid sequence QQYSXYPLT, wherein X is an amino acid chosen from Asn, Asp, Glu, and Gln.

In certain embodiments, the antibody or antibody fragment comprises

a VH CDR1 having the amino acid sequence XYAIS, wherein X is an amino acid chosen from Ser and Gly;

a VH CDR2 having the amino acid sequence GIIPX¹FX²TANYX³QKX⁴X⁵X⁶, wherein
X1 is an amino acid chosen from Ile, Thr, and Ala;
X2 is an amino acid chosen from Asp, Gly, Val, Ser, and Pro;
X3 is an amino acid chosen from Ala and Ser;
X⁴ is an amino acid chosen from Phe and Leu;
X⁵ is an amino acid chosen from Gln and Arg;
X⁶ is an amino acid chosen from Gly and Ser;

a VH CDR3 having the amino acid sequence EX¹RQWLEGX²FDY, wherein
X¹ is an amino acid chosen from Lys and Arg;
X² is an amino acid chosen from Arg, and His;

a VL CDR1 having the amino acid sequence X¹ASEX²IYX³X⁴LA, wherein
X¹ is an amino acid chosen from Arg and Gln;
X² is an amino acid chosen from Gly and Ser;
X³ is an amino acid chosen from Arg and His;
X⁴ is an amino acid chosen from Glu, Asn, Gln, Ser, Thr, Trp, Phe, and Ala;

a VL CDR2 having the amino acid sequence X¹AX²SLAS, wherein
X¹ is an amino acid chosen from Lys and Arg;
X² is an amino acid chosen from Ser and Thr;

a VL CDR3 having the amino acid sequence QQYSXYPLT, wherein X is an amino acid chosen from Asn and Asp.

In another embodiment, the human or chimeric antibody or antibody fragment for use in the claimed method immunospecifically binds to human PAI-1, inhibits human PAI-1 activity and comprises:

a VH CDR1 having an amino acid sequence chosen from: SEQ ID NOs: 113 and 159;

a VH CDR2 having an amino acid sequence chosen from: SEQ ID NOs: 114 and 160-169;

a VH CDR3 having an amino acid sequence chosen from: SEQ ID NOs: 115 and 170-171;

a VL CDR1 having an amino acid sequence chosen from: SEQ ID NOs: and 172-182;

a VL CDR2 having an amino acid sequence chosen from: SEQ ID NOs: 117 and 183-184; and

a VL CDR3 having an amino acid sequence chosen from: SEQ ID NOs: 118 and 185.

In certain embodiments, the antibody or antibody fragment comprises:

a VH CDR1 having the amino acid sequence of SEQ ID NO: 113;

a VH CDR2 having the amino acid sequence of SEQ ID NO: 161;

a VH CDR3 having the amino acid sequence of SEQ ID NO: 171;

a VL CDR1 having the amino acid sequence of SEQ ID NO: 176;

a VL CDR2 having the amino acid sequence of SEQ ID NO: 117; and

a VL CDR3 having the amino acid sequence of SEQ ID NO: 186.

In certain embodiments, the three VH CDRs comprise:

a VH CDR1 having an amino acid sequence chosen from: SEQ ID NOs: 113 and 159;

a VH CDR2 having an amino acid sequence chosen from: SEQ ID NOs: 114 and 160-169; and

a VH CDR3 having an amino acid sequence chosen from: SEQ ID NOs: 115 and 170-171.

In certain embodiments, the three VH CDRs comprise:

a VH CDR1 comprising the amino acid sequence of SEQ ID NO: 113;

a VH CDR2 comprising the amino acid sequence of SEQ ID NO: 161; and

a VH CDR3 comprising the amino acid sequence of SEQ ID NO: 171.

In certain embodiment, the three VL CDRs comprise:

a VL CDR1 having an amino acid sequence chosen from: SEQ ID NOs: 172-182;

a VL CDR2 having an amino acid sequence chosen from: SEQ ID NOs: 117 and 183-184; and

a VL CDR3 having an amino acid sequence chosen from: SEQ ID NOs: 118 and 185.

In certain embodiments, the three VL CDRs comprise:

a VL CDR1 comprising the amino acid sequence of SEQ ID NO: 176;

a VL CDR2 comprising the amino acid sequence of SEQ ID NO: 117; and

a VL CDR3 comprising the amino acid sequence of SEQ ID NO: 186.

In another embodiment, the human or chimeric antibody or antibody fragment, immunospecifically binds human PAI-1, inhibits human PAI-1 activity and comprises

a heavy chain variable domain (VH) at least 85% identical to the amino acid of SEQ ID NO: 6 and
a light chain variable domain (VL) at least 90% identical to the amino acid sequence of SEQ ID NO: 8.

In certain embodiments, the antibody or antibody fragment comprises:

a VH domain comprising an amino acid sequence chosen from: SEQ ID NOs: 6, 10, 14, 18, 22, 26, 30, 34, 38, 42, 46, 50, 54, 58, 62, 66, 70, 74, 78, 82, 86, 90, 94, 98, 102, 106, and 110; and

a VL domain comprising an amino acid sequence chosen from: SEQ ID NOs: 8, 12, 16, 20, 24, 28, 32, 36, 40, 44, 48, 52, 56, 60, 64, 68, 72, 76, 80, 84, 88, 92, 96, 100, 104, 108, and 112.

In certain embodiments, the antibody or antibody fragment comprises: (a) a VH comprising the amino acid sequence of SEQ ID NO: 34; and (b) a VL comprising the amino acid sequence of SEQ ID NO: 36.

In certain embodiments, the antibody or antibody fragment of the disclosure has the following feature: Kabat residue 66 of the VL domain framework is Arg.

In certain embodiments, the human or chimeric antibody or antibody fragment for use in the claimed methods immunospecifically binds to human PAI-1, inhibits human PAI-1 activity and comprises:

a VH CDR1 having the amino acid sequence of SEQ ID NO: 187;

a VH CDR2 having the amino acid sequence of SEQ ID NO: 161;

a VH CDR3 having an amino acid sequence identical to or comprising 1 or 2 amino acid residue substitutions relative to SEQ ID NO: 171;

a VL CDR1 having the amino acid sequence of SEQ ID NO: 176;

a VL CDR2 having an amino acid sequence identical to or comprising 1 or 2 amino acid residue substitutions relative of SEQ ID NO: 117; and

a VL CDR3 having an amino acid sequence identical to or comprising 1 or 2 amino acid residue substitutions relative to SEQ ID NO: 186.

For example, in certain embodiments, the antibody or antibody fragment comprises:

a VH CDR3 having the amino acid sequence EX¹RQWLEGX²FDY, wherein
X¹ is a basic amino acid residue;
X² is a basic amino acid residue.

By way of further example, in certain embodiments, the antibody or antibody fragment comprises:

a VL CDR2 having the amino acid sequence XASSLAS, wherein X is a basic amino acid residue; and

a VL CDR3 having the amino acid sequence QQYSXYPLT, wherein X is an acidic or neutral amino acid residue.

In certain embodiments, the human or chimeric antibody or antibody fragment for use in the claimed methods immunospecifically binds to human PAI-1, inhibits human PAI-1 activity and comprises:

a VH CDR1 having the amino acid sequence of SEQ ID NO: 187;

a VH CDR2 having an amino acid sequence identical to or comprising 1, 2 or 3 amino acid residue substitutions relative to SEQ ID NO: 161;

a VH CDR3 having the amino acid sequence of SEQ ID NO: 171;

a VL CDR1 having the amino acid sequence of SEQ ID NO: 176;

a VL CDR2 having an amino acid sequence identical to or comprising 1 or 2 amino acid residue substitutions relative to SEQ ID NO: 117; and

a VL CDR3 having the amino acid sequence of SEQ ID NO: 186.

For example, in certain embodiments, the antibody or antibody fragment comprises:

a VH CDR2 having the amino acid sequence GIIPX¹FX²TANYAQKFX³G, wherein
X¹ is a neutral amino acid residue;
X² is an acidic or neutral amino acid residue;
X³ is a basic or neutral amino acid residue.

By way of further example, in certain embodiments, the antibody or antibody fragment comprises:

a VL CDR2 having the amino acid sequence XASSLAS, wherein X is a basic amino acid residue.

In certain other embodiments, the human or chimeric antibody or antibody fragment for use in the claimed methods immunospecifically binds to human PAI-1, inhibits human PAI-1 activity and comprises:

a VH CDR1 having the amino acid sequence of SEQ ID NO: 187;

a VH CDR2 having the amino acid sequence of SEQ ID NO: 161;

a VH CDR3 having an amino acid sequence identical to or comprising 1 or 2 amino acid residue substitutions relative to SEQ ID NO: 171;

a VL CDR1 having an amino acid sequence identical to or comprising 1 or 2 amino acid residue substitutions relative SEQ ID NO: 176;

a VL CDR2 having the amino acid sequence of SEQ ID NO: 117; and

a VL CDR3 having the amino acid sequence of SEQ ID NO: 186.

In certain other embodiments, the human or chimeric antibody or antibody fragment for use in the claimed methods immunospecifically binds to human PAI-1, inhibits human PAI-1 activity and comprises:

a VH CDR1 having the amino acid sequence of SEQ ID NO: 187;

a VH CDR2 having the amino acid sequence of SEQ ID NO: 161;

a VH CDR3 having the amino acid sequence of SEQ ID NO: 171;

a VL CDR1 having the amino acid sequence of SEQ ID NO: 176;

a VL CDR2 having the amino acid sequence of SEQ ID NO: 117; and

a VL CDR3 having the amino acid sequence of SEQ ID NO: 186.

In certain embodiments of any of the foregoing, the antibody or antibody fragment is an isolated antibody or antibody fragment. In other embodiments, the antibody or antibody fragment is a purified antibody or antibody fragment.

In certain embodiments, the antibody or antibody fragment comprises one or more amino acid substitutions, and each such amino acid substitution is a conservative substitution.

In certain embodiments, the antibody or antibody fragment inhibits binding of human PAI-1 to uPA.

In certain embodiments, the antibody or antibody fragment can reduce the level of VCAM-1 in dermal tissues; can reduce the level of TNF-alpha in dermal tissues; and can stimulate plasmin-mediated activation of MMP-1.

In certain embodiments, the antibody or antibody fragment is a human or chimeric antibody or antibody fragment that immunospecifically binds to human PAI-1 and inhibits PAI-1 activity, and has at least one or more (e.g., at least 1, 2, 3) of the following characteristics:

affinity (K_{D}) between 5 pm to 200 pM for active human PAI-1, as assessed by surface plasmon resonance;

immunospecifically binds to a human PAI-1:vitronectin complex, but does not disrupt the binding of human PAI-1 to vitronectin;

specifically binds to the reactive center loop of PAI-1 that interacts with tPA and uPA.

In certain embodiments, the antibody or antibody fragment has an affinity (K_{D}) of between about 5 pM and 125 pM for active human PAI-1, as assessed by surface plasmon resonance.

In certain embodiments, the antibody or antibody fragment has an affinity (K_{D}) of less than or equal to about 100 pM for active human PAI-1, as assessed by surface plasmon resonance.

In certain embodiments, the antibody or antibody fragment has an affinity (K_{D}) of less than or equal to 50 pM for active human PAI-1, as assessed by surface plasmon resonance

The following features describe certain embodiments of any of the foregoing aspects and embodiments of the disclosure. In other words, the following features describe certain embodiments that may describe any antibodies for use in the claimed methods disclosure.

In certain embodiments, the antibody is a human antibody. In certain embodiments, the antibody is a monoclonal antibody. In certain embodiments, the antibody is an antibody fragment. In certain embodiments, an amino acid substitution is a conservative substitution. In certain embodiments, an amino acid substitution is a non-conservative substitution.

In certain embodiments, the antibody or antibody fragment inhibits human PAI-1 activity by inhibiting binding of human PAI-1 to tPA by at least 50%. In certain embodiments, the antibody or antibody fragment does not immunospecifically bind to human PAI-2 or human PAI-3. In certain embodiments, the antibody or antibody fragment immunospecifically binds to human PAI-1 and also immunospecifically binds to mouse PAI-1 and/or rat PAI-1. In certain embodiments, the antibody or antibody fragment immunospecifically binds to human PAI-1 and also immunospecifically binds to PAI-1 from one or more non-human primates. In certain embodiments, the antibody or antibody fragment immunospecifically binds to human PAI-1 but does not immunospecifically bind to mouse PAI-1. In certain embodiments, the antibody or antibody fragment immunospecifically binds to human PAI-1 with an affinity at least one order of magnitude greater than its affinity for mouse PAI-1.

In certain embodiments, the antibody or antibody fragment can reduce the level of VCAM-1 in dermal tissues; can reduce the level of TNF-alpha in dermal tissues; and can stimulate plasmin-mediated activation of MMP-1.

In certain embodiments, the antibody or antibody fragment is a human or chimeric antibody or antibody fragment that immunospecifically binds to human PAI-1 and inhibits PAI-1 activity, and has at least one or more (e.g., at least 1, 2, 3) of the following characteristics:

affinity (K_{D}) between 5 pm to 200 pM for active human PAI-1, as assessed by surface plasmon resonance;

immunospecifically binds to a human PAI-1:vitronectin complex, but does not disrupt the binding of human PAI-1 to vitronectin;

specifically binds to the reactive center loop of PAI-1 that interacts with tPA and uPA.

In certain embodiments, the antibody or antibody fragment has an affinity (K_{D}) of between about 5 pM and 125 pM for active human PAI-1, as assessed by surface plasmon resonance.

In certain embodiments, the antibody or antibody fragment has an affinity (K_{D}) of less than or equal to about 100 pM for active human PAI-1, as assessed by surface plasmon resonance.

In certain embodiments, the antibody or antibody fragment has an affinity (K_{D}) of less than or equal to 50 pM for active human PAI-1, as assessed by surface plasmon resonance.

In one embodiment, the antibody or antibody fragment for use in the claimed methods is not an scFv.

In another embodiment the antibody or antibody fragment for use in the claimed methods has any combination of CDRs set forth in Table 2 and/or the Sequence Listing. In another embodiment, the antibody or antibody fragment has at least 3 (e.g., 3, 4, 5, or 6) of the CDRs of any antibody set forth in Table 2 and/or the Sequence Listing. In another embodiment, the antibody or antibody fragment has at least the VH and/or VL of an antibody set forth in Table 2, 16 and/or the Sequence Listing.

In another embodiment, the antibody is a full length antibody in an IgG format.

In another aspect, the disclosure provides a sterile composition comprising any of the antibodies of the disclosure (e.g., an antibody having any combination of the structural and functional features described herein).

In another aspect, the disclosure provides a composition comprising an antibody or antibody fragment of the disclosure, formulated with a pharmaceutically acceptable carrier. In one embodiments, such a composition is a sterile composition. In another embodiments, such a composition (which may be a sterile composition) is a pyrogen-free composition.

In another aspect, the disclosure provides a purified or isolated polypeptide, comprising the amino acid sequence of dog PAI-1. For, example, a polypeptide comprising the amino acid sequence of SEQ ID NO: A. In certain embodiments, the polypeptide consists of the amino acid sequence of SEQ ID NO: A. In certain embodiments, the polypeptide is glycosylated. In certain embodiments, the polypeptide is glycosylated in a manner that differs from the native glycosylation pattern of the naturally occurring polypeptide.

In another aspect, the disclosure provides a purified or isolated polypeptide, comprising the amino acid sequence of cynomolgous PAI-1. For example, the purified or isolated polypeptide comprises the amino acid sequence of SEQ ID NO: B or SEQ ID NO: C, In certain embodiments, the polypeptide consists of the amino acid sequence of SEQ ID NO: B or SEQ ID NO: C. In one embodiment, the polypeptide is glycosylated. In another embodiment, the polypeptide is glycosylated in a manner that differs from the native glycosylation pattern of the naturally occurring polypeptide.

In another aspect, the disclosure provides a purified or isolated complex, comprising a dog PAI-1 and a vitronectin polypeptide. For example, a purified or isolated complex comprising a dog PAI-1 polypeptide having the amino acid sequence of SEQ ID NO:A and a vitronectin polypeptide. The dog PAI-1 polypeptide may, in certain embodiments, be glycosylated in a way that is the same or differs from that of the native glycosylation pattern on the naturally occurring polypeptide. In certain embodiments, the vitronect polypeptide is a dog or human vitronectin polypeptide.

In another aspect, the disclosure provides a purified or isolated complex, comprising a cynomolgus PAI-1 and a vitronectin polypeptide. For example, a purified or isolated complex comprising a cynomolgus PAI-1 polypeptide having the amino acid sequence of SEQ ID NO:B or C and a vitronectin polypeptide. The cynomolgus PAI-1 polypeptide may, in certain embodiments, be glycosylated in a way that is the same or differs from that of the native glycosylation pattern on the naturally occurring polypeptide. In certain embodiments, the vitronectin polypeptide is a cynomolgus or human vitronectin polypeptide.

In another aspect, the disclosure provides methods of making dog or cynomolgus polypeptides or complexes comprising such polypeptides. The method comprising providing a host cell comprising a vector including a nucleic acid encoding such a polypeptide. The host cell is capable of expressing the polypeptide encoded by the nucleic acid in the vector. The method further comprises culturing the host cell under suitable conditions, such that the PAI-1 polypeptide is expressed. In certain embodiments, the method further comprises purifying the PAI-1 polypeptide.

The disclosure contemplates all combinations of the foregoing aspects and embodiments of the disclosure, including combinations based on features and embodiments set forth below and in the examples. Antibodies of the disclosure can be described based on any of the structural and functional features of anti-PAI-1 antibodies set forth above and below. Moreover, any such antibodies can be used in any of the methods of the disclosure, including in vivo and ex vivo. Sequence information for exemplary antibodies are set forth in the tables. Specifically contemplated for use in the claimed methods are anti-PAI-1 antibodies having the amino acid sequence of 1, 2, 3, 4, 5, or all 6 CDRs of any one or more of such antibodies, as well as antibodies having the VH and/or VL chain of any one or more of such antibodies.

### BRIEF DESCRIPTION OF THE FIGURES

FIG. 1: Reaction of PAI-1 with target protease (uPA is represented). Mechanisms of PAI-1 inhibition are: (i) acceleration of transformation from the active to latent conformation of PAI-1; (ii) prevention of formation of the initial (Michaelis-Menten) complex between PAI-1 and its target proteinases ([PAI-1:uPA]_{M}); and (iii) conversion of PAI-1 from a suicide inhibitor to a substrate for its target proteinases.

FIG. 2: Equipotent inhibition of human, cynomolgus and rodent PAI-1 activity by anti-PAI-1 scFvs. PAI-1 activity is shown as % of total activity in the absence of inhibiting antibody. scFv concentrations are shown in M on a logarithmic scale. The data for human, cynomolgus, rat and mouse PAI-1 are shown in FIGS. 2a, 2b, 2c and 2d, respectively. Anti-PAI-1 scFvs shown are, Antibody 08, Antibody 16 and an irrelevant control antibody.

FIG. 3: Example IC50 data for PICK167_A01 in the human, cynomolgus and rat PAI-1: tPA functional assays (3839/05 p68-75).

FIG. 4: Effect of lead scFv clone PICK167_A01 against 10 nM human, 15 nM mouse and 15 nM rat PAI-1 in the HT1080 plasminogen activation assay.

FIG. 5: IgG competition ELISA data on PICK167_A01. (a) PICK167_A01 cross reacts with other PAI-1 species; (b) PICK167_A01 is specific for PAI-1 above other serpins; (c) Tabulated data, with numbers showing fold difference in observed binding KD of PICK167_A01 IgG1 to PAI-1 variants compared to human wild-type active.

FIG. 6: Example IC50 data for PICK167_A01 IgG1 in the human, cynomolgus and rat PAI-1: tPA functional assays (3839/05 p171-176).

FIG. 7: IgG competition ELISA data. (a) PICK167_A01-fgl (CAT-1001) cross-reacts with cynomolgus monkey, rat and murine PAI-1; (b) PICK167_A01-fgl (CAT-1001) binds to both glycosylated and latent PAI-1; (c) PICK167_A01-fgl (CAT-1001) is specific for PAI-1 above other serpins; and (d) PICK167_A01-fgl (CAT-1001) IgG1 to PAI-1 variants compared to human wild-type active.

FIG. 8: Example IC50 data for PICK_{—}167A01-fgl IgG1 in the glycosylated human, cynomolgus and rat PAI-1: tPA functional assays.

FIG. 9: Inhibitory effect of 167_A01 FGL IgG1 (CAT-1001) against human (10 nM) and rat (15 nM) PAI-1 in a HT1080 plasminogen activation assay and mouse PAI-1 (15 nM) in a MLg plasminogen activation assay.

FIG. 10: Inhibition of human and rat PAI-1 activity by anti-PAI-1 variants with arginine or glycine at position 66 in VL in cell-free chromogenic assays. PAI-1 activity is shown as % of total activity in the absence of inhibiting antibody. scFv concentrations are shown in M on a logarithmic scale. The data for Antibody 08 as well as Antibody 08-R66KO (knock-out) variant on human and rat PAI-1 are shown in FIGS. 10a and 10b, respectively. FIGS. 10c and 10d depict PICK117B05, PICK117B05-R66KI (knock-in), Antibody 18 and Antibody 18-R66KI variants on human and rat PAI-1, respectively.

FIG. 11: Cross-reactivity of anti-PAI-1 antibodies to active PAI-1 species variants and selectivity over latent human PAI-1 and other SERPINs as measured by a competition binding assay. The competitor concentrations are shown in M on a logarithmic scale. Assay data for Antibody 16 on PAI-1 variants and other SERPINs is shown in FIGS. 11a and 11b, respectively.

FIG. 12: Light chain interactions of a stable mutant PAI-1 with Antibody 08. This interaction is mainly with regions of PAI-1 that are invariant between the active and latent form. PAI-1 structure is shown in light grey in the lower half of FIG. 12 and the antibody light chain is shown in black in the upper half of FIG. 12. Direct interactions are detailed in Table 10.

FIG. 13: Heavy chain interactions of a stable mutant PAI-1 with Antibody 08. This interaction is mainly with the reactive centre loop of PAI-1 that only exists in the active confirmation of PAI-1. PAI-1 structure is shown in light grey in the lower left hand corner of FIG. 13 and the antibody heavy chain in shown in black in the upper half of FIG. 13. Direct interactions are detailed in Table 10.

FIG. 14: Epitope comparison with rodent PAI-1. The epitope amino acids that differ between rodent and humanPAI-1 are Y220E, Y241F and E350T. They are indicated by a star (*) in the alignment shown in FIG. 14.

FIG. 15: An alignment of PICK167_A01 to germline sequences VH 1-69 (DP10) and VK L12a and closest matching J regions.

FIG. 16: PAI-1 expression in an Adv-interferon-alpha accelerated murine model of lupus nephritis. FIG. 16A shows results where NZBxNZW/F1 mice were infected with adenovirus-IFN-alpha or Ad Null (vector control). Proteinureia, a marker of renal damage, was rougly assessed via the dipstick method. FIG. 16B depicts the fold change in PAI-1 mRNA (by Fuidigm) from kidney as compared to mean of 3 Ad Null treated mice at the same time point. FIG. 16C depicts IHC which shows increased PAI-1 expression in glomerulus from Adv-IFN-alpha treated mouse as compared to Ad Null control (40× magnification, 6 weeks post-adenovirus infection). Distal tubules did not stain. MEDI-579 was used as the detection antibody.

FIG. 17: Active PAI-1 levels in plasma of Adv-interferon-alpha infected mice compared to control.

FIG. 18: MEDI-579 at 10 mg/kg reduces average proteinuria scores compared to CAT-002 control treated mice.

FIG. 19: Active PAI-1 levels in plasma of mice at 4 and 6 weeks. Plasma active PAI-1 levels were assessed by ELISA at 4 weeks post-infection (retro-orbital bleeds) and 6 weeks post-infection (terminal cardiac bleeds).

FIG. 20: Plasma PAI-1 activity following treatment with MEDI-579. MEDI-579 was present in glomeruli of mice at termination (6 weeks-4 weeks beyond detectability in plasma).

FIG. 21: Immunostaining and FACS analysis-spleen immune cell expansion is not inhibited following treatment with MEDI-579. MEDI-579 is not protecting via an effect on immune cells. No effect on pDC and B cells in the spleens of CAT-1001 treated mice compared to isotype controls.

FIGS. 22A and 22B: Proteinuria is inhibited and sodium excretion is normalized following treatment with muMEDI-579. MEDI-579 inhibits the development of proteinuria and normalizes sodium excrection in the urine.

FIG. 23: Active PAI-1 is inhibited in plasma and kidney following treatment with muMEDI-579.

FIG. 24: H&E histopathology score is inhibited in kidney following treatment with muMEDI-579 and correlates with proteinuria.

FIG. 25: Histopathology subscores in kidney following treatment with muMEDI-579.

FIG. 26: Expression of vWF (IHC) in kidney following treatment with muMEDI-579.

FIGS. 27a-27e: Genes modulated by muMEDI-579 treatment.

FIG. 28: Plasmin and active PAI-1 in plasma following single dose MEDI-579 (10 mg/kg ip). At 4 weeks post-infection (a time when PAI-1 is known to be up-regulated), mice received a single dose of MEDI-579 or isotype control at 10 mg/kg. Active PAI-1 (squares) and active plasmin (triangles) in the plasma and kidneys were assessed by ELISA at 48 hours, 1, 2 and 3 weeks post-dose.

FIG. 29: PAI-1 blockade by muMEDI-579 reduces clinical skin score in murine GVHD.

FIG. 30: Prophylactic PAI-1 inhibition reduces skin pathology in GVHD. Pre-treatment with muMEDI-579 reduces skin clinical and histopathology scores in a model of scleroderma.

FIG. 31: Histological skin score following a prophylactic dosing regimen (FIG. 31A) and picture (FIG. 31B).

FIG. 32: Histology skin score correlated with clinical skin score, validating these scoring systems in assessing disease activity in murine GVHD.

FIG. 33: Efficacy on the development of proteinuria following a prophylactic dosing regimen. FIG. 33A depicts the proteinuria score for single-dose studies, and FIG. 33B depicts the proteinuria score for the two-dose study.

FIG. 34: Diagram showing three key pathological components of murine GVDH in skin. Scleroderma-like murine GVDH develops dermal inflammation, fibrosis and an occlusive vasculopathy.

FIGS. 35A and 35B: Active PAI-1 in skin and plasma following 10 mg/kg prophylactic treatment.

FIG. 36: Prophylactic treatment with muMEDI-579 normalizes gene expression of fibrinolytic biomarkers in skin in murine GVDH.

FIG. 37: Effects on gene expression following prophylactic treatment.

FIG. 38: Prophylactic treatment with muMEDI-579 reduces expression of inflammation biomarkers in skin in murine GVDH.

FIG. 39: Prophylactic treatment with muMEDI-579 reduces expression of fibrosis biomarkers TIMP-1 and IL-33.

FIGS. 40A-40C: Hypothetical mechanism of action of muMEDI-579 on GVDH in skin.

FIG. 41: Therapeutic treatment with muMEDI-579 improves clinical skin score in murine GVDH.

FIGS. 42A-42B: Histological skin score following a therapeutic dosing regimen. Therapeutic treatment with muMEDI-579 improves histology skin score in murine GVDH.

FIGS. 43A-43C: Active PAI-1, plasmin and fibrin(ogen) following therapeutic treatment. Therapeutic treatment with muMEDI579 reduces levels of active PAI-1, active plasmin and fibrinogen proteins in skin in murine GVDH.

FIGS. 44A-44F: Effects on gene expression following therapeutic treatment.

FIG. 45: MEDI-579 stimulates plasmin-mediated MMP-1 activation in a dose dependent manner.

FIG. 46: MEDI-579 promotes ECM degradation in lung fibroblasts and kidney mesangial cells. When both MEDI-579 and wt-PAI-1 were added for 72 h, the inhibition of ECM degradation seen with wt-PAI-1 was reversed by MEDI-579 in a dose-dependent manner (*P<0.001 vs. control; # P<0.001 vs. wt-PAI-1 alone).

FIG. 47: Effect on plasma PAI-1 activity in rats given MEDI-579 (CAT-1001), CAT-002 and vehicle.

FIG. 48: Effect of fibrin dissolution by MEDI-579 and CAT-002.

FIG. 49: Active PAI-1 after treatment with MEDI-579 or CAT-002 given 5 minutes before batroxobin.

FIGS. 50A-50C: Establishment of diabetic nephropathy in uninephrectomized db/db mice. Urinary albuminuria is increased after the onset of diabetes and progresses steadily.

FIGS. 51A-51B: Treatment by muMEDI-579 leads to reduction in albuminuria. PAI-1R used as positive control.

FIGS. 52A-52B: Treatment with MEDI-579 changes body weight, HbA1c and plasma glucose levels.

FIGS. 53A-53E: MEDI-579 reduces transcription of many genes involved in fibrosis.

FIG. 54: PAI-1 contributes to the pathology of diabetes and diabetes nephropathy via multiple mechanisms.

FIG. 55: PK/PD human dose estimation summary. Translational simulation using the PK/PD model fitted to the monkey data.

FIG. 56: Disease progression PK/PD model. Active PAI-1 levels in the circulation remain BLQ. Estimated IC50 for effect on skin score is 0.5 mg/mL.

FIG. 57: Predicted saturation of CL in humans following MEDI-579 15 mg/kg IV Q4W or 4 mg/kg IV Q2W.

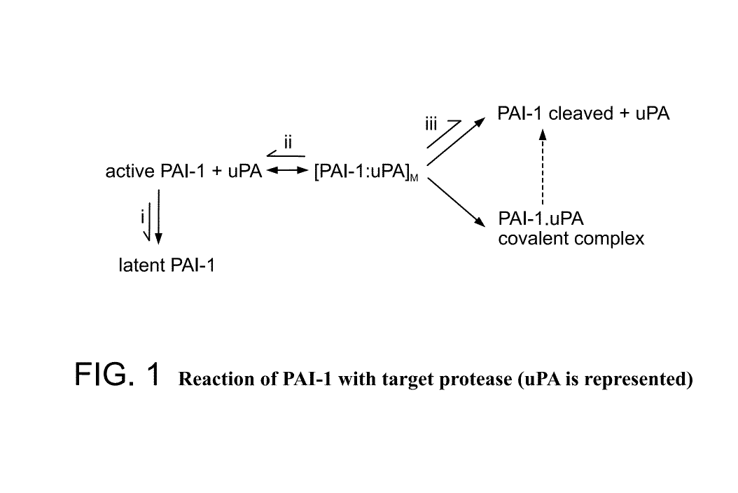

### DESCRIPTION OF TABLES

Table 1: List of coding polymorphisms in PAI-1 (SERPINE1).

Table 2: Alignment of CDR sequences of Antibody 1 to Antibody 27.

Table 3: IC50 of antibodies 1 to 27 in chromogenic assay.

Table 4: IC50 (nM) of selected antibodies in chromogenic assay with CHO cell-produced glycosylated human PAI-1.

Table 5: IC50 (nM) of selected antibodies in HT-1080 plasminogen activation assay with exogenous human, mouse and rat PAI-1.

Table 6: Binding kinetic and affinity data for Antibody 08 for human, cyno and rat PAI-1 as determined by BIAcore at 25° C. and at 37° C.

Table 7: Ratios of IC50 values of selected antibodies to glycosylated active human PAI-1, latent human PAI-1, rat PAI-1, mouse PAI-1 and rabbit PAI-1 in competition with non-glycosylated active human PAI-1 (“wt”).

Table 8: Top 10 binding peptides in PEPSCAN.

Table 9: Signal/background ratios for PEPSCAN peptides.

Table 10: PAI-1/Antibody 08 Fab direct interactions—table of epitope and paratope residues.

Table 11: Crystal parameters and X-ray data-processing and refinement statistics.

Table 12: PAI-1 epitope comparisons with Antibody 08, MAI-12 and H4B3.

Table 13: PAI-1 epitope comparisons of Antibody 08 with scFv-56A7C10.

Table 14: Epitope mapping showing residues present in the PAI-1/candidate antibody epitope (Antibody 08).

Table 15: Primer sequences for generation of PAI-1 mutant proteins as described in Example 11 (SEQ ID NOs: 202-212).

Table 16: List of SEQ ID NOs and description for each.

Table 17: List of key reagents used in assays.

Table 18: Additional list of key reagents used in assays.

### DETAILED DESCRIPTION

### A. Introduction

The present disclosure provides methods of treating various diseases and disorders using antibodies, including human, humanized and/or chimeric forms, as well as fragments, derivatives/conjugates and compositions thereof that immunospecifically bind to PAI-1. Anti-PAI-1 antibodies and antibody fragments are also referred to interchanageably herein as antibodies of the disclosure, anti-PAI-1 antibodies, and antibodies for use in the claimed methods. Diseases and conditions that can be treated include disease and conditions caused or exacerbated, in whole or in part, by fibrosis and/or inflammation. Exemplary diseases and conditions that can be treated include one or more of: systemic lupus erythromatosus (SLE), scleroderma, pulmonary fibrosis, wherein pulmonary fibrosis does not include IPF, diabetic nephropathy, lupus nephritis, graft versus host disease, glomerulonephritis, focal segmental glomerulosclerosis, membranous nephropathy, mesangial proliferative glomerulonephritis, membranoproliferative glomerulonephritis, renal fibrosis, and COPD.

PAI-1 belongs to the serine protease inhibitors (serpin) family. Serpins and their homologues are a group of high molecular weight (40 to 50 kDa) structurally related proteins involved in a number of fundamental biological processes, such as blood coagulation, complement activation, fibrinolysis, angiogenesis, inflammation, tumour suppression, and hormone transport. All known serpins have been classified into 16 clades and 10 orphan sequences; the vertebrate serpins can be conveniently classified into six sub-groups. In human plasma the serpins represent approximately 2% of the total protein, of which 70% is alpha-1-antitrypsin.

PAI-1 is a single-chain glycoprotein with an apparent molecular weight of 45 kDa. The protein consists of 379 or 381 amino acids due to N-terminal heterogeneity and has a 23 amino acid signal peptide indicating that it is a secreted protein.

The consensus coding sequence for the human PAI-1 gene, SERPINE1, is that of sequence NM_{—}000602 indicating that NM_{—}000602 (gi:10835158) represents the most common sequence (SEQ ID NO: 119 in the appended Sequence Listing). In addition to the coding changes, 5 polymorphisms were observed in the 5′ promoter region (including the 4G/5G polymorphism), 1 polymorphism was observed in the 5′ untranslated region (UTR), 12 polymorphisms were observed in the 3′UTR and a number of additional non-coding changes were also observed.

The identity of PAI-1 at the amino acid level across species is about 78 or 79% for mouse, about 80% for rat and about 86% for dog. The homology of PAI-1 to other members of the serpin family is relatively low. PAI-1 shares 25% identity with PAI-2 and 26% with PAI-3. The most closely related members of the serpin family are Protease nexin 1 (SERPINE2) which shows 37% identity with PAI-1, neuroserpin (SERPINI1) with 29% identity and pancpin (SERPINI2) with 28% identity.

PAI-1 is a fast-acting and highly selective inhibitor of target proteinases such as tPA and uPA. PAI-1 inhibits its target proteinases by formation of an initial 1:1 stoichiometric complex which is non-covalent and reversible. A covalent complex is then formed and PAI-1 is cleaved such that PAI-1 acts as a “suicide inhibitor”. PAI-1 has been identified in both active and latent conformations in vivo. In the active form, the reactive centre loop is exposed and the P1P1′ bond, comprising Arg346 and Met347, is able to interact with the target proteinases. Incubation of tPA or uPA with active PAI-1 leads to the formation of the covalent PAI-1:tPA/uPA complex upon the formation of an ester bond between the active site serine of tPA or uPA and the P1 residue of the PAI-1 reactive centre loop. However, the active form is unstable and spontaneously converts to the latent form with a half-life of 1-2 hours at 37° C. The crystal structures of active PAI-1 [i], latent PAI-1 [ii] and reactive centre cleaved PAI-1 [iii] have been solved. In certain embodiments of the present disclosure, a preferred antibody or antibody fragment is one that specifically binds to the reactive center loop of human PAI-1. In certain embodiments, the antibody or antibody fragment is only able to bind to the reactive center loop of human PAI-1 when PAI-1 is in the active form (e.g., when the reactive center loop is exposed). In certain embodiments, the antibody or antibody fragment for use in the claimed methods preferentially binds to active human PAI-1 over latent human PAI-1.

uPA is secreted from cells as a pro-enzyme. uPA catalyses the activation of plasminogen to plasmin, and active plasmin is responsible for degrading the fibrin component of clots. However, plasmin also activates a number of matrix metalloproteinases, which collectively can degrade extracellular matrix (ECM). Furthermore, uPA complexed with PAI-1 also binds uPAR but, unlike active uPA, this complex is rapidly internalised by the low-density lipoprotein (LDL) receptor-related protein (LRP). The uPA/PAI-1 complex is then degraded intra-cellularly and the uPAR recycled. tPA mediated plasminogen activation is mainly involved in the dissolution of fibrin in the circulation. uPA-catalyzed plasminogen activation has traditionally been associated with extracellular matrix degradation in the context of tissue remodeling, while tPA-catalyzed plasminogen activation has been traditionally associated with thrombolysis. In certain embodiments, antibodies or antibody fragments for use in the claimed methods can reduce the level of VCAM-1 in dermal tissues; can reduce the level of TNF-alpha in dermal tissues; and/or can stimulate plasmin-mediated activation of MMP-1.

Without being bound by theory, antibodies for use in the claimed methods of the disclosure are particularly useful for treating fibrotic conditions. Such antibodies are able to immunospecifically bind to human PAI-1, to inhibit PAI-1, and to stimulate plasmin-mediated activation of MMP-1. Such antibodies may also have one or more additional properties (function and/or structure) consistent with their use in the claimed methods.

PAI-1 is stabilized in the active conformation by binding to vitronectin [iv] and the high affinity binding region for PAI-1 in vitronectin has been mapped to the somatomedin B domain [v]. PAI-1 binding to vitronectin may compete with the uPAR— or integrin-dependent binding of cells to the extracellular matrix [vi]. Therefore, PAI-1 may have a role in cell adhesion and/or migration by a mechanism independent of its anti-proteolytic activity. Accordingly, it may be advantageous to select for use in vivo, antibodies that inhibit PAI-1 activity, such as the binding of PAI-1 to uPA and/or tPA, but which do not prevent or disrupt the binding of PAI-1 to vitronectin.

Native glycosylation of PAI-1 is biologically significant and it has been shown that glycosylated and non-glycosylated PAI-1 react differently to inactivating monoclonal antibodies and to solvent composition.

Small molecular weight inhibitors of PAI-1 have been reported in the literature. Tiplaxtinin, an indole acetic acid derivative [vii; viii] has been recently reported, and a derivative of this compound, PAI-749, is reported to be in Phase 1 development for thrombosis. Use of small molecule PAI-1 inhibitors for thrombosis have also been reported [ix; x].

The present disclosure provides antibodies and antibody fragments (antibodies of the disclosure) that specifically bind human PAI-1 and inhibit PAI-1 activity. In certain embodiments, the antibodies or antibody fragment have two or more Desired Characteristics, described in detail below. For example, antibodies of the disclosure specifically bind human PAI-1 and inhibit the binding of human PAI-1 to uPA, and/or the binding of human PAI-1 to tPA. Such antibodies may be used in the claimed methods. By inhibit the binding of human PAI-1 to uPA and/or tPA is meant that the antibody or antibody fragment inhibits binding by at least about 40%, 50%, 60%, 70%, 75%, 80%, 85% or at least 90%. In certain embodiments, binding is inhibited by greater than 90% (91%, 92%, 93%, 94%, 95%, etc.). Antibodies of the disclosure have use in, for example, in vitro or in vivo diagnostics, and for the treatment of disease or conditions caused in whole or in part by fibrosis and/or inflammation. Note that antibodies of the disclosure may also contribute to improvement in symptoms by having affects in addition to inhibiting fibrosis. Antibodies of the disclosure may have other functional or structural features, as described herein. Such features include, but are not limited to, affinity for human PAI-1, affinity for mouse PAI-1, affinity for cynomolgous PAI-1, amino acid sequence, and the like. The disclosure contemplates the use of antibodies and antibody fragments that specifically bind to human PAI-1 and have any one or more of the combinations of features described herein. The disclosure further contemplates that any such antibodies can be used in the diagnosis or treatment of the diseases and conditions described herein.

By way of example, in certain embodiments, antibodies (including fragments) of the disclosure may inhibit formation of the initial complex between PAI-1 and its target proteinase tPA and/or uPA. Inhibition of binding or complex formation by the antibodies may be direct inhibition. In certain embodiments, antibodies of the disclosure may optionally promote transformation or conversion of active PAI-1 to latent PAI-1.

Antibodies which bind to PAI-1, also referred to as anti-PAI-1 antibodies, are described herein. The antibodies bind and may neutralise (e.g., inhibit the activity) human PAI-1 and rodent (i.e., rat and/or mouse) PAI-1. The antibodies may bind and may neutralise rat, mouse and/or other rodent PAI-1, or lagomorph PAI-1, e.g., rabbit PAI-1, and/or non-human primate PAI-1 e.g., cynomolgus PAI-1, and/or canine PAI-1. Non-human PAI-1 refers to an ortholog of PAI-1 that occurs naturally in a species other than human.

In certain embodiments, the antibodies of the disclosure bind specifically to the reactive center loop of human PAI-1 that interacts with tPA and uPA. In certain embodiments, an antibody of the disclosure binds to the same epitope as any of the antibodies detailed in the Examples and Tables. In certain embodiments, an antibody of the disclosure binds to the same epitope as Antibody 8. In certain embodiments, an antibody of the disclosure binds to the same epitope as an antibody having the six CDRs of Antibody 8. In certain embodiments, an antibody of the disclosure binds the same epitope as an antibody having three CDRs of Antibody 8 variable heavy chain. In certain embodiments, an antibody of the disclosure competes with any of the antibodies detailed in the Examples, such as antibody 8, for binding to human PAI-1. Additional features of antibodies for use in the claimed methods are described herein.

The present disclosure provides methods for treating diseases and disorders caused or exacerbated, in whole or in part, by fibrosis. Anti-PAI-1 antibodies having the functional and. or structural features described herein are useful for treating disorders associated with PAI-1, especially fibrotic disorders, and they may be used for increasing fibrinolysis and/or increasing degradation of the extracellular matrix (ECM), e.g., reducing accumulation of ECM and/or fibrin. For example, an antibody of the disclosure may be used to treat scleroderma. Antibodies of the disclosure may also be used in treatment of other conditions, such as COPD, lupus nephritis, diabetic nephropathy, SLE, kidney fibrosis, etc.

As used herein, the terms “antibody” and “antibodies”, also known as immunoglobulins, encompass monoclonal antibodies (including full-length monoclonal antibodies), multispecific antibodies formed from at least two different epitope binding fragments (e.g., bispecific antibodies), human antibodies, humanized antibodies, camelised antibodies, chimeric antibodies, single-chain Fvs (scFv), single-chain antibodies, single domain antibodies, domain antibodies, Fab fragments, F(ab′)2 fragments, antibody fragments that exhibit the desired biological activity (e.g., the antigen binding portion), disulfide-linked Fvs (dsFv), and anti-idiotypic (anti-Id) antibodies (including, e.g., anti-Id antibodies to antibodies of the disclosure), intrabodies, and epitope-binding fragments of any of the above. In particular, antibodies include immunoglobulin molecules and immunologically active fragments of immunoglobulin molecules, i.e., molecules that contain at least one antigen-binding site. Immunoglobulin molecules can be of any isotype (e.g., IgG, IgE, IgM, IgD, IgA and IgY), subisotype (e.g., IgG1, IgG2, IgG3, IgG4, IgA1 and IgA2) or allotype (e.g., Gm, e.g., G1m (f, z, a or x), G2m (n), G3m (g, b, or c), Am, Em, and Km (1, 2 or 3)). Antibodies may be derived from any mammal, including, but not limited to, humans, monkeys, pigs, horses, rabbits, dogs, cats, mice, etc., or other animals such as birds (e.g., chickens).

Native antibodies are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains (CH). Each light chain has a variable domain at one end (VL) and a constant domain (CL) at its other end; the constant domain of the light chain is aligned with the first constant domain of the heavy chain, and the light chain variable domain is aligned with the variable domain of the heavy chain. Light chains are classified as either lambda chains or kappa chains based on the amino acid sequence of the light chain constant region. The variable domain of a kappa light chain may also be denoted herein as VK.

The antibodies for use in the claimed methods include full length or intact antibody, antibody fragments, native sequence antibody or amino acid variants, human, humanized, post-translationally modified, chimeric or fusion antibodies, immunoconjugates, and functional fragments thereof. The antibodies can be modified in the Fc region to provide desired effector functions or serum half-life. As discussed in more detail in the sections below, with the appropriate Fc regions, deleterious effects of effector function, such as antibody-dependent cytotoxicitiy and complement-dependent cytotoxicity, can be reduced or eliminated. The Fc region of the antibodies of the disclosure can be modified to increase the binding affinity for FcRn and thus increase serum half-life. Alternatively, the Fc region can be conjugated to PEG or albumin to increase the serum half-life, or some other conjugation that results in the desired effect.

In certain embodiments, the antibody competes for binding or bind substantially to, the same epitope as the antibodies of the disclosure. Antibodies having one or more biological characteristics (e.g., potency, PAI-1 affinity, effector function, ortholog binding affinity, neutralization, etc.) of the present anti-PAI-1 antibodies of the disclosure are also contemplated.

Functional and structural features of antibodies of the disclosure can be evaluated using any one of more of the assays described herein, as well as assays known in the art.

The disclosure provides a composition comprising an anti-PAI-1 antibody of the disclosure and a carrier. For the purposes of treating a disease or condition caused or exacerbated, in whole or in part, by fibrosis, compositions can be administered to the patient in need of such treatment, wherein the composition can comprise one or more anti-PAI-1 antibodies present, optionally, as an immunoconjugate or as the naked antibody. In a further embodiment, the method includes provides antibodies and compositions of the disclosure as part of a therapeutic regimen appropriate for the disease or condition being treated, such as in combination with steroids, anti-inflammatories, immunosuppresants, a dietary regimen, an exercise regimen, or stress management. The disclosure also provides formulations comprising an anti-PAI-1 antibody of the disclosure and a carrier. In one embodiment, the formulation is a therapeutic formulation comprising a pharmaceutically acceptable carrier. Antibodies for use in the treatment of a fibrotic disease or condition can be administered systemically or locally. Local administration includes via inhalation or intranasally for delivery to the lung.

In certain embodiments the disclosure provides methods useful for treating a PAI-1 associated fibrotic disease/condition and/or preventing and/or alleviating one or more symptoms of the PAI-1 associated fibrotic disease in a mammal, comprising administering a therapeutically effective amount of the anti-PAI-1 antibody to the mammal. The antibody therapeutic compositions can be administered short term (acute) or chronic, or intermittently as directed by a physician.

More details of the antibodies for use in the claimed methods are provided below. Antibodies having any combination of the structural and/or functional features described herein can be used in vitro or in vivo in any of the methods, including diagnostic and therapeutic methods, described herein.

### B. Terminology

Before describing the present disclosure in detail, it is to be understood that this disclosure is not limited to specific compositions or process steps, as such may vary. It must be noted that, as used in this specification and the appended claims, the singular form “a”, “an” and “the” include plural referents unless the context clearly dictates otherwise.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The numbering of amino acids in the variable domain, complementarity determining region (CDRs) and framework regions (FR), of an antibody follow, unless otherwise indicated, the Kabat definition as set forth in Kabat et al. Sequences of Proteins of Immunological Interest, 5th Ed. Public Health Service, National Institutes of Health, Bethesda, Md. (1991). Using this numbering system, the actual linear amino acid sequence may contain fewer or additional amino acids corresponding to a shortening of, or insertion into, a FR or CDR of the variable domain. For example, a heavy chain variable domain may include a single amino acid insertion (residue 52a according to Kabat) after residue 52 of H2 and inserted residues (e.g., residues 82a, 82b, and 82c, etc according to Kabat) after heavy chain FR residue 82. The Kabat numbering of residues may be determined for a given antibody by alignment at regions of homology of the sequence of the antibody with a “standard” Kabat numbered sequence. Maximal alignment of framework residues frequently requires the insertion of “spacer” residues in the numbering system, to be used for the Fv region. In addition, the identity of certain individual residues at any given Kabat site number may vary from antibody chain to antibody chain due to interspecies or allelic divergence.

### C. Anti-PAI-1 Antibodies

This section of the specification describes antibodies (and fragments) for use in the claimed methods. The disclosure contemplates the use of any such antibodies and fragments, described based on any combination of functional and/or structural features, in methods of inhibting fibrosis and/or treating any of the diseases and conditions described herein in a subject in need thereof.

In certain embodiments, the anti-PAI-1 antibodies are isolated and/or purified and/or pyrogen free antibodies. The term “purified” as used herein, refers to other molecules, e.g., polypeptide, nucleic acid molecule that have been identified and separated and/or recovered from a component of its environment. Thus, in one embodiment the antibodies of the disclosure are purified antibodies wherein they have been separated from one or more components of their natural environment. The term “isolated antibody” as used herein refers to an antibody which is substantially free of other antibody moleclues having different antigenic specificities (e.g., an isolated antibody that specifically binds to PAI-1 is substantially free of antibodies that specifically bind antigens other than PAI-1; however a bi- or multi-specific antibody molecule is an isolated antibody when substantially free of other antibody molecules). Thus, in one embodiment the antibodies of the disclosure are isolated antibodies wherein they have been separated from antibodies with a different specificity. Typically an isolated antibody is a monoclonal antibody. An isolated antibody that specifically binds to an epitope, isoform or variant of human PAI-1 may, however, have cross-reactivity to other related antigens, e.g., from other species (e.g., PAI-1 species homologs). Moreover, an isolated antibody of the disclosure may be substantially free of one or more other cellular materials and/or chemicals and is herein referred to an isolated and purified antibody. In one embodiment of the disclosure, a combination of “isolated” monoclonal antibodies relates to antibodies having different specificities and being combined in a well defined composition. Methods of production and purification/isolation of the anti-PAI-1 antibodies are described below in more detail.

Antibodies of the present disclosure include, in certain embodiments, antibody amino acid sequences disclosed herein encoded by any suitable polynucleotide, or any isolated or formulated antibody. Further, antibodies of the present disclosure comprise antibodies having the structural and/or functional features of anti-PAI-1 antibodies described herein. In one embodiment, the anti-PAI-1 antibody binds human PAI-1 and, thereby partially or substantially alters at least one biological activity of PAI-1 (e.g., binding, catalytic activity, etc.).

Anti-PAI-1 antibodies of the disclosure immunospecifically bind at least one specified epitope specific to the PAI-1 protein, peptide, subunit, fragment, portion or any combination thereof and do not specifically bind to other polypeptides, other than PAI-1 from other species. The at least one epitope can comprise at least one antibody binding region that comprises at least one portion of the PAI-1 protein. The term “epitope” as used herein refers to a protein determinant capable of binding to an antibody. Epitopes usually consist of chemically active surface groupings of molecules such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents.

In certain embodiments, the epitope is comprised of at least one extracellular, soluble, hydrophilic, external or cytoplasmic portion of the PAI-1. In certain embodiments, antibodies of the disclosure bind to the same epitope as Antibody 8. In certain embodiments, antibodies of the disclosure bind to the reactive loop of PAI-1 that mediates binding of PAI-1 to tPA and uPA.

In certain embodiments, antibodies for use in the claimed methods have, at least, the following characteristics: immunospecifically bind human PAI-1, inhibit PAI-1 activity, and can stimulate plasmin mediated activation of MMP-1. In certain embodiments, antibodies for use in the claimed methods have, at least, the following characteristics: immunospecifically bind human PAI-1, inhibit PAI-1 activity, can stimulate plasmin mediated activation of MMP-1, can reduce the level of VCAM-1 in dermal tissues; and can reduce the level of TNF-alpha in dermal tissues.

In certain embodiments, antibodies for use in the claimed methods have, at least, the following characteristics: immunospecifically bind human PAI-1, inhibit PAI-1 activity, and have at least one or more of the following characteristics:

(a) immunospecifically binds to a human PAI-1:vitronectin complex, but does not disrupt the binding of human PAI-1 to vitronectin;

(b) specifically binds to the reactive center loop of PAI-1 that interacts with tPA and uPA;

(c) immunospecifically binds to glycosylated human PAI-1;

(d) does not immunospecifically bind to human PAI-2 or human PAI-3;

(e) inhibits the binding of human PAI-1 to tPA by at least 50%;

(f) inhibits the binding of human PAI-1 to tPA with an IC₅₀ of about 15 nM or less;

(g) immunospecifically binds human PAI-1 and mouse PAI-1;

(h) immunospecifically binds human PAI-1, mouse PAI-1, and rat PAI-1;

(i) immunospecifically binds human PAI-1, mouse PAI-1, and PAI-1 from at least one non-human primate;

(j) affinity (K_{D}) between about 5 μM to 200 pM for active human PAI-1, as assessed by surface plasmon resonance;

(k) immunospecifically binds to a human PAI-1 epitope chosen from SEQ ID NOs: 156-158;

(l) immunospecifically binds to cynomolgus PAI-1 with approximately the same affinity as human PAI-1;

(m) immunospecifically binds to human PAI-1 with an affinity at least one order of magnitude greater than its affinity for mouse and/or rat PAI-1;

(n) preferentially binds to active human PAI-1 over latent human PAI-1;

(o) bind the same epitope as Antibody 8;

(p) can reduce the level of VCAM-1 in dermal tissues;

(q) can reduce the level of TNF-alpha in dermal tissues;

(r) can stimulate plasmin-mediated activation of MMP-1.

In certain embodiments, the antibody or antibody fragment has at least any number of these characteristics, such as at least 3, 4, 5, 6, 7, 8, 9, 10, 11, 12, 13, 14, 15, 16. Of course, such antibodies may optionally have additional characteristics.

In certain embodiments, antibodies or antibody fragments for use in the claimed methods have an affinity (K_{D}) between about 5 pM and 125 pM for active human PAI-1, as assessed by surface plasmon resonance. In certain embodiments, such antibodies have an affinity (K_{D}) of less than or equal to 100 pM for active human PAI-1, as assessed by surface plasmon resonance. In certain other embodiments, such antibodies have an affinity (K_{D}) of less than or equal to 50 pM for active human PAI-1, as assessed by surface plasmon resonance.

Exemplary antibodies are provided herein. Exemplary features of such exemplary antibodies are set forth in the Sequence Listing, tables, and examples.

1. Variable Regions

The antigen-binding portion of an antibody comprises one or more fragments of an antibody that retain the ability to specifically bind to an antigen (e.g., PAI-1). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

The present anti-PAI-1 antibodies comprise at least one antigen binding domain. In one embodiment, the anti-PAI-1 antibodies comprise a VH having the amino acid sequence of SEQ ID NO. 6.

In another embodiment, the anti-PAI-1 antibodies comprise a VL having the amino acid sequence of SEQ ID NO. 8.

In yet another embodiment, the anti-PAI-1 antibodies comprise a VH having the amino acid sequence of SEQ ID NO. 6 and a VL having the amino acid sequence of SEQ ID NO. 8. See Table 16 and the Sequence Listing for a representation of VH and VL sequences encompassed in the present disclosure which can be present in any combination to form a present anti-PAI-1 antibody. In certain embodiments, an anti-PAI-1 antibody of the disclosure comprises a combination of any of the VH and VL sequences according to Table 16 and the Sequence Listing, wherein the antibody inhibits binding of human PAI-1 to tPA, but does not inhibit binding of human PAI-1 to vitronectin.

In one embodiment, the VH is selected from any of the VH represented in SEQ ID NOs: 6, 10, 14, 18, 22, 26, 30, 34, 38, 42, 46, 50, 54, 58, 62, 66, 70, 74, 78, 82, 86, 90, 94, 98, 102, 106, and 110).

In another embodiment, the VL is selected from any of the VL represented in SEQ ID NOs: 8, 12, 16, 20, 24, 28, 32, 36, 40, 44, 48, 52, 56, 60, 64, 68, 72, 76, 80, 84, 88, 92, 96, 100, 104, 108, and 112). In certain embodiments, the anti-PAI-1 antibodies are human or chimeric antibodies or antibody fragments. In certain embodiments, the anti-PAI-1 antibodies are human antibodies or antibody fragments. In certain embodiments, the anti-PAI-1 antibodies are human or chimeric antibodies or antibody fragments that immunospecifically bind to human PAI-1 and inhibit PAI-1 activity. In certain embodiments, the anti-PAI-1 antibodies are human or chimeric antibodies or antibody fragments that immunospecifically bind to human PAI-1 and inhibit binding of human PAI-1 to tPA, but do not inhibit binding of human PAI-1 to vitronectin.

In certain embodiments, the purified anti-PAI-1 antibodies comprise a VH and/or VL that has a given percent identify to at least one of the VH and/or VL sequences disclosed in Table 16 and the Sequence Listing. As used herein, the term “percent (%) sequence identity”, also including “homology” is defined as the percentage of amino acid residues or nucleotides in a candidate sequence that are identical with the amino acid residues or nucleotides in the reference sequences, such as parent antibody sequence, after aligning the sequences and introducing gaps, if necessary, to achieve the maximum percent sequence identity, and not considering any conservative substitutions as part of the sequence identity. Optimal alignment of the sequences for comparison may be produced, besides manually, by means of the local homology algorithm of Smith and Waterman, 1981, Ads App. Math. 2, 482, by means of the local homology algorithm of Neddleman and Wunsch, 1970, J. MoI. Biol. 48, 443, by means of the similarity search method of Pearson and Lipman, 1988, Proc. Natl. Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

In a specific embodiment, the anti-PAI-1 antibodies comprise a VH amino acid sequence having at least 65%, 70%, 75%, 80%, 85%, 90%, 95% or having at least 100% identity to the amino acid sequence of SEQ ID NO: 6.

In one embodiment, the anti-PAI-1 antibodies have a VH amino acid sequence having at least, 90%, 91%, 92%, 93%, 94%, 95%, 96%, 97%, 98%, 99% or having at least 100% identity to the amino acid sequence of SEQ ID NO: 6.

In a further embodiment, the anti-PAI-1 antibodies comprising a VH amino acid sequence with a given percent identify to SEQ ID NO: 6 have at least two or more (at least 3, 4, 5, 6, 7, 8, 9, 10, 11, 12, etc.) of the Desired Characteristics:

In certain embodiments, the antibody or antibody fragment has at least any number of these characteristics, such as at least 3, 4, 5, 6, 7, 8, 9, 10, 11, 12, 13, 14, 15, 16. Of course, such antibodies may optionally have additional characteristics. It should be understood that antibodies defined based on possessing any one or more of the foregoing properties possesses, at least, such one or more properties but may also possess other functional or structural characteristics.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3, 4) of the following characteristics:

inhibits binding of human PAI-1 to uPA and tPA;

preferentially binds to active human PAI-1 over latent human PAI-1;

can reduce the level of VCAM-1 in dermal tissues;

can reduce the level of TNF-alpha in dermal tissues;

can stimulate plasmin-mediated activation of MMP-1.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3) of the following characteristics:

affinity (K_{D}) between 5 pm to 200 pM for active human PAI-1, as assessed by surface plasmon resonance;

immunospecifically binds to a human PAI-1:vitronectin complex, but does not disrupt the binding of human PAI-1 to vitronectin;

specifically binds to the reactive center loop of PAI-1 that interacts with tPA and uPA.

In certain embodiments, the antibody or antibody fragment has an affinity (K_{D}) of between about 5 pM and 125 pM for active human PAI-1, as assessed by surface plasmon resonance. In certain embodiments, the antibody or antibody fragment has an affinity (K_{D}) of less than or equal to 100 pM for active human PAI-1, as assessed by surface plasmon resonance. In certain embodiments, the antibody or antibody fragment has an affinity (K_{D}) of less than or equal to 50 pM for active human PAI-1, as assessed by surface plasmon resonance.

In certain embodiments, the anti-PAI-1 antibodies comprise a heavy chain variable domain having an amino acid sequence identical to or comprising 1, 2, 3, 4, 5, 6, 7, 8, 9, or 10 amino acid residue substitutions relative to SEQ ID NO: 6. In certain embodiments, the substitutions are conservative amino acid substitutions.

In a specific embodiment, the anti-PAI-1 antibodies comprise a VL amino acid sequence having at least 65%, 70%, 75%, 80%, 85%, 90%, 95% or having at least 100% identity to the amino acid sequence of SEQ ID NO: 8.

In one embodiment, the anti-PAI-1 antibodies have a VL amino acid sequence having at least, 90%, 91%, 92%, 93%, 94%, 95%, 96%, 97%, 98%, 99% or having at least 100% identity to the amino acid sequence of SEQ ID NO: 8.

In a further embodiment, the anti-PAI-1 antibodies comprising a VL amino acid sequence with a given percent identify to SEQ ID NO: 8 have at least two or more (at least 3, 4, 5, 6, 7, 8, 9, 10, 11, 12, etc.) of the Desired Characteristics.

In certain embodiments, the antibody or antibody fragment has at least any number of these characteristics, such as at least 3, 4, 5, 6, 7, 8, 9, 10, 11, 12, 13, 14, 15, 16. Of course, such antibodies may optionally have additional characteristics. It should be understood that antibodies defined based on possessing any one or more of the foregoing properties possesses, at least, such one or more properties but may also possess other functional or structural characteristics.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3, 4) of the following characteristics:

inhibits binding of human PAI-1 to uPA and tPA;

preferentially binds to active human PAI-1 over latent human PAI-1;

can reduce the level of VCAM-1 in dermal tissues;

can reduce the level of TNF-alpha in dermal tissues;

can stimulate plasmin-mediated activation of MMP-1.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3) of the following characteristics:

affinity (K_{D}) between 5 pm to 200 pM for active human PAI-1, as assessed by surface plasmon resonance;

immunospecifically binds to a human PAI-1:vitronectin complex, but does not disrupt the binding of human PAI-1 to vitronectin;

specifically binds to the reactive center loop of PAI-1 that interacts with tPA and uPA.

In certain embodiments, the antibody or antibody fragment has an affinity (K_{D}) of between about 5 pM and 125 pM for active human PAI-1, as assessed by surface plasmon resonance. In certain embodiments, the antibody or antibody fragment has an affinity (K_{D}) of less than or equal to 100 pM for active human PAI-1, as assessed by surface plasmon resonance. In certain embodiments, the antibody or antibody fragment has an affinity (K_{D}) of less than or equal to 50 pM for active human PAI-1, as assessed by surface plasmon resonance.

In certain embodiments, the anti-PAI-1 antibodies comprise a light chain variable domain having an amino acid sequence identical to or comprising 1, 2, 3, 4, 5, 6, 7, 8, 9, or 10 amino acid residue substitutions relative to SEQ ID NO: 8. In certain embodiments, the substitutions are conservative amino acid substitutions.

In a specific embodiment a purified antibody of the disclosure immunospecifically binds PAI-1 and comprises a heavy chain variable domain having at least 90% identity to the amino acid of SEQ ID NO:6 and comprises a light chain variable domain having at least 90% identity to the amino acid sequence of SEQ ID NO:8, wherein said antibody immunospecifically binds to human PAI-1 and has any two or more of the foregoing functional properties. In certain embodiments, the anti-PAI-1 antibodies comprise a heavy chain variable domain having an amino acid sequence identical to or comprising 1, 2, 3, 4, 5, 6, 7, 8, 9, or 10 amino acid residue substitutions relative to SEQ ID NO: 2. In certain embodiments, the anti-PAI-1 antibodies comprise a light chain variable domain having an amino acid sequence identical to or comprising 1, 2, 3, 4, 5, 6, 7, 8, 9, or 10 amino acid residue substitutions relative to SEQ ID NO: 4. In certain embodiments, the substitutions are conservative amino acid substitutions.

In a specific embodiment, the anti-PAI-1 antibodies comprise a VH amino acid sequence having at least 65%, 70%, 75%, 80%, 85%, 90%, 95% or having at least 100% identity to the amino acid sequence of SEQ ID NO: 6. In one embodiment, the anti-PAI-1 antibodies have a VH amino acid sequence having at least, 90%, 91%, 92%, 93%, 94%, 95%, 96%, 97%, 98%, 99% or having at least 100% identity to the amino acid sequence of SEQ ID NO: 6. In a further embodiment, the anti-PAI-1 antibodies comprising a VH amino acid sequence with a given percent identify to SEQ ID NO: 6 have at least two or more (at least 3, 4, 5, 6, 7, 8, 9, 10, 11, 12, etc.) of the Desired Characteristics.

In certain embodiments, the antibody or antibody fragment has at least any number of these characteristics, such as at least 3, 4, 5, 6, 7, 8, 9, 10, 11, 12, 13, 14, 15, 16. Of course, such antibodies may optionally have additional characteristics. It should be understood that antibodies defined based on possessing any one or more of the foregoing properties possesses, at least, such one or more properties but may also possess other functional or structural characteristics.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3, 4) of the following characteristics:

inhibits binding of human PAI-1 to uPA and tPA;

preferentially binds to active human PAI-1 over latent human PAI-1;

can reduce the level of VCAM-1 in dermal tissues;

can reduce the level of TNF-alpha in dermal tissues;

can stimulate plasmin-mediated activation of MMP-1.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3) of the following characteristics:

affinity (K_{D}) between 5 pm to 200 pM for active human PAI-1, as assessed by surface plasmon resonance;

immunospecifically binds to a human PAI-1:vitronectin complex, but does not disrupt the binding of human PAI-1 to vitronectin;

specifically binds to the reactive center loop of PAI-1 that interacts with tPA and uPA.

In certain embodiments, the antibody or antibody fragment has an affinity (K_{D}) of between about 5 pM and 125 pM for active human PAI-1, as assessed by surface plasmon resonance. In certain embodiments, the antibody or antibody fragment has an affinity (K_{D}) of less than or equal to 100 pM for active human PAI-1, as assessed by surface plasmon resonance. In certain embodiments, the antibody or antibody fragment has an affinity (K_{D}) of less than or equal to 50 pM for active human PAI-1, as assessed by surface plasmon resonance.

In certain embodiments, the anti-PAI-1 antibodies comprise a heavy chain variable domain having an amino acid sequence identical to or comprising 1, 2, 3, 4, 5, 6, 7, 8, 9, or 10 amino acid residue substitutions relative to SEQ ID NO: 6. In certain embodiments, the substitutions are conservative amino acid substitutions.

In a specific embodiment, the anti-PAI-1 antibodies comprise a VL amino acid sequence having at least 65%, 70%, 75%, 80%, 85%, 90%, 95% or having at least 100% identity to the amino acid sequence of SEQ ID NO: 8.

In one embodiment, the anti-PAI-1 antibodies have a VL amino acid sequence having at least, 90%, 91%, 92%, 93%, 94%, 95%, 96%, 97%, 98%, 99% or having at least 100% identity to the amino acid sequence of SEQ ID NO: 8.

In a further embodiment, the anti-PAI-1 antibodies comprising a VL amino acid sequence with a given percent identify to SEQ ID NO: 8 have at least two or more (at least 3, 4, 5, 6, 7, 8, 9, 10, 11, 12, etc.) of theDesired Characteristics.

In certain embodiments, the antibody or antibody fragment has at least any number of these characteristics, such as at least 3, 4, 5, 6, 7, 8, 9, 10, 11, 12, 13, 14, 15, 16. Of course, such antibodies may optionally have additional characteristics. It should be understood that antibodies defined based on possessing any one or more of the foregoing properties possesses, at least, such one or more properties but may also possess other functional or structural characteristics.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3, 4) of the following characteristics:

inhibits binding of human PAI-1 to uPA and tPA;

preferentially binds to active human PAI-1 over latent human PAI-1;

can reduce the level of VCAM-1 in dermal tissues;

can reduce the level of TNF-alpha in dermal tissues;

can stimulate plasmin-mediated activation of MMP-1.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3) of the following characteristics:

affinity (K_{D}) between 5 pm to 200 pM for active human PAI-1, as assessed by surface plasmon resonance;

immunospecifically binds to a human PAI-1:vitronectin complex, but does not disrupt the binding of human PAI-1 to vitronectin;

specifically binds to the reactive center loop of PAI-1 that interacts with tPA and uPA.

In certain embodiments, the antibody or antibody fragment has an affinity (K_{D}) of between about 5 pM and 125 pM for active human PAI-1, as assessed by surface plasmon resonance. In certain embodiments, the antibody or antibody fragment has an affinity (K_{D}) of less than or equal to 100 pM for active human PAI-1, as assessed by surface plasmon resonance. In certain embodiments, the antibody or antibody fragment has an affinity (K_{D}) of less than or equal to 50 pM for active human PAI-1, as assessed by surface plasmon resonance.

In certain embodiments, the anti-PAI-1 antibodies comprise a light chain variable domain having an amino acid sequence identical to or comprising 1, 2, 3, 4, 5, 6, 7, 8, 9, or 10 amino acid residue substitutions relative to SEQ ID NO: 8. In certain embodiments, the substitutions are conservative amino acid substitutions.

In a specific embodiment a purified antibody of the disclosure immunospecifically binds PAI-1 and comprises a heavy chain variable domain having at least 90% identity to the amino acid of SEQ ID NO:6 and comprises a light chain variable domain having at least 90% identity to the amino acid sequence of SEQ ID NO:8, wherein said antibody immunospecifically binds to human PAI-1 and has any two or more of the foregoing functional properties.

In certain embodiments, the anti-PAI-1 antibodies comprise a heavy chain variable domain having an amino acid sequence identical to or comprising 1, 2, 3, 4, 5, 6, 7, 8, 9, or 10 amino acid residue substitutions relative to SEQ ID NO: 6.

In certain embodiments, the anti-PAI-1 antibodies comprise a light chain variable domain having an amino acid sequence identical to or comprising 1, 2, 3, 4, 5, 6, 7, 8, 9, or 10 amino acid residue substitutions relative to SEQ ID NO: 8. In certain embodiments, the substitutions are conservative amino acid substitutions.

In a specific embodiment, the anti-PAI-1 antibodies comprise a VH amino acid sequence having at least 65%, 70%, 75%, 80%, 85%, 90%, 95% or having at least 100% identity to the amino acid sequence of SEQ ID NO: 10.

In one embodiment, the anti-PAI-1 antibodies have a VH amino acid sequence having at least, 90%, 91%, 92%, 93%, 94%, 95%, 96%, 97%, 98%, 99% or having at least 100% identity to the amino acid sequence of SEQ ID NO: 10.

In a further embodiment, the anti-PAI-1 antibodies comprising a VH amino acid sequence with a given percent identify to SEQ ID NO: 10 have at least two or more (at least 3, 4, 5, 6, 7, 8, 9, 10, 11, 12, etc.) of the Desired Characteristics

In certain embodiments, the antibody or antibody fragment has at least any number of these characteristics, such as at least 3, 4, 5, 6, 7, 8, 9, 10, 11, 12, 13, 14, 15, 16. Of course, such antibodies may optionally have additional characteristics. It should be understood that antibodies defined based on possessing any one or more of the foregoing properties possesses, at least, such one or more properties but may also possess other functional or structural characteristics.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3, 4) of the following characteristics:

inhibits binding of human PAI-1 to uPA and tPA;

preferentially binds to active human PAI-1 over latent human PAI-1;

can reduce the level of VCAM-1 in dermal tissues;

can reduce the level of TNF-alpha in dermal tissues;

can stimulate plasmin-mediated activation of MMP-1.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3) of the following characteristics:

affinity (K_{D}) between 5 pm to 200 pM for active human PAI-1, as assessed by surface plasmon resonance;

immunospecifically binds to a human PAI-1:vitronectin complex, but does not disrupt the binding of human PAI-1 to vitronectin;

specifically binds to the reactive center loop of PAI-1 that interacts with tPA and uPA.

In certain embodiments, the antibody or antibody fragment has an affinity (K_{D}) of between about 5 pM and 125 pM for active human PAI-1, as assessed by surface plasmon resonance. In certain embodiments, the antibody or antibody fragment has an affinity (K_{D}) of less than or equal to 100 pM for active human PAI-1, as assessed by surface plasmon resonance. In certain embodiments, the antibody or antibody fragment has an affinity (K_{D}) of less than or equal to 50 pM for active human PAI-1, as assessed by surface plasmon resonance.

In certain embodiments, the anti-PAI-1 antibodies comprise a heavy chain variable domain having an amino acid sequence identical to or comprising 1, 2, 3, 4, 5, 6, 7, 8, 9, or 10 amino acid residue substitutions relative to SEQ ID NO: 10. In certain embodiments, the substitutions are conservative amino acid substitutions.

In a specific embodiment, the anti-PAI-1 antibodies comprise a VL amino acid sequence having at least 65%, 70%, 75%, 80%, 85%, 90%, 95% or having at least 100% identity to the amino acid sequence of SEQ ID NO: 12.

In one embodiment, the anti-PAI-1 antibodies have a VL amino acid sequence having at least, 90%, 91%, 92%, 93%, 94%, 95%, 96%, 97%, 98%, 99% or having at least 100% identity to the amino acid sequence of SEQ ID NO: 12.

In a further embodiment, the anti-PAI-1 antibodies comprising a VL amino acid sequence with a given percent identify to SEQ ID NO: 12 have at least two or more (at least 3, 4, 5, 6, 7, 8, 9, 10, 11, 12, etc.) of the Desired Characteristics.

In certain embodiments, the antibody or antibody fragment has at least any number of these characteristics, such as at least 3, 4, 5, 6, 7, 8, 9, 10, 11, 12, 13, 14, 15, 16. Of course, such antibodies may optionally have additional characteristics. It should be understood that antibodies defined based on possessing any one or more of the foregoing properties possesses, at least, such one or more properties but may also possess other functional or structural characteristics.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3, 4) of the following characteristics:

inhibits binding of human PAI-1 to uPA and tPA;

preferentially binds to active human PAI-1 over latent human PAI-1;

can reduce the level of VCAM-1 in dermal tissues;

can reduce the level of TNF-alpha in dermal tissues;

can stimulate plasmin-mediated activation of MMP-1.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3) of the following characteristics:

affinity (K_{D}) between 5 pm to 200 pM for active human PAI-1, as assessed by surface plasmon resonance;

immunospecifically binds to a human PAI-1:vitronectin complex, but does not disrupt the binding of human PAI-1 to vitronectin;

specifically binds to the reactive center loop of PAI-1 that interacts with tPA and uPA.

In certain embodiments, the antibody or antibody fragment has an affinity (K_{D}) of between about 5 pM and 125 pM for active human PAI-1, as assessed by surface plasmon resonance. In certain embodiments, the antibody or antibody fragment has an affinity (K_{D}) of less than or equal to 100 pM for active human PAI-1, as assessed by surface plasmon resonance. In certain embodiments, the antibody or antibody fragment has an affinity (K_{D}) of less than or equal to 50 pM for active human PAI-1, as assessed by surface plasmon resonance.

In certain embodiments, the anti-PAI-1 antibodies comprise a light chain variable domain having an amino acid sequence identical to or comprising 1, 2, 3, 4, 5, 6, 7, 8, 9, or 10 amino acid residue substitutions relative to SEQ ID NO: 12. In certain embodiments, the substitutions are conservative amino acid substitutions.

In a specific embodiment a purified antibody of the disclosure immunospecifically binds PAI-1 and comprises a heavy chain variable domain having at least 90% identity to the amino acid of SEQ ID NO: 10 and comprises a light chain variable domain having at least 90% identity to the amino acid sequence of SEQ ID NO: 12, wherein said antibody immunospecifically binds to human PAI-1 and has any two or more of the foregoing functional properties.

In certain embodiments, the anti-PAI-1 antibodies comprise a heavy chain variable domain having an amino acid sequence identical to or comprising 1, 2, 3, 4, 5, 6, 7, 8, 9, or 10 amino acid residue substitutions relative to SEQ ID NO: 10.

In certain embodiments, the anti-PAI-1 antibodies comprise a light chain variable domain having an amino acid sequence identical to or comprising 1, 2, 3, 4, 5, 6, 7, 8, 9, or 10 amino acid residue substitutions relative to SEQ ID NO: 12. In certain embodiments, the substitutions are conservative amino acid substitutions.

In a specific embodiment, the anti-PAI-1 antibodies comprise a VH amino acid sequence having at least 65%, 70%, 75%, 80%, 85%, 90%, 95% or having at least 100% identity to the amino acid sequence of SEQ ID NO: 34.

In one embodiment, the anti-PAI-1 antibodies have a VH amino acid sequence having at least, 90%, 91%, 92%, 93%, 94%, 95%, 96%, 97%, 98%, 99% or having at least 100% identity to the amino acid sequence of SEQ ID NO: 34.

In a further embodiment, the anti-PAI-1 antibodies comprising a VH amino acid sequence with a given percent identify to SEQ ID NO: 34 have at least two or more (at least 3, 4, 5, 6, 7, 8, 9, 10, 11, 12, etc.) of the Desired Characteristics.

In certain embodiments, the antibody or antibody fragment has at least any number of these characteristics, such as at least 3, 4, 5, 6, 7, 8, 9, 10, 11, 12, 13, 14, 15, 16. Of course, such antibodies may optionally have additional characteristics. It should be understood that antibodies defined based on possessing any one or more of the foregoing properties possesses, at least, such one or more properties but may also possess other functional or structural characteristics.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3, 4) of the following characteristics:

inhibits binding of human PAI-1 to uPA and tPA;

preferentially binds to active human PAI-1 over latent human PAI-1;

can reduce the level of VCAM-1 in dermal tissues;

can reduce the level of TNF-alpha in dermal tissues;

can stimulate plasmin-mediated activation of MMP-1.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3) of the following characteristics:

affinity (K_{D}) between 5 pm to 200 pM for active human PAI-1, as assessed by surface plasmon resonance;

immunospecifically binds to a human PAI-1:vitronectin complex, but does not disrupt the binding of human PAI-1 to vitronectin;

specifically binds to the reactive center loop of PAI-1 that interacts with tPA and uPA.

In certain embodiments, the antibody or antibody fragment has an affinity (K_{D}) of between about 5 pM and 125 pM for active human PAI-1, as assessed by surface plasmon resonance. In certain embodiments, the antibody or antibody fragment has an affinity (K_{D}) of less than or equal to 100 pM for active human PAI-1, as assessed by surface plasmon resonance. In certain embodiments, the antibody or antibody fragment has an affinity (K_{D}) of less than or equal to 50 pM for active human PAI-1, as assessed by surface plasmon resonance.

In certain embodiments, the anti-PAI-1 antibodies comprise a heavy chain variable domain having an amino acid sequence identical to or comprising 1, 2, 3, 4, 5, 6, 7, 8, 9, or 10 amino acid residue substitutions relative to SEQ ID NO: 34. In certain embodiments, the substitutions are conservative amino acid substitutions.

In a specific embodiment, the anti-PAI-1 antibodies comprise a VL amino acid sequence having at least 65%, 70%, 75%, 80%, 85%, 90%, 95% or having at least 100% identity to the amino acid sequence of SEQ ID NO: 36.

In one embodiment, the anti-PAI-1 antibodies have a VL amino acid sequence having at least, 90%, 91%, 92%, 93%, 94%, 95%, 96%, 97%, 98%, 99% or having at least 100% identity to the amino acid sequence of SEQ ID NO: 36.

In a further embodiment, the anti-PAI-1 antibodies comprising a VL amino acid sequence with a given percent identify to SEQ ID NO: 36 have at least two or more (at least 3, 4, 5, 6, 7, 8, 9, 10, 11, 12, etc.) of the Desired Characteristics.

In certain embodiments, the antibody or antibody fragment has at least any number of these characteristics, such as at least 3, 4, 5, 6, 7, 8, 9, 10, 11, 12, 13, 14, 15, 16. Of course, such antibodies may optionally have additional characteristics. It should be understood that antibodies defined based on possessing any one or more of the foregoing properties possesses, at least, such one or more properties but may also possess other functional or structural characteristics.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3, 4) of the following characteristics:

inhibits binding of human PAI-1 to uPA and tPA;

preferentially binds to active human PAI-1 over latent human PAI-1;

can reduce the level of VCAM-1 in dermal tissues;

can reduce the level of TNF-alpha in dermal tissues;

can stimulate plasmin-mediated activation of MMP-1.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3) of the following characteristics:

affinity (K_{D}) between 5 pm to 200 pM for active human PAI-1, as assessed by surface plasmon resonance;

immunospecifically binds to a human PAI-1:vitronectin complex, but does not disrupt the binding of human PAI-1 to vitronectin;

specifically binds to the reactive center loop of PAI-1 that interacts with tPA and uPA.

In certain embodiments, the antibody or antibody fragment has an affinity (K_{D}) of between about 5 pM and 125 pM for active human PAI-1, as assessed by surface plasmon resonance. In certain embodiments, the antibody or antibody fragment has an affinity (K_{D}) of less than or equal to 100 pM for active human PAI-1, as assessed by surface plasmon resonance. In certain embodiments, the antibody or antibody fragment has an affinity (K_{D}) of less than or equal to 50 pM for active human PAI-1, as assessed by surface plasmon resonance.

In certain embodiments, the anti-PAI-1 antibodies comprise a light chain variable domain having an amino acid sequence identical to or comprising 1, 2, 3, 4, 5, 6, 7, 8, 9, or 10 amino acid residue substitutions relative to SEQ ID NO: 36. In certain embodiments, the substitutions are conservative amino acid substitutions.

In a specific embodiment a purified antibody of the disclosure immunospecifically binds PAI-1 and comprises a heavy chain variable domain having at least 90% identity to the amino acid of SEQ ID NO: 34 and comprises a light chain variable domain having at least 90% identity to the amino acid sequence of SEQ ID NO: 36, wherein said antibody immunospecifically binds to human PAI-1 and has any two or more of the foregoing functional properties.

In certain embodiments, the anti-PAI-1 antibodies comprise a heavy chain variable domain having an amino acid sequence identical to or comprising 1, 2, 3, 4, 5, 6, 7, 8, 9, or 10 amino acid residue substitutions relative to SEQ ID NO: 34. In certain embodiments, the anti-PAI-1 antibodies comprise a light chain variable domain having an amino acid sequence identical to or comprising 1, 2, 3, 4, 5, 6, 7, 8, 9, or 10 amino acid residue substitutions relative to SEQ ID NO: 36. In certain embodiments, the substitutions are conservative amino acid substitutions.

In a specific embodiment, the anti-PAI-1 antibodies comprise a VH amino acid sequence having at least 65%, 70%, 75%, 80%, 85%, 90%, 95% or having at least 100% identity to the amino acid sequence of SEQ ID NO: 66.

In one embodiment, the anti-PAI-1 antibodies have a VH amino acid sequence having at least, 90%, 91%, 92%, 93%, 94%, 95%, 96%, 97%, 98%, 99% or having at least 100% identity to the amino acid sequence of SEQ ID NO: 66. In a further embodiment, the anti-PAI-1 antibodies comprising a VH amino acid sequence with a given percent identify to SEQ ID NO: 66 have at least two or more (at least 3, 4, 5, 6, 7, 8, 9, 10, 11, 12, etc.) of the Desired Characteristics.

In certain embodiments, the antibody or antibody fragment has at least any number of these characteristics, such as at least 3, 4, 5, 6, 7, 8, 9, 10, 11, 12, 13, 14, 15, 16. Of course, such antibodies may optionally have additional characteristics. It should be understood that antibodies defined based on possessing any one or more of the foregoing properties possesses, at least, such one or more properties but may also possess other functional or structural characteristics.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3, 4) of the following characteristics:

inhibits binding of human PAI-1 to uPA and tPA;

preferentially binds to active human PAI-1 over latent human PAI-1;

can reduce the level of VCAM-1 in dermal tissues;

can reduce the level of TNF-alpha in dermal tissues;

can stimulate plasmin-mediated activation of MMP-1.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3) of the following characteristics:

affinity (K_{D}) between 5 pm to 200 pM for active human PAI-1, as assessed by surface plasmon resonance;

immunospecifically binds to a human PAI-1:vitronectin complex, but does not disrupt the binding of human PAI-1 to vitronectin;

specifically binds to the reactive center loop of PAI-1 that interacts with tPA and uPA.

In certain embodiments, the antibody or antibody fragment has an affinity (K_{D}) of between about 5 pM and 125 pM for active human PAI-1, as assessed by surface plasmon resonance. In certain embodiments, the antibody or antibody fragment has an affinity (K_{D}) of less than or equal to 100 pM for active human PAI-1, as assessed by surface plasmon resonance. In certain embodiments, the antibody or antibody fragment has an affinity (K_{D}) of less than or equal to 50 pM for active human PAI-1, as assessed by surface plasmon resonance.

In certain embodiments, the anti-PAI-1 antibodies comprise a heavy chain variable domain having an amino acid sequence identical to or comprising 1, 2, 3, 4, 5, 6, 7, 8, 9, or 10 amino acid residue substitutions relative to SEQ ID NO: 66. In certain embodiments, the substitutions are conservative amino acid substitutions.

In a specific embodiment, the anti-PAI-1 antibodies comprise a VL amino acid sequence having at least 65%, 70%, 75%, 80%, 85%, 90%, 95% or having at least 100% identity to the amino acid sequence of SEQ ID NO: 68.

In one embodiment, the anti-PAI-1 antibodies have a VL amino acid sequence having at least, 90%, 91%, 92%, 93%, 94%, 95%, 96%, 97%, 98%, 99% or having at least 100% identity to the amino acid sequence of SEQ ID NO: 68.

In a further embodiment, the anti-PAI-1 antibodies comprising a VL amino acid sequence with a given percent identify to SEQ ID NO: 68 have at least two or more (at least 3, 4, 5, 6, 7, 8, 9, 10, 11, 12, etc.) of the Desired Characteristics.

In certain embodiments, the antibody or antibody fragment has at least any number of these characteristics, such as at least 3, 4, 5, 6, 7, 8, 9, 10, 11, 12, 13, 14, 15, 16. Of course, such antibodies may optionally have additional characteristics. It should be understood that antibodies defined based on possessing any one or more of the foregoing properties possesses, at least, such one or more properties but may also possess other functional or structural characteristics.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3, 4) of the following characteristics:

inhibits binding of human PAI-1 to uPA and tPA;

preferentially binds to active human PAI-1 over latent human PAI-1;

can reduce the level of VCAM-1 in dermal tissues;

can reduce the level of TNF-alpha in dermal tissues;

can stimulate plasmin-mediated activation of MMP-1.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3) of the following characteristics:

affinity (K_{D}) between 5 pm to 200 pM for active human PAI-1, as assessed by surface plasmon resonance;

immunospecifically binds to a human PAI-1:vitronectin complex, but does not disrupt the binding of human PAI-1 to vitronectin;

specifically binds to the reactive center loop of PAI-1 that interacts with tPA and uPA.

In certain embodiments, the antibody or antibody fragment has an affinity (K_{D}) of between about 5 pM and 125 pM for active human PAI-1, as assessed by surface plasmon resonance. In certain embodiments, the antibody or antibody fragment has an affinity (K_{D}) of less than or equal to 100 pM for active human PAI-1, as assessed by surface plasmon resonance. In certain embodiments, the antibody or antibody fragment has an affinity (K_{D}) of less than or equal to 50 pM for active human PAI-1, as assessed by surface plasmon resonance.

In certain embodiments, the anti-PAI-1 antibodies comprise a light chain variable domain having an amino acid sequence identical to or comprising 1, 2, 3, 4, 5, 6, 7, 8, 9, or 10 amino acid residue substitutions relative to SEQ ID NO: 68. In certain embodiments, the substitutions are conservative amino acid substitutions.

In a specific embodiment a purified antibody of the disclosure immunospecifically binds PAI-1 and comprises a heavy chain variable domain having at least 90% identity to the amino acid of SEQ ID NO: 66 and comprises a light chain variable domain having at least 90% identity to the amino acid sequence of SEQ ID NO: 68, wherein said antibody immunospecifically binds to human PAI-1 and has any two or more of the foregoing functional properties.

In certain embodiments, the anti-PAI-1 antibodies comprise a heavy chain variable domain having an amino acid sequence identical to or comprising 1, 2, 3, 4, 5, 6, 7, 8, 9, or 10 amino acid residue substitutions relative to SEQ ID NO: 66.

In certain embodiments, the anti-PAI-1 antibodies comprise a light chain variable domain having an amino acid sequence identical to or comprising 1, 2, 3, 4, 5, 6, 7, 8, 9, or 10 amino acid residue substitutions relative to SEQ ID NO: 68. In certain embodiments, the substitutions are conservative amino acid substitutions.

In a specific embodiment, the anti-PAI-1 antibodies comprise a VH amino acid sequence having at least 65%, 70%, 75%, 80%, 85%, 90%, 95% or having at least 100% identity to the amino acid sequence of SEQ ID NO: 82.

In one embodiment, the anti-PAI-1 antibodies have a VH amino acid sequence having at least, 90%, 91%, 92%, 93%, 94%, 95%, 96%, 97%, 98%, 99% or having at least 100% identity to the amino acid sequence of SEQ ID NO: 82.

In a further embodiment, the anti-PAI-1 antibodies comprising a VH amino acid sequence with a given percent identify to SEQ ID NO: 82 have at least two or more (at least 3, 4, 5, 6, 7, 8, 9, 10, 11, 12, etc.) of the Desired Characteristics

In certain embodiments, the antibody or antibody fragment has at least any number of these characteristics, such as at least 3, 4, 5, 6, 7, 8, 9, 10, 11, 12, 13, 14, 15, 16. Of course, such antibodies may optionally have additional characteristics. It should be understood that antibodies defined based on possessing any one or more of the foregoing properties possesses, at least, such one or more properties but may also possess other functional or structural characteristics.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3, 4) of the following characteristics:

inhibits binding of human PAI-1 to uPA and tPA;

preferentially binds to active human PAI-1 over latent human PAI-1;

can reduce the level of VCAM-1 in dermal tissues;

can reduce the level of TNF-alpha in dermal tissues;

can stimulate plasmin-mediated activation of MMP-1.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3) of the following characteristics:

affinity (K_{D}) between 5 pm to 200 pM for active human PAI-1, as assessed by surface plasmon resonance;

immunospecifically binds to a human PAI-1:vitronectin complex, but does not disrupt the binding of human PAI-1 to vitronectin;

specifically binds to the reactive center loop of PAI-1 that interacts with tPA and uPA.

In certain embodiments, the antibody or antibody fragment has an affinity (K_{D}) of between about 5 pM and 125 pM for active human PAI-1, as assessed by surface plasmon resonance. In certain embodiments, the antibody or antibody fragment has an affinity (K_{D}) of less than or equal to 100 pM for active human PAI-1, as assessed by surface plasmon resonance. In certain embodiments, the antibody or antibody fragment has an affinity (K_{D}) of less than or equal to 50 pM for active human PAI-1, as assessed by surface plasmon resonance.

In certain embodiments, the anti-PAI-1 antibodies comprise a heavy chain variable domain having an amino acid sequence identical to or comprising 1, 2, 3, 4, 5, 6, 7, 8, 9, or 10 amino acid residue substitutions relative to SEQ ID NO: 82. In certain embodiments, the substitutions are conservative amino acid substitutions.

In a specific embodiment, the anti-PAI-1 antibodies comprise a VL amino acid sequence having at least 65%, 70%, 75%, 80%, 85%, 90%, 95% or having at least 100% identity to the amino acid sequence of SEQ ID NO: 84.

In one embodiment, the anti-PAI-1 antibodies have a VL amino acid sequence having at least, 90%, 91%, 92%, 93%, 94%, 95%, 96%, 97%, 98%, 99% or having at least 100% identity to the amino acid sequence of SEQ ID NO: 84.

In a further embodiment, the anti-PAI-1 antibodies comprising a VL amino acid sequence with a given percent identify to SEQ ID NO: 84 have at least two or more (at least 3, 4, 5, 6, 7, 8, 9, 10, 11, 12, etc.) of the Desired Characteristics.

In certain embodiments, the antibody or antibody fragment has at least any number of these characteristics, such as at least 3, 4, 5, 6, 7, 8, 9, 10, 11, 12, 13, 14, 15, 16. Of course, such antibodies may optionally have additional characteristics. It should be understood that antibodies defined based on possessing any one or more of the foregoing properties possesses, at least, such one or more properties but may also possess other functional or structural characteristics.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3, 4) of the following characteristics:

inhibits binding of human PAI-1 to uPA and tPA;

preferentially binds to active human PAI-1 over latent human PAI-1;

can reduce the level of VCAM-1 in dermal tissues;

can reduce the level of TNF-alpha in dermal tissues;

can stimulate plasmin-mediated activation of MMP-1.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3) of the following characteristics:

affinity (K_{D}) between 5 pm to 200 pM for active human PAI-1, as assessed by surface plasmon resonance;

immunospecifically binds to a human PAI-1:vitronectin complex, but does not disrupt the binding of human PAI-1 to vitronectin;

specifically binds to the reactive center loop of PAI-1 that interacts with tPA and uPA.

In certain embodiments, the antibody or antibody fragment has an affinity (K_{D}) of between about 5 pM and 125 pM for active human PAI-1, as assessed by surface plasmon resonance. In certain embodiments, the antibody or antibody fragment has an affinity (K_{D}) of less than or equal to 100 pM for active human PAI-1, as assessed by surface plasmon resonance. In certain embodiments, the antibody or antibody fragment has an affinity (K_{D}) of less than or equal to 50 pM for active human PAI-1, as assessed by surface plasmon resonance.

In certain embodiments, the anti-PAI-1 antibodies comprise a light chain variable domain having an amino acid sequence identical to or comprising 1, 2, 3, 4, 5, 6, 7, 8, 9, or 10 amino acid residue substitutions relative to SEQ ID NO: 84. In certain embodiments, the substitutions are conservative amino acid substitutions.

In a specific embodiment a purified antibody of the disclosure immunospecifically binds PAI-1 and comprises a heavy chain variable domain having at least 90% identity to the amino acid of SEQ ID NO: 82 and comprises a light chain variable domain having at least 90% identity to the amino acid sequence of SEQ ID NO: 84, wherein said antibody immunospecifically binds to human PAI-1 and has any two or more of the foregoing functional properties.

In certain embodiments, the anti-PAI-1 antibodies comprise a heavy chain variable domain having an amino acid sequence identical to or comprising 1, 2, 3, 4, 5, 6, 7, 8, 9, or 10 amino acid residue substitutions relative to SEQ ID NO: 82.

In certain embodiments, the anti-PAI-1 antibodies comprise a light chain variable domain having an amino acid sequence identical to or comprising 1, 2, 3, 4, 5, 6, 7, 8, 9, or 10 amino acid residue substitutions relative to SEQ ID NO: 84. In certain embodiments, the substitutions are conservative amino acid substitutions.

In a specific embodiment, the anti-PAI-1 antibodies comprise a VH amino acid sequence having at least 65%, 70%, 75%, 80%, 85%, 90%, 95% or having at least 100% identity to the amino acid sequence of SEQ ID NO: 110.

In one embodiment, the anti-PAI-1 antibodies have a VH amino acid sequence having at least, 90%, 91%, 92%, 93%, 94%, 95%, 96%, 97%, 98%, 99% or having at least 100% identity to the amino acid sequence of SEQ ID NO: 110.

In a further embodiment, the anti-PAI-1 antibodies comprising a VH amino acid sequence with a given percent identify to SEQ ID NO: 110 have at least two or more (at least 3, 4, 5, 6, 7, 8, 9, 10, 11, 12, etc.) of the Desired Characteristics.

In certain embodiments, the antibody or antibody fragment has at least any number of these characteristics, such as at least 3, 4, 5, 6, 7, 8, 9, 10, 11, 12, 13, 14, 15, 16. Of course, such antibodies may optionally have additional characteristics. It should be understood that antibodies defined based on possessing any one or more of the foregoing properties possesses, at least, such one or more properties but may also possess other functional or structural characteristics.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3, 4) of the following characteristics:

inhibits binding of human PAI-1 to uPA and tPA;

preferentially binds to active human PAI-1 over latent human PAI-1;

can reduce the level of VCAM-1 in dermal tissues;

can reduce the level of TNF-alpha in dermal tissues;

can stimulate plasmin-mediated activation of MMP-1.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3) of the following characteristics:

affinity (K_{D}) between 5 pm to 200 pM for active human PAI-1, as assessed by surface plasmon resonance;

immunospecifically binds to a human PAI-1:vitronectin complex, but does not disrupt the binding of human PAI-1 to vitronectin;

specifically binds to the reactive center loop of PAI-1 that interacts with tPA and uPA.

In certain embodiments, the antibody or antibody fragment has an affinity (K_{D}) of between about 5 pM and 125 pM for active human PAI-1, as assessed by surface plasmon resonance. In certain embodiments, the antibody or antibody fragment has an affinity (K_{D}) of less than or equal to 100 pM for active human PAI-1, as assessed by surface plasmon resonance. In certain embodiments, the antibody or antibody fragment has an affinity (K_{D}) of less than or equal to 50 pM for active human PAI-1, as assessed by surface plasmon resonance.

In certain embodiments, the anti-PAI-1 antibodies comprise a heavy chain variable domain having an amino acid sequence identical to or comprising 1, 2, 3, 4, 5, 6, 7, 8, 9, or 10 amino acid residue substitutions relative to SEQ ID NO: 110. In certain embodiments, the substitutions are conservative amino acid substitutions.

In a specific embodiment, the anti-PAI-1 antibodies comprise a VL amino acid sequence having at least 65%, 70%, 75%, 80%, 85%, 90%, 95% or having at least 100% identity to the amino acid sequence of SEQ ID NO: 112.

In one embodiment, the anti-PAI-1 antibodies have a VL amino acid sequence having at least, 90%, 91%, 92%, 93%, 94%, 95%, 96%, 97%, 98%, 99% or having at least 100% identity to the amino acid sequence of SEQ ID NO: 112.

In a further embodiment, the anti-PAI-1 antibodies comprising a VL amino acid sequence with a given percent identify to SEQ ID NO: 112 have at least two or more (at least 3, 4, 5, 6, 7, 8, 9, 10, 11, 12, etc.) of the Desired Characteristics.

In certain embodiments, the antibody or antibody fragment has at least any number of these characteristics, such as at least 3, 4, 5, 6, 7, 8, 9, 10, 11, 12, 13, 14, 15, 16. Of course, such antibodies may optionally have additional characteristics. It should be understood that antibodies defined based on possessing any one or more of the foregoing properties possesses, at least, such one or more properties but may also possess other functional or structural characteristics.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3, 4) of the following characteristics:

inhibits binding of human PAI-1 to uPA and tPA;

preferentially binds to active human PAI-1 over latent human PAI-1;

can reduce the level of VCAM-1 in dermal tissues;

can reduce the level of TNF-alpha in dermal tissues;

can stimulate plasmin-mediated activation of MMP-1.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3) of the following characteristics:

affinity (K_{D}) between 5 pm to 200 pM for active human PAI-1, as assessed by surface plasmon resonance;

immunospecifically binds to a human PAI-1:vitronectin complex, but does not disrupt the binding of human PAI-1 to vitronectin;

specifically binds to the reactive center loop of PAI-1 that interacts with tPA and uPA.

In certain embodiments, the antibody or antibody fragment has an affinity (K_{D}) of between about 5 pM and 125 pM for active human PAI-1, as assessed by surface plasmon resonance. In certain embodiments, the antibody or antibody fragment has an affinity (K_{D}) of less than or equal to 100 pM for active human PAI-1, as assessed by surface plasmon resonance. In certain embodiments, the antibody or antibody fragment has an affinity (K_{D}) of less than or equal to 50 pM for active human PAI-1, as assessed by surface plasmon resonance.

In certain embodiments, the anti-PAI-1 antibodies comprise a light chain variable domain having an amino acid sequence identical to or comprising 1, 2, 3, 4, 5, 6, 7, 8, 9, or 10 amino acid residue substitutions relative to SEQ ID NO: 110. In certain embodiments, the substitutions are conservative amino acid substitutions.

In a specific embodiment a purified antibody of the disclosure immunospecifically binds PAI-1 and comprises a heavy chain variable domain having at least 90% identity to the amino acid of SEQ ID NO: 110 and comprises a light chain variable domain having at least 90% identity to the amino acid sequence of SEQ ID NO: 112, wherein said antibody immunospecifically binds to human PAI-1 and has any two or more of the foregoing functional properties.

In certain embodiments, the anti-PAI-1 antibodies comprise a heavy chain variable domain having an amino acid sequence identical to or comprising 1, 2, 3, 4, 5, 6, 7, 8, 9, or 10 amino acid residue substitutions relative to SEQ ID NO: 110.

In certain embodiments, the anti-PAI-1 antibodies comprise a light chain variable domain having an amino acid sequence identical to or comprising 1, 2, 3, 4, 5, 6, 7, 8, 9, or 10 amino acid residue substitutions relative to SEQ ID NO: 112. In certain embodiments, the substitutions are conservative amino acid substitutions.

For any of the foregoing, the disclosure contemplates anti-PAI-1 antibodies comprising any combination of the foregoing VH and VL domains, as well as anti-PAI-1 antibodies having any combination of the functional properties described herein. Further, these VH and/or VL domains may be provided as antibody fragments or as part of a larger antibody, such as an antibody comprising an Fc region.

The foregoing description applies equally to antibodies and antibody fragments of the disclosure. Moreover, the disclosure contemplates antibodies and antibody fragments having any combination of the structural and/or functional features described herein. Further, the various VH and VL domains described above can be described with other features of the disclosure described herein. Any such anti-PAI-1 antibodies and antibody fragments can be used in any of the methods described herein.

Note that certain of the VH and VL domains provided in the Sequence Listing may correspond to the same amino acid sequence. They are provided separately to illustrate the experimental results, as well as the relationships among certain antibodies identified. However, when a sequence identifier is used to describe the sequence of a particular antibody, it should be understood, unless otherwise specified, to refer to the underlying sequence itself without requiring or assuming a source of that sequence.

2. Complementarity Determining Regions (CDRs)

While the variable domain (VH and VL) comprises the antigen-binding region; the variability is not evenly distributed through the variable domains of antibodies. It is concentrated in segments called Complementarity Determining Regions (CDRs), both in the light chain (VL or VK) and the heavy chain (VH) variable domains. The more highly conserved portions of the variable domains are called the framework regions (FR). The variable domains of native heavy and light chains each comprise four FR, largely adopting a β-sheet configuration, connected by three CDRs, which form loops connecting, and in some cases forming part of, the β-sheet structure. The CDRs in each chain are held together in close proximity by the FR and, with the CDRs from the other chain, contribute to the formation of the antigen-binding site of antibodies (see, Kabat et al., Supra). The three CDRs of the heavy chain are designated CDR-H1, CDR-H2, and CDR-H3, and the three CDRs of the light chain are designated CDR-L1, CDR-L2, and CDR-L3. The Kabat numbering system is used herein. As such, CDR-H1 begins at approximately amino acid 31 (i.e., approximately 9 residues after the first cysteine residue), includes approximately 5-7 amino acids, and ends at the next tyrosine residue. CDR-H2 begins at the fifteenth residue after the end of CDR-H1, includes approximately 16-19 amino acids, and ends at the next arginine or lysine residue. CDR-H3 begins at approximately the thirty third amino acid residue after the end of CDR-H2; includes 3-25 amino acids; and ends at the sequence W-G-X-G, where X is any amino acid. CDR-L1 begins at approximately residue 24 (i.e., following a cysteine residue); includes approximately 10-17 residues; and ends at the next tyrosine residue. CDR-L2 begins at approximately the sixteenth residue after the end of CDR-L1 and includes approximately 7 residues. CDR-L3 begins at approximately the thirty third residue after the end of CDR-L2; includes approximately 7-11 residues and ends at the sequence F-G-X-G, where X is any amino acid. Note that CDRs vary considerably from antibody to antibody (and by definition will not exhibit homology with the Kabat consensus sequences).

The present anti-PAI-1 antibodies comprise at least one antigen binding domain that comprises at least one complementarity determining region (CDR1, CDR2 and CDR3). In one embodiment, the anti-PAI-1 antibodies comprise a VH that comprises at least one VH CDR (e.g., CDR-H1, CDR-H2 or CDR-H3). In another embodiment, the anti-PAI-1 antibodies comprise a VL that comprises at least one VL CDR (e.g., CDR-L1, CDR-L2 or CDR-L3).

In certain embodiments, anti-PAI-1 antibodies of the disclosure for use in the claimed methods comprises a combination of any CDR-H1 sequence of Table 2, any CDR-H2 sequence of Table 2, any CDR-H3 sequence of Table 2, any CDR-L1 sequence of Table 2, any CDR-L2 sequence of Table 2 and any CDR-L3 sequence of Table 2, wherein the antibody specifically binds human PAI-1 and inhibits PAI-1 activity, such as inhibits binding of human PAI-1 to tPA. In certain embodiments, said antibody is an antibody fragment. In certain embodiments, said antibody is a human, humanized or chimeric antibody. In certain embodiments, such an antibody has at least two or more (at least 3, 4, 5, 6, 7, 8, 9, 10, 11, 12, etc.) of the Desired Characteristics.

In certain embodiments, the antibody or antibody fragment has at least any number of these characteristics, such as at least 3, 4, 5, 6, 7, 8, 9, 10, 11, 12, 13, 14, 15, 16. Of course, such antibodies may optionally have additional characteristics. It should be understood that antibodies defined based on possessing any one or more of the foregoing properties possesses, at least, such one or more properties but may also possess other functional or structural characteristics.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3, 4) of the following characteristics:

inhibits binding of human PAI-1 to uPA and tPA;

preferentially binds to active human PAI-1 over latent human PAI-1;

can reduce the level of VCAM-1 in dermal tissues;

can reduce the level of TNF-alpha in dermal tissues;

can stimulate plasmin-mediated activation of MMP-1.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3) of the following characteristics:

affinity (K_{D}) between 5 pm to 200 pM for active human PAI-1, as assessed by surface plasmon resonance;

immunospecifically binds to a human PAI-1:vitronectin complex, but does not disrupt the binding of human PAI-1 to vitronectin;

specifically binds to the reactive center loop of PAI-1 that interacts with tPA and uPA.

In certain embodiments, the antibody or antibody fragment has an affinity (K_{D}) of between about 5 pM and 125 pM for active human PAI-1, as assessed by surface plasmon resonance. In certain embodiments, the antibody or antibody fragment has an affinity (K_{D}) of less than or equal to 100 pM for active human PAI-1, as assessed by surface plasmon resonance. In certain embodiments, the antibody or antibody fragment has an affinity (K_{D}) of less than or equal to 50 pM for active human PAI-1, as assessed by surface plasmon resonance.

In certain embodiments, anti-PAI-1 antibodies comprise

(a) a VH CDR1 having an amino acid sequence identical to or comprising 1, 2, or 3 amino acid residue substitutions relative to SEQ ID NO: 113,
(b) a VH CDR2 having an amino acid sequence identical to or comprising 1, 2, or 3 amino acid residue substitutions relative to SEQ ID NO: 114 and/or
(c) a VH CDR3 having an amino acid sequence identical to or comprising 1, 2, or 3 amino acid residue substitutions relative to SEQ ID NO: 115.

In another embodiment, the anti-PAI-1 antibodies comprise

(a) a VL CDR1 having an amino acid sequence identical to or comprising 1, 2, or 3 amino acid residue substitutions relative to SEQ ID NO: 176,
(b) a VL CDR2 having an amino acid sequence identical to or comprising 1, 2, or 3 amino acid residue substitutions relative to SEQ ID NO: 117 and/or
(c) a VL CDR3 having an amino acid sequence identical to or comprising 1, 2, or 3 amino acid residue substitutions relative to SEQ ID NO: 118.

The disclosure contemplates antibodies and antibody fragments having any combination of the foregoing VH and VL CDRs. For example, antibodies comprising:

(a) a VH CDR1 having a sequence identical to SEQ ID NO: 113,
(b) a VH CDR2 having an amino acid sequence comprising one amino acid substitution relative to SEQ ID NO: 114, and
(c) a VH CDR3 having an amino acid sequence comprising one amino acid substitution relative to SEQ ID NO: 115.

By way of further example, antibodies comprising:

(a) a VL CDR1 having an amino acid sequence comprising one amino acid substitution relative to SEQ ID NO: 176,
(b) a VL CDR2 having an amino acid sequence identical to SEQ ID NO: 117, and
(c) a VL CDR3 having an amino acid sequence comprising three substitutions relative to SEQ ID NO: 118.

All other combinations are similarly contemplated. In certain embodiments, the antibodies or antibody fragments are human or chimeric antibodies that immunospecifically bind to human PAI-1 and inhibit binding of human PAI-1 to tPA, but do not inhibit the binding of human PAI-1 to vitronectin.

The foregoing description of the VH and VL domains is intended to refer to all possible combinations. Thus, for example, description of a VH CDR1 having an amino acid sequence identical to or comprising 1, 2, or 3 amino acid substitutions relative to SEQ ID NO: 113 refers to any of the following embodiments:

(a) a VH CDR1 having an amino acid sequence identical to SEQ ID NO: 113;
(b) a VH CDR1 having an amino acid sequence comprising 1 amino acid residue substitution relative to SEQ ID NO: 113;
(c) a VH CDR 1 having an amino acid sequence comprising 2 amino acid residue substitution relative to SEQ ID NO: 113;
(d) a VH CDR1 having an amino acid sequence comprising 3 amino acid residue substitutions relative to SEQ ID NO: 113; etc.

In certain embodiments, anti-PAI-1 antibodies comprise:

(a) a VH CDR1 having an amino acid sequence identical to or comprising 1, 2, or 3 amino acid residue substitutions relative to SEQ ID NO: 113,
(b) a VH CDR2 having an amino acid sequence identical to or comprising 1, 2, or 3 amino acid residue substitutions relative to SEQ ID NO: 114,
(c) a VH CDR3 having an amino acid sequence identical to or comprising 1, 2, or 3 amino acid residue substitutions relative to SEQ ID NO: 115,
(d) a VL CDR1 having an amino acid sequence identical to or comprising 1, 2, or 3 amino acid residue substitutions relative to SEQ ID NO: 176,
(e) a VL CDR2 having an amino acid sequence identical to or comprising 1, 2, or 3 amino acid residue substitutions relative to SEQ ID NO: 117 and
(f) a VL CDR3 having an amino acid sequence identical to or comprising 1, 2, or 3 amino acid residue substitutions relative to SEQ ID NO: 118.

The disclosure contemplates antibodies and antibody fragments having any combination of the foregoing VH and VL CDRs. For example, antibodies comprising:

(a) a VH CDR1 having a sequence identical to SEQ ID NO: 113,
(b) a VH CDR2 having an amino acid sequence comprising one amino acid substitution relative to SEQ ID NO: 114,
(c) a VH CDR3 having an amino acid sequence comprising one amino acid substitution relative to SEQ ID NO: 115.

By way of further example, antibodies comprising:

(a) a VL CDR1 having an amino acid sequence comprising one substitution relative to SEQ ID NO: 176,
(b) a VL CDR2 having an amino acid sequence identical to SEQ ID NO: 117, and
(c) a VL CDR3 having an amino acid sequence comprising three substitutions relative to SEQ ID NO: 118.

All other combinations are similarly contemplated. In certain embodiments, the antibodies or antibody fragments are human or chimeric antibodies that immunospecifically bind to human PAI-1 and inhibit PAI-1 activity, such as inhibiting binding of human PAI-1 to tPA, but not inhibiting the binding of human PAI-1 to vitronectin.

In a specific embodiment, an anti-PAI-1 antibody comprises:

(a) a VH CDR1 having the amino acid sequence of SEQ ID NO: 113;
(b) a VH CDR2 having the amino acid sequence of SEQ ID NO: 161;
(c) a VH CDR3 having the amino acid sequence of SEQ ID NO: 171;
(d) a VL CDR1 having the amino acid sequence of SEQ ID NO: 176;
(e) a VL CDR2 having the amino acid sequence of SEQ ID NO: 117; and
(f) a VL CDR3 having the amino acid sequence of SEQ ID NO: 186.

In a further embodiment, anti-PAI-1 antibodies of the disclosure that immunospecifically binds human PAI-1 and comprise:

(a) a VH CDR1 having an amino acid sequence identical to or comprising 1, 2, or 3 amino acid residue substitutions relative to SEQ ID NO: 187;
(b) a VH CDR2 having an amino acid sequence identical to or comprising 1, 2, or 3 amino acid residue substitutions relative to SEQ ID NO: 161;
(c) a VH CDR3 having an amino acid sequence identical to or comprising 1, 2, or 3 amino acid residue substitutions relative to SEQ ID NO: 171;
(d) a VL CDR1 having an amino acid sequence identical to or comprising 1, 2, or 3 amino acid residue substitutions relative to SEQ ID NO: 176;
(e) a VL CDR2 having an amino acid sequence identical to or comprising 1, 2, or 3 amino acid residue substitutions relative to SEQ ID NO: 117; and
(f) a VL CDR3 having an amino acid sequence identical to or comprising 1, 2, or 3 amino acid residue substitutions relative to SEQ ID NO: 186, and has at least two or more (at least 3, 4, 5, 6, 7, 8, 9, 10, 11, 12, etc.) of the Desired Characteristics.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3, 4) of the following characteristics:

inhibits binding of human PAI-1 to uPA and tPA;

preferentially binds to active human PAI-1 over latent human PAI-1;

can reduce the level of VCAM-1 in dermal tissues;

can reduce the level of TNF-alpha in dermal tissues;

can stimulate plasmin-mediated activation of MMP-1.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3) of the following characteristics:

affinity (K_{D}) between 5 pm to 200 pM for active human PAI-1, as assessed by surface plasmon resonance;

immunospecifically binds to a human PAI-1:vitronectin complex, but does not disrupt the binding of human PAI-1 to vitronectin;

specifically binds to the reactive center loop of PAI-1 that interacts with tPA and uPA.

In certain embodiments, the antibody or antibody fragment has an affinity (K_{D}) of between about 5 pM and 125 pM for active human PAI-1, as assessed by surface plasmon resonance. In certain embodiments, the antibody or antibody fragment has an affinity (K_{D}) of less than or equal to 100 pM for active human PAI-1, as assessed by surface plasmon resonance. In certain embodiments, the antibody or antibody fragment has an affinity (K_{D}) of less than or equal to 50 pM for active human PAI-1, as assessed by surface plasmon resonance.

It is well known in the art that VH CDR3 and VL CDR3 domains play an important role in the binding specificity/affinity of an antibody for an antigen (Xu and Davis, Immunity, 13: 37-45, 2000).

Accordingly, in one embodiment, anti-PAI-1 antibodies comprise a VH CDR3 having an amino acid sequence identical to or comprising 1, 2, or 3 amino acid residue substitutions relative to SEQ ID NO: 115.

In another embodiment, the anti-PAI-1 antibodies comprise a VL CDR3 having an amino acid sequence identical to or comprising 1, 2, or 3 amino acid residue substitutions relative to SEQ ID NO: 118.

In yet another embodiment, the anti-PAI-1 antibodies comprise a VH CDR3 having an amino acid sequence identical to or comprising 1, 2, or 3 amino acid residue substitutions relative to SEQ ID NO: 115 and a VL CDR3 having an amino acid sequence identical to or comprising 1, 2, or 3 amino acid residue substitutions relative to SEQ ID NO: 118. The remaining portions of the anti-PAI-1 antibodies (e.g. CDR1, CDR2, VH, VL, etc.) may comprise specific sequences disclosed herein or known sequences provided the anti-PAI-1 antibodies immunospecifically bind to human PAI-1 and having two or more of the Desired Characteristics.

In certain embodiments, anti-PAI-1 antibodies comprise:

(a) a VH CDR1 having an amino acid sequence identical to or comprising 1, 2, or 3 amino acid residue substitutions relative to SEQ ID NO: 113,
(b) a VH CDR2 having an amino acid sequence identical to or comprising 1, 2, or 3 amino acid residue substitutions relative to SEQ ID NO: 160 and
(c) a VH CDR3 having an amino acid sequence identical to or comprising 1, 2, or 3 amino acid residue substitutions relative to SEQ ID NO: 115.

In another embodiment, anti-PAI-1 antibodies comprise

(a) a VL CDR1 having an amino acid sequence identical to or comprising 1, 2, or 3 amino acid residue substitutions relative to SEQ ID NO: 172,
(b) a VL CDR2 having an amino acid sequence identical to or comprising 1, 2, or 3 amino acid residue substitutions relative to SEQ ID NO: 117 and
(c) a VL CDR3 having an amino acid sequence identical to or comprising 1, 2, or 3 amino acid residue substitutions relative to SEQ ID NO: 118.

The disclosure contemplates antibodies and antibody fragments having any combination of the foregoing VH and VL CDRs. For example, antibodies comprising:

(a) a VH CDR1 having a sequence identical to SEQ ID NO: 113,
(b) a VH CDR2 having an amino acid sequence comprising one amino acid substitution relative to SEQ ID NO: 160,
(c) a VH CDR3 having an amino acid sequence comprising one amino acid substitution relative to SEQ ID NO: 115.

By way of further example, antibodies comprising:

(a) a VL CDR1 having an amino acid sequence comprising one amino acid substitution relative to SEQ ID NO: 172,
(b) a VL CDR2 having an amino acid sequence identical to SEQ ID NO: 117, and
(c) a VL CDR3 having an amino acid sequence comprising three substitutions relative to SEQ ID NO: 118.

All other combinations are similarly contemplated. In certain embodiments, the antibodies or antibody fragments are human or chimeric antibodies that immunospecifically bind to human PAI-1 and inhibit PAI-1 activity, such as binding of human PAI-1 to tPA, but do not inhibit the binding of human PAI-1 to vitronectin. The foregoing description of the VH and VL domains is intended to refer to all possible combinations.

In certain embodiments, anti-PAI-1 antibodies comprise

(a) a VH CDR1 having an amino acid sequence identical to or comprising 1, 2, or 3 amino acid residue substitutions relative to SEQ ID NO: 113,
(b) a VH CDR2 having an amino acid sequence identical to or comprising 1, 2, or 3 amino acid residue substitutions relative to SEQ ID NO: 160,
(c) a VH CDR3 having an amino acid sequence identical to or comprising 1, 2, or 3 amino acid residue substitutions relative to SEQ ID NO: 115,
(d) a VL CDR1 having an amino acid sequence identical to or comprising 1, 2, or 3 amino acid residue substitutions relative to SEQ ID NO: 172,
(e) a VL CDR2 having an amino acid sequence identical to or comprising 1, 2, or 3 amino acid residue substitutions relative to SEQ ID NO: 117 and
(f) a VL CDR3 having an amino acid sequence identical to or comprising 1, 2, or 3 amino acid residue substitutions relative to SEQ ID NO: 118.

The disclosure contemplates antibodies and antibody fragments having any combination of the foregoing VH and VL CDRs. For example, antibodies comprising:

(a) a VH CDR1 having a sequence identical to SEQ ID NO: 113,
(b) a VH CDR2 having an amino acid sequence comprising one amino acid substitution relative to SEQ ID NO: 160,
(c) a VH CDR3 having an amino acid sequence comprising one amino acid substitution relative to SEQ ID NO: 115.

By way of further example, antibodies comprising:

(a) a VL CDR1 having an amino acid sequence comprising one substitution relative to SEQ ID NO: 172,
(c) a VL CDR2 having an amino acid sequence identical to SEQ ID NO: 117, and a VL CDR3 having an amino acid sequence comprising three substitutions relative to SEQ ID NO: 118.

All other combinations are similarly contemplated. In certain embodiments, the antibodies or antibody fragments are human or chimeric antibodies that immunospecifically bind to human PAI-1 and inhibit PAI-1 activity, such as binding of human PAI-1 to tPA, but do not inhibit the binding of human PAI-1 to vitronectin. The foregoing description of the VH and VL domains is intended to refer to all possible combinations.

In a specific embodiment, anti-PAI-1 antibody comprise

(a) a VH CDR1 having the amino acid sequence of SEQ ID NO: 113;
(b) a VH CDR2 having the amino acid sequence of SEQ ID NO: 160;
(c) a VH CDR3 having the amino acid sequence of SEQ ID NO: 115;
(d) a VL CDR1 having the amino acid sequence of SEQ ID NO: 172;
(e) a VL CDR2 having the amino acid sequence of SEQ ID NO: 117; and
(f) a VL CDR3 having the amino acid sequence of SEQ ID NO: 118.

In a further embodiment, antibodies of the disclosure that immunospecifically binds human PAI-1 and comprise:

(a) a VH CDR1 having an amino acid sequence identical to or comprising 1, 2, or 3 amino acid residue substitutions relative to SEQ ID NO: 113;
(b) a VH CDR2 having an amino acid sequence identical to or comprising 1, 2, or 3 amino acid residue substitutions relative to SEQ ID NO: 160;
(c) a VH CDR3 having an amino acid sequence identical to or comprising 1, 2, or 3 amino acid residue substitutions relative to SEQ ID NO: 115;
(d) a VL CDR1 having an amino acid sequence identical to or comprising 1, 2, or 3 amino acid residue substitutions relative to SEQ ID NO: 172;
(e) a VL CDR2 having an amino acid sequence identical to or comprising 1, 2, or 3 amino acid residue substitutions relative to SEQ ID NO: 117; and
(f) a VL CDR3 having an amino acid sequence identical to or comprising 1, 2, or 3 amino acid residue substitutions relative to SEQ ID NO: 186, and has least two or more (at least 3, 4, 5, 6, 7, 8, 9, 10, 11, 12, etc.) of the Desired Characteristics.

In another embodiment, anti-PAI-1 antibodies comprise a VH CDR3 having an amino acid sequence identical to or comprising 1, 2, or 3 amino acid residue substitutions relative to SEQ ID NO: 171.

In another embodiment, the anti-PAI-1 antibodies comprise a VL CDR3 having an amino acid sequence identical to or comprising 1, 2, or 3 amino acid residue substitutions relative to SEQ ID NO: 185.

In yet another embodiment, the anti-PAI-1 antibodies comprise a VH CDR3 having an amino acid sequence identical to or comprising 1, 2, or 3 amino acid residue substitutions relative to SEQ ID NO: 171 and a VL CDR3 having an amino acid sequence identical to or comprising 1, 2, or 3 amino acid residue substitutions relative to SEQ ID NO: 185. The remaining portions of the anti-PAI-1 antibodies (e.g. CDR1, CDR2, VH, VL, etc.) may comprise specific sequences disclosed herein or known sequences provided the anti-PAI-1 antibodies immunospecifically bind to human PAI-1 and having two or more of the Desired Characteristics.

In certain embodiments, anti-PAI-1 antibodies comprise:

(a) a VH CDR1 having an amino acid sequence identical to or comprising 1, 2, or 3 amino acid residue substitutions relative to SEQ ID NO: 113,
(b) a VH CDR2 having an amino acid sequence identical to or comprising 1, 2, or 3 amino acid residue substitutions relative to SEQ ID NO: 161 and
(c) a VH CDR3 having an amino acid sequence identical to or comprising 1, 2, or 3 amino acid residue substitutions relative to SEQ ID NO: 171.

In another embodiment, anti-PAI-1 antibodies comprise:

(a) a VL CDR1 having an amino acid sequence identical to or comprising 1, 2, or 3 amino acid residue substitutions relative to SEQ ID NO: 173,
(b) a VL CDR2 having an amino acid sequence identical to or comprising 1, 2, or 3 amino acid residue substitutions relative to SEQ ID NO: 117 and
(c) a VL CDR3 having an amino acid sequence identical to or comprising 1, 2, or 3 amino acid residue substitutions relative to SEQ ID NO: 185.

The disclosure contemplates antibodies and antibody fragments having any combination of the foregoing VH and VL CDRs. For example, antibodies comprising:

(a) a VH CDR1 having a sequence identical to SEQ ID NO: 113,
(b) a VH CDR2 having an amino acid sequence comprising one amino acid substitution relative to SEQ ID NO: 161,
(c) a VH CDR3 having an amino acid sequence comprising one amino acid substitution relative to SEQ ID NO: 171.

By way of further example, antibodies comprising:

(a) a VL CDR1 having an amino acid sequence comprising one amino acid substitution relative to SEQ ID NO: 173,
(b) a VL CDR2 having an amino acid sequence identical to SEQ ID NO: 117, and
(c) a VL CDR3 having an amino acid sequence comprising three substitutions relative to SEQ ID NO: 185.

All other combinations are similarly contemplated. In certain embodiments, the antibodies or antibody fragments are human or chimeric antibodies that immunospecifically bind to human PAI-1 and inhibit PAI-1 activity, such as binding of human PAI-1 to tPA, but do not inhibit the binding of human PAI-1 to vitronectin. The foregoing description of the VH and VL domains is intended to refer to all possible combinations.

In certain embodiments, anti-PAI-1 antibodies comprise:

(a) a VH CDR1 having an amino acid sequence identical to or comprising 1, 2, or 3 amino acid residue substitutions relative to SEQ ID NO: 113,
(b) a VH CDR2 having an amino acid sequence identical to or comprising 1, 2, or 3 amino acid residue substitutions relative to SEQ ID NO: 161,
(c) a VH CDR3 having an amino acid sequence identical to or comprising 1, 2, or 3 amino acid residue substitutions relative to SEQ ID NO: 171,
(d) a VL CDR1 having an amino acid sequence identical to or comprising 1, 2, or 3 amino acid residue substitutions relative to SEQ ID NO: 173,
(e) a VL CDR2 having an amino acid sequence identical to or comprising 1, 2, or 3 amino acid residue substitutions relative to SEQ ID NO: 117 and
(f) a VL CDR3 having an amino acid sequence identical to or comprising 1, 2, or 3 amino acid residue substitutions relative to SEQ ID NO: 185.

The disclosure contemplates antibodies and antibody fragments having any combination of the foregoing VH and VL CDRs. For example, antibodies comprising:

(a) a VH CDR1 having a sequence identical to SEQ ID NO: 113,
(b) a VH CDR2 having an amino acid sequence comprising one amino acid substitution relative to SEQ ID NO: 161,
(c) a VH CDR3 having an amino acid sequence comprising one amino acid substitution relative to SEQ ID NO: 171.

By way of further example, antibodies comprising:

(a) a VL CDR1 having an amino acid sequence comprising one substitution relative to SEQ ID NO: 173,
(c) a VL CDR2 having an amino acid sequence identical to SEQ ID NO: 117, and a VL CDR3 having an amino acid sequence comprising three substitutions relative to SEQ ID NO: 185.

All other combinations are similarly contemplated. In certain embodiments, the antibodies or antibody fragments are human or chimeric antibodies that immunospecifically bind to human PAI-1 and inhibit PAI-1 activity, such as binding of human PAI-1 to tPA, but do not inhibit the binding of human PAI-1 to vitronectin. The foregoing description of the VH and VL domains is intended to refer to all possible combinations.

In a specific embodiment, anti-PAI-1 antibody comprise:

(a) a VH CDR1 having the amino acid sequence of SEQ ID NO: 113;
(b) a VH CDR2 having the amino acid sequence of SEQ ID NO: 161;
(c) a VH CDR3 having the amino acid sequence of SEQ ID NO: 171;
(d) a VL CDR1 having the amino acid sequence of SEQ ID NO: 173;
(e) a VL CDR2 having the amino acid sequence of SEQ ID NO: 117; and
(f) a VL CDR3 having the amino acid sequence of SEQ ID NO: 185.

In a further embodiment, antibodies of the disclosure that immunospecifically binds human PAI-1 and comprise:

(a) a VH CDR1 having an amino acid sequence identical to or comprising 1, 2, or 3 amino acid residue substitutions relative to SEQ ID NO: 113;
(b) a VH CDR2 having an amino acid sequence identical to or comprising 1, 2, or 3 amino acid residue substitutions relative to SEQ ID NO: 161;
(c) a VH CDR3 having an amino acid sequence identical to or comprising 1, 2, or 3 amino acid residue substitutions relative to SEQ ID NO: 171;
(d) a VL CDR1 having an amino acid sequence identical to or comprising 1, 2, or 3 amino acid residue substitutions relative to SEQ ID NO: 173;
(e) a VL CDR2 having an amino acid sequence identical to or comprising 1, 2, or 3 amino acid residue substitutions relative to SEQ ID NO: 117; and
(f) a VL CDR3 having an amino acid sequence identical to or comprising 1, 2, or 3 amino acid residue substitutions relative to SEQ ID NO: 185, and has least two or more (at least 3, 4, 5, 6, 7, 8, 9, 10, 11, 12, etc.) of the Desired Characteristics.

In certain embodiments, anti-PAI-1 antibodies comprise

(a) a VH CDR1 having an amino acid sequence identical to or comprising 1, 2, or 3 amino acid residue substitutions relative to SEQ ID NO: 187,
(b) a VH CDR2 having an amino acid sequence identical to or comprising 1, 2, or 3 amino acid residue substitutions relative to SEQ ID NO: 161 and
(c) a VH CDR3 having an amino acid sequence identical to or comprising 1, 2, or 3 amino acid residue substitutions relative to SEQ ID NO: 171.

In another embodiment, anti-PAI-1 antibodies comprise:

(a) a VL CDR1 having an amino acid sequence identical to or comprising 1, 2, or 3 amino acid residue substitutions relative to SEQ ID NO: 176,
(b) a VL CDR2 having an amino acid sequence identical to or comprising 1, 2, or 3 amino acid residue substitutions relative to SEQ ID NO: 117 and
(c) a VL CDR3 having an amino acid sequence identical to or comprising 1, 2, or 3 amino acid residue substitutions relative to SEQ ID NO: 185.

The disclosure contemplates antibodies and antibody fragments having any combination of the foregoing VH and VL CDRs. For example, antibodies comprising:

(a) a VH CDR1 having a sequence identical to SEQ ID NO: 187,
(b) a VH CDR2 having an amino acid sequence comprising one amino acid substitution relative to SEQ ID NO: 161,
(c) a VH CDR3 having an amino acid sequence comprising one amino acid substitution relative to SEQ ID NO: 171.

By way of further example, antibodies comprising:

(a) a VL CDR1 having an amino acid sequence comprising one amino acid substitution relative to SEQ ID NO: 176,
(b) a VL CDR2 having an amino acid sequence identical to SEQ ID NO: 117, and
(c) a VL CDR3 having an amino acid sequence comprising three substitutions relative to SEQ ID NO: 185.

All other combinations are similarly contemplated. In certain embodiments, the antibodies or antibody fragments are human or chimeric antibodies that immunospecifically bind to human PAI-1 and inhibit PAI-1 activity, such as binding of human PAI-1 to tPA, but do not inhibit the binding of human PAI-1 to vitronectin. The foregoing description of the VH and VL domains is intended to refer to all possible combinations.

In certain embodiments, anti-PAI-1 antibodies comprise:

(a) a VH CDR1 having an amino acid sequence identical to or comprising 1, 2, or 3 amino acid residue substitutions relative to SEQ ID NO: 187,
(b) a VH CDR2 having an amino acid sequence identical to or comprising 1, 2, or 3 amino acid residue substitutions relative to SEQ ID NO: 161,
(c) a VH CDR3 having an amino acid sequence identical to or comprising 1, 2, or 3 amino acid residue substitutions relative to SEQ ID NO: 171,
(d) a VL CDR1 having an amino acid sequence identical to or comprising 1, 2, or 3 amino acid residue substitutions relative to SEQ ID NO: 176,
(e) a VL CDR2 having an amino acid sequence identical to or comprising 1, 2, or 3 amino acid residue substitutions relative to SEQ ID NO: 117 and
(f) a VL CDR3 having an amino acid sequence identical to or comprising 1, 2, or 3 amino acid residue substitutions relative to SEQ ID NO: 185.

The disclosure contemplates antibodies and antibody fragments having any combination of the foregoing VH and VL CDRs. For example, antibodies comprising:

(a) a VH CDR1 having a sequence identical to SEQ ID NO: 187,
(b) a VH CDR2 having an amino acid sequence comprising one amino acid substitution relative to SEQ ID NO: 161,
(c) a VH CDR3 having an amino acid sequence comprising one amino acid substitution relative to SEQ ID NO: 171.

By way of further example, antibodies comprising:

(a) a VL CDR1 having an amino acid sequence comprising one substitution relative to SEQ ID NO: 176,
(c) a VL CDR2 having an amino acid sequence identical to SEQ ID NO: 117, and a VL CDR3 having an amino acid sequence comprising three substitutions relative to SEQ ID NO: 185.

All other combinations are similarly contemplated. In certain embodiments, the antibodies or antibody fragments are human or chimeric antibodies that immunospecifically bind to human PAI-1 and inhibit PAI-1 activity, such as binding of human PAI-1 to tPA, but do not inhibit the binding of human PAI-1 to vitronectin. The foregoing description of the VH and VL domains is intended to refer to all possible combinations.

In a specific embodiment, anti-PAI-1 antibody comprise:

(a) a VH CDR1 having the amino acid sequence of SEQ ID NO: 187;
(b) a VH CDR2 having the amino acid sequence of SEQ ID NO: 161;
(c) a VH CDR3 having the amino acid sequence of SEQ ID NO: 171;
(d) a VL CDR1 having the amino acid sequence of SEQ ID NO: 176;
(e) a VL CDR2 having the amino acid sequence of SEQ ID NO: 117; and
(f) a VL CDR3 having the amino acid sequence of SEQ ID NO: 185.

In a further embodiment, antibodies of the disclosure that immunospecifically binds human PAI-1 and comprise:

(a) a VH CDR1 having an amino acid sequence identical to or comprising 1, 2, or 3 amino acid residue substitutions relative to SEQ ID NO: 187;
(b) a VH CDR2 having an amino acid sequence identical to or comprising 1, 2, or 3 amino acid residue substitutions relative to SEQ ID NO: 161;
(c) a VH CDR3 having an amino acid sequence identical to or comprising 1, 2, or 3 amino acid residue substitutions relative to SEQ ID NO: 171;
(d) a VL CDR1 having an amino acid sequence identical to or comprising 1, 2, or 3 amino acid residue substitutions relative to SEQ ID NO: 176;
(e) a VL CDR2 having an amino acid sequence identical to or comprising 1, 2, or 3 amino acid residue substitutions relative to SEQ ID NO: 117; and
(f) a VL CDR3 having an amino acid sequence identical to or comprising 1, 2, or 3 amino acid residue substitutions relative to SEQ ID NO: 185, and has least two or more (at least 3, 4, 5, 6, 7, 8, 9, 10, 11, 12, etc.) of the Desired Characteristics.

In certain embodiments, anti-PAI-1 antibodies comprise

(a) a VH CDR1 having an amino acid sequence identical to or comprising 1, 2, or 3 amino acid residue substitutions relative to SEQ ID NO: 187,
(b) a VH CDR2 having an amino acid sequence identical to or comprising 1, 2, or 3 amino acid residue substitutions relative to SEQ ID NO: 160 and
(c) a VH CDR3 having an amino acid sequence identical to or comprising 1, 2, or 3 amino acid residue substitutions relative to SEQ ID NO: 171.

In another embodiment, anti-PAI-1 antibodies comprise:

(a) a VL CDR1 having an amino acid sequence identical to or comprising 1, 2, or 3 amino acid residue substitutions relative to SEQ ID NO: 179,
(b) a VL CDR2 having an amino acid sequence identical to or comprising 1, 2, or 3 amino acid residue substitutions relative to SEQ ID NO: 117 and
(c) a VL CDR3 having an amino acid sequence identical to or comprising 1, 2, or 3 amino acid residue substitutions relative to SEQ ID NO: 186.

The disclosure contemplates antibodies and antibody fragments having any combination of the foregoing VH and VL CDRs. For example, antibodies comprising:

(a) a VH CDR1 having a sequence identical to SEQ ID NO: 187,
(b) a VH CDR2 having an amino acid sequence comprising one amino acid substitution relative to SEQ ID NO: 160,
(c) a VH CDR3 having an amino acid sequence comprising one amino acid substitution relative to SEQ ID NO: 171.

By way of further example, antibodies comprising:

(a) a VL CDR1 having an amino acid sequence comprising one amino acid substitution relative to SEQ ID NO: 179,
(b) a VL CDR2 having an amino acid sequence identical to SEQ ID NO: 117, and
(c) a VL CDR3 having an amino acid sequence comprising three substitutions relative to SEQ ID NO: 186.

All other combinations are similarly contemplated. In certain embodiments, the antibodies or antibody fragments are human or chimeric antibodies that immunospecifically bind to human PAI-1 and inhibit PAI-1 activity, such as binding of human PAI-1 to tPA, but do not inhibit the binding of human PAI-1 to vitronectin. The foregoing description of the VH and VL domains is intended to refer to all possible combinations.

In certain embodiments, anti-PAI-1 antibodies comprise:

(a) a VH CDR1 having an amino acid sequence identical to or comprising 1, 2, or 3 amino acid residue substitutions relative to SEQ ID NO: 187,
(b) a VH CDR2 having an amino acid sequence identical to or comprising 1, 2, or 3 amino acid residue substitutions relative to SEQ ID NO: 160,
(c) a VH CDR3 having an amino acid sequence identical to or comprising 1, 2, or 3 amino acid residue substitutions relative to SEQ ID NO: 171,
(d) a VL CDR1 having an amino acid sequence identical to or comprising 1, 2, or 3 amino acid residue substitutions relative to SEQ ID NO: 179,
(e) a VL CDR2 having an amino acid sequence identical to or comprising 1, 2, or 3 amino acid residue substitutions relative to SEQ ID NO: 117 and
(f) a VL CDR3 having an amino acid sequence identical to or comprising 1, 2, or 3 amino acid residue substitutions relative to SEQ ID NO: 186.

The disclosure contemplates antibodies and antibody fragments having any combination of the foregoing VH and VL CDRs. For example, antibodies comprising:

(a) a VH CDR1 having a sequence identical to SEQ ID NO: 187,
(b) a VH CDR2 having an amino acid sequence comprising one amino acid substitution relative to SEQ ID NO: 160,
(c) a VH CDR3 having an amino acid sequence comprising one amino acid substitution relative to SEQ ID NO: 171.

By way of further example, antibodies comprising:

(a) a VL CDR1 having an amino acid sequence comprising one substitution relative to SEQ ID NO: 179,
(c) a VL CDR2 having an amino acid sequence identical to SEQ ID NO: 117, and a VL CDR3 having an amino acid sequence comprising three substitutions relative to SEQ ID NO: 186.

All other combinations are similarly contemplated. In certain embodiments, the antibodies or antibody fragments are human or chimeric antibodies that immunospecifically bind to human PAI-1 and inhibit PAI-1 activity, such as binding of human PAI-1 to tPA, but do not inhibit the binding of human PAI-1 to vitronectin. The foregoing description of the VH and VL domains is intended to refer to all possible combinations.

In a specific embodiment, anti-PAI-1 antibody comprise:

(a) a VH CDR1 having the amino acid sequence of SEQ ID NO: 187;
(b) a VH CDR2 having the amino acid sequence of SEQ ID NO: 160;
(c) a VH CDR3 having the amino acid sequence of SEQ ID NO: 171;
(d) a VL CDR1 having the amino acid sequence of SEQ ID NO: 179;
(e) a VL CDR2 having the amino acid sequence of SEQ ID NO: 117; and
(f) a VL CDR3 having the amino acid sequence of SEQ ID NO: 186.

In a further embodiment, antibodies of the disclosure that immunospecifically binds human PAI-1 and comprise

(a) a VH CDR1 having an amino acid sequence identical to or comprising 1, 2, or 3 amino acid residue substitutions relative to SEQ ID NO: 187;
(b) a VH CDR2 having an amino acid sequence identical to or comprising 1, 2, or 3 amino acid residue substitutions relative to SEQ ID NO: 160;
(c) a VH CDR3 having an amino acid sequence identical to or comprising 1, 2, or 3 amino acid residue substitutions relative to SEQ ID NO: 171;
(d) a VL CDR1 having an amino acid sequence identical to or comprising 1, 2, or 3 amino acid residue substitutions relative to SEQ ID NO: 179;
(e) a VL CDR2 having an amino acid sequence identical to or comprising 1, 2, or 3 amino acid residue substitutions relative to SEQ ID NO: 117; and
(f) a VL CDR3 having an amino acid sequence identical to or comprising 1, 2, or 3 amino acid residue substitutions relative to SEQ ID NO: 186, and has least two or more (at least 3, 4, 5, 6, 7, 8, 9, 10, 11, 12, etc.) of the Desired Characteristics.

In certain embodiments, anti-PAI-1 antibodies comprise:

(a) a VH CDR1 having an amino acid sequence identical to or comprising 1, 2, or 3 amino acid residue substitutions relative to SEQ ID NO: 187,
(b) a VH CDR2 having an amino acid sequence identical to or comprising 1, 2, or 3 amino acid residue substitutions relative to SEQ ID NO: 161 and
(c) a VH CDR3 having an amino acid sequence identical to or comprising 1, 2, or 3 amino acid residue substitutions relative to SEQ ID NO: 171.

In another embodiment, anti-PAI-1 antibodies comprise:

(a) a VL CDR1 having an amino acid sequence identical to or comprising 1, 2, or 3 amino acid residue substitutions relative to SEQ ID NO: 175,
(b) a VL CDR2 having an amino acid sequence identical to or comprising 1, 2, or 3 amino acid residue substitutions relative to SEQ ID NO: 184 and
(c) a VL CDR3 having an amino acid sequence identical to or comprising 1, 2, or 3 amino acid residue substitutions relative to SEQ ID NO: 185.

The disclosure contemplates antibodies and antibody fragments having any combination of the foregoing VH and VL CDRs. For example, antibodies comprising:

(a) a VH CDR1 having a sequence identical to SEQ ID NO: 187,
(b) a VH CDR2 having an amino acid sequence comprising one amino acid substitution relative to SEQ ID NO: 161,
(c) a VH CDR3 having an amino acid sequence comprising one amino acid substitution relative to SEQ ID NO: 171.

By way of further example, antibodies comprising:

(a) a VL CDR1 having an amino acid sequence comprising one amino acid substitution relative to SEQ ID NO: 175,
(b) a VL CDR2 having an amino acid sequence identical to SEQ ID NO: 184, and
(c) a VL CDR3 having an amino acid sequence comprising three substitutions relative to SEQ ID NO: 185.

All other combinations are similarly contemplated. In certain embodiments, the antibodies or antibody fragments are human or chimeric antibodies that immunospecifically bind to human PAI-1 and inhibit PAI-1 activity, such as binding of human PAI-1 to tPA, but do not inhibit the binding of human PAI-1 to vitronectin. The foregoing description of the VH and VL domains is intended to refer to all possible combinations.

In certain embodiments, anti-PAI-1 antibodies comprise:

(a) a VH CDR1 having an amino acid sequence identical to or comprising 1, 2, or 3 amino acid residue substitutions relative to SEQ ID NO: 187,
(b) a VH CDR2 having an amino acid sequence identical to or comprising 1, 2, or 3 amino acid residue substitutions relative to SEQ ID NO: 161,
(c) a VH CDR3 having an amino acid sequence identical to or comprising 1, 2, or 3 amino acid residue substitutions relative to SEQ ID NO: 171,
(d) a VL CDR1 having an amino acid sequence identical to or comprising 1, 2, or 3 amino acid residue substitutions relative to SEQ ID NO: 175,
(e) a VL CDR2 having an amino acid sequence identical to or comprising 1, 2, or 3 amino acid residue substitutions relative to SEQ ID NO: 184 and
(f) a VL CDR3 having an amino acid sequence identical to or comprising 1, 2, or 3 amino acid residue substitutions relative to SEQ ID NO: 185.

The disclosure contemplates antibodies and antibody fragments having any combination of the foregoing VH and VL CDRs. For example, antibodies comprising:

(a) a VH CDR1 having a sequence identical to SEQ ID NO: 187,
(b) a VH CDR2 having an amino acid sequence comprising one amino acid substitution relative to SEQ ID NO: 161,
(c) a VH CDR3 having an amino acid sequence comprising one amino acid substitution relative to SEQ ID NO: 171.

By way of further example, antibodies comprising:

(a) a VL CDR1 having an amino acid sequence comprising one substitution relative to SEQ ID NO: 175,
(c) a VL CDR2 having an amino acid sequence identical to SEQ ID NO: 184, and a VL CDR3 having an amino acid sequence comprising three substitutions relative to SEQ ID NO: 185. All other combinations are similarly contemplated.

In certain embodiments, the antibodies or antibody fragments are human or chimeric antibodies that immunospecifically bind to human PAI-1 and inhibit PAI-1 activity, such as binding of human PAI-1 to tPA, but do not inhibit the binding of human PAI-1 to vitronectin. The foregoing description of the VH and VL domains is intended to refer to all possible combinations.

In a specific embodiment, anti-PAI-1 antibody comprise:

(a) a VH CDR1 having the amino acid sequence of SEQ ID NO: 187;
(b) a VH CDR2 having the amino acid sequence of SEQ ID NO: 161;
(c) a VH CDR3 having the amino acid sequence of SEQ ID NO: 171;
(d) a VL CDR1 having the amino acid sequence of SEQ ID NO: 175;
(e) a VL CDR2 having the amino acid sequence of SEQ ID NO: 184; and
(f) a VL CDR3 having the amino acid sequence of SEQ ID NO: 185.

In a further embodiment, antibodies of the disclosure that immunospecifically binds human PAI-1 and comprise:

(a) a VH CDR1 having an amino acid sequence identical to or comprising 1, 2, or 3 amino acid residue substitutions relative to SEQ ID NO: 187;
(b) a VH CDR2 having an amino acid sequence identical to or comprising 1, 2, or 3 amino acid residue substitutions relative to SEQ ID NO: 161;
(c) a VH CDR3 having an amino acid sequence identical to or comprising 1, 2, or 3 amino acid residue substitutions relative to SEQ ID NO: 171;
(d) a VL CDR1 having an amino acid sequence identical to or comprising 1, 2, or 3 amino acid residue substitutions relative to SEQ ID NO: 175;
(e) a VL CDR2 having an amino acid sequence identical to or comprising 1, 2, or 3 amino acid residue substitutions relative to SEQ ID NO: 184; and
(f) a VL CDR3 having an amino acid sequence identical to or comprising 1, 2, or 3 amino acid residue substitutions relative to SEQ ID NO: 185, and has least two or more (at least 3, 4, 5, 6, 7, 8, 9, 10, 11, 12, etc.) of the Desired Characteristics.

In certain embodiments, anti-PAI-1 antibodies comprise:

(a) VH CDR1 having an amino acid sequence identical to or comprising 1, 2, or 3 amino acid residue substitutions relative to SEQ ID NO: 187,
(b) a VH CDR2 having an amino acid sequence identical to or comprising 1, 2, or 3 amino acid residue substitutions relative to SEQ ID NO: 161 and
(c) a VH CDR3 having an amino acid sequence identical to or comprising 1, 2, or 3 amino acid residue substitutions relative to SEQ ID NO: 171.

In another embodiment, anti-PAI-1 antibodies comprise:

(a) a VL CDR1 having an amino acid sequence identical to or comprising 1, 2, or 3 amino acid residue substitutions relative to SEQ ID NO: 176,
(b) a VL CDR2 having an amino acid sequence identical to or comprising 1, 2, or 3 amino acid residue substitutions relative to SEQ ID NO: 184 and
(c) a VL CDR3 having an amino acid sequence identical to or comprising 1, 2, or 3 amino acid residue substitutions relative to SEQ ID NO: 185.

The disclosure contemplates antibodies and antibody fragments having any combination of the foregoing VH and VL CDRs. For example, antibodies comprising:

(a) a VH CDR1 having a sequence identical to SEQ ID NO: 187,
(b) a VH CDR2 having an amino acid sequence comprising one amino acid substitution relative to SEQ ID NO: 161,
(c) a VH CDR3 having an amino acid sequence comprising one amino acid substitution relative to SEQ ID NO: 171.

By way of further example, antibodies comprising:

(a) a VL CDR1 having an amino acid sequence comprising one amino acid substitution relative to SEQ ID NO: 176,
(b) a VL CDR2 having an amino acid sequence identical to SEQ ID NO: 184, and
(c) a VL CDR3 having an amino acid sequence comprising three substitutions relative to SEQ ID NO: 185.

All other combinations are similarly contemplated. In certain embodiments, the antibodies or antibody fragments are human or chimeric antibodies that immunospecifically bind to human PAI-1 and inhibit PAI-1 activity, such as binding of human PAI-1 to tPA, but do not inhibit the binding of human PAI-1 to vitronectin. The foregoing description of the VH and VL domains is intended to refer to all possible combinations.

In certain embodiments, anti-PAI-1 antibodies comprise:

(a) a VH CDR1 having an amino acid sequence identical to or comprising 1, 2, or 3 amino acid residue substitutions relative to SEQ ID NO: 187,
(b) a VH CDR2 having an amino acid sequence identical to or comprising 1, 2, or 3 amino acid residue substitutions relative to SEQ ID NO: 161,
(c) a VH CDR3 having an amino acid sequence identical to or comprising 1, 2, or 3 amino acid residue substitutions relative to SEQ ID NO: 171,
(d) a VL CDR1 having an amino acid sequence identical to or comprising 1, 2, or 3 amino acid residue substitutions relative to SEQ ID NO: 176,
(e) a VL CDR2 having an amino acid sequence identical to or comprising 1, 2, or 3 amino acid residue substitutions relative to SEQ ID NO: 184 and
(f) a VL CDR3 having an amino acid sequence identical to or comprising 1, 2, or 3 amino acid residue substitutions relative to SEQ ID NO: 185.

The disclosure contemplates antibodies and antibody fragments having any combination of the foregoing VH and VL CDRs. For example, antibodies comprising:

(a) a VH CDR1 having a sequence identical to SEQ ID NO: 187,
(b) a VH CDR2 having an amino acid sequence comprising one amino acid substitution relative to SEQ ID NO: 161,
(c) a VH CDR3 having an amino acid sequence comprising one amino acid substitution relative to SEQ ID NO: 171.

By way of further example, antibodies comprising:

(a) a VL CDR1 having an amino acid sequence comprising one substitution relative to SEQ ID NO: 176,
(c) a VL CDR2 having an amino acid sequence identical to SEQ ID NO: 184, and a VL CDR3 having an amino acid sequence comprising three substitutions relative to SEQ ID NO: 185. All other combinations are similarly contemplated.

In certain embodiments, the antibodies or antibody fragments are human or chimeric antibodies that immunospecifically bind to human PAI-1 and inhibit PAI-1 activity, such as binding of human PAI-1 to tPA, but do not inhibit the binding of human PAI-1 to vitronectin. The foregoing description of the VH and VL domains is intended to refer to all possible combinations.

In a specific embodiment, anti-PAI-1 antibody comprise:

(a) a VH CDR1 having the amino acid sequence of SEQ ID NO: 187;
(b) a VH CDR2 having the amino acid sequence of SEQ ID NO: 161;
(c) a VH CDR3 having the amino acid sequence of SEQ ID NO: 171;
(d) a VL CDR1 having the amino acid sequence of SEQ ID NO: 176;
(e) a VL CDR2 having the amino acid sequence of SEQ ID NO: 184; and
(f) a VL CDR3 having the amino acid sequence of SEQ ID NO: 185.

In a further embodiment, antibodies of the disclosure that immunospecifically binds human PAI-1 and comprise:

(a) a VH CDR1 having an amino acid sequence identical to or comprising 1, 2, or 3 amino acid residue substitutions relative to SEQ ID NO: 187;
(b) a VH CDR2 having an amino acid sequence identical to or comprising 1, 2, or 3 amino acid residue substitutions relative to SEQ ID NO: 161;
(c) a VH CDR3 having an amino acid sequence identical to or comprising 1, 2, or 3 amino acid residue substitutions relative to SEQ ID NO: 171;
(d) a VL CDR1 having an amino acid sequence identical to or comprising 1, 2, or 3 amino acid residue substitutions relative to SEQ ID NO: 176;
(e) a VL CDR2 having an amino acid sequence identical to or comprising 1, 2, or 3 amino acid residue substitutions relative to SEQ ID NO: 184; and
(f) a VL CDR3 having an amino acid sequence identical to or comprising 1, 2, or 3 amino acid residue substitutions relative to SEQ ID NO: 185, and has least two or more (at least 3, 4, 5, 6, 7, 8, 9, 10, 11, 12, etc.) of the Desired Characteristics.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3, 4) of the following characteristics:

inhibits binding of human PAI-1 to uPA and tPA;

preferentially binds to active human PAI-1 over latent human PAI-1;

can reduce the level of VCAM-1 in dermal tissues;

can reduce the level of TNF-alpha in dermal tissues;

can stimulate plasmin-mediated activation of MMP-1.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3) of the following characteristics:

affinity (K_{D}) between 5 pm to 200 pM for active human PAI-1, as assessed by surface plasmon resonance;

immunospecifically binds to a human PAI-1:vitronectin complex, but does not disrupt the binding of human PAI-1 to vitronectin;

specifically binds to the reactive center loop of PAI-1 that interacts with tPA and uPA.

In certain embodiments, the antibody or antibody fragment has an affinity (K_{D}) of between about 5 pM and 125 pM for active human PAI-1, as assessed by surface plasmon resonance. In certain embodiments, the antibody or antibody fragment has an affinity (K_{D}) of less than or equal to 100 pM for active human PAI-1, as assessed by surface plasmon resonance. In certain embodiments, the antibody or antibody fragment has an affinity (K_{D}) of less than or equal to 50 pM for active human PAI-1, as assessed by surface plasmon resonance.

In certain embodiments, including embodiments of any of the foregoing, antibodies of the disclosure comprise

a VH CDR1 having the amino acid sequence XYAIS, wherein X is a neutral amino acid residue;

a VH CDR2 having the amino acid sequence GIIPX¹FX²TANYX³QKX⁴X⁵X⁶, wherein
X1 is a neutral amino acid residue;
X2 is a neutral or acidic amino acid residue;
X3 is a neutral amino acid residue;
X⁴ is a neutral amino residue;
X⁵ is a neutral or basic amino acid residue;
X⁶ is a neutral amino acid residue;

a VH CDR3 having the amino acid sequence EX¹RQWLEGX²FDY, wherein
X¹ is a basic amino acid residue;
X² is a basic amino acid residue;

a VL CDR1 having the amino acid sequence X¹ASEX²IYX³X⁴LA, wherein
X¹ is a basic or neutral amino acid residue;
X² is a neutral amino acid residue;
X³ is a basic amino acid residue;
X⁴ is an acidic or neutral amino acid residue;

a VL CDR2 having the amino acid sequence X¹AX²SLAS, wherein
X¹ is a basic amino acid residue;
X² is a neutral amino acid residue; and

a VL CDR3 having the amino acid sequence QQYSXYPLT, wherein X is an acidic or neutral amino acid residue.

In certain embodiments, including embodiments of any of the foregoing, antibodies of the disclosure comprise

a VH CDR1 having the amino acid sequence XYAIS, wherein X is an amino acid chosen from Ser, Thr, Gly, Ala, Val, Leu, and Be;

a VH CDR2 having the amino acid sequence GIIPX¹FX²TANYX³QKX⁴X⁵X⁶, wherein
X1 is an amino acid chosen from Ile, Ser, Thr, Gly, Ala, Val, and Leu;
X2 is an amino acid chosen from Asp, Asn, Glu, Gln, Gly, Ala, Val, Leu, Ile, Ser, Thr, and Pro;
X3 is an amino acid chosen from Ala, Ser, Thr, Gly, Ala, Val, Leu, and Ile;
X⁴ is an amino acid chosen from Phe, Leu, Ile, Ala, Ser, Thr, Gly, Ala, and Val;
X⁵ is an amino acid chosen from Gln, Asn, Arg, Lys, and His;
X⁶ is an amino acid chosen from Gly, Ser, Thr, Ala, Val, Leu, and Ile;

a VH CDR3 having the amino acid sequence EX¹RQWLEGX²FDY, wherein
X¹ is an amino acid chosen from Lys, Arg, and His;
X² is an amino acid chosen from Lys, Arg, and His;

a VL CDR1 having the amino acid sequence X¹ASEX²IYX³X⁴LA, wherein
X¹ is an amino acid chosen from Arg, Lys, His, Gln, and Asn;
X² is an amino acid chosen from Gly, Ser, Thr, Ala, Val, Leu, and Ile;
X³ is an amino acid chosen from Lys, Arg, and His;
X⁴ is an amino acid chosen from Asp, Glu, Asn, Gln, Ser, Thr, Tyr, Trp, Phe, Ala, and Gly;

a VL CDR2 having the amino acid sequence X¹AX²SLAS, wherein
X¹ is an amino acid chosen from Lys, Arg, and His;
X² is an amino acid chosen from Gly, Ser, Thr, Ala, Val, Leu, and Ile; and

a VL CDR3 having the amino acid sequence QQYSXYPLT, wherein X is an amino acid chosen from Asn, Asp, Glu, and Gln.

In certain embodiments, including embodiments of any of the foregoing, antibodies of the disclosure comprise

a VH CDR1 having the amino acid sequence XYAIS, wherein X is an amino acid chosen from Ser and Gly;

a VH CDR2 having the amino acid sequence GIIPX¹FX²TANYX³QKX⁴X⁵X⁶, wherein
X1 is an amino acid chosen from Ile, Thr, and Ala;
X2 is an amino acid chosen from Asp, Gly, Val, Ser, and Pro;
X3 is an amino acid chosen from Ala and Ser;
X⁴ is an amino acid chosen from Phe and Leu;
X⁵ is an amino acid chosen from Gln and Arg;
X⁶ is an amino acid chosen from Gly and Ser;

a VH CDR3 having the amino acid sequence EX¹RQWLEGX²FDY, wherein
X¹ is an amino acid chosen from Lys and Arg;
X² is an amino acid chosen from Arg, and His;

a VL CDR1 having the amino acid sequence X¹ASEX²IYX³X⁴LA, wherein
X¹ is an amino acid chosen from Arg and Gln;
X² is an amino acid chosen from Gly and Ser;
X³ is an amino acid chosen from Arg and His;
X⁴ is an amino acid chosen from Glu, Asn, Gln, Ser, Thr, Trp, Phe, and Ala;

a VL CDR2 having the amino acid sequence X¹AX²SLAS, wherein
X¹ is an amino acid chosen from Lys and Arg;
X² is an amino acid chosen from Ser and Thr;

a VL CDR3 having the amino acid sequence QQYSXYPLT, wherein X is an amino acid chosen from Asn and Asp.

In certain embodiments, the human or chimeric antibody or antibody fragment for use in the claimed methods immunospecifically binds to human PAI-1, inhibits human PAI-1 activity and comprises:

a VH CDR1 having the amino acid sequence of SEQ ID NO: 187;

a VH CDR2 having the amino acid sequence of SEQ ID NO: 161;

a VH CDR3 having an amino acid sequence identical to or comprising 1 or 2 amino acid residue substitutions relative to SEQ ID NO: 171;

a VL CDR1 having the amino acid sequence of SEQ ID NO: 176;

a VL CDR2 having an amino acid sequence identical to or comprising 1 or 2 amino acid residue substitutions relative of SEQ ID NO: 117; and

a VL CDR3 having an amino acid sequence identical to or comprising 1 or 2 amino acid residue substitutions relative to SEQ ID NO: 186.

For example, in certain embodiments, the antibody or antibody fragment comprises:

a VH CDR3 having the amino acid sequence EX¹RQWLEGX²FDY, wherein
X¹ is a basic amino acid residue;
X² is a basic amino acid residue.

By way of further example, in certain embodiments, the antibody or antibody fragment comprises:

a VL CDR2 having the amino acid sequence XASSLAS, wherein X is a basic amino acid residue; and

a VL CDR3 having the amino acid sequence QQYSXYPLT, wherein X is an acidic or neutral amino acid residue.

In certain embodiments, the human or chimeric antibody or antibody fragment for use in the claimed methods immunospecifically binds to human PAI-1, inhibits human PAI-1 activity and comprises:

a VH CDR1 having the amino acid sequence of SEQ ID NO: 187;

a VH CDR2 having an amino acid sequence identical to or comprising 1, 2 or 3 amino acid residue substitutions relative to SEQ ID NO: 161;

a VH CDR3 having the amino acid sequence of SEQ ID NO: 171;

a VL CDR1 having the amino acid sequence of SEQ ID NO: 176;

a VL CDR2 having an amino acid sequence identical to or comprising 1 or 2 amino acid residue substitutions relative to SEQ ID NO: 117; and

a VL CDR3 having the amino acid sequence of SEQ ID NO: 186.

For example, in certain embodiments, the antibody or antibody fragment comprises:

a VH CDR2 having the amino acid sequence GIIPX¹FX²TANYAQKFX³G, wherein
X¹ is a neutral amino acid residue;
X² is an acidic or neutral amino acid residue;
X³ is a basic or neutral amino acid residue.

By way of further example, in certain embodiments, the antibody or antibody fragment comprises:

a VL CDR2 having the amino acid sequence XASSLAS, wherein X is a basic amino acid residue.

In certain other embodiments, the human or chimeric antibody or antibody fragment for use in the claimed methods immunospecifically binds to human PAI-1, inhibits human PAI-1 activity and comprises:

a VH CDR1 having the amino acid sequence of SEQ ID NO: 187;

a VH CDR2 having the amino acid sequence of SEQ ID NO: 161;

a VH CDR3 having an amino acid sequence identical to or comprising 1 or 2 amino acid residue substitutions relative to SEQ ID NO: 171;

a VL CDR1 having an amino acid sequence identical to or comprising 1 or 2 amino acid residue substitutions relative SEQ ID NO: 176;

a VL CDR2 having the amino acid sequence of SEQ ID NO: 117; and

a VL CDR3 having the amino acid sequence of SEQ ID NO: 186.

In certain other embodiments, the human or chimeric antibody or antibody fragment for use in the claimed methods immunospecifically binds to human PAI-1, inhibits human PAI-1 activity and comprises:

a VH CDR1 having the amino acid sequence of SEQ ID NO: 187;

a VH CDR2 having the amino acid sequence of SEQ ID NO: 161;

a VH CDR3 having the amino acid sequence of SEQ ID NO: 171;

a VL CDR1 having the amino acid sequence of SEQ ID NO: 176;

a VL CDR2 having the amino acid sequence of SEQ ID NO: 117; and

a VL CDR3 having the amino acid sequence of SEQ ID NO: 186.

See, e.g., Table 2 for a representation of VH CDR1, VH CDR2, VH CDR3, VL CDR1, VL CDR2 and VL CDR3 sequences encompassed by the present disclosure which can be present in any combination to form an anti-PAI-1 antibody. Exemplary antibodies of the disclosure have such sequence (structure), bind specifically to human PAI-1 and have at least two or more (at least 3, 4, 5, 6, 7, 8, 9, 10, 11, 12, etc.) of the Desired Characteristics.

In certain embodiments, the antibody or antibody fragment has at least any number of these characteristics, such as at least 3, 4, 5, 6, 7, 8, 9, 10, 11, 12, 13, 14, 15, 16. Of course, such antibodies may optionally have additional characteristics. It should be understood that antibodies defined based on possessing any one or more of the foregoing properties possesses, at least, such one or more properties but may also possess other functional or structural characteristics. Further, it should be understood that these features may also be used to described antibodies whose CDRs are defined based on the foregoing consensus sequences. Any such antibodies may be used in the methods of the present disclosure.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3, 4) of the following characteristics:

inhibits binding of human PAI-1 to uPA and tPA;

preferentially binds to active human PAI-1 over latent human PAI-1;

can reduce the level of VCAM-1 in dermal tissues;

can reduce the level of TNF-alpha in dermal tissues;

can stimulate plasmin-mediated activation of MMP-1.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3) of the following characteristics:

affinity (K_{D}) between 5 pm to 200 pM for active human PAI-1, as assessed by surface plasmon resonance;

immunospecifically binds to a human PAI-1:vitronectin complex, but does not disrupt the binding of human PAI-1 to vitronectin;

specifically binds to the reactive center loop of PAI-1 that interacts with tPA and uPA.

In certain embodiments, the antibody or antibody fragment has an affinity (K_{D}) of between about 5 pM and 125 pM for active human PAI-1, as assessed by surface plasmon resonance. In certain embodiments, the antibody or antibody fragment has an affinity (K_{D}) of less than or equal to 100 pM for active human PAI-1, as assessed by surface plasmon resonance. In certain embodiments, the antibody or antibody fragment has an affinity (K_{D}) of less than or equal to 50 pM for active human PAI-1, as assessed by surface plasmon resonance.

The present disclosure encompasses anti-PAI-1 antibodies comprising amino acids in a sequence that is substantially the same as an amino acid sequence described herein. Amino acid sequences that are substantially the same as the sequences described herein include sequences comprising conservative amino acid substitutions, as well as amino acid deletions and/or insertions. A conservative amino acid substitution refers to the replacement of a first amino acid by a second amino acid that has chemical and/or physical properties (e.g, charge, structure, polarity, hydrophobicity/hydrophilicity) that are similar to those of the first amino acid. Conservative substitutions include replacement of one amino acid by another within the following groups: lysine (K), arginine (R) and histidine (H); aspartate (D) and glutamate (E); asparagine (N), glutamine (Q), serine (S), threonine (T), tyrosine (Y), K, R, H, D and E; alanine (A), valine (V), leucine (L), isoleucine (I), proline (P), phenylalanine (F), tryptophan (W), methionine (M), cysteine (C) and glycine (G); F, W and Y; C, S and T. Similarly contemplated is replacing a basic amino acid with another basic amino acid (e.g., replacement among Lys, Arg, His), replacing an acidic amino acid with another acidic amino acid (e.g., replacement among Asp and Glu), replacing a neutral amino acid with another neutral amino acid (e.g., replacement among Ala, Gly, Ser, Met, Thr, Leu, Ile, Asn, Gln, Phe, Cys, Pro, Trp, Tyr, Val).

The foregoing applies equally to anti-PAI antibodies and antibody fragments of the disclosure. Antibodies and antibody fragments having any one or more of the foregoing functional and structural characteristics are contemplated.

3. Framework Regions

The variable domains of the heavy and light chains each comprise four framework regions (FR1, FR2, FR3, FR4), which are the more highly conserved portions of the variable domains. The four FRs of the heavy chain are designated FR-H1, FR-H2, FR-H3 and FR-H4, and the four FRs of the light chain are designated FR-L1, FR-L2, FR-L3 and FR-L4. The Kabat numbering system is used herein, See Table 1, Kabat et al, Supra. As such, FR-H1 begins at position 1 and ends at approximately amino acid 30, FR-H2 is approximately from amino acid 36 to 49, FR-H3 is approximately from amino acid 66 to 94 and FR-H4 is approximately amino acid 103 to 113. FR-L1 begins at amino acid 1 and ends at approximately amino acid 23, FR-L2 is approximately from amino acid 35 to 49, FR-L3 is approximately from amino acid 57 to 88 and FR-L4 is approximately from amino acid 98 to 107. In certain embodiments the framework regions may contain substitutions according to the Kabat numbering system, e.g., insertion at 106A in FR-L1. In addition to naturally occurring substitutions, one or more alterations (e.g., substitutions) of FR residues may also be introduced in an anti-PAI-1 antibody. In certain embodiements, these result in an improvement or optimization in the binding affinity of the antibody for PAI-1, for example one or more of huma, mouse, or cynomolgous PAI-1. Examples of framework region residues to modify include those which non-covalently bind antigen directly (Amit et al., Science, 233:747-753 (1986)); interact with/effect the conformation of a CDR (Chothia et al., J. Mol. Biol., 196:901-917 (1987)); and/or participate in the VL-VH interface (U.S. Pat. No. 5,225,539).

In another embodiment the FR may comprise one or more amino acid changes for the purposes of “germlining”. For example, the amino acid sequences of selected antibody heavy and light chains are compared to germline heavy and light chain amino acid sequences and where certain framework residues of the selected VL and/or VH chains differ from the germline configuration (e.g., as a result of somatic mutation of the immunoglobulin genes used to prepare the phage library), it may be desirable to “backmutate” the altered framework residues of the selected antibodies to the germline configuration (i.e., change the framework amino acid sequences of the selected antibodies so that they are the same as the germline framework amino acid sequences). Such “backmutation” (or “germlining”) of framework residues can be accomplished by standard molecular biology methods for introducing specific mutations (e.g., site-directed mutagenesis; PCR-mediated mutagenesis, and the like). In one embodiment, the variable light and/or heavy chain framework residues are backmutated. In another embodiment, the variable heavy chain of an antibody of the disclosure is backmutated. In another embodiment, the variable heavy chain of an antibody of the disclosure comprises at least one, at least two, at least three, at least four or more backmutations.

In certain embodiments, the VH of an anti-PAI-1 antibody of the disclosure may comprise FR1, FR2, FR3 and/or FR4 that has an amino acid sequence identity with the corresponding framework regions (i.e., FR1 of antibody X as compared to FR1 of antibody Y) of any one or more of the VH chains of the anti-PAI antibodies described herein and set forth in the Sequence Listing, that is from about 65% to about 100%. In one embodiment, the anti-PAI-1 antibodies comprise a VH FR amino acid sequence (FR1, FR2, FR3 and/or FR4) having at least 65%, 70%, 75%, 80%, 85%, 90%, 95% or at least 100% sequence identity with the corresponding FR of the VH set forth in any one or more of SEQ ID NOs: 2, 6, 10, 34, 66, 82, or 110. In a further embodiment the anti-PAI-1 antibodies comprise a VH FR amino acid sequence (FR1, FR2, FR3 and/or FR4) having at least 90%, 91%, 92%, 93%, 94%, 95%, 96%, 97%, 98%, 99% or having at least 100% sequence identity with the corresponding FR of the VH set forth in any one or more of SEQ ID NOs: 2, 6, 10, 34, 66, 82, or 110.

In certain embodiments, the anti-PAI-1 antibodies may comprise a VH FR (FR1, FR2, FR3 and/or FR4) having an amino acid sequence identical to or comprising 1, 2 or 3 amino acid substitutions relative to the corresponding FR of the VH set forth in any one or more of SEQ ID NOs: 2, 6, 10, 34, 66, 82, or 110. In particular FR1, FR2, FR3 or FR4 of the VH may each have an amino acid sequence identical to or comprising 1, 2 or 3 amino acid substitutions relative to the corresponding FR1, FR2, FR3 or FR4 of the VH set forth in any one or more of SEQ ID NOs: 2, 6, 10, 34, 66, 82, or 110.

In certain embodiments, the VL of an anti-PAI-1 antibody of the disclosure may comprise FR1, FR2, FR3 and/or FR4 that has an amino acid sequence identity with the corresponding framework regions (i.e., FR1 of antibody X as compared to FR1 of antibody Y) of any one or more of the VH chains of the anti-PAI antibodies described herein and set forth in the Sequence Listing, that is from about 65% to about 100%. In one embodiment, the anti-PAI-1 antibodies comprise a VL FR amino acid sequence (FR1, FR2, FR3 and/or FR4) having at least 65%, 70%, 75%, 80%, 85%, 90%, 95% or at least 100% sequence identity with the corresponding FR of the VL chains set forth in any one or more of SEQ ID NOs: 4, 8, 12, 36, 68, 84, or 112. In a further embodiment the anti-PAI-1 antibodies comprise a VL FR amino acid sequence (FR1, FR2, FR3 and/or FR4) having at least 90%, 91%, 92%, 93%, 94%, 95%, 96%, 97%, 98%, 99% or having at least 100% sequence identity with the corresponding FR of the VL chains set forth in any one or more of SEQ ID NOs: 4, 8, 12, 36, 68, 84, or 112.

In certain embodiments, the anti-PAI-1 antibodies may comprise a VL FR (FR1, FR2, FR3 and/or FR4) having an amino acid sequence identical to or comprising 1, 2 or 3 amino acid substitutions relative to the corresponding FR of the VL chains set forth in any one or more of SEQ ID NOs: 4, 8, 12, 36, 68, 84, or 112. In particular FR1, FR2, FR3 or FR4 of the VL may each have an amino acid sequence identical to or comprising 1, 2 or 3 amino acid substitutions relative to the corresponding FR of the VL chains set forth in any one or more of SEQ ID NOs: 4, 8, 12, 36, 68, 84, or 112.

In a further embodiment, of any of the foregoing, the antibodies of the disclosure immunospecifically binds human PAI-1, have frameworks as described above, and have one or more of the Desired Characteristics

In certain embodiments, the antibody or antibody fragment has at least any number of these characteristics, such as at least 3, 4, 5, 6, 7, 8, 9, 10, 11, 12, 13, 14, 15, 16. Of course, such antibodies may optionally have additional characteristics. It should be understood that antibodies defined based on possessing any one or more of the foregoing properties possesses, at least, such one or more properties but may also possess other functional or structural characteristics.

The foregoing applies equally to antibodies and antibody fragments of the present disclosure. Moreover, the disclosure contemplates antibodies and antibody fragments having any one or more of the functional and structural properties described in the applications, and further comprising any combination of VH FR1, FR2, FR3, and FR4 regions described herein. Moreover, the disclosure contemplates antibodies and antibody fragments having any one or more of the functional and structural properties described in the applications, and further comprising any combination of VL FR1, FR2, FR3, and FR4 regions described herein, as well as antibodies and antibody fragments having any combination of VH FR1, FR2, FR3, FR4 regions, and VL FR1, FR2, FR3, and FR4 regions. Moreover, the disclosure contemplates antibodies and antibody fragments comprising any combination of VL FR1, FR2, FR3, and FR4 regions described herein, as well as antibodies and antibody fragments having any combination of VH FR1, FR2, FR3, FR4 regions, and VL FR1, FR2, FR3, and FR4 regions. In certain embodiments, said antibody is a human, humanized or chimeric antibody.

The foregoing description applies equally to antibodies and antibody fragments of the disclosure. Moreover, the disclosure contemplates antibodies and antibody fragments having any combination of the structural and/or functional features described herein. Further, the various CDR and FR domains (CDRs 1-3 of the VH domain, CDRs 1-3 of the VL domain, FRs 1-4 of the VH domain, FRs 1-4 of the VL domain) described above can be defined with other features of the disclosure described herein. Any such anti-PAI-1 antibodies and antibody fragments can be used in any of the methods described herein.

Note that certain of the CDR and FR regions provided in the Sequence Listing may correspond to the same amino acid sequence (e.g., VH CDR2 from two different antibodies identified in the examples may have the same sequence). They are provided separately to illustrate the experimental results, as well as the relationships among certain antibodies identified. However, when a sequence identifier is used to describe the sequence of a particular antibody, it should be understood, unless otherwise specified, to refer to the underlying sequence itself without requiring or assuming a source of that sequence.

4. Nucleotide Sequences Encoding Anti-PAI-1 Antibodies

In addition to the amino acid sequences described above, the disclosure further provides nucleotide sequences corresponding to the amino acid sequences and encoding for the human, humanized and/or chimeric antibodies of the disclosure. In one embodiment, the disclosure provides polynucleotides comprising a nucleotide sequence encoding an anti-PAI-1 antibody described herein or fragments thereof. These include, but are not limited to, nucleotide sequences that code for the above referenced amino acid sequences. Thus, the present disclosure also provides polynucleotide sequences encoding VH and VL domain regions including CDRs and FRs of antibodies described herein as well as expression vectors for their efficient expression in cells (e.g. mammalian cells). Methods of making the anti-PAI-1 antibodies using polynucleotides are described below in more detail and are known in the art. The foregoing polynucleotides encode anti-PAI-1 antibodies having the structural and/or functional features described herein. For example, such antibodies bind immunspecifically to human PAI-1 and mouse PAI-1.

The disclosure also encompasses polynucleotides that hybridize under stringent or lower stringency hybridization conditions, e.g., as defined herein, to polynucleotides that encode an antibody of the disclosure described herein that binds to PAI-1. The term “stringency” as used herein refers to experimental conditions (e.g. temperature and salt concentration) of a hybridization experiment to denote the degree of homology between the probe and the filter bound nucleic acid; the higher the stringency, the higher percent homology between the probe and filter bound nucleic acid.

Stringent hybridization conditions include, but are not limited to, hybridization to filter-bound DNA in 6× sodium chloride/sodium citrate (SSC) at about 45° C. followed by one or more washes in 0.2×SSC/0.1% SDS at about 50-65° C., highly stringent conditions such as hybridization to filter-bound DNA in 6×SSC at about 45° C. followed by one or more washes in 0.1×SSC/0.2% SDS at about 65° C., or any other stringent hybridization conditions known to those skilled in the art (see, for example, Ausubel, F. M. et al., eds. 1989 Current Protocols in Molecular Biology, vol. 1, Green Publishing Associates, Inc. and John Wiley and Sons, Inc., NY at pages 6.3.1 to 6.3.6 and 2.10.3).

In certain embodiments, the polynucleotide sequences of the disclosure may also comprise a nucleotide sequence encoding an anti-PAI-1 antibody VH that hybridizes under stringent conditions to the nucleotide sequence of SEQ ID NO: 5 and/or any of the particular anti-PAI-1 antibody VH sequences provided in the tables and Sequence Listing. In another embodiment, the polynucleotide sequences of the disclosure may also comprise a nucleotide sequence encoding an anti-PAI-1 antibody VL that hybridizes under stringent conditions to the nucleotide sequence of SEQ ID NO: 7 and/or any of the particular anti-PAI-1 antibody VL sequences provided in the tables and Sequence Listing.

In certain embodiments, the polynucleotide sequences of the disclosure may also comprise a nucleotide sequence encoding an anti-PAI-1 antibody VH at least 65%, 70%, 75%, 80%, 85%, 90%, 95% or having about 100% identity to the nucleotide sequence of SEQ ID NO: 5 and/or any of the particular anti-PAI-1 antibody VH sequences provided in the tables and Sequence Listing. In one embodiment, the anti-PAI-1 antibodies have a VH nucleotide sequence having at least, 90%, 91%, 92%, 93%, 94%, 95%, 96%, 97%, 98%, 99% or having about 100% identity to the nucleotide sequence of SEQ ID NO: 5 and/or any of the particular anti-PAI-1 antibody VH sequences provided in the tables and Sequence Listing.

In certain embodiments, the polynucleotide sequences of the disclosure may also comprise a nucleotide sequence encoding an anti-PAI-1 antibody VL at least 65%, 70%, 75%, 80%, 85%, 90%, 95% or having about 100% identity to the nucleotide sequence of SEQ ID NO:7 and/or any of the particular anti-PAI-1 antibody VL sequences provided in the tables and Sequence Listing. In one embodiment, the anti-PAI-1 antibodies have a VL nucleotide sequence having at least, 90%, 91%, 92%, 93%, 94%, 95%, 96%, 97%, 98%, 99% or having about 100% identity to the nucleotide sequence of SEQ ID NO: 7 and/or any of the particular anti-PAI-1 antibody VL sequences provided in the tables and Sequence Listing.

Substantially identical sequences may be polymorphic sequences, i.e., alternative sequences or alleles in a population. An allelic difference may be as small as one base pair. Substantially identical sequences may also comprise mutagenized sequences, including sequences comprising silent mutations. A mutation may comprise one or more residue changes, a deletion of one or more residues, or an insertion of one or more additional residues.

The polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

A polynucleotide encoding an antibody may also be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably polyA+ RNA, isolated from, any tissue or cells expressing the antibody, such as hybridoma cells selected to express an antibody) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence and corresponding amino acid sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY), to generate antibodies having a different amino acid sequence, for example to create amino acid substitutions, deletions, and/or insertions.

5. Biological Characteristics of the Anti-PAI-1 Antibodies

An antibody having a biological characteristic of a designated antibody is one which possesses one or more of the biological characteristics of that antibody which distinguish it from other antibodies that bind to the same antigen, PAI-1. As used here, “biological characteristics” of an antibody refers to any one of biochemical, binding and functional characteristics, which are used to select antibodies for therapeutic, research, and diagnostic uses described below.

The biochemical characteristics of the antibodies of the disclosure include, but are not limited to, isoelectric point (pI) and melting temperature (Tm). The binding characteristics of the antibodies of the disclosure include, but are not limited to, binding specificity, dissociation constant (Kd), or its inverse, association constant (Ka), or its component kon or koff rates, epitope, ability to distinguish between various forms and/or preparations of PAI-1 (e.g., recombinant, native, acetylated) and ability to bind soluble and/or immobilized antigen. The functional characteristics of the antibodies of the present disclosure include, but are not limited to; inhibition of PAI-1 activity, ability to bind active PAI-1 preferentially (in some instances) to latent PAI-1, ability to bind the PAI-1:vitronectin complex, ability to inhibit binding of PAI-1 to tPA, cross-reactivity with PAI-1 from one or more non-human species, lack of cross-reactivity with PAI-2 and PAI-3, etc. Described herein are the antibodies of the disclosure and their respective characteristics along with methods for the modification and fine tuning of those characteristics. Methods for measuring the characteristics of the antibodies are well known in the art, some of which are detailed below and in the examples.

a) Biochemical Characteristics

Antibodies like all polypeptides have an Isoelectric Point (pI), which is generally defined as the pH at which a polypeptide carries no net charge. It is known in the art that protein solubility is typically lowest when the pH of the solution is equal to the isoelectric point (pI) of the protein. As used herein the pI value is defined as the pI of the predominant charge form. The pI of a protein may be determined by a variety of methods including but not limited to, isoelectric focusing and various computer algorithms (see, e.g., Bjellqvist et al., 1993, Electrophoresis 14:1023). In addition, the thermal melting temperatures (Tm) of the Fab domain of an antibody, can be a good indicator of the thermal stability of an antibody and may further provide an indication of the shelf-life. A lower Tm indicates more aggregation/less stability, whereas a higher Tm indicates less aggregation/more stability. Thus, in certain embodiments antibodies having higher Tm are preferable. Tm of a protein domain (e.g., a Fab domain) can be measured using any standard method known in the art, for example, by differential scanning calorimetry (see, e.g., Vermeer et al., 2000, Biophys. J. 78:394-404; Vermeer et al., 2000, Biophys. J. 79: 2150-2154).

Accordingly, in certain embodiments the present disclosure includes anti-PAI-1 antibodies of the disclosure that have certain preferred biochemical characteristics such as a particular isoelectric point (pI) or melting temperature (Tm).

More specifically, in one embodiment, the anti-PAI-1 antibodies of the present disclosure have a pI ranging from 5.5 to 9.5. In still another specific embodiment, the anti-PAI-1 antibodies of the present disclosure have a pI that ranges from about 5.5 to about 6.0, or about 6.0 to about 6.5, or about 6.5 to about 7.0, or about 7.0 to about 7.5, or about 7.5 to about 8.0, or about 8.0 to about 8.5, or about 8.5 to about 9.0, or about 9.0 to about 9.5. In other specific embodiments, the anti-PAI-1 antibodies of the present disclosure have a pI that ranges from 5.5-6.0, or 6.0 to 6.5, or 6.5 to 7.0, or 7.0-7.5, or 7.5-8.0, or 8.0-8.5, or 8.5-9.0, or 9.0-9.5. Even more specifically, the anti-PAI-1 antibodies of the present disclosure have a pI of at least 5.5, or at least 6.0, or at least 6.3, or at least 6.5, or at least 6.7, or at least 6.9, or at least 7.1, or at least 7.3, or at least 7.5, or at least 7.7, or at least 7.9, or at least 8.1, or at least 8.3, or at least 8.5, or at least 8.7, or at least 8.9, or at least 9.1, or at least 9.3, or at least 9.5. In other specific embodiments, the anti-PAI-1 antibodies of the present disclosure have a pI of at least about 5.5, or at least about 6.0, or at least about 6.3, or at least about 6.5, or at least about 6.7, or at least about 6.9, or at least about 7.1, or at least about 7.3, or at least about 7.5, or at least about 7.7, or at least about 7.9, or at least about 8.1, or at least about 8.3, or at least about 8.5, or at least about 8.7, or at least about 8.9, or at least about 9.1, or at least about 9.3, or at least about 9.5.

It is possible to optimize solubility by altering the number and location of ionizable residues in the antibody to adjust the pI. For example the pI of a polypeptide can be manipulated by making the appropriate amino acid substitutions (e.g., by substituting a charged amino acid such as a lysine, for an uncharged residue such as alanine). Without wishing to be bound by any particular theory, amino acid substitutions of an antibody that result in changes of the pI of said antibody may improve solubility and/or the stability of the antibody. One skilled in the art would understand which amino acid substitutions would be most appropriate for a particular antibody to achieve a desired pI. In one embodiment, a substitution is generated in an antibody of the disclosure to alter the pI. It is specifically contemplated that the substitution(s) of the Fc region that result in altered binding to FcγR (described supra) may also result in a change in the pI. In another embodiment, substitution(s) of the Fc region are specifically chosen to effect both the desired alteration in FcγR binding and any desired change in pI.

In one embodiment, the ant-PAI-1 antibodies of the present disclosure have a Tm ranging from 65° C. to 120° C. In specific embodiments, the ant-PAI-1 antibodies of the present disclosure have a Tm ranging from about 75° C. to about 120° C., or about 75° C. to about 85° C., or about 85° C. to about 95° C., or about 95° C. to about 105° C., or about 105° C. to about 115° C., or about 115° C. to about 120° C. In other specific embodiments, the ant-PAI-1 antibodies of the present disclosure have a Tm ranging from 75° C. to 120° C., or 75° C. to 85° C., or 85° C. to 95° C., or 95° C. to 105° C., or 105° C. to 115° C., or 115° C. to 120° C. In still other specific embodiments, the ant-PAI-1 antibodies of the present disclosure have a Tm of at least about 65° C., or at least about 70° C., or at least about 75° C., or at least about 80° C., or at least about 85° C., or at least about 90° C., or at least about 95° C., or at least about 100° C., or at least about 105° C., or at least about 110° C., or at least about 115° C., or at least about 120° C. In yet other specific embodiments, the ant-PAI-1 antibodies of the present disclosure have a Tm of at least 65° C., or at least 70° C., or at least 75° C., or at least 80° C., or at least 85° C., or at least 90° C., or at least 95° C., or at least 100° C., or at least 105° C., or at least 110° C., or at least 115° C., or at least 120° C.

b) Binding Characteristics

As described above, the anti-PAI-1 antibodies of the disclosure immunospecifically bind at least one specified epitope or antigenic determinants of the PAI-1 protein, peptide, subunit, fragment, portion or any combination thereof either exclusively or preferentially with respect to other polypeptides. The term “epitope” or “antigenic determinant” as used herein refers to a protein determinant capable of binding to an antibody, wherein the term “binding” herein preferably relates to a specific binding. These protein determinants or epitopes usually consist of chemically active surface groupings of molecules such as amino acids or sugar side chains and usually have a specific three dimensional structural characteristics, as well as specific charge characteristics. Conformational and non-conformational epitopes are distinguished in that the binding to the former but not the latter is lost in the presence of denaturing solvents. The term “discontinuous epitope” as used herein, refers to a conformational epitope on a protein antigen which is formed from at least two separate regions in the primary sequence of the protein.

In certain embodiments, the anti-PAI-1 antibodies immunospecifically bind to human PAI-1 and antigenic fragments thereof. In one embodiment, the anti-PAI antibodies specifically bind to reactive center loop of PAI-1. In certain embodiments, the anti-PAI-1 antibodies bind the same epitope as Antibody 8. In certain embodiments, the anti-PAI-1 antibodies bind the same epitope as an antibody comprising the 6 CDRs of Antibody 8. In certain embodiments, the anti-PAI-1 antibodies bind the same epitope as an antibody comprising the three CDRs of Antibody 8 variable heavy chain.

In certain embodiments, the antibodies of the disclosure may bind epitopes conserved across species. In one embodiment, antibodies of the disclosure bind murine, non-human primate, rat, bovine, pig or other mammalian PAI-1 and antigenic fragments thereof. In one embodiment the antibodies of the disclosure may bind to one or more PAI-1 orthologs and or isoforms. In a specific embodiment, antibodies of the disclosure bind to PAI-1 and antigenic fragments thereof from one or more species, including, but not limited to, mouse, rat, monkey, primate, and human. In certain embodiments, the antibodies of the disclosure may bind an eptiope within humans across PAI-1 homologs and/or isoforms and/or conformational variants and/or subtypes.

The interactions between antigens and antibodies are the same as for other non-covalent protein-protein interactions. In general, four types of binding interactions exist between antigens and antibodies: (i) hydrogen bonds, (ii) dispersion forces, (iii) electrostatic forces between Lewis acids and Lewis bases, and (iv) hydrophobic interactions. Hydrophobic interactions are a major driving force for the antibody-antigen interaction, and are based on repulsion of water by non-polar groups rather than attraction of molecules (Tanford, 1978). However, certain physical forces also contribute to antigen-antibody binding, for example, the fit or complimentary of epitope shapes with different antibody binding sites. Moreover, other materials and antigens may cross-react with an antibody, thereby competing for available free antibody.

Measurement of the affinity constant and specificity of binding between antigen and antibody is a pivotal element in determining the efficacy of therapeutic, diagnostic and research methods using the anti-PAI-1 antibodies. “Binding affinity” generally refers to the strength of the sum total of the noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the equilibrium dissociation constant (Kd), which is calculated as the ratio k_{off}/kₒₙ. See, e.g., Chen, Y., et al., (1999) J. Mol. Biol 293:865-881. Affinity can be measured by common methods known in the art, including those described and exemplified herein, such as BIACORE™. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure.

The present anti-PAI-1 antibodies have binding affinities for a PAI-1 epitope that include a dissociation constant (K_{d}) of less than 1×10⁻²M, 1×10⁻³M, 1×10⁻⁴M, 1×10⁻⁵M, 1×10⁻⁶M, 1×10⁻⁷M, 1×10⁻⁸M, 1×10⁻⁹M, 1×10⁻¹⁰M, 1×10⁻¹¹M, 1×10⁻¹²M, 1×10⁻¹³M, 1×10⁻¹⁴M or less than 1×10⁻¹⁵M. In one embodiment, the anti-PAI-1 antibodies have a K_{d} of less than 10⁻⁷M, less than 5×10⁻⁸M, less than 10⁻⁸M, less than 5×10⁻⁹M, less than 10⁻⁹M, less than 5×10⁻¹⁰M, less than 10⁻¹⁰M, less than 5×10⁻¹¹M, less than 10⁻¹¹M, less than 5×10⁻¹²M, less than 10⁻¹²M, less than 5×10⁻¹³M, less than 10⁻¹³M, less than 5×10⁻¹⁴M, less than 10⁻¹⁴M, less than 5×10⁻¹⁵M, or less than 10⁻¹⁵ M. In certain embodiments, anti-PAI-1 antibodies have binding affinities for a PAI-1 epitope that include a dissociation constant (K_{d}) of between 1×10⁻⁶M and 1×10⁻¹⁰M, 1×10⁻⁶M and 1×10⁻¹¹M, 1×10⁻⁶M and 1×10⁻¹²M, 1×10⁻⁶M and 1×10⁻¹³M, 1×10⁻⁶M and 1×10⁻¹⁵M, 1×10⁻⁶M and 1×10⁻¹⁵M, 1×10⁻⁷M and 1×10⁻¹⁰M, 1×10⁻⁷M and 1×10⁻¹¹M, 1×10⁻⁷M and 1×10⁻¹²M, 1×10⁻⁷M and 1×10⁻¹³M, 1×10⁻⁷M and 1×10⁻¹⁴M, 1×10⁻⁷M and 1×10⁻¹⁵M, 1×10⁻⁸M and 1×10⁻¹⁰M, 1×10⁻⁸M and 1×10⁻¹¹M, 1×10⁻⁸M and 1×10⁻¹²M, 1×10⁻⁸M and 1×10⁻¹³M, 1×10⁻⁸M and 1×10⁻¹⁴M, 1×10⁻⁸M and 1×10⁻¹⁵M, 1×10⁻⁹M and 1×10⁻¹⁰M, 1×10⁻⁹M and 1×10⁻¹¹M, 1×10⁻⁹M and 1×10⁻¹²M, 1×10⁻⁹M and 1×10⁻¹³M, 1×10⁻⁹M and 1×10⁻¹⁴M and 1×10⁻⁹M and 1×10⁻¹⁵M. In certain embodiments, K_{d} is measured by BIACORE™ affinity data. In certain embodiments, K_{d} is measured by cell binding.

In certain embodiments, the anti-PAI-1 antibodies are high-affinity antibodies. By “high-affinity antibody” is meant an antibody which binds to a PAI-1 epitope with an affinity less than 10⁻⁸M (e.g., 10⁻⁹M, 10⁻¹⁰M, etc.).

In certain embodiments, the anti-PAI-1 antibodies have an affinity between 5 pM and 200 pM for active human PAI-1, as assessed by plasmon resonance. In certain embodiments, the affinity is approximately 5, 10, 15, 20, 25, 50, 60, 70, 75, 80, 90, 100, etc. pM. In certain embodiments, the affinity is between about 5 pM and 50 pM. In certain embodiments, the affinity is between about 5 pM and 100 pM.

In certain embodiments, the anti-PAI-1 antibodies are described as having a binding affinity of a specific molarity or better. “Or better” when used herein refers to a stronger binding, represented by a smaller numerical Kd value. For example, an antibody which has an affinity for an antigen of “0.6 nM or better”, the antibody's affinity for the antigen is <0.6 nM, i.e. 0.59 nM, 0.58 nM, 0.57 nM etc. or any value less than 0.6 nM.

In an alternative embodiment, the affinity of the anti-PAI-1 antibodies is described in terms of the association constant (Kₐ), which is calculated as the ratio kₒₙ/k_{off}. In this instance the present anti-PAI-1 antibodies have binding affinities for a PAI-1 epitope that include an association constant (Kₐ) of at least 1×10²M⁻¹, 1×10³M⁻¹, 1×10⁴M⁻¹, 1×10⁵M⁻¹, 1×10⁶M⁻¹, 1×10⁷M⁻¹, 1×10⁸M⁻¹, 1×10⁹M⁻¹, 1×10¹⁰M⁻¹ 1×10¹¹M⁻¹ 1×10¹²M⁻¹, 1×10¹³M⁻¹, 1×10¹⁴M⁻¹ or at least 1×10¹⁵M⁻¹. In one embodiment, the anti-PAI-1 antibodies have a Kₐ of at least 10⁷ M⁻¹, at least 5×10⁷M⁻¹, at least 10⁸M⁻¹, at least 5×10⁸M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹²M⁻¹, at least 5×10¹² M⁻¹, at least 10¹³M⁻¹, at least 5×10¹¹ M⁻¹, at least 10¹⁴ M⁻¹, at least 5×10¹⁴ M⁻¹, at least 10¹⁵ M⁻¹, or at least 5×10¹⁵ M. In certain embodiments, anti-PAI-1 antibodies have binding affinities for a PAI-1 epitope that include an association constant (Kₐ) of between 1×10²M⁻¹ and 1×10³M⁻¹, 1×10²M⁻¹ and 1×10⁴M⁻¹, 1×10²M⁻¹ and 1×10⁵M⁻¹, 1×10²M⁻¹ and 1×10⁶M⁻¹, 1×10³M⁻¹ and 1×10⁴M⁻¹, 1×10³M⁻¹ and 1×10⁵M⁻¹, 1×10³M⁻¹ and 1×10⁶M⁻¹, 1×10⁴M⁻¹ and 1×10⁵M⁻¹, 1×10⁴M⁻¹ and 1×10⁶M⁻¹ and 1×10⁵M⁻¹ and 1×10⁶M⁻¹.

In certain embodiments the rate at which the anti-PAI-1 antibodies dissociates from a PAI-1 epitope may be more relevant than the value of the K_{d} or the Kₐ. In this instance the present anti-PAI-1 antibodies bind to a PAI-1 with a k_{off} of less than 10⁻² s⁻¹, less than 10⁻³ s⁻¹, less than 5×10⁻³ s⁻¹, less than 10⁻⁴ s⁻¹, less than 5×10⁻⁴ s⁻¹, less than 10⁻⁵ s⁻¹, less than 5×10⁻⁵ s⁻¹, less than 10⁻⁶ s⁻¹, less than 5×10⁻⁶ s⁻¹, less than 10⁻⁷ s⁻¹, less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹, or less than 10⁻¹⁰ s⁻¹. In certain other embodiments the rate at which the anti-PAI-1 antibodies associate with a PAI-1 epitope may be more relevant than the value of the K_{d} or the Kₐ. In this instance the present anti-PAI-1 antibodies bind to a PAI-1 with a kₒₙ rate of at least 10⁵ M⁻¹s⁻¹, at least 5×10⁵M⁻¹s⁻¹, at least 10⁶ M⁻¹s⁻¹, at least 5×10⁶M⁻¹s⁻¹, at least 10⁷M⁻¹s⁻¹, at least 5×10⁷M⁻¹s⁻¹, or at least 10⁸M⁻¹s⁻¹, or at least 10⁹M⁻¹s⁻¹.

Determination of binding affinity can be measured using the specific techniques described further in the Example section, and methods well known in the art. One such method includes measuring the disassociation constant “Kd” by a radiolabeled antigen binding assay (RIA) performed with the Fab version of an antibody of interest and its antigen as described by the following assay that measures solution binding affinity of Fabs for antigen by equilibrating Fab with a minimal concentration of (¹²⁵I)-labeled antigen in the presence of a titration series of unlabeled antigen, then capturing bound antigen with an anti-Fab antibody-coated plate (Chen, et al., (1999) J. Mol. Biol 293:865-881). To establish conditions for the assay, microtiter plates (Dynex) are coated overnight with 5 μg/ml of a capturing anti-Fab antibody (Cappel Labs) in 50 mM sodium carbonate (H 9.6), and subsequently blocked with 2% (w/v) bovine serum albumin in PBS for two to five hours at room temperature (approximately 23° C.). In a non-adsorbant plate (Nunc #269620), 100 pM or 26 pM [¹²⁵I]-antigen are mixed with serial dilutions of a Fab of interest (e.g., consistent with assessment of an anti-VEGF antibody, Fab-12, in Presta et al., (1997) Cancer Res. 57:4593-4599). The Fab of interest is then incubated overnight; however, the incubation may continue for a longer period (e.g., 65 hours) to insure that equilibrium is reached. Thereafter, the mixtures are transferred to the capture plate for incubation at room temperature (e.g., for one hour). The solution is then removed and the plate washed eight times with 0.1% Tween-20 in PBS. When the plates have dried, 150 μl/well of scintillant (MicroScint-20; Packard) is added, and the plates are counted on a Topcount gamma counter (Packard) for ten minutes. Concentrations of each Fab that give less than or equal to 20% of maximal binding are chosen for use in competitive binding assays.

In another instance the Kd value may be measured by using surface plasmon resonance assays using a BIACORE™-2000 or a BIACORE™-3000 (BIAcore, Inc., Piscataway, N.J.) at 25° C. with immobilized antigen CM5 chips at ^{˜}10 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 110 mM sodium acetate, pH 4.8, into 5 ug/ml (^{˜}0.2 uM) before injection at a flow rate of 5 ul/minute to achieve approximately 10 response units (RU) of coupled protein. Following the injection of antigen, 1M ethanolamine is injected to block unreacted groups. For kinetics measurements, two-fold serial dilutions of Fab (0.78 nM to 500 nM) are injected in PBS with 0.05% Tween 20 (PBST) at 25° C. at a flow rate of approximately 25 ul/min. Association rates (kₒₙ) and dissociation rates (k_{off}) are calculated using a simple one-to-one Langmuir binding model (BIACORE™ Evaluation Software version 3.2) by simultaneously fitting the association and dissociation sensorgram.

If the on-rate exceeds 10⁶M⁻¹ S⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-Aminco spectrophotometer (ThermoSpectronic) with a stir red cuvette. An “on-rate” or “rate of association” or “association rate” or “kₒₙ” according to this disclosure can also be determined with the same surface plasmon resonance technique described above using a BIACORE™-2000 or a BIACORE™-3000 (BIAcore, Inc., Piscataway, N.J.) as described above.

Methods and reagents suitable for determination of binding characteristics of an antibody of the present disclosure, or an altered/mutant derivative thereof (discussed below), are known in the art and/or are commercially available (U.S. Pat. Nos. 6,849,425; 6,632,926; 6,294,391; 6,143,574). Moreover, equipment and software designed for such kinetic analyses are commercially available (e.g. BIACORE® A100, and BIACORE® 2000 instruments; Biacore International AB, Uppsala, Sweden).

In one embodiment, a binding assay may be performed either as direct binding assays or as competition-binding assays. Binding can be detected using standard ELISA or standard Flow Cytometry assays. In a direct binding assay, a candidate antibody is tested for binding to PAI-1 antigen. Competition-binding assay, on the other hand, assess the ability of a candidate antibody to compete with a known anti-PAI-1 antibody or other compound that binds PAI-1. In general any method that permits the binding of an antibody with a PAI-1 that can be detected is encompassed with the scope of the present disclosure for detecting and measuring the binding characteristics of the antibodies. One of skill in the art will recognize these well known methods and for this reason are not provided in detail here. These methods are also utilized to screen a panel of antibodies for those providing the desired characteristics.

In certain embodiments an antibody of the disclosure immunospecifically binds to human PAI-1 and has one or more of the characteristics set forth in the examples vis-a-vis affinity, specificity, neutralization capacity (inhibition of PAI-1 bioactivty), epitope binding, etc. In certain embodiments, an antibody of the disclosure immunospecifically binds to human PAI-1 and has one or more of the Desired Characteristics

In certain embodiments, the antibody or antibody fragment has at least any number of these characteristics, such as at least 3, 4, 5, 6, 7, 8, 9, 10, 11, 12, 13, 14, 15, 16. Of course, such antibodies may optionally have additional characteristics. It should be understood that antibodies defined based on possessing any one or more of the foregoing properties possesses, at least, such one or more properties but may also possess other functional or structural characteristics.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3, 4) of the following characteristics:

inhibits binding of human PAI-1 to uPA and tPA;

preferentially binds to active human PAI-1 over latent human PAI-1;

can reduce the level of VCAM-1 in dermal tissues;

can reduce the level of TNF-alpha in dermal tissues;

can stimulate plasmin-mediated activation of MMP-1.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3, 4) of the following characteristics:

affinity (K_{D}) between 5 pm to 200 pM for active human PAI-1, as assessed by surface plasmon resonance;

immunospecifically binds to a human PAI-1:vitronectin complex, but does not disrupt the binding of human PAI-1 to vitronectin;

specifically binds to the reactive center loop of PAI-1 that interacts with tPA and uPA.

In certain embodiments, the antibody or antibody fragment has an affinity (K_{D}) of between about 5 pM and 125 pM for active human PAI-1, as assessed by surface plasmon resonance. In certain embodiments, the antibody or antibody fragment has an affinity (K_{D}) of less than or equal to 100 pM for active human PAI-1, as assessed by surface plasmon resonance. In certain embodiments, the antibody or antibody fragment has an affinity (K_{D}) of less than or equal to 50 pM for active human PAI-1, as assessed by surface plasmon resonance. It should be understood that antibodies defined based on possessing any one or more of the foregoing properties possesses, at least, such one or more properties but may also possess other functional or structural characteristics.

c) Functional Characteristics

In certain embodiments, the anti-PAI-1 antibodies of the disclosure inhibit human PAI-1 activity, such as binding of human PAI-1 to tPA. This can be measured in cell based or cell free systems, as set forth in the example. In one embodiment, the anti-PAI-1 antibodies of the disclosure neutralize a biological activity of PAI-1, particularly human PAI-1.

Neutralization assays are performed using methods known in the art using, some of which are described in the example. The neutralization of PAI-1, such as human PAI-1, is measured with an IC50 of 1×10⁻⁶ M or less, 1×10⁻⁷ M or less, 1×10⁻⁸ M or less, 1×10⁻⁹ M or less, 1×10⁻¹⁰ M or less and 1×10⁻¹¹ M or less. In certain embodiments, the neutralization of PAI-1 is measured with an IC50 of between 1×10⁻⁶ M and 1×10⁻⁹ M, 1×10⁻⁶M and 1×10⁻¹⁰ M, 1×10⁻⁶ M and 1×10⁻¹¹ M^{,} 1×10⁻⁷ M and 1×10⁻⁹ M, 1×10⁻⁷M and 1×10⁻¹⁰ M, 1×10⁻⁷ M and 1×10⁻¹¹ M, 1×10⁻⁸ M and 1×10⁻⁹ M, 1×10⁻⁸ M and 1×10⁻¹⁰ M and 1×10⁻⁸ M and 1×10⁻¹¹ M. In a further embodiment, the anti-PAI-1 antibodies neutralize at least one of (one or more of) chimpanzee PAI-1, baboon PAI-1, marmoset PAI-1, cynomolgus PAI-1, rhesus PAI-1, rat PAI-1, mouse PAI-1, pig PAI-1 or other mammalian PAI-1. The term “inhibitory concentration 50%” (abbreviated as “IC50”) represents the concentration of an inhibitor (e.g., an anti-PAI-1 antibody of the disclosure) that is required for 50% inhibition of a given activity of the molecule the inhibitor targets (e.g., PAI-1). It will be understood by one of ordinary skill in the art that a lower IC50 value corresponds to a more potent inhibitor. When anti-PAI-1 antibodies inhibit the activity of human PAI-1 and PAI-1 from one or more additional species, inhibit can be with about the same IC50 value or substantially equipotent IC50 value.

In another embodiment, the anti-PAI-1 antibodies are described as not inhibiting one or more biological activities of PAI-1, such as binding of PAI-1 to vitronectin. The term “inhibition” as used herein, refers to any statistically significant decrease in biological activity, including full blocking of the activity. For example, “inhibition” can refer to a decrease of about 30%, 40%, 50%, 60%, 70%, 80%, 90%, or 100% in biological activity. In certain embodiments, the anti-PAI-1 antibodies does not inhibit one or more biological activities of PAI-1 by at least 10%, 20%, 30%, 40%, 50%, 60%, 70%, 80%, 90%, or 100%.

In one embodiment, the anti-PAI-1 antibodies of the disclosure exhibit a reduced antibody related toxicity as compared to an PAI-1 antibody that disrupt vitronectin binding. In another embodiment, the anti-PAI-1 antibodies of the disclosure exhibit toxicities that are at least 2 fold, or at least 3 fold, or at least 5 fold, or at least 7 fold, or a least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold, or are between 2 fold and 10 fold, or between 5 fold and 50 fold, or between 25 fold and 100 fold, or between 75 fold and 200 fold, or between 100 and 200 fold, less than that of an antibody that disrupts PAI-1:vitronectin binding. In another embodiment, the anti-PAI-1 antibodies of the disclosure exhibit toxicities that are reduced by at least 10%, or at least 20%, or by at least 30%, or by at least 40%, or by at least 50%, or by at least 60%, or by at least 70%, or by at least 80%, or by at least 90%, or by at least 100%, or by at least 200%, or by at least 300%, or by at least 400%, or by at least 500% relative to an antibody that disrupts PAI-1:vitronectin binding.

In certain embodiments, the antibodies of the disclosure are human, humanized, chimeric or antibody fragments.

In certain embodiments, the antibodies of the disclosure bind specifically to human PAI-1 and have one or more (1, 2, 3, 4, 5, 6, 7, 8, 9, 10, more than 10) of the Desired Characteristics.

In certain embodiments, the antibody or antibody fragment has at least any number of these characteristics, such as at least 3, 4, 5, 6, 7, 8, 9, 10, 11, 12, 13, 14, 15, 16. Of course, such antibodies may optionally have additional characteristics. It should be understood that antibodies defined based on possessing any one or more of the foregoing properties possesses, at least, such one or more properties but may also possess other functional or structural characteristics.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3, 4) of the following characteristics:

inhibits binding of human PAI-1 to uPA and tPA;

preferentially binds to active human PAI-1 over latent human PAI-1;

can reduce the level of VCAM-1 in dermal tissues;

can reduce the level of TNF-alpha in dermal tissues;

can stimulate plasmin-mediated activation of MMP-1.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3) of the following characteristics:

affinity (K_{D}) between 5 pm to 200 pM for active human PAI-1, as assessed by surface plasmon resonance;

immunospecifically binds to a human PAI-1:vitronectin complex, but does not disrupt the binding of human PAI-1 to vitronectin;

specifically binds to the reactive center loop of PAI-1 that interacts with tPA and uPA.

In certain embodiments, the antibody or antibody fragment has an affinity (K_{D}) of between about 5 pM and 125 pM for active human PAI-1, as assessed by surface plasmon resonance. In certain embodiments, the antibody or antibody fragment has an affinity (K_{D}) of less than or equal to 100 pM for active human PAI-1, as assessed by surface plasmon resonance. In certain embodiments, the antibody or antibody fragment has an affinity (K_{D}) of less than or equal to 50 pM for active human PAI-1, as assessed by surface plasmon resonance.

Such antibodies may have additional characteristics, including additional characteristics described in the examples.

In certain embodiments, the antibodies of the disclosure may bind epitopes conserved across species. In one embodiment, antibodies of the disclosure bind murine, non-human primate, rat, bovine, pig or other mammalian PAI-1 and antigenic fragments thereof. In one embodiment the antibodies of the disclosure may bind to one or more PAI-1 orthologs and or isoforms. In a specific embodiment, antibodies of the disclosure bind to PAI-1 and antigenic fragments thereof from one or more species, including, but not limited to, mouse, rat, monkey, primate, and human. In certain embodiments, the antibodies of the disclosure may bind an eptiope within humans across PAI-1 homologs and/or isoforms and/or conformational variants and/or subtypes.

The foregoing describes exemplary anti-PAI-1 antibodies for use in the claimed methods. Specific exemplary anti-PAI-1 antibodies are provided herein (See Examples and Tables 2, 16, and Sequence Listing). Functional characterstics of these specific exemplary antibodies are provided in the examples, and are also described throughout the application. It is contemplated that antibodies of the disclosure can be described using any one or more of the sequence (structural) and functional characteristics described herein, including features described in the examples and tables. Such antibodies can be used to inhibit fibrosis in a patient and/or to treat one or more diseases and conditions, such as those described herein.

### D. Production of Anti-PAI-1 Antibodies

The following describes exemplary techniques for the production of the antibodies useful in the present disclosure. Such techniques are merely exemplary. It should be understood that these techniques can be used in the production of antibodies and antibody fragments, where applicable. Throughout this section, reference to antibodies is intended to refer to both antibodies and, where applicable, antibody fragments. Moreover, the disclosure contemplates anti-PAI-1 antibodies and antibody fragments having any of the features (e.g., tagged, human, etc.) described in this section, as well as the use of such antibodies. Some of these techniques are described further in the Example section.

The PAI-1 antigen to be used for production of antibodies may be human PAI-1 or an antigenic fragment thereof. In particular embodiments, the antigenic fragment of human PAI-1 is a fragment that includes the reactive center loop that normally interacts with tPA/uPA. Human PAI-1 and PAI-1 fragments can be produced recombinantly in an isolated form from, bacterial or eukaryotic cells using standard recombinant DNA methodology. PAI-1 can be expressed as a tagged (e.g., epitope tag) or other fusion protein to facilitate isolation as well as identification in various assays. Antibodies or binding proteins that bind to various tags and fusion sequences are available as elaborated below. Other forms of PAI-1 useful for generating antibodies will be apparent to those skilled in the art.

### 1. Tags

Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. The FLAG-peptide [Hopp et al., BioTechnology, 6:1204-1210 (1988)] is recognized by an anti-FLAG M2 monoclonal antibody (Eastman Kodak Co., New Haven, Conn.). Purification of a protein containing the FLAG peptide can be performed by immunoaffinity chromatography using an affinity matrix comprising the anti-FLAG M2 monoclonal antibody covalently attached to agarose (Eastman Kodak Co., New Haven, Conn.). Other tag polypeptides include the KT3 epitope peptide [Martin et al., Science, 255:192-194 (1992)]; an α-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

### 2. Monoclonal Antibodies

Monoclonal antibodies can be prepared using a wide variety of techniques known in the art including the use of hybridoma (Kohler et al., Nature, 256:495 (1975); Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981), recombinant, and phage display technologies, or a combination thereof. The term “monoclonal antibody” as used herein refers to an antibody obtained from a population of substantially homogeneous or isolated antibodies, e.g., the individual antibodies comprising the population are identical except for possible naturally occurring mutations that may be present in minor amounts. Monoclonal antibodies are highly specific, being directed against a single antigenic site or multiple antigenic sites in the case of multispecific engineered antibodies. Furthermore, in contrast to polyclonal antibody preparations which include different antibodies directed against different determinants (epitopes), each monoclonal antibody is directed against the same determinant on the antigen. In addition to their specificity, the monoclonal antibodies are advantageous in that they may be synthesized uncontaminated by other antibodies. The modifier “monoclonal” is not to be construed as requiring production of the antibody by any particular method. Following is a description of representative methods for producing monoclonal antibodies which is not intended to be limiting and may be used to produce, for example, monoclonal mammalian, chimeric, humanized, human, domain, diabodies, vaccibodies, linear and multispecific antibodies.

### 3. Hybridoma Techniques

Methods for producing and screening for specific antibodies using hybridoma technology are routine and well known in the art. In the hybridoma method, mice or other appropriate host animals, such as hamster, are immunized to elicit lymphocytes that produce or are capable of producing antibodies that will specifically bind to the antigen used for immunization. Alternatively, lymphocytes may be immunized in vitro, as is typically done when using hybridoma technology to produce human monoclonal antibodies. After immunization (in vivo or in vitro), lymphocytes are isolated and then fused with a myeloma cell line using a suitable fusing agent or fusion partner, such as polyethylene glycol, to form a hybridoma cell (Goding, Monoclonal Antibodies: Principles and Practice, pp. 59-103 (Academic Press, 1986)). In certain embodiments, the selected myeloma cells are those that fuse efficiently, support stable high-level production of antibody by the selected antibody-producing cells, and are sensitive to a selective medium that selects against the unfused parental cells. In one aspect, the myeloma cell lines are murine myeloma lines, such as those derived from MOPC-21 and MPC-11 mouse tumors available from the Salk Institute Cell Distribution Center, San Diego, Calif. USA, and SP-2 and derivatives e.g., X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); and Brodeur et al., Monoclonal Antibody Production Techniques and Applications, pp. 51-63 (Marcel Dekker, Inc., New York, 1987)).

Once hybridoma cells that produce antibodies of the desired specificity, affinity, and/or activity are identified, the clones may be subcloned by limiting dilution procedures and grown by standard methods (Goding, Supra). Suitable culture media for this purpose include, for example, D-MEM or RPMI-1640 medium. In addition, the hybridoma cells may be grown in vivo as ascites tumors in an animal e.g, by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, affinity tags, hydroxylapatite chromatography, gel electrophoresis, dialysis, etc. Exemplary purification methods are described in more detail below.

### 4. Recombinant DNA Techniques

Methods for producing and screening for specific antibodies using recombinant DNA technology are routine and well known in the art (e.g. U.S. Pat. No. 4,816,567). DNA encoding the monoclonal antibodies may be readily isolated and/or sequenced using conventional procedures (e.g., by using oligonucleotide probes that are capable of binding specifically to genes encoding the heavy and light chains of murine antibodies). Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Pluckthun, Immunol. Revs., 130:151-188 (1992). As described below for antibodies generated by phage display and humanization of antibodies, DNA or genetic material for recombinant antibodies can be obtained from source(s) other than hybridomas to generate antibodies of the disclosure.

Recombinant expression of an antibody or variant thereof generally requires construction of an expression vector containing a polynucleotide that encodes the antibody. The disclosure, thus, provides replicable vectors comprising a nucleotide sequence encoding an antibody molecule, a heavy or light chain of an antibody, a heavy or light chain variable domain of an antibody or a portion thereof, or a heavy or light chain CDR, operably linked to a promoter. Such vectors may include the nucleotide sequence encoding the constant region of the antibody molecule (see, e.g., U.S. Pat. Nos. 5,981,216; 5,591,639; 5,658,759 and 5,122,464) and the variable domain of the antibody may be cloned into such a vector for expression of the entire heavy, the entire light chain, or both the entire heavy and light chains.

Once the expression vector is transferred to a host cell by conventional techniques, the transfected cells are then cultured by conventional techniques to produce an antibody. Thus, the disclosure includes host cells containing a polynucleotide encoding an antibody of the disclosure or fragments thereof, or a heavy or light chain thereof, or portion thereof, or a single-chain antibody of the disclosure, operably linked to a heterologous promoter. In certain embodiments for the expression of double-chained antibodies, vectors encoding both the heavy and light chains may be co-expressed in the host cell for expression of the entire immunoglobulin molecule, as detailed below.

Mammalian cell lines available as hosts for expression of recombinant antibodies are well known in the art and include many immortalized cell lines available from the American Type Culture Collection (ATCC), including but not limited to Chinese hamster ovary (CHO) cells, HeLa cells, baby hamster kidney (BHK) cells, monkey kidney cells (COS), human hepatocellular carcinoma cells (e.g., Hep G2), human epithelial kidney 293 cells, and a number of other cell lines. Different host cells have characteristic and specific mechanisms for the post-translational processing and modification of proteins and gene products. Appropriate cell lines or host systems can be chosen to ensure the correct modification and processing of the antibody or portion thereof expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT2O and T47D, NS0 (a murine myeloma cell line that does not endogenously produce any functional immunoglobulin chains), SP20, CRL7O3O and HsS78Bst cells. In one embodiment, human cell lines developed by immortalizing human lymphocytes can be used to recombinantly produce monoclonal antibodies. In one embodiment, the human cell line PER.C6. (Crucell, Netherlands) can be used to recombinantly produce monoclonal antibodies.

Additional cell lines which may be used as hosts for expression of recombinant antibodies include, but are not limited to, insect cells (e.g. Sf21/Sf9, Trichoplusia ni Bti-Tn5b1-4) or yeast cells (e.g. S. cerevisiae, Pichia, U.S. Pat. No. 7,326,681; etc), plants cells (US20080066200); and chicken cells (WO2008142124).

In certain embodiments, antibodies of the disclosure are expressed in a cell line with stable expression of the antibody. Stable expression can be used for long-term, high-yield production of recombinant proteins. For example, cell lines which stably express the antibody molecule may be generated. Host cells can be transformed with an appropriately engineered vector comprising expression control elements (e.g., promoter, enhancer, transcription terminators, polyadenylation sites, etc.), and a selectable marker gene. Following the introduction of the foreign DNA, cells may be allowed to grow for 1-2 days in an enriched media, and then are switched to a selective media. The selectable marker in the recombinant plasmid confers resistance to the selection and allows cells that stably integrated the plasmid into their chromosomes to grow and form foci which in turn can be cloned and expanded into cell lines. Methods for producing stable cell lines with a high yield are well known in the art and reagents are generally available commercially.

In certain embodiments, antibodies of the disclosure are expressed in a cell line with transient expression of the antibody. Transient transfection is a process in which the nucleic acid introduced into a cell does not integrate into the genome or chromosomal DNA of that cell. It is in fact maintained as an extrachromosomal element, e.g. as an episome, in the cell. Transcription processes of the nucleic acid of the episome are not affected and a protein encoded by the nucleic acid of the episome is produced.

The cell line, either stable or transiently transfected, is maintained in cell culture medium and conditions well known in the art resulting in the expression and production of monoclonal antibodies. In certain embodiments, the mammalian cell culture media is based on commercially available media formulations, including, for example, DMEM or Ham's F12. In other embodiments, the cell culture media is modified to support increases in both cell growth and biologic protein expression. As used herein, the terms “cell culture medium,” “culture medium,” and “medium formulation” refer to a nutritive solution for the maintenance, growth, propagation, or expansion of cells in an artificial in vitro environment outside of a multicellular organism or tissue. Cell culture medium may be optimized for a specific cell culture use, including, for example, cell culture growth medium which is formulated to promote cellular growth, or cell culture production medium which is formulated to promote recombinant protein production. The terms nutrient, ingredient, and component are used interchangeably herein to refer to the constituents that make up a cell culture medium.

In one embodiment, the cell lines are maintained using a fed batch method. As used herein, “fed batch method,” refers to a method by which a fed batch cell culture is supplied with additional nutrients after first being incubated with a basal medium. For example, a fed batch method may comprise adding supplemental media according to a determined feeding schedule within a given time period. Thus, a “fed batch cell culture” refers to a cell culture wherein the cells, typically mammalian, and culture medium are supplied to the culturing vessel initially and additional culture nutrients are fed, continuously or in discrete increments, to the culture during culturing, with or without periodic cell and/or product harvest before termination of culture.

The cell culture medium used and the nutrients contained therein are known to one of skill in the art. In one embodiment, the cell culture medium comprises a basal medium and at least one hydrolysate, e.g., soy-based, hydrolysate, a yeast-based hydrolysate, or a combination of the two types of hydrolysates resulting in a modified basal medium. In another embodiment, the additional nutrients may include only a basal medium, such as a concentrated basal medium, or may include only hydrolysates, or concentrated hydrolysates. Suitable basal media include, but are not limited to Dulbecco's Modified Eagle's Medium (DMEM), DME/F12, Minimal Essential Medium (MEM), Basal Medium Eagle (BME), RPMI 1640, F-10, F-12, α-Minimal Essential Medium (α-MEM), Glasgow's Minimal Essential Medium (G-MEM), PF CHO (see, e.g., CHO protein free medium (Sigma) or EX-CELL™ 325 PF CHO Serum-Free Medium for CHO Cells Protein-Free (SAFC Bioscience), and Iscove's Modified Dulbecco's Medium. Other examples of basal media which may be used in the disclosure include BME Basal Medium (Gibco-Invitrogen; see also Eagle, H (1965) Proc. Soc. Exp. Biol. Med. 89, 36); Dulbecco's Modified Eagle Medium (DMEM, powder) (Gibco-Invitrogen (#31600); see also Dulbecco and Freeman (1959) Virology 8, 396; Smith et al. (1960) Virology 12, 185. Tissue Culture Standards Committee, In Vitro 6:2, 93); CMRL 1066 Medium (Gibco-Invitrogen (#11530); see also Parker R. C. et al (1957) Special Publications, N.Y. Academy of Sciences, 5, 303).

In certain embodiments, the basal medium may be is serum-free, meaning that the medium contains no serum (e.g., fetal bovine serum (FBS), horse serum, goat serum, or any other animal-derived serum known to one skilled in the art) or animal protein free media or chemically defined media.

The basal medium may be modified in order to remove certain non-nutritional components found in standard basal medium, such as various inorganic and organic buffers, surfactant(s), and sodium chloride. Removing such components from basal cell medium allows an increased concentration of the remaining nutritional components, and may improve overall cell growth and protein expression. In addition, omitted components may be added back into the cell culture medium containing the modified basal cell medium according to the requirements of the cell culture conditions. In certain embodiments, the cell culture medium contains a modified basal cell medium, and at least one of the following nutrients, an iron source, a recombinant growth factor; a buffer; a surfactant; an osmolarity regulator; an energy source; and non-animal hydrolysates. In addition, the modified basal cell medium may optionally contain amino acids, vitamins, or a combination of both amino acids and vitamins. In another embodiment, the modified basal medium further contains glutamine, e.g, L-glutamine, and/or methotrexate.

In certain embodiments, antibody production is conducted in large quantity by a bioreactor process using fed-batch, batch, perfusion or continuous feed bioreactor methods known in the art. Large-scale bioreactors have at least 1000 liters of capacity, preferably about 1,000 to 100,000 liters of capacity. These bioreactors may use agitator impellers to distribute oxygen and nutrients. Small scale bioreactors refers generally to cell culturing in no more than approximately 100 liters in volumetric capacity, and can range from about 1 liter to about 100 liters. Alternatively, single-use bioreactors (SUB) may be used for either large-scale or small scale culturing.

Temperature, pH, agitation, aeration and inoculum density will vary depending upon the host cells used and the recombinant protein to be expressed. For example, a recombinant protein cell culture may be maintained at a temperature between 30 and 45 degrees Celsius. The pH of the culture medium may be monitored during the culture process such that the pH stays at an optimum level, which may be for certain host cells, within a pH range of 6.0 to 8.0. An impellor driven mixing may be used for such culture methods for agitation. The rotational speed of the impellor may be approximately 50 to 200 cm/sec tip speed, but other airlift or other mixing/aeration systems known in the art may be used, depending on the type of host cell being cultured. Sufficient aeration is provided to maintain a dissolved oxygen concentration of approximately 20% to 80% air saturation in the culture, again, depending upon the selected host cell being cultured. Alternatively, a bioreactor may sparge air or oxygen directly into the culture medium. Other methods of oxygen supply exist, including bubble-free aeration systems employing hollow fiber membrane aerators.

### 5. Phage Display Techniques

In another embodiment, monoclonal antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991). In such methods antibodies of the disclosure can be isolated by screening of a recombinant combinatorial antibody library, preferably a scFv phage display library, prepared using human VL and VH cDNAs prepared from mRNA derived from human lymphocytes. Methodologies for preparing and screening such libraries are known in the art. In addition to commercially available kits for generating phage display libraries (e.g., the Pharmacia Recombinant Phage Antibody System, catalog no. 27-9400-01; and the Stratagene SURFZAP™ phage display kit, catalog no. 240612), examples of methods and reagents particularly amenable for use in generating and screening antibody display libraries can be found in, for example, U.S. Pat. Nos. 6,248,516; 6,545,142; 6,291,158; 6,291,1591; 6,291,160; 6,291,161; 6,680,192; 5,969,108; 6,172,197; 6,806,079; 5,885,793; 6,521,404; 6,544,731; 6,555,313; 6,593,081; 6,582,915; 7,195,866. Thus, these techniques are viable alternatives to traditional monoclonal antibody hybridoma techniques for generation and isolation of monoclonal antibodies.

In phage display methods, functional antibody domains are displayed on the surface of phage particles which carry the polynucleotide sequences encoding them. In a particular embodiment, such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein.

As described in the above references, after phage selection, the antibody coding regions from the phage can be isolated and used to generate whole antibodies, including human antibodies, humanized antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in PCT publication WO 92/22324; Mullinax et al., BioTechniques 12(6):864-869 (1992); and Better et al., Science 240:1041-1043 (1988).

Examples of techniques which can be used to produce single-chain Fvs and antibodies include those described in U.S. Pat. Nos. 4,946,778 and 5,258,498. Thus, techniques described above and those well known in the art can be used to generate recombinant antibodies wherein the binding domain, e.g. ScFv, was isolated from a phage display library.

### 6. Antibody Purification and Isolation

Once an antibody molecule has been produced by recombinant or hybridoma expression, it may be purified by any method known in the art for purification of an immunoglobulin molecule, for example, by chromatography (e.g., ion exchange, affinity, particularly by affinity for the specific antigens Protein A or Protein G, and sizing column chromatography), centrifugation, differential solubility, or by any other standard technique for the purification of proteins. Further, the antibodies of the present disclosure or fragments thereof may be fused to heterologous polypeptide sequences (referred to herein as “tags”) described above or otherwise known in the art to facilitate purification.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology, 10:163-167 (1992) describe a procedure for isolating antibodies which are secreted into the periplasmic space of E. coli. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, hydrophobic interaction chromatography, ion exchange chromatography, gel electrophoresis, dialysis, and/or affinity chromatography either alone or in combination with other purification steps. The suitability of protein A as an affinity ligand depends on the species and isotype of any immunoglobulin Fc domain that is present in the antibody and will be understood by one of skill in the art. The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH₃ domain, the Bakerbond ABX resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin, SEPHAROSE chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, and performed at low salt concentrations (e.g., from about 0-0.25 M salt).

Thus, in certain embodiments is provided antibodies of the disclosure that are substantially purified/isolated. In one embodiment, these isolated/purified recombinantly expressed antibodies may be administered to a patient to mediate a prophylactic or therapeutic effect. In another embodiment these isolated/purified antibodies may be used to diagnose a PAI-1 mediated disease.

### 7. Humanized and Chimeric Antibodies

In certain embodiments, the antibodies and antibody fragments of the disclosure, including monoclonal antibodies, are humanized antibodies, which are generated using methods well known in the art. Humanized antibodies are antibody molecules derived from a non-human species antibody (also referred to herein as a donor antibody) that bind the desired antigen having one or more complementarity determining regions (CDRs) from the non-human species and a framework region from a human immunoglobulin molecule (also referred to herein as an acceptor antibody). Often, framework residues in the human framework regions will be substituted with the corresponding residue from the CDR donor antibody to alter, preferably improve, antigen binding and/or reduce immunogenicity. These framework substitutions are identified by methods well known in the art, e.g., by modeling of the interactions of the CDR and framework residues to identify framework residues important for antigen binding and sequence comparison to identify unusual framework residues at particular positions. (See, e.g, Riechmann et al., Nature 332:323 (1988)). In practice, and in certain embodiments, humanized antibodies are typically human antibodies in which some hypervariable region residues and possibly some FR residues are substituted by residues from analogous sites in rodent antibodies. In alterntive embodiments, the FR residues are fully human residues.

Humanization can be essentially performed following the method of Winter and co-workers (Jones et al., Nature, 321:522-525 (1986); Reichmann et al., Supra; Verhoeyen et al., Science, 239:1534-1536 (1988)), by substituting hypervariable region sequences for the corresponding sequences of a human antibody. Specifically, humanized antibodies may be prepared by methods well known in the art including CDR grafting approaches (see, e.g., U.S. Pat. No. 6,548,640), veneering or resurfacing (U.S. Pat. Nos. 5,639,641 and 6,797,492; Studnicka et al., Protein Engineering 7(6):805-814 (1994); Roguska. et al., PNAS 91:969-973 (1994)), chain shuffling strategies (see e.g., U.S. Pat. No. 5,565,332; Rader et al., Proc. Natl. Acad. Sci. USA (1998) 95:8910-8915), molecular modeling strategies (U.S. Pat. No. 5,639,641), and the like. These general approaches may be combined with standard mutagenesis and recombinant synthesis techniques to produce anti-PAI-1 antibodies with desired properties.

CDR grafting is performed by replacing one or more CDRs of an acceptor antibody (e.g., a human antibody) with one or more CDRs of a donor antibody (e.g., a non-human antibody). Acceptor antibodies may be selected based on similarity of framework residues between a candidate acceptor antibody and a donor antibody and may be further modified to introduce similar residues. Following CDR grafting, additional changes may be made in the donor and/or acceptor sequences to optimize antibody binding and functionality.

Grafting of abbreviated CDR regions is a related approach. Abbreviated CDR regions include the specificity-determining residues and adjacent amino acids, including those at positions 27d-34, 50-55 and 89-96 in the light chain, and at positions 31-35b, 50-58, and 95-101 in the heavy chain. See (Padlan et al. (1995) FASEB J. 9: 133-9). Grafting of specificity-determining residues (SDRs) is premised on the understanding that the binding specificity and affinity of an antibody combining site is determined by the most highly variable residues within each of the CDR regions. Analysis of the three-dimensional structures of antibody-antigen complexes, combined with analysis of the available amino acid sequence data was used to model sequence variability based on structural dissimilarity of amino acid residues that occur at each position within the CDR. Minimally immunogenic polypeptide sequences consisting of contact residues, which are referred to as SDRs, are identified and grafted onto human framework regions.

Veneering or resurfacing is based on the concept of reducing potentially immunogenic amino acid sequences in a rodent or other non-human antibody by resurfacing the solvent accessible exterior of the antibody with human amino acid sequences. Thus, veneered antibodies appear less foreign to human cells. A non-human antibody is veneered by (1) identifying exposed exterior framework region residues in the non-human antibody, which are different from those at the same positions in framework regions of a human antibody, and (2) replacing the identified residues with amino acids that typically occupy these same positions in human antibodies.

By definition, humanized antibodies are chimeric antibodies. Chimeric antibodies are antibodies in which a portion of the heavy and/or light chain is identical with or homologous to corresponding sequences in antibodies derived from a particular species or belonging to a particular antibody class or subclass, while another portion of the chain(s) is identical with or homologous to corresponding sequences in antibodies derived from another species or belonging to another antibody class or subclass, as well as fragments of such antibodies, so long as they exhibit the desired biological activity (e.g., Morrison et al., Proc. Natl. Acad. Sci. USA, 81:6851-6855 (1984)). Chimeric antibodies of interest herein include “primatized” antibodies comprising variable domain antigen-binding sequences derived from a nonhuman primate (e.g., Old World Monkey, such as baboon, rhesus or cynomolgus monkey) and human constant region sequences (U.S. Pat. No. 5,693,780).

### 8. Human Antibodies

As an alternative to humanization, human antibodies can be generated using methods well known in the art. Thus, in certain embodiments, the antibodies and antibody fragments, including monoclonal antibodies, are human antibodies. Human antibodies avoid some of the problems associated with antibodies that possess murine or rat variable and/or constant regions. The presence of such murine or rat derived proteins can lead to the rapid clearance of the antibodies or can lead to the generation of an immune response against the antibody by a patient. In order to avoid the utilization of murine or rat derived antibodies, fully human antibodies can be generated through the introduction of functional human antibody loci into a rodent, other mammal or animal so that the rodent, other mammal or animal produces fully human antibodies.

For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggemann et al., Year in Immuno., 7:33 (1993); U.S. Pat. Nos. 5,545,806, 5,569,825, 5,591,669 (all of GenPharm); U.S. Pat. No. 5,545,807; and WO 97/17852. In practice, the use of XENOMOUSE® strains of mice that have been engineered to contain up to but less than 1000 kb-sized germline configured fragments of the human heavy chain locus and kappa light chain locus. See Mendez et al. Nature Genetics 15:146-156 (1997) and Green and Jakobovits J. Exp. Med. 188:483-495 (1998). The XENOMOUSE® strains are available from Amgen, Inc. (Fremont, Calif.).

The production of the XENOMOUSE® strains of mice and antibodies produced in those mice is further discussed and delineated in U.S. Pat. Nos. 6,673,986; 7,049,426; 6,833,268; 6,162,963, 6,150,584, 6,114,598, 6,075,181, 6,657,103; 6,713,610 and 5,939,598; US Publication Nos. 2004/0010810; 2003/0229905; 2004/0093622; 2005/0054055; 2005/0076395; and 2006/0040363.

Essentially, XENOMOUSE® lines of mice are immunized with an antigen of interest (e.g. PAI-1), lymphatic cells (such as B-cells) are recovered from the hyper-immunized mice, and the recovered lymphocytes are fused with a myeloid-type cell line to prepare immortal hybridoma cell lines using techniques described above an well known in the art. These hybridoma cell lines are screened and selected to identify hybridoma cell lines that produced antibodies specific to the antigen of interest.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more V_{H} genes, one or more D_{H} genes, one or more J_{H} genes, a mu constant region, and usually a second constant region (preferably a gamma constant region) are formed into a construct for insertion into an animal. This approach is described in U.S. Pat. Nos. 5,545,807; 5,545,806; 5,625,825; 5,625,126; 5,633,425; 5,661,016; 5,770,429; 5,789,650; 5,814,318; 5,877,397; 5,874,299; 6,255,458; 5,591,669; 6,023,010; 5,612,205; 5,721,367; 5,789,215; 5,643,763; and 5,981,175.

Kirin has also demonstrated the generation of human antibodies from mice in which, through microcell fusion, large pieces of chromosomes, or entire chromosomes, have been introduced. See U.S. Pat. No. 6,632,976. Additionally, KM™-mice, which are the result of cross-breeding of Kirin's Tc mice with Medarex's minilocus (Humab) mice have been generated. These mice possess the human IgH transchromosome of the Kirin mice and the kappa chain transgene of the Genpharm mice (Ishida et al., Cloning Stem Cells, (2002) 4:91-102).

Human antibodies can also be derived by in vitro methods. Suitable examples include but are not limited to phage display (MedImmune (formerly CAT), Morphosys, Dyax, Biosite/Medarex, Xoma, Symphogen, Alexion (formerly Proliferon), Affimed) ribosome display (MedImmune (formerly CAT)), yeast display, and the like. The phage display technology (See e.g., U.S. Pat. No. 5,969,108) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S, and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

As discussed above, human antibodies may also be generated by in vitro activated B cells (see U.S. Pat. Nos. 5,567,610 and 5,229,275).

Immunoglobulin genes undergo various modifications during maturation of the immune response, including recombination between V, D and J gene segments, isotype switching, and hypermutation in the variable regions. Recombination and somatic hypermutation are the foundation for generation of antibody diversity and affinity maturation, but they can also generate sequence liabilities that may make commercial production of such immunoglobulins as therapeutic agents difficult or increase the immunogenicity risk of the antibody. In general, mutations in CDR regions are likely to contribute to improved affinity and function, while mutations in framework regions may increase the risk of immunogenicity. This risk can be reduced by reverting framework mutations to germline while ensuring that activity of the antibody is not adversely impacted. The diversification processes may also generate some structural liabilities or these structural liabilities may exist within germline sequences contributing to the heavy and light chain variable domains. Regardless of the source, it may be desirable to remove potential structural liabilities that may result in instability, aggregation, heterogeneity of product, or increased immunogenicity. Examples of undesirable liabilities include unpaired cysteines (which may lead to disulfide bond scrambling, or variable sulfhydryl adduct formation), N-linked glycosylation sites (resulting in heterogeneity of structure and activity), as well as deamidation (e.g. NG, NS), isomerization (DG), oxidation (exposed methionine), and hydrolysis (DP) sites.

Accordingly, in order to reduce the risk of immunogenicity and improve pharmaceutical properties of the antibodies of the disclosure, it may be desirable to revert a framework sequence to germline, revert a CDR to germline, and/or remove a structural liability.

### 9. Antibody Fragments

In certain embodiments, the present antibodies are antibody fragments or antibodies comprising these fragments. The antibody fragment comprises a portion of the full length antibody, which generally is the antigen binding or variable region thereof. Examples of antibody fragments include Fab, Fab′, F(ab′)₂, Fd and Fv fragments. Diabodies; linear antibodies (U.S. Pat. No. 5,641,870); single-chain antibody molecules; and multispecific antibodies are antibodies formed from these antibody fragments.

Traditionally, these fragments were derived via proteolytic digestion of intact antibodies using techniques well known in the art. However, these fragments can now be produced directly by recombinant host cells. Fab, Fv and scFv antibody fragments can all be expressed in and secreted from E. coli, thus allowing the facile production of large amounts of these fragments. In one embodiment, the antibody fragments can be isolated from the antibody phage libraries discussed above. Alternatively, Fab′-SH fragments can also be directly recovered from E. coli and chemically coupled to form F(ab′)₂ fragments (Carter et al., Bio/Technology, 10:163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single-chain Fv fragment (scFv). In certain embodiments, the antibody is not a Fab fragment. Fv and scFv are the only species with intact combining sites that are devoid of constant regions; thus, they are suitable for reduced nonspecific binding during in vivo use. scFv fusion proteins may be constructed to yield fusion of an effector protein at either the amino or the carboxy terminus of an scFv.

In certain embodiments, the present antibodies are domain antibodies, e.g., antibodies containing the small functional binding units of antibodies, corresponding to the variable regions of the heavy (V_{H}) or light (V_{L}) chains of human antibodies. Examples of domain antibodies include, but are not limited to, those available from Domantis that are specific to therapeutic targets (see, for example, WO04/058821; WO04/081026; WO04/003019; WO03/002609; U.S. Pat. Nos. 6,291,158; 6,582,915; 6,696,245; and 6,593,081).

In certain embodiments of the disclosure, the present antibodies are linear antibodies. Linear antibodies comprise a pair of tandem Fd segments (V_{H}-C_{H1}-V_{H}-C_{H1}) which form a pair of antigen-binding regions. Linear antibodies can be bispecific or monospecific. See, Zapata et al., Protein Eng., 8(10):1057-1062 (1995).

### 10. Bispecific Antibodies

Bispecific antibodies are antibodies that have binding specificities for at least two different epitopes. Exemplary bispecific antibodies may bind to two different epitopes of the PAI-1 protein. Other such antibodies may combine a PAI-1 binding site with a binding site for another protein. Alternatively, an anti-PAI-1 arm may be combined with an arm which binds to a triggering molecule on a leukocyte such as a T-cell receptor molecule (e.g. CD3), or Fc receptors for IgG (FcγR), such as FcγRI (CD64), FcγRII (CD32) and FcγRIII (CD16), so as to focus and localize cellular defense mechanisms to the PAI-1-expressing cell. Bispecific antibodies may also be used to localize cytotoxic agents to cells which express PAI-1. These antibodies possess a PAI-1-binding arm and an arm which binds the cytotoxic agent (e.g. saporin, anti-interferon-α, vinca alkaloid, ricin A chain, methotrexate or radioactive isotope hapten). Bispecific antibodies can be prepared as full length antibodies or antibody fragments (e.g. F(ab′)2 bispecific antibodies). Methods for making bispecific antibodies are known in the art. (See, for example, Millstein et al., Nature, 305:537-539 (1983); Traunecker et al., EMBO J., 10:3655-3659 (1991); Suresh et al., Methods in Enzymology, 121:210 (1986); Kostelny et al., J. Immunol., 148(5):1547-1553 (1992); Hollinger et al., Proc. Natl. Acad. Sci. USA, 90:6444-6448 (1993); Gruber et al., J. Immunol., 152:5368 (1994); U.S. Pat. Nos. 4,474,893; 4,714,681; 4,925,648; 5,573,920; 5,601,819; 5,731,168; 4,676,980; 5,897,861; 5,660,827; 5,811,267; 5,849,877; 5,948,647; 5,959,084; 6,106,833; 6,143,873 and 4,676,980, WO 94/04690; and WO 92/20373.)

Traditional production of full length bispecific antibodies is based on the co-expression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fused to immunoglobulin constant domain sequences. Preferably, the fusion is with an Ig heavy chain constant domain, comprising at least part of the hinge, C_{H}2, and C_{H}3 regions. It is preferred to have the first heavy-chain constant region (C_{H}1) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant affect on the yield of the desired chain combination.

In one embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure may facilitate the separation of the desired bispecific compound from unwanted immunoglobulin chain combinations, as the presence of an immunoglobulin light chain in only one half of the bispecific molecule provides for a facile way of separation. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers which are recovered from recombinant cell culture. The preferred interface comprises at least a part of the C_{H}3 domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (U.S. Pat. No. 5,897,861). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med., 175: 217-225 (1992) describe the production of a fully humanized bispecific antibody F(ab′)2 molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci USA, 90:6444-6448 (1993) has provided an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (sFv) dimers has also been reported. See Gruber et al., J. Immunol., 152:5368 (1994) and U.S. Pat. Nos. 5,591,828; 4,946,778; 5,455,030; and 5,869,620.

Antibodies with more than two valencies are contemplated. For example, trispecific antibodies can be prepared, Tutt et al. J. Immunol. 147: 60 (1991), and multispecific valencies U.S. Pat. No. 5,258,498.

### 11. Other Amino Acid Sequence Modifications

In addition to the above described human, humanized and/or chimeric antibodies, the present disclosure also encompasses further modifications and, their variants and fragments thereof, of the anti-PAI-1 antibodies of the disclosure comprising one or more amino acid residues and/or polypeptide substitutions, additions and/or deletions in the variable light (V_{L}) domain and/or variable heavy (V_{H}) domain and/or Fc region and post translational modifications. Included in these modifications are antibody conjugates wherein an antibody has been covalently attached to a moiety. Moieties suitable for attachment to the antibodies include but are not limited to, proteins, peptides, drugs, labels, and cytotoxins. These changes to the antibodies may be made to alter or fine tune the characteristics (biochemical, binding and/or functional) of the antibodies as is appropriate for treatment and/or diagnosis of PAI-1 mediated diseases. Methods for forming conjugates, making amino acid and/or polypeptide changes and post-translational modifications are well known in the art, some of which are detailed below. The following description is not intended to be limiting, but instead a non-limiting description of some embodiments, more of which will be obvious to one of skill in the art. It is also understood that some of the following methods were used to develop the human, humanized and/or chimeric antibody sequences described above. Any combination of deletion, insertion, and substitution is made to arrive at the final construct, provided that the final construct possesses the desired characteristics.

Amino acid changes to the antibodies necessarily results in sequences that are less than 100% identical to the above identified antibody sequences or parent antibody sequence. In certain embodiments, in this context, the antibodies many have about 25% to about 95% sequence identity to the amino acid sequence of either the heavy or light chain variable domain of an anti-PAI-1 antibody as described herein. Thus, in one embodiment a modified antibody may have an amino acid sequence having at least 25%, 35%, 45%, 55%, 65%, 75%, 80%, 85%, 90%, or 95% amino acid sequence identity or similarity with the amino acid sequence of either the heavy or light chain variable domain of an anti-PAI-1 antibody as described herein. In another embodiment, an altered antibody may have an amino acid sequence having at least 25%, 35%, 45%, 55%, 65%, 75%, 80%, 85%, 90%, or 95% amino acid sequence identity or similarity with the amino acid sequence of the heavy or light chain CDR1, CDR2, or CDR3 of an anti-PAI-1 antibody as described herein. In another embodiment, an altered antibody may have an amino acid sequence having at least 25%, 35%, 45%, 55%, 65%, 75%, 80%, 85%, 90%, or 95% amino acid sequence identity or similarity with the amino acid sequence of the heavy or light chain FR1, FR2, FR3 or FR4 of an anti-PAI-1 antibody as described herein.

In certain embodiments, altered antibodies are generated by one or more amino acid alterations (e.g., substitutions, deletion and/or additions) introduced in one or more of the variable regions of the antibody. In another embodiment, the amino acid alterations are introduced in the framework regions. One or more alterations of framework region residues may result in an improvement in the binding affinity of the antibody for the antigen. This may be especially true when these changes are made to humanized antibodies wherein the framework region may be from a different species than the CDR regions. Examples of framework region residues to modify include those which non-covalently bind antigen directly (Amit et al., Science, 233:747-753 (1986)); interact with/effect the conformation of a CDR (Chothia et al., J. Mol. Biol., 196:901-917 (1987)); and/or participate in the V_{L}-V_{H} interface (U.S. Pat. Nos. 5,225,539 and 6,548,640). In one embodiment, from about one to about five framework residues may be altered. Sometimes, this may be sufficient to yield an antibody mutant suitable for use in preclinical trials, even where none of the hypervariable region residues have been altered. Normally, however, an altered antibody will comprise additional hypervariable region alteration(s). In certain embodiments, the hypervariable region residues may be changed randomly, especially where the starting binding affinity of an anti-PAI-1 antibody for the antigen from the second mammalian species is such that such randomly produced antibodies can be readily screened.

One useful procedure for generating altered antibodies is called “alanine scanning mutagenesis” (Cunningham and Wells, Science, 244:1081-1085 (1989)). In this method, one or more of the hypervariable region residue(s) are replaced by alanine or polyalanine residue(s) to alter the interaction of the amino acids with the PAI-1. Those hypervariable region residue(s) demonstrating functional sensitivity to the substitutions then are refined by introducing additional or other mutations at or for the sites of substitution. Thus, while the site for introducing an amino acid sequence variation is predetermined, the nature of the mutation per se need not be predetermined. The Ala-mutants produced this way are screened for their biological activity as described herein.

In certain embodiments the substitutional variant involves substituting one or more hypervariable region residues of a parent antibody (e.g. a humanized or human antibody). Generally, the resulting variant(s) selected for further development will have improved biological properties relative to the parent antibody from which they are generated. A convenient way for generating such substitutional variants involves affinity maturation using phage display (Hawkins et al., J. Mol. Biol., 254:889-896 (1992) and Lowman et al., Biochemistry, 30(45):10832-10837 (1991)). Briefly, several hypervariable region sites (e.g., 6-7 sites) are mutated to generate all possible amino acid substitutions at each site. The antibody mutants thus generated are displayed in a monovalent fashion from filamentous phage particles as fusions to the gene III product of M13 packaged within each particle. The phage-displayed mutants are then screened for their biological activity (e.g., binding affinity) as herein disclosed.

Mutations in antibody sequences may include substitutions, deletions, including internal deletions, additions, including additions yielding fusion proteins, or conservative substitutions of amino acid residues within and/or adjacent to the amino acid sequence, but that result in a “silent” change, in that the change produces a functionally equivalent anti-PAI-1 antibody. Conservative amino acid substitutions may be made on the basis of similarity in polarity, charge, solubility, hydrophobicity, hydrophilicity, and/or the amphipathic nature of the residues involved. For example, non-polar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. In addition, glycine and proline are residues that can influence chain orientation. Non-conservative substitutions will entail exchanging a member of one of these classes for a member of another class. Furthermore, if desired, non-classical amino acids or chemical amino acid analogs can be introduced as a substitution or addition into the antibody sequence. Non-classical amino acids include, but are not limited to, the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general.

In another embodiment, any cysteine residue not involved in maintaining the proper conformation of the anti-PAI-1 antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

In certain embodiments of the disclosure, an antibody can be modified to produce fusion proteins; i.e., the antibody, or a fragment thereof, fused to a heterologous protein, polypeptide or peptide. In certain embodiments, the protein fused to the portion of an antibody is an enzyme component of Antibody-Directed Enzyme Prodrug Therapy (ADEPT). Examples of other proteins or polypeptides that can be engineered as a fusion protein with an antibody include, but are not limited to toxins such as ricin, abrin, ribonuclease, DNase I, Staphylococcal enterotoxin-A, pokeweed anti-viral protein, gelonin, diphtherin toxin, Pseudomonas exotoxin, and Pseudomonas endotoxin. See, for example, Pastan et al., Cell, 47:641 (1986), and Goldenberg et al., Cancer Journal for Clinicians, 44:43 (1994). Enzymatically active toxins and fragments thereof which can be used include diphtheria A chain, non-binding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232.

Additional fusion proteins may be generated through the techniques of gene-shuffling, motif-shuffling, exon-shuffling, and/or codon-shuffling (collectively referred to as “DNA shuffling”). DNA shuffling may be employed to alter the characteristics of the antibody or fragments thereof (e.g., an antibody or a fragment thereof with higher affinities and lower dissociation rates). See, generally, U.S. Pat. Nos. 5,605,793; 5,811,238; 5,830,721; 5,834,252; and 5,837,458, and Patten et al., 1997, Curr. Opinion Biotechnol., 8:724-33; Harayama, 1998, Trends Biotechnol. 16(2):76-82; Hansson et al., 1999, J. Mol. Biol., 287:265-76; and Lorenzo and Blasco, 1998, Biotechniques 24(2):308-313. The antibody can further be a binding-domain immunoglobulin fusion protein as described in U.S. Publication 2003/0118592, and PCT Publication WO 02/056910.

### 12. Variant Fc Regions

It is known that variants of the Fc region (e.g., amino acid substitutions and/or additions and/or deletions) enhance or diminish effector function of the antibody (See e.g., U.S. Pat. Nos. 5,624,821; 5,885,573; 6,538,124; 7,317,091; 5,648,260; 6,538,124; WO 03/074679; WO 04/029207; WO 04/099249; WO 99/58572; US Publication No. 2006/0134105; 2004/0132101; 2006/0008883) and may alter the pharmacokinetic properties (e.g. half-life) of the antibody (see, U.S. Pat. Nos. 6,277,375 and 7,083,784). Thus, in certain embodiments, the anti-PAI-1 antibodies of the disclosure comprise an altered Fc region (also referred to herein as “variant Fc region”) in which one or more alterations have been made in the Fc region in order to change functional and/or pharmacokinetic properties of the antibodies. Such alterations may result in a decrease or increase of Clq binding and complement dependent cytotoxicity (CDC) or of FcγR binding, for IgG, and antibody-dependent cellular cytotoxicity (ADCC), or antibody dependent cell-mediated phagocytosis (ADCP). The present disclosure encompasses the antibodies described herein with variant Fc regions wherein changes have been made to fine tune the effector function, enhancing or diminishing, providing a desired effector function. Accordingly, in one embodiment of the disclosure, the anti-PAI-1 antibodies of the disclosure comprise a variant Fc region (i.e., Fc regions that have been altered as discussed below). Anti-PAI-1 antibodies of the disclosure comprising a variant Fc region are also referred to here as “Fc variant antibodies.” As used herein native refers to the unmodified parental sequence and the antibody comprising a native Fc region is herein referred to as a “native Fc antibody”. Fc variant antibodies can be generated by numerous methods well known to one skilled in the art. Non-limiting examples include, isolating antibody coding regions (e.g., from hybridoma) and making one or more desired substitutions in the Fc region of the isolated antibody coding region. Alternatively, the antigent-binding portion (e.g., variable regions) of an anti-PAI-1 antibody may be subcloned into a vector encoding a variant Fc region. In one embodiment, the variant Fc region exhibits a similar level of inducing effector function as compared to the native Fc region. In another embodiment, the variant Fc region exhibits a higher induction of effector function as compared to the native Fc. In another embodiment, the variant Fc region exhibits lower induction of effector function as compared to the native Fc. Some specific embodiments of variant Fc regions are detailed infra. Methods for measuring effector function are well known in the art.

The effector function of an antibody is modified through changes in the Fc region, including but not limited to, amino acid substitutions, amino acid additions, amino acid deletions and changes in post translational modifications to Fc amino acids (e.g. glycosylation). The methods described below may be used to fine tune the effector function of a present antibody, a ratio of the binding properties of the Fc region for the FcR (e.g., affinity and specificity), resulting in a therapeutic antibody with the desired properties for a particular disease indication and taking into consideration the biology of PAI-1.

It is understood that the Fc region as used herein includes the polypeptides comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cgamma2 and Cgamma3 (Cγ2 and Cγ3) and the hinge between Cgamma1 (Cγ1) and Cgamma2 (Cγ2). Although the boundaries of the Fc region may vary, the human IgG heavy chain Fc region is usually defined to comprise residues C226 or P230 to its carboxyl-terminus, wherein the numbering is according to the EU index as set forth in Kabat. Fc may refer to this region in isolation, or this region in the context of an antibody, antibody fragment, or Fc fusion protein. Polymorphisms have been observed at a number of different Fc positions, including but not limited to positions 270, 272, 312, 315, 356, and 358 as numbered by the EU index, and thus slight differences between the presented sequence and sequences in the prior art may exist.

In one embodiment, the present disclosure encompasses Fc variant antibodies which have altered binding properties for an Fc ligand (e.g., an Fc receptor, Clq) relative to a native Fc antibody. Examples of binding properties include but are not limited to, binding specificity, equilibrium dissociation constant (Kd), dissociation and association rates (koff and kon respectively), binding affinity and/or avidity. It is known in the art that the equilibrium dissociation constant (Kd) is defined as koff/kon. In certain aspects, an antibody comprising an Fc variant region with a low Kd may be more desirable to an antibody with a high Kd. However, in some instances the value of the kon or koff may be more relevant than the value of the Kd. One skilled in the art can determine which kinetic parameter is most important for a given antibody application. For example, a modification that reduces binding to one or more positive regulator (e.g., FcγRIIIA) and/or enhanced binding to an inhibitory Fc receptor (e.g., FcγRIIB) would be suitable for reducing ADCC activity. Accordingly, the ratio of binding affinities (e.g., the ratio of equilibrium dissociation constants (Kd)) for different receptors can indicate if the ADCC activity of an Fc variant antibody of the disclosure is enhanced or decreased. Additionally, a modification that reduces binding to Clq would be suitable for reducing or eliminating CDC activity.

In one embodiment, Fc variant antibodies exhibit altered binding affinity for one or more Fc receptors including, but not limited to FcRn, FcγRI (CD64) including isoforms FcγRIA, FcγRIB, and FcγRIC; FcγRII (CD32 including isoforms FcγRIIA, FcγRIIB, and FcγRIIC); and FcγRII (CD16, including isoforms FcγRIIIA and FcγRIIIB) as compared to an native Fc antibody.

In one embodiment, an Fc variant antibody has enhanced binding to one or more Fc ligand relative to a native Fc antibody. In another embodiment, the Fc variant antibody exhibits increased or decreased affinity for an Fc ligand that is at least 2 fold, or at least 3 fold, or at least 5 fold, or at least 7 fold, or a least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold, or is between 2 fold and 10 fold, or between 5 fold and 50 fold, or between 25 fold and 100 fold, or between 75 fold and 200 fold, or between 100 and 200 fold, more or less than a native Fc antibody. In another embodiment, Fc variant antibodies exhibit affinities for an Fc ligand that are at least 90%, at least 80%, at least 70%, at least 60%, at least 50%, at least 40%, at least 30%, at least 20%, at least 10%, or at least 5% more or less than an native Fc antibody. In certain embodiments, an Fc variant antibody has increased affinity for an Fc ligand. In other embodiments, an Fc variant antibody has decreased affinity for an Fc ligand.

In a specific embodiment, an Fc variant antibody has enhanced binding to the Fc receptor FcγRIIIA In another specific embodiment, an Fc variant antibody has enhanced binding to the Fc receptor FcγRIIB In a further specific embodiment, an Fc variant antibody has enhanced binding to both the Fc receptors FcγRIIIA and FcγRIIB In certain embodiments, Fc variant antibodies that have enhanced binding to FcγRIIIA do not have a concomitant increase in binding the FcγRIIB receptor as compared to a native Fc antibody. In a specific embodiment, an Fc variant antibody has reduced binding to the Fc receptor FcγRIIIA In a further specific embodiment, an Fc variant antibody has reduced binding to the Fc receptor FcγRIIB In still another specific embodiment, an Fc variant antibody exhibiting altered affinity for FcγRIIIA and/or FcγRIIB has enhanced binding to the Fc receptor FcRn. In yet another specific embodiment, an Fc variant antibody exhibiting altered affinity for FcγRIIIA and/or FcγRIIB has altered binding to C1q relative to a native Fc antibody.

In one embodiment, Fc variant antibodies exhibit affinities for FcγRIIIA receptor that are at least 2 fold, or at least 3 fold, or at least 5 fold, or at least 7 fold, or a least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold, or are between 2 fold and 10 fold, or between 5 fold and 50 fold, or between 25 fold and 100 fold, or between 75 fold and 200 fold, or between 100 and 200 fold, more or less than an native Fc antibody. In another embodiment, Fc variant antibodies exhibit affinities for FcγRIIIA that are at least 90%, at least 80%, at least 70%, at least 60%, at least 50%, at least 40%, at least 30%, at least 20%, at least 10%, or at least 5% more or less than an native Fc antibody.

In one embodiment, Fc variant antibodies exhibit affinities for FcγRIIB receptor that are at least 2 fold, or at least 3 fold, or at least 5 fold, or at least 7 fold, or a least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold, or are between 2 fold and 10 fold, or between 5 fold and 50 fold, or between 25 fold and 100 fold, or between 75 fold and 200 fold, or between 100 and 200 fold, more or less than an native Fc antibody. In another embodiment, Fc variant antibodies exhibit affinities for FcγRIIB that are at least 90%, at least 80%, at least 70%, at least 60%, at least 50%, at least 40%, at least 30%, at least 20%, at least 10%, or at least 5% more or less than an native Fc antibody.

In one embodiment, Fc variant antibodies exhibit increased or decreased affinities to C1q relative to a native Fc antibody. In another embodiment, Fc variant antibodies exhibit affinities for C1q receptor that are at least 2 fold, or at least 3 fold, or at least 5 fold, or at least 7 fold, or a least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold, or are between 2 fold and 10 fold, or between 5 fold and 50 fold, or between 25 fold and 100 fold, or between 75 fold and 200 fold, or between 100 and 200 fold, more or less than an native Fc antibody. In another embodiment, Fc variant antibodies exhibit affinities for C1q that are at least 90%, at least 80%, at least 70%, at least 60%, at least 50%, at least 40%, at least 30%, at least 20%, at least 10%, or at least 5% more or less than an native Fc antibody. In still another specific embodiment, an Fc variant antibody exhibiting altered affinity for Ciq has enhanced binding to the Fc receptor FcRn. In yet another specific embodiment, an Fc variant antibody exhibiting altered affinity for C1q has altered binding to FcγRIIIA and/or FcγRIIB relative to a native Fc antibody.

It is well known in the art that antibodies are capable of directing the attack and destruction of targeted antigen through multiple processes collectively known in the art as antibody effector functions. One of these processes, known as “antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enables these cytotoxic effector cells to bind specifically to an antigen-bearing target cell and subsequently kill the target cell with cytotoxins. Specific high-affinity IgG antibodies directed to the surface of target cells “arm” the cytotoxic cells and are required for such killing. Lysis of the target cell is extracellular, requires direct cell-to-cell contact, and does not involve complement. Another process encompassed by the term effector function is complement dependent cytotoxicity (hereinafter referred to as “CDC”) which refers to a biochemical event of antibody-mediated target cell destruction by the complement system. The complement system is a complex system of proteins found in normal blood plasma that combines with antibodies to destroy pathogenic bacteria and other foreign cells. Still another process encompassed by the term effector function is antibody dependent cell-mediated phagocytosis (ADCP) which refers to a cell-mediated reaction wherein nonspecific cytotoxic cells that express one or more effector ligands recognize bound antibody on a target cell and subsequently cause phagocytosis of the target cell.

It is contemplated that Fc variant antibodies are characterized by in vitro functional assays for determining one or more FcγR mediated effector cell functions. In certain embodiments, Fc variant antibodies have similar binding properties and effector cell functions in in vivo models (such as those described and disclosed herein) as those in in vitro based assays. However, the present disclosure does not exclude Fc variant antibodies that do not exhibit the desired phenotype in in vitro based assays but do exhibit the desired phenotype in vivo.

The serum half-life of proteins comprising Fc regions may be increased by increasing the binding affinity of the Fc region for FcRn. The term “antibody half-life” as used herein means a pharmacokinetic property of an antibody that is a measure of the mean survival time of antibody molecules following their administration. Antibody half-life can be expressed as the time required to eliminate 50 percent of a known quantity of immunoglobulin from the patient's body (or other mammal) or a specific compartment thereof, for example, as measured in serum, i.e., circulating half-life, or in other tissues. Half-life may vary from one immunoglobulin or class of immunoglobulin to another. In general, an increase in antibody half-life results in an increase in mean residence time (MRT) in circulation for the antibody administered.

The increase in half-life allows for the reduction in amount of drug given to a patient as well as reducing the frequency of administration. To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG1, IgG2, IgG3, or IgG4) that is responsible for increasing the in vivo serum half-life of the IgG molecule. Alternatively, antibodies of the disclosure with increased half-lives may be generated by modifying amino acid residues identified as involved in the interaction between the Fc and the FcRn receptor (see, for examples, U.S. Pat. Nos. 6,821,505 and 7,083,784; and WO 09/058,492). In addition, the half-life of antibodies of the disclosure may be increase by conjugation to PEG or Albumin by techniques widely utilized in the art. In some embodiments antibodies comprising Fc variant regions of the disclosure have an increased half-life of about 5%, about 10%, about 15%, about 20%, about 25%, about 30%, about 35%, about 40%, about 45%, about 50%, about 60%, about 65%, about 70%, about 80%, about 85%, about 90%, about 95%, about 100%, about 125%, about 150% or more as compared to an antibody comprising a native Fc region. In some embodiments antibodies comprising Fc variant regions have an increased half-life of about 2 fold, about 3 fold, about 4 fold, about 5 fold, about 10 fold, about 20 fold, about 50 fold or more, or is between 2 fold and 10 fold, or between 5 fold and 25 fold, or between 15 fold and 50 fold, as compared to an antibody comprising a native Fc region.

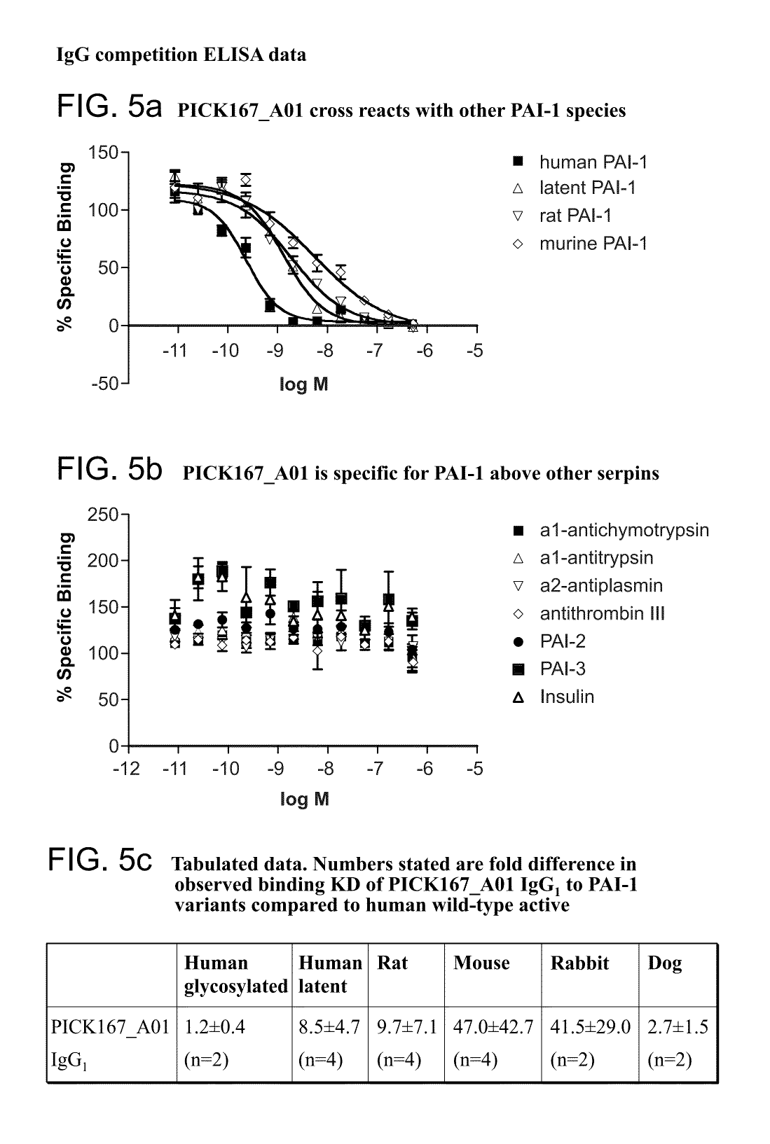

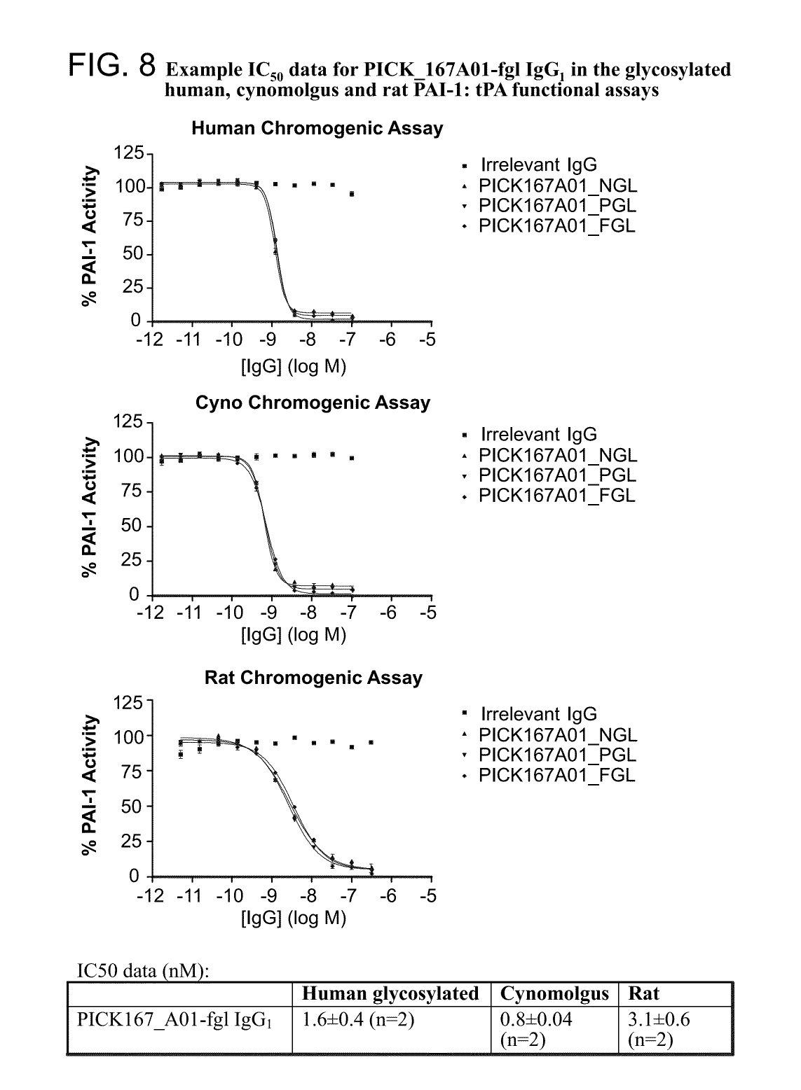

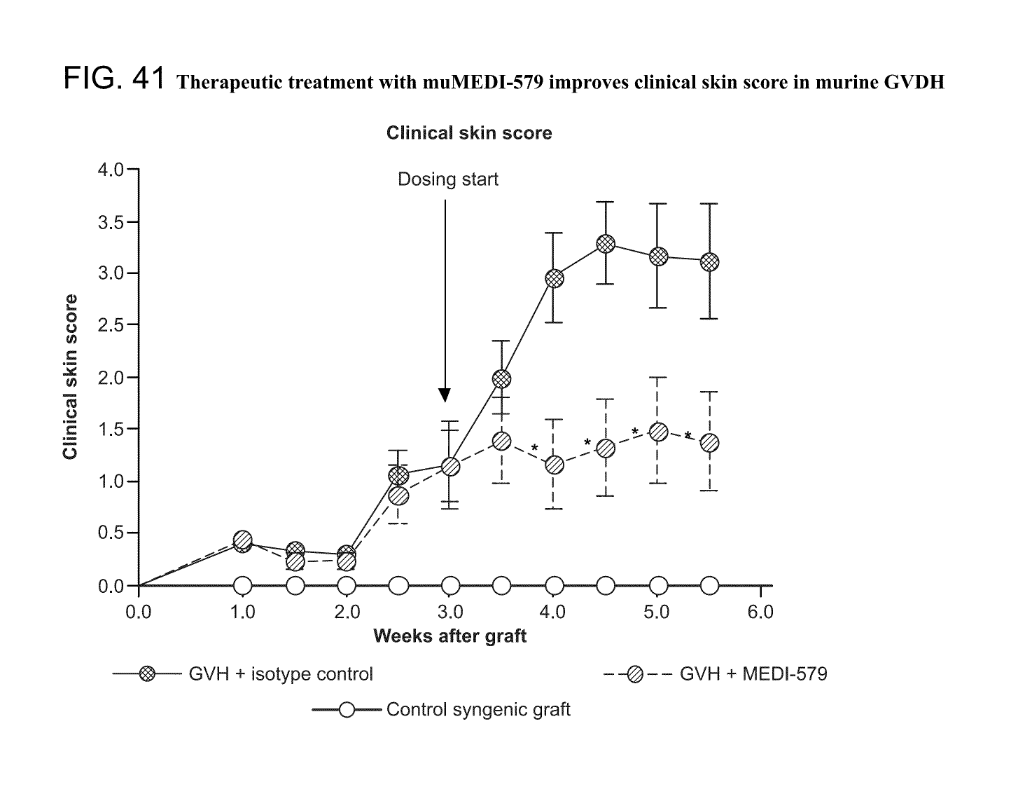

In one embodiment, the present disclosure provides Fc variants, wherein the Fc region comprises a modification (e.g., amino acid substitutions, amino acid insertions, amino acid deletions) at one or more positions chosen from 221, 225, 228, 234, 235, 236, 237, 238, 239, 240, 241, 243, 244, 245, 247, 250, 251, 252, 254, 255, 256, 257, 262, 263, 264, 265, 266, 267, 268, 269, 279, 280, 284, 292, 296, 297, 298, 299, 305, 308, 313, 316, 318, 320, 322, 325, 326, 327, 328, 329, 330, 331, 332, 333, 334, 339, 341, 343, 370, 373, 378, 392, 416, 419, 421, 428, 433, 434, 435, 436, 440, and 443 as numbered by the EU index as set forth in Kabat. Optionally, the Fc region may comprise a modification at additional and/or alternative positions known to one skilled in the art (see, e.g., U.S. Pat. Nos. 5,624,821; 6,277,375; 6,737,056; 7,083,784; 7,317,091; 7,217,797; 7,276,585; 7,355,008; 2002/0,147,311; 2004/0,002,587; 2005/0,215,768; 2007/0,135,620; 2007/0,224,188; 2008/0,089,892; WO 94/29351; and WO 99/58572). Additional, useful amino acid positions and specific substitutions are exemplified in Tables 2, and 6-10 of U.S. Pat. No. 6,737,056; the tables presented in FIG. 41 of US 2006/024298; the tables presented in FIGS. 5, 12, and 15 of US 2006/235208; the tables presented in FIGS. 8, 9 and 10 of US 2006/0173170 and the tables presented in FIGS. 8-10, 13 and 14 of WO 09/058,492.

In a specific embodiment, the present disclosure provides an Fc variant, wherein the Fc region comprises at least one substitution chosen from 221K, 221Y, 225E, 225K, 225W, 228P, 234D, 234E, 234N, 234Q, 234T, 234H, 234Y, 234I, 234V, 234F, 235A, 235D, 235R, 235W, 235P, 235S, 235N, 235Q, 235T, 235H, 235Y, 235I, 235V, 235E, 235F, 236E, 237L, 237M, 237P, 239D, 239E, 239N, 239Q, 239F, 239T, 239H, 239Y, 240I, 240A, 240T, 240M, 241W, 241 L, 241Y, 241E, 241 R. 243W, 243L 243Y, 243R, 243Q, 244H, 245A, 247L, 247V, 247G, 250E, 250Q, 251F, 252L, 252Y, 254S, 254T, 255L, 256E, 256F, 256M, 257C, 257M, 257N, 2621, 262A, 262T, 262E, 263I, 263A, 263T, 263M, 264L, 2641, 264W, 264T, 264R, 264F, 264M, 264Y, 264E, 265A, 265G, 265N, 265Q, 265Y, 265F, 265V, 265I, 265L, 265H, 265T, 266I, 266A, 266T, 266M, 267Q, 267L, 268E, 269H, 269Y, 269F, 269R, 270E, 280A, 284M, 292P, 292L, 296E, 296Q, 296D, 296N, 296S, 296T, 296L, 296I, 296H, 296G, 297S, 297D, 297E, 298A, 298H, 298I, 298T, 298F, 299I, 299L, 299A, 299S, 299V, 299H, 299F, 299E, 305I, 308F313F, 316D, 318A, 318S, 320A, 320S, 322A, 322S, 325Q, 325L, 325I, 325D, 325E, 325A, 325T, 325V, 325H, 326A, 326D, 326E, 326G, 326M, 326V, 327G, 327W, 327N, 327L, 328S, 328M, 328D, 328E, 328N, 328Q, 328F, 328I, 328V, 328T, 328H, 328A, 329F, 329H, 329Q, 330K, 330G, 330T, 330C, 330L, 330Y, 330V, 330I, 330F, 330R, 330H, 331G, 331A, 331L, 331M, 331F, 331W, 331K, 331Q, 331E, 331S, 331V, 331I, 331C, 331Y, 331H, 331R, 331N, 331D, 331T, 332D, 332S, 332W, 332F, 332E, 332N, 332Q, 332T, 332H, 332Y, 332A, 333A, 333D, 333G, 333Q, 333S, 333V, 334A, 334E, 334H, 334L, 334M, 334Q, 334V, 334Y, 339T, 370E, 370N, 378D, 392T, 396L, 416G, 419H, 421K, 428L, 428F, 433K, 433L, 434A, 424F, 434W, 434Y, 436H, 440Y and 443W as numbered by the EU index as set forth in Kabat. Optionally, the Fc region may comprise additional and/or alternative amino acid substitutions known to one skilled in the art including but not limited too those exemplified in Tables 2, and 6-10 of U.S. Pat. No. 6,737,056; the tables presented in FIG. 41 of US 2006/024298; the tables presented in FIGS. 5, 12, and 15 of US 2006/235208; the tables presented in FIGS. 8, 9 and 10 of US 2006/0173170 and the tables presented in FIGS. 8, 9 and 10 of WO 09/058,492.

In a specific embodiment, the present disclosure provides an Fc variant antibody, wherein the Fc region comprises at least one modification (e.g., amino acid substitutions, amino acid insertions, amino acid deletions) at one or more positions chosen from 228, 234, 235 and 331 as numbered by the EU index as set forth in Kabat. In one embodiment, the modification is at least one substitution chosen from 228P, 234F, 235E, 235F, 235Y, and 331S as numbered by the EU index as set forth in Kabat.

In another specific embodiment, the present disclosure provides an Fc variant antibody, wherein the Fc region is an IgG4 Fc region and comprises at least one modification at one or more positions chosen from 228 and 235 as numbered by the EU index as set forth in Kabat. In still another specific embodiment, the Fc region is an IgG4 Fc region and the non-naturally occurring amino acids are chosen from 228P, 235E and 235Y as numbered by the EU index as set forth in Kabat.

In another specific embodiment, the present disclosure provides an Fc variant, wherein the Fc region comprises at least one non-naturally occurring amino acid at one or more positions chosen from 239, 330 and 332 as numbered by the EU index as set forth in Kabat. In one embodiment, the modification is at least one substitution chosen from 239D, 330L, 330Y, and 332E as numbered by the EU index as set forth in Kabat.

In a specific embodiment, the present disclosure provides an Fc variant antibody, wherein the Fc region comprises at least one non-naturally occurring amino acid at one or more positions chosen from 252, 254, and 256 as numbered by the EU index as set forth in Kabat. In one embodiment, the modification is at least one substitution chosen from 252Y, 254T and 256E as numbered by the EU index as set forth in Kabat. See, U.S. Pat. No. 7,083,784, incorporated herein by reference in its entirety.

In certain embodiments the effector functions elicited by IgG antibodies strongly depend on the carbohydrate moiety linked to the Fc region of the protein (Claudia Ferrara et al., 2006, Biotechnology and Bioengineering 93:851-861). Thus, glycosylation of the Fc region can be modified to increase or decrease effector function (see for examples, Umana et al, 1999, Nat. Biotechnol 17:176-180; Davies et al., 2001, Biotechnol Bioeng 74:288-294; Shields et al, 2002, J Biol Chem 277:26733-26740; Shinkawa et al., 2003, J Biol Chem 278:3466-3473; U.S. Pat. Nos. 6,602,684; 6,946,292; 7,064,191; 7,214,775; 7,393,683; 7,425,446; 7,504,256; U.S. Publication. Nos. 2003/0157108; 2003/0003097; 2009/0010921; POTILLEGENT™ technology (Biowa, Inc. Princeton, N.J.); GLYCOMAB™ glycosylation engineering technology (GLYCART biotechnology AG, Zurich, Switzerland)). Accordingly, in one embodiment the Fc regions of anti-PAI-1 antibodies of the disclosure comprise altered glycosylation of amino acid residues. In another embodiment, the altered glycosylation of the amino acid residues results in lowered effector function. In another embodiment, the altered glycosylation of the amino acid residues results in increased effector function. In a specific embodiment, the Fc region has reduced fucosylation. In another embodiment, the Fc region is afucosylated (see for examples, U.S. Patent Application Publication No. 2005/0226867). In one aspect, these antibodies with increased effector function, specifically ADCC, as generated in host cells (e.g., CHO cells, Lemna minor) engineered to produce highly defucosylated antibody with over 100-fold higher ADCC compared to antibody produced by the parental cells (Mori et al., 2004, Biotechnol Bioeng 88:901-908; Cox et al., 2006, Nat. Biotechnol., 24:1591-7).

Addition of sialic acid to the oligosaccharides on IgG molecules can enhance their anti-inflammatory activity and alters their cytotoxicity (Keneko et al., Science, 2006, 313:670-673; Scallon et al., Mol. Immuno. 2007 March; 44(7):1524-34). The studies referenced above demonstrate that IgG molecules with increased sialylation have anti-inflammatory properties whereas IgG molecules with reduced sialylation have increased immunostimulatory properties (e.g., increase ADCC activity). Therefore, an antibody can be modified with an appropriate sialylation profile for a particular therapeutic application (US Publication No. 2009/0004179 and International Publication No. WO 2007/005786).

In one embodiment, the Fc regions of antibodies of the disclosure comprise an altered sialylation profile compared to the native Fc region. In one embodiment, the Fc regions of antibodies of the disclosure comprise an increased sialylation profile compared to the native Fc region. In another embodiment, the Fc regions of antibodies of the disclosure comprise a decreased sialylation profile compared to the native Fc region.

In one embodiment, the Fc variants of the present disclosure may be combined with other known Fc variants such as those disclosed in Ghetie et al., 1997, Nat. Biotech. 15:637-40; Duncan et al, 1988, Nature 332:563-564; Lund et al., 1991, J. Immunol. 147:2657-2662; Lund et al, 1992, Mol Immunol 29:53-59; Alegre et al, 1994, Transplantation 57:1537-1543; Hutchins et al., 1995, Proc Natl. Acad Sci USA 92:11980-11984; Jefferis et al, 1995, Immunol Lett. 44:111-117; Lund et al., 1995, Faseb J 9:115-119; Jefferis et al, 1996, Immunol Lett 54:101-104; Lund et al, 1996, J Immunol 157:4963-4969; Armour et al., 1999, Eur J Immunol 29:2613-2624; Idusogie et al, 2000, J Immunol 164:4178-4184; Reddy et al, 2000, J Immunol 164:1925-1933; Xu et al., 2000, Cell Immunol 200:16-26; Idusogie et al, 2001, J Immunol 166:2571-2575; Shields et al., 2001, J Biol Chem 276:6591-6604; Jefferis et al, 2002, Immunol Lett 82:57-65; Presta et al., 2002, Biochem Soc Trans 30:487-490); U.S. Pat. Nos. 5,624,821; 5,885,573; 5,677,425; 6,165,745; 6,277,375; 5,869,046; 6,121,022; 5,624,821; 5,648,260; 6,528,624; 6,194,551; 6,737,056; 7,122,637; 7,183,387; 7,332,581; 7,335,742; 7,371,826; 6,821,505; 6,180,377; 7,317,091; 7,355,008; 2004/0,002,587; and WO 99/58572. Other modifications and/or substitutions and/or additions and/or deletions of the Fc domain will be readily apparent to one skilled in the art.

### 13. Glycosylation

In addition to the ability of glycosylation to alter the effector function of antibodies, modified glycosylation in the variable region can alter the affinity of the antibody for a target antigen. In one embodiment, the glycosylation pattern in the variable region of the present antibodies is modified. For example, an aglycoslated antibody can be made (i.e., the antibody lacks glycosylation). Glycosylation can be altered to, for example, increase the affinity of the antibody for a target antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861. One or more amino acid substitutions can also be made that result in elimination of a glycosylation site present in the Fc region (e.g., Asparagine 297 of IgG). Furthermore, aglycosylated antibodies may be produced in bacterial cells which lack the necessary glycosylation machinery.

### 14. Antibody Conjugates

In certain embodiments, the antibodies of the disclosure are conjugated or covalently attached to a substance using methods well known in the art. In one embodiment, the attached substance is a therapeutic agent, a detectable label (also referred to herein as a reporter molecule) or a solid support. Suitable substances for attachment to antibodies include, but are not limited to, an amino acid, a peptide, a protein, a polysaccharide, a nucleoside, a nucleotide, an oligonucleotide, a nucleic acid, a hapten, a drug, a hormone, a lipid, a lipid assembly, a synthetic polymer, a polymeric microparticle, a biological cell, a virus, a fluorophore, a chromophore, a dye, a toxin, a hapten, an enzyme, an antibody, an antibody fragment, a radioisotope, solid matrixes, semi-solid matrixes and combinations thereof. Methods for conjugation or covalently attaching another substance to an antibody are well known in the art.

In certain embodiments, the antibodies of the disclosure are conjugated to a solid support. Antibodies may be conjugated to a solid support as part of the screening and/or purification and/or manufacturing process. Alternatively antibodies of the disclosure may be conjugated to a solid support as part of a diagnostic method or composition. A solid support suitable for use in the present disclosure is typically substantially insoluble in liquid phases. A large number of supports are available and are known to one of ordinary skill in the art. Thus, solid supports include solid and semi-solid matrixes, such as aerogels and hydrogels, resins, beads, biochips (including thin film coated biochips), microfluidic chip, a silicon chip, multi-well plates (also referred to as microtitre plates or microplates), membranes, conducting and nonconducting metals, glass (including microscope slides) and magnetic supports. More specific examples of solid supports include silica gels, polymeric membranes, particles, derivatized plastic films, glass beads, cotton, plastic beads, alumina gels, polysaccharides such as Sepharose, poly(acrylate), polystyrene, poly(acrylamide), polyol, agarose, agar, cellulose, dextran, starch, FICOLL, heparin, glycogen, amylopectin, mannan, inulin, nitrocellulose, diazocellulose, polyvinylchloride, polypropylene, polyethylene (including poly(ethylene glycol)), nylon, latex bead, magnetic bead, paramagnetic bead, superparamagnetic bead, starch and the like.

In some embodiments, the solid support may include a reactive functional group, including, but not limited to, hydroxyl, carboxyl, amino, thiol, aldehyde, halogen, nitro, cyano, amido, urea, carbonate, carbamate, isocyanate, sulfone, sulfonate, sulfonamide, sulfoxide, etc., for attaching the antibodies of the disclosure.

A suitable solid phase support can be selected on the basis of desired end use and suitability for various synthetic protocols. For example, where amide bond formation is desirable to attach the antibodies of the disclosure to the solid support, resins generally useful in peptide synthesis may be employed, such as polystyrene (e.g., PAM-resin obtained from Bachem Inc., Peninsula Laboratories, etc.), POLYHIPE™ resin (obtained from Aminotech, Canada), polyamide resin (obtained from Peninsula Laboratories), polystyrene resin grafted with polyethylene glycol (TENTAGEL™, Rapp Polymere, Tubingen, Germany), polydimethyl-acrylamide resin (available from Milligen/Biosearch, California), or PEGA beads (obtained from Polymer Laboratories).

In certain embodiments, the antibodies of the disclosure are conjugated to labels for purposes of diagnostics and other assays wherein the antibody and/or its associated ligand may be detected. A label conjugated to an antibody and used in the present methods and compositions described herein, is any chemical moiety, organic or inorganic, that exhibits an absorption maximum at wavelengths greater than 280 nm, and retains its spectral properties when covalently attached to an antibody. Labels include, without limitation, a chromophore, a fluorophore, a fluorescent protein, a phosphorescent dye, a tandem dye, a particle, a hapten, an enzyme and a radioisotope.

In certain embodiments, the anti-PAI-1 antibodies are conjugated to a fluorophore. As such, fluorophores used to label antibodies of the disclosure include, without limitation; a pyrene (including any of the corresponding derivative compounds disclosed in U.S. Pat. No. 5,132,432), an anthracene, a naphthalene, an acridine, a stilbene, an indole or benzindole, an oxazole or benzoxazole, a thiazole or benzothiazole, a 4-amino-7-nitrobenz-2-oxa-1,3-diazole (NBD), a cyanine (including any corresponding compounds in U.S. Pat. Nos. 6,977,305 and 6,974,873), a carbocyanine (including any corresponding compounds in U.S. Ser. Nos. 09/557,275; U.S. Pat. Nos. 4,981,977; 5,268,486; 5,569,587; 5,569,766; 5,486,616; 5,627,027; 5,808,044; 5,877,310; 6,002,003; 6,004,536; 6,008,373; 6,043,025; 6,127,134; 6,130,094; 6,133,445; and publications WO 02/26891, WO 97/40104, WO 99/51702, WO 01/21624; EP 1 065 250 A1), a carbostyryl, a porphyrin, a salicylate, an anthranilate, an azulene, a perylene, a pyridine, a quinoline, a borapolyazaindacene (including any corresponding compounds disclosed in U.S. Pat. Nos. 4,774,339; 5,187,288; 5,248,782; 5,274,113; and 5,433,896), a xanthene (including any corresponding compounds disclosed in U.S. Pat. Nos. 6,162,931; 6,130,101; 6,229,055; 6,339,392; 5,451,343; 5,227,487; 5,442,045; 5,798,276; 5,846,737; 4,945,171; U.S. Ser. Nos. 09/129,015 and 09/922,333), an oxazine (including any corresponding compounds disclosed in U.S. Pat. No. 4,714,763) or a benzoxazine, a carbazine (including any corresponding compounds disclosed in U.S. Pat. No. 4,810,636), a phenalenone, a coumarin (including an corresponding compounds disclosed in U.S. Pat. Nos. 5,696,157; 5,459,276; 5,501,980 and 5,830,912), a benzofuran (including an corresponding compounds disclosed in U.S. Pat. Nos. 4,603,209 and 4,849,362) and benzphenalenone (including any corresponding compounds disclosed in U.S. Pat. No. 4,812,409) and derivatives thereof. As used herein, oxazines include resorufins (including any corresponding compounds disclosed in U.S. Pat. No. 5,242,805), aminooxazinones, diaminooxazines, and their benzo-substituted analogs.

In a specific embodiment, the fluorophores conjugated to the antibodies described herein include xanthene (rhodol, rhodamine, fluorescein and derivatives thereof) coumarin, cyanine, pyrene, oxazine and borapolyazaindacene. In other embodiments, such fluorophores are sulfonated xanthenes, fluorinated xanthenes, sulfonated coumarins, fluorinated coumarins and sulfonated cyanines. Also included are dyes sold under the tradenames, and generally known as, Alexa Fluor, DyLight, Cy Dyes, BODIPY, Oregon Green, Pacific Blue, IRDyes, FAM, FITC, and ROX.

The choice of the fluorophore attached to the anti-PAI-1 antibody will determine the absorption and fluorescence emission properties of the conjugated antibody. Physical properties of a fluorophore label that can be used for antibody and antibody bound ligands include, but are not limited to, spectral characteristics (absorption, emission and stokes shift), fluorescence intensity, lifetime, polarization and photo-bleaching rate, or combination thereof. All of these physical properties can be used to distinguish one fluorophore from another, and thereby allow for multiplexed analysis. In certain embodiments, the fluorophore has an absorption maximum at wavelengths greater than 480 nm. In other embodiments, the fluorophore absorbs at or near 488 nm to 514 nm (particularly suitable for excitation by the output of the argon-ion laser excitation source) or near 546 nm (particularly suitable for excitation by a mercury arc lamp). In other embodiment a fluorophore can emit in the NIR (near infra red region) for tissue or whole organism applications. Other desirable properties of the fluorescent label may include cell permeability and low toxicity, for example if labeling of the antibody is to be performed in a cell or an organism (e.g., a living animal).

In certain embodiments, an enzyme is a label and is conjugated to an anti-PAI-1 antibody. Enzymes are desirable labels because amplification of the detectable signal can be obtained resulting in increased assay sensitivity. The enzyme itself does not produce a detectable response but functions to break down a substrate when it is contacted by an appropriate substrate such that the converted substrate produces a fluorescent, colorimetric or luminescent signal. Enzymes amplify the detectable signal because one enzyme on a labeling reagent can result in multiple substrates being converted to a detectable signal. The enzyme substrate is selected to yield the preferred measurable product, e.g. colorimetric, fluorescent or chemiluminescence. Such substrates are extensively used in the art and are well known by one skilled in the art.

In one embodiment, colorimetric or fluorogenic substrate and enzyme combination uses oxidoreductases such as horseradish peroxidase and a substrate such as 3,3′-diaminobenzidine (DAB) and 3-amino-9-ethylcarbazole (AEC), which yield a distinguishing color (brown and red, respectively). Other colorimetric oxidoreductase substrates that yield detectable products include, but are not limited to: 2,2-azino-bis(3-ethylbenzothiazoline-6-sulfonic acid) (ABTS), o-phenylenediamine (OPD), 3,3′,5,5′-tetramethylbenzidine (TMB), o-dianisidine, 5-aminosalicylic acid, 4-chloro-1-naphthol. Fluorogenic substrates include, but are not limited to, homovanillic acid or 4-hydroxy-3-methoxyphenylacetic acid, reduced phenoxazines and reduced benzothiazines, including AMPLEX® Red reagent and its variants (U.S. Pat. No. 4,384,042) and reduced dihydroxanthenes, including dihydrofluoresceins (U.S. Pat. No. 6,162,931) and dihydrorhodamines including dihydrorhodamine 123. Peroxidase substrates that are tyramides (U.S. Pat. Nos. 5,196,306; 5,583,001 and 5,731,158) represent a unique class of peroxidase substrates in that they can be intrinsically detectable before action of the enzyme but are “fixed in place” by the action of a peroxidase in the process described as tyramide signal amplification (TSA). These substrates are extensively utilized to label targets in samples that are cells, tissues or arrays for their subsequent detection by microscopy, flow cytometry, optical scanning and fluorometry.

In another embodiment, a colorimetric (and in some cases fluorogenic) substrate and enzyme combination uses a phosphatase enzyme such as an acid phosphatase, an alkaline phosphatase or a recombinant version of such a phosphatase in combination with a colorimetric substrate such as 5-bromo-6-chloro-3-indolyl phosphate (BCIP), 6-chloro-3-indolyl phosphate, 5-bromo-6-chloro-3-indolyl phosphate, p-nitrophenyl phosphate, or o-nitrophenyl phosphate or with a fluorogenic substrate such as 4-methylumbelliferyl phosphate, 6,8-difluoro-7-hydroxy-4-methylcoumarinyl phosphate (DiFMUP, U.S. Pat. No. 5,830,912) fluorescein diphosphate, 3-O-methylfluorescein phosphate, resorufin phosphate, 9H-(1,3-dichloro-9,9-dimethylacridin-2-one-7-yl) phosphate (DDAO phosphate), or ELF 97, ELF 39 or related phosphates (U.S. Pat. Nos. 5,316,906 and 5,443,986).

Glycosidases, in particular beta-galactosidase, beta-glucuronidase and beta-glucosidase, are additional suitable enzymes. Appropriate colorimetric substrates include, but are not limited to, 5-bromo-4-chloro-3-indolyl beta-D-galactopyranoside (X-gal) and similar indolyl galactosides, glucosides, and glucuronides, o-nitrophenyl beta-D-galactopyranoside (ONPG) and p-nitrophenyl beta-D-galactopyranoside. In one embodiment, fluorogenic substrates include resorufin beta-D-galactopyranoside, fluorescein digalactoside (FDG), fluorescein diglucuronide and their structural variants (U.S. Pat. Nos. 5,208,148; 5,242,805; 5,362,628; 5,576,424 and 5,773,236), 4-methylumbelliferyl beta-D-galactopyranoside, carboxyumbelliferyl beta-D-galactopyranoside and fluorinated coumarin beta-D-galactopyranosides (U.S. Pat. No. 5,830,912).

Additional enzymes include, but are not limited to, hydrolases such as cholinesterases and peptidases, oxidases such as glucose oxidase and cytochrome oxidases, and reductases for which suitable substrates are known.

Enzymes and their appropriate substrates that produce chemiluminescence are preferred for some assays. These include, but are not limited to, natural and recombinant forms of luciferases and aequorins. Chemiluminescence-producing substrates for phosphatases, glycosidases and oxidases such as those containing stable dioxetanes, luminol, isoluminol and acridinium esters are additionally useful.

In another embodiment, haptens such as biotin, are also utilized as labels. Biotin is useful because it can function in an enzyme system to further amplify the detectable signal, and it can function as a tag to be used in affinity chromatography for isolation purposes. For detection purposes, an enzyme conjugate that has affinity for biotin is used, such as avidin-HRP. Subsequently a peroxidase substrate is added to produce a detectable signal.

Haptens also include hormones, naturally occurring and synthetic drugs, pollutants, allergens, affector molecules, growth factors, chemokines, cytokines, lymphokines, amino acids, peptides, chemical intermediates, nucleotides and the like.

In certain embodiments, fluorescent proteins may be conjugated to the antibodies as a label. Examples of fluorescent proteins include green fluorescent protein (GFP) and the phycobiliproteins and the derivatives thereof. The fluorescent proteins, especially phycobiliprotein, are particularly useful for creating tandem dye labeled labeling reagents. These tandem dyes comprise a fluorescent protein and a fluorophore for the purposes of obtaining a larger stokes shift wherein the emission spectra is farther shifted from the wavelength of the fluorescent protein's absorption spectra. This is particularly advantageous for detecting a low quantity of a target in a sample wherein the emitted fluorescent light is maximally optimized, in other words little to none of the emitted light is reabsorbed by the fluorescent protein. For this to work, the fluorescent protein and fluorophore function as an energy transfer pair wherein the fluorescent protein emits at the wavelength that the fluorophore absorbs at and the fluorphore then emits at a wavelength farther from the fluorescent proteins than could have been obtained with only the fluorescent protein. A particularly useful combination is the phycobiliproteins disclosed in U.S. Pat. Nos. 4,520,110; 4,859,582; 5,055,556 and the sulforhodamine fluorophores disclosed in U.S. Pat. No. 5,798,276, or the sulfonated cyanine fluorophores disclosed in U.S. Pat. Nos. 6,977,305 and 6,974,873; or the sulfonated xanthene derivatives disclosed in U.S. Pat. No. 6,130,101 and those combinations disclosed in U.S. Pat. No. 4,542,104. Alternatively, the fluorophore functions as the energy donor and the fluorescent protein is the energy acceptor.

In certain embodiments, the label is a radioactive isotope. Examples of suitable radioactive materials include, but are not limited to, iodine (¹²¹I, ¹²³I, ¹²⁵I) carbon (¹⁴C), sulfur (³⁵S), tritium (³H), indium (¹¹¹In, ¹¹²In, ¹¹³mIn, ¹¹⁵mIn), technetium (⁹⁹Tc, ⁹⁹ mTc), thallium (²⁰¹Ti), gallium (⁶⁸Ga, ⁶⁷Ga), palladium (¹⁰³Pd), molybdenum (⁹⁹Mo), xenon (¹³⁵Xe), fluorine (¹⁸F), ¹⁵³SM, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, and ⁹⁷ Ru.

### E. Methods of Use

### 1. Diagnostic Methods of Use

In certain embodiments, anti-PAI-1 antibodies and compositions thereof of the disclosure may be used in vivo and/or in vitro for detecting PAI-1 expression in cells and tissue or for imaging PAI-1 expressing cells and tissues. In certain embodiments, the antibodies are human antibodies and such antibodies are used to image PAI-1 expression in a living human patient. Given that the anti-PAI-1 antibodies and antibody fragments described herein immuno specifically bind to human PAI-1 and inhibit PAI-1 antivity, such as by inhibiting binding of PAI-1 to uPA, but do not inhibit formation or stability of a PAI-1:vitronectin complex, these antibodies can be used to detect or image PAI-1 expression in a living patient.

Moreover, given that exemplary antibodies of the disclosure specifically bind to human PAI-1, as well as PAI-1 from one or more of mouse, rat, cynomolgous, etc., such antibodies are useful for in vitro or in vivo imaging, testing, analysis, etc., of samples in animal models.

By way of example, diagnostic uses can be achieved, for example, by contacting a sample to be tested, optionally along with a control sample, with the antibody under conditions that allow for formation of a complex between the antibody and PAI-1. Complex formation is then detected (e.g., using an ELISA or by imaging to detect a moiety attached to the antibody). When using a control sample along with the test sample, complex is detected in both samples and any statistically significant difference in the formation of complexes between the samples is indicative of the presence of PAI-1 in the test sample.

In one embodiment, the disclosure provides a method of determining the presence of PAI-1 in a sample suspected of containing PAI-1, said method comprising exposing the sample to an anti-PAI-1 antibody of the disclosure, and determining binding of the antibody to PAI-1 in the sample wherein binding of the antibody to PAI-1 in the sample is indicative of the presence of the PAI-1 in the sample. In one embodiment, the sample is a biological sample.

In certain embodiments, the anti-PAI-1 antibodies may be used to detect the overexpression or amplification of PAI-1 using an in vivo or ex vivo diagnostic assay. In one embodiment, the anti-PAI-1 antibody is added to a sample wherein the antibody binds the PAI-1 to be detected and is tagged with a detectable label (e.g. a radioactive isotope or a fluorescent label) and externally scanning the patient for localization of the label.

Alternatively, or additionally, FISH assays such as the INFORM™ (sold by Ventana, Ariz.) or PATHVISION™ (Vysis, Ill.) may be carried out on formalin-fixed, paraffin-embedded tissue to determine the extent (if any) of PAI-1 expression or overexpression in a sample.

In certain embodiments, the anti-PAI-1 antibodies and compositions thereof of the disclosure may be used in vivo and/or in vitro for diagnosing PAI-1 associated diseases. This can be achieved, for example, by contacting a sample to be tested, optionally along with a control sample, with the antibody under conditions that allow for formation of a complex between the antibody and PAI-1. Complex formation is then detected (e.g., using an ELISA). When using a control sample along with the test sample, complex is detected in both samples and any statistically significant difference in the formation of complexes between the samples is indicative of the presence of PAI-1 in the test sample.

In one embodiment, the disclosure provides a method of determining the presence of PAI-1 in a sample suspected of containing PAI-1, said method comprising exposing the sample to an anti-PAI-1 antibody of the disclosure, and determining binding of the antibody to PAI-1 in the sample wherein binding of the antibody to PAI-1 in the sample is indicative of the presence of the PAI-1 in the sample. In one embodiment, the sample is a biological sample. In one embodiment, the biological sample is from a mammal experiencing or suspected of experiencing a PAI-1 associated disease/disorder.

### 2. Therapeutic Methods of Uses

In certain aspects, the anti-PAI-1 antibodies (including antibody fragments) and compositions thereof of the disclosure may be administered for prevention and/or treatment of a subject in need of treatment for one or more diseases or conditions casued or exacerbated, in whole or in part, by fibrosis and/or inflammation. The disclosure encompasses methods of preventing, treating, maintaining, ameliorating, or inhibiting PAI-1 associated or exacerbated aspects of fibrotic and/or inflammatory diseases or conditions in a mammal, comprising administering a therapeutically effective amount of the anti-PAI-1 antibody to the mammal. The antibody therapeutic compositions can be administered short term (acute) or chronic, or intermittently as directed by physician. In certain embodiments, the subject in need of treatment is a subject having one or more of systemic lupus erythromatosus (SLE), scleroderma, pulmonary fibrosis, wherein pulmonary fibrosis is not IPF, diabetic nephropathy, lupus nephritis, graft versus host disease, glomerulonephritis, focal segmental glomerulosclerosis, membranous nephropathy, mesangial proliferative glomerulonephritis, membranoproliferative glomerulonephritis, renal fibrosis, and COPD. In such embodiments, the method comprises a method of treating any of these foregoing diseases or condition in such a subject in need of treatment.

Administration of an anti-PAI-1 antibody having the functional and/or structural properties described herein can be used alone or can be used as part of an overall treatment regimen appropriate for the particular disease or condition being treated. Such an overall therapeutic regimen may combine administration of an anti-PAI-1 antibody with, for example, one or more other medications, surgery, a dietary regimen, an exercise regimen, and the like.

Treating includes, for example, reducuing the severity, duration, and/or frequency of one or more symptoms of the particular disease or condition. In certain embodiments, treating includes decreasing or eliminating or delaying the need for dialysis and/or surgery. In certain embodiments, treating includes preventing, delaying or reversing disease progression, for example, progress of a kidney disorder to renal failure and/or end stage renal disease. Treating also includes or can be evaluated by measuring a reduction in the need for other treatments following anti-PAI-1 therapy. For example, treating may include or be evaluated by measuring a reduction in a patient's need for pain medication, steroids, immunosuppresants, antibiotics, and the like. The effectiveness of treatment, including reduction in frequency, duration, and/or severity of symptoms, may be assessed based on patient self-reporting, morphological criteria, molecular criteria, medical test of function of the affected tissue or organ, etc.

Any of the anti-PAI-1 antibodies and antibody fragments, as well as compositions, having any one or more of the structural and functional features described herein can be used in a method of treating a human patient in need thereof. Thoughout this section, “anti-PAI-1 antibodies” or “antibodies of the disclosure” should be understood to apply to the use of any such anti-PAI-1 antibodies and antibody fragments and/or compositions. In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3, 4) of the following characteristics:

inhibits binding of human PAI-1 to uPA and tPA;

preferentially binds to active human PAI-1 over latent human PAI-1;

can reduce the level of VCAM-1 in dermal tissues;

can reduce the level of TNF-alpha in dermal tissues;

can stimulate plasmin-mediated activation of MMP-1.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least one or more (e.g., at least 1, 2, 3) of the following characteristics:

affinity (K_{D}) between 5 pm to 200 pM for active human PAI-1, as assessed by surface plasmon resonance;

immunospecifically binds to a human PAI-1:vitronectin complex, but does not disrupt the binding of human PAI-1 to vitronectin;

specifically binds to the reactive center loop of PAI-1 that interacts with tPA and uPA.

In certain embodiments, the anti-PAI-1 antibody or antibody fragment is a human or chimeric antibody or antibody fragment that immunospecifically binds to human PAI-1 and inhibits PAI-1 activity, and which antibody or antibody fragment has at least two or more (e.g., at least 2, 3, 4, 5, 6, 7, 8, 9, 10, 11, 12, 13, 14, 15, etc) of the Desired Characteristics.

In certain embodiments, the at least two characteristics includes preferentially binds to active human PAI-1 over latent human PAI-1. In certain embodiments, the at least two characteristics includes the ability to stimulate plasmin-mediated activation of MMP-1.

In certain embodiments, the anti-PAI-1 antibody has, in addition to the functional features or combination of functional features described above, the structural features (e.g., amino acid sequence of one one or more CDRs) of the specific anti-PAI-1 antibodies disclosed herein.

In certain embodiments, the method comprises administering antibodies of the disclosure (e.g., antibodies or fragments) systemically, such as intravenously, intramuscularly, intraperitoneally, or subcutaneously. In certain embodiments, the method comprises administering antibodies of the disclosure locally to, for example, the lung or kidney. Local administration to the lung may be, for example, by inhalation or intranasal delivery. In certain embodiments, antibodies are administered as part of a regimen with dialysis and the antibodies are administered via a dialysis port.

Illustrative examples of fibrotic diseases and conditions that can be treated, along with animal models that can be used to test the efficacy and safety of the treatment, are provided below.

a) Scleroderma

Scleroderma is a chronic autoimmune disease characterized by fibrosis (or hardening), vascular alterations, and autoantibodies. There are two major forms: limited systemic scleroderma and diffuse systemic scleroderma. The cutaneous symptoms of limited systemic scleroderma affect the hands, arms and face. Patients with this form of scleroderma frequently have one or more of the following complications: calcinosis, Raynaud's phenomenon, esophageal dysfunction, sclerodactyl), and telangiectasias.

Diffuse systemic scleroderma is rapidly progressing and affects a large area of the skin and one or more internal organs, frequently the kidneys, esophagus, heart and/or lungs.

Scleroderma affects the small blood vessels known as arterioles, in all organs. First, the endothelial cells of the arteriole die off apoptotically, along with smooth muscle cells. These cells are replaced by collagen and other fibrous material. Inflammatory cells, particularly CD4+ helper T cells, infiltrate the arteriole, and cause further damage.

The skin manifestations of scleroderma can be painful, can impair use of the affected area (e.g., use of the hands, fingers, toes, feet, etc.) and can be disfiguring. Skin ulceration may occur, and such ulcers may be prone to infection or even gangrene. The ulcerated skin may be difficult or slow to heal. Difficulty in healing skin ulcerations may be particularly exacerbated in patients with impaired circulation, such as those with Raynaud's phenomenon. In certain embodiments, the methods of the present disclosure are used to treat scleroderma, for example skin symptoms of scleroderma. In certain embodiments, treating scleroderma comprises treating skin ulceration, such as digital ulcers. Administration of anti-PAI-1 antibodies of the disclosure can be used to reduce the fibrotic and/or inflammatory symptoms of scleroderma in affected tissue and/or organs.

In addition to skin symptoms/manifestations, scleroderma may also affect the heart, kidney, lungs, joints, and digestive tract. In certain embodiments, treating scleroderma includes treating symptoms of the disease in any one or more of these tissues, such as by reducing fibrotic and/or inflammatory symptoms.

Lung problems are amongst the most serious complications of scleroderma and are responsible for much of the mortality associated with the disease. The two predominant lung conditions associated with scleroderma are pulmonary fibrosis and pulmonary hypertension. A patient with lung involvement may have either or both conditions. Lung fibrosis associated with scleroderma is one example of pulmonary fibrosis that can be treated using the anti-PAI-1 antibodies of the disclosure.

Scleroderma involving the lung causes scarring (pulmonary fibrosis). Such pulmonary fibrosis occurs in about 70% of scleroderma patients, although its progression is typically slow and symptoms vary widely across patients in terms of severity. For patients that do have symptoms associated with pulmonary fibrosis, the symptoms include a dry cough, shortness of breadth, and reduced ability to exercise. About 16% of patients with some level of pulmonary fibrosis develop severe pulmonary fibrosis. Patients with severe pulmonary fibrosis experience significant decline in lung function and alveolitis.

In certain embodiments, the methods of the present disclosure are used to treat scleroderma, for example lung fibrosis associated with scleroderma. Administration of anti-PAI-1 antibodies of the disclosure can be used to reduce the fibrotic symptoms of scleroderma in lung. For example, the methods can be used to improve lung function and/or to reduce the risk of death due to scleroderma.

Kidney involvement is also common in scleroderma patients. Renal fibrosis associated with scleroderma is an example of renal fibrosis that can be treated by administration of an anti-PAI-1 antibody of the disclosure.

Signs of kidney complications include increased levels of protein in the urine and mild hypertension. Although kidney involvement is common, the progression is typically slow and the resulting damage does not usually lead to actual kidney failure. Nevertheless, management of renal fibrosis and the resulting kidney damage is useful for (i) preventing or decreasing hypertension and (ii) preventing potentially fatal complications associated with renal failure.

In certain embodiments, the methods of the present disclosure are used to treat scleroderma, for example kidney fibrosis associated with scleroderma. Administration of anti-PAI-1 antibodies of the disclosure can be used to reduce the fibrotic symptoms of scleroderma in kidney. For example, the methods can be used to improve kidney function, to reduce protein in the urine, to reduce hypertension, and/or to reduce the risk of renal crisis that may lead to fatal renal failure.

The methods of the present disclosure can be used in the treatment of scleroderma. Exemplary symptoms that can be treated include, but are not limited to, pain (including joint pain), swelling, skin ulceration, skin irritation, rash, loss of range of motion, and decreased ability to perform daily tasks. Further symptoms that can be treated include lung function (e.g., lung function can be improved) and kidney function. Improvement in any of these symptoms can be measured by, for example, decrease in the number of swollen joints, decrease in the number of painful joints, increased range of motion, increase in healing of ulcerated tissue, decrease in number and/or severity of skin ulcerations, increased ability to perform daily tasks, decreased skin involvement, decreased reliance on pain or other medication, improvement in patient self-assessment of pain or quality of life, increased lung function, decreased hypertension, decreased protein in urine, etc.

In certain embodiments, methods of treating scleroderma include administering an anti-PAI antibody of the disclosure as part of a therapeutic regimen along with one or more other drugs, biologics, or therapeutic interventions appropriate for scleroderma. In certain embodiments, the additional drug, biologic, or therapeutic intervention is appropriate for particular symptoms associated with scleroderma. By way of example, anti-PAI-1 antibodies may be administered as part of a therapeutic regimen along with one or more immunosuppressive agents, such as methotrexate, cyclophosphamide, azathioprine, and mycophenolate mofetil. By way of further example, anti-PAI-1 antibodies may be administered as part of a therapeutic regimen along with one or more agents designed to increase blood flow, such as blood flow to ulcerated digits (e.g., nifedipine, amlodipine, diltiazem, felodipine, or nicardipine). By way of further example, anti-PAI-1 antibodies may be administered as part of a therapeutic regimen along with one or more agents intended to decrease fibrosis of the skin, such as d-penicillamine, colchicine, PUVA, Relaxin, and cyclosporine. By way of further example, anti-PAI-1 antibodies may be administered as part of a therapeutic regimen along with steroids or broncho-dilators.

Moreover, methods of treatment may include a treatment regimen including a dietary regimen, an exercise regimen, stress management, smoking cessation, acupuncture, massage, and/or physical therapy.

The disease symptoms can be replicated in animal models, and such models can be readily used to confirm efficacy of a therapy or combination therapy. Exemplary models include a rodent model in which scleroderma is induced by treatment with cyclosporine A (Damoiseaux et al., Cyclosporine A-Induced Autoimmunity: An Animal Model for Human Scleroderma. J. Experimental Animal Science, 2000, 41(1-2): 22-26) or bleomycin, as well as transgenic mouse models, and graft-versus-host models (Yamamoto, Characteristics of Animal Models for Scleroderma. Current Rheumatology Reviews, 2005, 1: 101-119; Clark, Animal Models in Scleroderma. Current Rheumatology Reports, 2005, 7(2): 150-155). A graft-versus-host model for scleroderma was used to test antibodies of the disclosure, as is summarized in the Examples.

b) COPD

Chronic obstructive pulmonary disease (COPD) is a major cause of disability, and it's the fourth leading cause of death in the United States. More than 12 million people are currently diagnosed with COPD. An additional 12 million likely have the disease but have yet to be diagnosed. COPD blocks airflow and makes it increasingly difficult for a sufferer to breathe. COPD is caused by damage to the airways that eventually interferes with the exchange of oxygen and carbon dioxide in the lungs. COPD includes chronic obstructive bronchitis and emphysema and often both.

COPD patients, whose lungs are already damaged and whose lung function is already compromised, are at increased risk of complications associated with bacterial and viral infections. COPD exacerbation is the term used to describe a worsening of base line COPD symptoms, typically due to infection.

The Global Initiative for Chronic Obstructive Lung Disease (GOLD) has produced a five-stage classification of COPD severity to guide the therapeutic approach, as well as to track changes in patient condition over time or during an exacerbation. In these patients, stage 0 defines the condition characterized by classic clinical symptoms of cough, sputum, and breathlessness without airflow obstruction (e.g., normal spirometry). Stage I defines patients with a forced expiratory volume in one second (FEV1)/forced vital capacity (FVC) of <70%, and an FEV1 of >80% predicted, with or without chronic symptoms that may or may not be aware of disease status. Stage II (FEV1/FVC <70%, FEV1 30-79%) is split into substages IIa (FEV1 50-79%) and IIb (FEV1 30-49%) according to the greater rate of exacerbation experienced by patients in substage IIb, which in turn is inversely related to health status. However, substage IIb is often referred to in the art and herein as stage III. Finally, stage 1V (FEV1/FVC <70% and either FEV1 <30% pred, hypoxaemia, or clinical signs of right heart failure) is expected to be associated with the worst health status. The disclosure contemplates methods of treating patients having a baseline GOLD score of, for example, stage I or higher, stage II or higher, stage III or higher, stage IV. In certain embodiments, the patient has a GOLD score of stage III or stage 1V (or higher) prior to beginning of treatment.

Anti-PAI-1 antibodies of the disclosure can be used to treat COPD, such as to decrease one or more symptoms of COPD. Without being bound by theory, such antibodies can be used to decrease fibrosis and/or inflammation in the lungs of COPD patients, and thus to improve lung function, and slow lung damage. Thus, in certain embodiments, the disclosure provides a method of treating COPD.

In certain embodiments, the compositions and/or methods of the disclosure are administered to improve symptoms of a subject with COPD. Symptoms of COPD and/or lung fibrosis include, but are not limited to, cough with mucus, shortness of breath (dyspnea) that may get worse with mild activity, fatigue, frequent respiratory infections, wheezing, chest tightness, irregular heart beats (arrhythmias), need for breathing machine and oxygen therapy, right-sided heart failure or cor pulmonale (heart swelling and heart failure due to chronic lung disease), pneumonia, pneumothorax, severe weight loss and malnutrition. Symptoms also include decrease in lung function, as evaluated using one or more standard tests of lung function. In certain embodiments, administration of an anti-PAI-1 antibody decreases lung fibrosis.

In certain embodiments, COPD symptoms are monitored before, during or after treatment. Such monitoring may be used to evaluate efficacy of the treatment and/or to determine whether changes in treatment are warranted. In certain embodiments, monitoring occurs over regular intervals during treatment, such as hourly, daily or weekly. In certain embodiments, monitoring occurs over regular intervals after treatment, such as daily, weekly or monthly. Intervals for monitoring may be readily determined by one of skill in the art based on the severity of the condition. In certain embodiments, COPD symptoms are monitored by a pulmonary function tests such as spirometry. In certain embodiments, COPD symptoms are monitored by chest X-ray and/or a computerized tomography (CT) scan. A chest X-ray or CT scan can show emphysema, which is one of the main causes of COPD. In certain embodiments, COPD symptoms are monitored by arterial blood gas analysis. In certain embodiments, COPD symptoms are monitored by sputum examination. In certain embodiments, efficacy of treatment is evaluated using any one or more of the foregoing tests. In certain embodiments, the treatment decreases the severity, duration, or frequency of any one or more symptoms.

The disclosure contemplates methods of treating COPD comprising administering an anti-PAI-1 antibody alone or as part of a therapeutic regimen combined with one or more other drugs, biologics, or other therapeutic modalities. For example, additional treatment modalities that can be used include, but are not limited to, oxygen therapy, steroidal therapy, smoking cessation, bronchial dilators, lung reduction surgery, and the like. Additionally or alternatively, the antibodies may be used alone or used as part of a regimen for managing or preventing infections that may lead to exacerbation. By way of further example, administration of an anti-PAI-1 antibody of the disclosure can be used as part of a therapeutic regimen with one or more of: bronchodilators, such as ipratropium, tiotropium, salmeterol, or formoterol, steroids, anti-viral agents and antibiotics. In certain embodiments, the method comprises using these antibodies in combination with surgery, such as lung volume reduction surgery or lung transplantation. In certain embodiments, the method comprises using these antibodies in combination with therapy such as oxygen therapy, pulmonary rehabilitation or smoking cessation.

The disease symptoms can be replicated in animal models, and such models are readily used to confirm efficacy of a therapy or combination therapy. Experimental studies of COPD may be performed in animals that have been exposed to noxious chemicals, such as cigarette smoke (Shapiro, Animal Models for Chronic Obstructive Pulmonary Disease. Am. J. Respir. Cell Mol. Biol., 2000, 22(1): 4-7; Martorana et al., Models for COPD Involving Cigarette Smoke. Drug Discovery Today: Disease Models, 2007, 3(3): 225-230; Wright and Churg, Animal models of COPD: Barriers, successes, and challenges, Pulmonary Pharmacology & Therapeutics, 2008, 21(5): 696-698).

c) Pulmonary Fibrosis

Pulmonary fibrosis is the formation or development of excess fibrous connective tissue in the lungs, wherein normal lung tissue is replaced with fibrotic tissue. This scarring leads to stiffness of the lungs and impaired lung structure and function. In humans, pulmonary fibrosis is thought to result from repeated injury to the tissue within and between the tiny air sacs (alveoli) in the lungs. In an experimental setting, a variety of animal models have replicated aspects of the human disease. For example, a foreign agent such as bleomycin, fluorescein isothiocyanate, silica, or asbestos may be instilled into the trachea of an animal (Gharaee-Kermani et al., Animal Models of Pulmonary Fibrosis. Methods Mol. Med., 2005, 117:251-259). The methods of the disclosure comprise treating patients having pulmonary fibrosis, wherein pulmonary fibrosis does not include IPF. Anti-PAI-1 antibodies of the disclosure are administered to decrease lung fibrosis and/or to improve lung function. Improvement in symptoms of lung function include improvement of lung function and/or capacity, decreased fatigue, and improvement in oxygen saturation.

In certain embodiments, methods of treating lung fibrosis include administering an anti-PAI antibody of the disclosure as part of a therapeutic regimen along with one or more other drugs, biologics, or therapeutic interventions appropriate for treating pulmonary fibrosis. By way of example, anti-PAI-1 antibodies may be administered as part of a therapeutic regimen along with one or more immunosuppressive agents, steroids, or bronchio-dilators. Moreover, methods of treatment may include a treatment regimen including a dietary regimen, an exercise regimen, stress management, smoking cessation, acupuncture, massage, and/or physical therapy.

d) Systemic Lupus Erythematosus (SLE)

Systemic lupus erythematosus (SLE) is a chronic, inflammatory autoimmune disorder. It may affect the skin, joints, kidneys, and other organs. Almost all people with SLE have joint pain and most develop arthritis. Frequently affected joints are the fingers, hands, wrists, and knees.

General symptoms of SLE include: arthritis; fatigue; general discomfort, uneasiness or ill feeling (malaise); joint pain and swelling; muscle aches; nausea and vomiting; and skin rash. Additionally symptoms may also include: abdominal pain; blood in the urine; fingers that change color upon pressure or in the cold; numbness and tingling; and red spots on skin. In some patients, SLE has lung or kidney involvement. Without being bound by theory, inflammation and/or fibrosis in lung and kidney damages those organs and leads to symptoms associated with lung and/or kidney damage. In some cases, patients with SLE develop a particular kidney condition called lupus nephritis.

In certain embodiments, the disclosure provides methods of treating SLE by administering an effective amount of an anti-PAI-1 antibody of the disclosure. Administering anti-PAI-1 antibodies can be used to decrease one or more symptoms of SLE. In certain embodiments, administering anti-PAI-1 antibodies is used to treat SLE in a patient with lupus nephritis. In such cases, treating SLE comprises treating lupus nephritis, such as by reducing symptoms of lupus nephritis. In certain embodiments, treating comprises treating the skin symptoms of SLE. In certain embodiments, treating comprises reducing one or more symptoms of lupus nephritis. In certain embodiments, treating comprises reducing, delaying or eliminating the need for dialysis. In certain embodiments, treating comprises reducing, delaying, or eliminating the need for kidney transplantation. In certain embodiments, treating comprises delaying or preventing progression of lupus nephritis to renal failure or end stage renal disease.

Lupus nephritis is an inflammation of the kidney, and is a severe complication of systemic lupus erythematosus (SLE). SLE is characterized by spontaneous B and T cell autoreactivity and multiorgan immune injury. In the kidney, lupus nephritis can lead to debilitating loss of function. Patients with lupus nephritis may eventually develop kidney failure and require dialysis or kidney transplantation. Related complications that can also be treated using the methods of the disclosure include interstitial nephritis, and nephrotic syndrome.

The World Health Organization has divided lupus nephritis into six classes based on the biopsy. This classification was defined in 1982 and revised in 1995. Class I is minimal mesangial glomerulonephritis which is histologically normal on light microscopy but with mesangial deposits on electron microscopy. Class II is based on a finding of mesangial proliferative lupus nephritis. Class III is focal proliferative nephritis. Class IV is diffuse proliferative nephritis. Class V is membranous nephritis and is characterized by extreme edema and protein loss. Class VI is glomerulosclerosis. The disclosure contemplates treating patients having lupus nephritis categorized in any of Class I, II, III, IV, V, or VI. In certain embodiments, the patient has lupus nephritis categorized in Class III or higher, in Class IV or higher, or in Class V or higher. In certain embodiments, the patient has lupus nephritis categorized as Class VI.

Symptoms of lupus nephritis include: blood in the urine, foamy appearance to urine, high blood pressure, protein in the urine, fluid retention, and edema. Other symptoms include signs and symptoms of renal fibrosis and/or kidney failure. If left untreated, lupus nephritis may lead to kidney failure, and even end stage renal disease.

Lupus nephritis can be diagnosed and/or monitored using blood or urine tests, as well as by kidney biopsy. Such tests may be used to monitor improvement of symptoms during or following treatment. In certain embodiments, treatment comprises improvement in any one or more of these indica. By way of example, lupus nephritis can be diagnosed and/or assessed by evaluating blood and/or protein content in the urine. Treating may comprise reducing blood and/or protein content in urine, such as to normal or near normal levels. Lupus nephritis can also be diagnosed and/or assessed by evaluating creatinine and/or urea level in blood, and/or by estimates of glomerular filtration rate based on creatinine score.

The disclosure contemplates methods of treating SLE, including treating lupus nephritis, comprising administering an anti-PAI-1 antibody alone or as part of a therapeutic regimen combined with one or more other drugs, biologics, or other therapeutic modalities. The other modalities selected as part of a therapeutic regimen may be selected depending on the severity of the patient's disease, and the particular symptoms being primarily targeted. By way of example, anti-PAI-1 antibodies may, in certain embodiments, be administered as part of a therapeutic regimen with one or more of: analgesics or other pain management medications (e.g., to help alleviate pain associated with arthritic or other symptoms) anti-inflammatory medications, steroids, immunosuppresant agents, and cytotoxic agents. Further examples of agents and treatment modalities that can be used in combination with anti-PAI-1 antibodies include diet, exercise, stress management, acupuncture, and physical therapy. Other specific examples include the use of agents and therapies specific for alleviating symptoms of lupus nephritis, such as blood pressure lowering medications, protein, potassium, and/or sodium reduced diets, cytotoxic agents, and/or immunosuppresants. Methods of treatment include treatment in combination with dialysis, kidney transplant, or one or more other therapies for kidney failure.

The disease symptoms can be replicated in animal models, and such models are readily used to confirm efficacy of a therapy or combination therapy. To this end, many murine models have been developed (Foster, Relevance of Systemic Lupus Erythematosus Nepthritis Animal Models to Human Disease. Semin Nephrol. 1999, 19(1): 12-24). The models make use of host-graft interactions (Bruijn et al., Murine Chronic Graft-Versus-Host Disease as a Model for Lupus Nephritis. Am J Pathol., 1988, 130(3): 639-641), transgenic mice (Takahashi et al., Suppression of Experimental Lupus Nephritis by Aberrant Expression of the Soluble E-Selectin Gene. Pathology International, 2002, 52(3):175-180), and anti-DNA antibodies (Yung and Chan, Anti-DNA Antibodies in the Pathogenesis of Lupus Nephritis—The Emerging Mechanisms. Autoimmunity Reviews, 2008, 7(4): 317-321). Exemplary animal models, including those that are genetically predisposed to developing SLE, are further described and used in the Examples.

e) Diabetic Nephropathy

Diabetic Nephropathy (DN) is an area of substantial unmet medical need. Diabetes is an epidemic world-wide and one-third of diabetes patients go on to develop DN. In human, DN manifests as a clinical syndrome that is composed of albuminuria, progressively declining glomerular filtration rate (GFR) and increased risk for cardiovascular disease. Diabetic albuminuria is associated with the development of characteristic histo-pathologic features, including ticking of the glomerular basement membrane (GBM) and mesangial expansion. As albuminuria progress and renal insufficiency ensues, glomerulosclerosis, arteriolar hyalinosis and tubulointerstitial fibrosis develop.

Diabetic nephropathy is kidney disease or damage that results as a complication of, for example, diabetes. The condition is exacerbated by high blood pressure, high blood sugar levels, and high cholesterol and lipid levels. The exact cause of diabetic nephropathy is unknown. However, without being bound by theory, it is believed that uncontrolled high blood sugar leads to the development of kidney damage, such as fibrosis and scarring of tissue. The present disclosure provides methods for treating diabetic nephropathy by administering an effective amount of an anti-PAI-1 antibody of the disclosure. In certain embodiments, treating comprises reducing one or more symptoms of diabetic nephropathy. In certain embodiments, treating comprises reducing, delaying or eliminating the need for dialysis. In certain embodiments, treating comprises reducing, delaying, or eliminating the need for kidney transplantation. In certain embodiments, treating comprises delaying, preventing or reversing the progression of diabetic nephropathy to renal failure or end stage renal disease.

Early stage diabetic nephropathy has no symptoms. Over time, the kidney's ability to function starts to decline. Symptoms develop late in the disease and may include: fatigue; foamy appearance or excessive frothing of the urine; frequent hiccups; high blood pressure; generalized itching; headache; nausea and vomiting; poor appetite; swelling of the legs; unintentional weight gain (from fluid buildup). Left untreated and/or over time, kidney function may continue to decline as the kidney becomes increasingly damaged. Kidney damage and dysfunction may progress to renal failure, and even end stage renal disease. Dialysis and/or kidney transplant may be needed if the damage progresses to this point.

Diabetic nephropathy can be diagnosed and/or monitored using blood or urine tests, as well as by kidney biopsy. Such tests may be used to monitor improvement of symptoms during or following treatment. In certain embodiments, treatment comprises improvement in any one or more of these indica. By way of example, diabetic nephropathy can be diagnosed and/or assessed by evaluating blood and/or protein content in the urine. Treating may comprise reducing blood and/or protein content in urine, such as to normal or near normal levels. Diabetic nephropathy can also be diagnosed and/or assessed by evaluating creatinine and/or urea level in blood, and/or by estimates of glomerular filtration rate based on creatinine score.

The disclosure contemplates methods of treating diabetic nephropathy, comprising administering an anti-PAI-1 antibody alone or as part of a therapeutic regimen combined with one or more other drugs, biologics, or other therapeutic modalities. By way of example, anti-PAI-1 antibodies can be administered as part of a therapeutic regimen including one or more of, for example, blood pressure medications, such as angiotensin-converting enzyme (ACE) inhibitors or angiotensin receptor blockers (ARBs). In certain embodiments, anti-PAI-1 antibodies are administered as part of a regimen that includes dialysis and/or kidney transplantation.

By way of further example, anti-PAI-1 antibodies can be administered as part of a regimen to control and/or manage diabetes, such as using one or more of diet, exercise, and/or insulin.

The disease symptoms can be replicated in animal models, and such models are readily used to confirm efficacy of a therapy or combination therapy. For example, mouse models have been developed (Breyer et al., Mouse Models of Diabetic Nephropathy. J Am Soc Nephrol, 2005, 16: 27-45), and chemical injections in rats can lead to metabolic abnormalities that resemble diabetic nephropathy in humans (Yokozawa et al., Animal Model of Diabetic Nephropathy. Experimental and Toxicological Pathology, 2001, 53(5): 359-363). Exemplary animal models used to evaluate anti-PAI-1 antibodies of the disclosure are provided in the Examples.

### 3. Kidney Diseases and Disorders

Kidney failure can be caused by a heterogeneous group of disorders. Progressive kidney dysfunction leads to kidney damage, proteinuria, and renal insufficiency. As patient health deteriorates, dialysis may be necessary simply to forestall the damage to the kidney, and to prevent multi-system failure. Patient health and quality of life while on dialysis may be significantly impaired. Over time, kidney failure and renal insufficiency can progress to end stage renal disease requiring transplantation.

In certain embodiments, the disclosure provides a method of treating kidney diseases and disorders caused or exacerbated by fibrosis and/or inflammation. Certain specific conditions, such as diabetic nephropathy and lupus nephritis are described above. Additional description of kidney diseases, generally, as well as other specific conditions are provided here. It is noted that descriptions of symptoms of kidney diseases, methods of evaluating, complications, combination therapies, and the like should be understood to also apply to treatment of diabetic nephropathy and/or lupus nephritis, where applicable.

By way of example, in certain embodiments, anti-PAI-1 antibodies of the disclosure are used to treat or ameliorate (including treating or ameliorating the symptoms of a disease) glomerular diseases such as focal segmental glomerulosclerosis and nephrotic syndrome. Exemplary symptoms that can be treated include, but are not limited to, hypertension, proteinuria, hyperlipidemia, hematuria, and hypercholestermia.

In certain embodiments, the anti-PAI-1 antibodies of the disclosure are administered in combination with one or more agents or treatment modalities appropriate for the underlying kidney disease or condition. In certain embodiments, the anti-PAI-1 antibodies are administered in combination with a dialysis or plasmapheresis regimen. In certain embodiments, the anti-PAI-1 antibodies are used to decrease the frequency with which dialysis or plasmapheresis is required. In certain other embodiments, the anti-PAI-1 antibodies are used in combination with kidney transplantation. In certain other embodiments, the anti-PAI-1 antibodies are used to control renal insufficiency in patients awaiting kidney transplantation.

In certain embodiments, the kidney disorder is focal segmental glomerulosclerosis (FSGS). Glomerular diseases damage the glomeruli, letting protein and sometimes red blood cells leak into the urine. Sometimes a glomerular disease also interferes with the clearance of waste products by the kidney, so they begin to build up in the blood.

Symptoms of glomerular disease include proteinuria, hematuria, reduced glomerular filtration rate, hypoproteinemia, and edema. A number of different diseases can result in glomerular disease. It may be the direct result of an infection or a drug toxic to the kidneys, or it may result from a disease that affects the entire body, like diabetes or lupus. FSGS is one particular glomerular disease, but even this particular condition characterized by scarring in the kidney can have numerous causes. Patients with FSGS typically progress to end stage renal disease within 5-20 years, although patients with aggressive forms of the disease progress to ESRD in 2 to 3 years.

In certain embodiments, anti-PAI-1 antibodies are used alone or in combination with, for example, steroids, immunosuppressive drugs, and/or ACE inhibitors to treat FSGS.

The primary pathophysiologic process in FSGS is an injury to podocytes, with proliferation of mesangial, endothelial, and epithelial cells in the early stages followed by shrinkage and/or collapse of glomerular capillaries and, ultimately, sclerosis. FSGS initially begins in the deeper juxtamedullary glomeruli and subsequently extends to the superficial nephrons. The characteristic lesion is a segmental solidification of the glomerular tuft, usually in the perihilar region and sometimes in the peripheral areas, including the tubular pole. The extent of lesions varies in different portions of the kidney, ranging from normal unaffected glomerulus to segmental sclerosis and, eventually, global glomerulosclerosis as the disease evolves. Diffuse foot process fusion occurs, predominantly in the sclerotic segments, although partial effacement may be observed in normal-appearing lobules.

FSGS can be classified as primary (idiopathic) or secondary. Primary FSGS includes the following: FSGS with hyalinosis, progression from minimal-change disease, progression from immunoglobulin M (IgM) nephropathy, progression from mesangial proliferative glomerulonephritis. Secondary FSGS may result from drug use (e.g., intravenous heroin), infection with certain viruses (e.g., hepatitis B, HIV, parvovirus), hemodynamic factors with reduced renal mass (e.g., solitary kidney, renal allograft, renal dysplasia, renal agenesis, oligomeganephronia, segmental hypoplasia, vesicoureteric reflux), hemodynamic causes without reduced renal mass (e.g., massive obesity, sickle cell nephropathy, congenital cyanotic heart disease), certain malignancies (e.g., lymphomas), hypertensive nephrosclerosis, Alport syndrome, sarcoidosis, and radiation nephritis.

Typically, idiopathic FSGS is observed in persons aged 18-45 years, although no age group is exempt from the disease. In adults, the lesion is more common in men and is observed in 20-30% of patients with nephrotic syndrome.

Other types of glomerular diseases include minimal change disease. Minimal Change Disease (MCD) is the diagnosis given when a patient has nephrotic syndrome and the kidney biopsy reveals little or no change to the structure of glomeruli or surrounding tissues when examined by a light microscope. Thus, in contrast to FSGS which is characterized by scarring in the kidney, patients with MCD have symptoms of renal insufficiency without kidney scarring. Tiny drops of a fatty substance called a lipid may be present, but no scarring has taken place within the kidney. MCD may occur at any age, but it is most common in childhood.

Currently, patients with MCD are treated with steroid therapy. However, a small percentage of patients with idiopathic nephrotic syndrome do not respond to steroid therapy. For these patients, the doctor may recommend a low-sodium diet and prescribe a diuretic to control edema, and may also employ immunosuppressant drugs, such as chlorambucil, cyclophosphamide, or cyclosporine. Given the side effects of long term treatment with steroid and/or immunosuppressant drugs, there exists a need for improved methods and compositions for treating or ameliorating the symptoms of MCD.

Left untreated, kidney diseases, regardless of cause, can lead to renal failure. Renal failure is any acute or chronic loss of kidney function and is the term used when some kidney function remains. End stage renal disease (ESRD) is total, or nearly total, permanent kidney failure. Depending on the form of glomerular disease, renal function may be lost in a matter of days or weeks or may deteriorate slowly and gradually over the course of decades. Once a patient has progressed to ESRD, dialysis (hemidialysis or peritoneal dialysis) is required to prevent death. Patients must remain on some form of dialysis regimen or must obtain a kidney transplant.

Nephrotic syndrome is a disorder where the kidneys have been damaged, causing them to leak protein from the blood into the urine. It is a fairly benign disease when it occurs in childhood, but may lead to chronic renal failure, especially in adults, or be a sign of an underlying serious disease such as systemic lupus erythematosus or a malignancy. Nephrotic syndrome can be caused by any of a number of conditions including FSGS.

The most common sign of nephrotic syndrome is excess fluid in the body. This may take several forms including puffiness around the eyes, edema over the legs with pitting, fluid in the pleural cavity causing pleural effusion, fluid in the peritoneal cavity causing ascites, renal failure, hypertension, and proteinuria.

Renal insufficiency leading, eventually, to ESRD is a significant public health problem. Even with improved methods of dialysis and transplantation, the mortality associated with ESRD is extremely high. Accordingly, there is a significant need for improved methods and compositions for treating the symptoms of renal insufficiency, regardless of its cause, and for helping to prevent or slow the progression to ESRD. Anti-PAI-1 antibodies can be used in this way.

Renal insufficiency, also called renal failure, is when the kidneys no longer have enough kidney function to maintain a normal state of health. The incidence of renal failure is approximately 200 cases per 1 million persons in the United States. Causes include diseases of the kidney such as FSGS and nephrotic syndrome, as well as other diseases and conditions that have a secondary impact on kidney function including type I and type II diabetes, hypertension, urologic diseases, vasculitis, systemic lupus erythematosus, and drug toxicity. As detailed above, over time renal insufficiency can lead to ESRD.

The present disclosure provides anti-PAI-1 antibodies for use in the treatment of kidney diseases and conditions. For example, administration of anti-PAI-1 antibodies can be used to treat or ameliorate one or more symptoms of any of the disease or conditions. In certain embodiments, treating comprises improving renal function, decreasing proteinuria, improving hypertension, and/or decreasing renal fibrosis. In certain embodiments, treating comprises delaying or preventing progression to renal insufficiency, renal failure, or ESRD; (ii) delaying, reducing, or preventing need for dialysis; (iii) delaying or preventing need for kidney transplantation.

Anti-PAI-1 antibodies of the disclosure can be used alone, or as part of a therapeutic regimen appropriate for the particular underlying kidney disease or disorder. By way of example, a therapeutic regimen may include one or more of: diurectics; ACE inhibitors; angiotensin receptor antagonists; drugs that target the renin pathway; dialysis; a dietary regimen; or transplantation.

The disclosure contemplates methods of treating any of the foregoing diseases and conditions. By treating is meant to include decreasing or ameliorating the disease, including one or more symptoms of the disease or condition. The method comprises administering to a subject in need thereof an effective amount of an anti-PAI-1 antibody of the disclosure. In certain embodiments, antibodies of the disclosure are administered as part of a therapeutic regimen with (at the same or a different time) one or more other drugs, biologics, or other therapeutic modalities appropriate for the disease or condition. In certain embodiments, antibodies of the disclosure are administered in combination. Antibodies of the disclosure are administered via a route appropriate for the disease or condition, including systemically or locally. In certain embodiments, antibodies of the disclosure are administered in combination with dialysis, and the antibody is administered via a dialysis port.

It should be appreciated that, throughout this section, references to anti-PAI-1 antibodies and anti-PAI-1 antibodies of the disclosure should be understood to include antibodies and antibody fragments. Further, it should be understood that any of the anti-PAI-1 antibodies described herein, including pharmaceutical compositions comprising such antibodies, can be used in methods of treating any of the diseases or conditions.

### F. Pharmaceutical Formulations

This section of the specification describes various exemplary preparations and formulations comprising anti-PAI-1 antibodies (including antibody fragments) of the present disclosure. It should be understood that any of the anti-PAI-1 antibodies and antibody fragments described herein, including antibodies and antibody fragments having any one or more of the structural and functional features described in detail throughout the application, may be formulated or prepared as described below. When various formulations are described in this section as including an antibody, it is understood that such an antibody may be an antibody or an antibody fragment having any one or more of the characteristics of the anti-PAI-1 antibodies and antibody fragments described herein. Any such antibodies and formulations may be used and administered as part of the methods of treatment described herein. The disclosure contemplates all combinations of any of the aspects and embodiments of the disclosure.

In certain embodiments, the anti-PAI-1 antibodies of the disclosure may be formulated with a pharmaceutically acceptable carrier as pharmaceutical (therapeutic) compositions, and may be administered by a variety of methods known in the art. As will be appreciated by the skilled artisan, the route and/or mode of administration will vary depending upon the desired results. As used herein, the pharmaceutical formulations comprising the anti-PAI-1 antibodies are referred to as formulations of the disclosure. The term “pharmaceutically acceptable carrier” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also routinely contain compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the antibodies of the present disclosure, and with each other, in a manner such that there is no interaction which would substantially impair the desired pharmaceutical efficacy.

The formulations of the disclosure are present in a form known in the art and acceptable for theurapeutic uses. In one embodiment, a formulation of the disclosure is a liquid formulation. In another embodiment, a formulation of the disclosure is a lyophilized formulation. In a further embodiment, a formulation of the disclosure is a reconstituted liquid formulation. In one embodiment, a formulation of the disclosure is a stable liquid formulation. In one embodiment, a liquid formulation of the disclosure is an aqueous formulation. In another embodiment, the liquid formulation is non-aqueous. In a specific embodiment, a liquid formulation of the disclosure is an aqueous formulation wherein the aqueous carrier is distilled water.

The formulations of the disclosure comprise an anti-PAI-1 antibody in a concentration resulting in a w/v appropriate for a desired dose. In certain embodiments, the anti-PAI-1 antibody is present in the formulation of the disclosure at a concentration of about 1 mg/ml to about 500 mg/ml. In one embodiment, the concentration of anti-PAI-1 antibody, which is included in the formulation of the disclosure, is at least 1 mg/ml, at least 5 mg/ml, at least 10 mg/ml, at least 15 mg/ml, at least 20 mg/ml, at least 25 mg/ml, at least 30 mg/ml, at least 35 mg/ml, at least 40 mg/ml, at least 45 mg/ml, at least 50 mg/ml, at least 55 mg/ml, at least 60 mg/ml, at least 65 mg/ml, at least 70 mg/ml, at least 75 mg/ml, at least 80 mg/ml, at least 85 mg/ml, at least 90 mg/ml, at least 95 mg/ml, at least 100 mg/ml, at least 105 mg/ml, at least 110 mg/ml, at least 115 mg/ml, at least 120 mg/ml, at least 125 mg/ml, at least 130 mg/ml, at least 135 mg/ml, at least 140 mg/ml, at least 150 mg/ml, at least 200 mg/ml, at least 250 mg/ml, or at least 300 mg/ml.

In a specific embodiment, a formulation of the disclosure comprises at least about 100 mg/ml of an anti-PAI-1 antibody of the disclosure. In a specific embodiment, a formulation of the disclosure comprises at least about 125 mg/ml of an anti-PAI-1 antibody of the disclosure. In a specific embodiment, a formulation of the disclosure comprises at least about 130 mg/ml of an anti-PAI-1 antibody of the disclosure. In a specific embodiment, a formulation of the disclosure comprises at least about 150 mg/ml of an anti-PAI-1 antibody of the disclosure.

In one embodiment, the concentration of anti-PAI-1 antibody, which is included in the formulation of the disclosure, is between about 1 mg/ml and about 25 mg/ml, between about 1 mg/ml and about 200 mg/ml, between about 25 mg/ml and about 200 mg/ml, between about 50 mg/ml and about 200 mg/ml, between about 75 mg/ml and about 200 mg/ml, between about 100 mg/ml and about 200 mg/ml, between about 125 mg/ml and about 200 mg/ml, between about 150 mg/ml and about 200 mg/ml, between about 25 mg/ml and about 150 mg/ml, between about 50 mg/ml and about 150 mg/ml, between about 75 mg/ml and about 150 mg/ml, between about 100 mg/ml and about 150 mg/ml, between about 125 mg/ml and about 150 mg/ml, between about 25 mg/ml and about 125 mg/ml, between about 50 mg/ml and about 125 mg/ml, between about 75 mg/ml and about 125 mg/ml, between about 100 mg/ml and about 125 mg/ml, between about 25 mg/ml and about 100 mg/ml, between about 50 mg/ml and about 100 mg/ml, between about 75 mg/ml and about 100 mg/ml, between about 25 mg/ml and about 75 mg/ml, between about 50 mg/ml and about 75 mg/ml, or between about 25 mg/ml and about 50 mg/ml.

In a specific embodiment, a formulation of the disclosure comprises between about 90 mg/ml and about 110 mg/ml of an anti-PAI-1 antibody of the disclosure. In another specific embodiment, a formulation of the disclosure comprises between about 100 mg/ml and about 210 mg/ml of an anti-PAI-1 antibody of the disclosure.

In other certain embodiments, the formulations of the disclosure comprising an anti-PAI-1 antibody may further comprise one or more active compounds as necessary for the particular indication being treated, preferably those with complementary activities that do not adversely affect each other. Such additional active compound/s is/are suitably present in combination in amounts that are effective for the purpose intended.

According to certain aspects of the disclosure, the formulations of the disclosure may be prepared for storage by mixing the anti-PAI-1 antibody having the desired degree of purity with optional physiologically acceptable carriers, excipients or stabilizers, including, but not limited to buffering agents, saccharides, salts, surfactants, solubilizers, polyols, diluents, binders, stabilizers, salts, lipophilic solvents, amino acids, chelators, preservatives, or the like (Remington's Pharmaceutical Sciences, 16th edition, Osol, A. Ed. (1999)), in the form of lyophilized formulations or aqueous solutions at a desired final concentration. Acceptable carriers, excipients, or stabilizers are nontoxic to recipients at the dosages and concentrations employed, and include buffers such as histidine, phosphate, citrate, glycine, acetate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrolidone; amino acids such as glycine, glutamine, asparagine, histidine, arginine, or lysine; monosaccharides, disaccharides, and other carbohydrates including trehalose, glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN, polysorbate 80, PLURONICS™ or polyethylene glycol (PEG).

In one aspect, the buffering agent is chosen from histidine, citrate, phosphate, glycine, and acetate. In another aspect the saccharide excipient is chosen from trehalose, sucrose, mannitol, maltose and raffinose. In still other aspects the surfactant is chosen from polysorbate 20, polysorbate 40, polysorbate 80, and Pluronic F68. In yet other embodiments the salt is chosen from NaCl, KCl, MgCl₂, and CaCl₂

The formulations of the disclosure may include a buffering or pH adjusting agent to provide improved pH control. In one embodiment, a formulation of the disclosure has a pH of between about 3.0 and about 9.0, between about 4.0 and about 8.0, between about 5.0 and about 8.0, between about 5.0 and about 7.0, between about 5.0 and about 6.5, between about 5.5 and about 8.0, between about 5.5 and about 7.0, or between about 5.5 and about 6.5. In a further embodiment, a formulation of the disclosure has a pH of about 3.0, about 3.5, about 4.0, about 4.5, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.5, about 8.0, about 8.5, or about 9.0. In a specific embodiment, a formulation of the disclosure has a pH of about 6.0. One of skill in the art understands that the pH of a formulation generally should not be equal to the isoelectric point of the particular anti-PAI-1 antibody to be used in the formulation.

Typically, the buffering agent is a salt prepared from an organic or inorganic acid or base. Representative buffering agents include, but are not limited to, organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. In addition, amino acid components can also function in a buffering capacity. Representative amino acid components which may be utilized in the formulations of the disclosure as buffering agents include, but are not limited to, glycine and histidine. In certain embodiments, the buffering agent is chosen from histidine, citrate, phosphate, glycine, and acetate. In a specific embodiment, the buffering agent is histidine. In another specific embodiment, the buffering agent is citrate. In yet another specific embodiment, the buffering agent is glycine. The purity of the buffering agent should be at least 98%, or at least 99%, or at least 99.5%. As used herein, the term “purity” in the context of histidine and glycine refers to chemical purity of histidine or glycine as understood in the art, e.g., as described in The Merck Index, 13th ed., O'Neil et al. ed. (Merck & Co., 2001).

Buffering agents are typically used at concentrations between about 1 mM and about 200 mM or any range or value therein, depending on the desired ionic strength and the buffering capacity required. The usual concentrations of conventional buffering agents employed in parenteral formulations can be found in: Pharmaceutical Dosage Form: Parenteral Medications, Volume 1, 2^{nd} Edition, Chapter 5, p. 194, De Luca and Boylan, “Formulation of Small Volume Parenterals”, Table 5: Commonly used additives in Parenteral Products. In one embodiment, the buffering agent is at a concentration of about 1 mM, or of about 5 mM, or of about 10 mM, or of about 15 mM, or of about 20 mM, or of about 25 mM, or of about 30 mM, or of about 35 mM, or of about 40 mM, or of about 45 mM, or of about 50 mM, or of about 60 mM, or of about 70 mM, or of about 80 mM, or of about 90 mM, or of about 100 mM. In one embodiment, the buffering agent is at a concentration of 1 mM, or of 5 mM, or of 10 mM, or of 15 mM, or of 20 mM, or of 25 mM, or of 30 mM, or of 35 mM, or of 40 mM, or of 45 mM, or of 50 mM, or of 60 mM, or of 70 mM, or of 80 mM, or of 90 mM, or of 100 mM. In a specific embodiment, the buffering agent is at a concentration of between about 5 mM and about 50 mM. In another specific embodiment, the buffering agent is at a concentration of between 5 mM and 20 mM.

In certain embodiments, the formulation of the disclosure comprises histidine as a buffering agent. In one embodiment the histadine is present in the formulation of the disclosure at a concentration of at least about 1 mM, at least about 5 mM, at least about 10 mM, at least about 20 mM, at least about 30 mM, at least about 40 mM, at least about 50 mM, at least about 75 mM, at least about 100 mM, at least about 150 mM, or at least about 200 mM histidine. In another embodiment, a formulation of the disclosure comprises between about 1 mM and about 200 mM, between about 1 mM and about 150 mM, between about 1 mM and about 100 mM, between about 1 mM and about 75 mM, between about 10 mM and about 200 mM, between about 10 mM and about 150 mM, between about 10 mM and about 100 mM, between about 10 mM and about 75 mM, between about 10 mM and about 50 mM, between about 10 mM and about 40 mM, between about 10 mM and about 30 mM, between about 20 mM and about 75 mM, between about 20 mM and about 50 mM, between about 20 mM and about 40 mM, or between about 20 mM and about 30 mM histidine. In a further embodiment of the disclosure comprises about 1 mM, about 5 mM, about 10 mM, about 20 mM, about 25 mM, about 30 mM, about 35 mM, about 40 mM, about 45 mM, about 50 mM, about 60 mM, about 70 mM, about 80 mM, about 90 mM, about 100 mM, about 150 mM, or about 200 mM histidine. In a specific embodiment, a formulation of the disclosure comprises about 10 mM histidine. In another specific embodiment, a formulation of the disclosure comprises about 25 mM histidine. In yet another specific embodiment, a formulation of the disclosure comprises about 0 mM histidine.

In certain embodiments, the formulations of the disclosure may comprise a carbohydrate excipient. Carbohydrate excipients can act, e.g., as viscosity enhancing agents, stabilizers, bulking agents, solubilizing agents, and/or the like. Carbohydrate excipients are generally present at between about 1% to about 99% by weight or volume. In one embodiment, the carbohydrate excipient is present at between about 0.1% to about 20%. In another embodiment, the carbohydrate excipient is present at between about 0.1% to about 15%. In a specific embodiment, the carbohydrate excipient is present at between about 0.1% to about 5%, or between about 1% to about 20%, or between about 5% to about 15%, or between about 8% to about 10%, or between about 10% and about 15%, or between about 15% and about 20%. In another specific embodiment, the carbohydrate excipient is present at between 0.1% to 20%, or between 5% to 15%, or between 8% to 10%, or between 10% and 15%, or between 15% and 20%. In still another specific embodiment, the carbohydrate excipient is present at between about 0.1% to about 5%. In still another specific embodiment, the carbohydrate excipient is present at between about 5% to about 10%. In yet another specific embodiment, the carbohydrate excipient is present at between about 15% to about 20%. In still other specific embodiments, the carbohydrate excipient is present at 1%, or at 1.5%, or at 2%, or at 2.5%, or at 3%, or at 4%, or at 5%, or at 10%, or at 15%, or at 20%.

Carbohydrate excipients suitable for use in the formulations of the disclosure include, but are not limited to, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and the like. In one embodiment, the carbohydrate excipients for use in the present disclosure are chosen from, sucrose, trehalose, lactose, mannitol, and raffinose. In a specific embodiment, the carbohydrate excipient is trehalose. In another specific embodiment, the carbohydrate excipient is mannitol. In yet another specific embodiment, the carbohydrate excipient is sucrose. In still another specific embodiment, the carbohydrate excipient is raffinose. The purity of the carbohydrate excipient should be at least 98%, or at least 99%, or at least 99.5%.

In a specific embodiment, the formaultions of the disclosure comprise trehalose. In one embodiment, a formulation of the disclosure comprises at least about 1%, at least about 2%, at least about 4%, at least about 8%, at least about 20%, at least about 30%, or at least about 40% trehalose. In another embodiment, a formulation of the disclosure comprises between about 1% and about 40%, between about 1% and about 30%, between about 1% and about 20%, between about 2% and about 40%, between about 2% and about 30%, between about 2% and about 20%, between about 4% and about 40%, between about 4% and about 30%, or between about 4% and about 20% trehalose. In a further embodiment, a formulation of the disclosure comprises about 1%, about 2%, about 4%, about 6%, about 8%, about 15%, about 20%, about 30%, or about 40% trehalose. In a specific embodiment, a formulation of the disclosure comprises about 4%, about 6% or about 15% trehalose.

In certain embodiments, a formulation of the disclosure comprises an excipient. In a specific embodiment, a formulation of the disclosure comprises at least one excipient chosen from: sugar, salt, surfactant, amino acid, polyol, chelating agent, emulsifier and preservative. In one embodiment, a formulation of the disclosure comprises a salt. In one embodiment, a formulation of the disclosure comprises a salt chosen from: NaCl, KCl, CaCl₂, and MgCl₂. In a specific embodiment, a formulation of the disclosure comprises NaCl.

In a specific embodiment, a formulation of the disclosure comprises at least about 10 mM, at least about 25 mM, at least about 50 mM, at least about 75 mM, at least about 80 mM, at least about 100 mM, at least about 125 mM, at least about 150 mM, at least about 175 mM, at least about 200 mM, or at least about 300 mM sodium chloride (NaCl). In a further embodiment, a formulation described herein comprises between about 10 mM and about 300 mM, between about 10 mM and about 200 mM, between about 10 mM and about 175 mM, between about 10 mM and about 150 mM, between about 25 mM and about 300 mM, between about 25 mM and about 200 mM, between about 25 mM and about 175 mM, between about 25 mM and about 150 mM, between about 50 mM and about 300 mM, between about 50 mM and about 200 mM, between about 50 mM and about 175 mM, between about 50 mM and about 150 mM, between about 75 mM and about 300 mM, between about 75 mM and about 200 mM, between about 75 mM and about 175 mM, between about 75 mM and about 150 mM, between about 100 mM and about 300 mM, between about 100 mM and about 200 mM, between about 100 mM and about 175 mM, or between about 100 mM and about 150 mM sodium chloride. In a further embodiment, a formulation of the disclosure comprises about 10 mM. about 25 mM, about 50 mM, about 75 mM, about 80 mM, about 100 mM, about 125 mM, about 150 mM, about 175 mM, about 200 mM, or about 300 mM sodium chloride.

In certain embodiments, a formulation of the disclosure comprises an amino acid. In one embodiment, a formulation of the disclosure comprises an amino acid salt. In one embodiment, a formulation of the disclosure comprises an amino acid chosen from lysine, arginine, glycine and histidine. In one embodiment, a formulation of the disclosure comprises at least about 1 mM of an amino acid, at least about 10 mM of an amino acid, at least about 25 mM of an amino acid, at least about 50 mM of an amino acid, at least about 100 mM of an amino acid, at least about 150 mM of an amino acid, at least about 200 mM of an amino acid, at least about 250 mM of an amino acid, at least about 300 mM of an amino acid, at least about 350 mM of an amino acid, or at least about 400 mM of an amino acid. In another embodiment, a formulation of the disclosure comprises between about 1 mM and about 100 mM, between about 10 mM and about 150 mM, between about 25 mM and about 250 mM, between about 25 mM and about 300 mM, between about 25 mM and about 350 mM, between about 25 mM and about 400 mM, between about 50 mM and about 250 mM, between about 50 mM and about 300 mM, between about 50 mM and about 350 mM, between about 50 mM and about 400 mM, between about 100 mM and about 250 mM, between about 100 mM and about 300 mM, between about 100 mM and about 400 mM, between about 150 mM and about 250 mM, between about 150 mM and about 300 mM, or between about 150 mM and about 400 mM of an amino acid. In a further embodiment, a formulation of the disclosure comprises about 1 mM, 1.6 mM, 25 mM, about 50 mM, about 100 mM, about 150 mM, about 200 mM, about 250 mM, about 300 mM, about 350 mM, or about 400 mM of an amino acid.

In a specific embodiment, a formulation of the disclosure comprises about 200 mM of arginine. In a specific embodiment, a formulation of the disclosure comprises about 1.6 mM of glycine. In a specific embodiment, a formulation of the disclosure comprises about 10 mM of histidine. In a specific embodiment, a formulation of the disclosure comprises about 25 mM of histidine. The formulations of the disclosure may comprise an amino acid alone or incombination with another amino acid.

In one embodiment, a formulation of the disclosure comprises trehalose and an amino acid. In one embodiment, a formulation of the disclosure comprises trehalose and an amino acid at a molar ratio of about 0.1, about 0.5, about 0.75, about 1, about 5, about 10, about 20, about 30, about 40, about 50, about 60, about 70, about 80, about 90, about 100, about 200, or about 300. In one embodiment, a formulation of the disclosure comprises trehalose and an amino acid at a molar ratio of about 1.5, about 1.7, about 1.8, about 1.9, about 2, about 2.1, about 2.2, about 2.3, about 2.4, about 2.5, about 2.6, about 2.7, about 2.8, about 2.9, about 3, about 3.1, about 3.2, about 3.3, about 3.4, about 3.5, or about 4. In a specific embodiment, a formulation of the disclosure comprises trehalose and an amino acid at a molar ratio of about 2.1. In a specific embodiment, a formulation of the disclosure comprises trehalose and an amino acid at a molar ratio of about 2.2. In a specific embodiment, a formulation of the disclosure comprises trehalose and an amino acid at a molar ratio of about 2.4. In a specific embodiment, a formulation of the disclosure comprises trehalose and an amino acid at a molar ratio of about 2.5. In a specific embodiment, a formulation of the disclosure comprises trehalose and an amino acid at a molar ratio of about 2.6. In a specific embodiment, a formulation of the disclosure comprises trehalose and an amino acid at a molar ratio of about 2.7.

The formulations of the disclosure may further comprise a surfactant. The term “surfactant” as used herein refers to organic substances having amphipathic structures; namely, they are composed of groups of opposing solubility tendencies, typically an oil-soluble hydrocarbon chain and a water-soluble ionic group. Surfactants can be classified, depending on the charge of the surface-active moiety, into anionic, cationic, and nonionic surfactants. Surfactants are often used as wetting, emulsifying, solubilizing, and dispersing agents for various pharmaceutical compositions and preparations of biological materials. Pharmaceutically acceptable surfactants like polysorbates (e.g. polysorbates 20 or 80); polyoxamers (e.g. poloxamer 188); Triton; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, paImidopropyl-, or isostearamidopropyl-betaine (e.g. lauroamidopropyl); myristamidopropyl-, paImidopropyl-, or isostearamidopropyl-dimethylamine; sodium methyl cocoyl-, or disodium methyl oleyl-taurate; and the MONAQUA™ series (Mona Industries, Inc., Paterson, N.J.), polyethyl glycol, polypropyl glycol, and copolymers of ethylene and propylene glycol (e.g. PLURONICS™, PF68 etc), can optionally be added to the formulations of the disclosure to reduce aggregation. In one embodiment, a formulation of the disclosure comprises Polysorbate 20, Polysorbate 40, Polysorbate 60, or Polysorbate 80. Surfactants are particularly useful if a pump or plastic container is used to administer the formulation. The presence of a pharmaceutically acceptable surfactant mitigates the propensity for the protein to aggregate. In a specific embodiment, the formulations of the disclosure comprise a polysorbate which is at a concentration ranging from between about 0.001% to about 1%, or about 0.001% to about 0.1%, or about 0.01% to about 0.1%. In other specific embodiments, the formulations of the disclosure comprise a polysorbate which is at a concentration of 0.001%, or 0.002%, or 0.003%, or 0.004%, or 0.005%, or 0.006%, or 0.007%, or 0.008%, or 0.009%, or 0.01%, or 0.015%, or 0.02%. In another specific embodiment, the polysorbate is polysorbate-80. In a specific embodiment, the formulations of the disclosure comprise a polysorbate which is at a concentration ranging from between 0.001% to 1%, or 0.001% to 0.1%, or 0.01% to 0.1%. In other specific embodiments, the formulations of the disclosure comprise a polysorbate which is at a concentration of 0.001%, or 0.002%, or 0.003%, or 0.004%, or 0.005%, or 0.006%, or 0.007%, or 0.008%, or 0.009%, or 0.01%, or 0.015%, or 0.02%. In another specific embodiment, the polysorbate is polysorbate-80.

In a specific embodiment, a formulation of the disclosure comprises at least about 0.001%, at least about 0.002%, at least about 0.005%, at least about 0.01%, at least about 0.02%, at least about 0.05%, at least about 0, 1%, at least about 0.2%, or at least about 0.5% Polysorbate 80. In another embodiment, a formulation of the disclosure comprises between about 0.001% and about 0.5%, between about 0.001% and about 0.2%, between about 0.001% and about 0.1%, between about 0.001% and about 0.05%, between about 0.002% and about 0.5%, between about 0.002% and about 0.2%, between about 0.002% and about 0.1%, between about 0.002% and about 0.05%, between about 0.005% and about 0.5%, between about 0.005% and about 0.2%, between about 0.005% and about 0.1%, between about 0.005% and about 0.05%, between about 0.01% and about 0.5%, between about 0.01% and about 0.2%, between about 0.01% and about 0.1%, or between about 0.01% and about 0.05% Polysorbate 80. In a further embodiment, a formulation of the disclosure comprises about 0.001%, about 0.002%, about 0.005%, about 0.01%, about 0.02%, about 0.05%, about 0.1%, about 0.2%, and about 0.5% Polysorbate 80. In a specific embodiment, a formulation of the disclosure comprises about 0.005% Polysorbate 80. In a specific embodiment, a formulation of the disclosure comprises about 0.025% Polysorbate 80. In a specific embodiment, a formulation of the disclosure comprises about 0.02% Polysorbate 80. In a specific embodiment, a formulation of the disclosure comprises about 0.1% Polysorbate 80.

In certain embodiments, the formulations of the disclosure may optionally further comprise other common excipients and/or additives including, but not limited to, diluents, binders, stabilizers, lipophilic solvents, preservatives, adjuvants, or the like. Pharmaceutically acceptable excipients and/or additives may be used in the formulations of the disclosure. Commonly used excipients/additives, such as pharmaceutically acceptable chelators (for example, but not limited to, EDTA, DTPA or EGTA) can optionally be added to the formulations of the disclosure to reduce aggregation. These additives are particularly useful if a pump or plastic container is used to administer the formulation.

Preservatives, such as phenol, m-cresol, p-cresol, o-cresol, chlorocresol, benzyl alcohol, phenylmercuric nitrite, phenoxyethanol, formaldehyde, chlorobutanol, magnesium chloride (for example, but not limited to, hexahydrate), alkylparaben (methyl, ethyl, propyl, butyl and the like), benzalkonium chloride, benzethonium chloride, sodium dehydroacetate and thimerosal, or mixtures thereof can optionally be added to the formulations of the disclosure at any suitable concentration such as between about 0.001% to about 5%, or any range or value therein. The concentration of preservative used in the formulations of the disclosure is a concentration sufficient to yield a microbial effect. Such concentrations are dependent on the preservative selected and are readily determined by the skilled artisan.

Other contemplated excipients/additives, which may be utilized in the formulations of the disclosure include, for example, flavoring agents, antimicrobial agents, sweeteners, antioxidants, antistatic agents, lipids such as phospholipids or fatty acids, steroids such as cholesterol, protein excipients such as serum albumin (human serum albumin (HSA), recombinant human albumin (rHA)), gelatin, casein, salt-forming counterions such as sodium and the like. These and additional known pharmaceutical excipients and/or additives suitable for use in the formulations of the disclosure are known in the art, e.g., as listed in “Remington: The Science & Practice of Pharmacy”, 21st ed., Lippincott Williams & Wilkins, (2005), and in the “Physician's Desk Reference”, 60th ed., Medical Economics, Montvale, N.J. (2005). Pharmaceutically acceptable carriers can be routinely selected that are suitable for the mode of administration, solubility and/or stability of anti-PAI-1 antibody, as well known those in the art or as described herein.

It will be understood by one skilled in the art that the formulations of the disclosure may be isotonic with human blood, wherein the formulations of the disclosure have essentially the same osmotic pressure as human blood. Such isotonic formulations will generally have an osmotic pressure from about 250 mOSm to about 350 mOSm. Isotonicity can be measured by, for example, using a vapor pressure or ice-freezing type osmometer. Tonicity of a formulation is adjusted by the use of tonicity modifiers. “Tonicity modifiers” are those pharmaceutically acceptable inert substances that can be added to the formulation to provide an isotonity of the formulation. Tonicity modifiers suitable for this disclosure include, but are not limited to, saccharides, salts and amino acids.

In certain embodiments, the formulations of the present disclosure have an osmotic pressure from about 100 mOSm to about 1200 mOSm, or from about 200 mOSm to about 1000 mOSm, or from about 200 mOSm to about 800 mOSm, or from about 200 mOSm to about 600 mOSm, or from about 250 mOSm to about 500 mOSm, or from about 250 mOSm to about 400 mOSm, or from about 250 mOSm to about 350 mOSm.

The concentration of any one component or any combination of various components, of the formulations of the disclosure is adjusted to achieve the desired tonicity of the final formulation. For example, the ratio of the carbohydrate excipient to antibody may be adjusted according to methods known in the art (e.g., U.S. Pat. No. 6,685,940). In certain embodiments, the molar ratio of the carbohydrate excipient to antibody may be from about 100 moles to about 1000 moles of carbohydrate excipient to about 1 mole of antibody, or from about 200 moles to about 6000 moles of carbohydrate excipient to about 1 mole of antibody, or from about 100 moles to about 510 moles of carbohydrate excipient to about 1 mole of antibody, or from about 100 moles to about 600 moles of carbohydrate excipient to about 1 mole of antibody.

The desired isotonicity of the final formulation may also be achieved by adjusting the salt concentration of the formulations. Pharmaceutically acceptable salts and those suitable for this disclosure as tonicity modifiers include, but are not limited to, sodium chloride, sodium succinate, sodium sulfate, potassuim chloride, magnesium chloride, magnesium sulfate, and calcium chloride. In specific embodiments, formulations of the disclosures comprise NaCl, MgCl₂, and/or CaCl₂. In one embodiment, concentration of NaCl is between about 75 mM and about 150 mM. In another embodiment, concentration of MgCl₂ is between about 1 mM and about 100 mM. Pharmaceutically acceptable amino acids including those suitable for this disclosure as tonicity modifiers include, but are not limited to, proline, alanine, L-arginine, asparagine, L-aspartic acid, glycine, serine, lysine, and histidine.

In one embodiment the formulations of the disclosure are pyrogen-free formulations which are substantially free of endotoxins and/or related pyrogenic substances. Endotoxins include toxins that are confined inside a microorganism and are released only when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, even low amounts of endotoxins must be removed from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). When therapeutic proteins are administered in amounts of several hundred or thousand milligrams per kilogram body weight, as can be the case with antibodies, even trace amounts of harmful and dangerous endotoxin must be removed. In certain specific embodiments, the endotoxin and pyrogen levels in the composition are less then 10 EU/mg, or less then 5 EU/mg, or less then 1 EU/mg, or less then 0.1 EU/mg, or less then 0.01 EU/mg, or less then 0.001 EU/mg.

When used for in vivo administration, the formulations of the disclosure should be sterile. The formulations of the disclosure may be sterilized by various sterilization methods, including sterile filtration, radiation, etc. In one embodiment, the antibody formulation is filter-sterilized with a presterilized 0.22-micron filter. Sterile compositions for injection can be formulated according to conventional pharmaceutical practice as described in “Remington: The Science & Practice of Pharmacy”, 21st ed., Lippincott Williams & Wilkins, (2005). Formulations comprising antibodies, such as those disclosed herein, ordinarily will be stored in lyophilized form or in solution. It is contemplated that sterile compositions comprising antibodies are placed into a container having a sterile access port, for example, an intravenous solution bag or vial having an adapter that allows retrieval of the formulation, such as a stopper pierceable by a hypodermic injection needle. In one embodiment, a composition of the disclosure is provided as a pre-filled syringe.

In one embodiment, a formulation of the disclosure is a lyophilized formulation. The term “lyophilized” or “freeze-dried” includes a state of a substance that has been subjected to a drying procedure such as lyophilization, where at least 50% of moisture has been removed.

The phrase “bulking agent” includes a compound that is pharmaceutically acceptable and that adds bulk to a lyo cake. Bulking agents known to the art include, for example, carbohydrates, including simple sugars such as dextrose, ribose, fructose and the like, alcohol sugars such as mannitol, inositol and sorbitol, disaccharides including trehalose, sucrose and lactose, naturally occurring polymers such as starch, dextrans, chitosan, hyaluronate, proteins (e.g., gelatin and serum albumin), glycogen, and synthetic monomers and polymers.

A “lyoprotectant” is a molecule which, when combined with a protein of interest (such as an anti-PAI-1 antibody), significantly prevents or reduces chemical and/or physical instability of the protein upon lyophilization and subsequent storage. Lyoprotectants include, but are not limited to, sugars and their corresponding sugar alchohols; an amino acid such as monosodium glutamate or histidine; a methylamine such as betaine; a lyotropic salt such as magnesium sulfate; a polyol such as trihydric or higher molecular weight sugar alcohols, e.g. glycerin, dextran, erythritol, glycerol, arabitol, xylitol, sorbitol, and mannitol; propylene glycol; polyethylene glycol; PLURONICS™; and combinations thereof. Additional examples of lyoprotectants include, but are not limited to, glycerin and gelatin, and the sugars mellibiose, melezitose, raffinose, mannotriose and stachyose. Examples of reducing sugars include, but are not limited to, glucose, maltose, lactose, maltulose, iso-maltulose and lactulose. Examples of non-reducing sugars include, but are not limited to, non-reducing glycosides of polyhydroxy compounds selected from sugar alcohols and other straight chain polyalcohols. Examples of sugar alcohols include, but are not limited to, monoglycosides, compounds obtained by reduction of disaccharides such as lactose, maltose, lactulose and maltulose. The glycosidic side group can be either glucosidic or galactosidic. Additional examples of sugar alcohols include, but are not limited to, glucitol, maltitol, lactitol and iso-maltulose. In specific embodiments, trehalose or sucrose is used as a lyoprotectant.

The lyoprotectant is added to the pre-lyophilized formulation in a “lyoprotecting amount” which means that, following lyophilization of the protein in the presence of the lyoprotecting amount of the lyoprotectant, the protein essentially retains its physical and chemical stability and integrity upon lyophilization and storage.

In one embodiment, the molar ratio of a lyoprotectant (e.g., trehalose) and anti-PAI-1 antibody molecules of a formulation of the disclosure is at least about 10, at least about 50, at least about 100, at least about 200, or at least about 300. In another embodiment, the molar ratio of a lyoprotectant (e.g., trehalose) and anti-PAI-1 antibody molecules of a formulation of the disclosure is about 1, is about 2, is about 5, is about 10, about 50, about 100, about 200, or about 300.

A “reconstituted” formulation is one which has been prepared by dissolving a lyophilized antibody formulation in a diluent such that the antibody is dispersed in the reconstituted formulation. The reconstituted formulation is suitable for administration (e.g. parenteral administration) to a patient to be treated with the anti-PAI-1 antibody and, in certain embodiments of the disclosure, may be one which is suitable for intravenous administration.

The “diluent” of interest herein is one which is pharmaceutically acceptable (safe and non-toxic for administration to a human) and is useful for the preparation of a liquid formulation, such as a formulation reconstituted after lyophilization. In some embodiments, diluents include, but are not limited to, sterile water, bacteriostatic water for injection (BWFI), a pH buffered solution (e.g. phosphate-buffered saline), sterile saline solution, Ringer's solution or dextrose solution. In an alternative embodiment, diluents can include aqueous solutions of salts and/or buffers.

In certain embodiments, a formulation of the disclosure is a lyophilized formulation comprising an anti-PAI-1 antibody of the disclosure, wherein at least about 90%, at least about 95%, at least about 97%, at least about 98%, or at least about 99% of said antibody may be recovered from a vial upon shaking said vial for 4 hours at a speed of 400 shakes per minute wherein said vial is filled to half of its volume with said formulation. In another embodiment, a formulation of the disclosure is a lyophilized formulation comprising an anti-PAI-1 antibody of the disclosure, wherein at least about 90%, at least about 95%, at least about 97%, at least about 98%, or at least about 99% of said antibody may be recovered from a vial upon subjecting the formulation to three freeze/thaw cycles wherein said vial is filled to half of its volume with said formulation. In a further embodiment, a formulation of the disclosure is a lyophilized formulation comprising an anti-PAI-1 antibody of the disclosure, wherein at least about 90%, at least about 95%, at least about 97%, at least about 98%, or at least about 99% of said antibody may be recovered by reconstituting a lyophilized cake generated from said formulation.

In certain embodiments, a lyophilized formulation of the disclosure comprises anti-PAI-1 antibody molecules of the disclosure, wherein at least about 90%, at least about 95%, at least about 97%, at least about 98%, or at least about 99% of said antibody is recovered by reconstituting said lyophilized formulation upon storage at about 40° C. for at least about 1 week, at least about 2 weeks, at least about 3 weeks, at least about 4 weeks, at least about 5 weeks, or at least about 6 weeks. In one embodiment, a lyophilized formulation of the disclosure comprises anti-PAI-1 antibody molecules of the disclosure, wherein at least about 90%, at least about 95%, at least about 97%, at least about 98%, or at least about 99% of said antibody is recovered by reconstituting said lyophilized formulation upon storage at about 40° C. for at least about 1 month, at least about 2 months, at least about 3 months, at least about 4 months, at least about 5 months, or at least about 6 months.

In certain embodiments, a lyophilized formulation of the disclosure comprises anti-PAI-1 antibody molecules of the disclosure, wherein at least about 90%, at least about 95%, at least about 97%, at least about 98%, or at least about 99% of said antibody is recovered by reconstituting said lyophilized formulation upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a lyophilized formulation of the disclosure comprises anti-PAI-1 antibody molecules of the disclosure, wherein at least about 90%, at least about 95%, at least about 97%, at least about 98%, or at least about 99% of said antibody is recovered by reconstituting said lyophilized formulation upon storage at about 5° C. for at least about 1 year, at least about 2 years, at least about 3 years, at least about 4 years, or at least about 5 years.

In one embodiment, a reconstituted liquid formulation of the disclosure comprises an anti-PAI-1 antibody of the disclosure at the same concentration as the pre-lyophilized liquid formulation.

In one embodiment, a reconstituted liquid formulation of the disclosure comprises an anti-PAI-1 antibody of the disclosure at a higher concentration than the pre-lyophilized liquid formulation. In specific embodiments, a reconstituted liquid formulation of the disclosure comprises about 2 fold, about 3 fold, about 4 fold, about 5 fold, about 6 fold, about 7 fold, about 8 fold, about 9 fold, about 10 fold, about 15 fold, about 20 fold, about 30 fold, about 40 fold higher concentration of an anti-PAI-1 antibody of the disclosure than the pre-lyophilized liquid formulation.

In another embodiment, alternatively a reconstituted liquid formulation of the disclosure comprises an anti-PAI-1 antibody of the disclosure at a lower concentration than the pre-lyophilized liquid formulation. In specific embodiments, a reconstituted liquid formulation of the disclosure comprises about 2 fold, about 3 fold, about 4 fold, about 5 fold, about 6 fold, about 7 fold, about 8 fold, about 9 fold, about 10 fold, about 15 fold, about 20 fold, about 30 fold, about 40 fold lower concentration of an anti-PAI-1 antibody of the disclosure than the pre-lyophilized liquid formulation.

In certain embodiments, reconstituted formulations of the disclosure comprise (or consists of as the aggregate fraction) a particle profile of less than about 3.4 E+5 particles/ml of diameter 2-4 μm, less than about 4.0 E+4 particles/ml of diameter 4-10 μm, less than about 4.2 E+3 particles/ml of diameter 10-20 μm, less than about 5.0 E+2 particles/ml of diameter 20-30 μm, less than about 7.5 E+1 particles/ml of diameter 30-40 μm, and less than about 9.4 particles/ml of diameter 40-60 μm as determined by a particle multisizer. In certain embodiments, reconstituted formulations of the disclosure contain no detectable particles greater than 40 μm, or greater than 30 μm.

In certain embodiments, after storage for about 1 hour, about 2 hours, about 3 hours, about 4 hours, about 5 hours, about 6 hours, about 7 hours, about 8 hours, about 9 hours, about 12 hours, about 15 hours, about 18 hours, or about 24 hours reconstituted liquid formulations of the disclosure comprise (or consists of as the aggregate fraction) a particle profile of less than about 3.4 E+5 particles/ml of diameter 2-4 μm, less than about 4.0 E+4 particles/ml of diameter 4-10 μm, less than about 4.2 E+3 particles/ml of diameter 10-20 μm, less than about 5.0 E+2 particles/ml of diameter 20-30 μm, less than about 7.5 E+1 particles/ml of diameter 30-40 μm, and less than about 9.4 particles/ml of diameter 40-60 μm as determined by a particle multisizer. In certain embodiments, liquid formulations of the disclosure contain no detectable particles greater than 40 μm, or greater than 30 μm.

In one embodiment, a pharamceutical formulation of the disclosure is a stable formulation. In certain embodiments, a pharmaceutical composition of the disclosure is stable at 4° C. In certain embodiments, a pharmaceutical composition of the disclosure is stable at room temperature.

The terms “stability” and “stable” as used herein in the context of a formulation comprising an anti-PAI-1 antibody of the disclosure refer to the resistance of the antibody in the formulation to aggregation, degradation or fragmentation under given manufacture, preparation, transportation and storage conditions. The “stable” formulations of the disclosure retain biological activity under given manufacture, preparation, transportation and storage conditions. The stability of the anti-PAI-1 antibody can be assessed by degrees of aggregation, degradation or fragmentation, as measured by HPSEC, static light scattering (SLS), Fourier Transform Infrared Spectroscopy (FTIR), circular dichroism (CD), urea unfolding techniques, intrinsic tryptophan fluorescence, differential scanning calorimetry, and/or ANS binding techniques, compared to a reference formulation. For example, a reference formulation may be a reference standard frozen at −70° C. consisting of 10 mg/ml of an anti-PAI-1 antibody of the disclosure in PBS.

In specific embodiments, the pharmaceutical compositions include, but are not limited to:

(a) a sterile liquid formulation comprising 100 mg/ml of antibody, 25 mM histidine, 1.6 mM glycine at pH 6.0;
(b) a sterile liquid formulation comprising 100 mg/ml of antibody and 25 mM histidine-HCl at pH 6.0;
(c) a sterile liquid formulation comprising 25 mg/ml of antibody, 20 mM Citric acid, 100 mM NaCl, 1.5% mannitol, 50 μl DTPA, and 0.02% Polysorbate 80 at pH 6.0;
(d) a sterile liquid formulation comprising 100 mg/ml of antibody, 25 mM histidine, 8% trehalose, and 0.02% Polysorbate 80 at pH 6.0;
(e) a sterile liquid formulation comprising 20 mg/ml of antibody, 10 mM Histidine, 2.35% (w/v) Lysine-HCl, and 0.02% Polysorbate 80 (w/v) at pH 6.0;
(f) a sterile liquid formulation comprising 5 mg/ml of antibody, 10 mM Sodium citrate buffer, NaCl (0.15M) and Tween 80 (0.02%) at pH 6.0;
(g) a sterile liquid formulation comprising 100 mg/ml of antibody, 10 mM histidine and 150 mM NaCl at pH 6.0;
(h) a sterile liquid formulation comprising 100 mg/ml of antibody, 25 mM histidine, 1.6 mM glycine, 3% mannitol at pH 6.0;
(i) a sterile liquid formulation comprising 10 mg/ml or 100 mg/ml of antibody, 10 mM histidine-HCl, 105 mM NaCl, 0.005% polysorbate 80 at pH 6.0;
(j) a sterile liquid formulation comprising 100 mg/ml of antibody, 10 mM histidine-HCl, 0.005% polysorbate 80 at pH 6.0;
(k) a sterile liquid formulation comprising 100 mg/ml of antibody, 6% trehalose, 2% arginine, 0.025% polysorbate, 10m M histidine at pH 6.0;
(l) a sterile liquid formulation comprising 100 mg/ml of antibody, 20 mM citric acid, 200 mM lysine, 15% trehalose, 0.1% polysorbate 80 at pH 6.0;
(m) a sterile liquid formulation comprising 100 mg/ml of antibody, 20 mM citric acid, 199 mM NaCl, 1.5% mannitol, 50 μl DTPA, 0.02% polysorbate 80 at pH 6.0

The liquid formulations of the present disclosure exhibit stability at the temperature ranges of 38° C. to 42° C. for at least 60 days and, in some embodiments, not more than 120 days, of 20° C. to 24° C. for at least 1 year, of 2° C. to 8° C. (in particular, at 4° C.) for at least 3 years, at least 4 years, or at least 5 years and at −20° C. for at least 3 years, at least 4 years, or at least 5 years, as assessed by high performance size exclusion chromatography (HPSEC). Namely, the liquid formulations of the present disclosure have low to undetectable levels of aggregation and/or fragmentation, as defined herein, after the storage for the defined periods as set forth above. Preferably, no more than 5%, no more than 4%, no more than 3%, no more than 2%, no more than 1%, and most preferably no more than 0.5%, of anti-PAI-1 antibody forms an aggregate as measured by HPSEC, after the storage for the defined periods as set forth above. Furthermore, liquid formulations of the present disclosure exhibit almost no loss in biological activity(ies) of anti-PAI-1 antibodies during the prolonged storage under the condition described above, as assessed by various immunological assays including, for example, enzyme-linked immunosorbent assay (ELISA) and radioimmunoassay to measure the PAI-1 antigen-binding ability of anti-PAI-1 antibody. The liquid formulations of the present disclosure retain after the storage for the above-defined periods more than 80%, more than 85%, more than 90%, more than 95%, more than 98%, more than 99%, or more than 99.5% of the initial biological activity(ies) prior to the storage.

Therapeutic compositions of the present disclosure may be formulated for a particular dosage. Dosage regimens may be adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the disclosure are dictated by and directly dependent on (a) the unique characteristics of the anti-PAI-1 antibody and the particular therapeutic effect to be achieved, and (b) the limitations inherent in the art of compounding such an anti-PAI-1 antibody for the treatment of sensitivity in individuals.

Therapeutic compositions of the present disclosure can be formulated for particular routes of administration, such as oral, nasal, pulmonary, topical (including buccal and sublingual), rectal, vaginal and/or parenteral administration. The formulations may conveniently be presented in unit dosage form and may be prepared by any methods known in the art of pharmacy. The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. By way of example, in certain embodiments, the antibodies (including antibody fragments) are formulated for intravenous administration. In certain other embodiments, the antibodies (including antibody fragments) are formulated for local delivery to the lung or kidney, for example, for delivery via inhalation, for intranasal delivery, or for delivery via a dialysis port.

Formulations of the present disclosure which are suitable for topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required (U.S. Pat. Nos. 7,378,110; 7,258,873; 7,135,180; US Publication No. 2004-0042972; and 2004-0042971).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Actual dosage levels of the active ingredients in the pharmaceutical compositions of the present disclosure may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present disclosure employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

In certain embodiments, antibodies of the disclosure can be formulated to ensure proper distribution in vivo. For example, the blood-brain barrier (BBB) excludes many highly hydrophilic compounds. To ensure that the therapeutic compounds of the disclosure can cross the BBB (if desired), they can be formulated, for example, in liposomes. For methods of manufacturing liposomes, see, e.g., U.S. Pat. Nos. 4,522,811; 5,374,548; 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134), different species of which may comprise the formulations of the disclosures, as well as components of the invented molecules; p120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273. In one embodiment of the disclosure, the therapeutic compounds of the disclosure are formulated in liposomes; in another embodiment, the liposomes include a targeting moiety. In another embodiment, the therapeutic compounds in the liposomes are delivered by bolus injection to a site proximal to the desired area. When administered in this manner, the composition must be fluid to the extent that easy syringability exists. It must be stable under the conditions of manufacture and storage and may be preserved against the contaminating action of microorganisms such as bacteria and fungi. Additionally or alternatively, the antibodies of the disclosure may be delivered locally to the brain to mitigate the risk that the blood brain barrier slows effective delivery.

In certain embodiments, the therapeutic antibody compositions may be administered with medical devices known in the art. For example, in certain embodiments an antibody or antibody fragment is administered locally via a catheter, stent, wire, or the like. For example, in one embodiment, a therapeutic composition of the disclosure can be administered with a needleless hypodermic injection device, such as the devices disclosed in U.S. Pat. Nos. 5,399,163; 5,383,851; 5,312,335; 5,064,413; 4,941,880; 4,790,824; 4,596,556. Examples of well-known implants and modules useful in the present disclosure include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

The efficient dosages and the dosage regimens for the antibodies of the disclosure depend on the disease or condition to be treated and can be determined by the persons skilled in the art.

A “therapeutically effective dosage” for treating a disease or condition caused or exacerbated by fibrosis may, for example, decrease fibrosis, decrease inflammation, improve lung function, improve kidney function, decrease/prevent/delay need for dialysis, delay or prevent the need for kidney or lung transplantation, decrease/reduce/eliminate reliance on other medications, etc. One of ordinary skill in the art would be able to determine such amounts based on such factors as the subject's size, the severity of the subject's symptoms, and the particular composition or route of administration selected. It is recognized that antibodies of the disclosure may, in certain embodiments, be administered in multiple doses over a period of time.

### G. Articles of Manufacture and Kits

This section of the specification describes various exemplary kits and packages comprising anti-PAI-1 antibodies (including antibody fragments) of the present disclosure. It should be understood that any of the anti-PAI-1 antibodies and antibody fragments described herein, including antibodies and antibody fragments having any one or more of the structural and functional features described in detail throughout the application, may be packaged, sold, and/or used as part of a kit or package, as described in this section. When various kits and packages are described in this section as including an antibody, it is understood that such an antibody may be an antibody or an antibody fragment having any one or more of the characteristics of the anti-PAI-1 antibodies and antibody fragments described herein. The disclosure contemplates all combinations of any of the aspects and embodiments of the disclosure.

The disclosure provides a pharmaceutical pack or kit comprising one or more containers filled with a liquid formulation or lyophilized formulation of the disclosure (e.g., a formulation comprising an anti-PAI-1 antibody or antibody fragment of the present disclosure). In one embodiment, a container filled with a liquid formulation of the disclosure is a pre-filled syringe. In one embodiment, the formulations of the disclosure comprise anti-PAI-1 antibodies recombinantly fused or chemically conjugated to another moiety, including but not limited to, a heterologous protein, a heterologous polypeptide, a heterologous peptide, a large molecule, a small molecule, a marker sequence, a diagnostic or detectable agent, a therapeutic moiety, a drug moiety, a radioactive metal ion, a second antibody, and a solid support. In a specific embodiment, the formulations of the disclosure are formulated in single dose vials as a sterile liquid. The formulations of the disclosure may be supplied in 3 cc USP Type I borosilicate amber vials (West Pharmaceutical Serices—Part No. 6800-0675) with a target volume of 1.2 mL. Optionally associated with such container(s) can be a notice in the form prescribed by a governmental agency regulating the manufacture, use or sale of pharmaceuticals or biological products, which notice reflects approval by the agency of manufacture, use or sale for human administration. In another embodiment, a formulation of the disclosure may be supplied in a pre-filled syringe.

In one embodiment, a container filled with a liquid formulation of the disclosure is a pre-filled syringe. Any pre-filled syringe known to one of skill in the art may be used in combination with a liquid formulation of the disclosure. Pre-filled syringes that may be used are described in, for example, but not limited to, PCT Publications WO05032627, WO08094984, WO9945985, WO03077976, U.S. Pat. Nos. 6,792,743, 5,607,400, 5,893,842, 7,081,107, 7,041,087, 5,989,227, 6,807,797, 6,142,976, 5,899,889, US Patent Publications US20070161961A1, US20050075611A1, US20070092487A1, US20040267194A1, US20060129108A1. Pre-filled syringes may be made of various materials. In one embodiment a pre-filled syringe is a glass syringe. In another embodiment a pre-filled syringe is a plastic syringe. One of skill in the art understands that the nature and/or quality of the materials used for manufacturing the syringe may influence the stability of a protein formulation stored in the syringe. For example, it is understood that silicon based lubricants deposited on the inside surface of the syringe chamber may affect particle formation in the protein formulation. In one embodiment, a pre-filled syringe comprises a silicone based lubricant. In one embodiment, a pre-filled syringe comprises baked on silicone. In another embodiment, a pre-filled syringe is free from silicone based lubricants. One of skill in the art also understands that small amounts of contaminating elements leaching into the formulation from the syringe barrel, syringe tip cap, plunger or stopper may also influence stability of the formulkation. For example, it is understood that tungsten introduced during the manufacturing process may adversely affect formulation stability. In one embodiment, a pre-filled syringe may comprise tungsten at a level above 500 ppb. In another embodiment, a pre-filled syringe is a low tungsten syringe. In another embodiment, a pre-filled syringe may comprise tungsten at a level between about 500 ppb and about 10 ppb, between about 400 ppb and about 10 ppb, between about 300 ppb and about 10 ppb, between about 200 ppb and about 10 ppb, between about 100 ppb and about 10 ppb, between about 50 ppb and about 10 ppb, between about 25 ppb and about 10 ppb.

In certain embodiments, kits comprising anti-PAI-1 antibodies are also provided that are useful for various purposes, e.g., research and diagnostic including for purification or immunoprecipitation of PAI-1 from cells, detection of PAI-1 in vitro or in vivo, etc. For isolation and purification of PAI-1, the kit may contain an anti-PAI-1 antibody coupled to beads (e.g., sepharose beads). Kits may be provided which contain the antibodies for detection and quantitation of PAI-1 in vitro, e.g. in an ELISA or a Western blot. As with the article of manufacture, the kit comprises a container and a label or package insert on or associated with the container. The container holds a composition comprising at least one anti-PAI-1 antibody of the disclosure. Additional containers may be included that contain, e.g., diluents and buffers, control antibodies. The label or package insert may provide a description of the composition as well as instructions for the intended in vitro or diagnostic use.

The present disclosure also encompasses a finished packaged and labeled pharmaceutical product. This article of manufacture includes the appropriate unit dosage form in an appropriate vessel or container such as a glass vial, pre-filled syringe or other container that is hermetically sealed. In one embodiment, the unit dosage form is provided as a sterile particulate free solution comprising an anti-PAI-1 antibody that is suitable for parenteral administration. In another embodiment, the unit dosage form is provided as a sterile lyophilized powder comprising an anti-PAI-1 antibody that is suitable for reconstitution.

In one embodiment, the unit dosage form is suitable for intravenous, intramuscular, intranasal, oral, topical or subcutaneous delivery. Thus, the disclosure encompasses sterile solutions suitable for each delivery route. The disclosure further encompasses sterile lyophilized powders that are suitable for reconstitution.

As with any pharmaceutical product, the packaging material and container are designed to protect the stability of the product during storage and shipment. Further, the products of the disclosure include instructions for use or other informational material that advise the physician, technician or patient on how to appropriately prevent or treat the disease or disorder in question, as well as how and how frequently to administer the pharmaceutical. In other words, the article of manufacture includes instruction means indicating or suggesting a dosing regimen including, but not limited to, actual doses, monitoring procedures, and other monitoring information.

Specifically, the disclosure provides an article of manufacture comprising packaging material, such as a box, bottle, tube, vial, container, pre-filled syringe, sprayer, insufflator, intravenous (i.v.) bag, envelope and the like; and at least one unit dosage form of a pharmaceutical agent contained within said packaging material, wherein said pharmaceutical agent comprises a liquid formulation containing an antibody. The packaging material includes instruction means which indicate how that said antibody can be used to prevent, treat and/or manage one or more symptoms associated with a disease or disorder.

### H. Dog and Cyno PAI-1

Proteins can be prepared using methods known in the art. For example, proteins can be prepared using recombinant technology or synthetically. Recombinantly producing a protein can be done by expressing a nucleic acid encoding the protein in a cell under conditions where the protein is made, and purifying the protein from the cell. Any suitable cell can be used. Exemplary cells include bacterial cells, yeast cells, insect cells, and mammalian cells (e.g., CHO cells, COS cells, etc.).

In certain embodiments, the disclosure provides a purified or isolated polypeptide, comprising the amino acid sequence of dog PAI-1 polypeptide, such as a polypeptide comprising the amino acid sequence of SEQ ID NO: A (e.g., dog PAI-1 inclusive of the leader sequence). In certain embodiments, the polypeptide consists of the amino acid sequence of SEQ ID NO: A. In certain embodiments, the polypeptide comprises or consists of the amino acid sequence of SEQ ID NO:A, in the absence of the leader sequence.

Dog PAI-1 amino acid sequence (leader sequence underlined)

(SEQ ID NO: A)
MGSSHHHHHHSSGLVPRGSHMSYSYQTRAAGLATDFGVKVFQEVARAS
KDRNMVFSPYGVASVLAMLQLTTAGETCRQIQEAMQFQIDEKGMAPAL
RQLYKELMGPWNKDEISTADAIFVQRDLKLVHGFMPHFFRLFRTTVKQ
VDFSEVERARFIVNDWVKRHTKGMIGNLLGRGAVDQLTRLMLVNALYF
NGQWKTPFPESGTHHRLFHKSDGSTVSVPMMAQTNKFNYTEFSTPSGH
YYDILELPYHGDTLSMFIAAPYEKEVPLSALTNILDAQLISQWKGNMT
RQLRLLVLPKFSLETEVNLRRPLENLGMTDMFRPNLADFSSLSNQEVL
YVSRALQKVKIEVNESGTVASSSTAIIVSARMAPEEIIMDRPFLFVVR
HNPTGTVLFMGQVMEP

In certain embodiments, the dog PAI-1 is glycosylated. Such glycosylation may occur in the cell used to produce the protein recombinantly, or such glycosylation may be performed synthetically after the protein is produced recombinantly. In certain embodiments, the dog PAI-1 is glycosylated in a manner that differs from the native glycosylation pattern of the naturally occurring polypeptide. In other words, the glycosylation pattern is different than that which occurs naturally when PAI-1 is produced in the canine body and/or in canine cells. In certain embodiments, the polypeptide is produced in non-canine cells.

In certain embodiments, the disclosure provides a purified or isolated polypeptide, comprising the amino acid sequence of cynomolgus PAI-1 polypeptide, such as a polypeptide comprising the amino acid sequence of SEQ ID NO: B (e.g., cyno PAI-1 inclusive of the leader sequence) or SEQ ID NO: C (e.g., cyno PAI-1 in the absence of the leader sequence). In certain embodiments, the polypeptide consists of the amino acid sequence of SEQ ID NO: B or SEQ ID NO: C.

Cynomolgus PAI-1 amino acid sequence (leader sequence underline)

(SEQ ID NO: B)
MQMSPALACLVLGLAFVFGEGSTVHHPPSYVAHLASDFGVRVFQQVAQ
ASKDRNVVFSPYGVASVLAMLQLTTGGETRQQIQAAMGFKIDDKGMAP
ALRHLYKELLGPWNKDEISTTDAIFVQRDLKLVQGFMPHFFRLFRSTV
KQVDFSEAERARFIINDWVKTHTKGMISDLLGKGAVDQLTRLVLVNAL
YFNGHWKTPFPDSSTHRRLFHKSDGSTVSVPMMAQTNKFNYTEFTTPD
GHYYDILELPYHGNTLSMFIAAPYEKQVPLSALTNILSAQLISHWKGN
MTRLPRLLVLPKFSLETEVDLRKPLENLGMTDMFRQFQADFTSLSNQE
PLHVAQALQKVKIEVNESGTVASSSTAVIVSARMAPEEIIMDRPFLFV
VRHNPTGTVLFMGQVMEP
(SEQ ID NO: C)
VHHPPSYVAHLASDFGVRVFQQVAQASKDRNVVFSPYGVASVLAMLQL
TTGGETRQQIQAAMGFKIDDKGMAPALRHLYKELLGPWNKDEISTTDA
IFVQRDLKLVQGFMPHFFRLFRSTVKQVDFSEAERARFIINDWVKTHT
KGMISDLLGKGAVDQLTRLVLVNALYFNGHWKTPFPDSSTHRRLFHKS
DGSTVSVPMMAQTNKFNYTEFTTPDGHYYDILELPYHGNTLSMFIAAP
YEKQVPLSALTNILSAQLISHWKGNMTRLPRLLVLPKFSLETEVDLRK
PLENLGMTDMFRQFQADFTSLSNQEPLHVAQALQKVKIEVNESGTVAS
SSTAVIVSARMAPEEIIMDRPFLFVVRHNPTGTVLFMGQVMEP

Cynomolgus PAI-1 cDNA sequence

ATGCAGATGTCTCCAGCCCTCGCCTGCCTAGTCCTGGGCCTGGCCTTC
GTCTTTGGTGAAGGGTCCACCGTGCACCATCCCCCATCCTACGTGGCC
CACCTGGCCTCAGACTTCGGGGTTAGGGTGTTTCAGCAGGTGGCGCAG
GCCTCCAAAGACCGCAACGTGGTTTTCTCACCCTATGGGGTGGCCTCG
GTGTTGGCCATGCTCCAGCTGACAACGGGAGGAGAAACCCGGCAGCAG
ATCCAAGCGGCCATGGGATTCAAGATTGATGACAAGGGCATGGCTCCC
GCCCTCCGGCATCTGTACAAGGAGCTCTTGGGGCCGTGGAACAAGGAT
GAGATCAGCACCACAGACGCGATCTTTGTCCAGCGGGATCTGAAGCTG
GTCCAGGGCTTCATGCCCCACTTCTTCAGGCTGTTCCGGAGTACGGTC
AAGCAGGTGGACTTTTCAGAGGCAGAGAGAGCCAGATTCATCATCAAC
GACTGGGTGAAGACACACACAAAAGGGATGATCAGTGACTTGCTTGGG
AAAGGAGCCGTGGACCAGCTGACACGGCTGGTGCTGGTGAACGCCCTC
TACTTCAACGGCCACTGGAAGACTCCCTTCCCCGACTCCAGCACCCAC
CGCCGCCTCTTCCACAAATCAGATGGCAGCACTGTCTCTGTGCCCATG
ATGGCTCAGACCAACAAGTTCAACTATACCGAGTTCACCACGCCCGAT
GGCCATTACTACGACATCCTGGAACTGCCCTACCACGGAAACACCCTC
AGCATGTTCATTGCTGCCCCTTATGAAAAGCAGGTGCCTCTCTCTGCC
CTCACCAACATTCTGAGTGCCCAGCTCATCAGCCACTGGAAAGGCAAC
ATGACCAGGCTGCCCCGCCTCCTGGTTCTGCCCAAGTTCTCCCTGGAG
ACTGAAGTTGACCTCAGGAAGCCCCTAGAGAACCTGGGAATGACCGAC
ATGTTCAGACAATTTCAGGCGGACTTCACGAGTCTTTCAAACCAAGAG
CCTCTCCACGTTGCGCAAGCGCTTCAGAAAGTGAAGATCGAGGTGAAC
GAGAGTGGCACGGTGGCCTCCTCATCCACAGCTGTCATAGTCTCAGCC
CGGATGGCGCCTGAGGAGATCATCATGGACAGACCCTTCCTCTTTGTG
GTCCGGCACAACCCCACAGGAACAGTCCTTTTCATGGGCCAAGTGATG
GAACCTTGA

In certain embodiments, the cyno PAI-1 is glycosylated. Such glycosylation may occur in the cell used to produce the protein recombinantly, or such glycosylation may be performed synthetically after the protein is produced recombinantly. In certain embodiments, the cyno PAI-1 is glycosylated in a manner that differs from the native glycosylation pattern of the naturally occurring polypeptide. In other words, the glycosylation pattern is different than that which occurs naturally when PAI-1 is produced in the cynomolgus body and/or in cynomolgus cells. In certain embodiments, the polypeptide is produced in non-cynomolgus cells. In certain embodiments, the polypeptide is produced in non-human cells and/or non-cynomolgus cells. In certain embodiments, the polypeptide is produced in cells other than cells from a non-human primate and/or human.

In another aspect, the disclosure provides a purified or isolated complex comprising any of the foregoing PAI-1 polypeptides and a vitronectin polypeptide. In certain embodiment, the complex consists of any of the foregoing PAI-1 polypeptides and a vitronectin polypeptide.

In certain embodiments, the PAI-1 polypeptide in the complex is a dog PAI-1 polypeptide having any of the sequence and glycosylation characteristics set forth above. In certain embodiments, the PAI-1 polypeptide in the complex is a cynomolgus PAI-1 polypeptide having any of the sequence and glycosylation characteristics set forth above. In certain embodiments, the vitronectin polypeptide is a dog, cyno, or human vitronectin polypeptide.

### EXAMPLES

The examples below are given so as to illustrate the practice of this disclsoure. The reagents employed in the examples are commercially available or can be prepared using commercially available instrumentation, methods, or reagents known in the art. The foregoing examples illustrate various aspects of the disclsoure and practice of the methods of the disclsoure. The examples are not intended to provide an exhaustive description of the many different embodiments of the disclsoure. Thus, although the forgoing disclsoure has been described in some detail by way of illustration and example for purposes of clarity of understanding, those of ordinary skill in the art will realize readily that many changes and modifications can be made thereto without departing from the spirit or scope of the appended claims. The examples are not intended to limit or define the entire scope of this disclsoure.

### Example 1

### Charactization of Anti-PAI-1 Antibodies

This example describes the characterisation of various anti-PAI-1. The desired characteristics were: potent human, cynomolgus and rat PAI-1 neutralising activity in a functional assay, selectivity for human PAI-1 over other circulating serpins, selectivity for active PAI-1 over latent, lack of inhibition of PAI-1 binding to vitronectin and confirmed species cross reactivity by competition ELISA.

Cynomolgus cross reactivity will enable safety studies to be carried out using cynomolgus monkey. Functional cross-reactivity against rat PAI-1 permits demonstration of efficacy in rodent disease models. Selectivity for PAI-1 over other circulating serpins, and lack of inhibition of PAI-1 binding to vitronectin, is desired in order to minimise any potential safety risks related to disruption of the varied functions associated with other serpin family members, and inhibition of other vitronectin-mediated functions for which PAI-1 has been implicated (for example, regulation of cell binding and cell migration). Selectivity for active PAI-1 over latent (inactive) avoids the sink of latent PAI-1 thought to represent the majority of circulating antigen (t_{1/2} for the irreversible active to latent transition is approximately 2 hours

A functional biochemical assay (chromogenic assay) was established in which the activity of human tPA in the presence of both human PAI-1 and anti-PAI-1 scFv's is detected by the cleavage of a chromogenic substrate. Assay formats included a part-rat version of this functional assay in which the activity of rat tPA in the presence of both rat PAI-1 and the purified scFv was measured, and a part-cynomolgus version of this assay in which the inhibition of the activity of cynomolgus PAI-1 on human tPA was measured.

To determine the effect of scFv on binding of PAI-1 to vitronectin, a DELFIA format competition assay involving human PAI-1 binding to immobilised human monomeric vitronectin was performed.

Screening

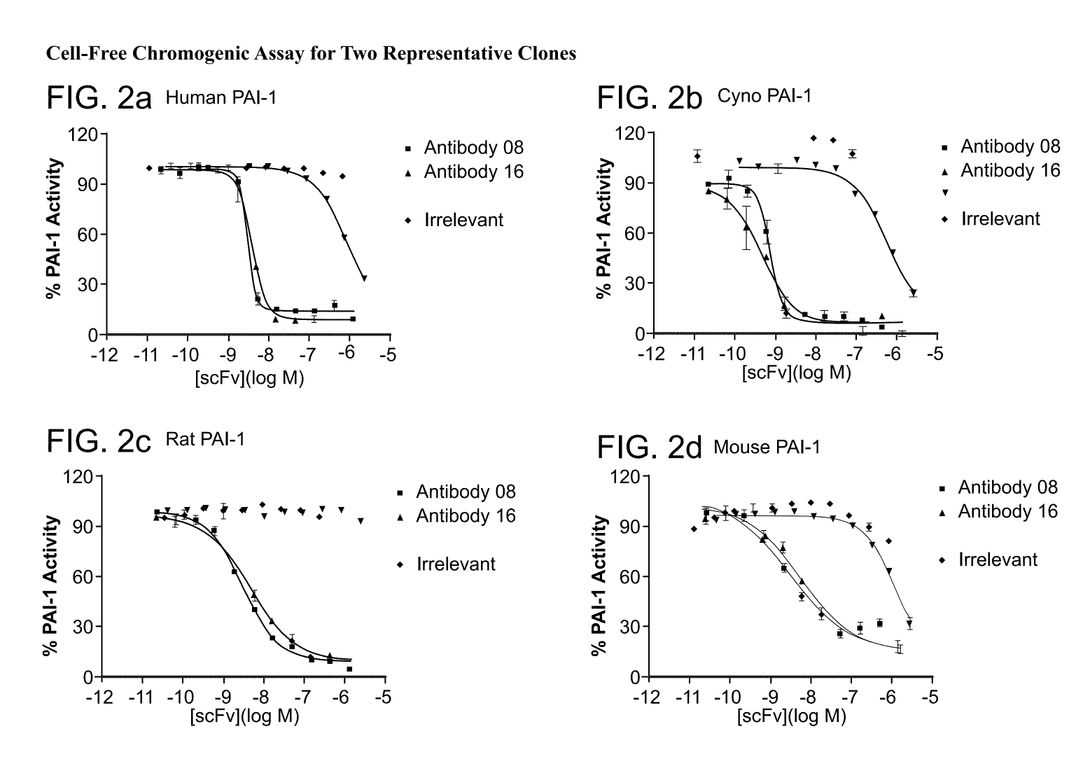

scFvs were tested in the human, cyno, rat and mouse cell-free chromogenic assays as described herein. Examples of data on IC₅₀ of antibodies 1 to 27 are shown in Table 3. Sequence information for the CDRs of antibodies 1 to 27, as well as the VH and VL, are provided in the Sequence Listing and tables. Data for two representative clones (Antibody 08 and Antibody 16) are shown in FIG. 2. IC₅₀ values measured for Antibody 08 scFv in human (FIG. 2a) and cyno (FIG. 2b) assays were 3.6 nM and 0.8 nM, respectively. IC₅₀ values for Antibody 16 scFv in the human (FIG. 2a) and cyno (FIG. 2b) assays under the chosen conditions were 3.6 nM and 0.8 nM, respectively. As shown in FIGS. 2c and 2d, IC₅₀ values in the rat and mouse assays were 3 nM and 2.6 nM, respectively, for Antibody 08 and 5.6 nM and 4.3 nM, respectively, for Antibody 16. Therefore, clones were equipotent on all species variants.

The above findings were confirmed with whole antibodies. The scFvs were converted to IgG and purified before testing again in cell-free chromogenic assays. The IgG IC₅₀ values were very similar to values for the corresponding scFv, e.g., Antibody 08 had IC₅₀ of 1.9 nM in the human assay, 0.8 nM in the cyno assay, 2 nM in the rat assay and 1.2 nM in the mouse assay.

In addition, the clones were tested in a dog cell-free chromogenic assay. The IC₅₀ for an IgG tested in this assay was 12.1 nM (i.e., within 10-fold ratio to potency on human PAI-1).

Further, the clones were tested on glycosylated active human PAI-1 produced by Chinese hamster ovary (CHO) cells (Stromqvist, M., et al. Protein Expr Purif, 1994. 5: p. 309). Exemplified by IC₅₀s of 1.7 nM for Antibody 1, 1.5 nM for Antibody 2, 1.3 nM for Antibody 08, 1.2 nM for Antibody 16, 1.6 nM for Antibody 20, and 2.6 nM for Antibody 27 (see Table 4), the potencies were nearly identical to those measured on the previously used bacterially-derived PAI-1 and therefore did not suggest any dependency on the glycosylation state of the molecule.

Most of the selected scFv and IgG have an IC₅₀ in the human chromogenic assay of <10 nM.

The variable heavy chains and the variable light chains of the antibodies shown in Table 3 were sequenced to determine their DNA sequences, and a lineup of the CDR sequences for antibodies 1-27 is depicted in Table 2. The complete sequence information for these anti-PAI-1 antibodies is provided in the Sequence Listing with nucleotide and amino acid sequences. Sequences of antibodies of the disclsoure are shown in the appended Sequence Listing, in which SEQ ID NOs correspond as shown in Table 16.

### Example 2

### Neutralisation Potency of Anti-PAI-1 Antibodies in the HT-1080 Plasminogen Activation Assay with Exogenous Human, Rat and Mouse PAI-1

Select scFVs were tested as IgG in the HT-1080 plasminogen activation assay. Using an adapted method of Osada et al. (1991), HT-1080 cells were plated (˜20000/well, 96 well format) and cultured for 20-24 h. The media was then aspirated, and cell monolayers were incubated with antibody in the presence of the chromogenic plasmin substrate S-2251 (0.2 mM) and 5 μg ml⁻¹ Lys-plasminogen in 200 μl of HBSS at 37° C. The increase in absorbance at 405 nm was then followed for up to 3 h. 15 nM rat or mouse PAI-1 (US Biological) instead of the human reagent were used to confirm cross-reactivity of the clones in this assay. Examples of data on IC50 (nM) of selected antibodies are shown in Table 5.

The HT-1080 assay was also used to assess the potency of germlined versions of the scFv clones. The amino acid sequences of their VHs and VLs were aligned to the known human germline sequences in the VBASE database (Tomlinson, I., VBASE. 1997, MRC Centre of Protein Engineering, Cambridge, UK), and the closest germline was identified by sequence similarity. For the VH domains, this corresponds to VH1-69 (DP-10), and for VL, Vkappal L12. There were between 1 and 5 changes in the frameworks of the VH domains and between 5 and 6 in the VL domains, all of which were reverted to the closest germline sequence by site-directed mutagenesis to match human antibodies. Only the Vernier residues (Foote, J., et al. J Mol Biol, 1992. 224: p. 487) mutated during the optimisation process were left unchanged. The resulting IgGs were retested in the human and rat HT-1080 plasminogen activation assays, and the potencies for the two versions of each clone were demonstrated to be very similar. A representative scFV gave results with IC₅₀ values of 7.4 nM for the non-germlined and 11.3 nM for the germlined variant. In addition, the potency of this clone in the rat assay was unaffected by the germlining. The IC₅₀s for non-germlined and germlined IgG were 17.3 nM and 19.6 nM, respectively. Therefore, none of the germline framework mutations in the VH and VL domains of these antibodies are essential for their potency on human or rodent PAI-1, and all could be reverted to the closest germline.

### Example 3

### Neutralisation Potency of Anti-PAI-1 Antibodies in the Human and Mouse Plasminogen Activation Assays with Endogenous PAI-1

In the plasminogen activation assays described in Example 2, recombinant PAI-1 from different species was added to HT-1080 cells at a defined concentration. This allowed comparative potency determination of the anti-PAI-1 antibodies on human and rodent PAI-1. In addition, it was also shown in the chromogenic assay that the antibodies inhibited glycosylated active human PAI-1 from eukaryotic cells as well as the bacterially derived molecule with similar IC₅₀s (see Example 1). In order to confirm all this data on native PAI-1, the scFV antibodies (Table 1?) were tested in plasminogen activation assays on endogenous rather than added recombinant PAI-1. To this end, transforming growth factor β₁ (TGFβ₁) was used to induce PAI-1 production in the human HT-1080 and Normal Human Lung Fibroblast (NHLF, Clonetics/Biowhittaker) as well as mouse fibroblast MLg (ECACC) cells. The protocol of the HT-1080 plasminogen activation assay was as described below and as used in Example 2, but with the following exceptions: NHLF cells were seeded at 1×10⁴ cells/well in 96-well flat-bottomed tissue culture assay plates (Costar), MLg cells at 2×10⁴ cells/well in 96-well flat-bottomed collagen I-coated tissue culture assay plates (Becton Dickinson). PAI-1 production was induced by culturing the cells for 20 hours in the presence of 100 pM recombinant human TGFβ₁ (R&D Systems).

In the HT-1080 assaya representative antibody had an IC₅₀ of 1.4 nM. The potency was further confirmed in the more disease-relevant fibroblast cell line NHLF. The IC₅₀ for a representative antibody was 3.7 nM. Finally, the functional cross-reactivity of the scFV clones shown on recombinant mouse PAI-1 added to human HT-1080 cells was confirmed in the MLg assay releasing both mouse uPA/tPA and mouse PAI-1. A representative antibody, for example, inhibited with an IC₅₀ of 0.6 nM.

### Example 4

### Characterization of PICK167_A01

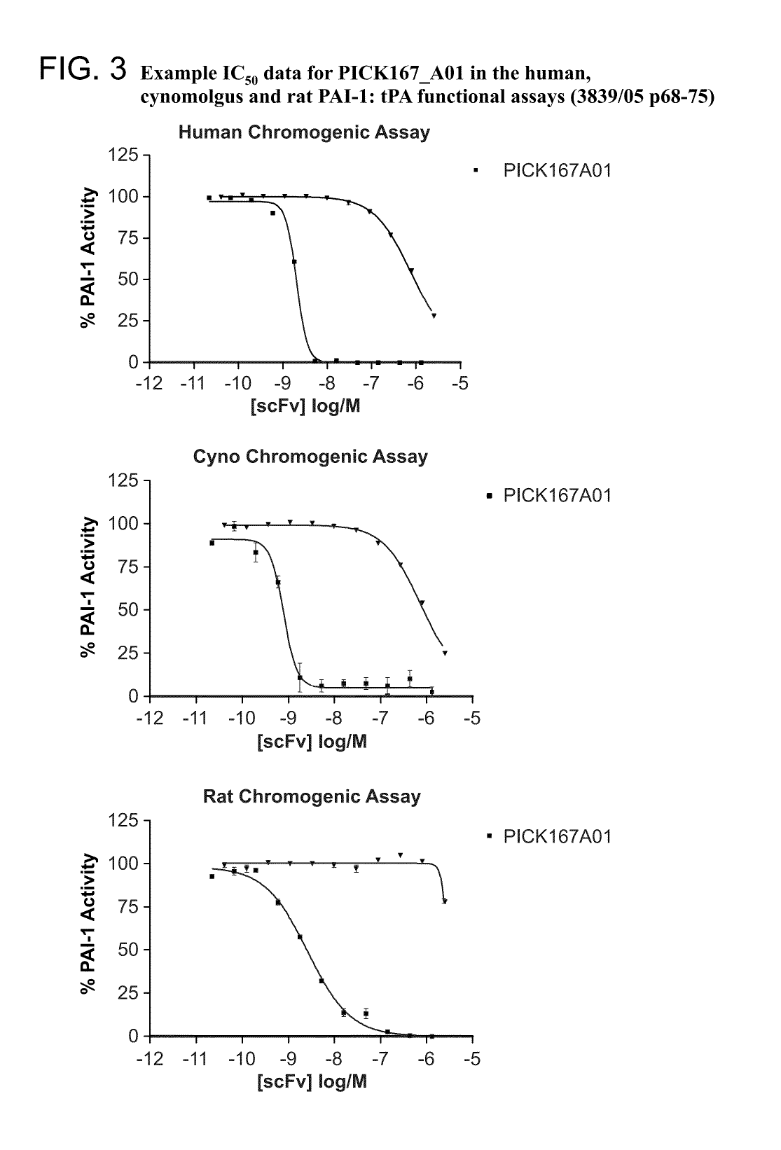

This example details the characterization of one antibody, PICK167_A01. PICK167_A01 scFv inhibited the action of human and cynomolgus PAI-1 (both 5.0 nM) on human tPA (2.14 nM) with IC₅₀ values of 3.20 nM and 1.02 nM, respectively. PICK167_A01 scFv inhibited the action of rat PAI-1 (2.0 nM) on rat tPA (1.34 nM) with IC₅₀ 2.98 nM. Example IC₅₀ data for PICK167_A01 scFv in each of these assays is shown in FIG. 3.

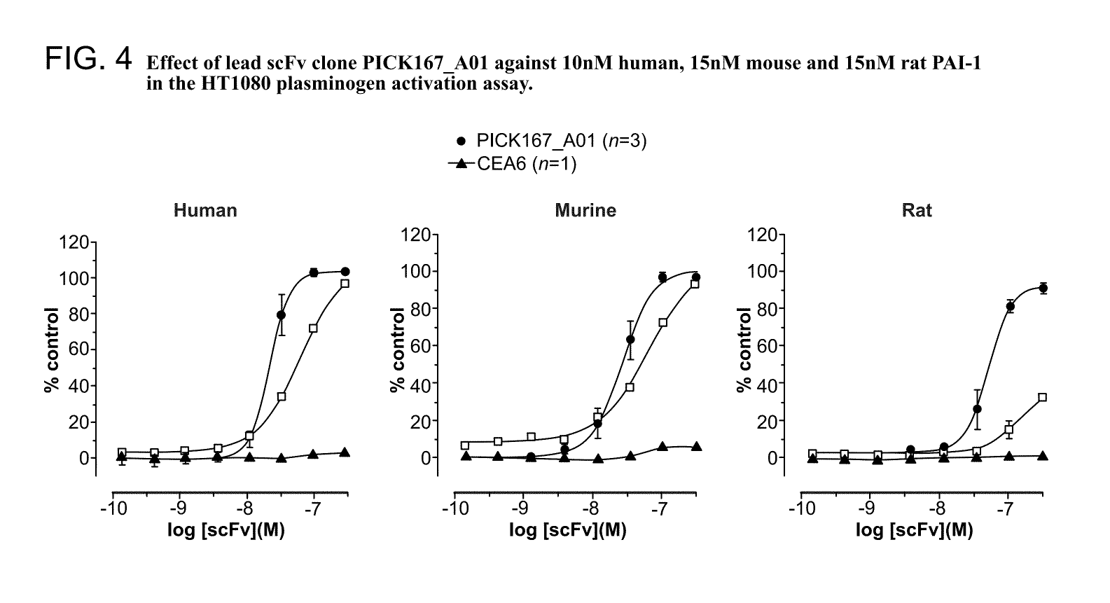

Additionally, PICK167_A01 scFv neutralised 10 nM human, 15 nM mouse and 15 nM rat PAI-1 in a cell-based HT1080 plasminogen activation assay (see FIG. 4), with the pIC₅₀ₛ of 7.65±0.04 (22 nM), 7.60±0.048 (25 nM) and 7.31±0.04 (49 nM) respectively.

PICK167_A01 Sequence and Conversion to IgG₁

```
                10 20 30 40 50
CCGGCCATGGCCCAGGTACAGCTGCAGCAGTCAGGGGCTGAAGTGAAGAA
GGCCGGTACCGGGTCCATGTCGACGTCGTCAGTCCCCGACTTCACTTCTT
  P A M A Q V Q L Q Q S G A E V K K>
_____TRANSLATION OF PICK167_A01.DNA.MCV [A]______>
                60 70 80 90 100
GCCTGGCTCTTCGGTGAAGGTCTCCTGCAAGGCTTCTGGAGGCACCTTCA
CGGACCGAGAAGCCACTTCCAGAGGACGTTCCGAAGACCTCCGTGGAAGT
    P G S S V K V S C K A S G G T F>
_____TRANSLATION OF PICK167_A01.DNA.MCV [A]______>
              110 120 130 140 150
GCAGCTACGCTATCAGCTGGGTGCGACAGGCCCCTGGACAAAGGCTTGAG
CGTCGATGCGATAGTCGACCCACGCTGTCCGGGGACCTGTTTCCGAACTC
S S Y A I S W V R Q A P G Q R L E>
_____TRANSLATION OF PICK167_A01.DNA.MCV [A]______>
              160 170 180 190 200
TGGATGGGAGGGATCATCCCTACTTTTGGTACAGCAAACTACGCTCAGAA
ACCTACCCTCCCTAGTAGGGATGAAAACCATGTCGTTTGATGCGAGTCTT
  W M G G I I P T F G T A N Y A Q K>
_____TRANSLATION OF PICK167_A01.DNA.MCV [A]______>
              210 220 230 240 250
GTTCCAGGGCAGAGTCACGATTACCGCGGACGAATCCACGAGCACAGCCT
CAAGGTCCCGTCTCAGTGCTAATGGCGCCTGCTTAGGTGCTCGTGTCGGA
    F Q G R V T I T A D E S T S T A>
_____TRANSLATION OF PICK167_A01.DNA.MCV [A]______>
              260 270 280 290 300
ACATGGAGCTGAGCGGCCTGAGGTCTGAGGACACGGCCGTCTATTACTGT
TGTACCTCGACTCGCCGGACTCCAGACTCCTGTGCCGGCAGATAATGACA
Y M E L S G L R S E D T A V Y Y C>
_____TRANSLATION OF PICK167_A01.DNA.MCV [A]______>
              310 320 330 340 350
GCGAGAGAGAGGAGGCAGTGGCTGGAAGGACACTTTGACTACTGGGGCCG
CGCTCTCTCTCCTCCGTCACCGACCTTCCTGTGAAACTGATGACCCCGGC
  A R E R R Q W L E G H F D Y W G R>
_____TRANSLATION OF PICK167_A01.DNA.MCV [A]______>
              360 370 380 390 400
GGGCACCCCGGTCACCGTCTCGAGTGGTGGAGGCGGCTCTGGCGGAGGTG
CCCGTGGGGCCAGTGGCAGAGCTCACCACCTCCGCCGAGACCGCCTCCAC
    G T P V T V S S G G G G S G G G>
_____TRANSLATION OF PICK167_A01.DNA.MCV [A]______>
              410 420 430 440 450
GCAGCGGCGGTGGCGGATCGGACATCCAGATGACCCAGTCCCCATCCTTC
CGTCGCCGCCACCGCCTAGCCTGTAGGTCTACTGGGTCAGGGGTAGGAAG
G S G G G G S D I Q M T Q S P S F>
_____TRANSLATION OF PICK167_A01.DNA.MCV [A]______>
              460 470 480 490 500
CTGTCTGCATCTATTGGAGACAGAGTCACCATCACCTGCCGGGCCAGTGA
GACAGACGTAGATAACCTCTGTCTCAGTGGTAGTGGACGGCCCGGTCACT
  L S A S I G D R V T I T C R A S E>
_____TRANSLATION OF PICK167_A01.DNA.MCV [A]______>
              510 520 530 540 550
GGGTATTTATCACAACTTGGCCTGGTATCAGCAGAAGCCAGGGAAAGCCC
CCCATAAATAGTGTTGAACCGGACCATAGTCGTCTTCGGTCCCTTTCGGG
    G I Y H N L A W Y Q Q K P G K A>
_____TRANSLATION OF PICK167_A01.DNA.MCV [A]______>
              560 570 580 590 600
CAAAACTCCTGATCTATAAGGCCTCTAGTTTAGCCAGTGGGGCCCCATCA
GTTTTGAGGACTAGATATTCCGGAGATCAAATCGGTCACCCCGGGGTAGT
P K L L I Y K A S S L A S G A P S>
_____TRANSLATION OF PICK167_A01.DNA.MCV [A]______>
              610 620 630 640 650
AGGTTCAGCGGCAGTAGATCTGGGACAGATTTCACTCTCACCATCAGCAG
TCCAAGTCGCCGTCATCTAGACCCTGTCTAAAGTGAGAGTGGTAGTCGTC
  R F S G S R S G T D F T L T I S S>
_____TRANSLATION OF PICK167_A01.DNA.MCV [A]______>
              660 670 680 690 700
CCTGCAGCCTGATGATTTTGCGACTTATTACTGCCAACAATATAGTAATT
GGACGTCGGACTACTAAAACGCTGAATAATGACGGTTGTTATATCATTAA
    L Q P D D F A T Y Y C Q Q Y S N>
_____TRANSLATION OF PICK167_A01.DNA.MCV [A]______>
              710 720 730 740
ATCCACTTACTTTCGGCGGAGGGACCAAGCTGGAGATCAAACGTGCG
TAGGTGAATGAAAGCCGCCTCCCTGGTTCGACCTCTAGTTTGCACGC
Y P L T F G G G T K L E I K R A>
____TRANSLATION OF PICK167_A01.DNA.MCV [A]____>
```

PICK167_A01 scFv was converted to IgG₁ by sub-cloning the V_{H} and V_{L} domains into vectors expressing whole human antibody heavy and light chains respectively. The variable heavy chain was cloned into the pEU15.1 vector containing the human heavy chain constant domains and regulatory elements to express whole IgG₁ heavy chain in mammalian cells. Similarly, the variable light chain domain was cloned into the pEU3.4 vector for the expression of the human light chain (kappa) constant domains and regulatory elements to express whole IgG light chain in mammalian cells (Persic et al 1997). To obtain the PICK167_A01 antibody as IgG₁, the heavy and light chain IgG expressing vectors were transiently transfected into EBNA-HEK293 mammalian cells and secreted into the medium. Harvests were pooled and filtered prior to purification, and PICK167_A01 antibody was purified using Protein A chromatography.

### I. Specificity of PICK167_A01 IgG₁

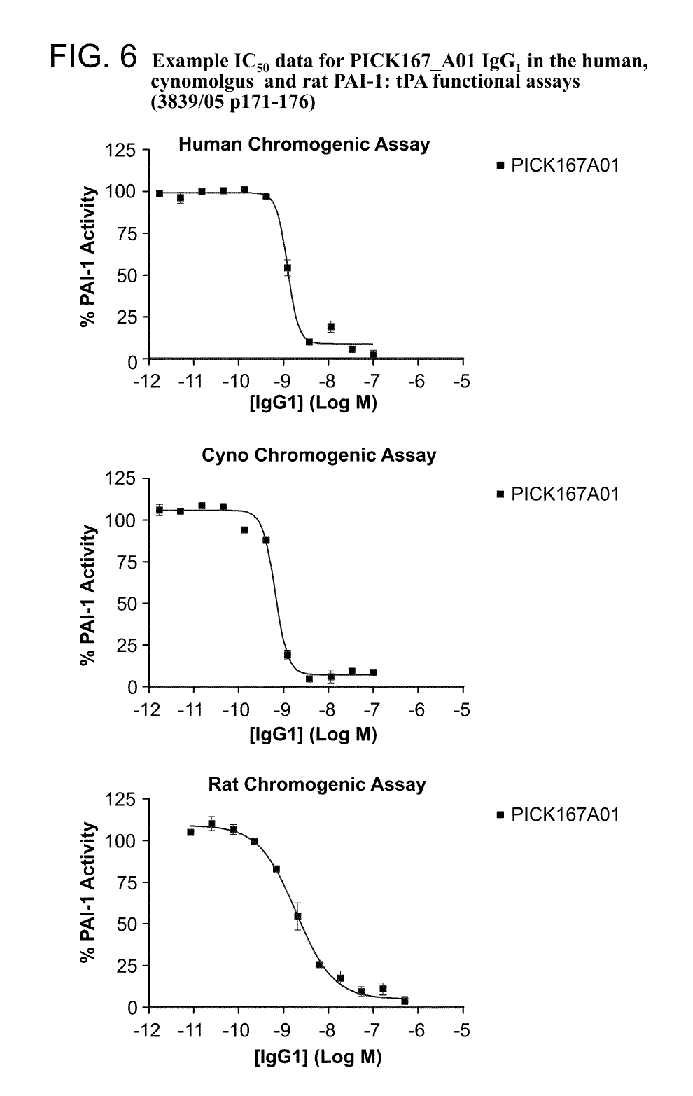

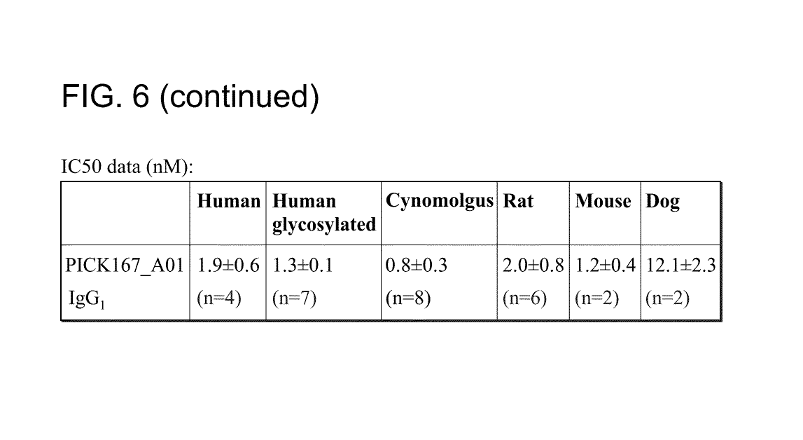

PICK167_A01 IgG₁ cross-reactivity to rat, murine, rabbit and latent PAI-1, and specificity for PAI-1 above a panel of structurally related serpins, was assessed. Briefly, PICK167_A01 IgG₁ was coated onto wells of a Maxisorp plate. Binding of biotinylated PAI-1 in the presence of a titration of test antigen was detected by the addition of Streptavidin-Eu³⁺ using the DELFIA system (Perkin Elmer). Data were transferred to Prism where a one-site binding analysis was carried out. Results are shown in FIGS. 5 (a) and (b). Functional cross reactivity to rat PAI-1 is also shown in FIG. 6.

PICK167_A01 demonstrates a 10-fold loss of binding affinity for latent PAI-1 compared to wild-type ‘active’ PAI-1 (labelled as ‘human’ throughout the figures below) (FIG. 5 (a)). PICK167_A01 does not bind to a panel of other human serpins when tested by this method (FIG. 5 (b)). PICK167_A01 did not inhibit binding of PAI-1 to vitronectin in a biochemical assay in either scFv or IgG₁ formats.

### J. In vitro potency of PICK167_A01 IgG₁

PICK167_A01 IgG₁ was then further characterised in secondary functional assays in which the activity of tPA in the presence of both PAI-1 and PICK167_A01 IgG₁ was measured. Human, cynomolgus and rat versions of this generic assay format were used to confirm human potency and functional cross-reactivity. PICK167_A01 IgG₁ inhibited the action of human and cynomolgus PAI-1 (both 5.0 nM) on human tPA (2.14 nM) with IC₅₀ values of 1.34+/−0.15 nM and 0.80+/−0.31 nM, respectively. PICK167_A01 IgG₁ inhibited the action of rat PAI-1 (2.0 nM) on rat tPA (1.34 nM) with IC₅₀ 1.97+/−0.77 nM. Example IC₅₀ data for PICK167_A01 IgG₁ in each of these assays is shown in FIG. 6.

### K. Reversion to Germline and Conversion to IgG1

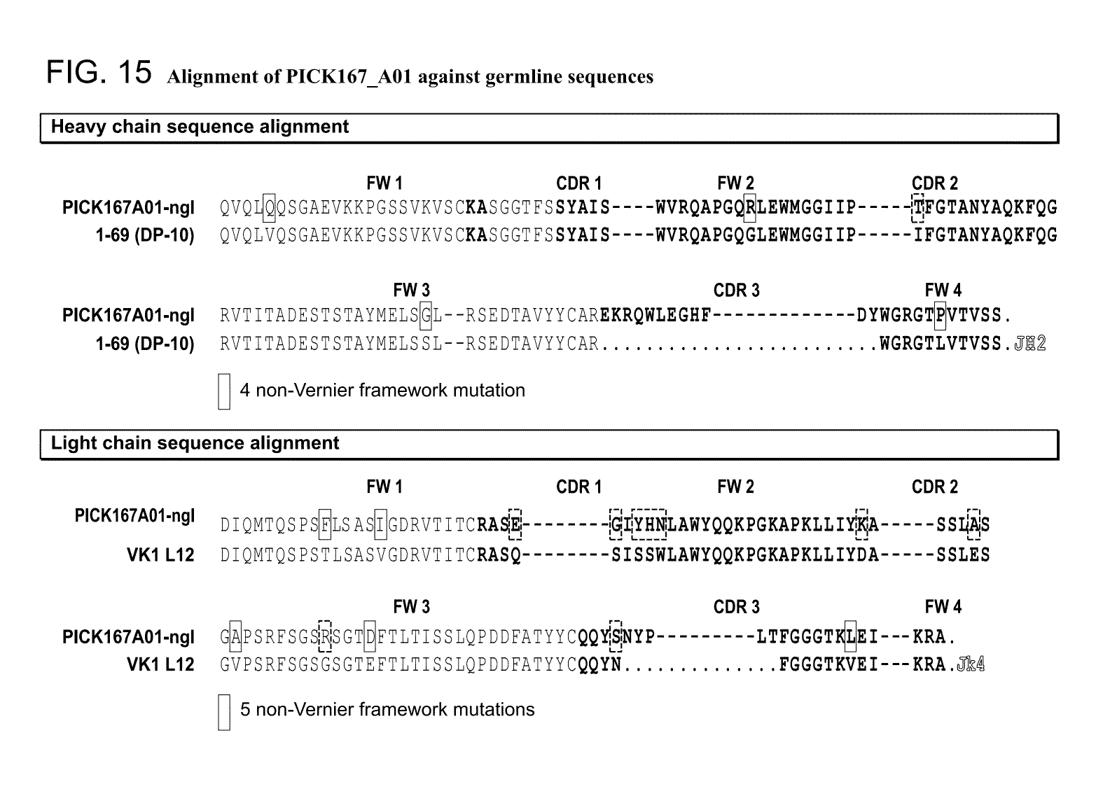

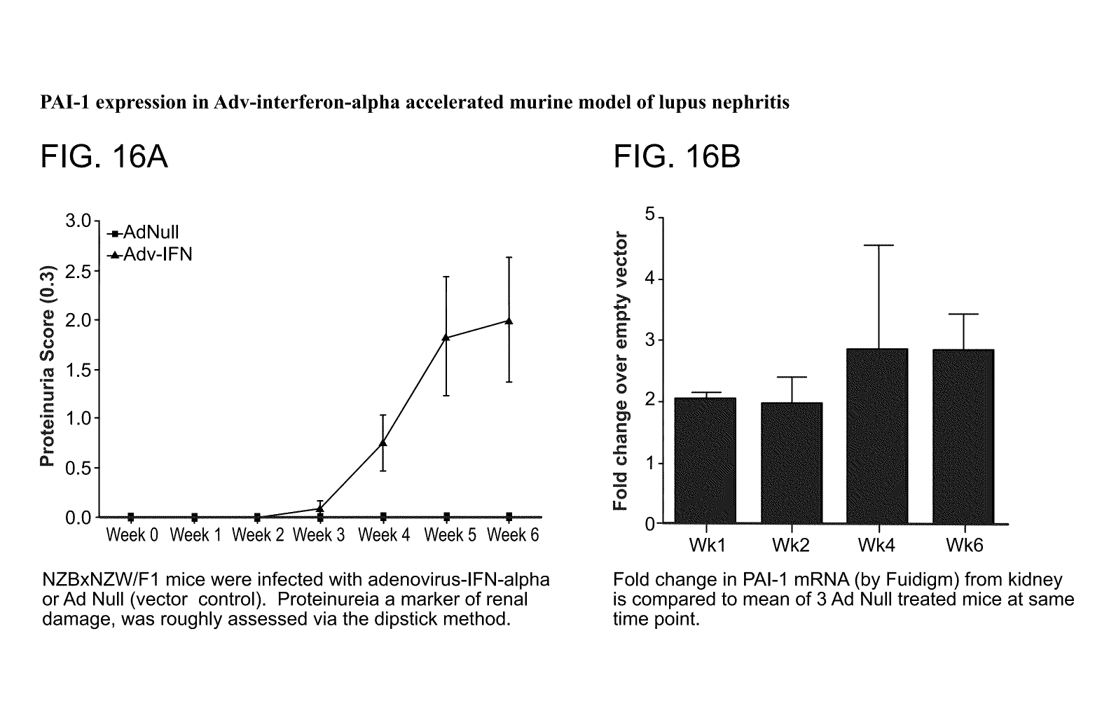

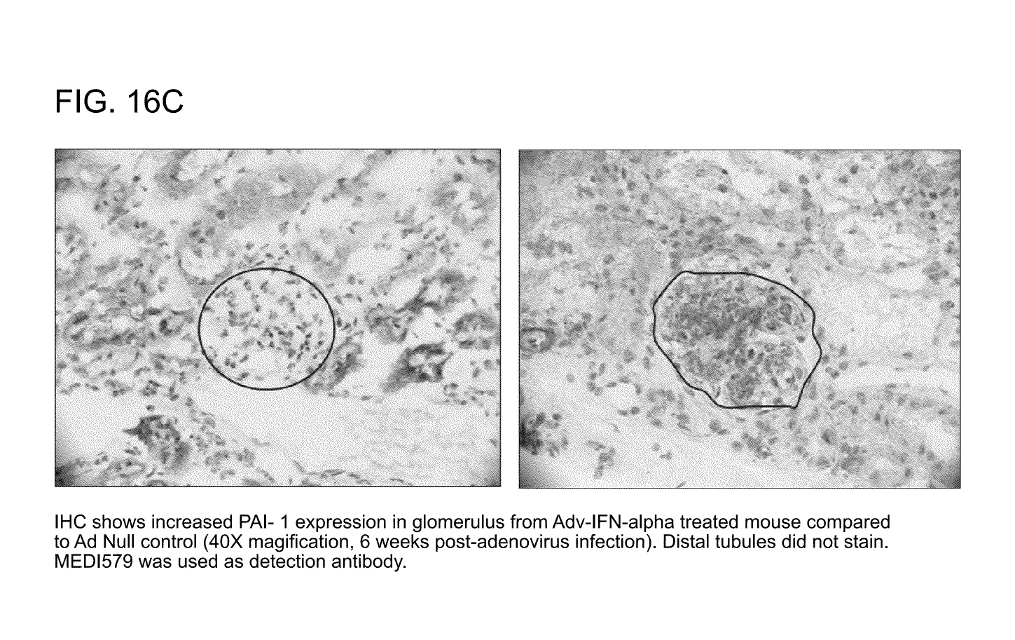

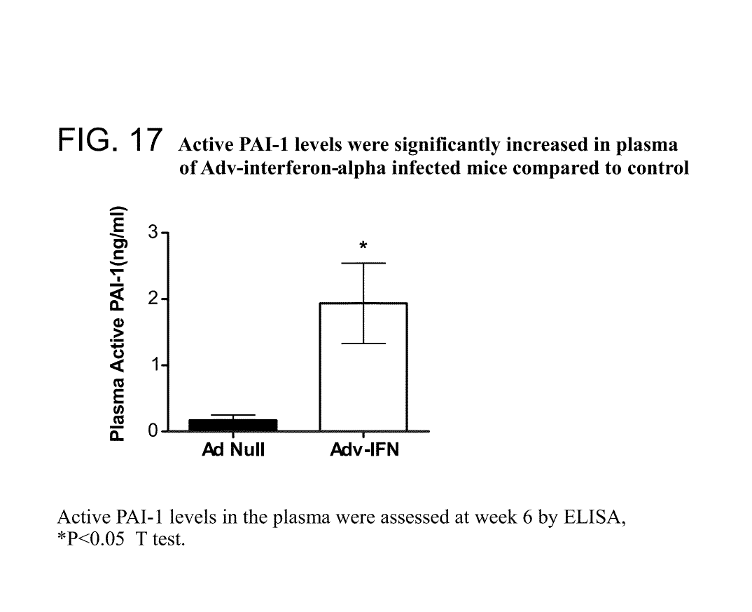

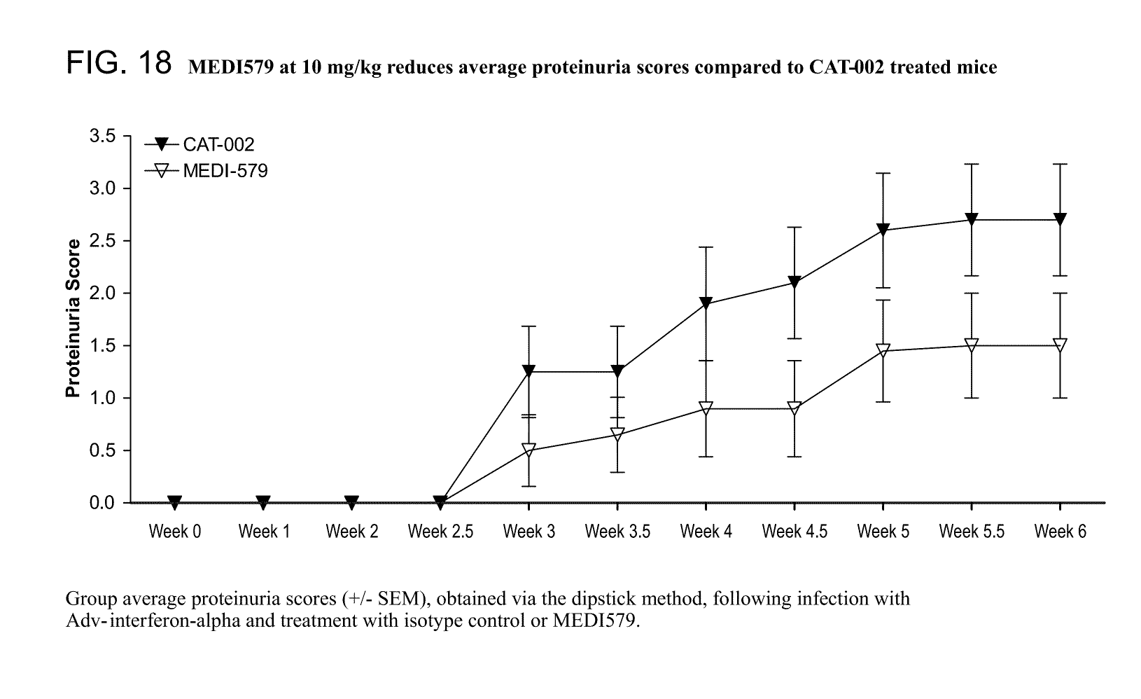

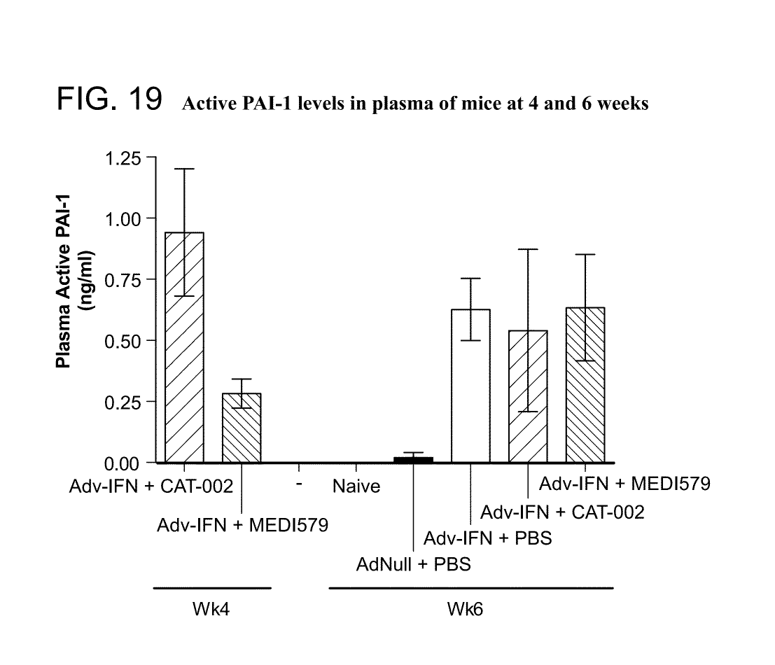

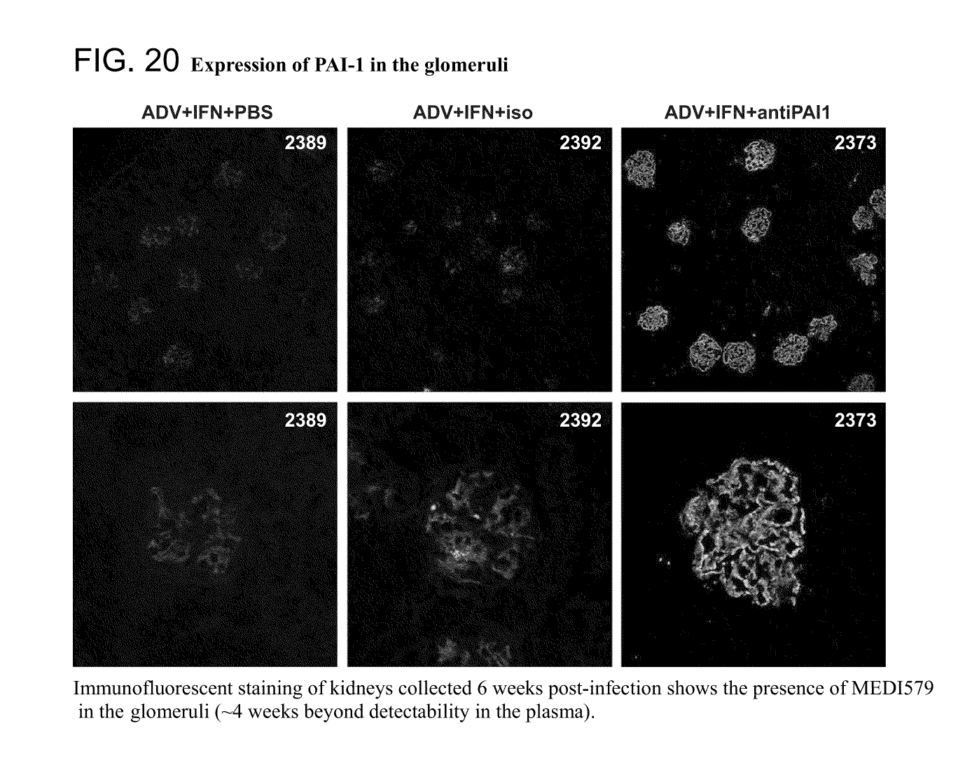

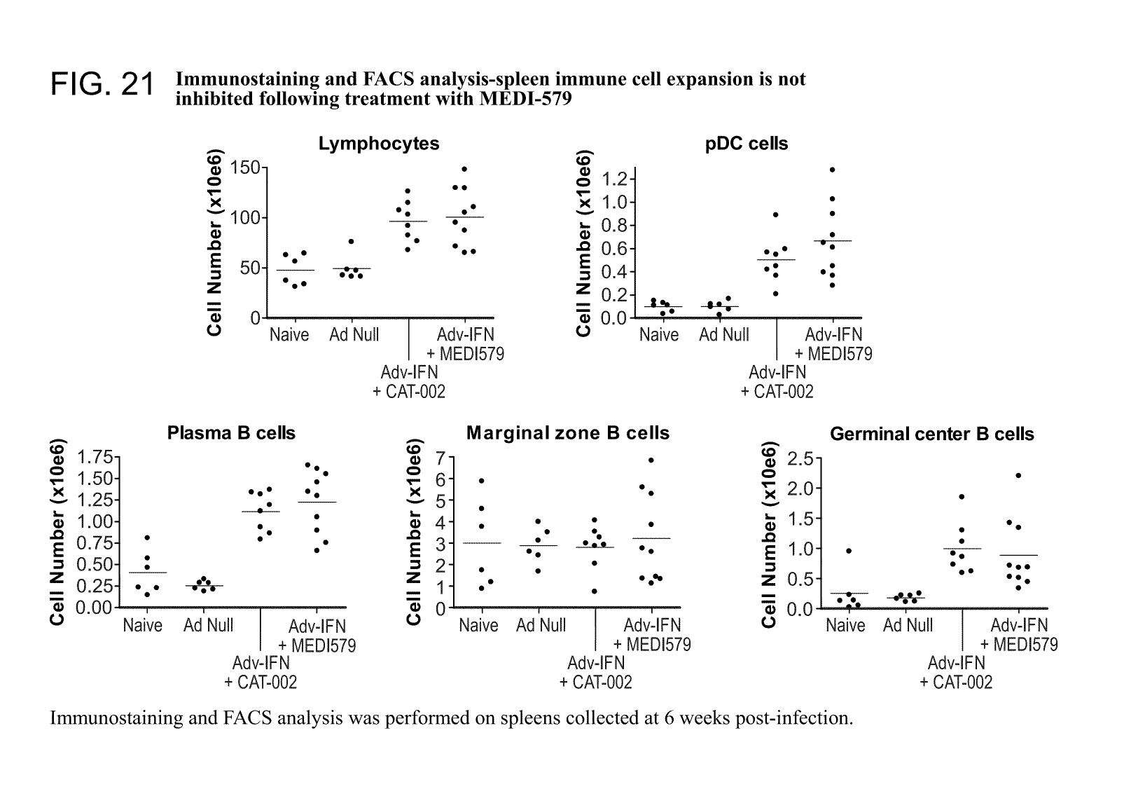

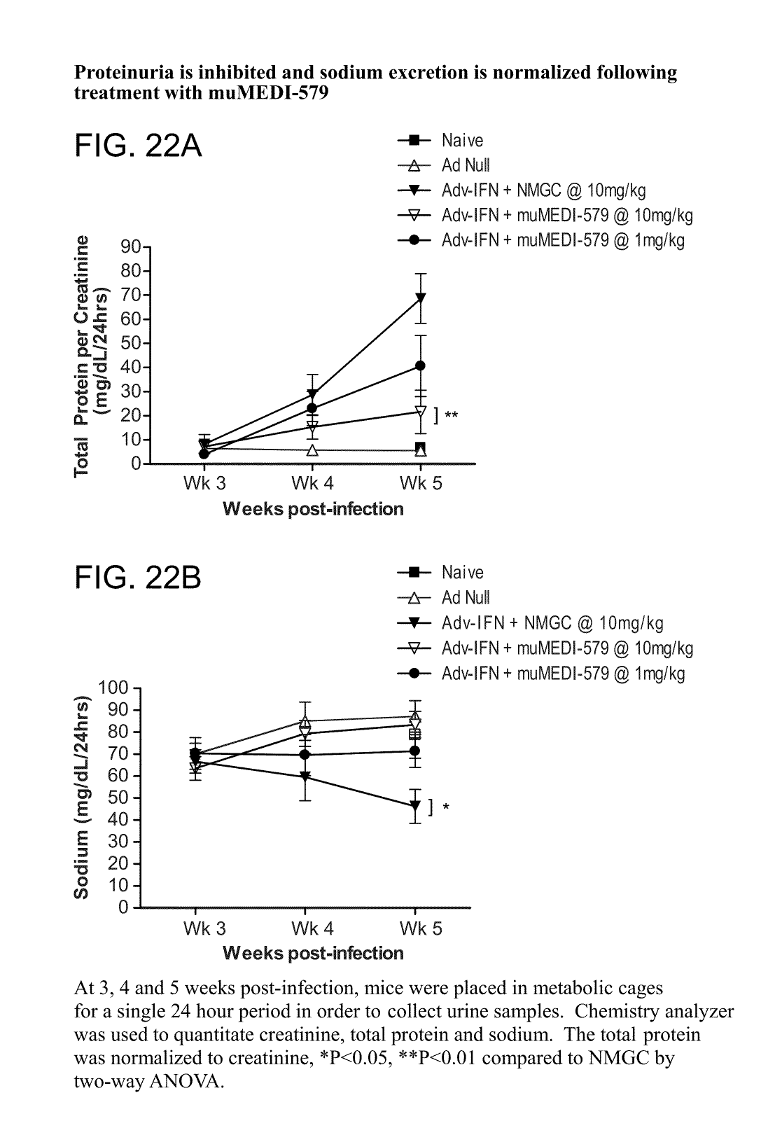

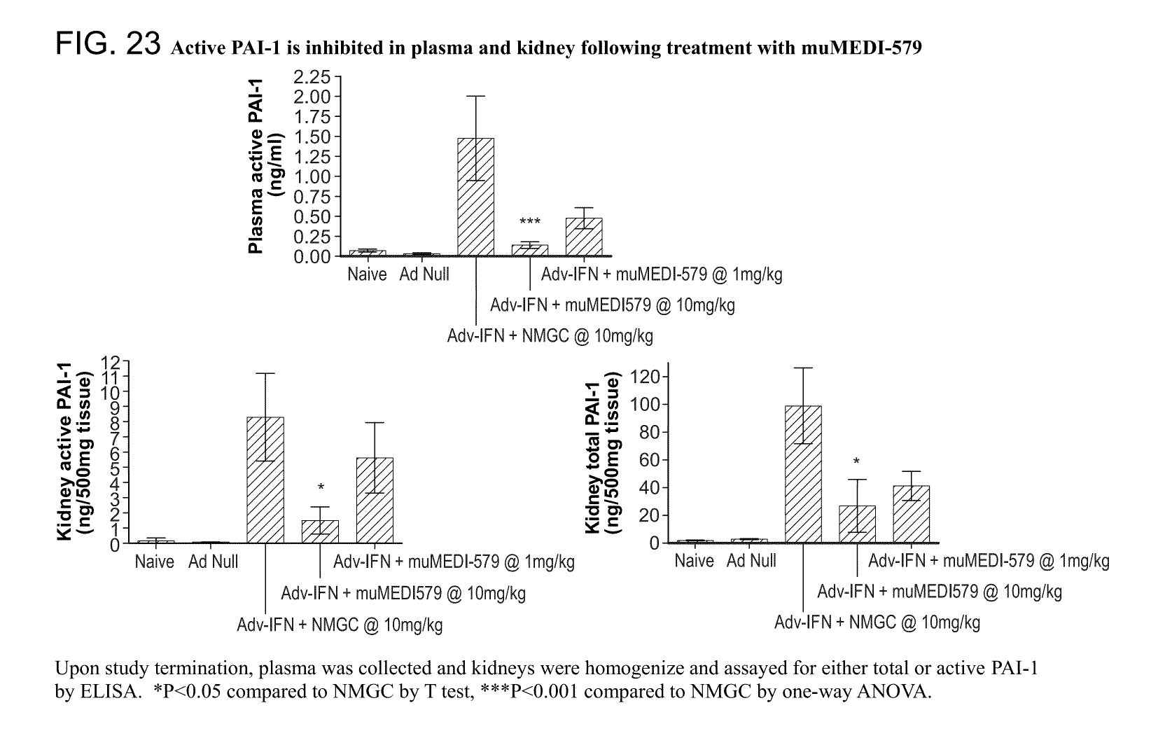

The amino acid sequences of the VH and VL domains of PICK167_A01 were compared to the known human germline sequences in the VBASE database (Tomlinson 1997), and the closest germline identified by sequence similarity. The closest human germline sequences for the heavy and light chains were found to be VH 1-69 (DP10) and VK L12a respectively. An alignment of PICK167_A01 to these, and closest matching J regions, is shown in FIG. 15 with differences between PICK167_A01 and germline sequences highlighted.

Vernier residues (Foote and Winter, 1992) that remained unmutated are indicated in grey.

Without considering the Vernier residues, there were four changes made in the light chain and five made in the heavy chain. Mutagenesis was by the method of Kunkel (Sidhu and Weiss, 1990), adapted for mutagenesis in the IgG vectors containing the separate VH and VL domains of PICK167_A01 as template. This resulted in the creation of the fully-germlined variant, PICK167_A01-fgl (CAT-1001) as an IgG1 only. The sequences of PICK167_A01-fgl DNA and deduced amino acid sequence are shown below.

PICK167_A01-fgl VH

```
                10 20 30 40 50
CAGGTACAGCTGGTGCAGTCAGGGGCTGAGGTGAAGAAGCCTGGCTCTTC
GTCCATGTCGACCACGTCAGTCCCCGACTCCACTTCTTCGGACCGAGAAG
  Q V Q L V Q S G A E V K K P G S S>
__TRANSLATION OF PICK167_A01VH.FGL.DNA.MCV [A]___>
                60 70 80 90 100
GGTGAAGGTCTCCTGCAAGGCTTCTGGAGGCACCTTCAGCAGCTACGCTA
CCACTTCCAGAGGACGTTCCGAAGACCTCCGTGGAAGTCGTCGATGCGAT
    V K V S C K A S G G T F S S Y A>
__TRANSLATION OF PICK167_A01VH.FGL.DNA.MCV [A]___>
              110 120 130 140 150
TCAGCTGGGTGCGACAGGCCCCTGGACAAGGGCTTGAGTGGATGGGAGGG
AGTCGACCCACGCTGTCCGGGGACCTGTTCCCGAACTCACCTACCCTCCC
I S W V R Q A P G Q G L E W M G G>
__TRANSLATION OF PICK167_A01VH.FGL.DNA.MCV [A]___>
              160 170 180 190 200
ATCATCCCTACTTTTGGTACAGCAAACTACGCTCAGAAGTTCCAGGGCAG
TAGTAGGGATGAAAACCATGTCGTTTGATGCGAGTCTTCAAGGTCCCGTC
  I I P T F G T A N Y A Q K F Q G R>
__TRANSLATION OF PICK167_A01VH.FGL.DNA.MCV [A]___>
              210 220 230 240 250
AGTCACGATTACCGCGGACGAATCCACGAGCACAGCCTACATGGAGCTGA
TCAGTGCTAATGGCGCCTGCTTAGGTGCTCGTGTCGGATGTACCTCGACT
    V T I T A D E S T S T A Y M E L>
__TRANSLATION OF PICK167_A01VH.FGL.DNA.MCV [A]___>
              260 270 280 290 300
GCAGCCTGAGGTCTGAGGACACGGCCGTCTATTACTGTGCGAGAGAGAGG
CGTCGGACTCCAGACTCCTGTGCCGGCAGATAATGACACGCTCTCTCTCC
S S L R S E D T A V Y Y C A R E R>
__TRANSLATION OF PICK167_A01VH.FGL.DNA.MCV [A]___>
              310 320 330 340 350
AGGCAGTGGCTGGAAGGACACTTTGACTACTGGGGCCGGGGCACCCTGGT
TCCGTCACCGACCTTCCTGTGAAACTGATGACCCCGGCCCCGTGGGACCA
  R Q W L E G H F D Y W G R G T L V>
__TRANSLATION OF PICK167_A01VH.FGL.DNA.MCV [A]___>
              360
CACCGTCTCCTCA
GTGGCAGAGGAGT
    T V S S>
____________>
```

4. PICK167_A01-fgl VL

```
                10 20 30 40 50
GACATCCAGATGACCCAGTCCCCATCCACCCTGTCTGCATCTGTTGGAGA
CTGTAGGTCTACTGGGTCAGGGGTAGGTGGGACAGACGTAGACAACCTCT
  D I Q M T Q S P S T L S A S V G D>
__TRANSLATION OF PICK167_A01VL.FGL.DNA.MCV [A]___>
                60 70 80 90 100
CAGAGTCACCATCACCTGCCGGGCCAGTGAGGGTATTTATCACAACTTGG
GTCTCAGTGGTAGTGGACGGCCCGGTCACTCCCATAAATAGTGTTGAACC
    R V T I T C R A S E G I Y H N L>
__TRANSLATION OF PICK167_A01VL.FGL.DNA.MCV [A]___>
              110 120 130 140 150
CCTGGTATCAGCAGAAGCCAGGGAAAGCCCCAAAACTCCTGATCTATAAG
GGACCATAGTCGTCTTCGGTCCCTTTCGGGGTTTTGAGGACTAGATATTC
A W Y Q Q K P G K A P K L L I Y K>
__TRANSLATION OF PICK167_A01VL.FGL.DNA.MCV [A]___>
              160 170 180 190 200
GCCTCTAGTTTAGCCAGTGGGGTCCCATCAAGGTTCAGCGGCAGTAGATC
CGGAGATCAAATCGGTCACCCCAGGGTAGTTCCAAGTCGCCGTCATCTAG
  A S S L A S G V P S R F S G S R S>
__TRANSLATION OF PICK167_A01VL.FGL.DNA.MCV [A]___>
              210 220 230 240 250
TGGGACAGAGTTCACTCTCACCATCAGCAGCCTGCAGCCTGATGATTTTG
ACCCTGTCTCAAGTGAGAGTGGTAGTCGTCGGACGTCGGACTACTAAAAC
    G T E F T L T I S S L Q P D D F>
__TRANSLATION OF PICK167_A01VL.FGL.DNA.MCV [A]___>
              260 270 280 290 300
CGACTTATTACTGCCAACAATATAGTAATTATCCACTTACTTTCGGCGGA
GCTGAATAATGACGGTTGTTATATCATTAATAGGTGAATGAAAGCCGCCT
A T Y Y C Q Q Y S N Y P L T F G G>
__TRANSLATION OF PICK167_A01VL.FGL.DNA.MCV [A]___>
              310 320
GGGACCAAGGTGGAGATCAAACGT
CCCTGGTTCCACCTCTAGTTTGCA
  G T K V E I K R>
___TRANSLATION OF PI___>
```

IgG₁ formatting occurred in the lead optimisation phase during the characterisation of project leads. As described above, the fully-germlined lead, PICK167_A01-fgl, was generated using these IgG constructs as templates. Accordingly, CAT-1001 has been converted to IgG₁ during this process.

### L. Specificity of CAT-1001 (PICK67A01-fgl)

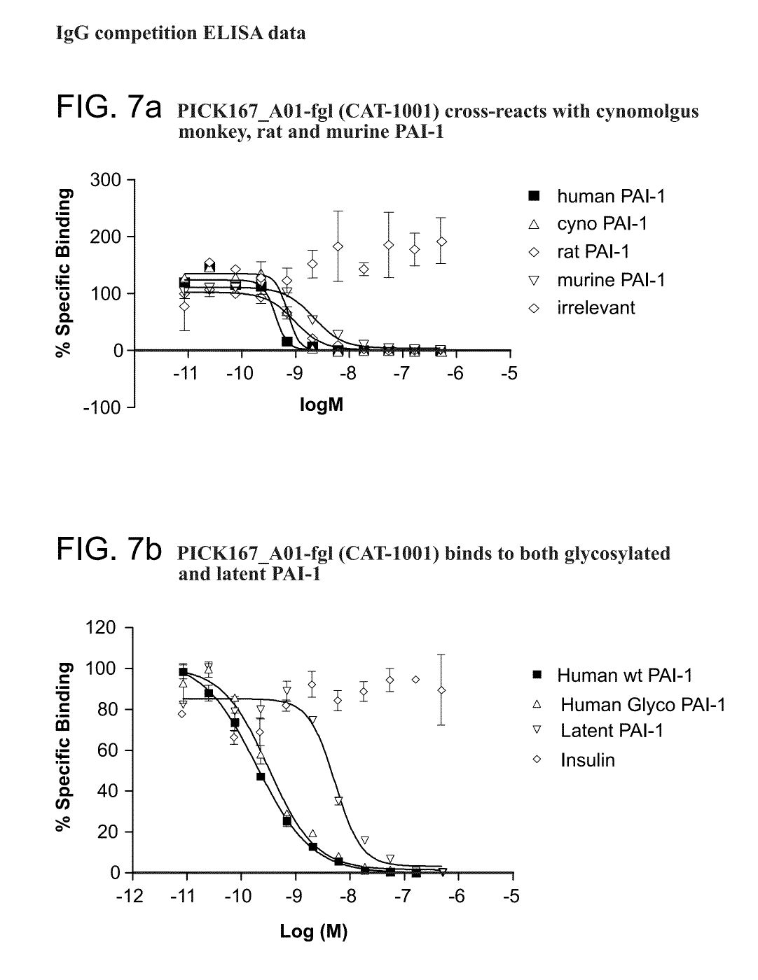

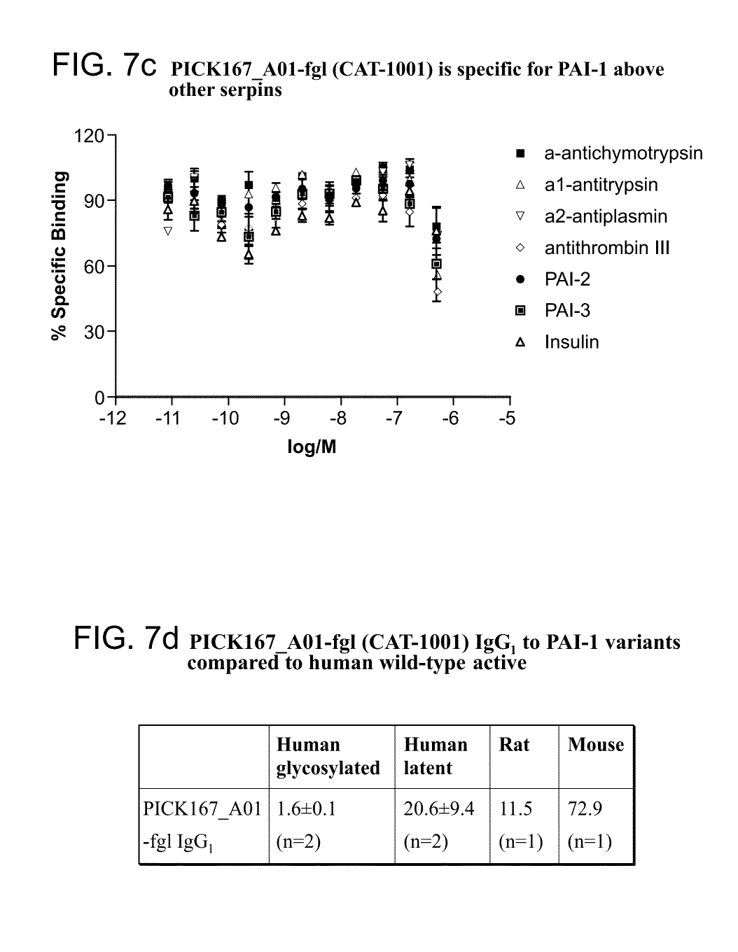

CAT-1001 (PICK67A01-fgl) cross-reactivity to rat, murine, cynomolgus monkey, latent and glycosylated human PAI-1, and specificity for human PAI-1 above a panel of structurally related serpins, was assessed. Briefly, PICK167_A01-fgl IgG₁ was coated onto wells of a Maxisorp plate. Binding of biotinylated PAI-1 in the presence of a titration of test antigen was detected by the addition of Streptavidin-Eu³⁺ using the DELFIA system (Perkin Elmer). Data were transferred to Prism where a one-site binding analysis was carried out. Results are shown in FIG. 7. PICK167_A01-fgl demonstrates a 20-fold loss of binding affinity for latent PAI-1 compared to wild-type ‘active’ PAI-1 (labelled as ‘human’ throughout the figures), while there is no significant difference between binding of PICK167_A01-fgl to glycosylated or recombinant human PAI-1 (FIG. 7 (b)). PICK167_A01-fgl does not bind to a panel of other human serpins when tested by this method (FIG. 7 (c)).

### M. In Vitro Potency and Kinetics of CAT-1001 (PICK67A01-Fgl)

CAT-1001 (PICK167_A01-fgl IgG₁) was then further characterised in secondary functional assays as described previously in which the activity of tPA in the presence of both PAI-1 and CAT-1001 (PICK167_A01-fgl IgG₁) was measured. Human, cynomolgus and rat versions of this generic assay format were again used to confirm both human potency and also functional cross-reactivity. CAT-1001 (PICK167_A01-fgl IgG₁) inhibited the action of human and cynomolgus PAI-1 (both 5.0 nM) on human tPA (2.14 nM) with IC₅₀ values of 1.65 nM and 0.77 nM, respectively. CAT-1001 (PICK167_A0′-fgl IgG₁) inhibited the action of rat PAI-1 (2.0 nM) on rat tPA (1.34 nM) with IC₅₀ 3.07 nM. Example IC₅₀ data for CAT-1001 (PICK167_A01-fgl IgG₁) in each of these assays is shown in FIG. 8.

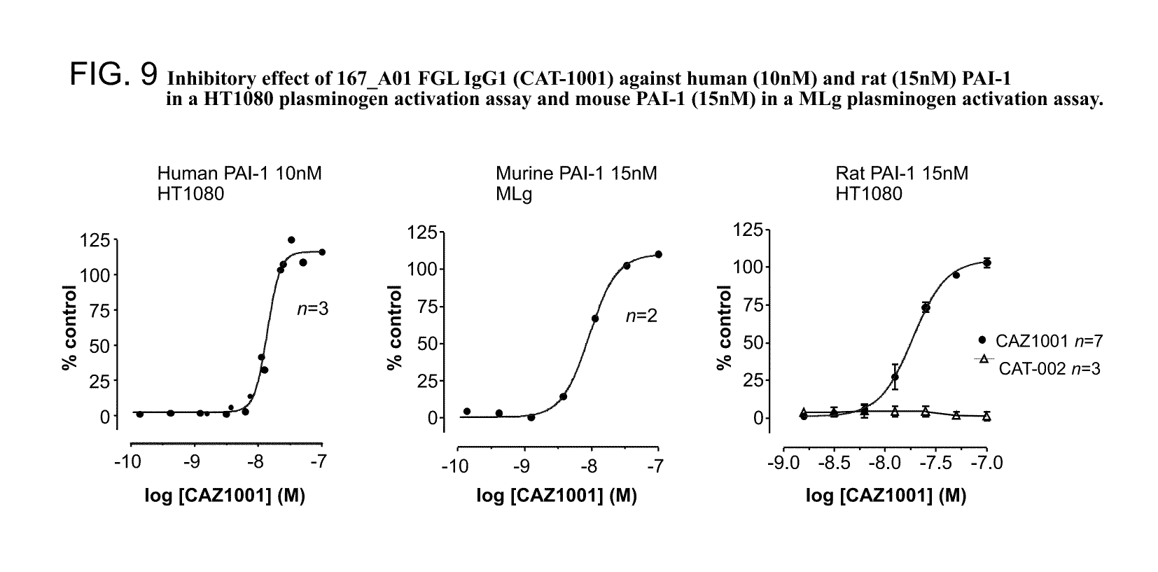

PICK167_A01-fgl IgG₁ (CAT-1001) inhibited 10 nM human and 15 nM rat PAI-1 in a cell-based HT1080 plasminogen activation assay with pIC₅₀ values of 7.87±0.04 (13.2 nM) and 7.64±0.03 (22.8 nM) respectively. PICK167_A01-fgl IgG₁ also inhibited 15 nM mouse PAI-1 in a mouse MLg cell plasminogen activation assay with a pIC₅₀ of 7.97±0.18 (10.6 nM) (FIG. 9).

The affinity for CAT-1001 and human PAI-1 was measured by BIAcore analysis. In brief, CAT-1001 was amino-linked to a CM3 BIAcore chip and serial dilutions (200-0.78 nM) of human wild-type PAI-1 was used as the analyte. The data were fitted to a 1:1 Langmuir dissociation model. The affinity of CAT-1001 for human PAI-1 was determined as 16 pM with average Kₒₙ and K_{off} values of 2.27×10⁶ M⁻¹s⁻¹ and 3.67×10⁻⁵ s⁻¹ respectively.

### Example 5

### Cloning of Cynomolgus PAI-1 and Dog PAI-1 Sequences

Cynomolgus PAI-1 cDNA sequence
ATGCAGATGTCTCCAGCCCTCGCCTGCCTAGTCCTGGGCCTGGCCTTC
GTCTTTGGTGAAGGGTCCACCGTGCACCATCCCCCATCCTACGTGGCC
CACCTGGCCTCAGACTTCGGGGTTAGGGTGTTTCAGCAGGTGGCGCAG
GCCTCCAAAGACCGCAACGTGGTTTTCTCACCCTATGGGGTGGCCTCG
GTGTTGGCCATGCTCCAGCTGACAACGGGAGGAGAAACCCGGCAGCAG
ATCCAAGCGGCCATGGGATTCAAGATTGATGACAAGGGCATGGCTCCC
GCCCTCCGGCATCTGTACAAGGAGCTCTTGGGGCCGTGGAACAAGGAT
GAGATCAGCACCACAGACGCGATCTTTGTCCAGCGGGATCTGAAGCTG
GTCCAGGGCTTCATGCCCCACTTCTTCAGGCTGTTCCGGAGTACGGTC
AAGCAGGTGGACTTTTCAGAGGCAGAGAGAGCCAGATTCATCATCAAC
GACTGGGTGAAGACACACACAAAAGGGATGATCAGTGACTTGCTTGGG
AAAGGAGCCGTGGACCAGCTGACACGGCTGGTGCTGGTGAACGCCCTC
TACTTCAACGGCCACTGGAAGACTCCCTTCCCCGACTCCAGCACCCAC
CGCCGCCTCTTCCACAAATCAGATGGCAGCACTGTCTCTGTGCCCATG
ATGGCTCAGACCAACAAGTTCAACTATACCGAGTTCACCACGCCCGAT
GGCCATTACTACGACATCCTGGAACTGCCCTACCACGGAAACACCCTC
AGCATGTTCATTGCTGCCCCTTATGAAAAGCAGGTGCCTCTCTCTGCC
CTCACCAACATTCTGAGTGCCCAGCTCATCAGCCACTGGAAAGGCAAC
ATGACCAGGCTGCCCCGCCTCCTGGTTCTGCCCAAGTTCTCCCTGGAG
ACTGAAGTTGACCTCAGGAAGCCCCTAGAGAACCTGGGAATGACCGAC
ATGTTCAGACAATTTCAGGCGGACTTCACGAGTCTTTCAAACCAAGAG
CCTCTCCACGTTGCGCAAGCGCTTCAGAAAGTGAAGATCGAGGTGAAC
GAGAGTGGCACGGTGGCCTCCTCATCCACAGCTGTCATAGTCTCAGCC
CGGATGGCGCCTGAGGAGATCATCATGGACAGACCCTTCCTCTTTGTG
GTCCGGCACAACCCCACAGGAACAGTCCTTTTCATGGGCCAAGTGATG
GAACCTTGA
N. Cynomolgus PAI-1 derived amino acid sequence
(leader sequence underline)
MQMSPALACLVLGLAFVFGEGSTVHHPPSYVAHLASDFGVRVFQQVAQ
ASKDRNVVFSPYGVASVLAMLQLTTGGETRQQIQAAMGFKIDDKGMAP
ALRHLYKELLGPWNKDEISTTDAIFVQRDLKLVQGFMPHFFRLFRSTV
KQVDFSEAERARFIINDWVKTHTKGMISDLLGKGAVDQLTRLVLVNAL
YFNGHWKTPFPDSSTHRRLFHKSDGSTVSVPMMAQTNKFNYTEFTTPD
GHYYDILELPYHGNTLSMFIAAPYEKQVPLSALTNILSAQLISHWKGN
MTRLPRLLVLPKFSLETEVDLRKPLENLGMTDMFRQFQADFTSLSNQE
PLHVAQALQKVKIEVNESGTVASSSTAVIVSARMAPEEIIMDRPFLFV
VRHNPTGTVLFMGQVMEP
O. Dog PAI-1 derived amino acid sequence
(leader sequence underlined)
MGSSHHHHHHSSGLVPRGSHMSYSYQTRAAGLATDFGVKVFQEVARAS
KDRNMVFSPYGVASVLAMLQLTTAGETCRQIQEAMQFQIDEKGMAPAL
RQLYKELMGPWNKDEISTADAIFVQRDLKLVHGFMPHFFRLFRTTVKQ
VDFSEVERARFIVNDWVKRHTKGMIGNLLGRGAVDQLTRLMLVNALYF
NGQWKTPFPESGTHHRLFHKSDGSTVSVPMMAQTNKFNYTEFSTPSGH
YYDILELPYHGDTLSMFIAAPYEKEVPLSALTNILDAQLISQWKGNMT
RQLRLLVLPKFSLETEVNLRRPLENLGMTDMFRPNLADFSSLSNQEVL
YVSRALQKVKIEVNESGTVASSSTAIIVSARMAPEEIIMDRPFLFVVR
HNPTGTVLFMGQVMEP

### Example 6

### Identification of Essential Point Mutations for Potency on Rodent PAI-1 by Site-Directed Mutagenesis and Cell-Free Chromogenic Assays

In an attempt to identify which amino acid substitutions in the VH and VL domains were responsible for the significant potency improvements measured for some of the variants tested above, the sequences of all scFvs with IC₅₀s within a 1000-fold ratio in the human and rat chromogenic assays were aligned and compared. Most of them were altered in the two mutation hot-spots in VHCDR2 and VLCDR1. For the variants isolated from the initial error-prone PCR (EP) library, the glycine for aspartic acid substitution was predominant at position 55 of VH. The amino acid at position 32 of VL however was mutated more often to asparagine and glutamic acid.

In addition to these hot-spots, two other important amino acid changes were revealed. Lysine at position 50 in VLCDR2 was changed to arginine in many clones and glycine at the Vernier framework position 66 of VL (Foote, J., et al. J Mol Biol, 1992. 224: p. 487) was replaced by arginine in variants with IC₅₀s in the human and rodent cell-free chromogenic assays within a 2-fold ratio.

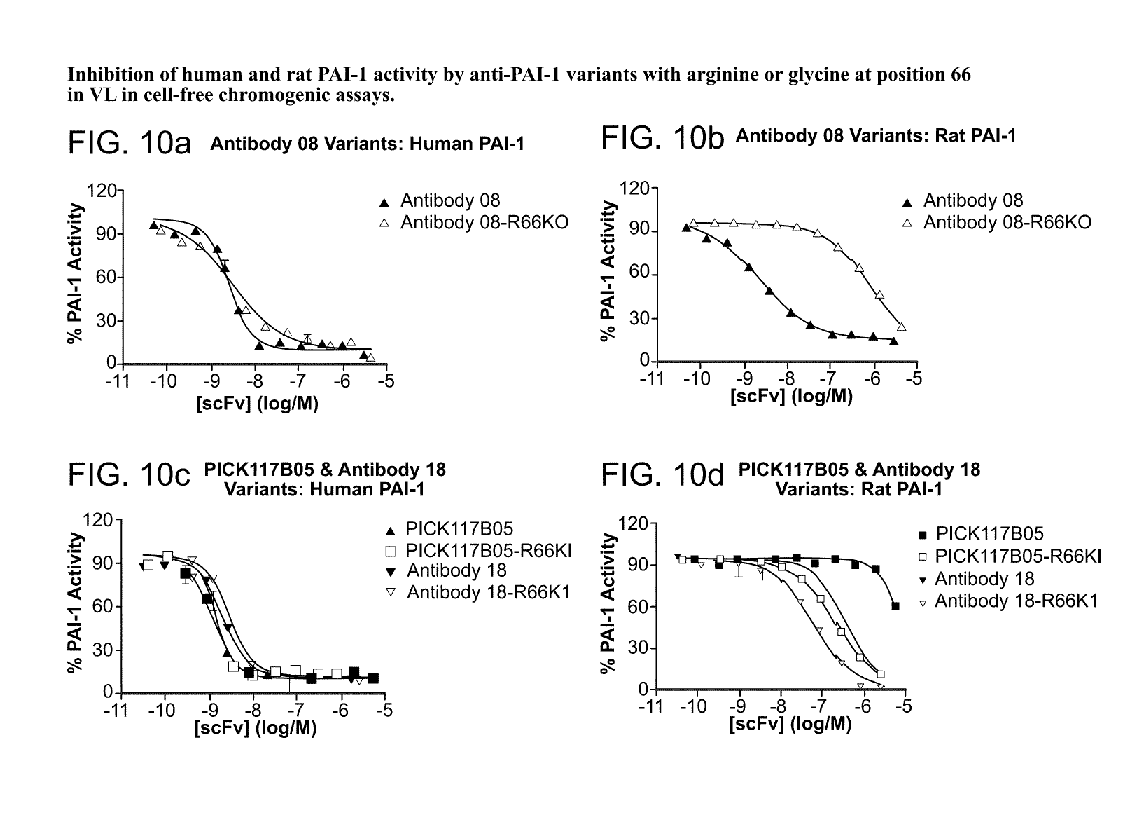

To substantiate the latter observation, the following mutational analyses were carried out on cDNA encoding the scFv (Stratagene QuikChange® kit). Antibody 08, equipotent in human, cyno and rodent cell-free chromogenic assays (see Example 1), was mutated to reverse the engineered arginine 66 back to glycine. The resulting variant Antibody 08-R66KO scFv was then retested in the chromogenic assays. Its IC₅₀ in the human assay was essentially unchanged, with an IC₅₀ of 4.3 nM as compared with 3.2 nM for Antibody 08 (FIG. 10a). The IC₅₀ in the rat assay however increased about 300-fold to 920 nM for the mutated variant compared with 3 nM for the non-mutated Antibody 08 scFv (15b).

The importance of this arginine 66 of VL for cross-reactivity was further confirmed by introducing it into PICK117B05 and Antibody 18, which both still contained glycine at this position. Prior to mutation these scFv had IC₅₀ values in the human assay of 0.8 nM and 1.3 nM, respectively (FIG. 10c). In the rat assay however, PICK117B05 scFv gave an approximately 1000-fold higher IC₅₀ value of greater than 1 μM and Antibody 18 scFv gave an approximately 180-fold higher IC₅₀ value of 240 nM when compared with data from the human assay (FIG. 10d). After introduction of arginine for glycine at position 66 of their VLs, the potencies remained the same in the human assay. The IC₅₀s were 2.7 nM for PICK117B05-R66KI and 4.5 nM for Antibody 18-R66KI. The substitution however had a beneficial effect on their inhibitory activity on rat PAI-1, with the respective IC₅₀s dropping over 10-fold to 220 nM for PICK117B05-R66KI and over 5-fold to 63 nM for Antibody 18-R66KI, resulting in human to rat IC₅₀ ratios of about 80- and 14-fold, respectively.

### Example 7

### Affinity of Anti-PAI-1 Antibodies for Human and Rat PAI-1 as Determined by BIAcore

The cross-reactivity of the anti-PAI-1 antibodies to human and rodent PAI-1 was further confirmed by affinity determination. Surface plasmon resonance measurements using a BIAcore 2000 Biosensor (BIAcore AB) were performed essentially as described by Karlsson et al. (Karlsson, R., et al. J Immunol Methods, 1991. 145: p. 229). In brief, antibodies were coupled to CM5 sensorchips using an amine coupling kit (BIAcore) at a surface density of approximately 500 RU and a serial dilution (between 25 nM and 400 pM) of human and rat PAI-1 (both Molecular Innovations) in Hepes Buffered Saline/1 mg bovine serum albumin was passed over the sensorchip surface. The resulting sensorgrams were evaluated using BIA evaluation 3.1 software to provide kinetic data.

The dissociation constants (KD) for the tested anti-PAI-1 IgGs under the chosen conditions were between 10 pM and 120 pM on active human PAI-1. The on-rates (kon) were in the range of 2.2×106 M-1s-1 and 1.3×106 M-1s-1, the off-rates (koff) were between 1.5×10-4 s⁻¹ and 2.3×10-5 s⁻¹. The affinities on active rat PAI-1 were within about a 10-fold ratio to human for each antibody. The KDs were between 110 and 300 μM with kons ranging from 1×106 M-1s-1 to 2.5×106 M-1s-1 and /k_{off}s from 2.1×10⁻⁴ s⁻¹ to 7.2×10⁻⁴ s⁻¹. These values confirmed the cross-reactivity of the antibody molecules to human and rat PAI-1.

In a further experiment, the affinity of the anti-PAI-1 antibody, Antibody 08 to human glycosylated PAI-1, human stable mutant PAI-1, cyno PAI-1 and rat PAI-1 was determined by BIAcore. Surface plasmon resonance analysis using a BIAcore T100 Biosensor (GE Healthcare) was performed using a modification of experimental conditions described above. Antibody 08 was coupled to CM3 sensorchip using amine coupling at approximate surface densities of 100 and 500 RU and a serial dilution (between 6.25 nM and 50 pM) of glycosylated human, stable mutant human, rat PAI-1 (all Molecular Innovations) and cyno (recombinantly expressed in-house and purified from E. coli) in HBS-EP+ (Biacore) was passed over the sensorchip surface. The sample was injected for 120s at 30 ul/min (or higher) and the dissociation period monitored for 800s per cycle. Stable regeneration of the chip surface was achieved by paired injections of 10 mM Glycine pH2. Affinity determination was performed at both 25° C. and 37° C. The resulting sensorgrams were double reference-subtracted against a buffer blank and evaluated using BIA evaluation software to provide kinetic data by fitting to a 1:1 Langmuir model. Fits were assessed for quality and evidence of mass transport limitation using the returned fitting parameters & residuals.

A very high affinity interaction was observed with on-rates (kₒₙ) of approximately 1×10⁶ M⁻¹s⁻¹ and off-rates (k_{off}) of approximately 1×10⁻⁵s⁻¹. Antibody 08 consistently showed an affinity of less than 20 pM for human and cyno PAI-1. No significant difference in the mean of multiple kinetic results was observed between 25° C. (Table 6a) and 37° C. (Table 6b).

### Example 8

### Cross-Reactivity of Anti-PAI-1 Antibodies to Active PAI-1 Species Variants and Selectivity Over Latent Human PAI-1 and Other SERPINs as Measured by Competition Binding Assay

The relative cross-reactivity of the anti-human PAI-1 antibody molecules to the various PAI-1 species variants identified in the functional chromogenic and plasminogen activation assays (see Examples 1 and 3) was further assessed biochemically by competition binding assays. IgG binding to active human PAI-1 was tested against mouse, rat and rabbit PAI-1 using the competition binding assay.

As shown in FIG. 11a, Antibody 16, for example, bound to rat PAI-1 with an apparent affinity of only 4.8-fold lower than for human under these conditions. The ratios to mouse and rabbit were 14.4 and 7.9, respectively. The binding to latent PAI-1 was about 11-fold weaker and the binding to glycosylated PAI-1 produced in house by Chinese hamster ovary (CHO) cells (Stromqvist, M., et al., 1994, supra) was within about 2-fold of binding to bacterially-derived antigen. In general, the data from the previous examples was confirmed for all tested clones.

Data obtained in the competition binding assay are summarised in Table 7, which shows ratios of IC₅₀s of selected antibodies in respect of glycosylated active human PAI-1, latent human PAI-1, rat PAI-1, mouse PAI-1 and rabbit PAI-1 as compared with non-glycosylated active human PAI-1 (“wt”). These values thus represent fold differences in potency of the binding members for neutralising these PAI-1 molecules as compared with potency for neutralising non-glycosylated active human PAI-1. In addition, the PAI-1 neutralising antibodies were assessed for their selectivity for active human PAI-1 over other related SERPINs using the same assay. As shown in FIG. 11b for Antibody 16, no binding could be detected to PAI-2 and PAI-3 (both MRC Cambridge), antithrombin III and α1-antitrypsin (US Biological), α1-antichymotrypsin (Research Diagnostics) and α2-antiplasmin (American Diagnostica) under the chosen assay conditions.

### Example 9

### Inhibition of PAI-1:tPA Complex Formation by Anti-PAI-1 Antibodies as Assessed by SDS-PAGE Analysis

As described elsewhere herein, incubation of tPA with active PAI-1 leads to the formation of a covalent PAI-1:tPA complex upon the formation of an ester bond between the active site serine of tPA and the P1 residue of the PAI-1 reactive centre loop (Gils, A., et al. Thromb Haemost, 2003. 90: p. 206). This covalent complex can be demonstrated by SDS-PAGE analysis as a higher molecular weight band compared with PAI-1 and tPA alone. The antibodies described herein can be shown to inhibit the formation of the complex by using this method. Both, E. coli-derived as well as CHO-derived, and thus glycosylated, active human PAI-1 could be prevented from binding to tPA by the presence of IgG as demonstrated by the absence of the higher molecular weight band. No enhanced formation of a cleaved and thus lower molecular weight form of PAI-1 was observed following antibody incubations, indicating that these antibodies do not act by inducing a substrate conformation. On the contrary, the data supports a mechanism of inhibition involving prevention of the formation of the Michaelis-Menten complex.

### Example 10

### PEPSCAN-Based Peptide Scan

A candidate IgG antibody molecule selected from antibodies 1 to 27 was screened against 3310 peptides representing both linear overlapping peptides and a large number of conformationally restrained peptides representing short regions of amino acid sequence derived from the human PAI-1 sequence SEQ ID NO: 124. The synthetic peptides were covalently coupled to a solid phase credit card format to allow screening of antibody binding. Binding of the IgG was detected by incubation with an anti-human antibody peroxidase conjugate using 2,2′-azino-di-3-ethylbenzthiazoline sulfonate and hydrogen peroxide as the colourimetric detection reagents. The antibody was tested at two concentrations (0.01 μg/ml and 0.05 μg/ml).

Analysis of the detection signals indicated that eight of the top 10 binding values obtained at the 0.01 μg/ml antibody concentration were derived from peptides containing the motif GHYYD. The eight are marked with * in Table 8. The GHYYD motif is located at amino acid residues 218-222 in the mature PAI-1 sequence SEQ ID NO: 124.

The two peptides identified in the top ten binding signals which did not contain the GHYYD motif were not considered to be relevant as they correspond to sequences which would be found buried in the PAI-1 molecule and may therefore represent non-specific binding events.

The peptide binding data was also analysed by calculating the average binding signal for all peptides analysed at the 0.01 μg/ml antibody concentration and for each peptide a signal/average background signal ratio was calculated. A ratio of 3 or greater was considered a positive signal. Table 9 shows all peptide sequences which gave a ratio of 3 or greater. The highest binding signal ratios were obtained for peptides containing the GHYYD motif within the context of a constrained peptide. Additional peptides which gave ratios above three included motifs derived from both loops 204-210 and 267-273 (numbering according to the mature PAI-1 sequence SEQ ID NO: 124) with the highest ratio being derived from the peptide CQTNKFNYCTRLPRLLC (SEQ ID NO: 143). This peptide is indicated by * in Table 9. This result is consistent with the regions QTNKFNY (SEQ ID NO: 157) and TRLPRLL (SEQ ID NO: 158) also contributing to the epitope.

### Example 11

### Mutagenesis of Putative PAI-1 Epitope Regions and SDS-PAGE Analysis of Inhibition of PAI-1:tPA Complex Formation for Mutant PAI-1

Using site-directed mutagenesis, the Arg corresponding to position 271 of mature human PAI-1 (SEQ ID NO: 124) was mutated to Ser, changing the motif TRLPRLL (SEQ ID NO: 158) to TRLPSLL (SEQ ID NO: 188). Mutagenesis was performed on a stable mutant form of human PAI-1 having sequence SEQ ID NO: 190 (Berkanpas, M. B., Lawrance, D. A. and Ginsburg, D. 1995. EMBO Journal, 14, 2969-2977), to form SEQ ID NO: 191 containing the Arg to Ser point mutation. Arg271 of mature human PAI-1 corresponds to Arg292 in SEQ ID NO: 190.

SDS-PAGE analysis was performed as described herein, except that the Arg292Ser mutant PAI-1 (SEQ ID NO: 191) was used. The mutant PAI-1 contains the point mutations Asn150His, Lys154Thr, Gln319Leu and Met354Ile (residue numbering per SEQ ID NO: 124) compared with mature human PAI-1. These mutations increase stability of the mutant form in the active conformation, so that it converts to the latent form more slowly than wild-type PAI-1. Use of the stable mutant may confer practical advantages in terms of handling of the proteins during the assay method. The mutant form also bears an N-terminal His tag.

SDS-PAGE analysis of inhibition of the Arg to Ser mutant PAI-1:tPA complex formation indicated that the candidate antibody did not block complex formation. Data on affinity of antibody binding to Arg to Ser mutant PAI-1 were obtained using BIAcore, and indicated that affinity of a candidate antibody of for binding the mutant PAI-1 was lower than for binding wild-type human PAI-1.

These data indicate that binding to the motif TRLPRLL (SEQ ID NO: 158) contributes to the mechanism of inhibition of the antibodies disclosed herein. The data further indicate that the Arg residue at position 271 of mature human PAI-1 is structurally important for antibody:antigen recognition, suggesting that binding members of the disclsoure may bind R271 of mature human PAI-1.

### Example 12

### X-Ray Crystal Structure Determination of the PAI-1 Antibody Complex

A structure of PAI-1 bound to Antibody 08 Fab was determined by X-ray crystallography. The methods of Fab production, PAI-1 protein preparation, crystallisation and crystallography are generally well-known. Crystals of the complex between a stability mutant of PAI-1 (SEQ ID NO: 190) and Antibody 08 Fab fragment were obtained by known methods. The crystal form of the complex was that of thin plates with the monoclinic space group P21. Complete diffraction data initially to 3.2 Å and subsequently to 2.9 Å resolution were obtained. The structure could be solved by Molecular Replacement, using search models derived from previously determined structures of the PAI-1 stability mutant and Fab fragment to enable the correct positioning of all the PAI-1 and the Fab molecules present in the crystallographic asymmetric. There were four PAI-1 molecules and four Fab fragments in the asymmetric unit corresponding to four independently observed complexes and epi/paratopes.

In the crystal structure each of the four complexes has a unique set of crystal packing interactions. Two of the Fab constant domains and the lower part of one of the PAI-1 molecules have very few stabilising interactions in the crystals lattice. This results in partial disordering as indicated by the high average B-factor for those domains. These regions are flexible and adopt different orientations throughout the crystal such that the electron density is averaged out. In contrast, all the Fab variable domains and their CDRs as well as all of the interacting area of the PAI-1 molecules are well ordered. All four complexes can be used to validate the binding mode and epi/paratope. Within the accuracy expected from a 2.9 Å structure solved by molecular replacement the four observed epi/paratopes appear to be nearly identical. The presence of two water molecules (potentially crucial for binding) is inferred although not proven by the structure, since that requires a resolution better than 2.5 Å.

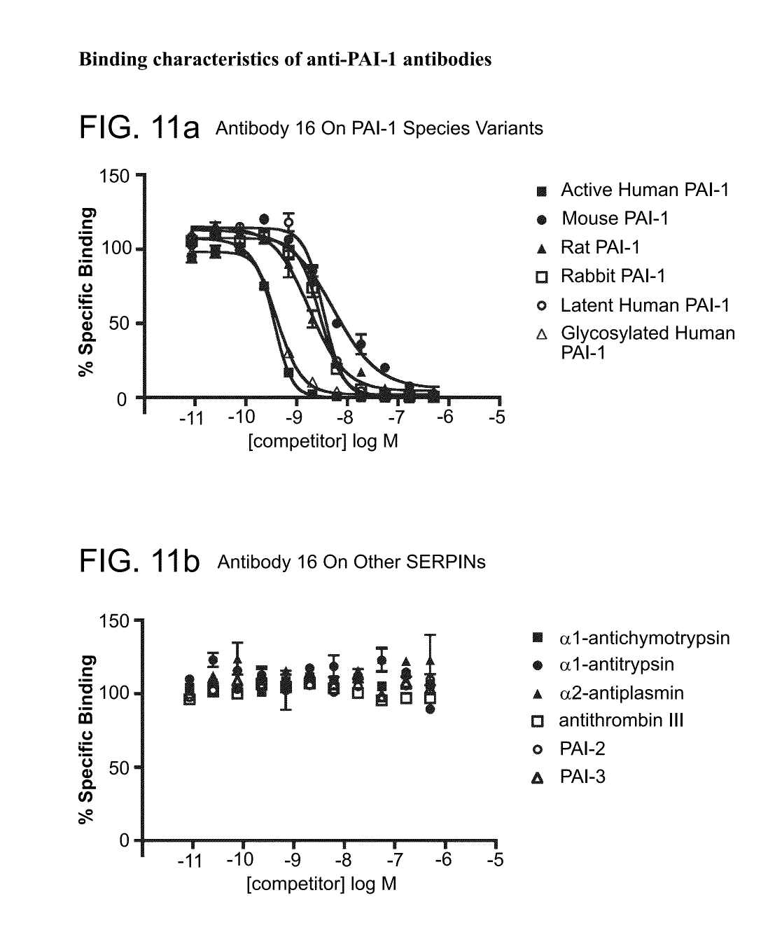

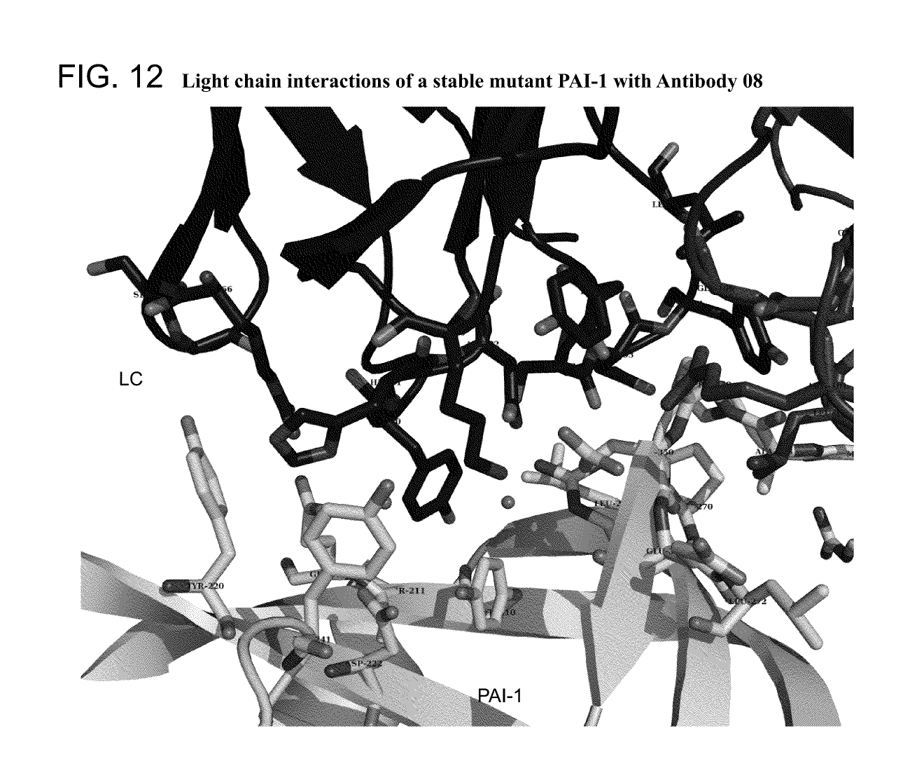

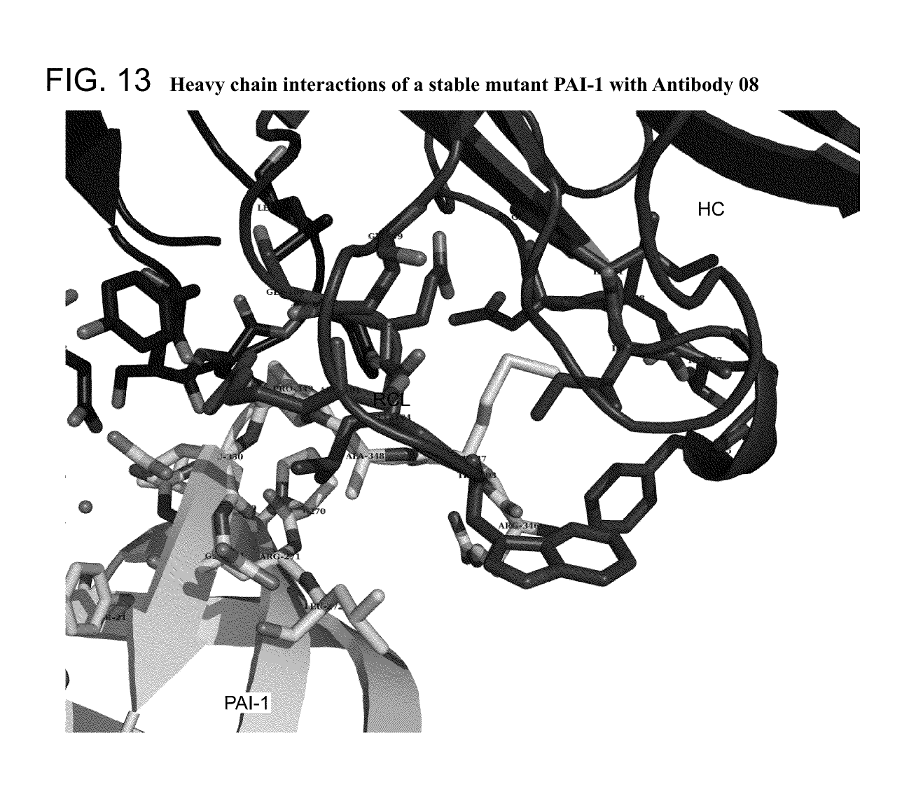

The crystal structure allows the epitope and paratope interactions between PAI-1 and Fab to be examined in atomic detail. These are shown in Table 10. Interactions within 3.2 Å are polar, for example H bonds. Interactions within 4.0 Å are non-polar and interactions within 5.0 Å are closely neighbouring. Whilst the stability mutant of PAI-1 (SEQ ID NO: 190) was used in this work, the residue numbering of PAI-1 given in this example and Table 10 is that of mature human PAI-1 (SEQ ID NO: 124). For example, Arg271 of mature human PAI-1 corresponds to Arg292 in SEQ ID NO: 190. The numbering for heavy and light chain residues given in this example and Table 10 is Kabat numbering, with the residue numbering as determined by crystal sturcture analysis given in parenthesis, where different. The H-bonding network comprises a bond between residue Arg 271 of mature human PAI-1 and light chain residues Asn 32 and Ser 92, residue Ala 348 of PAI-1 and heavy chain residue Glu 100A (105) and residue Glu 350 of PAI-1 with heavy chain residue Arg 97 (101) and light chain residue Lys 50. Interactions between PAI-1 and the light chain and heavy chain are shown in FIGS. 12 and 13, respectively.

A salt bridge is present between residues Glu 212 of human mature PAI-1 and light chain residue 66. Mutation studies in which residue Glu 212 of PAI-1 was mutated to Val 212 resulted in a decrease of binding activity between PAI-1 and Antibody 08 due to loss of this salt bridge.

Residue Arg 271 of human mature PAI-1 forms a H-bond within the main chain of light chain residue Ser 92. Residue Ala 348 of PAI-1 interacts with the main chain of heavy chain residue Glu 100A (105). Buried in the interface with heavy chain residue Leu 100 (104) are the PAI-1 residues Ala 348, Pro 349 and Glu 350. PAI-1 residue Glu 350 bridges the heavy and light chains of Antibody 08 forming interactions with heavy chain residue Arg 97 (101) and light chain residue Lys 50.

All the amino acids from Antibody 08 that contribute to binding human mature PAI-1 are from the heavy and light chain CDRs, except for residues Arg 66, Ser 67 and Gly 68 (RSG 66-68) of the light chain of Antibody 08. The paratope residues of Antibody 08 are underlined in the table below:

| | SEQ | | | SEQ | |
|---|---|---|---|---|---|
| CDR | ID NO: | Amino Acid | CDR | ID NO: | Amino Acid |
| L1 | 176 | RASEGIYHNLA | H1 | 187 | SYAIS |
| L2 | 184 | KASSLAS | H2 | 161 | GIIPTFGTANYAQKFQG |
| | | (RSG) | | | |
| L3 | 186 | QQYSNYPLT | H3 | 171 | ERRQWLEGHFDY |

Superimposition with Latent PAI-1

The active form of PAI-1 mainly reacts with binding agents via the reactive centre loop, which is not present in the latent form. X-ray crystallography studies have shown that active PAI-1 binds Antibody 08 with higher affinity due to H-bond interactions between the reactive centre loop of PAI-1 and Antibody 08 Fab and buried residues 347-350 of the reactive centre loop. This structural information explains the reduced binding between Antibody 08 and latent PAI-1.

### A. Glycosylation Sites

The sequence of human mature PAI-1 contains three possible N-glycosylation sites consistent with Asn-X-Thr/Ser motif: N209, N265 and N329. X-ray crystallography studies have confirmed that sites N209 and N265 are utilised for glycosylation whilst the N329 site is not utilised. These glycosylation sites are unlikely to interfere with antibody binding, even though N209 is next to the epitope residue Y210 in sequence. Structural analysis has shown that these glycosylation sites face away from the reactive centre loop of PAI-1 and the side-chain of N209 is oriented away from the interaction interface.

B. Epitope Comparison with Rodent PAI-1

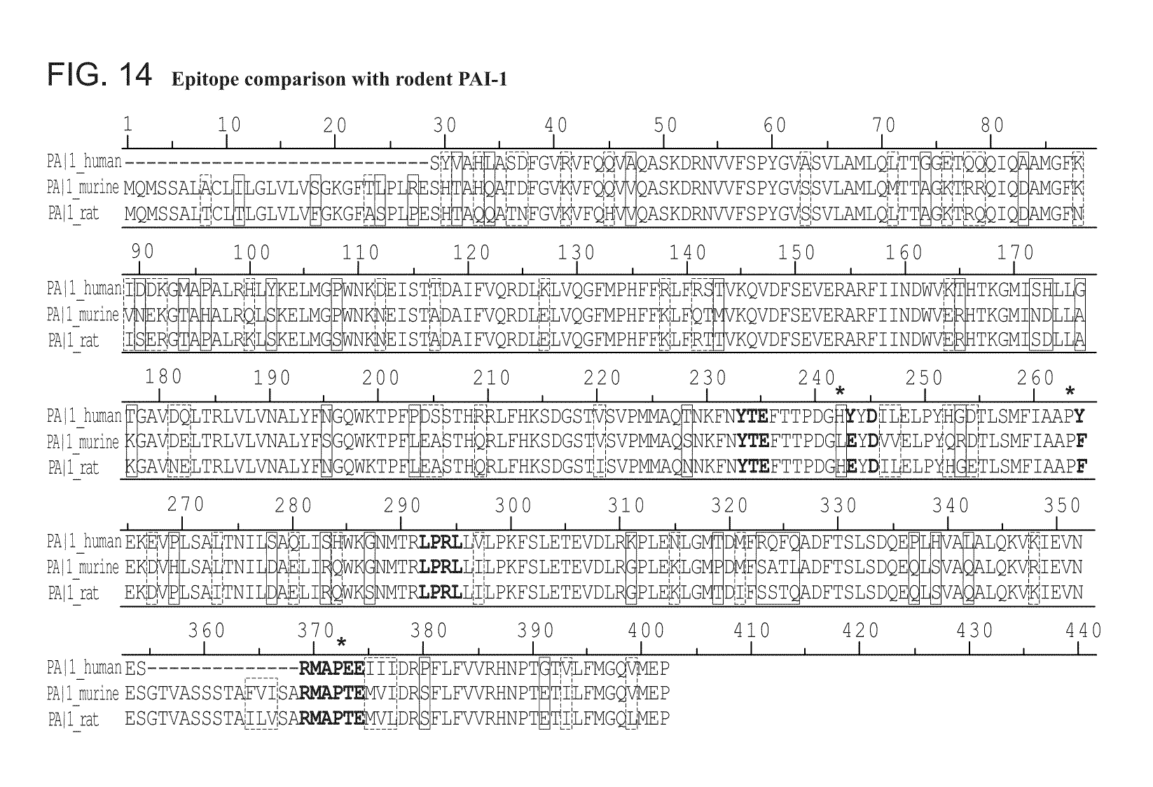

Alignment of human, murine and rat PAI-1 sequences (SEQ ID NOs: 119-121) indicated three amino acid differences in the PAI-1 epitope between human and rodent PAI-1 (see FIG. 14). These epitope amino acids are Y220E, Y241F and E350T, with the changes indicated from human to rodent sequence. As shown by our structural analysis Y220 and Y241 are involved in binding to the light chain and E350 to the heavy chain of Antibody 08. The difference in affinity of Antibody 08 of approximately 10 fold between human and rodent PAI-1 can be accounted for by these amino acid changes in the epitope. In particular, the E350T mutation breaks two H-bonds with R97 (101) and K50, as indicated by structural analysis.

C. Epitope Comparison with Anti-PAI-1 Antibodies

A comparison was made between the epitopes of the anti-PAI-1 antibodies scFv-56A7C10 (Bijnens, A. P. et al., 2001 J Biol. Chem., 276, 44912-44918: Novoa de Armas, H., et al., 2007 Structure 15, 1105-1116), which binds active PAI-1, MAI-12 (Dupont et al., 2006 J Biol. Chem., 281(47), 36071-36081), which binds latent and active PAI-1, H4B3 (Dupont et al., 2006, supra), which binds latent PAI-1 and Antibody 08. The results are shown in Tables 12 and 13. Table 12 shows that three of the PAI-1 epitope residues recognised by MAI-12 and four of the epitope residues recognised by H₄B3 are also recognised by Antibody 08. Table 13 shows that Antibody 08 has 13 amino acid residues in common with the epitope recognised by scFv-56A7C10, which comprises 32 amino acid residues. However, as reflected in the table, there are numerous difference in the residues recognized by Antibody 8 compared to those recognized by these other antibodies.

### D. Polymorphism Changes

As mentioned earlier, a number of polymorphisms have been found in the coding sequence of SERPINE1 (PAI-1), five of which change the amino acid sequence (see Table 1). In order to determine whether these amino acid changes were likely to have an effect on antibody binding, the location of these polymorphisms were studied in relation to the crystal structure of PAI-1/Antibody 08 Fab. Residues 1-23 of human PAI-1 (SEQ ID NO: 119) are part of the signal peptide and two of the amino acid changes (A15T and V17I), which result in polymorphisms, are found in this signal peptide. Furthermore, these two amino acid changes are responsible for the highest allele frequency for European, African American, Chinese, and Asian populations. These two amino acid changes do not impact on antibody binding as they are not present in active PAI-1. In addition the amino acid change H25P is not visible in the crystal structure electron density. The epitope region is predominantly at the C-terminus of the structure and although the H25P effect on antibody binding has not been determined, it is unlikely to interfere. Thus, Antibody 8 is likely to bind with substantially the same affinity to the PAI-1 proteins resulting from these polymorphisms.

The three remaining amino acid changes resulting in PAI-1 polymorphisms (R209H, D216 and T255N; SEQ ID NO 119) are visible in the crystal structure. However, the results of our structural modelling work have shown that these amino acids are not structurally part of the identified epitope on PAI-1 and therefore are unlikely to affect PAI-1/Antibody 08 binding interactions. Therefore, Antibody 08 binds to PAI-1 at an epitope that remains unchanged for most polymorphisms identified, i.e., antibody binding will occur irrespective of the population targeted.

### Example 13

### Mutagenesis Studies to Confirm Crystal Structure Analysis

A number of the amino acids that are part of the structurally identified epitope were subject to site-directed mutagenesis to determine the effect of the mutation on the PAI-1/antibody Fab binding. A panel of PAI-1 mutant proteins was constructed that each contained a single amino acid mutation compared to the wild-type (wt) sequence. The binding of these mutants to Antibody 08 was then evaluated in biochemical competition assays as described previously, with changes in the concentrations of antibodies and PAI-1 mutant proteins where required. Generation of human PAI-1 and the PAI-1 mutant proteins is detailed in the Materials and Methods section below.

Data on affinity of antibody binding to PAI-1 mutant proteins were obtained using BIAcore, and indicated that affinity of a candidate antibody of the disclsoure for binding the mutant PAI-1 proteins was lower than for binding a stable mutant human PAI-1 (Table 14).

These data indicate that binding to a specific epitope amino acid residue contributes to the mechanism of inhibition of the antibodies disclosed herein. The data further indicate that the Tyr residue at position 210, Glu residue at position 212, Thr residue at position 220, Tyr residue at position 241 and Arg residue at position 271 of mature human PAI-1 (SEQ ID NO: 124) are structurally important for antibody:antigen recognition, confirming that binding members of the disclsoure may bind these amino acid residues of mature human PAI-1.

### Assay Materials and Methods for the Examples

The detailed methods for assays described in the foregoing examples are provided below. Note: IgG used in the assays described herein were produced by expression in HEK cells.

Chromogenic Assay (Human)

The assay was carried out at room temperature in 96-well assay plates (Costar) in 50 μl 100 mM Tris pH 8.4, 106 mM NaCl, 0.01% Tween 80. Recombinant human PAI-1 (Molecular Innovations) was dispensed into the wells to a final concentration of 5 nM, corresponding to its experimentally determined EC80 value. Purified scFv or IgG preparations were quantified and a series of dilutions were added to the wells. After 10 minutes incubation, human tPA (Actilyse, Boehringer Ingelheim) was added to a final concentration of 2.14 nM, and the samples were incubated for a further 5 minutes. Finally, the chromogenic substrate (S2288, Quadratech) was added to a final concentration of 920 μM, corresponding to the experimentally determined Kₘ value. After 1 hour 15 minutes incubation, colour generation was measured as absorbance at 405 nm using an EnVision plate reader (PerkinElmer). Raw absorbance data was first converted to % PAI-1 activity where 100% was taken from control wells containing PAI-1 but no inhibitor and 0% was taken from control wells containing no PAI-1. Resultant data was then analysed using Prism curve fitting software (Graphpad) to determine IC₅₀ values.

### A. Adaptations to Chromogenic Assay for Other Species

PAI-1 was used at experimentally determined EC80 concentrations, as shown in the table below. The chromogenic substrate (S2288, Quadratech) for all tPA was added to final concentrations corresponding to the experimentally determined Km values for the respective tPA, as shown in the table below.

| | human | rat | mouse | cynomolgus | dog |
|---|---|---|---|---|---|
| tPA | human tPA | rat tPA | mouse tPA | human tPA | human tPA |
| | (Actilyse, | (Molecular | (Molecular | (Actilyse, | (Actilyse, |
| | Boehringer | Innvtns.) | Innvtns.) | Boehringer | Boehringer |
| | Ingelheim) | | | Ingelheim) | Ingelheim) |
| tPA concn. | 2.14 nM | 1.34 nM | 1.63 nM | 2.14 nM | 2.14 nM |
| PAI-1 | human PAI-1 | rat PAI-1 | mouse PAI- | cyno PAI-1 | (bacterially- |
| | (Molecular | (US Biol.) | 1 (US | (bacterially | derived |
| | Innvtns.) | | Biological) | derived, | active dog |
| | | | | produced in | PAI-1 |
| | | | | house) | (produced in |
| | | | | | house) |
| PAI-1 | 5 nM | 2 nM | 6 nM | 5 nM | 55 nM |
| final | | | | | |
| concn. | | | | | |
| tPA | 920 μM | 414 μM | 452 μM | 920 μM | 920 μM |
| substrate | | | | | |
| (S2288) | | | | | |
| final | | | | | |
| concn. | | | | | |

### B. HT-1080 Cell Based Plasminogen Activation Assay

HT-1080 cells (European Collection of Cell Cultures, ECACC) were seeded in 96-well flat-bottomed tissue culture assay plates (Costar) at 2×10⁴ cells/well. They were cultured in Minimum Essential Media (MEM) with Earles salts and L-glutamine, 10% (v/v) heat inactivated Australian foetal bovine serum (FBS), 1% (v/v) MEM-non-essential amino acids (without L-glutamine) (all from Invitrogen) for 24 hours in a humidified atmosphere at 37° C. and 5% CO₂. Prior to use in the assay, the media was aspirated and the cell monolayers were washed twice with 200 μl phenol red-free Hank's balanced salt solution (HBSS, Invitrogen).

In parallel, dilution series of purified anti-PAI-1 scFv or IgG were prepared in HBSS and pre-incubated with human recombinant PAI-1 (10 nM final concentration, Molecular Innovations) for 30 minutes at room temperature. 100 μl were then transferred to the pre-washed cells, and the reaction was started by adding human plasma Lys-plasminogen (Calbiochem) and the uPA/tPA-substrate S-2251 (H-D-Val-Leu-Lys-p-nitro-anilide, Chromogenix) to final concentrations of 5 μg/ml and 0.2 mM, respectively. The cells were incubated for up to 3 hours at 37° C. and 5% CO₂, during which the generation of coloured uPA/tPA-product was monitored by measuring absorbance at 405 nm using a Victor plate reader (PerkinElmer). Absorbance values were analysed by performing an Area Under Curve (AUC) analysis for each concentration of anti-PAI-1 scFv or IgG and normalised for control rates without antibody (100% in the absence of PAI-1, 0% in the presence of PAI-1). The data was plotted as % of the control response against log (concentration of scFv or IgG) and IC₅₀ values were determined using Prism curve fitting software (Graphpad).

### C. Competition Binding Assay

The assay was carried out at room temperature. Purified IgG antibodies were adsorbed to 96-well flat-bottomed Maxisorp assay plates (Nunc) in 50 μl PBS at a concentration which gave a significant signal when biotinylated active human PAI-1 (Molecular Innovations, biotinylated in house) was added at approximately its predetermined KD for that particular IgG. Excess IgG was washed away with 0.1% Tween 20 in PBS (PBS-T20/0.1) and the wells were blocked with 300 μl PBS containing 3% (w/v) dried milk powder (Marvel) for 1 hour. A dilution series of competitor mouse, rat or rabbit PAI-1 (all US Biological) was prepared in PBS, starting at a concentration of ≧200-fold the KD value of the interaction between biotinylated active human PAI-1 and the respective IgG. Insulin was included as an irrelevant antigen control, unbiotinylated active human PAI-1 as a positive control. To this series, an equal volume of biotinylated recombinant human PAI-1 at a concentration of approximately 2-fold the KD was added (resulting in a series starting at a ratio of competitor antigen:biotinylated active human PAI-1 of ≧100:1). These mixtures were then transferred onto the blocked IgG. After 1 hour equilibration, unbound antigen was removed by washing 3 times with 300 μl PBS-T20/0.1, while the remaining biotinylated human PAI-1 was detected by streptavidin-Europium3+ conjugate (PerkinElmer). Fluorescence was measured on an EnVision plate reader (PerkinElmer). Raw absorbance data was first converted to % PAI-1 specificity (100% was determined from control wells containing biotinylated human PAI-1 but no competitor, 0% was from wells containing biotinylated human PAI-1 and a 50-fold excess of unbiotinylated human PAI-1). Resultant data were analysed using Prism curve fitting software (Graphpad) to determine IC50 values. Due to the different affinities of each IgG to PAI-1, and therefore the different assay conditions used, results were expressed as fold difference in IC50s between active human PAI-1 and competing antigens. This enables comparison of different IgGs.

### D. Protocol for SDS-PAGE Analysis of Inhibition of PAI-1:tPA Complex Formation

2 μg recombinant active human PAI-1 derived from E. coli (Molecular Innovations) or produced in house by Chinese hamster ovary (CHO) cells (Stromqvist 1994) were incubated for 15 minutes with 1 μg human tPA (Actilyse, Boehringer Ingelheim) at 37° C. in a final volume of 25 μl PBS, pH 7.5. Anti-PAI-1 antibodies were tested by pre-incubating 20 μg IgG with PAI-1 for 10 minutes prior to addition of tPA to initiate the reaction. The reaction was terminated by the addition of 8 μl SDS-PAGE buffer followed by heating at 90° C. Aliquots of the samples were analysed on a 12% Bis-Tris Novex NuPAGE gel run in MOPS buffer (Invitrogen). Proteins were detected using Coomassie blue stain.

### E. Peptide Binding Scan (PEPSCAN) Method

Overlapping linear 15-mer and constrained (looped) 8-, 10- and 12-mer synthetic peptides derived from the mature human PAI-1 sequence were synthesised and screened using credit-card format mini-PEPSCAN cards (455-well-plate with 3 ul wells) as described previously (Slootstra-J W; Puijk-W C; Ligtvoet-G J; Langeveld-J P; Meloen-R H (1996). Mol-Divers. 1: 87-96). In addition, a set of double looped synthetic peptides were synthesised. In total 3310 peptides were synthesised. Binding of antibodies to each peptide was tested in a PEPSCAN enzyme-linked immuno assay (ELISA). 455-well credit card-format polypropylene cards containing the covalently linked peptides were incubated with antibody samples at either 0.05 or 0.01 μg/ml. After washing, the bound antibody was detected with an anti-antibody peroxidase conjugate and the peroxidase substrate 2,2′-azino-di-3-ethylbenzthiazoline sulfonate (ABTS) and hydrogen peroxide. The colour development was quantified with a CCD-camera and an image processing system.

### F. Production and Analysis of Antibody 08 Fab for X-ray Crystallography

Purified Antibody 08 was digested to generate Fab fragments as follows:
a digest buffer of 30 mM DL-cysteine hydrochloride dissolved in GIBCO PBS (Invitrogen) was prepared.
papain from papaya latex (Sigma) was reconstituted in digest buffer to give a 10 mg/ml solution and kept at room temperature for a minimum of 30 minutes before use.
cysteine was added to IgG to give a 30 mM solution.
papain was added to the IgG at a ration of 1 mg papain to 100 mg IgG.
the digest was terminated after 4.5 hours by the addition of 0.5 M iodoacetamide (Sigma) to give 50 mM iodoacetamide in the final digest mixture.

The Fab was then purified using Q Sepharose (GE Healthcare), buffer exchanged into PBS (Gibco) pH 7.2 and concentrated to approximately 10 mg/ml for use in X-ray crystallography, which is described below.

### G. X-Ray Crystal Structure Determination: Protein Preparation, Crystallisation and Crystallography

Purified protein of a stable quadruple mutant of PAI-1 (Berkanpas, M. B., Lawrance, D. A. and Ginsburg, D. 1995. EMBO Journal, 14, 2969-2977) (SEQ ID NO: 190) was obtained from Jim Huntington, University of Cambridge, which had been produced according to the protocol referred to in Zhou et al (Zhou, A. et al., 2003 Nature Struc. Biol., 10(7), 541-544).

The material used for crystallisation was formulated by mixing the proteins in their original buffer in a 1:1 ratio followed by a rapid buffer exchange step to a solution of 15 mM ammonium acetate pH 7.4 using a PD-10 column (GE Healthcare). Prior to the crystallisation the complex was concentrated to 3-9 mg/ml using a 500 μl 30 kDa molecular weight cut-off centrifugal concentrator (Millipore Biomax) that had previously been washed with 1 ml of the same buffer solution.

Sitting drop vapour diffusion crystallisation screening was carried out at 20° C. using Greiner low profile trays and 150 nl+150 nl drops. Crystals representing mainly one unique crystal form could be grown from a wide range of conditions e.g., large PEGs with and without the addition of 0.1-0.5 M of common salts and a pH around 7. The crystals with the best initial diffraction were grown from a drop that contained 0.1 mM HEPES pH 7.5, 10%, PEG 8000, 0.15 M LiC1, 10% ethylene glycol.

Diffraction quality crystals were grown from a 2 μl+2 μl hanging drop that contained 8% PEG 8000, 0.2M LiCl and 20% ethylene glycol. Due to the high concentration of ethylene glycol, the crystals could be harvested and flash frozen directly from the drops. Screening for anisotropically diffracting, non-twinned crystals was carried out using a Rigaku FRE rotation anode generator equipped with a mar225CCD detector and MSC X-stream cryo head and also at the beamline 911:2 at MaxLab equipped with a mar165CCD detector and X-stream cryo head. Crystals that showed ordered diffraction were stored in preparation for data collection on the ID-23 and ID-29 beamlines at the European Synchrotron Radiation Facility in Grenoble.

Diffraction data sets were collected from single crystals cooled at 100K at ESRF. The first data set was collected at beamline ID23, employing a ADSC Quantum Q315r detector. The data set was recorded at a wavelength of 1.072 Å over an angular range of 180° in rotation frames of 0.3°. The crystal diffracted to a nominal resolution of 3.2 Å and belongs to the monoclinic space group P21 with a cell dimensions of a=90.1 Å, b=89.7 Å, and c=250.8 Å with β=99.8°. This data set was used to solve the initial structure. A second data set to higher resolution, used for the final refinement, was collected at beamline ID29, employing a ADSC Quantum Q315r detector. This data set was recorded at a wavelength of 0.976 Å over an angular range of 220° in rotation frames of 0.2°. The crystal diffracted to 2.9 Å and belonged to the same monoclinic space group P21 with cell dimensions of a=90.2 Å, b=89.5 Å, and c=249.7 Å with β=99.8°. Reflections from each dataset were integrated with MOSFLM (Leslie, A. 1991 In Moras, D., Podjarny, A. D. and Thierry, J. C. (eds), Crystallographic Computing V. Oxford University Press, Oxford, UK, 27-38) and merged and scaled individually with the SCALA programs from the CCP4 suite (CCP4 (Collaborative Computational Project, Number 4) 1994 Acta Cryst. D 50, 760-763). Data collection and refinement statistics are shown in Table 11.

The crystallographic asymmetric unit contains four molecules of the Fab fragment and four molecules of the PAI-1 molecule resulting in a Matthews coefficient of 2.68 Å3/Da (Matthews, J. Mol. Biol. 33, 491-497) corresponding to a solvent content of 54%. The structure of the PAI-1 stability mutant/CAZ1001 Fab complex was determined by the method of molecular replacement using the CCP4 program MOLREP (CCP4 (Collaborative Computational Project, Number 4) 1994 Acta Cryst. D 50, 760-763). The structure was determined by using a crystal structure of PAI-1 stability mutant (pdb 1DVM (Stout, T. J. et al., 2000 Biochemistry, 39, 8460-8469)) and a crystal structure of Fab (PDB entry 1AQK (Faber et al., 1998 Immunotechnology, 3, 253-270)) as search models. Independent searches were done with PAI-1 and the constant and variable domains of the Fab.

Manual rebuilding of the Fab model and the introduction of the correct amino acid sequence were performed using the model building and validation program COOT (Emsley, P. & Cowtan, K. 2004 Acta Cryst. D60, 2126-2132). Loops that in the initial map were identified as being different to the search model, including most of the CDRs, were truncated and the remainder of the model was sent through crystallographic refinement using Autobuster (Roversi, P., et al., 2000 Acta Cryst., D56, 1313-1323).

A complete model could be built of the complex with the interpretation of the density of the CDR region facilitated by the access to the observation from the all the four independently determined complexes. The structure—containing 4 Fab molecules and 4 PAI-1 molecules was refined to convergence using Autobuster and in the remaining cycles REFMAC (Murshudov, G. N., et al., 1997 Acta Cryst., D53, 240-255) that was primarily used to tighten the stereochemistry and add 100 of the most well ordered water molecules. The final R-factors, Rwork and Rfree are 25.0% and 30.0%, respectively.

H. Generation of Human PAI-1 and PAI-1 Mutant Proteins for Epitope Mapping and Cloning of Human PAI-1 cDNA

The sequence of human PAI-1 was obtained from Embl (Accession No: M16006). Using this sequence oligonucleotide primers were designed to amplify the cDNA encoding human PAI-1 without the signal peptide. The N-terminal primer was HPAI1Nde and HPAI1XhoI was used as the C terminal primer (See Table 15 for oligonucleotides sequences).

A PCR reaction to amplify the cDNA was carried out. The template for the PCR reaction was 10 ng of cDNA obtained from human small intestine. The amplified cDNA from each reaction was purified and cloned into pCR4blunt topo (Invitrogen) using the topoisomerase ligation reaction according to the manufacturer.

Positive clones were identified and sequenced. The resulting cDNAs were sub-cloned using standard techniques into an E. coli T7-promoter expression vector in such a way that the cDNA encoding mature human PAI-1 was fused at the N-terminus with an N-terminal tag containing a HIS6 sequence immediately upstream of the sequence VHH of mature PAI-1 to generate the plasmid pT7H14hPAI1.

### I. Generation of the PAI-1 Stable Mutant

A stable mutant of human PAI-1 (Berkanpas, M. B., et al., 1995, supra) (SEQ ID NO: 190) with N150H, K154T, Q319L and M354I mutations (positions 171, 175, 340 and 375 respectively in SEQ ID NO: 190) was generated by site directed mutagenesis using a Quickchange™ multi mutagenesis kit from Stratagene according to the manufacturer's protocol. Mutagenesis primer design was performed according to the manufacturer's protocol and primers are shown in Table 15. Plasmid pT7H14hPAI1 was used in the single reaction as template. This was followed by subsequent DpnI digestion and transformation into chemically competent E. coli cells with selection on agar plates containing appropriate antibiotics at 37° C. overnight. Several clones were sequenced and one was identified which had all four of the mutations incorporated. This was designated plasmid pT7H14hstablePAI1.

### J. Generation of Mutations of the Stable PAI-1 Mutant for Epitope Mapping

Several further mutants of stable human PAI-1 were designed using information from the latent and active structures of human PAI-1. Oligonucleotides (Table 15) were designed in order to generate the mutants by site directed mutagenesis using the Quickchange™ multi mutagenesis kit from Stratagene. The template for each individual reaction was pT7H14hstablePAI1 and each individual mutagenesis reaction was carried out according to the manufacturer's protocol. Several clones were sequenced from each reaction and plasmid DNA of one correct clone from each reaction was retained for further use.

### K. Expression of PAI-1 Mutant Proteins

The HIS-tagged PAI-1 expression plasmids were transformed into chemically competent BL21 (DE3) star cells (Invitrogen) using the manufacturer's method. Transformed cells were used to inoculate 1 L cultures of Terrific Broth and these were incubated on an orbital incubator at 37° C., until the A600 reached 0.6. IPTG was then added to 0.25 mM and incubation continued for 4 h at 37° C. The cells were harvested by centrifugation and the cell pellets were stored at −80° C.

### L. Purification of PAI-1 Mutant Proteins

The cell pellets from 500 ml were thawed and resuspended in 50 ml Buffer A (50 mM HEPES pH7, 150 mM NaCl and 10 mM Imidazole) then lysed by sonication (4×30 seconds on ice at setting 6.5). The lysate was then spun at 100 000 g in Ti45 rotor for 30 minutes at 4° C. to give clarified lysate.

The lysate was loaded onto a 5 ml Ni-NTA column pre-equilibrated with buffer A. The sample was loaded onto the column and washed with 5 column volumes (CV) of buffer A followed by 5CV of 5% Buffer B (50 mM HEPES pH7, 150 mM NaCl and 300 mM Imidazole) and then 5 CV of 10% Buffer B. The protein was eluted with 100% buffer B. The fractions containing the PAI-1 protein were frozen in liquid nitrogen and stored at −80° C.

The frozen protein was thawed and was centrifuged at 45,000 rpm, 4° C. for 10 min. The protein was then concentrated at 4° C. using a Vivaspin 10 centrifuged at 4000 rpm. The concentrated protein was then run through a Superdex 200 1660 column equilibrated in 10 mM NaAc, 150 mM NaCl, 1 mM EDTA, pH5.6.

Fractions containing PAI-1 protein were pooled. The purity was checked by SDS PAGE gel electrophoresis, and a sample tested for PAI-1 activity using the tPA assay. 1 μg PAI-1 (7.5 μL) was mixed with 2.5 μL PBS and 2.5 μL 0.2 mg/mL tPA. A control of no tPA and no PAI-1 was also used. After heating at 37° C. for 10 min, the reaction was stopped by the addition of 12.5 μL×4 SDS PAGE loading buffer with no reducing agent. The samples were then run on an SDS-PAGE gel and stained. tPA forms a covalent complex with the active PAI-1 which runs at a higher molecular weight than the tPA only control.

Mass spectroscopy was used to measure the mass of each of the proteins. All of the proteins were of the predicted mass without the N-terminal methionine. Each purified PAI-1 protein was frozen in liquid nitrogen and stored at −80° C.

### M. BIAcore Measurements

BIAcore studies in Example 7, were undertaken using a BIAcore 2000™. Antibody 08 was coupled to the surface of a CM-5 sensorchip using an amine coupling kit to provide a surface density of 220-225 Ru. Stable mutant human PAI-1 or PAI-1 mutant proteins at a range of concentrations between 200 nM and 0.2 nM in HBS-EP buffer were passed over the sensor chip surface. The resulting sensorgrams were evaluated using BIA evaluation 3.1 software to calculate the kₒₙ, k_{off} and K_{D} values for the antibody tested.

### Example 14

### Efficacy of an Anti-PAI-1 Monoclonal Antibody in a Murine Model of Lupus Nephritis

MEDI-579, a monoclonal antibody targeting PAI-1 binding to uPA and tPA, was evaluated in a murine model of lupus nephritis using adenoviral delivery of interferon-alpha in NZBxNZW/F1 mice. Characterization of the model established that PAI-1 is increased (mRNA in the kidney and protein in both the kidney and plasma) following adenoviral delivery of interferon-alpha in this genetically predisposed mouse strain. This establishes a model for the evaluation of PAI-1 blockade in autoimmune-driven fibrosis.

The first efficacy study evaluated the fully human IgG1, MEDI-579, at 10 mg/kg delivered intraperitoneal (i.p.) twice per week for the study duration. This study demonstrated that MEDI-579 treatment decreased proteinuria (using a semi-quantitative measure via dipstick) and improved kidney pathology scores (H&E) but the changes were not statistically significant. Plasma active PAI-1 levels were found to be increased in diseased animals compared to naive or those administered empty vector and this increase was reduced with MEDI-579 treatment when measured at study week 4. However, upon study termination at week 6 this effect was lost and plasma levels of MEDI-579 were undetectable, indicating a possible immunogenicity response to the human antibody in the mouse model.

The subsequent efficacy study then evaluated a murinized version (e.g., a chimeric antibody) of the antibody which is composed of the same human CDRs on a murine Fc (herein referred to as muMEDI-579) at 1 and 10 mg/kg i.p. twice per week for the study duration. This study showed a dose-dependent and significant reduction in both proteinuria and kidney damage. Upon study termination at 5.5 weeks, active PAI-1 levels in both the plasma and kidney homogenates were found to be significantly reduced in the 10 mg/kg treatment group. Gene analysis of the kidneys revealed that muMEDI-579 treatment at 10 mg/kg normalized several genes related to coagulation, matrix turnover/fibrosis and inflammation.

The PK/PD study was performed in mice with measureable proteinuria (approximately week 4 post-adenoviral infection). Mice received a single dose of MEDI-579 at 10 mg/kg and terminal groups of mice were used to evaluate active PAI-1 in plasma and kidney homogenates at various time points following antibody administration. Following the administration of one dose of MEDI-579, there was a reduction in active PAI-1 with a concomitant elevation in plasmin and these effects were correlated with a high circulating level of MEDI-579 in the plasma. At one week following administration, MEDI-579 was no longer detectable in the plasma and these two biomarkers had returned to baseline.

Detailed findings of the present study are described below.

### PAI-1 Expression in Adv-Interferon-Alpha Accelerated Murine Model of Lupus Nephritis

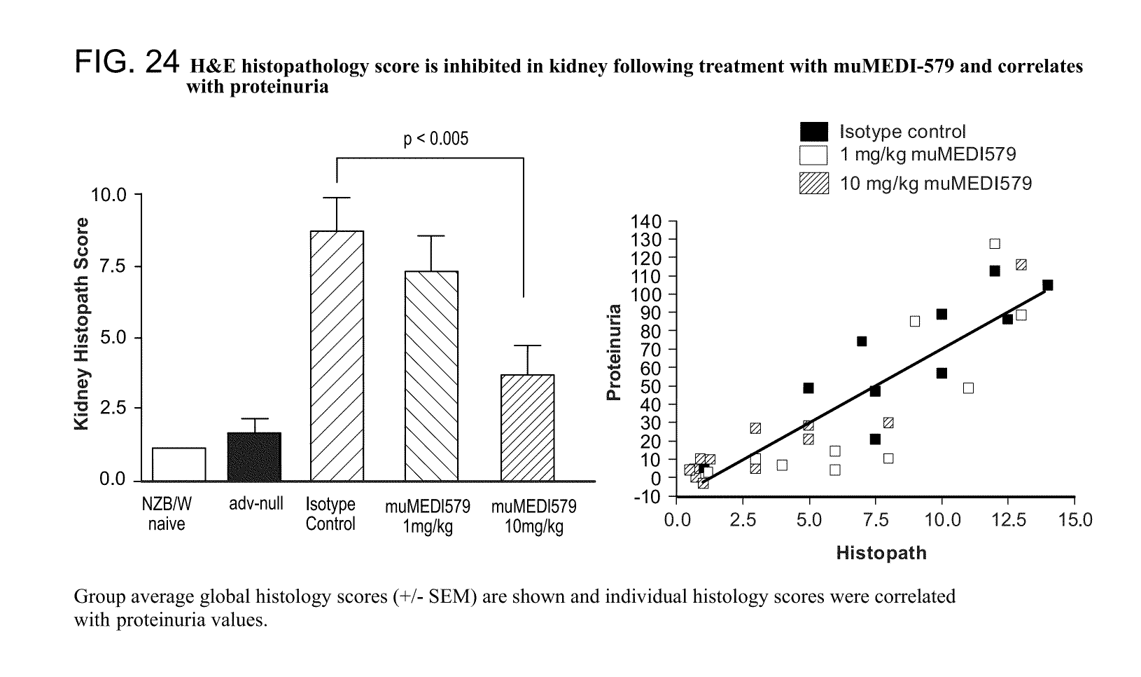

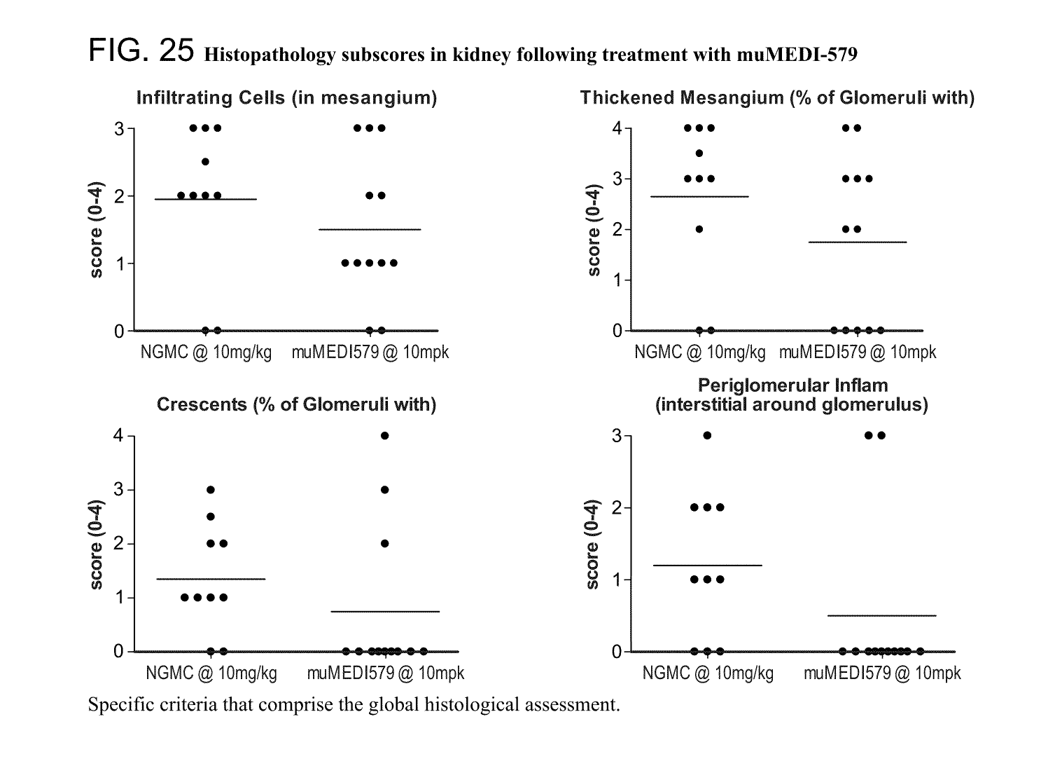

NZBW/F1 mice infected with Adv-interferon-alpha developed clinical signs of proteinuria (FIG. 24A), which corresponded with PAI-1 expression in the kidney. PAI-1 mRNA was detected in the kidneys of Adv-interferon-alpha infected mice and was found to be 2-3 fold higher than that of Ad Null infected mice at the same time points (FIG. 24B). Upon termination at 6 weeks, PAI-1 protein was detected in the glomeruli of kidneys from Adv-interferon-alpha, but not Ad Null, infected mice by immunohistochemistry (FIG. 24C). Active PAI-1 levels were found to be significantly increased in the plasma of Adv-interferon-alpha infected mice compared to the empty vector control mice (FIG. 25). Subsequent gene analysis also confirmed increased mRNA for PAI-1 (as well as other genes) following acceleration with Adv-interferon-alpha.

Initial Adv-Interferon-Alpha Accelerated Lupus Nephritis Study with MEDI-579

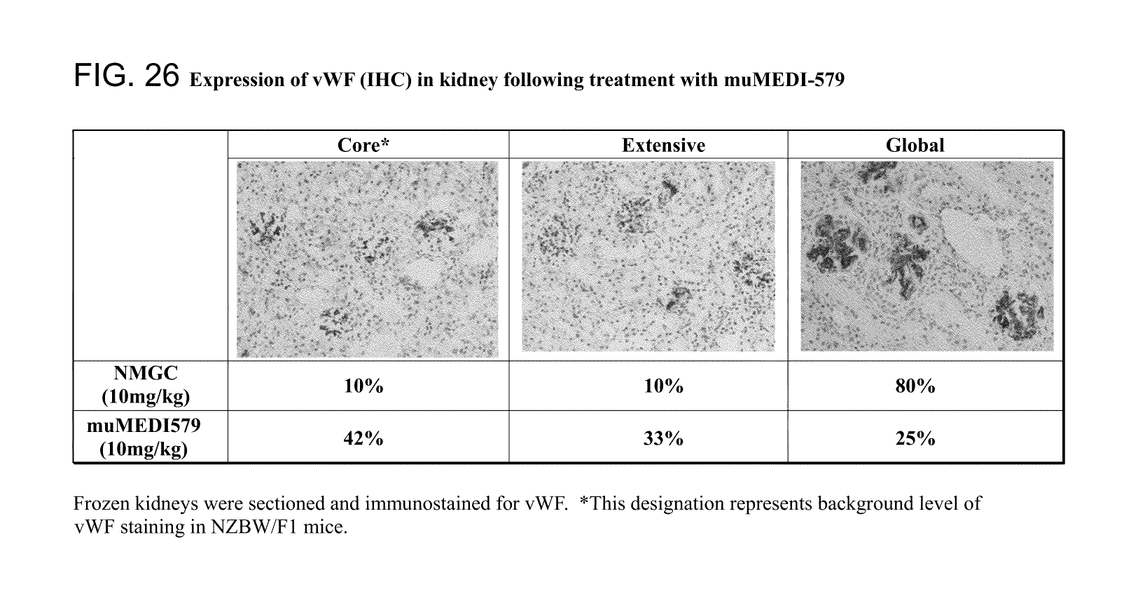

NZBW/F1 mice infected with Adv-interferon-alpha or Ad Null were administered an isotype control antibody, CAT-002, or MEDI-579 (10 mg/kg, twice weekly, i.p., starting day 0), proteinuria scores were assessed via the dipstick method throughout the course of this 6 week study. MEDI-579 at 10 mg/kg reduced the group average proteinuria scores compared to the CAT-002-treated mice (FIG. 26).

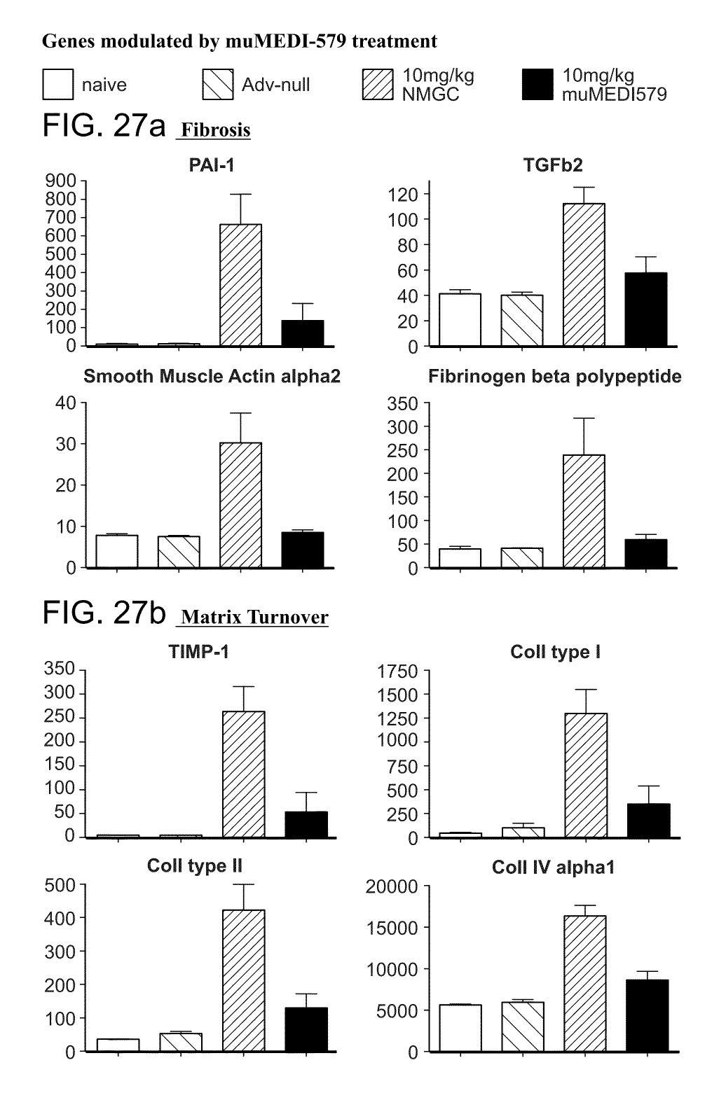

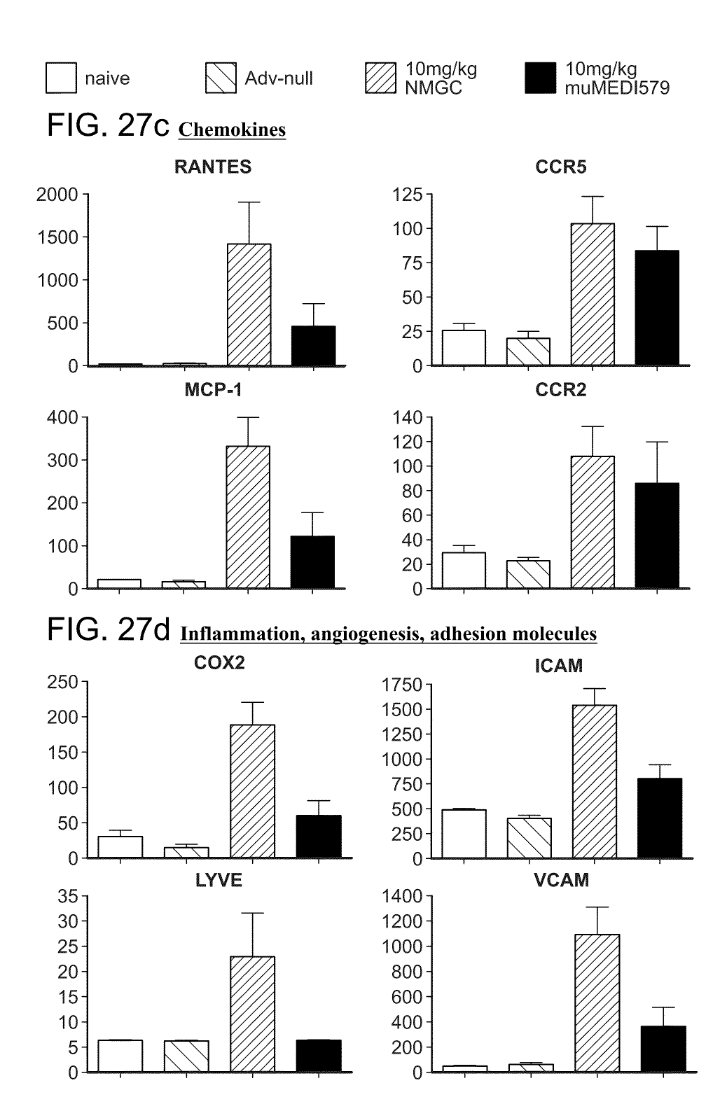

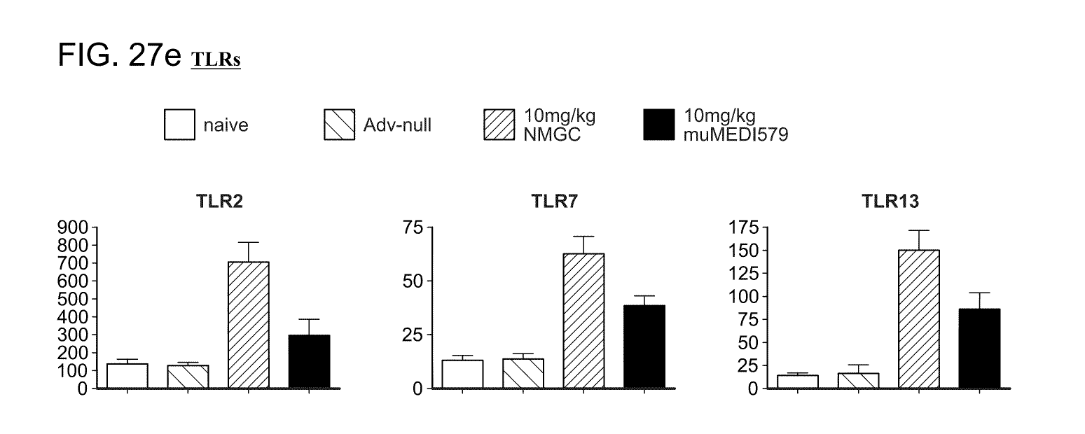

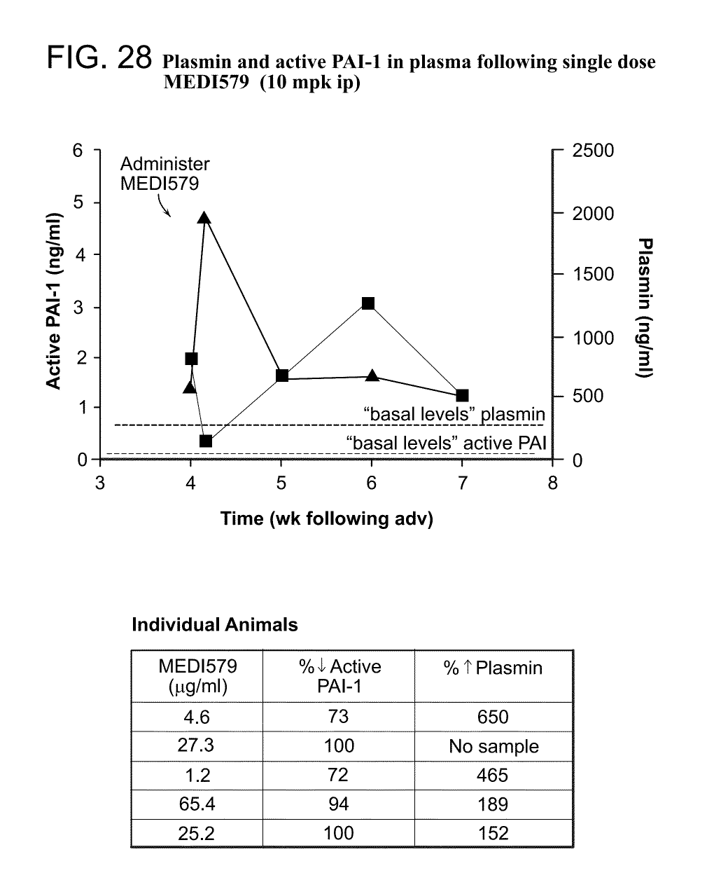

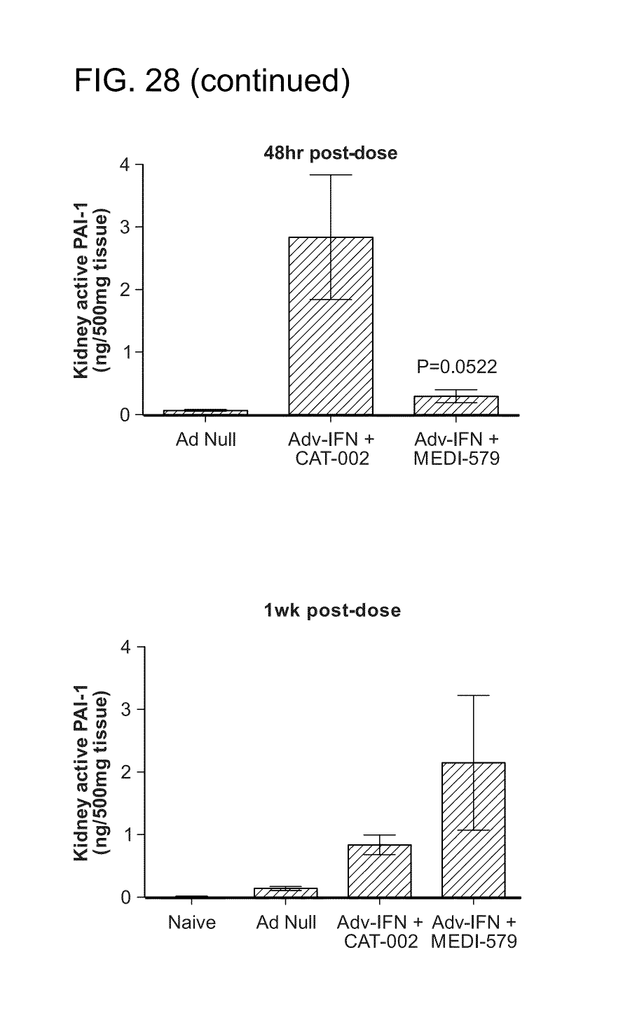

Active PAI-1 levels in the plasma of these mice was assessed at weeks 4 and 6 (FIG. 27). At week 4, MEDI-579-treatment reduced active PAI-1 levels in the plasma; however, this difference was not observed at week 6 and MEDI-579 levels were undetectable in the plasma, suggesting a loss of target coverage due to immunogenicity. Nevertheless, immunohistochemistry showed that MEDI-579 was bound within the glomeruli, which is consistent with the PAI-1 expression pattern within diseased kidneys (FIG. 28).

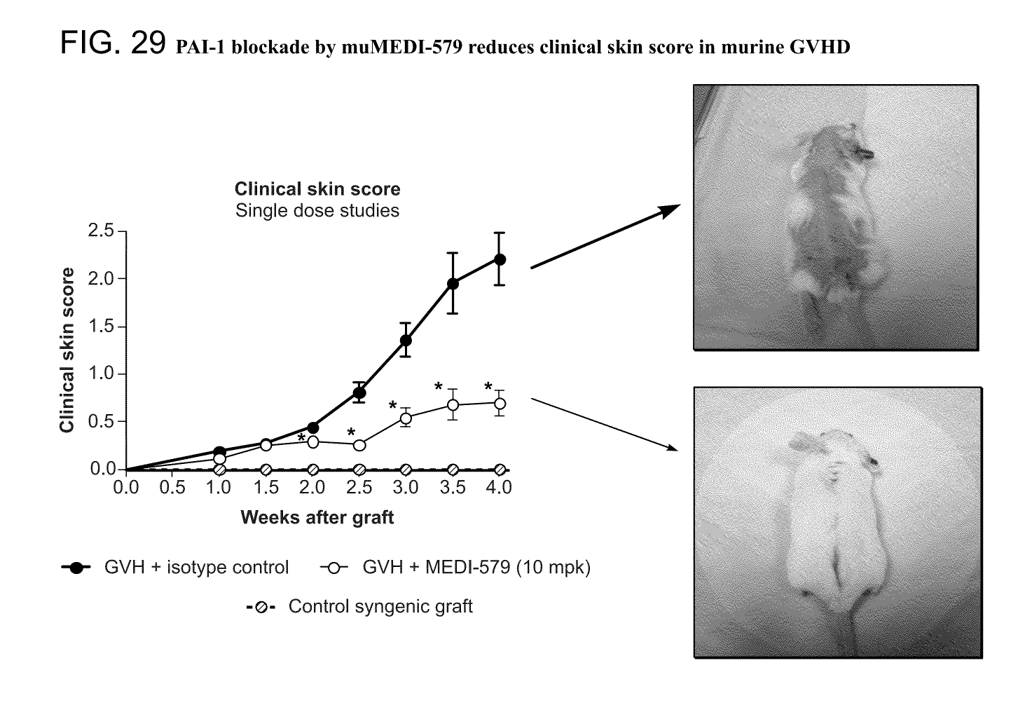

In order to determine whether efficacy was the result of an effect on the autoimmune drive known to occur in this model, FACS analysis of the splenocytes from these mice showed no difference in expansion of total lymphocytes, plasmacytoid dendritic cells, plasma B cells, marginal zone B cells or germinal center B cells between MEDI-579 and CAT-002 treated mice (FIG. 29), indicating that MEDI-579 is not working through alteration of immune cells.

Consistent with the proposed anti-fibrotic effect, histopathology following H&E staining performed on these kidneys suggested reduced glomerulosclerosis in the MEDI-579 treated group compared to the CAT-002 treated mice. Periodic acid-Schiff (PAS) staining suggested a decrease in glycoproteins within the mesangium with MEDI-579 treatment, summarized in the table below. The table shows a comparison of percent mice in the isotype (CAT-002) and MEDI579 treatment groups with either minimal/mild or moderate/severe glomerulosclerosis scores. Glomerulosclerosis is a target-relevant criteria that is one part of the global histology scoring system.

| | % Animals with | % Animals with |
|---|---|---|
| | Moderate/Severe | Minimal/Mild |
| Treatment | Glomerulosclerosis | Glomerulosclerosis |
| Isotype | 50% | 50% |
| MEDI579 | 10% | 90% |

Subsequent Adv-Interferon-Alpha Accelerated Lupus Nephritis Study with muMEDI-579

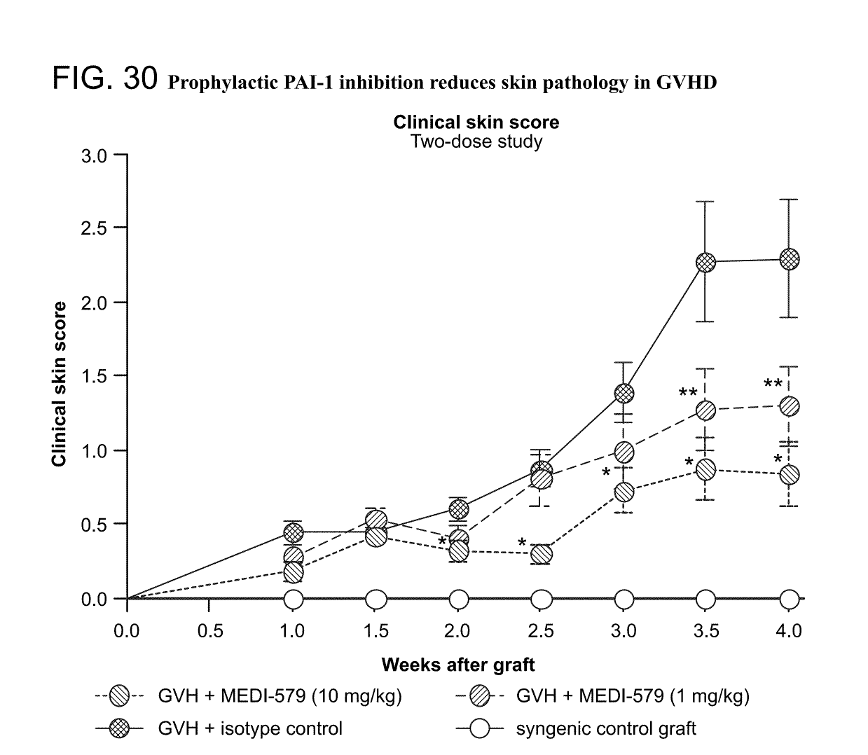

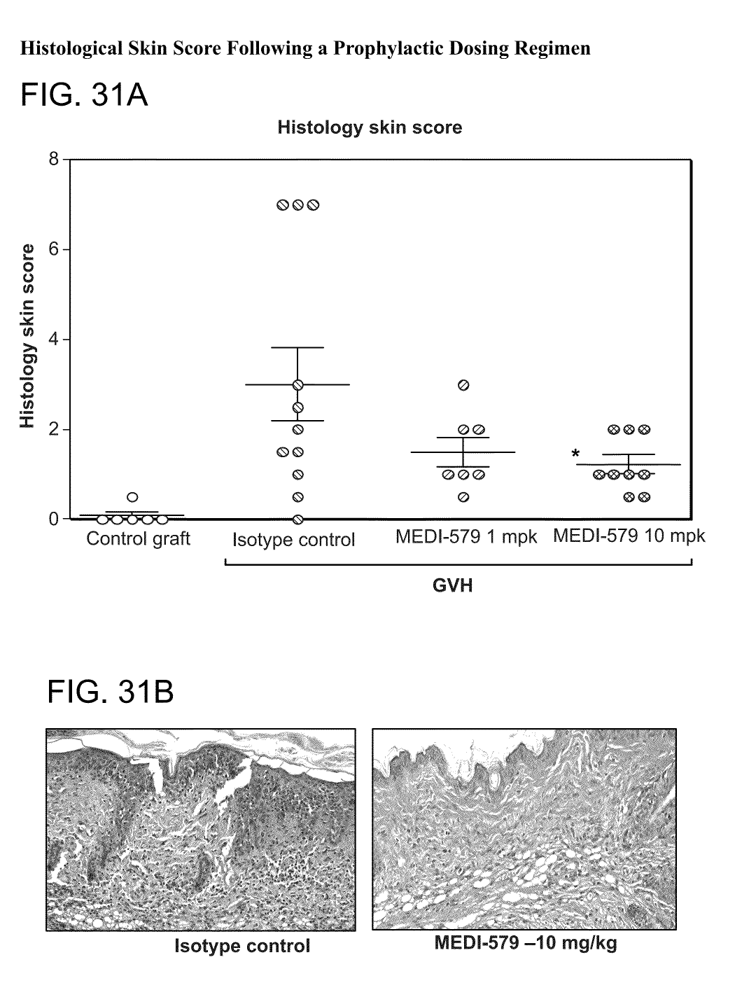

NZBW/F1 mice infected with Adv-interferon-alpha or Ad Null were administered either an isotype control antibody (NMGC) or muMEDI-579 (10 mg/kg, twice weekly, i.p., starting day 0). Proteinuria was assessed via quantitative analysis of 24 hour urine samples collected over three time points throughout the study. Treatment with muMEDI-579 resulted in dose dependent protection from the development of proteinuria (FIG. 30A) and protection from decreased urinary sodium output (another measure of renal function, FIG. 30B). This dose-dependent protection correlated with decreased active PAI-1 levels in the plasma and decreased total and active PAI-1 levels in kidney homogenates (FIG. 31).

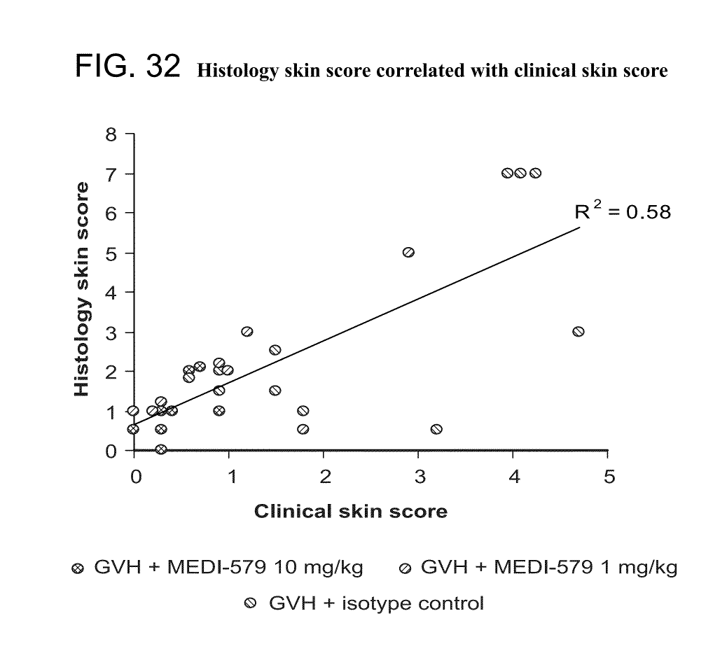

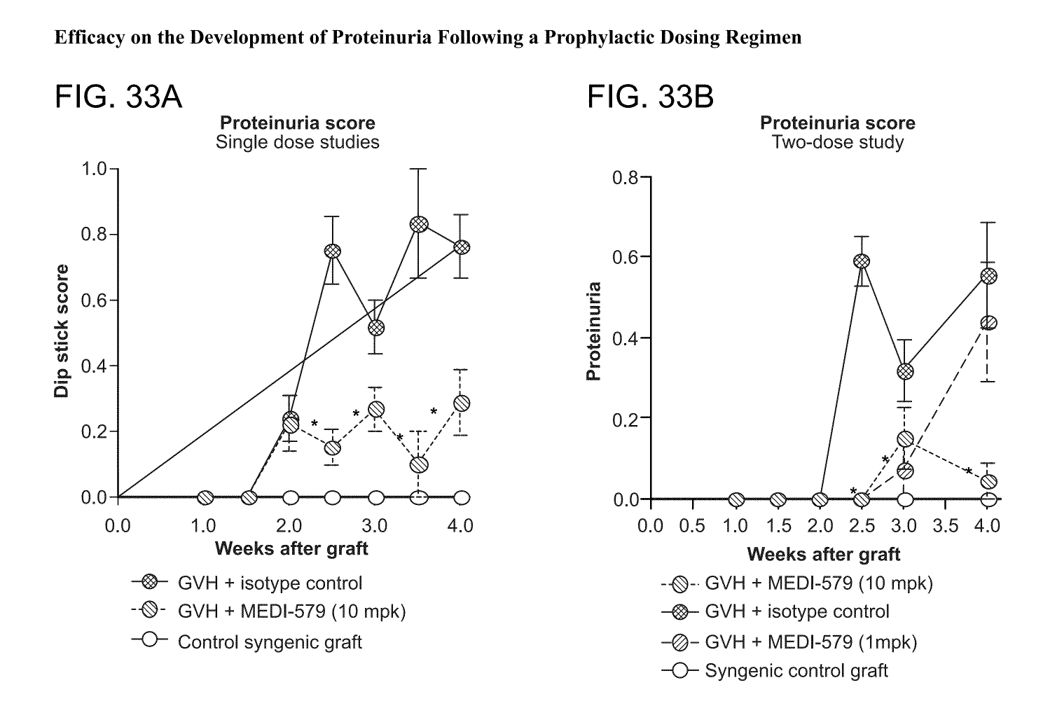

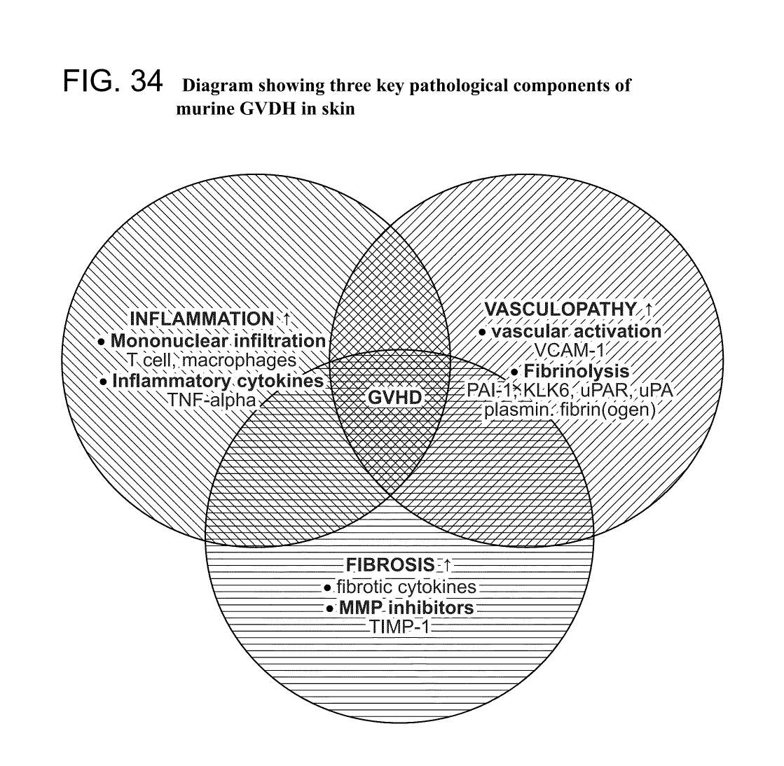

The global histopathology scores from the kidneys were significantly reduced in 10 mg/kg muMEDI-579 treatment group and a good correlation between histopathology and proteinuria was observed (FIG. 32). There appeared to be a trend for 10 mg/kg muMEDI-579-treated group to have lower scores associated with specific and relevant histological changes in the kidney: infiltrating cells in the mesangium, percent glomeruli with thickened mesangium, percent glomeruli with crescent formation and periglomerular inflammation (FIG. 33). Crescent formation within the glomeruli is driven by leaky vasculature (allowing infiltration of inflammatory cells into the Bowman's capsule) and by participation of coagulation factors (particularly fibrin), tissue factor and several different proliferating cell types including macrophages, parietal glomerular epithelial cells and interstitial fibroblasts. Periglomerular inflammatory cell infiltrate results from leaks in the Bowman's capsule. Immunohistochemistry for Von Willibrand Factor (vWF) was performed on these kidneys as a marker of endothelial damage/thrombosis. muMEDI-579 treatment resulted in a dose-dependent decrease of the extent and intensity of glomerular vWF staining (FIG. 34).

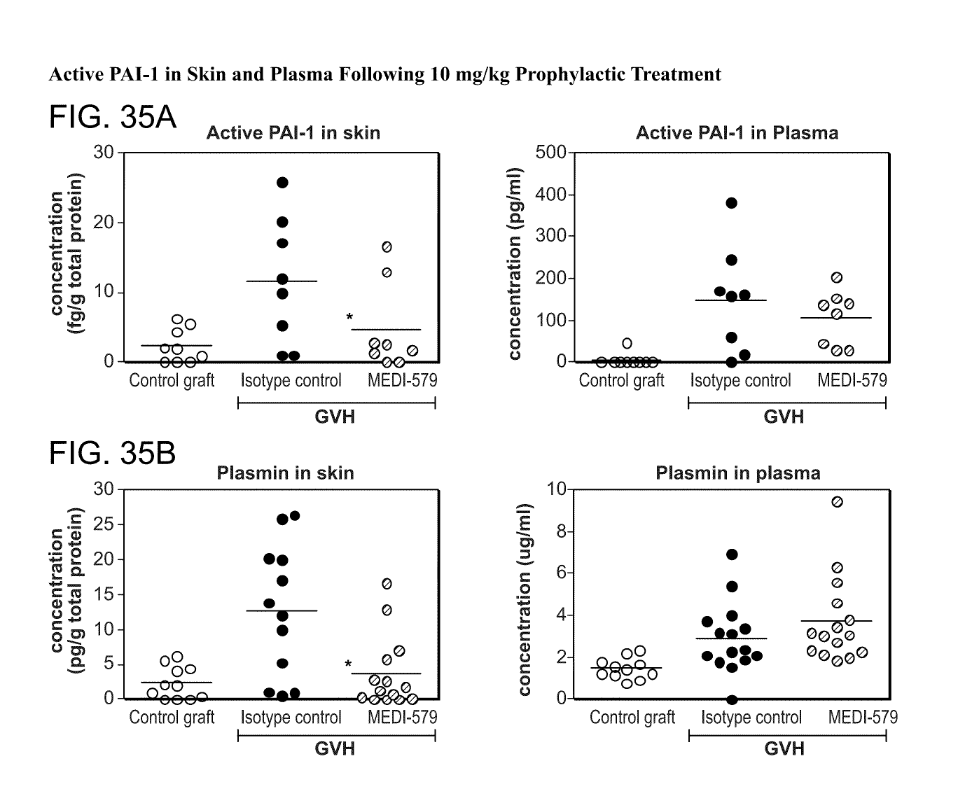

Gene analysis showed that treatment with 10 mg/kg muMEDI-579 normalized relevant genes within the kidneys (FIG. 35). These include genes related to coagulation, matrix turnover/fibrosis (PAI-1/TGF-b/SMA/TIMP/Collagens) and inflammation (chemokines/adhesion molecules/TLRs).

The overall results of muMEDI-579 treatment in this model is briefly summarized in the table below. Proteinuria is normalized to creatinine, naïve or Ad Null protein per creatinine=5-7 mg/dL/24 hr.

| | | 1 mg/kg | 10 mg/kg |
|---|---|---|---|
| | Isotype | muMEDI579 | muMEDI579 |
| Mice without Proteinuria | 8% | 33% | 50% |
| Urinary Protein | 75 | 58 | 39 |
| (mg/dl/24 hr) | | | |
| Urinary Sodium | ↓ | normal | normal |
| Kidney Histopath | ↑↑ | No effect | ↓ |
| Active PAI-1 | ↑↑ | ↓ | ↓↓ |
| Gene Changes | ↑↑ | N/D | ↓ |
| Mortality | 17% | 8% | 0% |

### A Single Dose of MEDI-579 Inhibits PAI-1 and Increases Plasmin in the Plasma of Accelerated Lupus Nephritis Mice

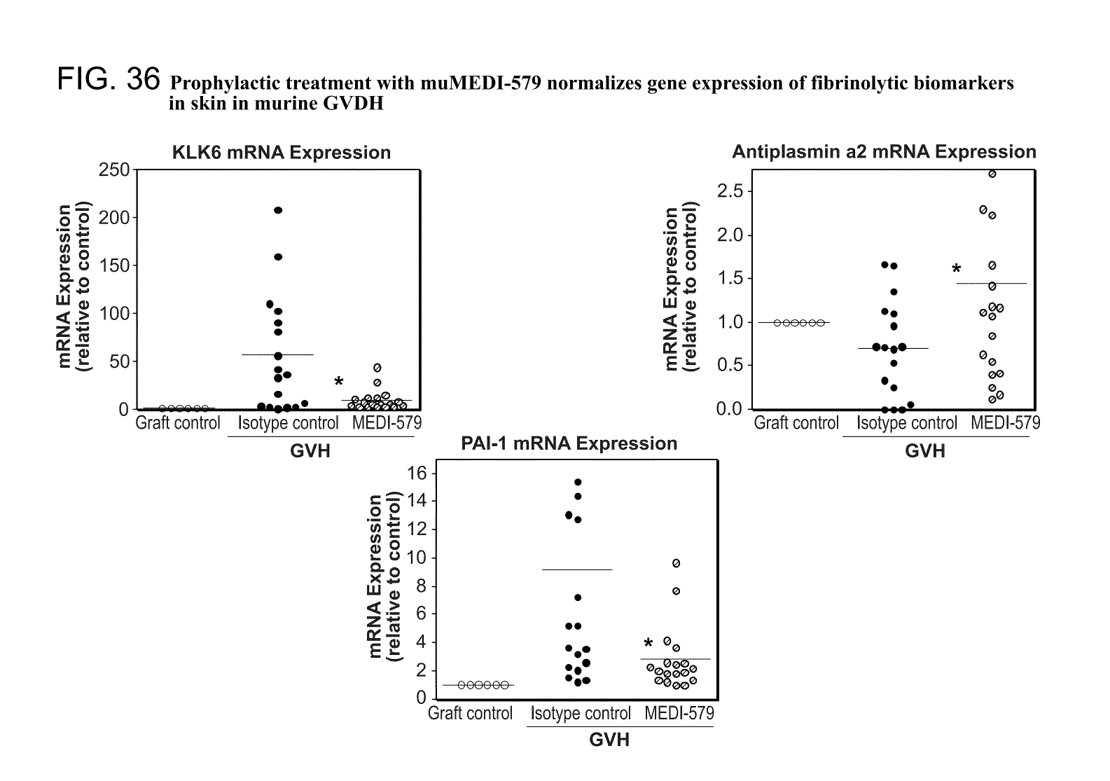

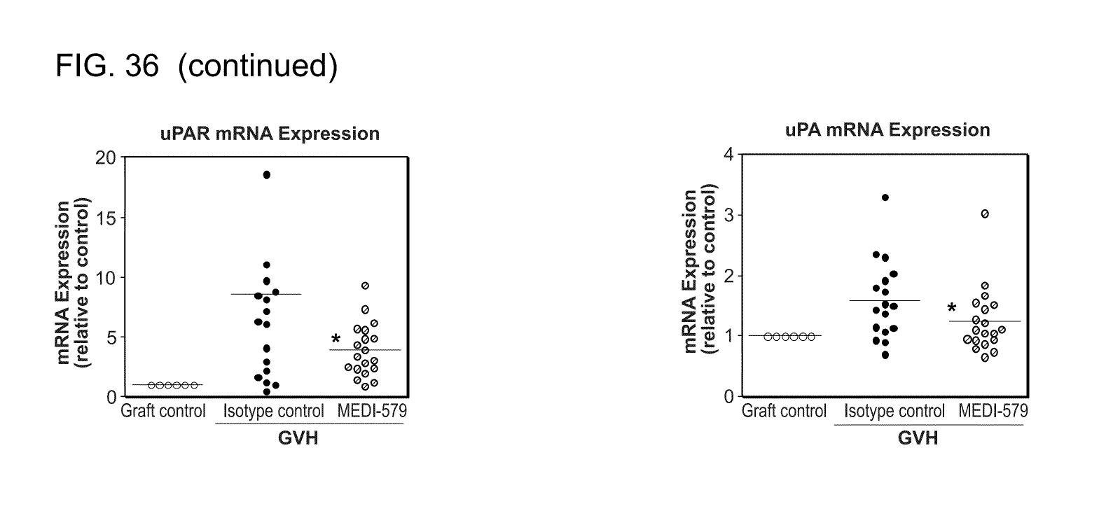

An exploratory PK/PD study was performed utilizing the accelerated lupus nephritis model. At a point in time following disease onset when active PAI-1 is elevated chronically (4 wk), animals were treated with a single administration of 10 mg/kg MEDI-579. Forty-eight hours following antibody administration, active PAI-1 was inhibited and plasmin levels increased in the plasma. These levels returned to baseline levels by the one week following administration (FIG. 36). Study design and methods for the present example are detailed below.

### Acceleration of Disease in Lupus Prone Mice

On day 0, mice were injected intravenously (lateral tail vein) with 0.3×10¹⁰ V.P. of either Ad Null vector or Adv-interferon-alpha in 0.1M phosphate buffered saline (PBS) pH 7.2 (Gibco).

### Test Articles

Twice weekly dosing began immediately following adenovirus delivery on day 0 and continued throughout the course of the study. Either CAT-002 (control, human IgG1), MEDI-579 (anti-PAI-1, human IgG1), NMGC (control, human IgG1) or muMEDI-579 (anti-PAI-1, MEDI-579 CDRs attached to a murine Fc, IgG2a) in PBS pH 7.2 (Gibco) were administered intraperitoneally at either 1 or 10 mg/kg of body weight, in a 0.2 ml volume.

### Assessment of Proteinuria

Urinary protein levels were roughly assessed (spot check) via urinalysis dipsticks (Chemstrip 2 GP, Roche) twice weekly, using the following scoring system; negative/trace=0, 30 mg/dL=1, 100 mg/dL=2, 500 mg/dL=3. Mice that died on study were assigned a score of 5 (weighted for mortality). Mice were placed in metabolic cages on a weekly basis in order to collect 24 hour urine samples, which were sent to AniLytics (Gaithersburg, Md.) for chemistry analysis.

### Assays for Biomarkers and Therapeutic Antibody Levels

Citrated plasma was collected by harvesting whole blood into Eppendorf tubes containing 3.2% sodium citrate ( 1/10 the total sample volume). The blood was then centrifuged, plasma was drawn off, put into a clean Eppendorf tube and centrifuged again, the samples were then frozen until assayed. Kidneys collected on the terminal day were homogenized in ice-cold neutral lysis buffer (1% Triton X-100, 1 mM EDTA and protease inhibitors in PBS pH 7.2). The lysates were centrifuged and supernatants were transferred to new tubes, then frozen until assayed. Active mouse PAI-1, active mouse plasmin and mouse fibrin(ogen) ELISA kits were purchased from Innovative Research (Nova, Mich.). Manufacturer instructions were followed for all assays. Frozen plasma samples were sent to MedImmune in Hayward, Calif. for determination of therapeutic antibody levels.

### Histopathology/Immunohistochemistry Processing and Scoring

Mice were terminated and left kidneys were harvested, halved and fixed in 10% neutral buffered formalin or fresh frozen in OCT tissue compound. Tissues were routinely processed, sectioned at approximately 4 um thickness, mounted on glass slides and stained with hematoxylin and eosin (H&E). Periodic-Acid Schiff (PAS) staining for proteoglycans was also performed on selected sections. Immunohistochemical labeling with rabbit anti-human Von Willebrand Factor (Abcam, Cambridge Mass.) and goat anti-rabbit IgG (Dako, Denmark) with a biotin conjugate were applied to selected treatment groups.

Kidneys were evaluated in a blinded study and scored on a scale defined as 0 (normal), 1 (minimal change), 2 (mild change), 3 (moderate change) to 4 (marked change). Serial sections were evaluated histologically and an average score appointed for each animal based on degree of inflammatory infiltrate, glomerular changes (hypercellularity, mesangial thickening, crescent formation, increased size, periglomerular thickening and presence of protein), proximal tubular and interstitial changes. Special stains were applied to selected sections, but scores were not included in the overall score. Immunohistochemical evaluation was performed and sections were scored based on overall intensity of labeling and distribution of signal.

### Total RNA Extraction and Microarray Processing/Analysis

Total RNA was extracted from kidney tissue using the Qiagen RNeasy Fibrous Tissue Mini kit (Hilden, Germany). RNA purity and concentration were determined spectrophotometrically (260/280>1.9). RNA quality was assessed on an Agilent 2100 Bioanalyzer using the RNA 6000 Nano LabChip®. The generation of biotin-labeled amplified cRNA from 2 mg of total RNA was accomplished with the Affymetrix GeneChip® One-Cycle cDNA Synthesis kit and the Affymetrix GeneChip® IVT Labeling kit. The concentration and purity of the cRNA product were determined spectrophotometrically. Twenty micrograms of each biotin-labeled cRNA was fragmented for hybridization on Affymetrix Human Genome U133 Plus 2.0 GeneChip® arrays. All GeneChip® washing, staining, and scanning procedures were performed with Affymetrix standard equipment. Data capture and initial array quality assessments were performed with the GeneChip Operating Software (GCOS) tool. Stratagene's (La Jolla, Calif.) ArrayAssist® Lite software was used to calculate probe-level summaries (GC-RMA) from the array CEL files.

### Example 15

### Efficacy of Mouse Surrogate Anti-PAI-1 Antibody muMEDI-579 in a Murine Graft-vs-Host Model of Scleroderma

Targeting PAI-1 in scleroderma provides a dual opportunity to impact the disease. Underlying the disease is a vasculopathy which leads to, and is exacerbated by, thrombosis. Inhibition of PAI-1 promotes fibrinolysis and resolution of thrombosis. Inhibition of PAI-1 should also contribute to the re-establishing matrix accumulation/degradation homeostasis by inhibiting fibrosis via promoting plasmin-associated matrix metalloproteases (MMPs) activation and fibrinolysis of the provisional fibrin matrix.

As previously described herein, muMEDI-579 (chimeric human Fab and mouse Fc monoclonal antibody; same CDRs as the fully human antibody MEDI-579) is capable of blocking PAI-1 binding to uPA/tPA. muMEDI-579 was administered prophylactically and therapeutically in mice induced with graft-versus-host disease (GVHD). GVHD is a model of scleroderma that recapitulates the three key pathological manifestations of the human disease in skin: inflammation, fibrosis and occlusive vasculopathy. Prophylactic PAI-1 blockade by muMEDI-579 conferred significant protection from clinical and histological manifestations in skin, as well as proteinuria, with an apparent dose response relationship. Efficacy of muMEDI-579 on skin scores was associated with normalization of fibrinolysis (plasmin, PAI-1-uPA, uPAR, KLK6, antiplasminalpha2) and vascular activation (VCAM-1) in skin. muMEDI-579 was efficacious in GVHD on a broad scale, as it prevented extensive skin inflammation (CD4, TNF-alpha, IL-33) and fibrosis (TIMP and CTGF). Reduction of hallmark T-cell adhesion molecules (VCAM-1) in dermal vascular tissues by muMEDI-579 correlated with reduction of T-cell infiltration in skin, suggesting that resolution of the vasculopathy component of GVHD by muMEDI-579 may drive reduction of inflammation in skin. Consistent with the known function of inflammation in the onset of fibrosis in GVHD, reduction of proinflammatory TNF-alpha in skin by muMEDI-579 correlated with reduction of a key TNF-alpha-mediated fibrotic factor (TIMP-1).

Similar to the prophylactic treatment regimen, muMEDI-579 administered following the onset of clinical symptoms conferred significant protection from clinical and histological progression, including fibrosis and normalization of fibrinolysis in skin. Detailed findings of the present study are described below.

### (I) PROPHYLACTIC STUDY.

PAI-1 Blockade by muMEDI-579 Reduces Clinical Skin Score in Murine GVHD

In-life clinical score was assessed on a biweekly basis until termination at week 4 post graft of single dose efficacy studies of muMEDI-579 in murine GVHD. Average of three independent experiments showed that muMEDI-579 at 10 mg/kg in a prophylactic setting significantly reduced clinical skin score as soon as two weeks post graft compared to isotype control (3.1-fold at week 4, p=0.0001), keeping clinical skin scores close to basal levels as represented by syngeneic control graft. Mice treated with isotype control mAb developed extended alopecia and skin lesion in back and front. In contrast, mice prophylactically treated with muMEDI-579 developed only restricted alopecia and skin lesion (FIG. 37).

Dose-dependent efficacy study of muMEDI-579 in murine GVHD showed a significant dose-response relationship between muMEDI-579 and skin score in prophylactic setting (FIG. 38). Although muMEDI-579 significantly improved skin score at both high and low doses compared to isotype control (10 mg/kg, p=0.0003; 1 mg/kg, p=0.0210), high dose achieved reduction of skin at a larger extent (2.75- vs. 1.76-fold at week 4) and faster (2 weeks vs. 3.5 weeks).

PAI-1 Blockade by muMEDI-579 Reduces Histology Skin Score in Murine GVHD

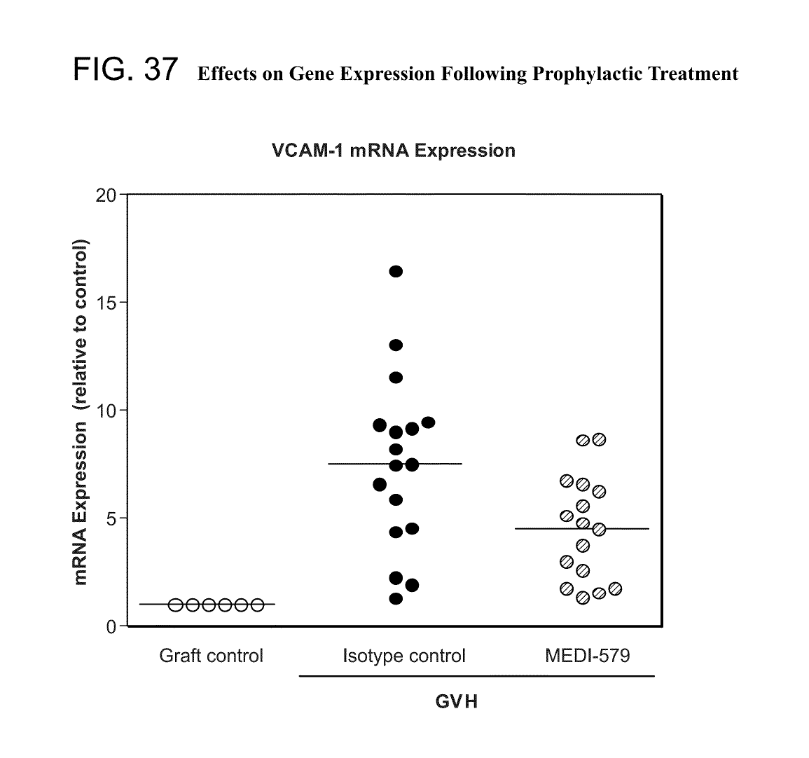

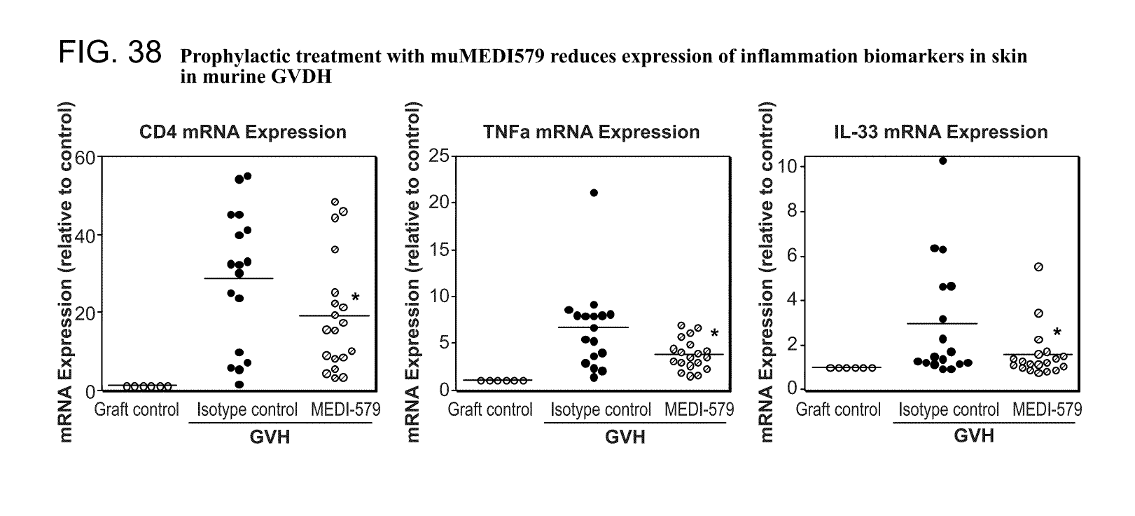

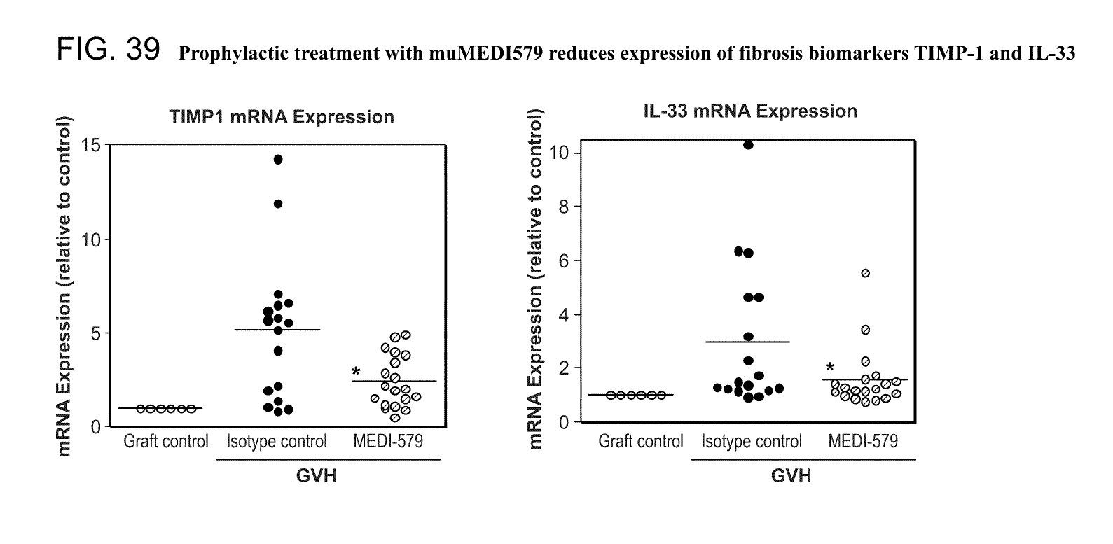

Histology skin score was assessed at 4 week post graft of a dose-dependent efficacy study in murine GVHD. muMEDI-579 at 10 mg/kg significantly reduced histology skin score compared to isotype control (FIG. 39A, p=0.0351). This reduction was based on lower lymphocyte infiltrates, lower epidermal hyperplasia and lower ulcer incidence in skin (FIG. 39B). The fibrosis component of the score was not assessable at termination—4 weeks, as fibrosis has yet to peak. muMEDI-579 at 1 mg/kg dose also reduced histology skin score close to significance levels compared to isotype control (p=0.0889).

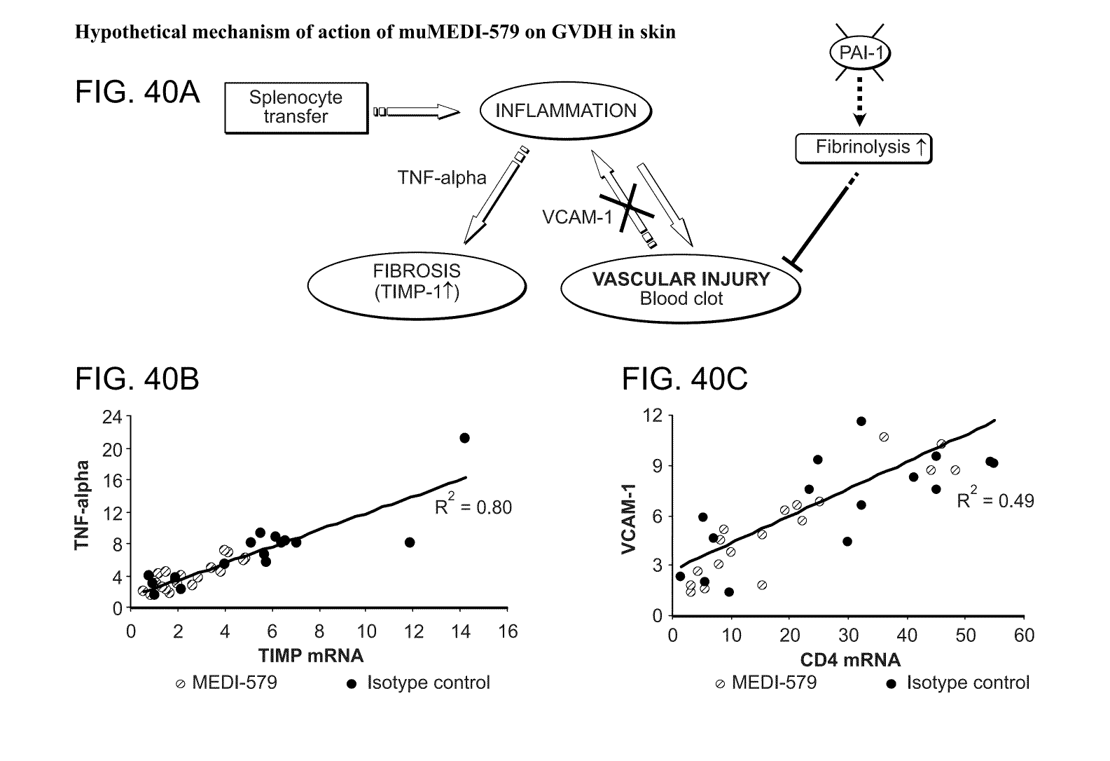

Of note, histology skin score correlated with clinical skin score, validating these scoring systems in assessing disease activity in murine GVHD (FIG. 40, R²=0.58).

PAI-1 Blockade by muMEDI-579 Reduces Proteinuria in Murine GVHD

In-life proteinuria was assessed by dipstick on a biweekly basis until termination at week 4 post graft of single dose efficacy studies of muMEDI-579 in murine GVHD (FIG. 41A). muMEDI-579 at 10 mg/kg in a prophylactic setting significantly reduced proteinuria as early as two-and-half weeks post graft compared to isotype control (2.7-fold at week 4, p=0.0008). Two-dose study showed that unlike at 10 mg/kg, muMEDI-579 at 1 mg/kg could not achieve significant reduction of proteinuria levels in a prophylactic setting in murine GVHD compared to isotype control (FIG. 41B, 4 week post graft, p=0.002 at 10 mg/kg; p=0.47 at 1 mg/kg).

muMEDI-579 Normalizes the Three Key Pathological Components of Murine GVHD

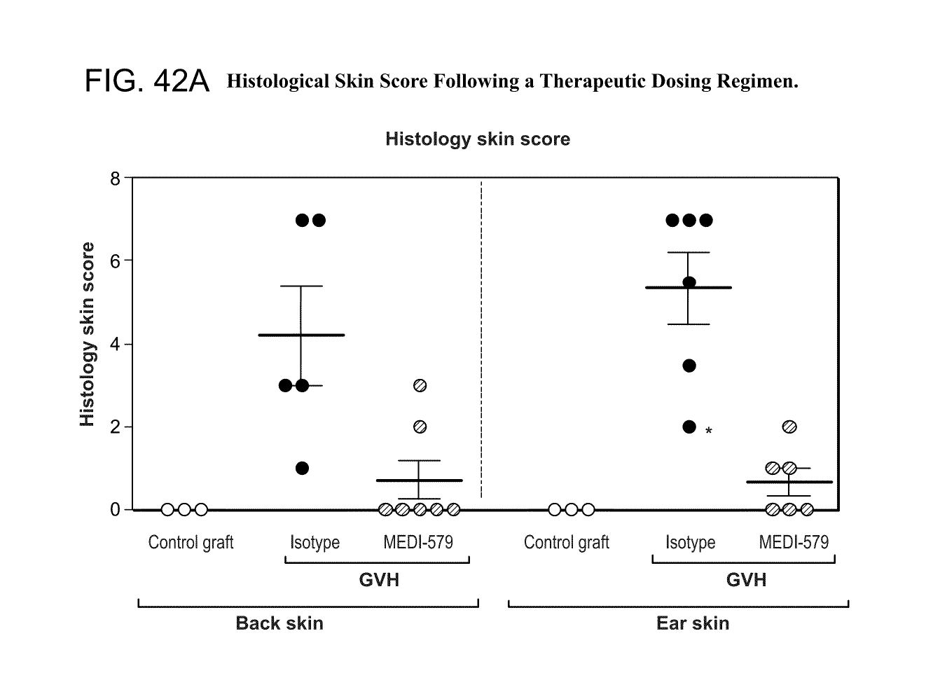

Similar to human scleroderma, murine GVHD is a systemic pathology with fibrotic, inflammatory and vascular components (FIG. 42). Inflammation and vasculopathy are early events (2-3 weeks) that precede late fibrosis (5-6 weeks) in various tissues, including skin and kidney. Profiling of GVHD gene-based biomarkers in skin showed that muMEDI-579 rescued mice from developing dermal GVHD on a broad scale. muMEDI-579 not only reduced the vasculopathy/fibrinolysis component of GVHD but also inflammation and fibrosis.

Efficacy of muMEDI-579 in Vasculopathy

### Fibrinolysis

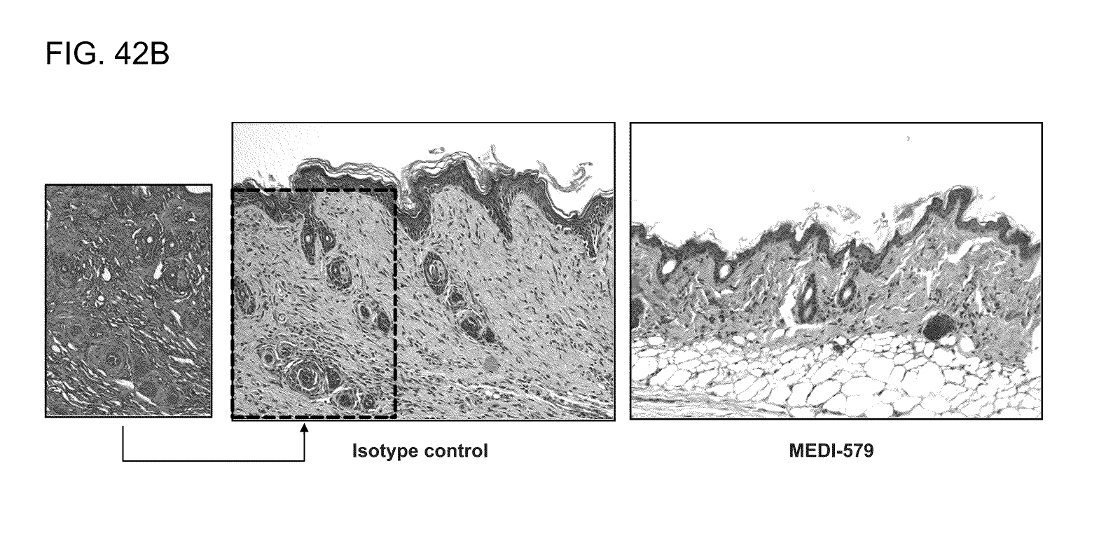

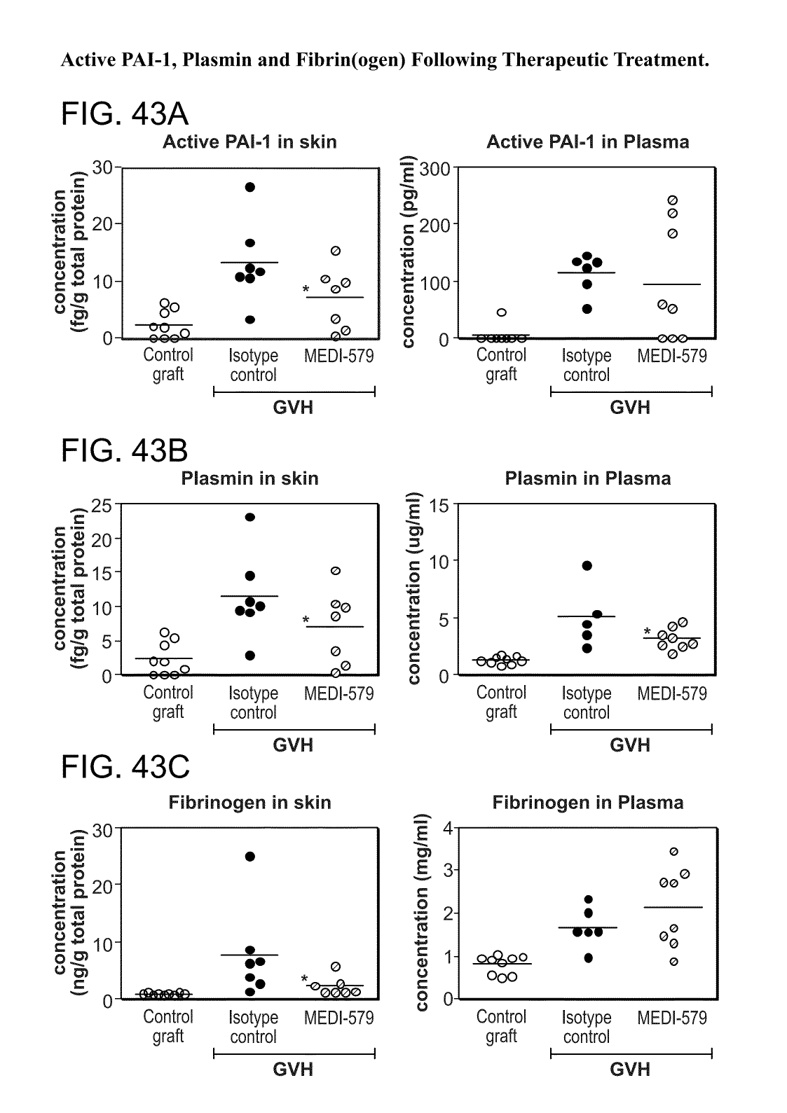

Prophylactic treatment with muMEDI-579 significantly reduced protein levels of free/active forms of PAI-1 and plasmin locally in skin in murine GVHD compared to isotype control at week 4 post graft. This reduction was not detected in systemic plasma compartment, possibly due to elapsed time following last dose (FIG. 43). While the fibrinolytic function of PAI-1 blockade is consistent with increase in plasmin in an acute setting (documented in accelerated NZB/W model), decrease in active plasmin in murine GVHD reflects a long-term, progressive beneficial impact of PAI-1 blockade on global GVHD pathology in this chronic, non acute model: muMEDI-579 normalizes fibrinolysis back to normal basal levels, as defined by control syngeneic graft.

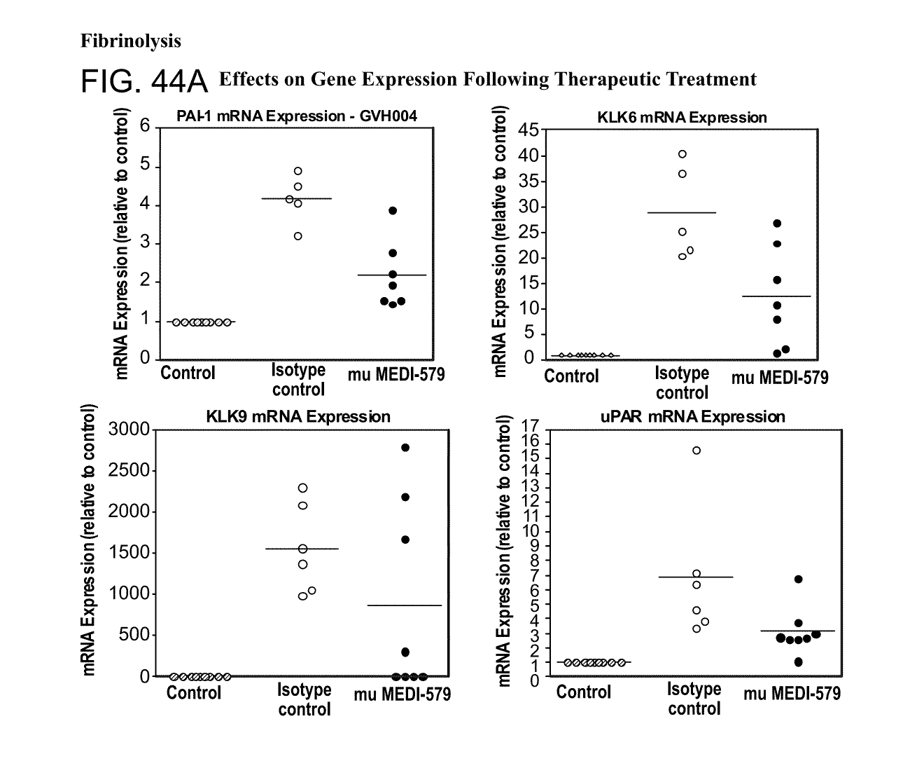

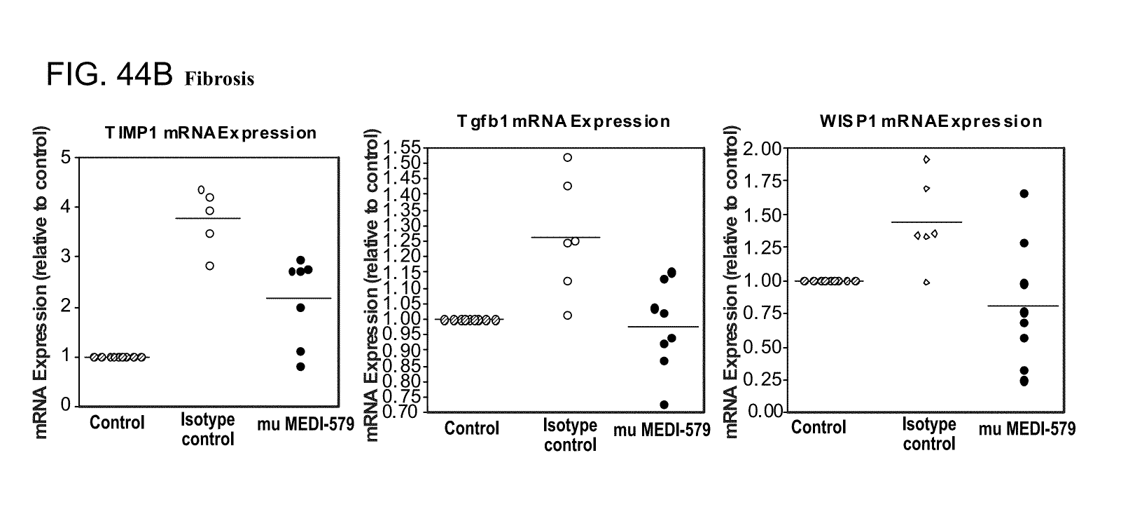

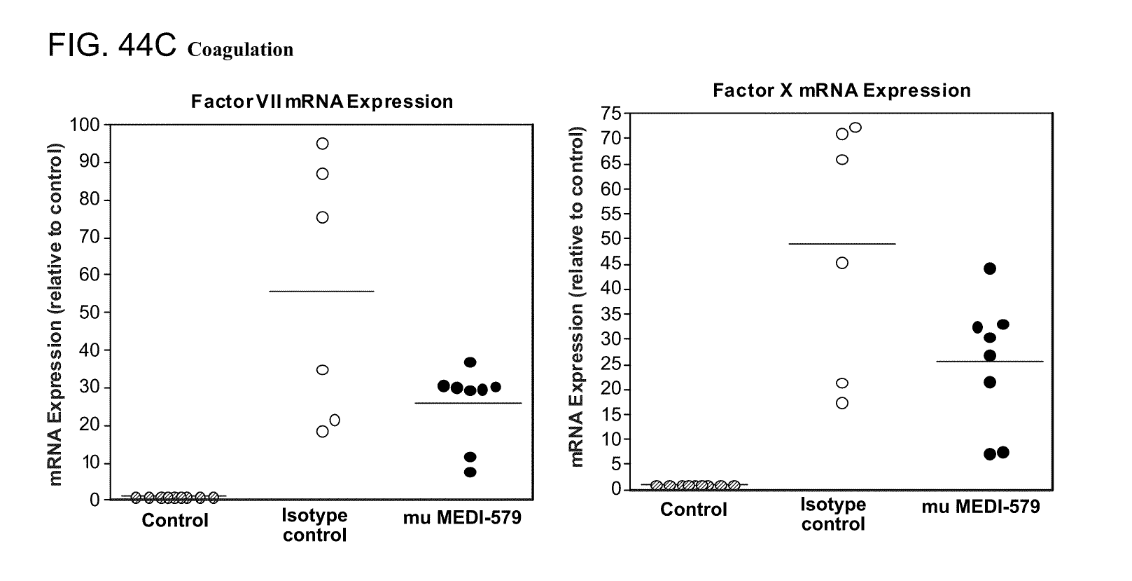

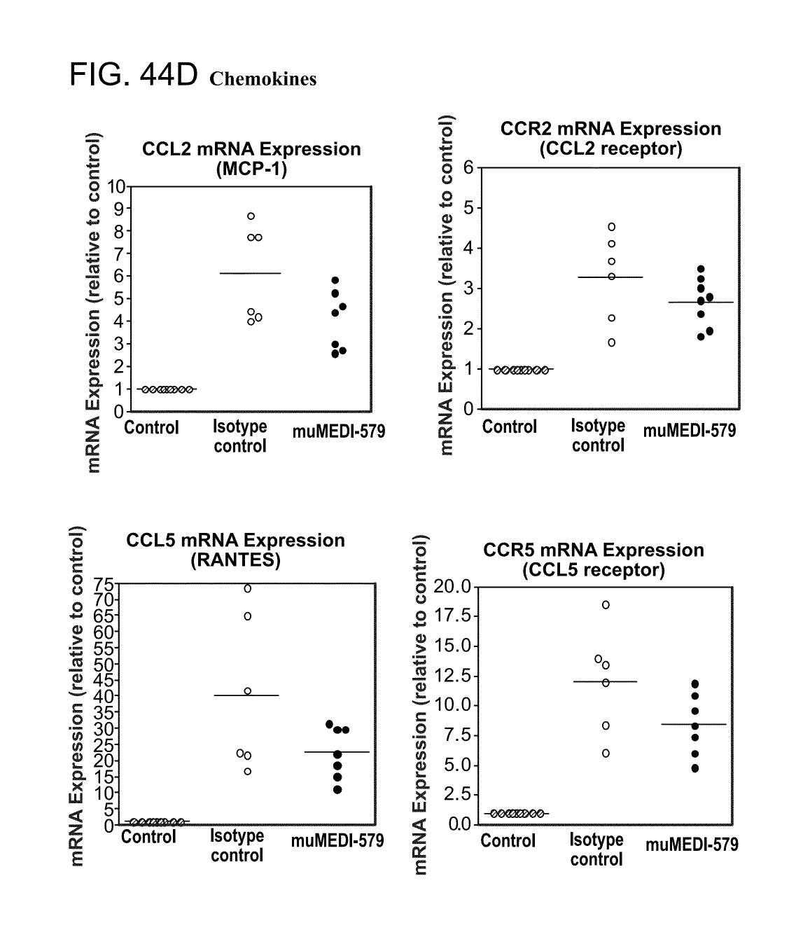

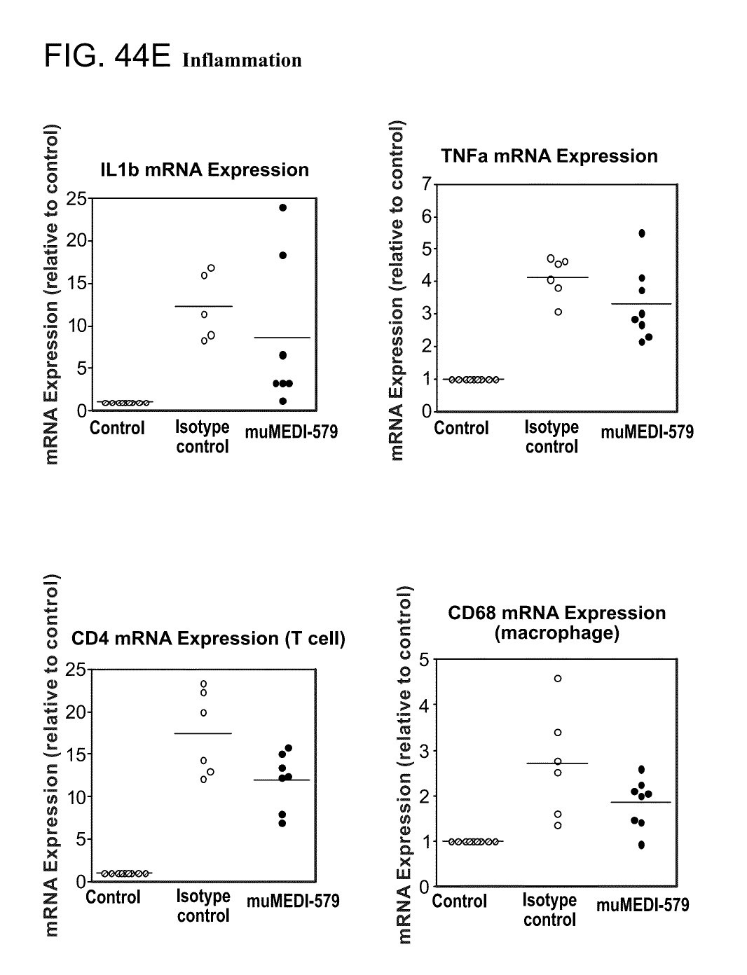

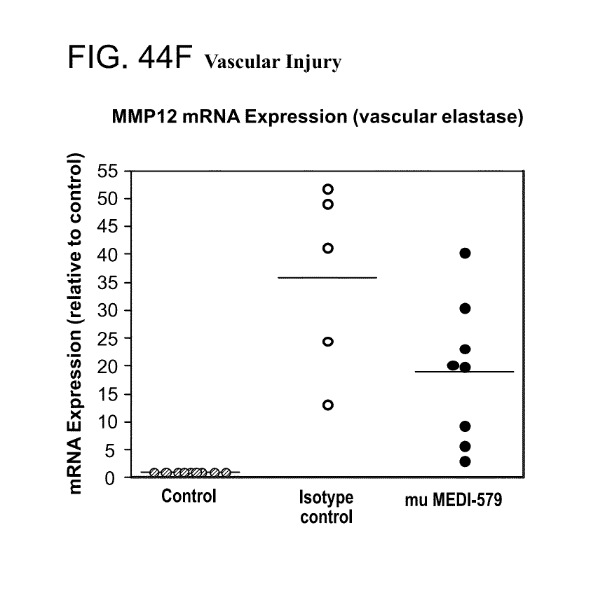

Gene profiling in skin revealed extended impact of muMEDI-579 on fibrinolysis, as muMEDI-579 reduced/normalized gene expression of KLK6, uPAR, uPA, and PAI-1 in skin (FIG. 44).

### Vascular Activation

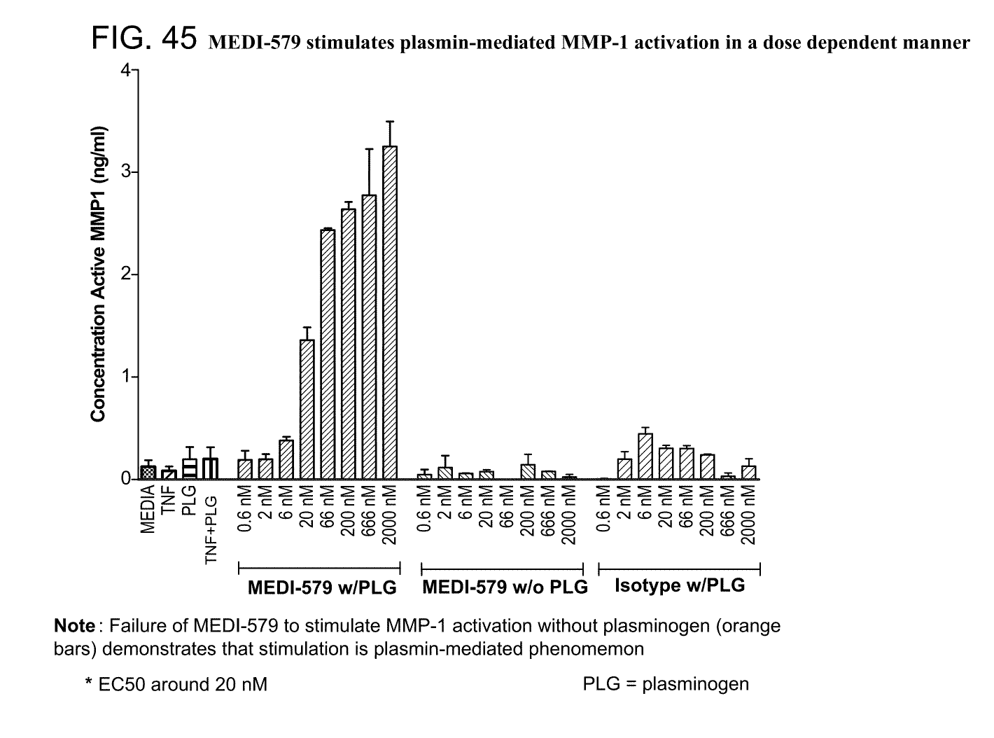

Prophylactic treatment with muMEDI-579 also significantly reduced gene expression of key vascular activation marker and lymphocyte-adhesion molecule, VCAM-1, in murine GVHD compared to isotype control at week 4 post graft (FIG. 45). This result suggests that muMEDI-579 has a beneficial effect on injury-induced vascular activation.

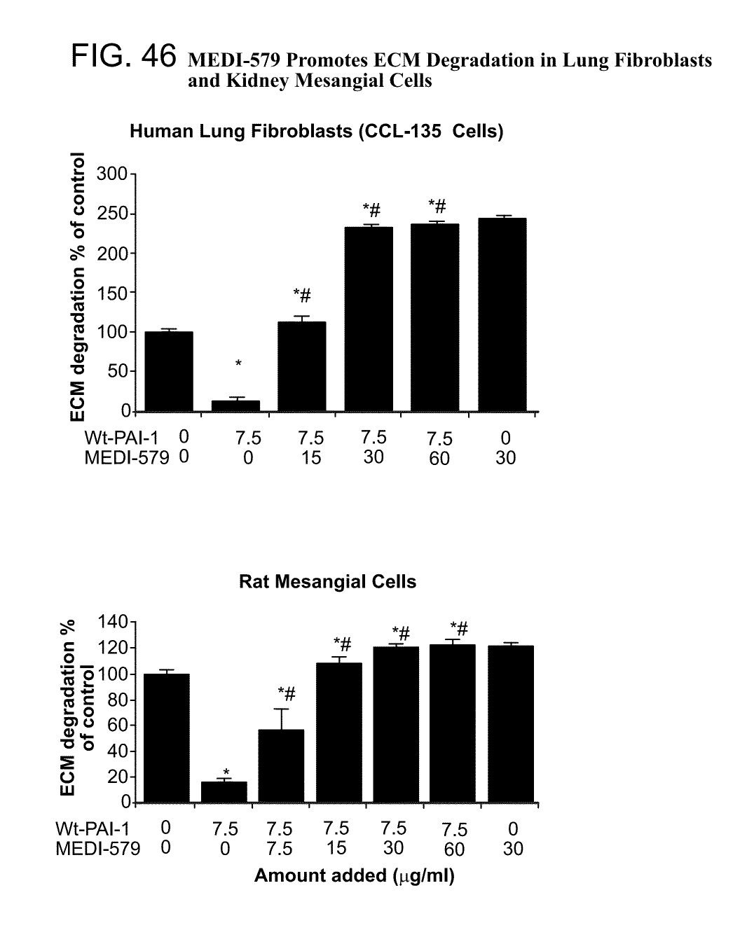

Prophylactic treatment with muMEDI-579 significantly reduced gene expression of key inflammation biomarkers in skin in murine GVHD at week 4 post graft compared to isotype control. Such markers include the T cell marker CD4, pro-inflammatory cytokines TNF-alpha, and Th2 cell differentiation regulator IL-33 (FIG. 46). This is consistent with reduction of infiltrating lymphocytes documented in histological analysis (FIG. 39). Of note, expression of CD4 and T cell adhesion molecules VCAM-1 highly correlated in skin (FIG. 48). This suggests that MEDI-579-induced reduction of VCAM-1 mediates, at least in part, the beneficial effect of MEDI-579 on the inflammation component of GVHD in skin.

### Fibrosis

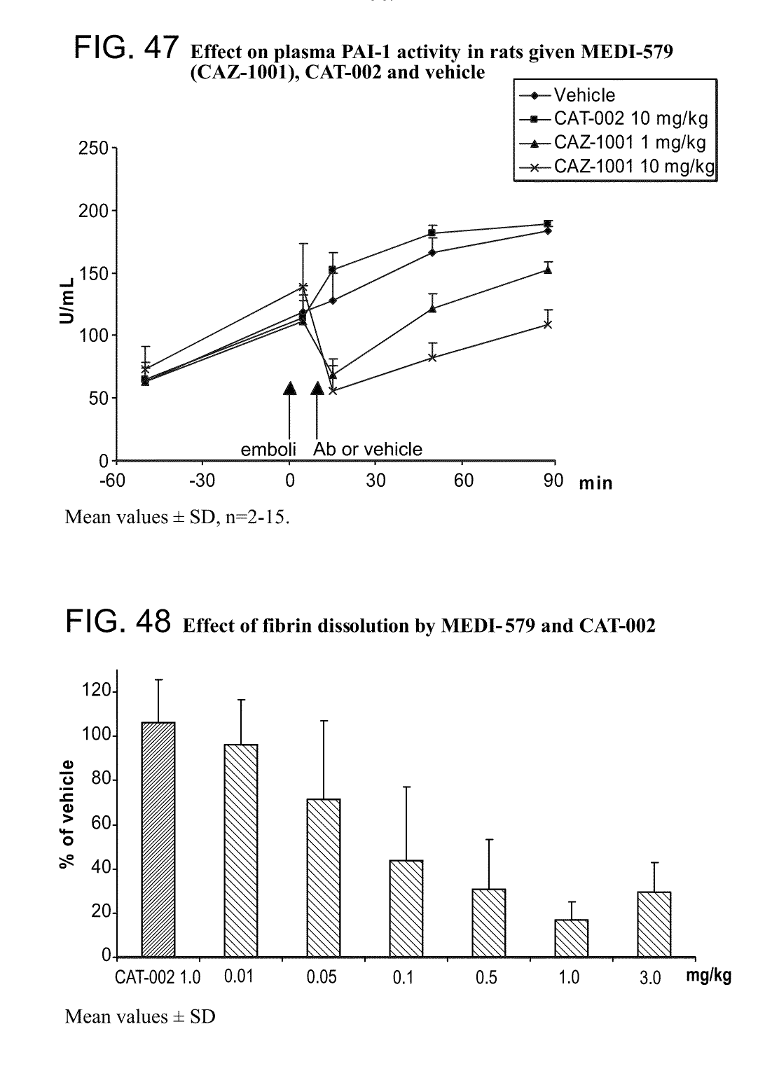

Prophylactic treatment with muMEDI-579 also significantly reduced gene expression of key fibrotic markers, MMP inhibitor TIMP-1 and pro-fibrotic Th-2 cytokine regulator IL-33, in murine GVHD compared to isotype control at week 4 post graft (FIG. 47). Of note, TIMP-1 and TNF-alpha expression highly correlated in skin (FIG. 48). This suggests that MEDI-579-induced reduction of TNF-alpha expression, and in general the inflammatory component of GVHD, at least in part mediates the beneficial effect of MEDI-579 on the fibrotic component of GVHD in skin.

Mechanism of Action of muMEDI-579 in Murine GVHD in Skin

FIG. 48 illustrates a general and hypothetical mechanism of action of MEDI-579 on three key pathological aspects of GVHD: inflammation, vasculopathy and fibrosis. This model emphasizes the pro-fibrinolytic function of muMEDI-579. Splenocyte transfer initiates the inflammation that in turn causes both vasculopathy and fibrosis through the release of mediators (FIG. 48A). The vasculopathy component of GVHD has a positive feedback onto inflammation and T cell infiltration via T cell adhesion molecules such as VCAM-1 (FIG. 48a). Blockade of PAI-1 by muMEDI579 promotes plasmin-based fibrinolysis, resolving vascular injury and activation. Resolution of vascular activation in turn reduces its impact on inflammation via VCAM-1 (FIG. 48A). Consistent with that view, CD4 and VCAM-1 mRNA expression correlates (FIG. 48C). Mice dosed with muMEDI-579 cluster at bottom range of scale, unlike mice dosed with isotype control that cluster at higher ranges with higher VCAM and CD4 levels. As pro-inflammatory TNF-alpha is reduced, fibrosis resolves. Consistent with that view, TIMP-1 and TNF-alpha highly correlates (FIG. 48B). Mice dosed with muMEDI-579 cluster at bottom range of scale, unlike mice dosed with isotype control that cluster at higher ranges with higher TIMP-1 and TNF-alpha levels.

### (II) THERAPEUTIC STUDY.

PAI-1 Blockade by muMEDI-579 Reduces Clinical Skin Score in Murine GVHD

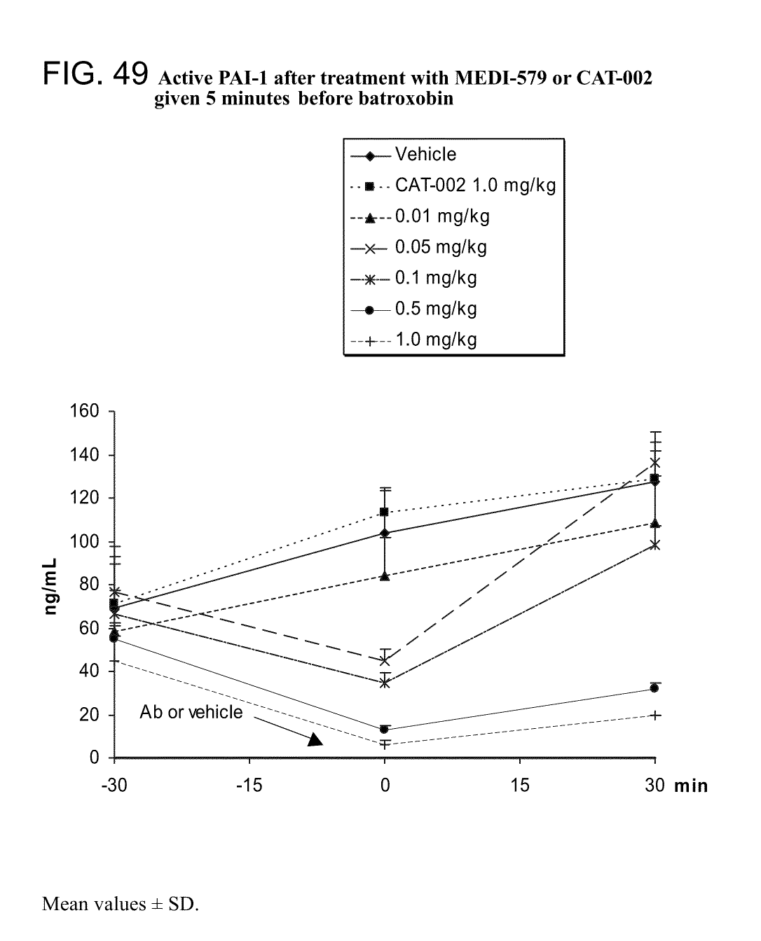

Therapeutic efficacy of muMEDI-579 was tested in a single dose study in murine GVHD with dosing starting at week 3 post graft, a point in time where GVHD is already well established (FIG. 49). muMEDI-579 at 10 mg/kg in this therapeutic setting significantly reduced clinical skin score, as early as one week after dosing starts, compared to isotype control (2.26-fold at termination, week 5½, p=0.01).

PAI-1 Blockade by muMEDI-579 Reduces Histology Skin Score in Murine GVHD

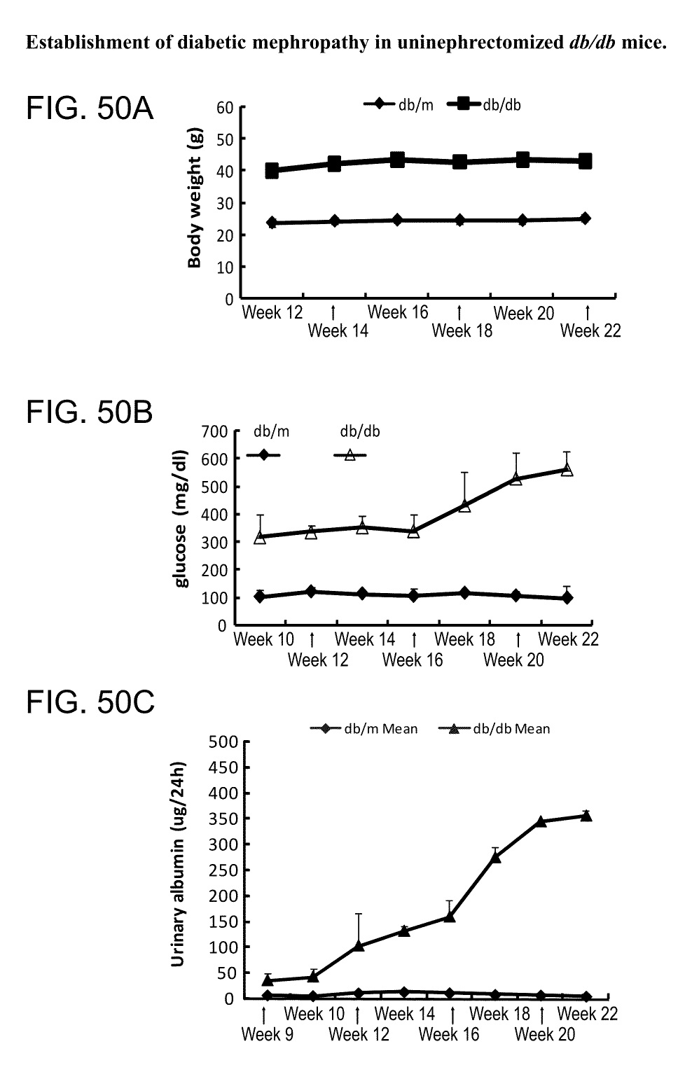

Therapeutic efficacy of muMEDI-579 on histology skin score was assessed in murine GVHD in back and ear at termination (5½ week post graft) in the same therapeutic setting. muMEDI-579 at 10 mg/kg significantly reduced histology skin score in both back and ear compared to isotype control (FIG. 50, p=0.0061 in back skin and p=0.0001 in ear skin). This reduction was based on reduced epidermal thickness and dermal fibrosis in back skin (FIG. 50B), and reduced epidermal thickness and ulcer incidence in ear skin. Heavier weight of the fibrotic component of the histology skin score in therapeutic compared to prophylactic studies is likely a direct consequence of reading point that comes later within the course of GVHD (5½ weeks for therapeutic vs. 4 weeks for prophylactic, post graft) when fibrosis is peaking (fibrosis peaks at week 6, following inflammation that peaks at week 4-Ruzek et al., Arthritis and Rheumatism 2004, 50:1319).

PAI-1 Blockade by muMEDI-579 Normalizes Fibrinolysis in Murine GVHD in Skin

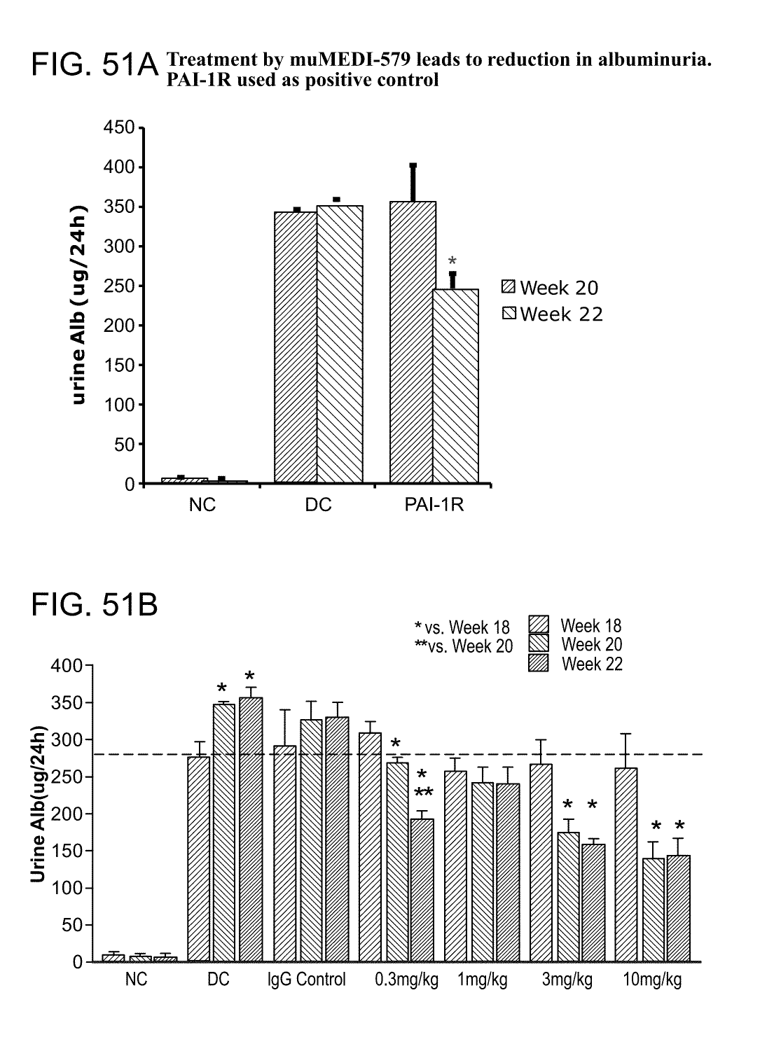

Therapeutic treatment with muMEDI-579 significantly reduced protein levels of active PAI-1, active plasmin and fibrin(ogen) locally in skin in murine GVHD compared to isotype control at week 5½ post graft. This reduction was also detected in systemic plasma compartment for plasmin (FIG. 51). Similar to prophylactic studies, reduction of these fibrinolysis pathway markers in murine GVHD by fibrinolytic agent muMEDI-579 in this therapeutic study is certainly reflective of a long-term, progressive beneficial impact of PAI-1 blockade on global GVH pathology in this chronic, non acute model: muMEDI-579 achieved normalization of fibrinolysis back to normal basal levels, as defined by control syngeneic graft.

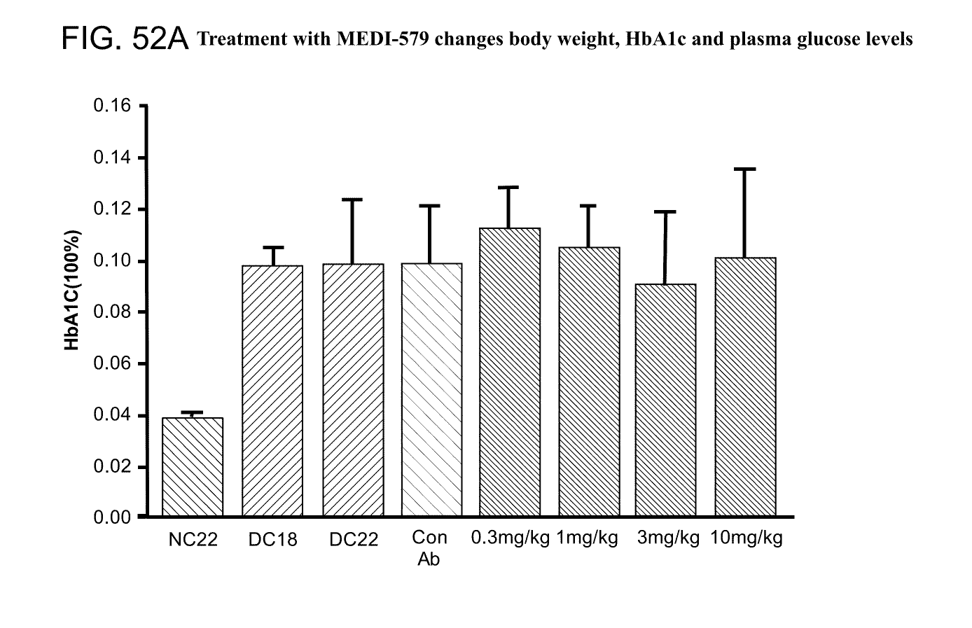

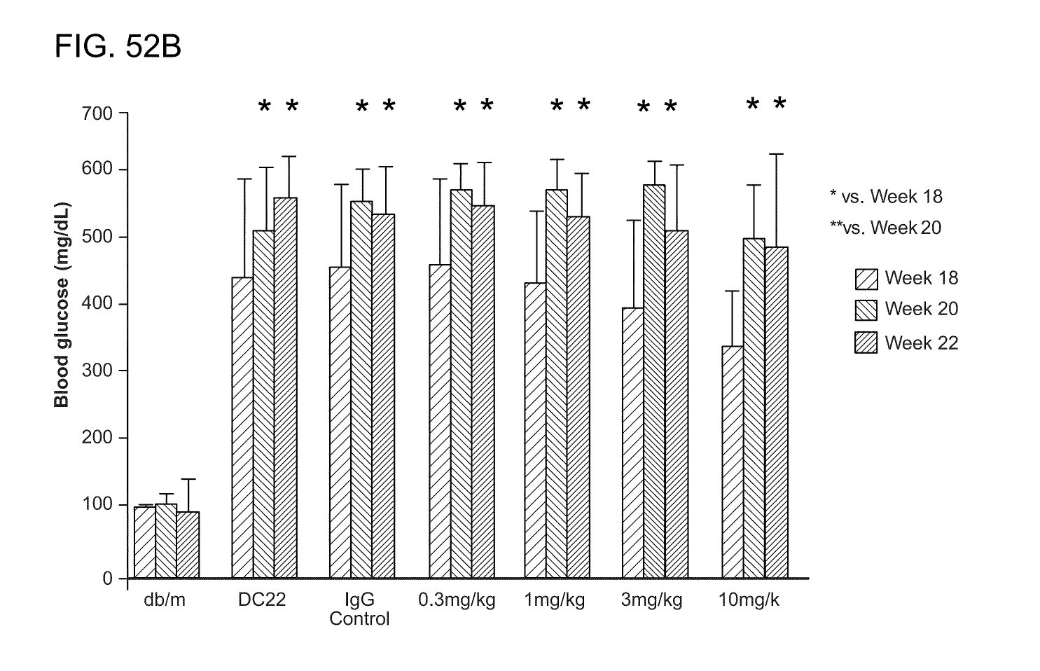

Gene profiling in skin revealed extended impact of muMEDI-579 on genes involved in the regulation of fibrinolysis, fibrosis, coagulation, inflammation and vascular injury (FIG. 52). KLK6 and KLK9 are among the genes whose expression can be modulated by muMEDI-579 treatment. KLKs activate signaling via the kallikrein-kinin system, PARs, and uPA and by processing of TGFβ and IGF-binding proteins. Aberrant regulation of KLKs has been associated with diverse diseases such as hypertension, renal dysfunction, skin disorders, and inflammation.

### MEDI-579 Stimulates Plasmin-Dependent MMP-1 Activation in Human Microvascular Endothelial Cells

In the following studies, a cell-based system was utilized to investigate the effect of MEDI-579 on stimulation of plasmin-dependent MMP-1 activation.

In one study, primary endothelial cells (HMVEC-dBlAd; Lonza, Cat. CC-2811) were plated overnight in 96 well plate at a concentration of 10,000 per well. Cells were treated with an anti-human PAI-1 antibody or isotype control for 15 minutes. Cells were then treated with recombinant human tumor-necrosis-factor α (TNF α; Invitrogen, Cat. PHC3011□□) at a concentration of 5 ng/ml and Plasminogen Lys-type (Calbiochem, Cat. 528185) at a concentration of 10 ug/ml. After 24 hours, the plate was centrifuged at 1150 RPM for 5 minutes. Supernatants were taken off and assayed for active MMP1 with a Fluorokine Assay (R&D Systems, F1M00) according to the manufacturer's instructions.

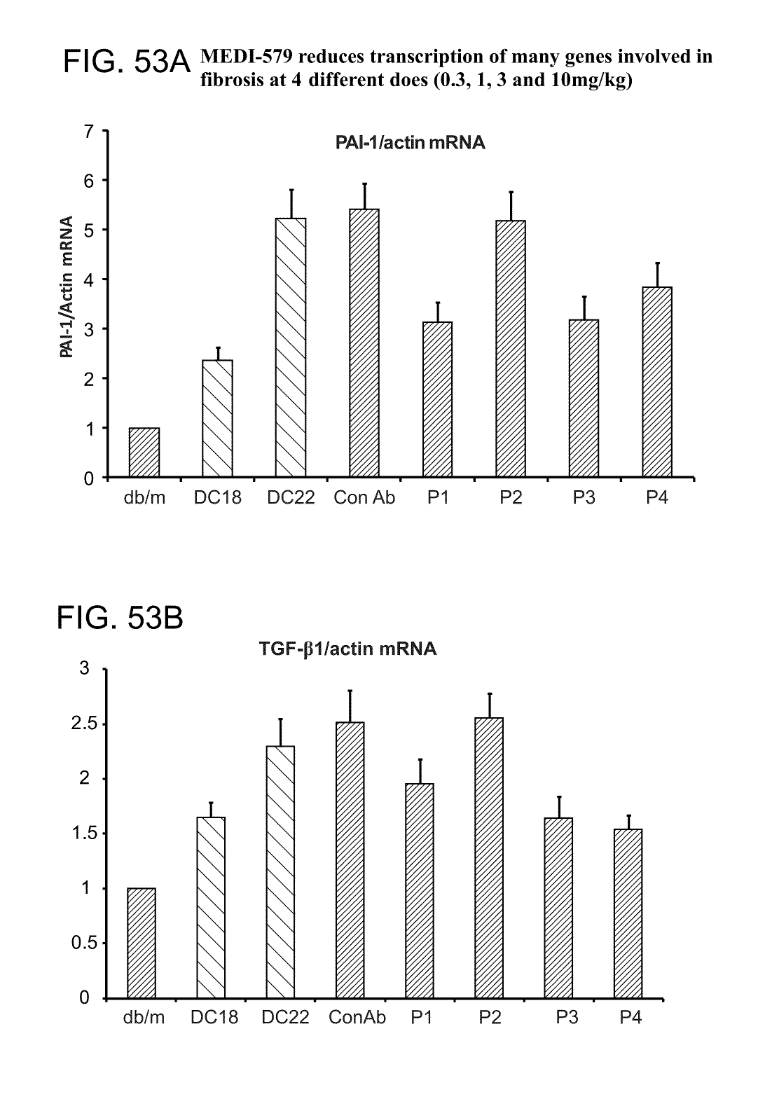

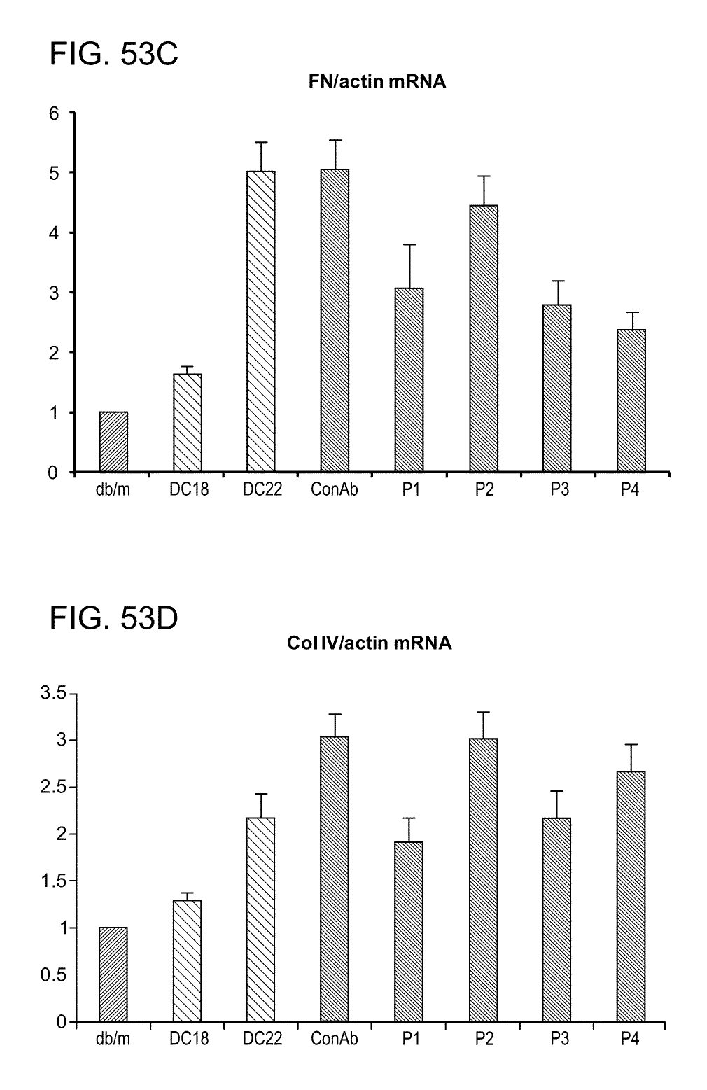

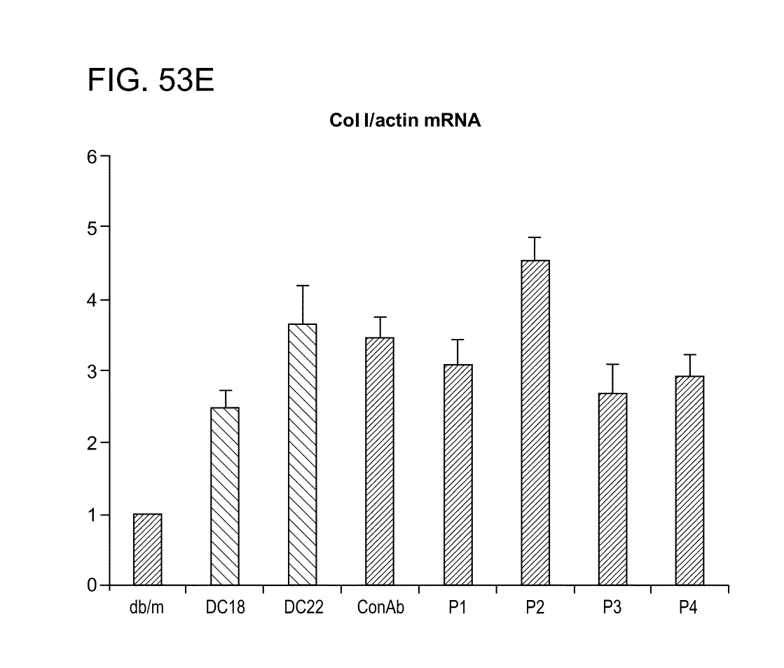

This study suggests that MEDI-579 stimulates plasmin-mediated MMP-1 activation in a concentration-dependent manner (FIG. 53). Failure of MEDI-579 to stimulate MMP-1 activation without plasminogen demonstrates that stimulation by MEDI-579 is a plasmin-mediated phenomenon. These results support the anti-fibrotic activity observed in the in vivo models.

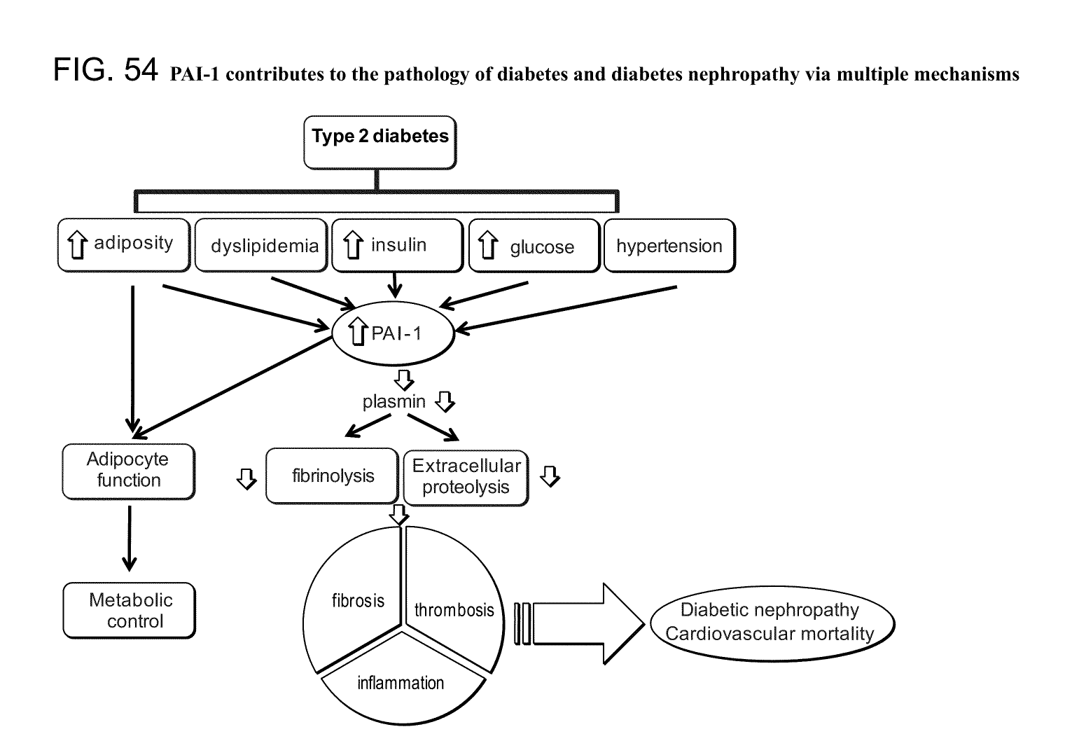

In a separate study, human lung fibrobasts (CCL-135 cells) and rat mesangial cells were treated with wt PAI-1, MEDI-579, or both MEDI-579 and wt PAI-1. When both MEDI-579 and wt PAI-1 were added to the cells for 72 h, the inhibition of ECM degradation seen with wt PAI-1 was reversed by MEDI-579 in a dose-dependent manner (FIG. 54). Thus, MEDI-579 promoted extracellular matrix (ECM) degration in lung fibroblasts and kidney mesangial cells by a plasmin-dependent process. Study design and methods for the present example are detailed below.

### Induction of GVHD

On study day 0, spleens from mouse strain B10.D2-Hc1H2dH2-T18c/nSnJ (B10.D2, Jackson Labs) mice were harvested and a cell suspension made by dispersion and passage through a 70 microns pore filter. Red blood cells were lysed by incubation for approximately 3-5 minutes in ice cold ammonium chloride solution (Stem Cell Technologies), and splenocytes pelleted by centrifugation for 5 minutes. Splenocytes (30×10⁶ cells) in 0.2 ml sterile phosphate buffered saline (PBS) pH 7.4 (Gibco) were then injected into the lateral tail vein of congenic BALB/c RAG2−/− mice (Taconic). Control syngeneic grafts were also carried out by grafting BALB/c splenocytes into BALB/c Rag 2−/− mice.

Dosing of Murine Anti-PAI-1 Antibody muMEDI-579

RAG2−/− mice were placed into treatment groups and administered 0.1 ml sterile IgG1 (either 200 micrograms anti-murine PAI-1 muMEDI579 or isotype control Colin−10 mg/kg) or PBS via intraperitoneal injection on day of graft (prophylactic dosing) or at week 3 post graft (therapeutic dosing). Mice were dosed twice weekly until termination of study at end of week 4 (prophylactic dosing) or at end of week 6 (therapeutic dosing)

### Clinical (In-Life) Scoring

Baseline clinical skin scores and proteinuria measurements were obtained on a biweekly basis from week 1 until termination, when animals were euthanized for tissue harvesting. Skin was clinically scored as indicated in table below:

| | | Score | |
|---|---|---|---|
| Swelling | eyes | 0.3 | pt |
| | paws | 0.3 | pt |
| Scarring | ears | 0.3 | pt |
| | tail | 0.3 | pt |
| skin lesion | <1 cm | 1 | pt |
| (hair loss) | >1 cm | 2 | pts |

Proteinuria was measured by Chemstrip (Roche), with a score of 1=30 milligrams/deciliter (mg/dL) mg/dl; 2=100 mg/d; 3=500 mg/d.

### Histopathology Scoring

Skin was harvested at study termination from identical dorsal locations of mice dosed with muMEDI-579 or isotype control. Histology skin score were established by a pathologist blinded to the treatment regimen from Hematoxylin and Masson's trichrome stained sections, based on the following scale:

| | | Histology | |
|---|---|---|---|
| | | Score | |
| T-cells | Sparse | 1 | pt |
| infiltration | Moderate | 2 | pts |
| | Marked | 3 | pts |
| | Heavy | 4 | pts |
| Epidermis | Focal | 1 | pt |
| thickening | Moderate | 2 | pts |
| | Marked | 3 | pts |

| | | |
|---|---|---|
| Fibrosis (Masson's | 3 | pts |
| trichrome) | | |
| Ulcers | 3 | pts |

### ELISAs for Plasma and Skin Biomarkers

Levels of fibrinogen, plasmin and PAI-1 were determined by ELISA according to the manufacturer's protocol (Innovative Research). For the detection of fibrinogen, plasma was diluted 1:10,000 while skin lysates were run diluted 1:100 in PBS. For plasmin, plasma was diluted 1:1000 and skin lysates were diluted 1:50 in PBS. For PAI-1, plasma and skin lysates were assayed neat. For capture of the respective target protein, the fibrinogen ELISA used an anti-fibrinogen antibody, the plasmin ELISA used alpha 2-antiplasmin, and the active PAI-1 ELISA used uPA coated onto a 96-well plate.

Total RNA Extraction and Fluidigm analysis

Total RNA was purified from skin biopsies using the Qiagen RNeasy Tissue Mini kit (Hilden, Germany) after rotator-homogenization of the tissues. RNA purity and concentration were determined spectrophotometrically (260/280 >1.9). 500 ng of RNA were retrotranscribed into cDNA and then preamplified using the TaqMan PreAmp Master Mix Kit (Applied Biosystems). Samples were run in duplicate using TaqMan Gene Expression Assays in the BioMark 48.48 Dynamic Array chips (Fluidigm Corp). Dynamic arrays were loaded using a NanoFlex 4-IFC Controller (Fluidigm Corp), and real-time reactions were performed and analyzed using the BioMark Real-Time PCR System and Analysis software (Fluidigm Corp), respectively. SDs were calculated for each set of duplicate reactions and then averaged for each dynamic array. CTs above 30 were excluded from the calculation. Delta-delta Cts (ΔΔCt) were calculated using the mean of the 3 reference genes (GAPDH, PPIA, Beta-2) and a calibrator sample, and were converted to fold expression change by the following formula: 2−ΔΔCt.

### Test Animals

| | |
|---|---|
| Species | Mouse |
| Strain | 129S6(B6)-Rag2^{tm1Fwa}N12 |
| Substrain | N/A^{a} |
| Age range | 8-12 weeks |
| Body Weight range | 19-24 gramsg |
| Supplier | Taconic |

^{a}Not Applicable

### Example 16

### Efficacy of MEDI-579 on Arterial Thrombosis in Rats

The present study shows that MEDI-579 affected thrombus size. The dose 10 mg/kg resulted in approximately 30% smaller thrombi when harvested 180 minutes (p=0.0154) after thrombus induction. Time to occlusion and arterial blood flow was not affected. Active PAI-1 in plasma was reduced. The isotype control antibody CAT-002 had no effect on thrombus size, time to occlusion, arterial blood flow or active PAI-1 in plasma.

### Detailed findings of the present study are described below

Effect on Thrombus Size (RTS), Time to Occlusion (TTO) and Active PAI-1 after 10 Mg/Kg of MEDI-579 and Thrombus Harvest after 180 Minutes

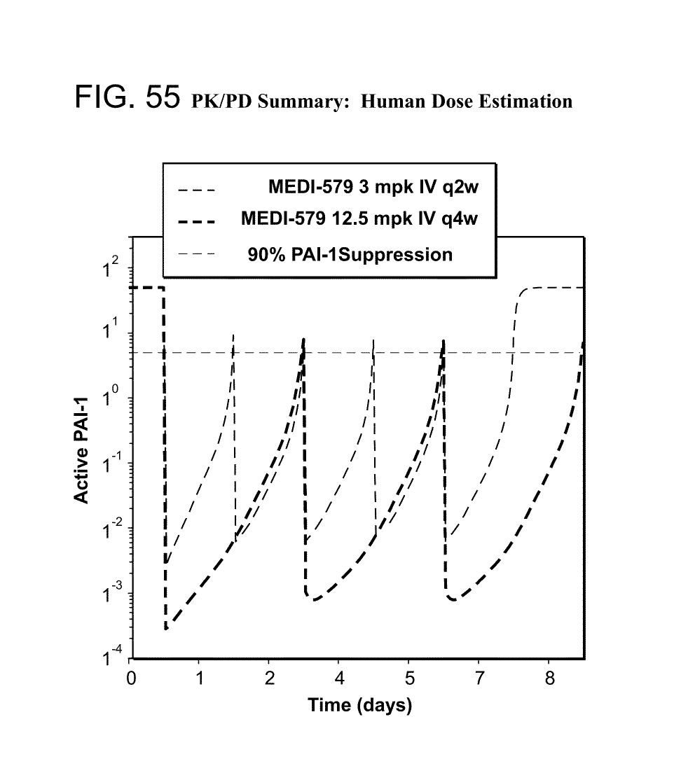

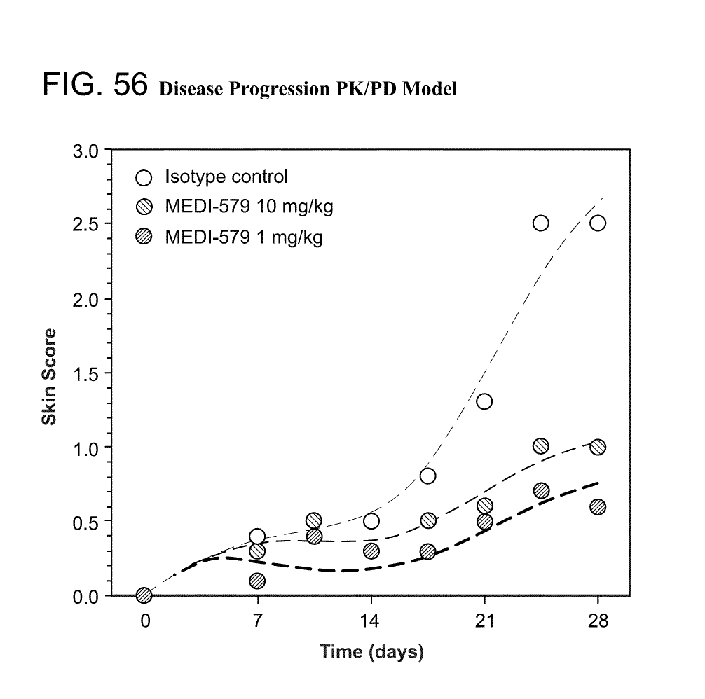

The effect on thrombus size is shown in FIG. 55. There was a significant reduction (31%) in thrombus size after administration of MEDI-579 compared to vehicle rats (p=0.0154). In addition, in ex vivo clot lysis assay, MEDI-579 also stimulated clot lysis in human plasma ex vivo, while CAT-002 had no effect (FIG. 56). The EC50 for stimulation of clot lysis was about 1 nM.

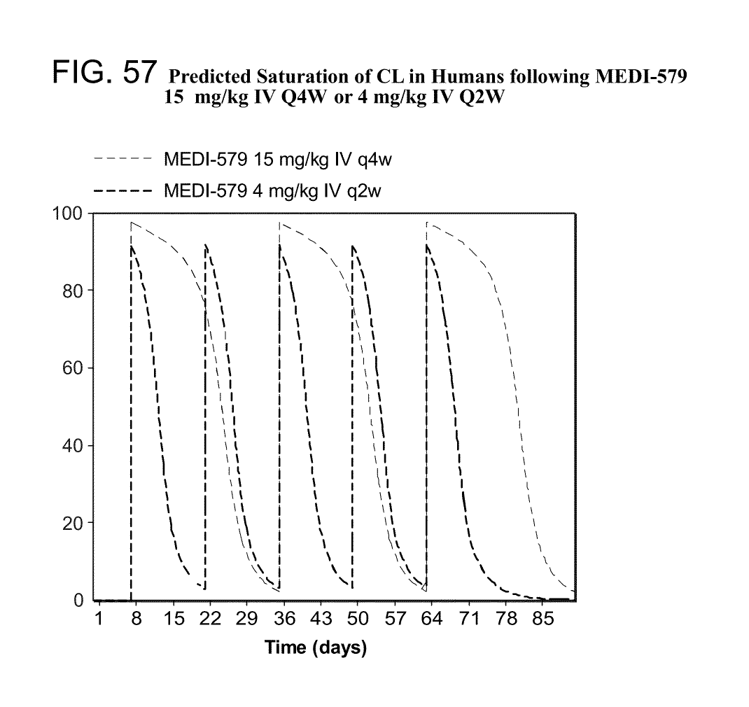

The effect on TTO is shown in FIG. 57. TTO showed a prolongation of 2.7 minutes in rats given MEDI-579 compared to the vehicle rats. The prolongation was not significant (p=0.1276).

After administration of MEDI-579, there was a reduction in active PAI-1 in plasma to approximately 0.10 ng/mL 30 minutes after FeCl₃ application (FIG. 58). During the following 150 minutes, active PAI-1 increased to 0.28 ng/mL. Vehicle rats showed an increase in active PAI-1 over time. The difference in active PAI-1 between rats given MEDI-579 and vehicle rats was the same both 30 and 180 minutes after FeCl₃ application.

Study design and methods for the present example are detailed below.

### Animal Preparation

Anaesthesia was induced by inhalation of isoflurane for about 1 minute. When anaesthetized, the rats were given thiobutabarbital sodium salt, 100 to 120 mg/kg intraperitoneally (ip), in order to maintain the anaesthesia. One catheter was inserted in the left jugular vein for administration of ¹²⁵I-fibrinogen, antibody or vehicle. The right carotid artery was gently detached from surrounding tissue and a thread was placed around it. For better access to the vessel, the muscle in front of the artery was ligated and removed, whereupon lukewarm saline was sprayed over the area. The rats were tracheotomized in order to facilitate spontaneous breathing. One catheter was also inserted in the left femoral artery for blood sampling and mean arterial blood pressure and heart rate recordings. To avoid blood clots in the arterial catheter, a slow saline infusion was maintained throughout the experiment. The body temperature was monitored and maintained at 38° C. by external heating. A Doppler flow probe was hooked around the carotid artery for blood flow measurements. After the preparation, the rats were left to recover for 20 to 30 minutes. Pressure signals were registered on a PC, where a custom-made computer program sampled data from blood pressure and heart rate at defined time intervals.

### 1. Arterial Thrombosis

Twenty minutes before FeCl₃ application, ¹²⁵I-fibrinogen, ˜55 kBq/animal, was given as an iv injection. Blood samples for determination of ¹²⁵I were taken 5 minutes before and 55 minutes after FeCl₃ application. Blood was drawn into plastic tubes. MEDI-579 or saline was administered as an iv injection 10 minutes before FeCl₃. Dalteparin was given as a sc injection in the neck 20 minutes after FeCl₃ application to inhibit clot accretion.

The external surface of the carotid artery was carefully dried with cotton tipped applicators. A filter paper (diameter 3 mm), was placed on an in-house made plastic holder and soaked with 1.5 μL to 1.7 μL FeCl₃. At experimental time 0, the flow probe was removed and the artery was placed in the cavity of the holder and small triangular swabs were placed under the holder, ensuring that the vessel area in contact with FeCl₃ was restricted to the filter paper area and that a part of the vessel circumference was left unaffected. After 5 minutes, the filter paper together with the holder was removed and the artery was sprayed with lukewarm saline.

The experiments were finalized 180 minutes after thrombus induction. The right carotid artery was excised and the vessel with the thrombus was gently washed in saline and blotted after which the artery+thrombus was placed in an empty test tube. Blood samples and the artery with thrombus were analysed with respect to the ¹²⁵I-content, using a gamma counter and the relative thrombus size (RTS artery+thrombus) was calculated.

### 2. Blood Sampling

Blood samples for determination of active PAI-1 were taken at experimental time—25, 30, 90 and 175 minutes. Blood was drawn into plastic tubes containing 0.109 M trisodium citrate. The blood samples were centrifuged at 10000 g for 5 minutes at 22° C. Plasma was separated and stored at −20° C. until analysed (RPAIKT, Molecular Innovations Inc, USA).

The experimental protocol is summarized in the chart below.

### Example 17

### Efficacy of MEDI-579 on Pulmonary Embolism (PE) in Rats

The present study shows that MEDI-579, at 1 and 10 mg/kg iv, stimulated endogenous fibrinolysis in this model. The effect on lung emboli reduction was significant 90 minutes after the injection of the experimental emboli. The isotype antibody CAT-002, at 10 mg/kg iv, had no corresponding effect on the experimental emboli.

As measured using the Specrolyse kit, plasma PAI-1 activity decreased after administration of MEDI-579 in a dose-dependent manner. Both doses decreased the activity to the same extent but the PAI-1 activity reduction in the rats given the highest dose remained at the same level during the experiment while in the rats given the lower dose, the activity reduction declined over time.

Detailed findings of the present study are described below.

### Endogenous Fibrinolysis in Vehicle Rats

In vehicle rats the endogenous fibrinolysis dissolved the lung emboli gradually. Of the administered 125I-activity, 78% was trapped in the lungs 5 minutes after the clot injection and hence, this level was set to 100% (control) when calculating the effect on the fibrinolysis. During the following 90 minutes, 125I-activity decreased to 76% compared with the control value (FIG. 59) i.e., 24% of the clotted fibrin was dissolved.

### Effect of MEDI-579 on Emboli Reduction

The effect of MEDI-579 was determined 90 minutes after injection of the experimental emboli. MEDI-579 reduced the lung emboli with 42% (p=0.05) and 46% (p=0.01) compared with the vehicle group after administration of 1.0 mg/kg and 10.0 mg/kg, respectively (FIG. 60). In rats given CAT-002, the emboli size was not reduced.

### Effect of MEDI-579 on Plasma PAI-1 Activity

LPS administration resulted in increased plasma PAI-1 activity. Basal level of PAI-1 activity in the rat is around 10 U/mL (data not shown) and 240 minutes after LPS, i.e., at the end of the experiment, the plasma level in vehicle- and CAT-002 treated animals was around 180 U/mL.

Plasma PAI-1 activity was dose-dependently decreased after administration of MEDI-579. Both doses decreased the activity from around 120 U/mL to 55 U/mL, thus with 65 U/mL. The PAI-1 activity reduction in the rats given the highest dose remained at the same level, i.e., 65 U/mL during the experiment while in the rats given the lower dose, the activity reduction declined over time (FIG. 61).

Study design and methods for the present example are detailed below.

### Animal Preparation

Anaesthesia was induced and maintained by administration of thiobutabarbital sodium salt 100-120 mg/kg given intraperitoneally (ip) to the rats. Two catheters were inserted in the left jugular vein, one for injection of the blood clot and the other catheter for administration of MEDI-579, the control antibody CAT-002 or vehicle. A catheter was introduced into the carotid artery for blood sampling and mean arterial blood pressure and heart rate recordings. To avoid blood from clotting in the arterial catheter, a slow saline infusion was maintained throughout the experiment. The rats were tracheotomized in order to facilitate spontaneous breathing. The body temperature was monitored and maintained at 38° C. by external heating. After the preparation, the rats were left to recover for about 30 minutes. Pressure signals were registered on a PC where the program PharmLab sampled data from blood pressure and heart rate at defined time intervals.

### Sample Preparation

The rats were given an iv injection of LPS at the start of the experiment, i.e., 150 minutes before clot injection. Ninety minutes later, i.e., 60 minutes before clot injection, approximately 0.5 mL blood from each rat was drawn into a plastic test tube. From this test tube, 0.4 mL blood was transferred to a second test tube, 125I-fibrinogen was added and the tube content was gently mixed. Of this 125I-fibrinogen and blood mixture, 0.3 mL was drawn into a syringe, which was left in room temperature for 60 minutes for the blood to clot. At experimental time 0, the blood clot was injected into vena jugularis followed by 0.2 mL saline flushing. The injection of MEDI-579, CAT-002 or vehicle was given 10 minutes after clot injection. The experiments were completed 90 minutes after the clot injection and the lungs were excised, the tissue gently washed in saline and thereafter blotted and weighed. The 125I-activity in the syringe was determined both before and after the clot injection in order to obtain a value of total given 125I-activity. The lung tissue were also analysed with respect to the 125I-content, using a gamma counter.

Blood samples for determination of PAI-1 activity were taken 50 minutes before and 5, 15, 50, and 90 minutes after clot injection. Blood was drawn into plastic tubes containing 0.129 M trisodium citrate. The blood samples were centrifuged at 10000 g for 5 minutes at 22° C. Plasma was separated and stored at −20° C. until analysed (Spectrolyse (PL) PAI, American Diagnostica, Stamford, Conn., USA).

The experimental protocol is summarized in the chart below.

### Example 18

### Efficacy of MEDI-579 on Fibrin Dissolution in DIC Model Rats

The present study demonstrates that MEDI-579, administered as iv-bolus, improves the dissolution of formed fibrin clots in the anaesthetized rat in this DIC model. The ED₅₀ dose is in the range of 0.05 and 0.1 mg/kg corresponding to a plasma concentration of 4-10 nmol/L. The isotype antibody CAT-002 has no effect on fibrin dissolution in this model.

Five minutes after administration of MEDI-579, active PAI-1 is decreased in a dose dependent manner. However, this decrease declines in the following 30 minutes. CAT-002 has no effect on the level of active PAI-1.

Detailed findings of the present study are described below.

Fibrin deposition in the lung and fibrinogen or fibrin degradation products in plasma of control rats given only batroxobin or LPS and batroxobin has earlier been evaluated showing that LPS leads to deterioration of the endogenous fibrinolysis.

Measuring the amount of ¹²⁵I-activity in the lungs 5 minutes after batroxobin administration demonstrated that the rats given MEDI-579 had similar fibrin accumulation in the lungs as the vehicle rats, indicating that MEDI-579 does not influence batroxobin-induced clot formation.

### Effect on Fibrin Dissolution

The amount of 125I in the lungs, reflecting the deposited fibrin, was measured 30 minutes after batroxobin administration. The antibodies were administered 5 minutes before batroxobin. In the rats treated with MEDI-579, 125I-activity in the lungs decreased in a dose dependent manner (FIG. 62) indicating improved dissolution of deposited fibrin. The ED50 dose was in the range of 0.05 and 0.1 mg/kg, corresponding to a plasma level of 4 to 10 nmol/L. The control antibody CAT-002, 1 mg/kg iv, did not affect the fibrin deposition.

### Effect on Active PAI-1 in Plasma

The level of active PAI-1 decreased in a dose dependent manner 5 minutes after administration of MEDI-579. During the following 30 minutes, active PAI-1 was increased with no inhibition for the lower doses, but was kept on a low level (75 and 85% inhibition, respectively) after the two highest doses (FIG. 63). No reduction in active plasma PAI-1 was shown after administration of CAT-002.

Study design and methods for the present example are detailed below.

### Animal Preparation

Anaesthesia was induced and maintained by administration of thiobutabarbital sodium salt 100-120 mg/kg given intraperitoneally (ip) to the rats. Two catheters were inserted in the left jugular vein for administration of LPS, 125I-fibrinogen, batroxobin and MEDI-579, the control antibody CAT-002 or vehicle. Another catheter was inserted in the right carotid artery, for blood sampling and mean arterial blood pressure and heart rate recordings. To avoid blood clots in the arterial catheter a slow saline infusion was maintained throughout the experiment. The rats were tracheotomized in order to facilitate spontaneous breathing. The body temperature was monitored and maintained at 38° C. by external heating. After the preparation, the rats were left to recover for 20-30 minutes. Pressure signals were registered on a PC, where a custom made computer program sampled data from blood pressure and heart rate at defined time intervals.

### Sample Preparation

Disseminated intravascular coagulation (DIC) was induced in rats. The animals were given an iv injection of LPS at the start of the experiment, i.e., 150 minutes before administration of the pro-coagulant enzyme batroxobin. ¹²⁵I-fibrinogen was given as an iv injection 15 minutes before administration of batroxobin. MEDI-579, CAT-002 or vehicle was given as an iv injection 5 minutes before administration of batroxobin. Batroxobin was given as an iv injection at experimental time zero.

Blood samples for determination of ¹²⁵I-activity were taken in all experiments just before and 5, 20 and 30 minutes after the batroxobin administration. At the end of the experiment, the lungs were excised, the tissue was gently washed in saline and thereafter blotted and weighed. The ¹²⁵I-activity in the tissue and blood samples was determined, using a gamma counter.

Blood samples for determination of active PAI-1 in plasma were taken at experimental time zero as well as 30 minutes before and 30 minutes after experimental time zero, i.e., at the end of the experiment. Blood was drawn into plastic tubes containing 0.129 M trisodium citrate. The blood samples were centrifuged at 10000 g for 5 minutes at 22° C. Plasma was separated and stored at −20° C. until analysed.

The experimental protocol is summarized in the chart below.

### Example 19

### Efficacy of MEDI-579 on Diabetic Nephropathy in Uninephrectomized Db/Db Mouse Model

Without being bound by theory, PAI-1 may contribute to the pathology of diabetes and diabetic nephropathy via multiple mechanisms (FIG. 68). PAI-1 influences the balance of ECM turnover towards ECM deposition by inhibiting plasminogen activators and thus reduces plasmin activity. As shown herein, reducing PAI-1 activity by neutralizing antibody or PAI-1 dominant mutant (PAI-1R) decreased glomerular matrix accumulation and prevented the progression of renal fibrosis in rodent diabetic nephropathy models as well as other renal fibrosis models. This was also followed by concomitant improvement of albuminuria/proteinuria and reduction of ECM components in kidneys. In addition to its classic role in proteolysis, PAI-1 inhibition may also reduce the synthesis of ECM component as well as PAI-1 itself in rodent renal injury models. This suggests that PAI-1 also exerts a plasmin-independent mechanism in reducing fibrosis. Accordingly, inhibiting PAI-1 by administration of anti-PAI-1 antibodies, such as MEDI-579, to achieve higher plasmin level and ECM turnover as well as reducing synthesis of ECM components could confer significant benefits in treatment of diabetic nephropathy.

The db/db mice (leptin receptor deficiency) on the C57BLK6 background have been investigated intensively as a diabetic nephropathy model and exhibit many features similar to human diabetic nephropathy. Of the many mouse models of diabetes identified, the db/db mouse appears to most closely mimic the progressive nature of mesangial matrix expansion seen in human diabetic nephropathy. Recent studies suggest that uninephrectomy hastens the development of albuminuria in db/db mice, which provides an effective window for evaluating therapeutic intervention.

The present study evaluated the therapeutic principal of PAI-1 inhibition by MEDI-579 on diabetic nephropathy (DN) parameters using the uninephrectomized db/db mice as a model. This study was designed to address the effects of MEDI-579 treatment alone as well as the effects in combination with an ACE inhibitor, enalapril. The effects include the potential of MEDI-579 in preventing progression of DN or reversal of established DN.

Administration of a positive control, PAI-1R protein is as described in Huang et al. JASN 19:329-338, 2008, which is incorporated by reference in their entirety.

Detailed findings of the present study are described below.

### Establishment of Diabetic Nephropathy in Uninephrectomized Db/Db Mice

The diabetic gene (db) is transmitted as an autosomal recessive trait and encode for a G-to-T point mutation of the leptin receptor, leading to abnormal signaling of leptin. Diabetic nephropathy develops gradually with progressive increase of hyperglycemia which can be noted as early as 10 days-old mice. The intact db/db mice develop a mild form of DN over about 6 months of time. Uninephrectomy hastened the development of DN before the diabetes became too severe and allowed a window of time to study DN. Body weight (FIG. 64A), plasma glucose (FIG. 64B), urinary albumin excretion (FIG. 64C) and histology (pending) were monitored for 22 week period of time for both db/m and db/db mice and the results are shown in FIG. 64.

Urinary albuminuria was increased after the onset of diabetes and progressed steadily. The most robust increase coincided with rapid increase of plasma glucose between 16 to 20 weeks. The robust changes of albumin excretion during this time frame allowed a window for testing therapeutic effects of therapeutic intervention.

### MEDI-579 Significantly Reduces Albuminuria in Uninephrectomized Db/Db Mice

The treatment of uninephrectomized db/db mice was initiated at week 18 and mice were administered with either an isotype control antibody (MNGC) or muMEDI-579 via i.p. Four different doses of muMEDI-579 (0.3, 1, 3 and 10 mg/kg) and the isotype control (10 mg/kg) were injected twice weekly for total treatment period of 4 weeks. Albuminuria was assessed by quantitative analysis of 24 hour urine samples collected by metabolic cages every two weeks. Treatment by muMEDI-579 (three doses) led to a significant reduction of albuminuria, which regressed to a level that is lower than that of week 18 (FIG. 65B). This effect suggests a restoration of the glomerular barrier properties. The effect of MEDI-579 on albuminuria was relatively rapid and was observed as early as two weeks into treatment. PAI-1R was used as a positive control since its renal protective effects was shown previously (FIG. 65A). Either PAI-1R (data not shown) or MEDI-579 treatment changed body weight, HbA1c and the plasma glucose levels significantly (FIGS. 66A and 66B).

### MEDI-579 Reduces Transcription of Many Genes Involved in Fibrosis

Total RNA was prepared from kidney cortex and mRNA encoding for may genes involved in fibrosis were measured and normalized to actin mRNA. These genes include PAI-1, TGF-β1, fibronectin, collagen type 1 and type IV. As shown in FIG. 67, all of these genes were up-regulated during progression of DN, particularly PAI-1 and fibronectin mRNA. MEDI-579 treatment clearly reduced mRNA levels for PAI-1, TGF-β1, and fibronectin.

### Additive Effects Observed With Co-Administration of Enalapril

Treatment of mice with both enalapril and MEDI-579 produced additive benefits. The combined treatment regimen resulted in a further reduction of albuminuria. In this experiment, mice uninephrectomized at week eight were treated with enalapril (10 mg/kg/day) beginning week 12 and with MEDI-579 (3 mg/kg) beginning week 18. Once the particular treatment began, it continued until study termination at week 22. This experiment indicated additional reduction albuminuria following combined treatment with both agents.

Study design and methods for the present example are detailed below.

Fifteen groups of mice were used in these experiments. The experiments were carried out in 2 phases with a 9-week time gap to evaluate preferable dosages of the active agents.

The various groups include the following:

1. 10 db/dm mice (Non-diabetic control)
2. 10 db/db mice sacrificed at week 18 (Disease control-1)
3. 10 db/db mice sacrificed at week 20 (Disease control-2)
4. 10 db/db mice sacrificed at week 22 (Disease control-3)
5. 10 db/db mice treated with Dose Isotype control Ab
6. 10 db/db mice treated with Dose 1 PAI-1 Ab (0.3 mg/kg)
7. 10 db/db mice treated with Dose 2 PAI-1 Ab (1 mg/kg)
8. 10 db/db mice treated with Dose 3 PAI-1 Ab (3 mg/kg)
9. 10 db/db mice treated with Dose 4 PAI-1 Ab (10 mg/kg)
10 db/db mice treated with PAI-1R as in Huang et. al.

The last 4 groups were started 9 weeks after an optimal dose of MEDI-579 was determined based on the primary determinations.

11. 10 db/m mice sacrificed at week 22 (Non-diabetic control)
12. 10 db/db mice sacrificed at week 18 (disease control-1)
13. 10 db/db mice scarified at week 20 (disease control-2)
14. 10 db/db mice treated with enalapril (week 12 to 22) are also treated by isotype IgG control from week 18 to 22
15. 10 db/db mice treated with enalapril (from week 12 to 22) are also treated by MEDI-579 (3 mg/kg) from week 18 to 22

### Animals

Diabetic db/db mice and their lean non-diabetic db/m littermates were purchased from the Jackson Laboratory (Bar Harbor, Me.) and housed in accordance with the National Institutes of Health Guide for the Care and Use of Laboratory Animals. The animal experiments were approved by the Animal Care Committee of University of Utah. The db/db mice are determined to be diabetic by the vendor on the basis of appearance of obesity at approximately 5 wk of age and are expected to be hyperglycemic when tested after arrival in our laboratory at week 7. All mice were subjected to right nephrectomy under anesthesia at week 8 to hasten the development of diabetic nephropathy.

At week 18, Group 2 mice were sacrificed to determine disease severity at week 18. Similar analyses were performed from the groups of mice scarified at week 20 as well as week 22.

### Treatment

Treatment began at week 18 for group 5, 6, 7, 8 and 9 and continued for 4 weeks. MEDI-579 was administered IP twice a week on days 1 and 4 of the week. Normal control mice received PBS injections.

Enalapril/MEDI-579 combination groups (group 11 to 15) were started 9 weeks later than the others to ensure adequate time to determine the optimal MEDI-579 dose we want to use. Enalapril treated mice received treatment when albuminuria reached more than 8 fold of db/m and were given via drinking water. (A small amount of Splenda≈1 teaspoon/100 ml bottle) were given to all mice. Water volume ingested in enalapril-treated and control mice were measured to check that their intake was not affected by the Splenda. Blood Glucose determinations were carried out every 2 weeks at 8, 10, 12, 14, 16, 18, 20 and 22 weeks (7 times). Urinary albumin levels were determined every 2 weeks from 8 weeks until 22 weeks. Each mouse was placed in a metabolic cage for 24 hours just prior to sacrifice. Urine collected was used to measure urinary albumin and creatinine.

### Sacrifice

Mice were sacrificed under isoflurane anesthesia. Blood samples were taken by heart puncture. Plasma samples were prepared for plasma HbA_{1C}, creatinine, active PAI-1 as well as MEDI-579 levels.

Kidneys were perfused through the heart with 30 ml of cold PBS and then excised. Renal cortex was harvested by dissection and either snap-frozen in 2-methylbutane at 80° C. or fixed in 10% neutralized formalin for immunohistologic examination. Pieces of cortex were treated 3 ways: stored in liquid nitrogen for Western blot and plasmin activity measurement, treated with TRIzol Reagent (GibcoBRL, Gaithersburg, Md.) for simultaneous isolation of RNA or treated with 100 mMNaC1 and 20 mM HEPES to be sonicated for 30s three times on ice and centrifuged at a high speed for 15 min at 4° C. The supernatant was then collected and stored at 80° C. for FN and TGFβ-1 ELISA and protein measurement by a BCA protein assay kit (Pierce, Rockford, Ill.)

### Endpoints

Primary readouts include (1) PAS staining and scoring; (2) Urinary Creatinine every 2 weeks; and (3) Microalbuminuria every 2 weeks. Secondary Readouts include (1) Fn ELISA; (2) TGF-β ELISA; and (3) rtPCR for TGF=β1, PAI-1, FN, Collagen I, collagen IV & β-actin. Immunohistochemical Staining includes (1) PAI-1 Ab staining; (2) Col I Staining; (3) Col IV Staining; and (4) FN staining. Other assays include (1) Hb A1C every 2 weeks; and (2) Plasmin activity.

### Example 20

### PK/PD Study Summary, Human Dose Estimation, and Formulation

### PK/PD Study Summary

MEDI-579 exhibited non linear PK in mice, rats and monkeys. The antigen sink is PAI-1 specific. Normal hAb clearance was demonstrated in a single dose PK study in PAI-1 KO mice.

A PK/PD model correlating exposure with target neutralization (active PAI-1 in the circulation) in monkeys was established. IC50 and IC90 for saturation of CL and target neutralization were estimated.

Translational simulation using the PK/PD model fitted to the monkey data was conducted (FIG. 69). The IV dose regimen in humans predicted to maintain approximately 90% neutralization of active PAI-1 is 3 mg/kg IV every 2 weeks or 12.5 mg/kg every 4 weeks. However, saturation of clearance is not maintained over the dosing interval.

### Human Dose Estimation

Data from the mouse SLE model indicated that 90% inhibition of active PAI-1 in the circulation achieved 70% inhibition of proteinuria (n=1 experiment).

A PK/PD model of disease progression for mouse GVHD pharmacology model was also established to correlate exposure with efficacy (FIG. 70). IC50 for effect on skin score was estimated at 0.5 μg/ml.

FIG. 71 shows predicted saturation of CL in humans following MEDI-579 administered at a dose of 15 mg/kg IV Q4W or 4 mg/kg IV Q2W.

If saturation of clearance is shown to parallel the concentration-response relationship for efficacy in the GVHD mouse model, higher doses than predicted might be required for efficacy as saturation of clearance is not maintained over the dosing interval.

The Table below shows correlation between improvement in kidney function and active PAI-1 inhibition in the circulation in the mouse model. The data from the SLE model suggested that 90% inhibition of active PAI-1 in the circulation might be required to achieve efficacy.

BN_08_62: muMEDI-579 in Adv-IFN-a Accelerated Lupus

| | | | Wk 5.0 | | Plasma | |
|---|---|---|---|---|---|---|
| | | | TP: Creat | | act. PAI-1 | |
| | | | (mg/dl/ | % | | % |
| Animal # | Treatment | | 24 hr) | inhibition | (ng/ml) | inhibition |
| Naïve | | mean | 6.79 | | 0.07 | |
| | | SD | 1.20 | | 0.05 | |
| Ad Null | | mean | 5.52 | | 0.03 | |
| | | SD | 0.81 | | 0.03 | |
| Adv-IFN | Isotype | mean | 68.64 | | 1.47 | |
| | 10mpk | SD | 35.67 | | 1.67 | |
| Adv-IFN | MEDI-579 | mean | 21.60 | 68.53 | 0.14 | 90.57 |
| | 10mpk | SD | 31.38 | | 0.14 | |
| Adv-IFN | MEDI-579 | mean | 40.63 | 40.80 | 0.48 | 67.69 |
| | 1mpk | SD | 43.95 | | 0.44 | |

### Formulations

MEDI-579 was formulated at 10 mg/mL in 25 mM histidine, 120 mM NaCl, pH6.0. The formulation had first Tm of 68° C.: pI range of 9.06 to 9.15. The formulation had good stability at 5° C. and -70° C. at 10, 50, 80 and 120 mg/mL over 22 months (Monomer decreased by 0.63% and 1.24% respectively at 50 mg/mL). However, this formulation had potentially prohibitively high viscosity at concentrations above 50 mg/mL.

All publications, patents and patent applications mentioned in this specification are herein incorporated by reference into the specification to the same extent as if each individual publication, patent or patent application was specifically and individually indicated to be incorporated herein by reference.
Foote J, Winter G. Antibody framework residues affecting the conformation of the hypervariable loops. J Mol. Biol. 1992; 224:487-99
Kotani I, Sato A, Hayakawa H, Urano T, Takada Y, Takada A. Increased procoagulant and antifibrinolytic activities in the lungs with idiopathic pulmonary fibrosis. Thromb Res. 1995; 77:493-504
Osada H, Yamada C, Miwa K, Kono T, Oh-hira M. An assay system for the modulators of plasminogen activation on the cell surface. Thromb Res. 1991; 62: 519-30
Osbourn J K, Field A, Wilton J, Derbyshire E, Earnshaw J C, Jones P T et al. Generation of a panel of related human scFv antibodies with high affinities for human CEA. Immunotechnology. 1996; 2:181-96
Persic L, Roberts A, Wilton J, Cattaneo A, Bradbury A, Hoogenboom H R. An integrated vector system for the eukaryotic expression of antibodies or their fragments after selection from phage display libraries. Gene. 1997; 187:9-18
Sidhu S S, Weiss G A. Constructing phage display libraries by oligonucleotide-directed mutagenesis. In: Clackson T, Lowman H B, editors. Phage Display: A Practical Approach. 1990 p 27-41.
Thompson J, Pope T, Tung J S, Chan C, Hollis G, Mark G et al. Affinity maturation of a high-affinity human monoclonal antibody against the third hypervariable loop of human immunodeficiency virus: use of phage display to improve affinity and broaden strain reactivity. J Mol. Biol. 1996; 256:77-88
Tomlinson I. VBASE. MRC Centre of Protein Engineering, Cambridge, UK. 1997
Vaughan T J, Williams A J, Pritchard K, Osbourn J K, Pope A R, Earnshaw J C, et al. Human antibodies with sub-nanomolar affinities isolated from a large non-immunized phage display library. Nat. Biotechnol. 1996; 14:309-14.
Sharp, A. M. et al., 1999 Structure, 7, 111-118.
Mottonen, J. et al., 1992 Nature, 355, 270-273.
Aertgeerts, K. et al., 1995 Nature Struct. Biol., 2, 891-897.
Declerck, P. J. et al., 1988 J Biol. Chem., 263, 15454-15461.
Seiffert, D. & Loskutoff, D. J. 1991 J Biol. Chem., 266, 2824-2830.
Elokdah, H. et al., 2004 Journal of Medicinal Chemistry, 47, 3491-3494. WO03/000253, 2003
Liang, A. et al., 2005 Thrombosis Research, 115, 341-350.
Charlton, P. A. et al., 1996 Thromb. Haemost., 75, 808-815.

TABLE 1
Coding polymorphisms in SERPINE1

| | | | Allele Frequency* | | | | |
|---|---|---|---|---|---|---|---|
| Position in | | | | African | | | Database |
| NM_000602 | SNP | Amino Acid Change | European | American | Chinese | Asian | Ref. |
| 118 | G-A | A15T | 11% | 0% | 12% | 15% | rs6092 |
| 124 | G-A | V17I | 2% | 10% | 4% | 0% | rs6090 |
| 149 | A-C | H25P | 0% | 2%** | No data | 0% | rs2227647 |
| 701 | G-A | R209H | 0% | 2% | No data | 0% | rs2227669 |
| 723 | C-T | Silent D216 | 0% | 2% | No data | No | rs2227670 |
| | | | | | | data | |
| 839 | C-A | T255N | 0% | 2% | No data | 0% | rs2227685 |

*Allele frequency data is based on 23 Caucasians and 24 African Americans sequenced by the Seattle SNP project. Chinese and Asian allele frequency data are taken from Mutation Catalogue and were estimated in populations of 42 and 83, respectively.
**A mutation catalogue entry for this SNP estimates the allele frequency in African Americans to be much higher at 24%.

TABLE 2
Antibodies 1 to 27 showing lineup of CDRs.
a.
b.
c.

| | HCDR1 | | | | | HCDR2 | | | | | | | | | | | | | | | | | HCDR3 | | | | |
|---|---|---|---|---|---|---|---|---|---|---|---|---|---|---|---|---|---|---|---|---|---|---|---|---|---|---|---|
| Kabat numbering | 31 | 32 | 33 | 34 | 35 | 50 | 51 | 52 | 52A | 53 | 54 | 55 | 56 | 57 | 58 | 59 | 60 | 61 | 62 | 63 | 64 | 65 | 95 | 96 | 97 | 98 | 99 |
| Antibody 01 | S | Y | A | I | S | G | I | I | P | I | F | G | T | A | N | Y | A | Q | K | F | Q | G | E | K | R | Q | W |
| Antibody 02 | | | | | | | | | | T | | G | | | | | | | | | | | | | | | |
| Antibody 03 | | | | | | | | | | T | | G | | | | | | | | | | | | | | | |
| Antibody 04 | | | | | | | | | | T | | G | | | | | | | | | R | | | | | | |
| Antibody 05 | | | | | | | | | | T | | S | | | | | | | | | R | | | | | | |
| Antibody 06 | | | | | | | | | | T | | G | | | | | | | | | | | | | | | |
| Antibody 07 | | | | | | | | | | T | | G | | | | | | | | | | | | | | | |
| Antibody 08 | | | | | | | | | | T | | G | | | | | | | | | | | | R | | | |
| Antibody 09 | | | | | | | | | | A | | | | | | | | | | | | | | | | | |
| Antibody 10 | | | | | | | | | | T | | G | | | | | | | | | | | | | | | |
| Antibody 11 | | | | | | | | | | | | G | | | | | | | | | | | | | | | |
| Antibody 12 | | | | | | | | | | T | | P | | | | | | | | | | | | | | | |
| Antibody 13 | | | | | | | | | | T | | G | | | | | | | | | | | | | | | |
| Antibody 14 | | | | | | | | | | T | | G | | | | | | | | | | | | | | | |
| Antibody 15 | | | | | | | | | | T | | G | | | | | | | | | | | | | | | |
| Antibody 16 | | | | | | | | | | T | | G | | | | | | | | | | | | | | | |
| Antibody 17 | | | | | | | | | | T | | G | | | | | | | | | | | | | | | |
| Antibody 18 | | | | | | | | | | T | | G | | | | | | | | L | | | | | | | |
| Antibody 19 | | | | | | | | | | | | | | | | | | | | | | | | R | | | |
| Antibody 20 | | | | | | | | | | T | | G | | | | | | | | | | | | | | | |
| Antibody 21 | | | | | | | | | | T | | G | | | | | | | | | | | | | | | |
| Antibody 22 | G | | | | | | | | | T | | G | | | | | S | | | | | | | | | | |
| Antibody 23 | | | | | | | | | | T | | G | | | | | | | | | | S | | | | | |
| Antibody 24 | | | | | | | | | | T | | V | | | | | | | | | | | | | | | |
| Antibody 25 | | | | | | | | | | T | | G | | | | | | | | | | | | R | | | |
| Antibody 26 | | | | | | | | | | | | G | | | | | | | | | | | | | | | |
| Antibody 27 | | | | | | | | | | T | | G | | | | | | | | | | | | | | | |

| | HCDR3 | | | | | | | LCDR1 | | | | | | | | | | | LCDR2 | | |
|---|---|---|---|---|---|---|---|---|---|---|---|---|---|---|---|---|---|---|---|---|---|
| Kabat numbering | 100 | 100A | 100B | 100C | 100D | 101 | 102 | 24 | 25 | 26 | 27 | 28 | 29 | 30 | 31 | 32 | 33 | 34 | 50 | 51 | 52 |
| Antibody 01 | L | E | G | H | F | D | Y | R | A | S | E | G | I | Y | R | S | L | A | K | A | S |
| Antibody 02 | | | | | | | | | | | | | | | | S | | | | | |
| Antibody 03 | | | | | | | | | | | | | | | R | S | | | | | |
| Antibody 04 | | | | R | | | | | | | | | | | | S | | | R | | T |
| Antibody 05 | | | | | | | | | | | | | | | | Q | | | R | | |
| Antibody 06 | | | | | | | | | | | | | | | | E | | | R | | |
| Antibody 07 | | | | | | | | | | | | | | | | E | | | R | | |
| Antibody 08 | | | | | | | | | | | | | | | | N | | | | | |
| Antibody 09 | | | | | | | | Q | | | | | | | | N | | | R | | |
| Antibody 10 | | | | | | | | | | | | | | | | S | | | R | | |
| Antibody 11 | | | | | | | | Q | | | | | | | | E | | | R | | |
| Antibody 12 | | | | | | | | | | | | | | | | N | | | R | | |
| Antibody 13 | | | | | | | | | | | | | | | | S | | | R | | |
| Antibody 14 | | | | | | | | | | | | | | | | N | | | R | | |
| Antibody 15 | | | | | | | | | | | | | | | | N | | | R | | |
| Antibody 16 | | | | | | | | | | | | S | | | | S | | | | | |
| Antibody 17 | | | | | | | | | | | | | | | | E | | | R | | |
| Antibody 18 | | | | | | | | | | | | | | | | A | | | R | | |
| Antibody 19 | | | | | | | | | | | | | | | | E | | | R | | |
| Antibody 20 | | | | | | | | | | | | | | | | E | | | R | | |
| Antibody 21 | | | | | | | | | | | | | | | | N | | | R | | |
| Antibody 22 | | | | | | | | | | | | | | | | A | | | R | | |
| Antibody 23 | | | | | | | | | | | | | | | | S | | | R | | |
| Antibody 24 | | | | | | | | | | | | | | | | A | | | R | | |
| Antibody 25 | | | | | | | | | | | | S | | | | E | | | | | |
| Antibody 26 | | | | | | | | | | | | | | | | T | | | R | | |
| Antibody 27 | | | | | | | | | | | | | | | | N | | | R | | |

| | LCDR2 | | | | LCDR3 | | | | | | | | |
|---|---|---|---|---|---|---|---|---|---|---|---|---|---|
| Kabat numbering | 53 | 54 | 55 | 56 | 89 | 90 | 91 | 92 | 93 | 94 | 95 | 96 | 97 |
| Antibody 01 | S | L | A | S | Q | Q | Y | S | N | Y | P | L | T |
| Antibody 02 | | | | | | | | | D | | | | |
| Antibody 03 | | | | | | | | | | | | | |
| Antibody 04 | | | | | | | | | | | | | |
| Antibody 05 | | | | | | | | | | | | | |
| Antibody 06 | | | | | | | | | | | | | |
| Antibody 07 | | | | | | | | | | | | | |
| Antibody 08 | | | | | | | | | | | | | |
| Antibody 09 | | | | | | | | | | | | | |
| Antibody 10 | | | | | | | | | | | | | |
| Antibody 11 | | | | | | | | | | | | | |
| Antibody 12 | | | | | | | | | | | | | |
| Antibody 13 | | | | | | | | | | | | | |
| Antibody 14 | | | | | | | | | | | | | |
| Antibody 15 | | | | | | | | | | | | | |
| Antibody 16 | | | | | | | | | | | | | |
| Antibody 17 | | | | | | | | | | | | | |
| Antibody 18 | | | | | | | | | | | | | |
| Antibody 19 | | | | | | | | | | | | | |
| Antibody 20 | | | | | | | | | D | | | | |
| Antibody 21 | | | | | | | | | | | | | |
| Antibody 22 | | | | | | | | | | | | | |
| Antibody 23 | | | | | | | | | | | | | |
| Antibody 24 | | | | | | | | | | | | | |
| Antibody 25 | | | | | | | | | | | | | |
| Antibody 26 | | | | | | | | | | | | | |
| Antibody 27 | | | | | | | | | | | | | |

TABLE 3
IC50 of antibodies 1 to 27 in chromogenic assay
d.

| | | | Chromogenic (IC50 in nM) | | | | | | |
|---|---|---|---|---|---|---|---|---|---|
| | | | | | cyno/ | | rat/ | | mouse/ |
| | format | GL | human | cyno | human | rat | human | mouse | human |
| Ab 01 | IgG1 | no | 1.5 | 0.7 | 0.4 | 99.3 | 66.2 | 30.0 | 20.0 |
| Ab 01 | IgG4 | no | Inc | 0.4 | N/A | Inc | N/A | 13.6 | N/A |
| Ab 01 | IgG4 | yes | 1.6 | N/D | N/A | 114.5 | 71.6 | N/D | N/A |
| Ab 01 | scFv | no | 3.4 | 1.2 | 0.4 | 208.0 | 60.5 | 28.3 | 8.2 |
| Ab 02 | IgG1 | no | 1.6 | N/D | N/A | 510.0 | 316.8 | 167.0 | 103.7 |
| Ab 02 | IgG4 | no | Inc | 0.4 | N/A | Inc | N/A | Inc | N/A |
| Ab 02 | IgG4 | yes | 1.4 | 1.0 | 0.7 | 545.0 | 389.3 | 150.0 | 107.1 |
| Ab 02 | scFv | no | 0.6 | 1.1 | 1.8 | 530.0 | 890.8 | 553.0 | 929.4 |
| Ab 03 | IgG4 | no | Inc | 0.5 | N/A | Inc | N/A | 12.9 | N/A |
| Ab 03 | scFv | no | 1.8 | 0.1 | 0.1 | 297.0 | 165.0 | 54.9 | 30.5 |
| Ab 04 | scFv | no | 6.6 | 2.1 | 0.3 | 340.0 | 51.4 | 264.0 | 39.9 |
| Ab 05 | scFv | no | 5.0 | 0.5 | 0.1 | 860.0 | 172.3 | 718.0 | 143.9 |
| Ab 06 | scFv | no | 74.6 | 31.6 | 0.4 | 37.1 | 0.5 | 48.2 | 0.6 |
| Ab 07 | scFv | no | 187.0 | 117.0 | 0.6 | 91.0 | 0.5 | 143.0 | 0.8 |
| Ab 08 | IgG1 | no | 1.9 | 0.8 | 0.4 | 2.0 | 1.1 | 1.2 | 0.6 |
| Ab 08 | scFv | no | 3.2 | 1.0 | 0.3 | 3.0 | N/A | Inc | N/A |
| Ab 09 | scFv | no | Inc | 0.7 | N/A | 29.7 | N/A | Inc | N/A |
| Ab 10 | scFv | no | 2.2 | 0.5 | 0.2 | 296.0 | 132.1 | 334.0 | 149.1 |
| Ab 11 | scFv | no | 102.0 | 43.8 | 0.4 | 59.5 | 0.6 | 33.6 | 0.3 |
| Ab 12 | scFv | no | 2.3 | 0.6 | 0.3 | 78.2 | 34.4 | 27.8 | 12.2 |
| Ab 13 | scFv | no | 2.9 | 1.2 | 0.4 | 140.0 | 48.6 | 247.0 | 85.8 |
| Ab 14 | scFv | no | Inc | 0.8 | N/A | 23.1 | N/A | Inc | N/A |
| Ab 15 | scFv | no | 7.2 | 2.2 | 0.3 | 64.2 | 8.9 | 66.9 | 9.3 |
| Ab 16 | IgG1 | no | 1.6 | 0.5 | 0.3 | 2.7 | 1.7 | 2.0 | 1.3 |
| Ab 16 | scFv | no | Inc | Inc | N/A | 5.6 | N/A | 4.3 | N/A |
| Ab 17 | scFv | no | 71.5 | 41.5 | 0.6 | 24.9 | 0.3 | 26.2 | 0.4 |
| Ab 18 | scFv | no | 1.3 | 0.5 | 0.4 | 238.0 | 190.4 | 319.0 | 255.2 |
| Ab 19 | scFv | no | 26.0 | 10.8 | 0.4 | 7.9 | 0.3 | 6.4 | 0.2 |
| Ab 20 | IgG1 | no | 1.6 | 0.8 | 0.5 | 2.9 | 1.8 | 1.4 | 0.9 |
| Ab 20 | scFv | no | Inc | 1.3 | N/A | Inc | N/A | 4.9 | N/A |
| Ab 21 | scFv | no | 4.1 | 1.6 | 0.4 | 49.2 | 12.0 | 32.2 | 7.9 |
| Ab 22 | scFv | no | 5.0 | 1.5 | 0.3 | 406.0 | 81.2 | 413.0 | 82.6 |
| Ab 23 | scFv | no | 1.6 | 0.5 | 0.3 | 376.0 | 229.3 | 376.0 | 229.3 |
| Ab 24 | scFv | no | 3.2 | 1.1 | 0.3 | 127.0 | 39.3 | 140.0 | 43.3 |
| Ab 25 | scFv | no | 13.1 | 4.6 | 0.4 | 322.0 | 24.6 | 172.0 | 13.1 |
| Ab 26 | scFv | no | 4.0 | 1.2 | 0.3 | 245.0 | 61.4 | 211.0 | 52.9 |
| Ab 27 | IgG1 | no | 4.9 | N/D | N/A | 26.5 | 5.4 | 16.1 | 3.3 |
| Ab 27 | scFv | no | Inc | 2.2 | N/A | 49.8 | N/A | Inc | N/A |

Ab = Antibody
GL = germlined
Inc = incomplete inhibition curve
N/D = not determined
N/A = not applicable

TABLE 4
IC50 (nM) of selected antibodies in chromogenic assay
with CHO cell-produced glycosylated human PAI-1

| | | | Chromogenic (IC50 in nM) |
|---|---|---|---|
| | format | GL | hu glyco |
| Antibody 01 | IgG1 | no | 1.7 |
| Antibody 01 | IgG4 | yes | 1.6 |
| Antibody 02 | IgG1 | no | 1.5 |
| Antibody 02 | IgG4 | yes | 1.5 |
| Antibody 08 | IgG1 | no | 1.3 |
| Antibody 08 | IgG1 | yes | 1.7 |
| Antibody 16 | IgG1 | no | 1.2 |
| Antibody 16 | IgG1 | yes | 1.4 |
| Antibody 20 | IgG1 | no | 1.3 |
| Antibody 20 | IgG1 | yes | 2.0 |
| Antibody 27 | IgG1 | no | 2.6 |

GL = germlined

TABLE 5
IC50 (nM) of selected antibodies in HT-1080 plasminogen
activation assay with exogenous human, mouse and rat PAI-1

| | | | HT-1080 Exo PAI-1 | | |
|---|---|---|---|---|---|
| | | | (IC50 in nM) | | |
| | format | GL | human | mouse | rat |
| Antibody 01 | IgG1 | no | 11.2 | 15.8 | Inc |
| Antibody 01 | IgG4 | no | 8.0 | 14.3 | Inc |
| Antibody 01 | IgG4 | yes | 17.0 | N/D | N/D |
| Antibody 01 | scFv | no | 23.9 | 34.5 | Inc |
| Antibody 02 | IgG1 | no | 8.7 | 10.9 | Inc |
| Antibody 02 | IgG4 | no | 8.4 | 9.7 | Inc |
| Antibody 02 | IgG4 | yes | 9.4 | N/D | N/D |
| Antibody 03 | IgG4 | no | 9.5 | Inc | Inc |
| Antibody 08 | IgG1 | no | 10.5 | 4.5 | 17.7 |
| Antibody 08 | IgG1 | yes | 11.1 | N/D | 18.5 |
| Antibody 08 | scFv | no | 23.7 | 26.2 | 50.1 |
| Antibody 09 | scFv | no | 23.0 | 21.0 | 52.7 |
| Antibody 14 | scFv | no | 28.0 | 22.0 | 52.0 |
| Antibody 15 | scFv | no | 36.0 | 37.0 | 57.0 |
| Antibody 16 | IgG1 | no | 7.4 | 5.7 | 17.3 |
| Antibody 16 | IgG1 | yes | 11.3 | N/D | 19.6 |
| Antibody 16 | scFv | no | 16.9 | 17.6 | 52.6 |
| Antibody 18 | scFv | no | N/D | N/D | 37.0 |
| Antibody 20 | IgG1 | no | 7.3 | 5.9 | 16.2 |
| Antibody 20 | scFv | no | 19.1 | 18.1 | 42.4 |
| Antibody 27 | IgG1 | no | 10.9 | 7.8 | 16.1 |
| Antibody 27 | scFv | no | 18.8 | 16.4 | 33.8 |

GL = germlined
Inc = incomplete inhibition curve
N/D = not determined

TABLE 6a
Binding kinetic and affinity data for Antibody 08 for human,
cyno and rat PAI-1 as determined by BIAcore at 25° C.

| | BIAcore data | | | |
|---|---|---|---|---|
| | On rate | Off rate | Dissociation | |
| PAI-1 | (Ka) | (Kd) | constant (K_{D}) | |
| protein | (1/MS) | (1/s) | (pM) | n value |
| Human | 7.02 × 10⁶ | 4.19 × 10⁻⁵ | 6 | n = 5 |
| glycosylated | | | | |
| PAI-1 | | | | |
| Human stable | 5.90 × 10⁶ | 1.17 × 10⁻⁴ | 19 | n = 2 |
| mutant PAI-1 | | | | |
| Cyno PAI-1 | 3.56 × 10⁶ | 4.75 × 10⁻⁵ | 13 | n = 5 |
| Rat PAI-1 | 7.64 × 10⁶ | 7.98 × 10⁻⁴ | 105 | n = 2 |

TABLE 6b
Binding kinetic and affinity data for Antibody 08 for human,
cyno and rat PAI-1 as determined by BIAcore at 37° C.
e.
f.

| | BIAcore data | | | |
|---|---|---|---|---|
| | On rate | Off rate | Dissociation | |
| PAI-1 | (Ka) | (Kd) | constant (K_{D}) | |
| protein | (1/MS) | (1/s) | (pM) | n value |
| Human | 6.37 × 10⁶ | 4.43 × 10⁻⁵ | 7 | n = 2 |
| glycosylated | | | | |
| PAI-1 | | | | |
| Human stable | 7.23 × 10⁶ | 2.84 × 10⁻⁴ | 39 | n = 2 |
| mutant PAI-1 | | | | |
| Cyno PAI-1 | 4.90 × 10⁶ | 6.34 × 10⁻⁵ | 13 | n = 1 |

TABLE 7
Ratios of IC50 values of selected antibodies to glycosylated active human
PAI-1, latent human PAI-1, rat PAI-1, mouse PAI-1 and rabbit PAI-1 in
competition with non-glycosylated active human PAI-1 (“wt”)

| | | | Competition ELISA | | | | |
|---|---|---|---|---|---|---|---|
| | | | hu | | | | |
| | | | glyco/ | latent/ | rat/ | mouse/ | rabbit/ |
| | format | GL | wt | wt | wt | wt | wt |
| Antibody 01 | IgG4 | no | N/D | 8.8 | 331.0 | 98.0 | 9.0 |
| Antibody 02 | IgG4 | no | N/D | 6.4 | 33.0 | 48.0 | 4.8 |
| Antibody 08 | IgG1 | no | 1.2 | 8.5 | 9.7 | 47.0 | 41.5 |
| Antibody 08 | IgG1 | yes | 1.6 | 20.1 | 11.5 | 72.9 | N/D |
| Antibody 16 | IgG1 | no | 1.7 | 11.2 | 4.8 | 14.4 | 7.9 |
| Antibody 16 | IgG1 | yes | 2.1 | 15.1 | 9.8 | 70.0 | 15.4 |
| Antibody 20 | IgG1 | no | N/D | 7.5 | 2.3 | 7.6 | 9.1 |
| Antibody 27 | IgG1 | no | N/D | 6.8 | 1.2 | 6.1 | 85.0 |

GL = germlined
N/D = not determined

TABLE 8
Top 10 binding peptides in PEPSCAN

| SEQ ID NO | Peptide sequence | Detection signal |
|---|---|---|
| 125 | CGHYYDIC* | 1366 |
| 126 | CGHYYDCGHYDC* | 1094 |
| 127 | CGHYYDGCGGHYDGC* | 764 |
| 128 | CGHYYDCNYTEFC* | 707 |
| 129 | CGHYYDILC* | 702 |
| 130 | CDGHYYDIC* | 642 |
| 131 | CNYTEFCGHYYDC* | 598 |
| 132 | CRNVVFSPYGCASVLAMLQLC | 584 |
| 125 | CGHYYDIC* | 532 |
| 133 | CVVFSPYGVACVLAMLQLTTC | 531 |

TABLE 9
g. Signal/background ratios for PEPSCAN peptides

| SEQ | | Ratio (Signal/average |
|---|---|---|
| ID NO | Peptide sequence | background signal) |
| 125 | CGHYYDIC | 9.97 |
| 134 | CGHYYDCGHYYDC | 7.98 |
| 135 | CGHYYDGCGGHYYDGC | 5.58 |
| 128 | CGHYYDCNYTEFC | 5.16 |
| 129 | CGHYYDILC | 5.12 |
| 130 | CDGHYYDIC | 4.69 |
| 131 | CNYTEFCGHYYDC | 4.36 |
| 136 | CRNVVFSPYGCASVLAMQLC | 4.26 |
| 125 | CGHYYDIC | 3.88 |
| 133 | CVVFSPYGVACVLAMLQLTTC | 3.87 |
| 137 | CHYYDILELC | 3.70 |
| 138 | CYYDILELC | 3.66 |
| 139 | CYYDILEC | 3.63 |
| 140 | CFNYTEFTTCGHYYDILELC | 3.50 |
| 141 | CHYYDILC | 3.45 |
| 142 | CGAVDQLTRLCLVNALYFNGC | 3.41 |
| 143 | CQTNKFNYCTRLPRLLC* | 3.36 |
| 137 | CHYYDILELC | 3.31 |
| 137 | CHYYDILELC | 3.28 |
| 129 | CGHYYDILC | 3.26 |
| 138 | CYYDILELC | 3.24 |
| 144 | CHYYDILEC | 3.23 |
| 145 | CEFTTCGHYYDILEC | 3.23 |
| 146 | CLFVVRHNPTCTVLFMGQVMC | 3.22 |
| 147 | CGHYYDILEC | 3.19 |
| 148 | CQTNKFNYCTRLPRLC | 3.18 |
| 149 | CNVVFSPYGVCSVLAMLQLTC | 3.13 |
| 150 | CGHYYDILELC | 3.08 |
| 151 | CTNKFNYCTRLPRLC | 3.07 |
| 152 | CFTTPDGHYYDILC | 3.04 |
| 153 | CQTNKFNYTECNMTRLPC | 3.04 |
| 154 | CFNYTEFTTPCGHYYDILELC | 3.02 |
| 155 | CTNKFNYCTRLPRLLVC | 3.00 |
| 130 | CDGHYYDIC | 2.99 |

TABLE 10
PAI-1/Antibody 08 Fab direct interactions - table of
epitope and paratope residues

| PAI-1 | | |
|---|---|---|
| (SEQ ID | | |
| NO: 124) | Heavy chain | Light Chain |
| Tyr 210 | | Tyr 30, Water 5, Water 15 |
| Thr 211 | | Tyr 30 |
| Glu 212 | | Tyr 30, His 31, Arg 66, Water 5 |
| Tyr 220 | | His 31, Arg 66, Ser 67, Gly 68 |
| Asp 222 | | Tyr 30, Water 5 |
| Tyr 241 | | Lys 50, His 31, Water 5 |
| Leu 269 | | Tyr 30, Ser 92, Asn 93 |
| Pro 270 | | Ser 92, Asn 93, Tyr 94 |
| Arg 271 | | Tyr 30, Asn 32, Tyr 91, Ser 92, |
| | | Ser 92, Asn 93, Water 15 |
| Leu 272 | Trp 99(103), Leu 100 (104) | |
| Arg 346 | Phe 54 (55) | |
| Met 347 | Gly 50, Ile 51, Ile 52, | Tyr 94 |
| | Phe 54(55), Thr 56(57), | |
| | Ala 57(58), Asn | |
| | 58(59), Leu 100(104), | |
| | Glu 100A(105) | |
| Ala 348 | Trp 99(103), | Tyr 94 |
| | Leu 100(104), | |
| | Glu 100A(105), | |
| | Gly 100B(106) | |
| Pro 349 | Glu 95(99), Arg 97(101), | Tyr 91, Ser 92, Asn 93, Tyr 94, |
| | Leu 100(104), | Leu 96 |
| | Glu 100A(105), | |
| | Gly 100B(106) | |
| Glu 350 | Arg 97(101), Leu 100(104) | Asn 32, Lys 50, Tyr 91, |
| | | Water 15 |
| Glu 351 | Arg 97(101), Leu 100(104) | Lys 50 |

Amino acid residues of the heavy and light chain of Antibody 08 are numbered according to Kabat. Where the numbering of the heavy chain residues assigned from X-ray crystal structure analysis differs, the numbering is given in parentheses.
Interactions within 3.2 A are in bold.
Interactions with the main-chain are underlined.

TABLE 11
Crystal Parameters and X-ray Data-Processing and Refinement Statistics

| | Native I | Native II |
|---|---|---|
| Data collection | ID23 ESRF | ID29 ESRF |
| Space group | P2₁ | P2₁ |
| Cell dimensions | | |
| a, b, c (Å) | 90.1 | 90.2 |
| | 89.7 | 89.6 |
| | 250.8 | 249.7 |
| a, b, g (°) | 90.0 | 90.0 |
| | 99.8 | 99.3 |
| | 90.0 | 90.0 |
| Osc./# frameas | 0.3/600 | 0.2/1100 |
| Wavelength | 1.072 | 0.976 |
| Resolution (Å) | 63-3.24 (3.47-3.24) | 20-2.9 (3.06-2.9) |
| R_{sym} or R_{merge} | 0.156 (0.46) | 0.095 (0.47) |
| I/sI | 3.1 (2.3) | 4.4 (2.1) |
| Completeness (%) | 99.9 (99.7) | 94.4 (78.6) |
| Redundancy | 3.6 (2.6) | 4.8 (1.6) |
| Refinement | | |
| Resolution (Å) | 3.2 | 2.9 |
| No. reflections | 11387 | 82314 |
| R_{work/} R_{free} | 0.23/0.27 | 0.25/0.30 |
| No. atoms | 24768 | 24796 |
| Protein | 24768 | 4024 |
| Ligand/ion | — | — |
| Water | — | 100 |
| B-factors | 71 | 60 |
| Protein | — | — |
| Ligand/ion | — | — |
| Water | — | — |
| R.m.s deviations | | |
| Bond lengths | 0.003 | 0.006 |
| (Å) | | |
| Bond angles (°) | 0.574 | 1.1 |

TABLE 12
PAI-1 epitope comparisons with Antibody 08, MAI-12 and H4B3

| PAI-1/Antibody 08 | | |
|---|---|---|
| epitope | MAI-12 [208] | H4B3 [208] |
| Tyr 210 | x | ✓ |
| Thr 211 | x | x |
| Glu 212 | x | ✓ |
| Tyr 220 | ✓ | x |
| Asp 222 | x | x |
| Tyr 241 | ✓ | ✓ |
| Leu 269 | x | x |
| Pro 270 | x | x |
| Arg 271 | ✓ | ✓ |
| Leu 272 | x | x |
| Arg 346 | x | x |
| Met 347 | x | x |
| Ala 348 | x | x |
| Pro 349 | x | x |
| Glu 350 | x | x |
| Glu 351 | x | x |

✓ present from the PAI-1/Antibody 08 epitope
x absent from the PAI-1/Antibody 08 epitope

TABLE 13
PAI-1 epitope comparisons of Antibody 08 with scFv-56A7C10

| Active PAI-1/scFv- | |
|---|---|
| 56A7C10 [220] | Antibody 08 |
| Arg 187 | x |
| His 190 | x |
| Tyr 210 | ✓ |
| Thr 211 | ✓ |
| Glu 212 | ✓ |
| Phe 213 | x |
| Thr 214 | x |
| Thr 215 | x |
| Pro 216 | x |
| Asp 217 | x |
| Gly 218 | x |
| His 219 | x |
| Tyr 220 | ✓ |
| Asp 222 | ✓ |
| Tyr 241 | ✓ |
| Glu 242 | x |
| Lys 243 | x |
| Glu 244 | x |
| Gly 264 | x |
| Asn 265 | x |
| Met 266 | x |
| Thr 267 | x |
| Leu 269 | ✓ |
| Pro 270 | ✓ |
| Arg 271 | ✓ |
| Met 347 | ✓ |
| Pro 349 | ✓ |
| Glu 350 | ✓ |
| Glu 351 | ✓ |
| Ile 352 | x |
| Ile 353 | x |
| Asp 355 | x |

✓ present from the PAI-1/scFv-56A7C10 epitope
x absent from the PAI-1/scFv-56A7C10 epitope

TABLE 14
Epitope mapping showing residues present in the
PAI-1/candidate antibody epitope (Antibody 08)

| PAI-1 | BIAcore data | | | |
|---|---|---|---|---|
| mutant | On rate | Off rate | Dissociation | |
| protein | (1/MS) | (1/s) | constant (K_{D}) | Effect of mutation |
| Stable | 2.87e6 | 6.95e−5 | 0.027 nM | n/a |
| mutant, | | | | |
| wild-type | | | | |
| Y220S | 1.73e6 | 1.04e−4 | 0.39 nM | Interferes with |
| | | | | interactions to LC |
| | | | | H31, R66, S67 and |
| | | | | G68 |
| Y241S | 1.99e6 | 2.92e−4 | 0.15 nM | Interferes with |
| | | | | interactions to LC |
| | | | | K50 and H31 |
| E212V | 1.03e6 | 1.26e−3 | 1.23 nM | Breaks salt bridge |
| | | | | between E212 and |
| | | | | LC R66 |
| R271S | 1.37e6 | 1.01e−2 | 7.4 nM - | Breaks H-bond |
| | | | negligible binding | network between |
| | | | | LC S92, N32, Water |
| | | | | 15, PAI-1 R271 |
| Y220S | 4.45e6 | 6.02e−4 | 0.14 nM | |
| T267K | | | | |
| Y210N | 6.94e6 | 3.6e−4 | 0.05 nM | Interferes with |
| | | | | interactions to LC |
| | | | | Y30 |

LC = light chain

TABLE 15
Primer sequences for generation of PAI-1 mutant
proteins as described in Example 11
(SEQ ID NOs: 202-212)

| Primer | Sequence |
|---|---|
| HPAI1Nde | 5'CTCTCATATGGTTCACCATCCCCCA TCCTA 3' |
| HPAI1XhoI | 5'ACACCTCGAGTCAGGGTTCCATCAC TTG 3' |
| N150HK154T | 5'GGTATGATCAGCCACTTGCTTGGGA CAGGAGC |
| | CGTGGACC 3' |
| Q319L | 5'CTCCACGTCGCGCTGGCGCTGCAGA AAG 3' |
| M354I | 5'CCGAGGAGATCATCATCGACAGACC CTTCC 3' |
| Y220S | 5'ACGCCCGATGGCCATTCCTACGACA TCCTGGA 3' |
| Y241S | 5'TCATTGCTGCCCCTTCTGAAAAAGA GGTGCC 3' |
| E212V | 5'AGTTCAACTATACTGTGTTCACCAC GCCCGA 3' |
| R271S | 5'ATGACCAGGCTGCCCAGCCTCCTGG TTCTGC 3' |
| T267K | 5'GGAAAGGCAACATGAAAAGGCTGCC CCGCCT 3' |
| Y210N | 5'ACCAACAAGTTCAACAATACTGAGT TCACC 3' |

TABLE 16
List of Antibody Sequences

| SEQ ID NO: | Description |
|---|---|
| SEQ ID NO: 5 | Antibody 01, VH DNA |
| SEQ ID NO: 6 | Antibody 01, VH PRT |
| SEQ ID NO: 7 | Antibody 01, VL DNA |
| SEQ ID NO: 8 | Antibody 01, VL PRT |
| SEQ ID NO: 9 | Antibody 02, VH DNA |
| SEQ ID NO: 10 | Antibody 02, VH PRT |
| SEQ ID NO: 11 | Antibody 02, VL DNA |
| SEQ ID NO: 12 | Antibody 02, VL PRT |
| SEQ ID NO: 13 | Antibody 03, VH DNA |
| SEQ ID NO: 14 | Antibody 03, VH PRT |
| SEQ ID NO: 15 | Antibody 03, VL DNA |
| SEQ ID NO: 16 | Antibody 03, VL PRT |
| SEQ ID NO: 17 | Antibody 04, VH DNA |
| SEQ ID NO: 18 | Antibody 04, VH PRT |
| SEQ ID NO: 19 | Antibody 04, VL DNA |
| SEQ ID NO: 20 | Antibody 04, VL PRT |
| SEQ ID NO: 21 | Antibody 05, VH DNA |
| SEQ ID NO: 22 | Antibody 05, VH PRT |
| SEQ ID NO: 23 | Antibody 05, VL DNA |
| SEQ ID NO: 24 | Antibody 05, VL PRT |
| SEQ ID NO: 25 | Antibody 06, VH DNA |
| SEQ ID NO: 26 | Antibody 06, VH PRT |
| SEQ ID NO: 27 | Antibody 06, VL DNA |
| SEQ ID NO: 28 | Antibody 06, VL PRT |
| SEQ ID NO: 29 | Antibody 07, VH DNA |
| SEQ ID NO: 30 | Antibody 07, VH PRT |
| SEQ ID NO: 31 | Antibody 07, VL DNA |
| SEQ ID NO: 32 | Antibody 07, VL PRT |
| SEQ ID NO: 33 | Antibody 08, VH DNA |
| SEQ ID NO: 34 | Antibody 08, VH PRT |
| SEQ ID NO: 35 | Antibody 08, VL DNA |
| SEQ ID NO: 36 | Antibody 08, VL PRT |
| SEQ ID NO: 37 | Antibody 09, VH DNA |
| SEQ ID NO: 38 | Antibody 09, VH PRT |
| SEQ ID NO: 39 | Antibody 09, VL DNA |
| SEQ ID NO: 40 | Antibody 09, VL PRT |
| SEQ ID NO: 41 | Antibody 10, VH DNA |
| SEQ ID NO: 42 | Antibody 10, VH PRT |
| SEQ ID NO: 43 | Antibody 10, VL DNA |
| SEQ ID NO: 44 | Antibody 10, VL PRT |
| SEQ ID NO: 45 | Antibody 11, VH DNA |
| SEQ ID NO: 46 | Antibody 11, VH PRT |
| SEQ ID NO: 47 | Antibody 11, VL DNA |
| SEQ ID NO: 48 | Antibody 11, VL PRT |
| SEQ ID NO: 49 | Antibody 12, VH DNA |
| SEQ ID NO: 50 | Antibody 12, VH PRT |
| SEQ ID NO: 51 | Antibody 12, VL DNA |
| SEQ ID NO: 52 | Antibody 12, VL PRT |
| SEQ ID NO: 53 | Antibody 13, VH DNA |
| SEQ ID NO: 54 | Antibody 13, VH PRT |
| SEQ ID NO: 55 | Antibody 13, VL DNA |
| SEQ ID NO: 56 | Antibody 13, VL PRT |
| SEQ ID NO: 57 | Antibody 14, VH DNA |
| SEQ ID NO: 58 | Antibody 14, VH PRT |
| SEQ ID NO: 59 | Antibody 14, VL DNA |
| SEQ ID NO: 60 | Antibody 14, VL PRT |
| SEQ ID NO: 61 | Antibody 15, VH DNA |
| SEQ ID NO: 62 | Antibody 15, VH PRT |
| SEQ ID NO: 63 | Antibody 15, VL DNA |
| SEQ ID NO: 64 | Antibody 15, VL PRT |
| SEQ ID NO: 65 | Antibody 16, VH DNA |
| SEQ ID NO: 66 | Antibody 16, VH PRT |
| SEQ ID NO: 67 | Antibody 16, VL DNA |
| SEQ ID NO: 68 | Antibody 16, VL PRT |
| SEQ ID NO: 69 | Antibody 17, VH DNA |
| SEQ ID NO: 70 | Antibody 17, VH PRT |
| SEQ ID NO: 71 | Antibody 17, VL DNA |
| SEQ ID NO: 72 | Antibody 17, VL PRT |
| SEQ ID NO: 73 | Antibody 18, VH DNA |
| SEQ ID NO: 74 | Antibody 18, VH PRT |
| SEQ ID NO: 75 | Antibody 18, VL DNA |
| SEQ ID NO: 76 | Antibody 18, VL PRT |
| SEQ ID NO: 77 | Antibody 19, VH DNA |
| SEQ ID NO: 78 | Antibody 19, VH PRT |
| SEQ ID NO: 79 | Antibody 19, VL DNA |
| SEQ ID NO: 80 | Antibody 19, VL PRT |
| SEQ ID NO: 81 | Antibody 20, VH DNA |
| SEQ ID NO: 82 | Antibody 20, VH PRT |
| SEQ ID NO: 83 | Antibody 20, VL DNA |
| SEQ ID NO: 84 | Antibody 20, VL PRT |
| SEQ ID NO: 85 | Antibody 21, VH DNA |
| SEQ ID NO: 86 | Antibody 21, VH PRT |
| SEQ ID NO: 87 | Antibody 21, VL DNA |
| SEQ ID NO: 88 | Antibody 21, VL PRT |
| SEQ ID NO: 89 | Antibody 22, VH DNA |
| SEQ ID NO: 90 | Antibody 22, VH PRT |
| SEQ ID NO: 91 | Antibody 22, VL DNA |
| SEQ ID NO: 92 | Antibody 22, VL PRT |
| SEQ ID NO: 93 | Antibody 23, VH DNA |
| SEQ ID NO: 94 | Antibody 23, VH PRT |
| SEQ ID NO: 95 | Antibody 23, VL DNA |
| SEQ ID NO: 96 | Antibody 23, VL PRT |
| SEQ ID NO: 97 | Antibody 24, VH DNA |
| SEQ ID NO: 98 | Antibody 24, VH PRT |
| SEQ ID NO: 99 | Antibody 24, VL DNA |
| SEQ ID NO: 100 | Antibody 24, VL PRT |
| SEQ ID NO: 101 | Antibody 25, VH DNA |
| SEQ ID NO: 102 | Antibody 25, VH PRT |
| SEQ ID NO: 103 | Antibody 25, VL DNA |
| SEQ ID NO: 104 | Antibody 25, VL PRT |
| SEQ ID NO: 105 | Antibody 26, VH DNA |
| SEQ ID NO: 106 | Antibody 26, VH PRT |
| SEQ ID NO: 107 | Antibody 26, VL DNA |
| SEQ ID NO: 108 | Antibody 26, VL PRT |
| SEQ ID NO: 109 | Antibody 27, VH DNA |
| SEQ ID NO: 110 | Antibody 27, VH PRT |
| SEQ ID NO: 111 | Antibody 27, VL DNA |
| SEQ ID NO: 112 | Antibody 27, VL PRT |
| SEQ ID NO: 113 | Antibodies 1-27 VH CDR 1 |
| | PRT |
| SEQ ID NO: 114 | Antibody 19 VH CDR2 PRT |
| SEQ ID NO: 115 | Antibodies 1-3, 5, 6, 7, 10, 12-18, |
| | 20-24, 26 or 27 VH CDR3 |
| | PRT |
| SEQ ID NO: 117 | Antibody 01, 02, 03, 08, 16 or |
| | 25 VL CDR2 PRT |
| SEQ ID NO: 118 | Antibody 01, 3-19, 21-27 VL |
| | CDR 3 PRT |
| SEQ ID NO: 119 | PAI-1 human PRT |
| SEQ ID NO: 120 | PAI-1 rat PRT |
| SEQ ID NO: 121 | PAI-1 mouse PRT |
| SEQ ID NO: 122 | PAI-1 cyno PRT |
| SEQ ID NO: 123 | PAI-1 canine PRT |
| SEQ ID NO: 124 | PAI-1 mature human PRT |
| SEQ ID NOs: 125-155 | synthetic peptides of |
| | peptide-scanning assay |
| SEQ ID NOs: 156-158 | human PAI-1 epitopes |
| SEQ ID NO: 159 | Antibody 22, VH CDR1 PRT |
| SEQ ID NO: 160 | Antibody 01, 11 or 26, VH |
| | CDR2 PRT |
| SEQ ID NO: 161 | Antibody 02, 03, 06, 08, 10, |
| | 13, 17, 20, 21, 25 or 27, VH |
| | CDR2 PRT |
| SEQ ID NO: 162 | Antibody 04, VH CDR2 PRT |
| SEQ ID NO: 163 | Antibody 05, VH CDR2 PRT |
| SEQ ID NO: 164 | Antibody 09, VH CDR2 PRT |
| SEQ ID NO: 165 | Antibody 12, VH CDR2 PRT |
| SEQ ID NO: 166 | Antibody 18, VH CDR2 PRT |
| SEQ ID NO: 167 | Antibody 22, VH CDR2 PRT |
| SEQ ID NO: 168 | Antibody 23, VH CDR2 PRT |
| SEQ ID NO: 169 | Antibody 24, VH CDR2 PRT |
| SEQ ID NO: 170 | Antibody 04, VH CDR3 PRT |
| SEQ ID NO: 171 | Antibody 08, 19 or 25, VH |
| | CDR3 PRT |
| SEQ ID NO: 172 | Antibody 01 or 03, VL CDR1 |
| | PRT |
| SEQ ID NO: 173 | Antibody 02, 04, 10, 13 or 23, |
| | VL CDR1 PRT |
| SEQ ID NO: 174 | Antibody 05, VL CDR1 PRT |
| SEQ ID NO: 175 | Antibody 06, 07, 17, 19 or 20, |
| | VL CDR1 PRT |
| SEQ ID NO: 176 | Antibody 08, 12, 14, 15, 21 or |
| | 27, VL CDR1 PRT |
| SEQ ID NO: 177 | Antibody 09, VL CDR1 PRT |
| SEQ ID NO: 178 | Antibody 11, VL CDR1 PRT |
| SEQ ID NO: 179 | Antibody 16, VL CDR1 PRT |
| SEQ ID NO: 180 | Antibody 18, 22 or 24, VL |
| | CDR1 PRT |
| SEQ ID NO: 181 | Antibody 25, VL CDR1 PRT |
| SEQ ID NO: 182 | Antibody 26, VL CDR1 PRT |
| SEQ ID NO: 183 | Antibody 04, VL CDR2 PRT |
| SEQ ID NO: 184 | Antibody 05, 07, 09, 15, 17, |
| | 24, 26 or 27, VL CDR2 PRT |
| SEQ ID NO: 185 | Antibody 02 or 20, VL CDR3 |
| | PRT |
| SEQ ID NO: 186 | Antibody 01, 03, 04-18, 21-27, |
| | VL CDR3 PRT |
| SEQ ID NO: 187 | Antibodies 1-27, VH CDR1 |
| | PRT |
| SEQ ID NO: 188 | mutated PAI-1 epitope |
| SEQ ID NO: 189 | R271S human PAI-1 |
| SEQ ID NO: 190 | stable mutant human PAI-1 |
| SEQ ID NO: 191 | R292S stable mutant human |
| | PAI-1 |
| SEQ ID NO: 192 | human PAI-1 epitope |
| SEQ ID NO: 193 | human PAI-1 epitope |
| SEQ ID NO: 194 | Y210N stable mutant human |
| | PAI-1 |
| SEQ ID NO: 195 | E212V stable mutant human |
| | PAI-1 |
| SEQ ID NO: 196 | Y220S stable mutant human |
| | PAI-1 |
| SEQ ID NO: 197 | Y241S stable mutant human |
| | PAI-1 |
| SEQ ID NO: 198 | R271S stable mutant human |
| | PAI-1 |
| SEQ ID NO: 199 | Y220 T267K stable mutant |
| | human PAI-1 |
| SEQ ID NO: 200 | cloned HIS-tagged wild type |
| | PAI-1 |
| SEQ ID NO: 201 | HIS tagged stable PAI-1 |
| SEQ ID NOS: 202-212 | primer sequences |
| SEQ ID NO: 213 | HCDR2 paratope |
| SEQ ID NO: 214 | HCDR3 paratope |
| SEQ ID NO: 215 | LCDR3 paratope |

TABLE 17
Key lead isolation reagents

| | Expression | | |
|---|---|---|---|
| Reagent | System | Source | Use |
| Human PAI-1 | E. coli | Calbiochem | Selections, phage |
| | | | ELISA and |
| | | | biochemical assays |
| Rat PAI-1 | E. coli | Calbiochem | Selections and phage |
| | | | ELISA |
| tPA (Actilyse) | | Boehringer | Biochemical assays |
| | | Ingelheim | |
| AD380 (anti-PAI-1 | | American | Biochemical assays |
| mAb) | | Diagnostica | |
| Anti-human tPA | | Biopol | Biochemical assays |

TABLE 18
Key lead optimisation reagents

| | Expression | | |
|---|---|---|---|
| Reagent | System | Source | Use |
| Human PAI-1 | E. coli | Molecular | Selections, |
| (wild-type) | | Innovations | biochemical assays, |
| | | | cross-reactivity |
| | | | assays |
| Human PAI-1 | Mammalian | AZ | Biochemical assays |
| (wild-type) | (CHO) | | |
| Human PAI-1 | E. coli | Progen | Selections |
| (mutant) | | | |
| Rat PAI-1 | E. coli | US Biological | Selections, cross- |
| | | | reactivity assays |
| Cynomolgus PAI-1 | E. coli | CAT | Cross-reactivity |
| | | | assays, biochemical |
| | | | and in vitro assays |
| tPA (Actilyse) | | Boehringer | Biochemical assays |
| | | Ingelheim | |

i Sharp, A. M. et al., 1999 Structure, 7, 111-118.
ii Mottonen, J. et al., 1992 Nature, 355, 270-273.
iii Aertgeerts, K. et al., 1995 Nature Struct. Biol., 2, 891-897.
iv Declerck, P. J. et al., 1988 J Biol. Chem., 263, 15454-15461.
v Seiffert, D. & Loskutoff, D. J. 1991 J Biol. Chem., 266, 2824-2830.
vi Deng, G. et al., 1996 J. Cell Biol., 134, 1563-1571.
vii Elokdah, H. et al., 2004 Journal of Medicinal Chemistry, 47, 3491-3494.
viii Wyeth WO03/000253, 2003
ix Liang, A. et al., 2005 Thrombosis Research, 115, 341-350.
x Charlton, P. A. et al., 1996 Thromb. Haemost., 75, 808-815.

We claim:

1. A method of treating a disease or condition in a subject in need thereof, wherein the disease or condition is selected from one or more of: systemic lupus erythromatosus (SLE), scleroderma, pulmonary fibrosis, diabetic nephropathy, lupus nephritis, graft versus host disease, glomerulonephritis, focal segmental glomerulosclerosis, membranous nephropathy, mesangial proliferative glomerulonephritis, membranoproliferative glomerulonephritis, renal fibrosis, and COPD, comprising administering to said subject an effective amount of a human or chimeric antibody or antibody fragment, wherein the antibody or antibody fragment immunospecifically binds to human PAI-1 and inhibits PAI-1 activity.

2. The method of claim 1, wherein the antibody or antibody fragment immunospecifically binds to a human PAI-1:vitronectin complex, but does not disrupt the binding of human PAI-1 to vitronectin, and wherein the antibody or antibody fragment can stimulate plasmin-mediated activation of MMP-1.

3. The method of claim 1 or 2, wherein the antibody or antibody fragment is administered as a composition comprising a purified antibody or antibody fragment and a pharmaceutically acceptable carrier.

4. The method of claim 3, wherein the composition is a pyrogen-free composition.

5. The method of any of claims 1-4, wherein the antibody or antibody fragment is a human antibody or antibody fragment.

6. The method of any of claims 1-5, wherein the antibody or antibody fragment is a monoclonal antibody

7. The method of any of claims 1-6, wherein the antibody or antibody fragment has two or more of the following characteristics:
(a) affinity (K_{D}) between 5 pm to 200 pM for active human PAI-1, as assessed by surface plasmon resonance;
(b) immunospecifically binds to a human PAI-1:vitronectin complex, but does not disrupt the binding of human PAI-1 to vitronectin;
(c) immunospecifically binds to glycosylated human PAI-1;
(d) does not immunospecifically bind to human PAI-2 or human PAI-3
(e) inhibits the binding of human PAI-1 to tPA by at least 50%;
(f) inhibits the binding of human PAI-1 to tPA with an IC₅₀ of about 5 nM or less;
(g) inhibits the binding of human PAI-1 to uPA by at least 50%;
(h) immunospecifically binds human PAI-1, mouse PAI-1, and rat PAI-1;
(i) immunospecifically binds human PAI-1, mouse PAI-1, and PAI-1 from at least one non-human primate;
(j) immunospecifically binds cynomolgus PAI-1;
(k) immunospecifically binds to a human PAI-1 epitope chosen from SEQ ID NOs: 156-158;
(l) specifically binds to the reactive center loop of PAI-1 that interacts with tPA and uPA
(m) binds to the same epitope as Antibody 8;
(n) preferentially binds to active human PAI-1 over latent human PAI-1;
(o) can reduce the level of VCAM-1 in dermal tissues;
(p) can reduce the level of TNF-alpha in dermal tissues;
(q) can stimulate plasmin-mediated activation of MMP-1.

8. The method of claim 7, wherein the antibody or antibody fragment has at least three of characteristics (a)-(q).

9. The method of claim 8, wherein the antibody or antibody fragment has at least four of characteristics (a)-(q).

10. The method of claim 9, wherein the antibody or antibody fragment has at least five of characteristics (a)-(q).

11. The method of claim 10, wherein the antibody or antibody fragment has at least six of characteristics (a)-(q).

12. The method of claim 11, wherein the antibody or antibody fragment has at least eight of characteristics (a)-(q).

13. The method of claim 12, wherein the antibody or antibody fragment has at least nine of characteristics (a)-(q).

14. The method of claim 13, wherein the antibody or antibody fragment has at least ten of characteristics (a)-(q).

15. The method of claim 14, wherein the antibody or antibody fragment has at least twelve of characteristics (a)-(q).

16. The method of claim 15, wherein the antibody or antibody fragment has at least 14 of characteristics (a)-(q).

17. The method of claim 16, wherein the antibody or antibody fragment has at least sixteen of characteristics (a)-(q).

18. The method of any of claims 1-17, wherein the antibody or antibody fragment immunospecifically binds cynomolgus PAI-1 with approximately the same affinity as human PAI-1.

19. The method of any of claims 1-18, wherein the antibody or antibody fragment is one or more of: a monoclonal antibody; a human antibody; a chimeric antibody; a single-chain Fv (scFv); an Fab fragment; an F(ab′) fragment; an intrabody; and a synthetic antibody.

20. The method of claim 19, wherein the antibody or antibody fragment is a human antibody.

21. The method of claim 19, wherein the antibody or antibody fragment is a chimeric antibody.

22. The method of any of claims 1-21, wherein the antibody or antibody fragment is conjugated to a detectable substance or a therapeutic agent.

23. The method of any of claims 1-22, wherein the disease or condition is SLE.

24. The method of any of claims 1-23, wherein the disease or condition is scleroderma.

25. The method of claim 24, wherein treating comprises treating digital ulcers associated with scleroderma.

26. The method of any of claims 1-25, wherein the disease or condition is lupus nephritis.

27. The method of any of claims 1-26, wherein the disease or condition is diabetic nephropathy.

28. The method of any of claims 1-27, wherein said subject is human.

29. The method of any of claims 1-29, wherein the antibody or antibody fragment is MEDI-579

30. A purified or isolated polypeptide, comprising the amino acid sequence of SEQ ID NO: A.

31. The polypeptide of claim 30, wherein said polypeptide is glycosylated.

32. The polypeptide of claim 30 or 31, wherein said polypeptide is glycosylated in a manner that differs from the native glycosylation pattern of the naturally occurring polypeptide.

33. A purified or isolated polypeptide, comprising the amino acid sequence of SEQ ID NO: B or SEQ ID NO: C, wherein said polypeptide is glycosylated.

34. The polypeptide of claim 33, comprising the amino acid sequence of SEQ ID NO: B.

35. The polypeptide of claim 33, comprising the amino acid sequence of SEQ ID NO: C.

36. The polypeptide of claim 33, consisting of the amino acid sequence of SEQ ID NO: B or SEQ ID NO: C.

37. The polypeptide of any of claims 33-36, wherein said polypeptide is glycosylated in a manner that differs from the native glycosylation pattern of the naturally occurring polypeptide.

38. A purified or isolated complex, comprising a polypeptide having the amino acid sequence of SEQ ID NO:A and a vitronectin polypeptide.

39. The complex of claim 38, wherein said vitronectin polypeptide is a dog or human vitronectin polypeptide

40. A purified or isolated complex, comprising a polypeptide having the amino acid sequence of SEQ ID NO:B and a vitronectin polypeptide.

41. A purified or isolated complex, comprising a polypeptide having the amino acid sequence of SEQ ID NO:C and a vitronectin polypeptide.

42. The complex of claim 40 or 41, wherein the vitronectin polypeptide comprises a cynomolgous or human vitronectin polypeptide.


SEQUENCE LISTING (256 sequences)

SEQ ID NO: 1-4 - skipped (4 entries)

SEQ ID NO: 5 - DNA, 363 bp, Homo sapiens
  misc_feature (1)..(363): Antibody 01
caggtacagctggtgcagtcaggggctgaggtgaagaagcctggctcttcggtgaaggtctcctgcaaggcttctggaggcaccttcagcagctatgctatcagctgggtgcgacaggcccctggacaagggcttgagtggatgggagggatcatccctatttttggtacagcaaactacgcacagaagttccagggcagagtcacgattaccgcggacgaatccacgagcacagcctacatggagctgagcagcctgagatctgaggacacggccgtctattactgtgcgagagagaagaggcagtggctggaaggacactttgactactggggccggggcaccctggtcaccgtctcctca

SEQ ID NO: 6 - protein, 121 aa, Homo sapiens
  misc_feature (1)..(121): Antibody 01
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCAREKRQWLEGHFDYWGRGTLVTVSS

SEQ ID NO: 7 - DNA, 321 bp, Homo sapiens
  misc_feature (1)..(321): Antibody 01
gacatccagatgacccagtctccatccaccctgtctgcatctgttggagacagagtcaccatcacctgccgggccagtgagggtatttatcgctcgttggcctggtatcagcagaagccagggaaagcccctaaactcctgatctataaggcctctagtttagccagtggggtcccatcaaggttcagcggcagtggatctgggacagaattcactctcaccatcagcagcctgcagcctgatgattttgcaacttattactgccaacaatatagtaattatccgctcactttcggcggagggaccaaggtggagatcaaa

SEQ ID NO: 8 - protein, 107 aa, Homo sapiens
  misc_feature (1)..(107): Antibody 01
DIQMTQSPSTLSASVGDRVTITCRASEGIYRSLAWYQQKPGKAPKLLIYKASSLASGVPSRFSGSGSGTEFTLTISSLQPDDFATYYCQQYSNYPLTFGGGTKVEIK

SEQ ID NO: 9 - DNA, 363 bp, Homo sapiens
  misc_feature (1)..(363): Antibody 02
caggtacagctggtgcagtcaggggctgaggtgaagaagcctggctcttcggtgaaggtctcctgcaaggcttctggaggcaccttcagcagctatgctatcagctgggtgcgacaggcccctggacaagggcttgagtggatgggagggatcatccctacttttggtacagcaaactacgcacagaagttccagggcagagtcacgattaccgcggacgaatccacgagcacagcctacatggagctgagcagcctgagatctgaggacacggccgtctattactgtgcgagagagaagaggcagtggctggaaggacactttgactactggggccggggcaccctggtcaccgtctcctca

SEQ ID NO: 10 - protein, 121 aa, Homo sapiens
  misc_feature (1)..(121): Antibody 02
QVQLVQSGAEVKKPGSSVKVSCKASGGTFSSYAISWVRQAPGQGLEWMGGIIPTFGTANYAQKFQGRVTITADESTSTAYMELSSLRSEDTAVYYCAREKRQWLEGHFDYWGRGTLVTVSS

SEQ ID NO: 11 - DNA, 321 bp, Homo sapiens
  misc_feature (1)..(321): Antibody 02
gacatccagatgacccagtctccatccaccctgtctgcatctgttggagacagagtcaccatcacctgccgggccagtgagggtatttatcactcgttggcctggtatcagcagaagccagggaaagcccctaaactcctgatctataaggcctctagtttagccagtggggtcccatcaaggttcagcggcagtggatctgggacagaattcactctcaccatcagcagcctgcagcctgatgattttgcaacttattactgccaacaatatagtgattatccgctcactttcggcggagggaccaaggtggagatcaaa

SEQ ID NO: 12 - protein, 107 aa, Homo sapiens
  misc_feature (1)..(107): Antibody 02
DIQMTQSPSTLSASVGDRVTITCRASEGIYHSLAWYQQKPGKAPKLLIYKASSLASGVPSRFSGSGSGTEFTLTISSLQPDDFATYYCQQYSDYPLTFGGGTKVEIK

SEQ ID NO: 13 - DNA, 363 bp, Homo sapiens
  misc_feature (1)..(363): Antibody 03
caggtacagctgcagcagtcaggggctgaggtgaagaagcctggctcttcggtgaaggtctcctgcaaggcttctggaggcaccttcagcagctacgctatcagctgggtgcgacaggcccctggacaagggcttgagtggatgggagggatcatccctacttttggtacagcaaactatgcacagaagttccagggcagagtcacgattaccgcggacgaatccacgagcacagcctacatggagctgagcggcctgaggtctgaggacacagccgtctattactgtgcgagagagaagaggcagtggctggaaggacactttgactactggggccggggcaccccggtcaccgtctcctca

SEQ ID NO: 14 - protein, 121 aa, Homo sapiens
  misc_feature (1)..(121): Antibody 03
QVQLQQSGAEVKKPGSSVKVSCKASGGTFSSYAISWVRQAPGQGLEWMGGIIPTFGTANYAQKFQGRVTITADESTSTAYMELSGLRSEDTAVYYCAREKRQWLEGHFDYWGRGTPVTVSS

SEQ ID NO: 15 - DNA, 321 bp, Homo sapiens
  misc_feature (1)..(321): Antibody 03
gacatccagatgacccagtccccatccttcctgtctgcatctattggagacagagtcaccatcacctgccgggccagtgagggtatttatcgctccttggcctggtatcagcagaagccagggaaagccccaaaactcctgatctataaggcctctagtttagccagtggggccccatcaaggttcagcggcagtggatctgggacagatttcactctcaccatcagcggcctgcagcctgatgattttgcgacttattactgccaacaatatagtaattatccgctcactttcggcggagggaccaagctggagatcaaa

SEQ ID NO: 16 - protein, 107 aa, Homo sapiens
  misc_feature (1)..(107): Antibody 03
DIQMTQSPSFLSASIGDRVTITCRASEGIYRSLAWYQQKPGKAPKLLIYKASSLASGAPSRFSGSGSGTDFTLTISGLQPDDFATYYCQQYSNYPLTFGGGTKLEIK

SEQ ID NO: 17 - DNA, 363 bp, Homo sapiens
  misc_feature (1)..(363): Antibody 04
caggtacggctgcagcagtcaggggctgaggtgaagaagcctggctcttcggtgaaggtctcctgcaaggcttctggaggcaccttcagcagctacgctatcagctgggtgcgacaggcccctggacaagggcttgagtggatgggagggatcatccctacttttggtacagcaaactacgcacagaagttccggggcagagtcacgattaccgcgaacgaatccacgagcacagcctacatggagctgagcggcctgaggtctgaggacacggccgtctactactgtgcgagagagaagaggcagtggctggaaggacgctttgactactggggccggggcaccctggtcaccgtctcgagc

SEQ ID NO: 18 - protein, 121 aa, Homo sapiens
  misc_feature (1)..(121): Antibody 04
QVRLQQSGAEVKKPGSSVKVSCKASGGTFSSYAISWVRQAPGQGLEWMGGIIPTFGTANYAQKFRGRVTITANESTSTAYMELSGLRSEDTAVYYCAREKRQWLEGRFDYWGRGTLVTVSS

SEQ ID NO: 19 - DNA, 321 bp, Homo sapiens
  misc_feature (1)..(321): Antibody 04
gacatccagatgacccagtctccatccttcctgtctgcatctattggagacagaatcaccatcacctgccgggccagtgagggtatttatcactcgttggcctggtatcagcagaagccagggaaagcccctaaactcctgatccatagggccactagtttagccagtgggaccccatcaaggttcggcggcagtggatctgggacggatttcgctctcaccatcagcagcctgcagcctgatgattttgcaacttattactgccaacaatatagtaactatccgctcactttcggcggagggaccaagctggagatcaaa

SEQ ID NO: 20 - protein, 107 aa, Homo sapiens
  misc_feature (1)..(107): Antibody 04
DIQMTQSPSFLSASIGDRITITCRASEGIYHSLAWYQQKPGKAPKLLIHRATSLASGTPSRFGGSGSGTDFALTISSLQPDDFATYYCQQYSNYPLTFGGGTKLEIK

SEQ ID NO: 21 - DNA, 363 bp, Homo sapiens
  misc_feature (1)..(363): Antibody 05
caggtacagctgcagcagtcaggggctgaggtgaagaagcctggctcttcggtgaaggtctcctgcaaggcttctggaggcaccttcagcagctacgctatcagctgggtgcgacaggcccctgggcaagggcttgagtggatgggagggatcatccctactttttctacagcaaactacgcacagaagttccggggcagagtcacgattaccgcggacgaatccacgagcacagcctacatggagctgagcggcctgaggtctgaggacacggccgtctattactgtgcgagagagaagaggcagtggctggaaggacactttgactactggggccggggcaccccggtcaccgtctcgagt

SEQ ID NO: 22 - protein, 121 aa, Homo sapiens
  misc_feature (1)..(121): Antibody 05
QVQLQQSGAEVKKPGSSVKVSCKASGGTFSSYAISWVRQAPGQGLEWMGGIIPTFSTANYAQKFRGRVTITADESTSTAYMELSGLRSEDTAVYYCAREKRQWLEGHFDYWGRGTPVTVSS

SEQ ID NO: 23 - DNA, 321 bp, Homo sapiens
  misc_feature (1)..(321): Antibody 05
gacatccagatgacccagtccccatccttcctgtctgcatctattggagacagagtcaccatcacctgccgggccagtgagggtatttatcaccagttggcctggtatcagcagaagccagggaaagccccaaaactcctgatctatagggcctctagtttagccagtggggccccatcaaggttcagcggcagtggatctgggacagatttcactctcaccatcagcagcctgcagcctgatgattttgcgacttattactgccaacaatatagtaattatccgctcactttcggcggagggaccaagctggagatcaaa

SEQ ID NO: 24 - protein, 107 aa, Homo sapiens
  misc_feature (1)..(107): Antibody 05
DIQMTQSPSFLSASIGDRVTITCRASEGIYHQLAWYQQKPGKAPKLLIYRASSLASGAPSRFSGSGSGTDFTLTISSLQPDDFATYYCQQYSNYPLTFGGGTKLEIK

SEQ ID NO: 25 - DNA, 363 bp, Homo sapiens
  misc_feature (1)..(363): Antibody 06
caggtacagctgcagcagtcaggggctgaggtgaagaagcctggctcttcggtgaaggtctcctgcaaggcttctggaggcaccttcagcagctacgctatcagctgggtgcgacaggcccctggacaagggcttgagtggatgggagggatcatccctacttttggtacagcaaactacgcacagaagttccagggcagagtcacgattaccgcggacgaatccacgagcacagcctacatggagctgagcggcctgaggtctgaggacacggccgtctattactgtgcgagagagaagaggcagtggctggaaggacactttgactactggggccggggcaccccggtcaccgtctcgagt

SEQ ID NO: 26 - protein, 121 aa, Homo sapiens; identical to SEQ ID NO: 14
  misc_feature (1)..(121): Antibody 06

SEQ ID NO: 27 - DNA, 321 bp, Homo sapiens
  misc_feature (1)..(321): Antibody 06
gacatccagatgacccagtccccatccttcctgtctgcatctattggagacagagttaccatcacctgccgggccagtgagggtatttatcacgagttggcctggtatcagcagaagccagggaaagccccaaaactcctgatctatagggcctctagtttagccagtggggccccatcaaggttcagcggcagtggatctgggacagatttcactctcaccatcagcagcctgcagcctgatgattttgcgacttattactgccaacaatatagtaattatccgctcactttcggcagagggaccaagctggagatcaag

SEQ ID NO: 28 - protein, 107 aa, Homo sapiens
  misc_feature (1)..(107): Antibody 06
DIQMTQSPSFLSASIGDRVTITCRASEGIYHELAWYQQKPGKAPKLLIYRASSLASGAPSRFSGSGSGTDFTLTISSLQPDDFATYYCQQYSNYPLTFGRGTKLEIK

SEQ ID NO: 29 - DNA, 363 bp, Homo sapiens
  misc_feature (1)..(363): Antibody 07
caggtacagctgcagcagtcaggggctgaggtgaagaagcctggctcttcggtgaaggtctcctgcaaggcttctggaggcaccttcagcagctacgctatcagctgggtgcgacaggcccctggacaagggcttgagtgggtgggagggatcatccctacttttggcacagcaaactacgcacagaagttccagggcagagtcacgattaccgcggacgaatccacgagcacagcctacatggagctgagcggcctgaggtctgaggacacggccgtctattactgtgcgagagagaagaggcagtggctggaaggacactttgactactggggccggggcaccccggtcaccgtctcgagt

SEQ ID NO: 30 - protein, 121 aa, Homo sapiens
  misc_feature (1)..(121): Antibody 07
QVQLQQSGAEVKKPGSSVKVSCKASGGTFSSYAISWVRQAPGQGLEWVGGIIPTFGTANYAQKFQGRVTITADESTSTAYMELSGLRSEDTAVYYCAREKRQWLEGHFDYWGRGTPVTVSS

SEQ ID NO: 31 - DNA, 321 bp, Homo sapiens
  misc_feature (1)..(321): Antibody 07
gacatccagatggcccagtccccatccttcctgtctgcatctattggagacagagtcaccatcacctgccgggccagtgagggtatttatcacgagttggcctggtatcagcagaagccagggaaagccccaaaactcctgatctatagggcctctagtttagccagtggggccccatcaaggttcagcggcagtggatccgggacagatttcactctcaccatcagcagcctgcagcctgatgattttgcgacttattactgccaacaatatagtaattatccgctcactttcggcggagggaccaagctggagatcaaa

SEQ ID NO: 32 - protein, 107 aa, Homo sapiens
  misc_feature (1)..(107): Antibody 07
DIQMAQSPSFLSASIGDRVTITCRASEGIYHELAWYQQKPGKAPKLLIYRASSLASGAPSRFSGSGSGTDFTLTISSLQPDDFATYYCQQYSNYPLTFGGGTKLEIK

SEQ ID NO: 33 - DNA, 363 bp, Homo sapiens
  misc_feature (1)..(363): Antibody 08
caggtacagctggtgcagtcaggggctgaggtgaagaagcctggctcttcggtgaaggtctcctgcaaggcttctggaggcaccttcagcagctacgctatcagctgggtgcgacaggcccctggacaagggcttgagtggatgggagggatcatccctacttttggtacagcaaactacgctcagaagttccagggcagagtcacgattaccgcggacgaatccacgagcacagcctacatggagctgagcagcctgaggtctgaggacacggccgtctattactgtgcgagagagaggaggcagtggctggaaggacactttgactactggggccggggcaccctggtcaccgtctcctca

SEQ ID NO: 34 - protein, 121 aa, Homo sapiens
  misc_feature (1)..(121): Antibody 08
QVQLVQSGAEVKKPGSSVKVSCKASGGTFSSYAISWVRQAPGQGLEWMGGIIPTFGTANYAQKFQGRVTITADESTSTAYMELSSLRSEDTAVYYCARERRQWLEGHFDYWGRGTLVTVSS

SEQ ID NO: 35 - DNA, 321 bp, Homo sapiens
  misc_feature (1)..(321): Antibody 08
gacatccagatgacccagtccccatccaccctgtctgcatctgttggagacagagtcaccatcacctgccgggccagtgagggtatttatcacaacttggcctggtatcagcagaagccagggaaagccccaaaactcctgatctataaggcctctagtttagccagtggggtcccatcaaggttcagcggcagtagatctgggacagagttcactctcaccatcagcagcctgcagcctgatgattttgcgacttattactgccaacaatatagtaattatccacttactttcggcggagggaccaaggtggagatcaaa

SEQ ID NO: 36 - protein, 107 aa, Homo sapiens
  misc_feature (1)..(107): Antibody 08
DIQMTQSPSTLSASVGDRVTITCRASEGIYHNLAWYQQKPGKAPKLLIYKASSLASGVPSRFSGSRSGTEFTLTISSLQPDDFATYYCQQYSNYPLTFGGGTKVEIK

SEQ ID NO: 37 - DNA, 363 bp, Homo sapiens
  misc_feature (1)..(363): Antibody 09
caggtacagctgcagcagtcaggggctgaggtgaagaagcctggctcttcggtgaaggtctcctgcaaggcttctggaggcaccttcagcagctacgctatcagctgggtgcgacaggcccctggacaagggcttgagtggatgggagggatcatccctgcttttgacacagcaaactacgcacaaaagttccagggcagagtcacgattaccgcggacgaatccacgagcacagcctacatggagctgagcggcctgaggtctgaggacacggccgtctattactgtgcgagagagaagaggcagtggctggaaggacactttgactactggggccggggcaccccggtcaccgtctcgagt

SEQ ID NO: 38 - protein, 121 aa, Homo sapiens
  misc_feature (1)..(121): Antibody 09
QVQLQQSGAEVKKPGSSVKVSCKASGGTFSSYAISWVRQAPGQGLEWMGGIIPAFDTANYAQKFQGRVTITADESTSTAYMELSGLRSEDTAVYYCAREKRQWLEGHFDYWGRGTPVTVSS

SEQ ID NO: 39 - DNA, 321 bp, Homo sapiens
  misc_feature (1)..(321): Antibody 09
gacatccagatgacccagtccccatccttcctgtctgcatctattggagacagagtcaccatcacctgccaggccagtgagggtatttatcacaacttggcctggtatcagcagaagccagggaaagccccaaaactcctgatctatagggcctctagtttagccagtggggccccatcaaggttcagcggcagtggatctgggacagatttcactctcaccatcagcagcctgcagcctgatgattttgcgacttattactgccaacaatatagtaattatccgctcactttcggcggagggaccaagttggagatcaaa

SEQ ID NO: 40 - protein, 107 aa, Homo sapiens
  misc_feature (1)..(107): Antibody 09
DIQMTQSPSFLSASIGDRVTITCQASEGIYHNLAWYQQKPGKAPKLLIYRASSLASGAPSRFSGSGSGTDFTLTISSLQPDDFATYYCQQYSNYPLTFGGGTKLEIK

SEQ ID NO: 41 - DNA, 363 bp, Homo sapiens
  misc_feature (1)..(363): Antibody 10
caggtacagctgcagcagtcaggggctgaggtgaagaagcctggctcttcggtgaaggtctcctgcaaggcttctggaggcaccttcagcagctacgctatcagctgggtgcgacaggcccctggacaagggcttgagtggatgggagggatcatccctacttttggtacagcaaactacgcacagaagttccagggcagagtcacgattaccgcggacgagtccacgagcacagcctacatggagttgagcagcttgagatctgaggacacggccgtctattactgtgcgagagagaagaggcagtggctggaaggacactttgactactggggccggggcaccccggtcaccgtctcgagt

SEQ ID NO: 42 - protein, 121 aa, Homo sapiens
  misc_feature (1)..(121): Antibody 10
QVQLQQSGAEVKKPGSSVKVSCKASGGTFSSYAISWVRQAPGQGLEWMGGIIPTFGTANYAQKFQGRVTITADESTSTAYMELSSLRSEDTAVYYCAREKRQWLEGHFDYWGRGTPVTVSS

SEQ ID NO: 43 - DNA, 321 bp, Homo sapiens
  misc_feature (1)..(321): Antibody 10
gacatccagatgacccagtccccatccttcctgtctgcatctattggagacagggtcgccatcacctgccgggccagtgagggtatttatcactccttggcctggtatcagcagaagccagggaaagccccaaaactcctgatctatagggcctctagtttagccagtggggccccatcaaggttcagcggcagtggatctgggacagatttcactctcaccatcagcagcctgcagcctgatgattttgcgacttattactgccaacaatatagtaattatccgctcactttcggcggagggaccaggctggagatcaaa

SEQ ID NO: 44 - protein, 107 aa, Homo sapiens
  misc_feature (1)..(107): Antibody 10
DIQMTQSPSFLSASIGDRVAITCRASEGIYHSLAWYQQKPGKAPKLLIYRASSLASGAPSRFSGSGSGTDFTLTISSLQPDDFATYYCQQYSNYPLTFGGGTRLEIK

SEQ ID NO: 45 - DNA, 363 bp, Homo sapiens
  misc_feature (1)..(363): Antibody 11
caggtacagctgcagcagtcaggggctgaggtgaagaagcctggctcttcggtgaaggtctcctgcaaggcttctggaggcaccttcagcagctacgctatcagctggatgcgacaggcccctggacaagggcttgagtggatgggagggatcatccctatctttggtacagcaaactacgcacagaagttccagggcagagtcacgattaccgcggacgaatccacgagcacagcctacatggagctgagcggcctgaggtctgaggacacggccgtctattactgtgcgagagagaagaggcagtggctggaaggacactttgactactggggccggggcaccccggtcactgtttcgagt

SEQ ID NO: 46 - protein, 121 aa, Homo sapiens
  misc_feature (1)..(121): Antibody 11
QVQLQQSGAEVKKPGSSVKVSCKASGGTFSSYAISWMRQAPGQGLEWMGGIIPIFGTANYAQKFQGRVTITADESTSTAYMELSGLRSEDTAVYYCAREKRQWLEGHFDYWGRGTPVTVSS

SEQ ID NO: 47 - DNA, 321 bp, Homo sapiens
  misc_feature (1)..(321): Antibody 11
gacatccagatgatccagtccccatccttcctgtctgcatctactggagacagagtcaccatcacctgccaggccagtgagggtatttatcacgagttggcctggtatcagcagaagccagggaaagccccaaaactcctgatctatagggcctctagtttagccagtggggccccatcaaggttcagcggcagtggatctgggacagatttcactctcaccatcagcagcctgcagcctgatgattttgcgacttattactgccaacaatatagtaattatccgctcactttcggcggagggaccaagctggagatcaaa

SEQ ID NO: 48 - protein, 107 aa, Homo sapiens
  misc_feature (1)..(107): Antibody 11
DIQMIQSPSFLSASTGDRVTITCQASEGIYHELAWYQQKPGKAPKLLIYRASSLASGAPSRFSGSGSGTDFTLTISSLQPDDFATYYCQQYSNYPLTFGGGTKLEIK

SEQ ID NO: 49 - DNA, 363 bp, Homo sapiens
  misc_feature (1)..(363): Antibody 12
caggtacagctgcagcagtcaggggctgaggtgaagaagcctggctcttcggtgaaggtctcctgcaaggcttctgggggcaccttcagcagctatgctatcagctgggtgcgacaggcccctggacaagggcttgagtggatgggagggatcatccctacttttcctacagcaaactacgcacagaagttccagggcagagtcacgattaccgcggacgaatccacgagcacagcctacatggagctgagcggcctgaggtctgaggacacggccgtctattactgtgcgagagagaagaggcagtggctggaaggacactttgactactggggccggggcaccccggtcaccgtctcgagt

SEQ ID NO: 50 - protein, 121 aa, Homo sapiens
  misc_feature (1)..(121): Antibody 12
QVQLQQSGAEVKKPGSSVKVSCKASGGTFSSYAISWVRQAPGQGLEWMGGIIPTFPTANYAQKFQGRVTITADESTSTAYMELSGLRSEDTAVYYCAREKRQWLEGHFDYWGRGTPVTVSS

SEQ ID NO: 51 - DNA, 321 bp, Homo sapiens
  misc_feature (1)..(321): Antibody 12
gacatccagatgacccagtccccatccttcctgtctgcatctattggagacagagtcaccatcacctgccgggccagtgagggtatttatcacaacttggcctggtatcaacagaagccagggaaagcccctaaactcctgatctatagggcctctagtttagccagtggggccccatcaaggttcagcggcagcggatctgggacagatttcactctcaccatcagcagcctgcagcctgatgattttgcaacttattactgccaacaatatagtaattatccgctcactttcggcggagggaccaagctggagatcaaa

SEQ ID NO: 52 - protein, 107 aa, Homo sapiens
  misc_feature (1)..(107): Antibody 12
DIQMTQSPSFLSASIGDRVTITCRASEGIYHNLAWYQQKPGKAPKLLIYRASSLASGAPSRFSGSGSGTDFTLTISSLQPDDFATYYCQQYSNYPLTFGGGTKLEIK

SEQ ID NO: 53 - DNA, 363 bp, Homo sapiens
  misc_feature (1)..(363): Antibody 13
caggtacagctgcagcagtcaggggctgaggtgaagaagcctggctcttcggtgaaggtctcctgcaaggcttctggaggcaccttcagcagctacgctatcagctgggtgcgacaggcccctggacaagggcttgagtggatgggagggatcatccctacttttggtacagcaaactacgcacagaagttccagggcagagtcacgattaccgcggacgaatccacgagcacagcctacatggagctgagcgacctgaggtctgaggacacggccgtctattactgtgcgagagagaagaggcagtggctggaaggacactttgactactggggccggggcaccccggtcaccgtctcgagt

SEQ ID NO: 54 - protein, 121 aa, Homo sapiens
  misc_feature (1)..(121): Antibody 13
QVQLQQSGAEVKKPGSSVKVSCKASGGTFSSYAISWVRQAPGQGLEWMGGIIPTFGTANYAQKFQGRVTITADESTSTAYMELSDLRSEDTAVYYCAREKRQWLEGHFDYWGRGTPVTVSS

SEQ ID NO: 55 - DNA, 321 bp, Homo sapiens
  misc_feature (1)..(321): Antibody 13
gacatccaaatgacccagtccccatccttcctgtctgcatctattggagacagagtcaccatcacctgccgggccagtgagggtatttatcactcgttggcctggtatcagcagaagccagggaaagccccaaaacttctgatctatagggcctctagtttagccagtggggccccatcaaggttcagcggcagtggatctgggacagatttcactctcaccatcagcagcctgcagcctgatgattttgcaacttattactgccaacaatatagtaattatccgctcactttcggcggagggaccaagctggagatcaaa

SEQ ID NO: 56 - protein, 107 aa, Homo sapiens
  misc_feature (1)..(107): Antibody 13
DIQMTQSPSFLSASIGDRVTITCRASEGIYHSLAWYQQKPGKAPKLLIYRASSLASGAPSRFSGSGSGTDFTLTISSLQPDDFATYYCQQYSNYPLTFGGGTKLEIK

SEQ ID NO: 57 - DNA, 363 bp, Homo sapiens
  misc_feature (1)..(363): Antibody 14
caggtacagctgcaacagtcaggggctgaggtgaagaagcctggctcttcggtgaaggtctcctgcaaggcttctggaggcaccttcagcagctacgctatcagctgggtgcgacaggcccccggacaagggcttgagtggatgggagggatcatccctacttttggtacagcaaactacgcacagaagttccagggcagagtcacgattaccgcggacgaatccacgagcacagcctacatggagctgagcggcctgaggtctgaggacacggccgtctattactgtgcgagagagaagaggcagtggctggaaggacactttgactactggggccggggcaccctggtcaccgtctcgagt

SEQ ID NO: 58 - protein, 121 aa, Homo sapiens
  misc_feature (1)..(121): Antibody 14
QVQLQQSGAEVKKPGSSVKVSCKASGGTFSSYAISWVRQAPGQGLEWMGGIIPTFGTANYAQKFQGRVTITADESTSTAYMELSGLRSEDTAVYYCAREKRQWLEGHFDYWGRGTLVTVSS

SEQ ID NO: 59 - DNA, 321 bp, Homo sapiens
  misc_feature (1)..(321): Antibody 14
gacatccagatgacccagtccccatccttcctgtctgcatctattggggacagagtcaccatcacctgccgggccagtgagggtatttatcacaacttggcctggtatcagcagaagccagggaaagccccaaaactcctgatctatagggcctctagtttagccagtggggccccatcaaggttcagcggcagtggatctgggacagatttcactttcaccatcagcagcctgcagcctgatgattttgcgacttattactgccaacaatatagtaattatccgctcactttcggcggagggaccaagctggagatcaaa

SEQ ID NO: 60 - protein, 107 aa, Homo sapiens
  misc_feature (1)..(107): Antibody 14
DIQMTQSPSFLSASIGDRVTITCRASEGIYHNLAWYQQKPGKAPKLLIYRASSLASGAPSRFSGSGSGTDFTFTISSLQPDDFATYYCQQYSNYPLTFGGGTKLEIK

SEQ ID NO: 61 - DNA, 363 bp, Homo sapiens
  misc_feature (1)..(363): Antibody 15
caggtacagctgcagcagtcaggggctgaggtgaagaagcctggctcttcggtgaaggtctcctgcaaggcttccggaggcaccttcagcagctacgctatcagctgggtgcgacaggcccctggacaagggcttgagtggatgggagggatcatccctacttttggtacagcaaactacgcacagaagttccagggcagagtcacgattaccgcggacgaatccacgagcacggcctacatggagctgagcggcctgaggtctgaggacacggccgtctattactgtgcgagagagaagaggcagtggctggaaggacactttgactactggggccggggcaccccggtcaccgtctcgagt

SEQ ID NO: 62 - protein, 121 aa, Homo sapiens; identical to SEQ ID NO: 14
  misc_feature (1)..(121): Antibody 15

SEQ ID NO: 63 - DNA, 321 bp, Homo sapiens
  misc_feature (1)..(321): Antibody 15
gacatccagatggctcaatccccatccttcctgtctgcatctattggagacagagtcaccatcacctgccgggccagtgagggtatttatcacaacttggcctggtatcagcagaagccggggaaagcccctaaactcctgatctatagggcctctagtttagccagtggggccccatcaaggttcagcggcagtggatctgggacggacttcactctcaccatcagcagcctgcagcctgatgattttgcaacttattactgccaacaatatagtaactatccgctcactttcggcggagggaccaagctggagatcaga

SEQ ID NO: 64 - protein, 107 aa, Homo sapiens
  misc_feature (1)..(107): Antibody 15
DIQMAQSPSFLSASIGDRVTITCRASEGIYHNLAWYQQKPGKAPKLLIYRASSLASGAPSRFSGSGSGTDFTLTISSLQPDDFATYYCQQYSNYPLTFGGGTKLEIR

SEQ ID NO: 65 - DNA, 363 bp, Homo sapiens
  misc_feature (1)..(363): Antibody 16
caggtacagctggtgcagtcaggggctgaggtgaagaagcctggctcttcggtgaaggtctcctgcaaggcttctggaggcaccttcagcagctacgctatcagctgggtgcgacaggcccctggacaagggcttgagtggatgggagggatcatccccacttttggtacagcaaactacgcacagaagtttcagggcagagtcacgattaccgcggacgaatccacgagcacagcctacatggagctgagcagcctgaggtctgaggacacggccgtctattactgcgcgagagagaagaggcagtggctggaaggacactttgactactggggccggggcaccctggtcaccgtctcctca

SEQ ID NO: 66 - protein, 121 aa, Homo sapiens; identical to SEQ ID NO: 10
  misc_feature (1)..(121): Antibody 16

SEQ ID NO: 67 - DNA, 321 bp, Homo sapiens
  misc_feature (1)..(321): Antibody 16
gacatccagatgacccagtctccatctaccctgtctgcatctgttggagacagagtcaccatcacctgccgggccagtgagagtatttatcactcgttggcctggtatcagcagaagccagggaaggcccctaaactcctgatctataaggcctctagtttagccagtggggtcccatcaaggttcagcggcagtagatctgggacagagttcactctcaccatcagcagcctgcagcctgatgattttgcgacttattactgccaacaatatagtaattatccgctcactttcggcggagggaccaaggtggagatcaaa

SEQ ID NO: 68 - protein, 107 aa, Homo sapiens
  misc_feature (1)..(107): Antibody 16
DIQMTQSPSTLSASVGDRVTITCRASESIYHSLAWYQQKPGKAPKLLIYKASSLASGVPSRFSGSRSGTEFTLTISSLQPDDFATYYCQQYSNYPLTFGGGTKVEIK

SEQ ID NO: 69 - DNA, 363 bp, Homo sapiens
  misc_feature (1)..(363): Antibody 17
caggtacagctgcagcagtcaggggctgaggtgaagaagcctggctcttcggtgaaagtctcctgcaaggcttctggaggcacctttagcagctacgctatcagctgggtgcgacaggcccctggacaagggcttgagtggatgggagggatcatccctactttcggtacagcaaactacgcacagaagttccagggcagagtcacgattaccgcggacgaatccacgagcacagcctacatggagctgagcggcctgaggtctgaggacacggccgtctattactgtgcgagagagaagaggcagtggctggaaggacactttgactactggggccggggcaccccggtcaccgtctcgagt

SEQ ID NO: 70 - protein, 121 aa, Homo sapiens; identical to SEQ ID NO: 14
  misc_feature (1)..(121): Antibody 17

SEQ ID NO: 71 - DNA, 321 bp, Homo sapiens
  misc_feature (1)..(321): Antibody 17
gacatccagatgacccagtccccatccttcctgtctgcatctatcggagacagagtcaccatcacctgccgggccagtgagggtatttatcacgagttggcctggtatcagcagaagccagggaaagcccctaaactcctgatctatagggcctctagtttagccagtggggccccatcaaggttcagcggcagtggatctgggacagatttcactctcaccatcagcagcctgcagcctgatgattttgcaacttattactgccaacaatatagtaattatccgctcactttcggcggagggaccaagctggagatcaaa

SEQ ID NO: 72 - protein, 107 aa, Homo sapiens
  misc_feature (1)..(107): Antibody 17
DIQMTQSPSFLSASIGDRVTITCRASEGIYHELAWYQQKPGKAPKLLIYRASSLASGAPSRFSGSGSGTDFTLTISSLQPDDFATYYCQQYSNYPLTFGGGTKLEIK

SEQ ID NO: 73 - DNA, 363 bp, Homo sapiens
  misc_feature (1)..(363): Antibody 18
caggtacagctgcagcagtcaggggctgaggtgaagaagcctggctcttcggtgaaggtctcctgcaaggcttctggaggcaccttcagcagctacgctatcagctgggtgcgacaggcccctggacaagggcttgagtggatgggagggatcatccctacttttggtacagcaaactacgcacagaagctccagggcagagtcacgattaccgcggacgaatccacgagcacagcctacatggagctgagcggcctgaggtctgaggacacggccgtctattactgtgcgagagagaagaggcagtggctggaaggacactttgactactggggccggggcaccccggtcaccgtctcgagt

SEQ ID NO: 74 - protein, 121 aa, Homo sapiens
  misc_feature (1)..(121): Antibody 18
QVQLQQSGAEVKKPGSSVKVSCKASGGTFSSYAISWVRQAPGQGLEWMGGIIPTFGTANYAQKLQGRVTITADESTSTAYMELSGLRSEDTAVYYCAREKRQWLEGHFDYWGRGTPVTVSS

SEQ ID NO: 75 - DNA, 321 bp, Homo sapiens
  misc_feature (1)..(321): Antibody 18
gacatccagatgacccagtccccatccttcctgtctgcatctattggagacagagtcaccatcacctgccgggccagtgagggtatttatcacgcgttggcctggtatcagcagaagccagggaaagccccaaaactcctgatctatagggcctctagtttagccagtggggccccatcaaggttcagcggcagtggatctgggacagatttcactctcaccatcagcagcctgcagcctgatgatttcgcgacttattactgccaacaatatagtaattatccgctcactttcggcggagggaccaagctggagatcaaa

SEQ ID NO: 76 - protein, 107 aa, Homo sapiens
  misc_feature (1)..(107): Antibody 18
DIQMTQSPSFLSASIGDRVTITCRASEGIYHALAWYQQKPGKAPKLLIYRASSLASGAPSRFSGSGSGTDFTLTISSLQPDDFATYYCQQYSNYPLTFGGGTKLEIK

SEQ ID NO: 77 - DNA, 363 bp, Homo sapiens
  misc_feature (1)..(363): Antibody 19
caggtacagctgcagcagtcaggggctgaggtgaagaagcctggctcttcggtgaaggtctcctgcaaggcttccggaggcaccttcagcagctacgctatcagctgggtgcgacaggcccctggacaagggcttgagtggatgggagggatcatccctatttttgatacagcaaactacgcacagaagttccagggcagagtcacgattaccgcggacgaatccacgagcacagcctacatggagctgagcggcctgaggtctgaggacacggccgtctattactgtgcgagagagaggaggcagtggctggaaggacactttgactactggggccggggcaccccggtcaccgtctcgagt

SEQ ID NO: 78 - protein, 121 aa, Homo sapiens
  misc_feature (1)..(121): Antibody 19
QVQLQQSGAEVKKPGSSVKVSCKASGGTFSSYAISWVRQAPGQGLEWMGGIIPIFDTANYAQKFQGRVTITADESTSTAYMELSGLRSEDTAVYYCARERRQWLEGHFDYWGRGTPVTVSS

SEQ ID NO: 79 - DNA, 321 bp, Homo sapiens
  misc_feature (1)..(321): Antibody 19
gacatccggatgacccagtccccgtccttcctgtctgcgtctattggagacagagtcaccatcacctgccgggccagtgagggtatttatcacgagttggcctggtatcagcagaagccagggaaagcccctaaactcctgatctatagggcctctagtttagccagtggggccccatcaaggttcagcggcagtggatctgggacagagttcactctcaccatcagcagcctgcagcctgatgattttgcaacttattactgccaacaatatagtaactatccgctcactttcggcggagggaccaagctggagatcaaa

SEQ ID NO: 80 - protein, 107 aa, Homo sapiens
  misc_feature (1)..(107): Antibody 19
DIRMTQSPSFLSASIGDRVTITCRASEGIYHELAWYQQKPGKAPKLLIYRASSLASGAPSRFSGSGSGTEFTLTISSLQPDDFATYYCQQYSNYPLTFGGGTKLEIK

SEQ ID NO: 81 - DNA, 363 bp, Homo sapiens
  misc_feature (1)..(363): Antibody 20
caggtacagctgcagcagtcaggggctgaggtgaagaagcctggctcttcagtgaaggtctcctgcaaggcttctggaggcaccttcagcagctacgctatcagctgggtgcgacaggcccctggacaagggcttgagtggatgggagggatcatccctacttttggtacagcaaactacgcacagaagttccagggcagagtcacgattaccgcggacgaatccacgagcacagcctacatggagctgagcagcctgaggtctgaggacacggccgtctattactgtgcgagagagaagaggcagtggctggaaggacactttgactactggggccggggcaccctggtcaccgtctcctca

SEQ ID NO: 82 - protein, 121 aa, Homo sapiens
  misc_feature (1)..(121): Antibody 20
QVQLQQSGAEVKKPGSSVKVSCKASGGTFSSYAISWVRQAPGQGLEWMGGIIPTFGTANYAQKFQGRVTITADESTSTAYMELSSLRSEDTAVYYCAREKRQWLEGHFDYWGRGTLVTVSS

SEQ ID NO: 83 - DNA, 321 bp, Homo sapiens
  misc_feature (1)..(321): Antibody 20
gacatccagatgacccagtccccatccaccctgtctgcatctgttggagacagagtcaccatcacctgccgggccagtgagggtatttatcacgagttggcctggtatcagcagaagccagggaaagccccaaagctcctgatctatagggcctctagtttagccagtggggtcccatcaaggttcagcggcagtagatctgggacagagttcactctcaccatcagcagcctgcagcctgatgattttgcgacttattactgccaacaatatagcgattatccgctcactttcggcggggggaccaaggtggagatcaaa

SEQ ID NO: 84 - protein, 107 aa, Homo sapiens
  misc_feature (1)..(107): Antibody 20
DIQMTQSPSTLSASVGDRVTITCRASEGIYHELAWYQQKPGKAPKLLIYRASSLASGVPSRFSGSRSGTEFTLTISSLQPDDFATYYCQQYSDYPLTFGGGTKVEIK

SEQ ID NO: 85 - DNA, 363 bp, Homo sapiens
  misc_feature (1)..(363): Antibody 21
ccagtacagctgcagcagtcaggggctgaggtgaagaagcctggctcttcggtgaaggtctcctgcaaggcttctggaggtaccttcagcagctacgctatcagctgggtgcgacaggcccctggacaagggcttgagtggatgggagggatcatccctacttttggtacagcaaactacgcacagaagttccagggcagagtcacgattaccgcggacgaatccacgagcacggcctacatggagctgagcggcctgaggtctgaggacacggccgtctactactgtgcgagagagaagaggcagtggctggaaggacactttgactactggggccggggcaccccggtcaccgtctcgagt

SEQ ID NO: 86 - protein, 121 aa, Homo sapiens
  misc_feature (1)..(121): Antibody 21
PVQLQQSGAEVKKPGSSVKVSCKASGGTFSSYAISWVRQAPGQGLEWMGGIIPTFGTANYAQKFQGRVTITADESTSTAYMELSGLRSEDTAVYYCAREKRQWLEGHFDYWGRGTPVTVSS

SEQ ID NO: 87 - DNA, 321 bp, Homo sapiens
  misc_feature (1)..(321): Antibody 21
gacatccagatggcccagtccccatccttcctgtctgcatctattggagacagagtcaccatcacctgccgggccagtgagggtatttatcacaacttggcctggtatcagcagaagccagggaaagccccaaaactcctgatctatagggcctctagtttagccagtggggccccatcaaggttcagcggcagcggatctgggacagatttcactctcaccatcagcagcctgcagcctgatgattttgcaacttattactgccaacaatatagtaattatccgctcactttcggcggagggaccaagctggagatcaaa

SEQ ID NO: 88 - protein, 107 aa, Homo sapiens
  misc_feature (1)..(107): Antibody 21
DIQMAQSPSFLSASIGDRVTITCRASEGIYHNLAWYQQKPGKAPKLLIYRASSLASGAPSRFSGSGSGTDFTLTISSLQPDDFATYYCQQYSNYPLTFGGGTKLEIK

SEQ ID NO: 89 - DNA, 363 bp, Homo sapiens
  misc_feature (1)..(363): Antibody 22
caggtacagctgcagcagtcaggggctgaggtgaagaagcctggctcttcggtgaaggtctcctgcaaggcttctggaggcaccttcagcggctacgctatcagctgggtgcgacaggcccctggacaggggctcgagtggatgggagggatcatccctacttttggtacagcaaactactcacagaagttccagggcagagtcacgattaccgcggacgaatccacgagcacagcctacatggagctgagcggcctgaggtctgaggacacggccgtctattactgtgcgagagagaagaggcagtggctggaaggacactttgactactggggccggggcaccccggtcaccgtctcgagt

SEQ ID NO: 90 - protein, 121 aa, Homo sapiens
  misc_feature (1)..(121): Antibody 22
QVQLQQSGAEVKKPGSSVKVSCKASGGTFSGYAISWVRQAPGQGLEWMGGIIPTFGTANYSQKFQGRVTITADESTSTAYMELSGLRSEDTAVYYCAREKRQWLEGHFDYWGRGTPVTVSS

SEQ ID NO: 91 - DNA, 321 bp, Homo sapiens
  misc_feature (1)..(321): Antibody 22
gacatccagatgacccagtccctatccttcctgtctgcatctattggggacagagtcaccatcacctgccgggccagtgagggtatttatcacgccttggcctggtatcagcagaagccagggaaagccccaaaactcctgatctatagggcctctagtttagccagtggggccccatcaaggttcagcggcagtggatctgggacagatttcactctcaccatcagcagcctgcagcctgatgattttgcgacttattactgccaacaatatagtaattatccgctcactttcggcggagggaccaagctggagatcaaa

SEQ ID NO: 92 - protein, 107 aa, Homo sapiens
  misc_feature (1)..(107): Antibody 22
DIQMTQSLSFLSASIGDRVTITCRASEGIYHALAWYQQKPGKAPKLLIYRASSLASGAPSRFSGSGSGTDFTLTISSLQPDDFATYYCQQYSNYPLTFGGGTKLEIK

SEQ ID NO: 93 - DNA, 363 bp, Homo sapiens
  misc_feature (1)..(363): Antibody 23
caggtacagctgcagcagtcaggggctgaggtgaagaagcctggctcttcggtgaaggtctcctgcaaggcttctggaggcaccttcagcagctacgctatcagctgggtgcgacaggcccctggacaagggcttgagtggatgggagggatcatccctactttcggtacagcaaactacgcacagaagttccagagcagagtcacgattaccgcggacgaatccacgagcacagcctacatggagctgagcggcctgaggtccgaggacacggccgtctattactgtgcgagagagaagaggcagtggctggaaggacactttgactactggggccggggcaccccggtcaccgtctcgagt

SEQ ID NO: 94 - protein, 121 aa, Homo sapiens
  misc_feature (1)..(121): Antibody 23
QVQLQQSGAEVKKPGSSVKVSCKASGGTFSSYAISWVRQAPGQGLEWMGGIIPTFGTANYAQKFQSRVTITADESTSTAYMELSGLRSEDTAVYYCAREKRQWLEGHFDYWGRGTPVTVSS

SEQ ID NO: 95 - DNA, 321 bp, Homo sapiens
  misc_feature (1)..(321): Antibody 23
gacatccagatgacccagtccccatccttcctgtctgcatctattggagacagagtcaccatcacctgccgggccagcgagggtatttatcacagcttggcctggtatcagcagaagccagggaaagccccaaaactcctgatctatagggcctctagtttagccagtggggccccatcaaggttcagcggcagtggatctgggacagatttcactctcaccatcagcagcctgcagcctgatgattttgcgacttattactgccaacaatatagtaattatccgctcactttcggcggagggaccaagctggagatcaaa

SEQ ID NO: 96 - protein, 107 aa, Homo sapiens; identical to SEQ ID NO: 56
  misc_feature (1)..(107): Antibody 23

SEQ ID NO: 97 - DNA, 363 bp, Homo sapiens
  misc_feature (1)..(363): Antibody 24
caggtacagctgcagcagtcaggggctgaggtgaagaagcctggctcttcggtgaaggtctcctgcaaggcttctggaggtaccttcagcagctacgctatcagctgggtgcgacaggcccctgggcaagggcttgagtggatgggagggatcatccctacttttgtgacagcaaactacgcacagaagttccagggcagagtctcgattaccgcggacgaatccacgagcacagcctacatggagctgagcggcctgaggtctgaggacacggccgtctattactgtgcgagagagaagaggcagtggctggaaggacactttgactactggggccggggcaccccggtcaccgtctcgagt

SEQ ID NO: 98 - protein, 121 aa, Homo sapiens
  misc_feature (1)..(121): Antibody 24
QVQLQQSGAEVKKPGSSVKVSCKASGGTFSSYAISWVRQAPGQGLEWMGGIIPTFVTANYAQKFQGRVSITADESTSTAYMELSGLRSEDTAVYYCAREKRQWLEGHFDYWGRGTPVTVSS

SEQ ID NO: 99 - DNA, 321 bp, Homo sapiens
  misc_feature (1)..(321): Antibody 24
gacatccagatgacccagtccccatccttcctgtctgcatctattggagacagagtcaccatcacctgccgggccagtgagggtatttatcacgcgttggcctggtatcagcagaagccggggaaagccccaaaactcctgatctatagggcctctagtttagccagtggggccccatcaaggttcagcggcagtggatctgggacagatttcactctcaccatcagcagcctgcagcctgatgattttgcgacttattactgccaacaatatagtaattatccgctcactttcggcggagggaccaagctggagatcaaa

SEQ ID NO: 100 - protein, 107 aa, Homo sapiens; identical to SEQ ID NO: 76
  misc_feature (1)..(107): Antibody 24

SEQ ID NO: 101 - DNA, 363 bp, Homo sapiens
  misc_feature (1)..(363): Antibody 25
caggtacagctgcagcagtcaggggctgaggtgaagaagcctggctcttcggtgaaggtctcctgcaaggcttctggaggcaccttcagcagctacgctatcagctgggtgcgacaggcccctggacaagggcttgagtggatgggagggatcatccctacttttggtacagcaaactacgcacagaagttccagggcagagtcacgattaccgcggacgaatccacgagcacagcctacatggagctgagcggcctgaggtctgaggacacggccgtctattactgtgcgagagagaggaggcagtggctggaaggacactttgactactggggccagggcaccccggtcaccgtctcgagt

SEQ ID NO: 102 - protein, 121 aa, Homo sapiens
  misc_feature (1)..(121): Antibody 25
QVQLQQSGAEVKKPGSSVKVSCKASGGTFSSYAISWVRQAPGQGLEWMGGIIPTFGTANYAQKFQGRVTITADESTSTAYMELSGLRSEDTAVYYCARERRQWLEGHFDYWGQGTPVTVSS

SEQ ID NO: 103 - DNA, 321 bp, Homo sapiens
  misc_feature (1)..(321): Antibody 25
gacatccagatgacccagtccccatccttcctgtctgcatctattggagacagagtcaccatcacctgccgggccagtgagagtatttatcacgagttggcctggtatcagcagaagccagggaaagccccaaaactcctgatctataaggcctctagtttagccagcggggccccatcaaggttcagcggcagtgggtctgggacagctttcactcttaccatcagcagcctgcagcctgatgattttgcgacttattactgccaacaatatagtaattatccgctcactttcggcggagggaccaagctggagatcaaa

SEQ ID NO: 104 - protein, 107 aa, Homo sapiens
  misc_feature (1)..(107): Antibody 25
DIQMTQSPSFLSASIGDRVTITCRASESIYHELAWYQQKPGKAPKLLIYKASSLASGAPSRFSGSGSGTAFTLTISSLQPDDFATYYCQQYSNYPLTFGGGTKLEIK

SEQ ID NO: 105 - DNA, 363 bp, Homo sapiens
  misc_feature (1)..(363): Antibody 26
caggtacagctgcagcagtccggggctgaggtgaagaagcctggcacttcggtgaaggtctcctgcaaggcttccggaggcaccttcagcagctatgctatcagctgggtgcgacaggcccctggacaagggcttgagtggatgggagggatcatccctattttcggtacagcaaactacgcacagaagttccagggcagagtcacgattaccgcggacgaatccacgagcacagcctacatggagctgagcagcctgagatctgaggacacggccgtctattactgtgcgagagagaagaggcagtggctggaaggacactttgactactggggccggggcaccccggtcaccgtctcgagt

SEQ ID NO: 106 - protein, 121 aa, Homo sapiens
  misc_feature (1)..(121): Antibody 26
QVQLQQSGAEVKKPGTSVKVSCKASGGTFSSYAISWVRQAPGQGLEWMGGIIPIFGTANYAQKFQGRVTITADESTSTAYMELSSLRSEDTAVYYCAREKRQWLEGHFDYWGRGTPVTVSS

SEQ ID NO: 107 - DNA, 321 bp, Homo sapiens
  misc_feature (1)..(321): Antibody 26
gacatccagatgacccagtccccatccttcctgtctgcatctattggagacagagtcaccatcacctgccgggccagtgagggtatttatcacacgttggcctggtatcagcagaagccagggaaagccccaaagctcctgatctatagggcctctagtttagccagtggggccccatcaaggttcagcggcagtggatctgggacagatttcactctcaccatcagtagcctgcagcctgatgattttgcgacttattactgccaacaatatagtaattatccgctcactttcggcggagggaccaagctggagatcaaa

SEQ ID NO: 108 - protein, 107 aa, Homo sapiens
  misc_feature (1)..(107): Antibody 26
DIQMTQSPSFLSASIGDRVTITCRASEGIYHTLAWYQQKPGKAPKLLIYRASSLASGAPSRFSGSGSGTDFTLTISSLQPDDFATYYCQQYSNYPLTFGGGTKLEIK

SEQ ID NO: 109 - DNA, 363 bp, Homo sapiens
  misc_feature (1)..(363): Antibody 27
caggtacagctgcagcagtcaggggctgaggtgaagaagcctggctcttcggtgaaggtctcctgcaaggcttctggaggcgccttcagcagctacgctatcagctgggtgcgacaggcccctggacaagggcttgagtggatgggagggatcatccctacctttggtacagcaaactacgcacagaagttccagggcagagtcacgattaccgcggacgaatccacgagcacagcctacatggagctgagcggcctgaggtctgaggacacggccgtctattactgtgcgagagagaagaggcagtggctggaaggacactttgactactggggccggggcaccccggtcaccgtctcctca

SEQ ID NO: 110 - protein, 121 aa, Homo sapiens
  misc_feature (1)..(121): Antibody 27
QVQLQQSGAEVKKPGSSVKVSCKASGGAFSSYAISWVRQAPGQGLEWMGGIIPTFGTANYAQKFQGRVTITADESTSTAYMELSGLRSEDTAVYYCAREKRQWLEGHFDYWGRGTPVTVSS

SEQ ID NO: 111 - DNA, 321 bp, Homo sapiens
  misc_feature (1)..(321): Antibody 27
gacatccagatgacccagtccccatccttcctgtctgcatctattggagacagagtcaccatcacctgccgggccagtgagggtatttatcacaacttggcctggtatcagcagaagccagggaaagcccctaaactcctgatctatagggcctctagtttagccagtggggccccatcaaggttcagcggcagtggatctgggacagatttcactctcaccatcagcagcctgcagcctgatgattttgcaacttattactgccaacaatatagtaattatccgctcactttcggcggagggaccaagctggagatcaaa

SEQ ID NO: 112 - protein, 107 aa, Homo sapiens; identical to SEQ ID NO: 52
  misc_feature (1)..(107): Antibody 27

SEQ ID NO: 113 - protein, 5 aa, Homo sapiens
  misc_feature (1)..(5): Antibody 1-27
SYAIS

SEQ ID NO: 114 - protein, 17 aa, Homo sapiens
  misc_feature (1)..(17): Antibody 19
GIIPIFDTANYAQKFQG

SEQ ID NO: 115 - protein, 12 aa, Homo sapiens
  misc_feature (1)..(12): Antibodies 1, 2, 3, 5-7, 10, 12-18, 20-24, 26, and 27
EKRQWLEGHFDY

SEQ ID NO: 116 - skipped

SEQ ID NO: 117 - protein, 7 aa, Homo sapiens
  misc_feature (1)..(7): Antibodies 1, 2, 3, 8, 16 and 25
KASSLAS

SEQ ID NO: 118 - protein, 9 aa, Homo sapiens
  misc_feature (1)..(9): Antibodies 1, 3-19, and 21-27
QQYSNYPLT

SEQ ID NO: 119 - protein, 402 aa, Homo sapiens [sequence omitted]

SEQ ID NO: 120 - protein, 402 aa, Rattus norvegicus
  misc_feature (1)..(402): PAI-1
MQMSSALTCLTLGLVLVFGKGFASPLPESHTAQQATNFGVKVFQHVVQASKDRNVVFSPYGVSSVLAMLQLTTAGKTRQQIQDAMGFNISERGTAPALRKLSKELMGSWNKNEISTADAIFVQRDLELVQGFMPHFFKLFRTTVKQVDFSEVERARFIINDWVERHTKGMISDLLAKGAVNELTRLVLVNALYFNGQWKTPFLEASTHQRLFHKSDGSTISVPMMAQNNKFNYTEFTTPDGHEYDILELPYHGETLSMFIAAPFEKDVPLSAITNILDAELIRQWKSNMTRLPRLLILPKFSLETEVDLRGPLEKLGMTDIFSSTQADFTSLSDQEQLSVAQALQKVKIEVNESGTVASSSTAILVSARMAPTEMVLDRSFLFVVRHNPTETILFMGQLMEP

SEQ ID NO: 121 - protein, 402 aa, Mus musculus
  misc_feature (1)..(402): PAI-1
MQMSSALACLILGLVLVSGKGFALPLRESHTAHQATDFGVKVFQQVVQASKDRNVVFSPYGVSSVLAMLQMTTAGKTRRQIQDAMGFKVNEKGTAHALRQLSKELMGPWNKNEISTADAIFVQRDLELVQGFMPHFFKLFQTMVKQVDFSEVERARFIINDWVERHTKGMINDLLAKGAVDELTRLVLVNALYFSGQWKTPFLEASTHQRLFHKSDGSTVSVPMMAQSNKFNYTEFTTPDGLEYDVVELPYQGDTLSMFIAAPFEKDVHLSALTNILDAELIRQWKGNMTRLPRLLILPKFSLETEVDLRGPLEKLGMPDMFSATLADFTSLSDQEQLSVAQALQKVRIEVNESGTVASSSTAFVISARMAPTEMVIDRSFLFVVRHNPTETILFMGQVMES

SEQ ID NO: 122 - protein, 402 aa, Macaca fascicularis
  misc_feature (1)..(402): PAI-1
MQMSPALACLVLGLAFVFGEGSTVHHPPSYVAHLASDFGVRVFQQVAQASKDRNVVFSPYGVASVLAMLQLTTGGETRQQIQAAMGFKIDDKGMAPALRHLYKELLGPWNKDEISTTDAIFVQRDLKLVQGFMPHFFRLFRSTVKQVDFSEAERARFIINDWVKTHTKGMISDLLGKGAVDQLTRLVLVNALYFNGHWKTPFPDSSTHRRLFHKSDGSTVSVPMMAQTNKFNYTEFTTPDGHYYDILELPYHGNTLSMFIAAPYEKQVPLSALTNILSAQLISHWKGNMTRLPRLLVLPKFSLETEVDLRKPLENLGMTDMFRQFQADFTSLSNQEPLHVAQALQKVKIEVNESGTVASSSTAVIVSARMAPEEIIMDRPFLFVVRHNPTGTVLFMGQVMEP

SEQ ID NO: 123 - protein, 400 aa, Canis familiaris
  misc_feature (1)..(400): PAI-1
MGSSHHHHHHSSGLVPRGSHMSYSYQTRAAGLATDFGVKVFQEVARASKDRNMVFSPYGVASVLAMLQLTTAGETCRQIQEAMQFQIDEKGMAPALRQLYKELMGPWNKDEISTADAIFVQRDLKLVHGFMPHFFRLFRTTVKQVDFSEVERARFIVNDWVKRHTKGMIGNLLGRGAVDQLTRLMLVNALYFNGQWKTPFPESGTHHRLFHKSDGSTVSVPMMAQTNKFNYTEFSTPSGHYYDILELPYHGDTLSMFIAAPYEKEVPLSALTNILDAQLISQWKGNMTRQLRLLVLPKFSLETEVNLRRPLENLGMTDMFRPNLADFSSLSNQEVLYVSRALQKVKIEVNESGTVASSSTAIIVSARMAPEEIIMDRPFLFVVRHNPTGTVLFMGQVMEP

SEQ ID NO: 124 - protein, 379 aa, Homo sapiens [sequence omitted]

SEQ ID NO: 125 - protein, 8 aa, Artificial sequence
  Synthetic sequence: Derived from human PAI-1 sequence SEQ ID NO: 124
CGHYYDIC

SEQ ID NO: 126 - protein, 12 aa, Artificial sequence
  Synthetic sequence: Derived from human PAI-1 sequence SEQ ID NO: 124
CGHYYDCGHYDC

SEQ ID NO: 127 - protein, 15 aa, Artificial sequence
  Synthetic sequence: Derived from human PAI-1 sequence SEQ ID NO: 124
CGHYYDGCGGHYDGC

SEQ ID NO: 128 - protein, 13 aa, Artificial sequence
  Synthetic sequence: Derived from human PAI-1 sequence SEQ ID NO: 124
CGHYYDCNYTEFC

SEQ ID NO: 129 - protein, 9 aa, Artificial sequence
  Synthetic sequence: Derived from human PAI-1 sequence SEQ ID NO: 124
CGHYYDILC

SEQ ID NO: 130 - protein, 9 aa, Artificial sequence
  Synthetic sequence: Derived from human PAI-1 sequence SEQ ID NO: 124
CDGHYYDIC

SEQ ID NO: 131 - protein, 13 aa, Artificial sequence
  Synthetic sequence: Derived from human PAI-1 sequence SEQ ID NO: 124
CNYTEFCGHYYDC

SEQ ID NO: 132 - protein, 21 aa, Artificial sequence
  Synthetic sequence: Derived from human PAI-1 sequence SEQ ID NO: 124
CRNVVFSPYGCASVLAMLQLC

SEQ ID NO: 133 - protein, 21 aa, Artificial sequence
  Synthetic sequence: Derived from human PAI-1 sequence SEQ ID NO: 124
CVVFSPYGVACVLAMLQLTTC

SEQ ID NO: 134 - protein, 13 aa, Artificial sequence
  Synthetic sequence: Derived from human PAI-1 sequence SEQ ID NO: 124
CGHYYDCGHYYDC

SEQ ID NO: 135 - protein, 16 aa, Artificial sequence
  Synthetic sequence: Derived from human PAI-1 sequence SEQ ID NO: 124
CGHYYDGCGGHYYDGC

SEQ ID NO: 136 - protein, 20 aa, Artificial sequence
  Synthetic sequence: Derived from human PAI-1 sequence SEQ ID NO: 124
CRNVVFSPYGCASVLAMQLC

SEQ ID NO: 137 - protein, 10 aa, Artificial sequence
  Synthetic sequence: Derived from human PAI-1 sequence SEQ ID NO: 124
CHYYDILELC

SEQ ID NO: 138 - protein, 9 aa, Artificial sequence
  Synthetic sequence: Derived from human PAI-1 sequence SEQ ID NO: 124
CYYDILELC

SEQ ID NO: 139 - protein, 8 aa, Artificial sequence
  Synthetic sequence: Derived from human PAI-1 sequence SEQ ID NO: 124
CYYDILEC

SEQ ID NO: 140 - protein, 20 aa, Artificial sequence
  Synthetic sequence: Derived from human PAI-1 sequence SEQ ID NO: 124
CFNYTEFTTCGHYYDILELC

SEQ ID NO: 141 - protein, 8 aa, Artificial sequence
  Synthetic sequence: Derived from human PAI-1 sequence SEQ ID NO: 124
CHYYDILC

SEQ ID NO: 142 - protein, 21 aa, Artificial sequence
  Synthetic sequence: Derived from human PAI-1 sequence SEQ ID NO: 124
CGAVDQLTRLCLVNALYFNGC

SEQ ID NO: 143 - protein, 17 aa, Artificial sequence
  Synthetic sequence: Derived from human PAI-1 sequence SEQ ID NO: 124
CQTNKFNYCTRLPRLLC

SEQ ID NO: 144 - protein, 9 aa, Artificial sequence
  Synthetic sequence: Derived from human PAI-1 sequence SEQ ID NO: 124
CHYYDILEC

SEQ ID NO: 145 - protein, 15 aa, Artificial sequence
  Synthetic sequence: Derived from human PAI-1 sequence SEQ ID NO: 124
CEFTTCGHYYDILEC

SEQ ID NO: 146 - protein, 21 aa, Artificial sequence
  Synthetic sequence: Derived from human PAI-1 sequence SEQ ID NO: 124
CLFVVRHNPTCTVLFMGQVMC

SEQ ID NO: 147 - protein, 10 aa, Artificial sequence
  Synthetic sequence: Derived from human PAI-1 sequence SEQ ID NO: 124
CGHYYDILEC

SEQ ID NO: 148 - protein, 16 aa, Artificial sequence
  Synthetic sequence: Derived from human PAI-1 sequence SEQ ID NO: 124
CQTNKFNYCTRLPRLC

SEQ ID NO: 149 - protein, 21 aa, Artificial sequence
  Synthetic sequence: Derived from human PAI-1 sequence SEQ ID NO: 124
CNVVFSPYGVCSVLAMLQLTC

SEQ ID NO: 150 - protein, 11 aa, Artificial sequence
  Synthetic sequence: Derived from human PAI-1 sequence SEQ ID NO: 124
CGHYYDILELC

SEQ ID NO: 151 - protein, 15 aa, Artificial sequence
  Synthetic sequence: Derived from human PAI-1 sequence SEQ ID NO: 124
CTNKFNYCTRLPRLC

SEQ ID NO: 152 - protein, 14 aa, Artificial sequence
  Synthetic sequence: Derived from human PAI-1 sequence SEQ ID NO: 124
CFTTPDGHYYDILC

SEQ ID NO: 153 - protein, 18 aa, Artificial sequence
  Synthetic sequence: Derived from human PAI-1 sequence SEQ ID NO: 124
CQTNKFNYTECNMTRLPC

SEQ ID NO: 154 - protein, 21 aa, Artificial sequence
  Synthetic sequence: Derived from human PAI-1 sequence SEQ ID NO: 124
CFNYTEFTTPCGHYYDILELC

SEQ ID NO: 155 - protein, 17 aa, Artificial sequence
  Synthetic sequence: Derived from human PAI-1 sequence SEQ ID NO: 124
CTNKFNYCTRLPRLLVC

SEQ ID NO: 156 - protein, 5 aa, Homo sapiens
GHYYD

SEQ ID NO: 157 - protein, 7 aa, Homo sapiens
QTNKFNY

SEQ ID NO: 158 - protein, 7 aa, Homo sapiens
TRLPRLL

SEQ ID NO: 159 - protein, 5 aa, Homo sapiens
  misc_feature (1)..(5): HCDR2 of Antibody 22
GYAIS

SEQ ID NO: 160 - protein, 17 aa, Homo sapiens
  misc_feature (1)..(17): HCDR2 of Antibodies 01, 11, 26
GIIPIFGTANYAQKFQG

SEQ ID NO: 161 - protein, 17 aa, Homo sapiens
  misc_feature (1)..(17): HCDR2 of Antibodies 02, 03, 06..08, 10, 13..17, 20, 21, 25, 27
GIIPTFGTANYAQKFQG

SEQ ID NO: 162 - protein, 17 aa, Homo sapiens
  misc_feature (1)..(17): HCDR2 of Antibody 04
GIIPTFGTANYAQKFRG

SEQ ID NO: 163 - protein, 17 aa, Homo sapiens
  misc_feature (1)..(17): HCDR2 of Antibody 05
GIIPTFSTANYAQKFRG

SEQ ID NO: 164 - protein, 17 aa, Homo sapiens
  misc_feature (1)..(17): HCDR2 of Antibody 09
GIIPAFDTANYAQKFQG

SEQ ID NO: 165 - protein, 17 aa, Homo sapiens
  misc_feature (1)..(17): HCDR2 of Antibody 12
GIIPTFPTANYAQKFQG

SEQ ID NO: 166 - protein, 17 aa, Homo sapiens
  misc_feature (1)..(17): HCDR2 of Antibody 18
GIIPTFGTANYAQKLQG

SEQ ID NO: 167 - protein, 17 aa, Homo sapiens
  misc_feature (1)..(17): HCDR2 of Antibody 22
GIIPTFGTANYSQKFQG

SEQ ID NO: 168 - protein, 17 aa, Homo sapiens
  misc_feature (1)..(17): HCDR2 of Antibody 23
GIIPTFGTANYAQKFQS

SEQ ID NO: 169 - protein, 17 aa, Homo sapiens
  misc_feature (1)..(17): HCDR2 of Antibody 24
GIIPTFVTANYAQKFQG

SEQ ID NO: 170 - protein, 12 aa, Homo sapiens
  misc_feature (1)..(12): HCDR3 of Antibody 04
EKRQWLEGRFDY

SEQ ID NO: 171 - protein, 12 aa, Homo sapiens
  misc_feature (1)..(12): HCDR3 of Antibodies 08, 19, 25
ERRQWLEGHFDY

SEQ ID NO: 172 - protein, 11 aa, Homo sapiens
  misc_feature (1)..(11): LCDR1 of Antibodies 01, 03
RASEGIYRSLA

SEQ ID NO: 173 - protein, 11 aa, Homo sapiens
  misc_feature (1)..(11): LCDR1 of Antibodies 02, 04, 10, 13, 23
RASEGIYHSLA

SEQ ID NO: 174 - protein, 11 aa, Homo sapiens
  misc_feature (1)..(11): LCDR1 of Antibody 05
RASEGIYHQLA

SEQ ID NO: 175 - protein, 11 aa, Homo sapiens
  misc_feature (1)..(11): LCDR1 of Antibodies 06, 07, 17, 19, 20
RASEGIYHELA

SEQ ID NO: 176 - protein, 11 aa, Homo sapiens
  misc_feature (1)..(11): LCDR1 of Antibodies 08, 12, 14, 15, 21, 27
RASEGIYHNLA

SEQ ID NO: 177 - protein, 11 aa, Homo sapiens
  misc_feature (1)..(11): LCDR1 of Antibody 09
QASEGIYHNLA

SEQ ID NO: 178 - protein, 11 aa, Homo sapiens
  misc_feature (1)..(11): LCDR1 of Antibody 11
QASEGIYHELA

SEQ ID NO: 179 - protein, 11 aa, Homo sapiens
  misc_feature (1)..(11): LCDR1 of Antibody 16
RASESIYHSLA

SEQ ID NO: 180 - protein, 11 aa, Homo sapiens
  misc_feature (1)..(11): LCDR1 of Antibodies 18, 22, 24
RASEGIYHALA

SEQ ID NO: 181 - protein, 11 aa, Homo sapiens
  misc_feature (1)..(11): LCDR1 of Antibody 25
RASESIYHELA

SEQ ID NO: 182 - protein, 11 aa, Homo sapiens
  misc_feature (1)..(11): LCDR1 of Antibody 26
RASEGIYHTLA

SEQ ID NO: 183 - protein, 7 aa, Homo sapiens
  misc_feature (1)..(7): LCDR2 of Antibody 04
RATSLAS

SEQ ID NO: 184 - protein, 7 aa, Homo sapiens
  misc_feature (1)..(7): LCDR2 of Antibodies 05..07, 09..15, 17..24, 26, 27
RASSLAS

SEQ ID NO: 185 - protein, 9 aa, Homo sapiens
  misc_feature (1)..(9): LCDR3 of Antibody 02, 20
QQYSDYPLT

SEQ ID NO: 186 - protein, 9 aa, Homo sapiens
  misc_feature (1)..(9): LCDR3 of Antibody 08
QQYSNYPLT

SEQ ID NO: 187 - protein, 5 aa, Homo sapiens
  misc_feature (1)..(5): HCDR1 of Antibody 08
SYAIS

SEQ ID NO: 188 - protein, 7 aa, Artificial sequence
  Synthetic sequence: Changed motif in R271S mutant PAI-1
TRLPSLL

SEQ ID NO: 189 - protein, 379 aa, Artificial sequence
  Synthetic sequence: R271S mutant PAI-1
VHHPPSYVAHLASDFGVRVFQQVAQASKDRNVVFSPYGVASVLAMLQLTTGGETQQQIQAAMGFKIDDKGMAPALRHLYKELMGPWNKDEISTTDAIFVQRDLKLVQGFMPHFFRLFRSTVKQVDFSEVERARFIINDWVKTHTKGMISNLLGKGAVDQLTRLVLVNALYFNGQWKTPFPDSSTHRRLFHKSDGSTVSVPMMAQTNKFNYTEFTTPDGHYYDILELPYHGDTLSMFIAAPYEKEVPLSALTNILSAQLISHWKGNMTRLPSLLVLPKFSLETEVDLRKPLENLGMTDMFRQFQADFTSLSDQEPLHVAQALQKVKIEVNESGTVASSSTAVIVSARMAPEEIIMDRPFLFVVRHNPTGTVLFMGQVMEP

SEQ ID NO: 190 - protein, 400 aa, Homo sapiens
  misc_feature (1)..(400): Stable form of human PAI-1
MGSSHHHHHHSSGLVPRGSHMVHHPPSYVAHLASDFGVRVFQQVAQASKDRNVVFSPYGVASVLAMLQLTTGGETQQQIQAAMGFKIDDKGMAPALRHLYKELMGPWNKDEISTTDAIFVQRDLKLVQGFMPHFFRLFRSTVKQVDFSEVERARFIINDWVKTHTKGMISHLLGTGAVDQLTRLVLVNALYFNGQWKTPFPDSSTHRRLFHKSDGSTVSVPMMAQTNKFNYTEFTTPDGHYYDILELPYHGDTLSMFIAAPYEKEVPLSALTNILSAQLISHWKGNMTRLPRLLVLPKFSLETEVDLRKPLENLGMTDMFRQFQADFTSLSDQEPLHVALALQKVKIEVNESGTVASSSTAVIVSARMAPEEIIIDRPFLFVVRHNPTGTVLFMGQVMEP

SEQ ID NO: 191 - protein, 400 aa, Artificial sequence
  Synthetic sequence: R292S mutant of stable form of human PAI-1
MGSSHHHHHHSSGLVPRGSHMVHHPPSYVAHLASDFGVRVFQQVAQASKDRNVVFSPYGVASVLAMLQLTTGGETQQQIQAAMGFKIDDKGMAPALRHLYKELMGPWNKDEISTTDAIFVQRDLKLVQGFMPHFFRLFRSTVKQVDFSEVERARFIINDWVKTHTKGMISHLLGTGAVDQLTRLVLVNALYFNGQWKTPFPDSSTHRRLFHKSDGSTVSVPMMAQTNKFNYTEFTTPDGHYYDILELPYHGDTLSMFIAAPYEKEVPLSALTNILSAQLISHWKGNMTRLPSLLVLPKFSLETEVDLRKPLENLGMTDMFRQFQADFTSLSDQEPLHVALALQKVKIEVNESGTVASSSTAVIVSARMAPEEIIIDRPFLFVVRHNPTGTVLFMGQVMEP

SEQ ID NO: 192 - protein, 4 aa, Homo sapiens
LPRL

SEQ ID NO: 193 - protein, 6 aa, Homo sapiens
RMAPEE

SEQ ID NO: 194 - protein, 400 aa, Homo sapiens
  misc_feature (1)..(4): Y210N stable mutant human PAI-1
MGSSHHHHHHSSGLVPRGSHMVHHPPSYVAHLASDFGVRVFQQVAQASKDRNVVFSPYGVASVLAMLQLTTGGETQQQIQAAMGFKIDDKGMAPALRHLYKELMGPWNKDEISTTDAIFVQRDLKLVQGFMPHFFRLFRSTVKQVDFSEVERARFIINDWVKTHTKGMISHLLGTGAVDQLTRLVLVNALYFNGQWKTPFPDSSTHRRLFHKSDGSTVSVPMMAQTNKFNNTEFTTPDGHYYDILELPYHGDTLSMFIAAPYEKEVPLSALTNILSAQLISHWKGNMTRLPRLLVLPKFSLETEVDLRKPLENLGMTDMFRQFQADFTSLSDQEPLHVALALQKVKIEVNESGTVASSSTAVIVSARMAPEEIIIDRPFLFVVRHNPTGTVLFMGQVMEP

SEQ ID NO: 195 - protein, 400 aa, Homo sapiens
  misc_feature (1)..(400): E212V stable mutant human PAI-1
MGSSHHHHHHSSGLVPRGSHMVHHPPSYVAHLASDFGVRVFQQVAQASKDRNVVFSPYGVASVLAMLQLTTGGETQQQIQAAMGFKIDDKGMAPALRHLYKELMGPWNKDEISTTDAIFVQRDLKLVQGFMPHFFRLFRSTVKQVDFSEVERARFIINDWVKTHTKGMISHLLGTGAVDQLTRLVLVNALYFNGQWKTPFPDSSTHRRLFHKSDGSTVSVPMMAQTNKFNYTVFTTPDGHYYDILELPYHGDTLSMFIAAPYEKEVPLSALTNILSAQLISHWKGNMTRLPRLLVLPKFSLETEVDLRKPLENLGMTDMFRQFQADFTSLSDQEPLHVALALQKVKIEVNESGTVASSSTAVIVSARMAPEEIIIDRPFLFVVRHNPTGTVLFMGQVMEP

SEQ ID NO: 196 - protein, 400 aa, Homo sapiens
  misc_feature (1)..(400): Y220S stable mutant human PAI-1
MGSSHHHHHHSSGLVPRGSHMVHHPPSYVAHLASDFGVRVFQQVAQASKDRNVVFSPYGVASVLAMLQLTTGGETQQQIQAAMGFKIDDKGMAPALRHLYKELMGPWNKDEISTTDAIFVQRDLKLVQGFMPHFFRLFRSTVKQVDFSEVERARFIINDWVKTHTKGMISHLLGTGAVDQLTRLVLVNALYFNGQWKTPFPDSSTHRRLFHKSDGSTVSVPMMAQTNKFNYTEFTTPDGHSYDILELPYHGDTLSMFIAAPYEKEVPLSALTNILSAQLISHWKGNMKRLPRLLVLPKFSLETEVDLRKPLENLGMTDMFRQFQADFTSLSDQEPLHVALALQKVKIEVNESGTVASSSTAVIVSARMAPEEIIIDRPFLFVVRHNPTGTVLFMGQVMEP

SEQ ID NO: 197 - protein, 400 aa, Homo sapiens
  misc_feature (1)..(400): Y241S stable mutant human PAI-1
MGSSHHHHHHSSGLVPRGSHMVHHPPSYVAHLASDFGVRVFQQVAQASKDRNVVFSPYGVASVLAMLQLTTGGETQQQIQAAMGFKIDDKGMAPALRHLYKELMGPWNKDEISTTDAIFVQRDLKLVQGFMPHFFRLFRSTVKQVDFSEVERARFIINDWVKTHTKGMISHLLGTGAVDQLTRLVLVNALYFNGQWKTPFPDSSTHRRLFHKSDGSTVSVPMMAQTNKFNYTEFTTPDGHYYDILELPYHGDTLSMFIAAPSEKEVPLSALTNILSAQLISHWKGNMTRLPRLLVLPKFSLETEVDLRKPLENLGMTDMFRQFQADFTSLSDQEPLHVALALQKVKIEVNESGTVASSSTAVIVSARMAPEEIIIDRPFLFVVRHNPTGTVLFMGQVMEP

SEQ ID NO: 198 - protein, 400 aa, Homo sapiens; identical to SEQ ID NO: 191
  misc_feature (1)..(400): R271S stable mutant human PAI-1

SEQ ID NO: 199 - protein, 400 aa, Homo sapiens; identical to SEQ ID NO: 196
  misc_feature (1)..(400): Y220S T267K stable mutant human PAI-1

SEQ ID NO: 200 - protein, 400 aa, Homo sapiens
  misc_feature (1)..(400): cloned HIS-tagged wild type PAI-1
MGSSHHHHHHSSGLVPRGSHMVHHPPSYVAHLASDFGVRVFQQVAQASKDRNVVFSPYGVASVLAMLQLTTGGETQQQIQAAMGFKIDDKGMAPALRHLYKELMGPWNKDEISTTDAIFVQRDLKLVQGFMPHFFRLFRSTVKQVDFSEVERARFIINDWVKTHTKGMISNLLGKGAVDQLTRLVLVNALYFNGQWKTPFPDSSTHRRLFHKSDGSTVSVPMMAQTNKFNYTEFTTPDGHYYDILELPYHGDTLSMFIAAPYEKEVPLSALTNILSAQLISHWKGNMTRLPRLLVLPKFSLETEVDLRKPLENLGMTDMFRQFQADFTSLSDQEPLHVAQALQKVKIEVNESGTVASSSTAVIVSARMAPEEIIMDRPFLFVVRHNPTGTVLFMGQVMEP

SEQ ID NO: 201 - protein, 400 aa, Homo sapiens; identical to SEQ ID NO: 190
  misc_feature (1)..(400): HIS tagged stable PAI-1

SEQ ID NO: 202 - DNA, 30 bp, Artificial sequence
  Synthetic primer HPAI1Nde
ctctcatatggttcaccatcccccatccta

SEQ ID NO: 203 - DNA, 28 bp, Artificial sequence
  Synthetic primer HPAI1XhoI
acacctcgagtcagggttccatcacttg

SEQ ID NO: 204 - DNA, 40 bp, Artificial sequence
  Synthetic primer N150HK154T
ggtatgatcagccacttgcttgggacaggagccgtggacc

SEQ ID NO: 205 - DNA, 28 bp, Artificial sequence
  Synthetic primer Q319L
ctccacgtcgcgctggcgctgcagaaag

SEQ ID NO: 206 - DNA, 30 bp, Artificial sequence
  Synthetic primer M354I
ccgaggagatcatcatcgacagacccttcc

SEQ ID NO: 207 - DNA, 32 bp, Artificial sequence
  Synthetic primer Y220S
acgcccgatggccattcctacgacatcctgga

SEQ ID NO: 208 - DNA, 31 bp, Artificial sequence
  Synthetic primer Y241S
tcattgctgccccttctgaaaaagaggtgcc

SEQ ID NO: 209 - DNA, 31 bp, Artificial sequence
  Synthetic primer E212V
agttcaactatactgtgttcaccacgcccga

SEQ ID NO: 210 - DNA, 31 bp, Artificial sequence
  Synthetic primer R271S
atgaccaggctgcccagcctcctggttctgc

SEQ ID NO: 211 - DNA, 31 bp, Artificial sequence
  Synthetic primer T267K
ggaaaggcaacatgaaaaggctgccccgcct

SEQ ID NO: 212 - DNA, 30 bp, Artificial sequence
  Synthetic primer Y210N
accaacaagttcaacaatactgagttcacc

SEQ ID NO: 213 - protein, 11 aa, Homo sapiens
  misc_feature (4)..(4): Xaa can be any naturally occurring amino acid
  misc_feature (5)..(5): Xaa can be any naturally occurring amino acid
  misc_feature (7)..(7): Xaa can be any naturally occurring amino acid
GIIXXFXGTAN

SEQ ID NO: 214 - protein, 8 aa, Homo sapiens
  misc_feature (2)..(2): Xaa can be any naturally occurring amino acid
  misc_feature (4)..(4): Xaa can be any naturally occurring amino acid
EXRXWLEG

SEQ ID NO: 215 - protein, 6 aa, Homo sapiens
  misc_feature (3)..(3): Xaa can be Asn or Asp
  misc_feature (5)..(5): Xaa can be any naturally occurring amino acid
YSXYXL

SEQ ID NO: 216 - protein, 5 aa, Homo sapiens
  misc_feature (1)..(1): Xaa is a neutral amino acid residue
XYAIS

SEQ ID NO: 217 - protein, 5 aa, Homo sapiens
  misc_feature (1)..(1): Xaa is an amino acid chosen from Ser, Thr, Gly, Ala, Val, Leu, and Ile
XYAIS

SEQ ID NO: 218 - protein, 5 aa, Homo sapiens
  misc_feature (1)..(1): Xaa is an amino acid chosen from Ser and Gly
XYAIS

SEQ ID NO: 219 - protein, 17 aa, Homo sapiens
  misc_feature (5)..(5): Xaa is a neutral amino acid residue
  misc_feature (7)..(7): Xaa is a neutral or acidic amino acid residue
  misc_feature (12)..(12): Xaa is a neutral amino acid residue
  misc_feature (15)..(15): Xaa is a neutral amino residue
  misc_feature (16)..(16): Xaa is a neutral or basic amino acid residue
  misc_feature (17)..(17): Xaa is a neutral amino acid residue
GIIPXFXTANYXQKXXX

SEQ ID NO: 220 - protein, 12 aa, Homo sapiens
  misc_feature (2)..(2): Xaa is a basic amino acid residue
  misc_feature (9)..(9): Xaa is a basic amino acid residue
EXRQWLEGXFDY

SEQ ID NO: 221 - protein, 17 aa, Homo sapiens
  misc_feature (5)..(5): Xaa is an amino acid chosen from Ile, Ser, Thr, Gly, Ala, Val, and Leu
  misc_feature (7)..(7): Xaa is an amino acid chosen from Asp, Asn, Glu, Gln, Gly, Ala, Val, Leu, Ile, Ser, Thr, and Pro
  misc_feature (12)..(12): Xaa is an amino acid chosen from Ala, Ser, Thr, Gly, Val, Leu, and Ile
  misc_feature (15)..(15): Xaa is an amino acid chosen from Phe, Leu, Ile, Ala, Ser, Thr, Gly, Ala, and Val
  misc_feature (16)..(16): Xaa is an amino acid chosen from Gln, Asn, Arg, Lys, and His
  misc_feature (17)..(17): Xaa is an amino acid chosen from Gly, Ser, Thr, Ala, Val, Leu, and Ile
GIIPXFXTANYXQKXXX

SEQ ID NO: 222 - protein, 12 aa, Homo sapiens
  misc_feature (2)..(2): Xaa is an amino acid chosen from Lys, Arg, and His
  misc_feature (9)..(9): Xaa is an amino acid chosen from Lys, Arg, and His
EXRQWLEGXFDY

SEQ ID NO: 223 - protein, 11 aa, Homo sapiens
  misc_feature (1)..(1): Xaa is an amino acid chosen from Arg, Lys, His, Gln, and Asn
  misc_feature (5)..(5): Xaa is an amino acid chosen from Gly, Ser, Thr, Ala, Val, Leu, and Ile
  misc_feature (8)..(8): Xaa is an amino acid chosen from Lys, Arg, and His
  misc_feature (9)..(9): Xaa is an amino acid chosen from Asp, Glu, Asn, and Gln
XASEXIYXXLA

SEQ ID NO: 224 - protein, 7 aa, Homo sapiens
  misc_feature (1)..(1): Xaa is an amino acid chosen from Lys, Arg, and His
  misc_feature (3)..(3): Xaa is an amino acid chosen from Gly, Ser, Thr, Ala, Val, Leu, and Ile
XAXSLAS

SEQ ID NO: 225 - protein, 9 aa, Homo sapiens
  misc_feature (5)..(5): Xaa is an amino acid chosen from Asn, Asp, Glu, and Gln
QQYSXYPLT

SEQ ID NO: 226 - protein, 11 aa, Homo sapiens
  misc_feature (1)..(1): Xaa is a basic or neutral amino acid residue
  misc_feature (5)..(5): Xaa is a neutral amino acid residue
  misc_feature (8)..(8): Xaa is a basic amino acid residue
  misc_feature (9)..(9): Xaa is an acidic or neutral amino acid residue
XASEXIYXXLA

SEQ ID NO: 227 - protein, 7 aa, Homo sapiens
  misc_feature (1)..(1): Xaa is a basic amino acid residue
  misc_feature (3)..(3): Xaa is a neutral amino acid residue
XAXSLAS

SEQ ID NO: 228 - protein, 9 aa, Homo sapiens
  misc_feature (5)..(5): Xaa is an acidic or neutral amino acid residue
QQYSXYPLT

SEQ ID NO: 229 - protein, 17 aa, Homo sapiens
  misc_feature (5)..(5): Xaa is an amino acid chosen from Ile, Thr, and Ala
  misc_feature (7)..(7): Xaa is an amino acid chosen from Asp, Gly, Val, Ser, and Pro
  misc_feature (12)..(12): Xaa is an amino acid chosen from Ala and Ser
  misc_feature (15)..(15): Xaa is an amino acid chosen from Phe and Leu
  misc_feature (16)..(16): Xaa is an amino acid chosen from Gln and Arg
  misc_feature (17)..(17): Xaa is an amino acid chosen from Gly and Ser
GIIPXFXTANYXQKXXX

SEQ ID NO: 230 - protein, 12 aa, Homo sapiens
  misc_feature (2)..(2): Xaa is an amino acid chosen from Lys and Arg
  misc_feature (9)..(9): Xaa is an amino acid chosen from Arg, and His
EXRQWLEGXFDY

SEQ ID NO: 231 - protein, 11 aa, Homo sapiens
  misc_feature (1)..(1): Xaa is an amino acid chosen from Arg and Gln
  misc_feature (5)..(5): Xaa is an amino acid chosen from Gly and Ser
  misc_feature (8)..(8): Xaa is an amino acid chosen from Arg and His
  misc_feature (9)..(9): Xaa is an amino acid chosen from Glu, Asn, Gln, Ser, Thr, Trp, Phe, and Ala
XASEXIYXXLA

SEQ ID NO: 232 - protein, 7 aa, Homo sapiens
  misc_feature (1)..(1): Xaa is an amino acid chosen from Lys and Arg
  misc_feature (3)..(3): Xaa is an amino acid chosen from Ser and Thr
XAXSLAS

SEQ ID NO: 233 - protein, 9 aa, Homo sapiens
  misc_feature (5)..(5): Xaa is an amino acid chosen from Asn and Asp
QQYSXYPLT

SEQ ID NO: 234 - protein, 7 aa, Homo sapiens
  misc_feature (1)..(1): Xaa is a basic amino acid residue
XASSLAS

SEQ ID NO: 235 - protein, 17 aa, Homo sapiens
  misc_feature (5)..(5): Xaa is a neutral amino acid residue
  misc_feature (7)..(7): Xaa is an acidic or neutral amino acid residue
  misc_feature (16)..(16): Xaa is a basic or neutral amino acid residue
GIIPXFXTANYAQKFXG

SEQ ID NO: 236 - protein, 400 aa, Canis familiaris; identical to SEQ ID NO: 123
  misc_feature: Dog PAI-1 amino acid sequence

SEQ ID NO: 237 - protein, 402 aa, Macaca fascicularis; identical to SEQ ID NO: 122
  misc_feature: Cynomolgus PAI-1 amino acid sequence

SEQ ID NO: 238 - protein, 379 aa, Macaca fascicularis
  misc_feature: Cynomolgus PAI-1 amino acid sequence
VHHPPSYVAHLASDFGVRVFQQVAQASKDRNVVFSPYGVASVLAMLQLTTGGETRQQIQAAMGFKIDDKGMAPALRHLYKELLGPWNKDEISTTDAIFVQRDLKLVQGFMPHFFRLFRSTVKQVDFSEAERARFIINDWVKTHTKGMISDLLGKGAVDQLTRLVLVNALYFNGHWKTPFPDSSTHRRLFHKSDGSTVSVPMMAQTNKFNYTEFTTPDGHYYDILELPYHGNTLSMFIAAPYEKQVPLSALTNILSAQLISHWKGNMTRLPRLLVLPKFSLETEVDLRKPLENLGMTDMFRQFQADFTSLSNQEPLHVAQALQKVKIEVNESGTVASSSTAVIVSARMAPEEIIMDRPFLFVVRHNPTGTVLFMGQVMEP

SEQ ID NO: 239 - DNA, 1209 bp, Macaca fascicularis
  misc_feature: Cynomolgus PAI-1 cDNA sequence
atgcagatgtctccagccctcgcctgcctagtcctgggcctggccttcgtctttggtgaagggtccaccgtgcaccatcccccatcctacgtggcccacctggcctcagacttcggggttagggtgtttcagcaggtggcgcaggcctccaaagaccgcaacgtggttttctcaccctatggggtggcctcggtgttggccatgctccagctgacaacgggaggagaaacccggcagcagatccaagcggccatgggattcaagattgatgacaagggcatggctcccgccctccggcatctgtacaaggagctcttggggccgtggaacaaggatgagatcagcaccacagacgcgatctttgtccagcgggatctgaagctggtccagggcttcatgccccacttcttcaggctgttccggagtacggtcaagcaggtggacttttcagaggcagagagagccagattcatcatcaacgactgggtgaagacacacacaaaagggatgatcagtgacttgcttgggaaaggagccgtggaccagctgacacggctggtgctggtgaacgccctctacttcaacggccactggaagactcccttccccgactccagcacccaccgccgcctcttccacaaatcagatggcagcactgtctctgtgcccatgatggctcagaccaacaagttcaactataccgagttcaccacgcccgatggccattactacgacatcctggaactgccctaccacggaaacaccctcagcatgttcattgctgccccttatgaaaagcaggtgcctctctctgccctcaccaacattctgagtgcccagctcatcagccactggaaaggcaacatgaccaggctgccccgcctcctggttctgcccaagttctccctggagactgaagttgacctcaggaagcccctagagaacctgggaatgaccgacatgttcagacaatttcaggcggacttcacgagtctttcaaaccaagagcctctccacgttgcgcaagcgcttcagaaagtgaagatcgaggtgaacgagagtggcacggtggcctcctcatccacagctgtcatagtctcagcccggatggcgcctgaggagatcatcatggacagacccttcctctttgtggtccggcacaaccccacaggaacagtccttttcatgggccaagtgatggaaccttga

SEQ ID NO: 240 - DNA, 747 bp, Homo sapiens
  misc_feature: PICK167_A01 Sequence
ccggccatggcccaggtacagctgcagcagtcaggggctgaagtgaagaagcctggctcttcggtgaaggtctcctgcaaggcttctggaggcaccttcagcagctacgctatcagctgggtgcgacaggcccctggacaaaggcttgagtggatgggagggatcatccctacttttggtacagcaaactacgctcagaagttccagggcagagtcacgattaccgcggacgaatccacgagcacagcctacatggagctgagcggcctgaggtctgaggacacggccgtctattactgtgcgagagagaggaggcagtggctggaaggacactttgactactggggccggggcaccccggtcaccgtctcgagtggtggaggcggctctggcggaggtggcagcggcggtggcggatcggacatccagatgacccagtccccatccttcctgtctgcatctattggagacagagtcaccatcacctgccgggccagtgagggtatttatcacaacttggcctggtatcagcagaagccagggaaagccccaaaactcctgatctataaggcctctagtttagccagtggggccccatcaaggttcagcggcagtagatctgggacagatttcactctcaccatcagcagcctgcagcctgatgattttgcgacttattactgccaacaatatagtaattatccacttactttcggcggagggaccaagctggagatcaaacgtgcg

SEQ ID NO: 241 - DNA, 747 bp, Homo sapiens
  misc_feature: PICK167_A01 Sequence
cgcacgtttgatctccagcttggtccctccgccgaaagtaagtggataattactatattgttggcagtaataagtcgcaaaatcatcaggctgcaggctgctgatggtgagagtgaaatctgtcccagatctactgccgctgaaccttgatggggccccactggctaaactagaggccttatagatcaggagttttggggctttccctggcttctgctgataccaggccaagttgtgataaataccctcactggcccggcaggtgatggtgactctgtctccaatagatgcagacaggaaggatggggactgggtcatctggatgtccgatccgccaccgccgctgccacctccgccagagccgcctccaccactcgagacggtgaccggggtgccccggccccagtagtcaaagtgtccttccagccactgcctcctctctctcgcacagtaatagacggccgtgtcctcagacctcaggccgctcagctccatgtaggctgtgctcgtggattcgtccgcggtaatcgtgactctgccctggaacttctgagcgtagtttgctgtaccaaaagtagggatgatccctcccatccactcaagcctttgtccaggggcctgtcgcacccagctgatagcgtagctgctgaaggtgcctccagaagccttgcaggagaccttcaccgaagagccaggcttcttcacttcagcccctgactgctgcagctgtacctgggccatggccgg

SEQ ID NO: 242 - protein, 248 aa, Homo sapiens
  misc_feature: PICK167_A01 Sequence
PAMAQVQLQQSGAEVKPGSSVKVSCKASGGTFSSYAISWVRQAPGQRLEWMGGIIPTFGTANYAQKFQGRVTITADESTSTAYMELSGLRSEDTAVYYCARERRQWLEGHFDYWGRGTPVTVSSGGGGSGGGGSGGGGSDIQMTQSPSFLSASIGDRVTITCRASEGIYHNLAWYQQKPGKAPKLLIYKASSLASGAPSRFSGSRSGTDFTLTISSLQPDDFATYYCQQYSNYPLTFGGGTKLEIKRA

SEQ ID NO: 243 - DNA, 363 bp, Artificial Sequence; identical to SEQ ID NO: 33
  Synthetic - PICK167_A01-fgl VH

SEQ ID NO: 244 - DNA, 363 bp, Artificial Sequence
  Synthetic - PICK167_A01-fgl VH
tgaggagacggtgaccagggtgccccggccccagtagtcaaagtgtccttccagccactgcctcctctctctcgcacagtaatagacggccgtgtcctcagacctcaggctgctcagctccatgtaggctgtgctcgtggattcgtccgcggtaatcgtgactctgccctggaacttctgagcgtagtttgctgtaccaaaagtagggatgatccctcccatccactcaagcccttgtccaggggcctgtcgcacccagctgatagcgtagctgctgaaggtgcctccagaagccttgcaggagaccttcaccgaagagccaggcttcttcacctcagcccctgactgcaccagctgtacctg

SEQ ID NO: 245 - protein, 121 aa, Artificial Sequence; identical to SEQ ID NO: 34
  Synthetic - PICK167_A01-fgl VH

SEQ ID NO: 246 - DNA, 324 bp, Artificial Sequence
  Synthetic - PICK167_A01-fgl VL
gacatccagatgacccagtccccatccaccctgtctgcatctgttggagacagagtcaccatcacctgccgggccagtgagggtatttatcacaacttggcctggtatcagcagaagccagggaaagccccaaaactcctgatctataaggcctctagtttagccagtggggtcccatcaaggttcagcggcagtagatctgggacagagttcactctcaccatcagcagcctgcagcctgatgattttgcgacttattactgccaacaatatagtaattatccacttactttcggcggagggaccaaggtggagatcaaacgt

SEQ ID NO: 247 - DNA, 324 bp, Artificial Sequence
  Synthetic - PICK167_A01-fgl VL
acgtttgatctccaccttggtccctccgccgaaagtaagtggataattactatattgttggcagtaataagtcgcaaaatcatcaggctgcaggctgctgatggtgagagtgaactctgtcccagatctactgccgctgaaccttgatgggaccccactggctaaactagaggccttatagatcaggagttttggggctttccctggcttctgctgataccaggccaagttgtgataaataccctcactggcccggcaggtgatggtgactctgtctccaacagatgcagacagggtggatggggactgggtcatctggatgtc

SEQ ID NO: 248 - protein, 108 aa, Artificial Sequence
  Synthetic - PICK167_A01-fgl VL
DIQMTQSPSTLSASVGDRVTITCRASEGIYHNLAWYQQKPGKAPKLLIYKASSLASGVPSRFSGSRSGTEFTLTISSLQPDDFATYYCQQYSNYPLTFGGGTKVEIKR

SEQ ID NO: 249 - DNA, 1209 bp, Macaca fascicularis; identical to SEQ ID NO: 239
  misc_feature: Cynomolgus PAI-1 cDNA

SEQ ID NO: 250 - protein, 121 aa, Homo sapiens
QVQLQQSGAEVKKPGSSVKVSCKASGGTFSSYAISWVRQAPGQRLEWMGGIIPTFGTANYAQKFQGRVTITADESTSTAYMELSGLRSEDTAVYYCAREKRQWLEGHFDYWGRGTPVTVSS

SEQ ID NO: 251 - protein, 109 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARWGRGTLVTVSS

SEQ ID NO: 252 - protein, 109 aa, Homo sapiens
DIQMTQSPSFLSASIGDRVTITCRASEGIYHNLAWYQQKPGKAPKLLIYKASSLASGAPSRFSGSRSGTDFTLTISSLQPDDFATYYCQQYSNYPLTFGGGTKLEIKRA

SEQ ID NO: 253 - protein, 104 aa, Homo sapiens
DIQMTQSPSTLSASVGDRVTITCRASQSISSWLAWYQQKPGKAPKLLIYDASSLESGVPSRFSGSGSGTEFTLTISSLQPDDFATYYCQQYNFGGGTKVEIKRA

SEQ ID NO: 254 - protein, 4 aa, Artificial Sequence
  Synthetic peptide
  misc_feature (3)..(3): Xaa is any amino acid
WGXG

SEQ ID NO: 255 - protein, 4 aa, Artificial Sequence
  Synthetic peptide
  misc_feature (3)..(3): Xaa is any amino acid
FGXG

SEQ ID NO: 256 - protein, 5 aa, Artificial Sequence
  Synthetic peptide
GHYYD